(19) United States
(12) Patent Application Publication
(10) Pub. No.: US 2025/0179073 A1
(43) Pub. Date: Jun. 5, 2025

(54) TRICYCLIC HETEROCYCLE COMPOUNDS AS IAP ANTAGONISTS

(71) Applicants: Ascentage Pharma (Suzhou) Co., Ltd., Suzhou (CN); Ascentage Pharma Group Corp Limited, Hong Kong (CN)

(72) Inventors: Jiantao Hu, Suzhou, Jiangsu (CN); Jianyong Chen, Suzhou, Jiangsu (CN); Fang Liu, Suzhou, Jiangsu (CN); Leilei Zhao, Suzhou, Jiangsu (CN); Baolin He, Suzhou, Jiangsu (CN); Hao Chen, Suzhou, Jiangsu (CN); Xianchan Zha, Suzhou, Jiangsu (CN); Rubin Zhou, Suzhou, Jiangsu (CN); Jun Zhang, Suzhou, Jiangsu (CN); Jiawei Wang, Suzhou, Jiangsu (CN); Huifang Shi, Suzhou, Jiangsu (CN)

(21) Appl. No.: 18/842,145

(22) PCT Filed: Mar. 10, 2023
(86) PCT No.: PCT/CN2023/080856

§ 371 Date: Aug. 28, 2024

(30) Foreign Application Priority Data
Mar. 11, 2022 (WO) PCT/CN2022/080359
Mar. 3, 2023 (CN) 202310193921.3

(51) Int. Cl. C07D 471/14; A61K 31/496; A61K 31/5377; A61K 31/538; A61K 31/5386; A61K 31/541; A61K 31/675; C07D 487/04; C07D 491/22; C07D 513/04; C07D 519/00; C07F 9/6561

(52) U.S. Cl. CPC C07D 471/14; A61K 31/496; A61K 31/5377; A61K 31/538; A61K 31/5386; A61K 31/541; A61K 31/675; C07D 487/04; C07D 491/22; C07D 513/04; C07D 519/00; C07F 9/6561

(57) ABSTRACT

The present disclosure relates to compounds that inhibit IAP (preferably cIAP1, cIAP2 or XIAP) protein, pharmaceutical compositions comprising the same and methods of using the IAP protein inhibitors in the treatment of diseases and conditions wherein inhibition of IAP protein provides a benefit.

### TECHNICAL FIELD

The present disclosure relates to new inhibitors/antagonists of Inhibitors of Apoptosis Proteins (IAP) and to therapeutic methods of treating conditions and diseases wherein inhibition/antagonism of IAP proteins provides a benefit. The present inhibitors effectively bind to IAP proteins, including cIAP1, cIAP2, and XIAP.

### BACKGROUND OF THE INVENTION

Apoptosis, or programmed cell death, is a cell process critical for homeostasis, normal development, host defense, and suppression of oncogenesis. Faulty regulation of apoptosis has been implicated in many human diseases, including cancer, and it is now recognized that resistance to apoptosis is a hallmark of cancer. As a consequence, targeting of key apoptosis regulators has emerged as an attractive strategy for the development of new approaches to human cancer treatment.

Most current cancer therapies, including chemotherapeutic agents, radiation, and immunotherapy, indirectly induce apoptosis in cancer cells. The inability of cancer cells to execute an apoptotic program due to defects in the normal apoptotic machinery is thus often associated with an increase in resistance to chemotherapy, radiation, or immunotherapy-induced apoptosis. Such primary or acquired resistance of human cancers to current therapies due to apoptosis defects is a major problem in current cancer therapy.

In order to improve survival and quality of life of cancer patients, current and future efforts in the design and development of new molecular target-specific anticancer therapies includes strategies that specifically target cancer cell resistance to apoptosis. In this regard, targeting negative regulators that play a central role in directly inhibiting apoptosis in cancer cells represents a highly promising therapeutic strategy for new anticancer drug design.

One class of central negative regulators of apoptosis is the Inhibitors of Apoptosis Proteins (IAPs). The family of IAP proteins comprises 8 members, XIAP, cIAP1, cIAP2, NAIP, ILP2, ML-IAP, survivin and BRUCE (also known as apollon). Members of the IAP family have been shown to inhibit programmed cell death through their ability to directly inhibit members of the caspase family of apoptotic enzymes, although the precise role of all 8 members is yet to be fully defined. The common structural feature of all IAP family members is a −70 amino acid zinc-binding fold termed the baculovirus IAP repeat (BIR) domain, which is present in one to three copies.

Many interactions between IAPs and other proteins are mediated via a surface groove on the BIR domain. BIR domains may be classified by their peptide-binding specificity. There are three types of BIR domains; type III domains (capable of binding caspase (and caspase-like) peptides with a specificity for proline in the third (P3) position (e.g. XIAP BIR3), type II domains (like type III domains but lacking the proline requirement e.g. XIAP BIR2) and type I domains (which do not bind caspases or similar peptides, e.g. XIAP BIR1) (Eckelman et al. Cell Death and Differentiation 2008; 15: 920-928). BIRs are small (−70 amino acids) Zn-coordinated domains and a variety of proteins use their N-terminal to interact with the BIR domains grooves. BIR antagonists prevent caspases binding to BIRs and hence result in increased caspase activity thereby inducing auto-ubiquitination and proteasomal degradation of IAPs.

IAPs are overexpressed in many cancers including renal, melanoma, colon, lung, breast, ovarian and prostate cancers (Tamm et al., Clin. Cancer Research 2000; 6(5): 1796-803), and have been implicated in tumour growth, pathogenesis and resistance to chemo- and radio-therapy (Tamm 2000).

Small molecule inhibitors of IAP proteins are known. For example, U.S. Patent Publication Application No. 2005/0197403 and U.S. Pat. No. 7,960,372 disclose dimeric Smac mimetic compounds, each incorporated herein by reference in its entirety.

Despite the discovery of small molecule inhibitors of TAP proteins, the design of potent inhibitors of TAP proteins remains a significant challenge in modern drug discovery. Accordingly, a need still exists in the art for IAP inhibitors having physical and pharmacological properties that permit use of the inhibitors in therapeutic applications. The present invention provides compounds designed to bind to TAP proteins and inhibit IAP protein activity.

### SUMMARY OF THE INVENTION

In one aspect, the present disclosure provides compounds represented by Formula I, II, II-A, II-B or II-C below, and the pharmaceutically acceptable salts and solvates, e.g., hydrates, thereof, collectively referred to as “compounds of the disclosure.” Compounds of the disclosure are IAP (especially cIAP1, cIAP2, or XIAP) inhibitors and are thus useful in treating or preventing diseases or conditions such as TAP-associated cancer wherein the inhibition of TAP provides a benefit.

In another aspect, the present disclosure provides pharmaceutical compositions comprising the compound of the disclosure, and a pharmaceutically acceptable carrier.

In another aspect, the present disclosure provides methods for inhibiting TAP protein activity in a cell, comprising contacting the cell in which inhibition of TAP protein activity is desired with an effective amount of a compound of the disclosure, or a pharmaceutically acceptable salt or solvate thereof; or a pharmaceutical composition of the disclosure.

In another aspect, the present disclosure provides methods of treating a disease or condition wherein inhibition of an IAP protein provides a benefit comprising administering a therapeutically effective amount of a compound of the disclosure, or a pharmaceutically acceptable salt or solvate thereof; or a pharmaceutical composition of the disclosure.

In one embodiment, the disease or condition is a cancer. In another embodiment, the disease or condition is selected from the group consisting of T and B cell mediated autoimmune diseases; inflammatory diseases; infections; hyperproliferative diseases; AIDS; degenerative conditions; vascular diseases; and the like. In another embodiment, the disease or condition is selected from the group consisting of HBV, autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjogren's syndrome, systemic lupus erythematosus, type 1 diabetes, ulcerative colitis, vitiligo, and the like.

In another aspect, the present disclosure provides compounds or pharmaceutical compositions of the disclosure for use in inhibiting IAP protein activity in a cell.

In another aspect, the present disclosure provides compounds or pharmaceutical compositions of the disclosure for use in treating a disease or condition wherein inhibition of an IAP protein provides a benefit.

In another aspect, the present disclosure provides the use of compounds or pharmaceutical compositions of the disclosure in the manufacture of a medicament for inhibiting IAP protein activity in a cell.

In another aspect, the present disclosure provides the use of compounds or pharmaceutical compositions of the disclosure in the manufacture of a medicament for treating a disease or condition wherein inhibition of an IAP protein provides a benefit.

In another aspect, the present disclosure provides kits comprising compounds of the disclosure, and instructions for administering the compounds of the disclosure to a subject (e.g., a patient) for which the inhibition of an IAP protein provides a benefit.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE INVENTION

The present disclosure relates to compounds that inhibit IAP. In particular, the present disclosure relates to compounds that inhibit the activity of cIAP1, cIAP2, or XIAP, pharmaceutical compositions comprising the same and methods of use therefor.

Unless otherwise defined below, all technical and scientific terms used herein have the same meanings as commonly understood by one of ordinary skill in the art. References to techniques used herein are intended to refer to techniques that are generally understood in the art, including those obvious changes or equivalent replacements of the techniques for those skilled in the art. While it is believed that the following terms are well understood by those skilled in the art, the following definitions are set forth to better explain the disclosure.

### I. Definitions

As used herein, the terms “including”, “comprising”, “having”, “containing” or “comprising”, and other variants thereof, are inclusive or open, and do not exclude other unlisted elements or method steps.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “IAP proteins,” as used herein, refers to any known member of the Inhibitors of Apoptosis Protein family, including, but not limited to, XIAP, cIAP-1, cIAP-2, ML-IAP, HIAP, TSIAP, KIAP, NAIP, survivin, livin, ILP-2, apollon, and BRUCE.

The term “overexpression of IAP” as used herein, refers to an elevated level (e.g., aberrant level) of mRNAs encoding for an IAP protein(s), and/or to elevated levels of IAP protein(s) in cells as compared to similar corresponding non-pathological cells expressing basal levels of mRNAs encoding IAP proteins or having basal levels of IAP proteins.

The term “a disease or condition wherein inhibition of an IAP protein provides a benefit” pertains to a condition in which an IAP protein, and/or an action of an IAP protein, is important or necessary, e.g., for the onset, progress, expression of that disease or condition, or a disease or a condition which is known to be treated by an IAP protein inhibitor. An example of such a condition includes, but is not limited to, a cancer. One of ordinary skill in the art is readily able to determine whether a compound treats a disease or condition mediated by an IAP protein for any particular cell type, for example, by assays which conveniently can be used to assess the activity of particular compounds.

The term “disease” or “condition” denotes disturbances and/or anomalies that as a rule are regarded as being pathological conditions or functions, and that can manifest themselves in the form of particular signs, symptoms, and/or malfunctions. As demonstrated below, a compound of structural formula (I) is a potent inhibitor of IAP proteins and can be used in treating diseases and conditions wherein inhibition an IAP protein provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound of the invention to an individual in need of such treatment.

Within the meaning of the invention, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “inhibitor(s)” and “antagonist(s)” are used interchangeably herein, and refer to any agent that has inhibitory or antagonistic activity on IAP protein, including but not limited to small molecule compounds, biological macromolecules, and the like. For the same reason, the term “inhibition” and “antagonism” are also used interchangeably herein.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented.

The term “therapeutically effective amount” or “effective dose” as used herein refers to an amount of the active ingredient(s) that is (are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “halo” or “halogen” as used herein by itself or as part of another group refers to —Cl, —F, —Br, or —I.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl. In another embodiment, one or more of the hydrogen atoms of the alkyl group are replaced by deuterium atoms, i.e., the alkyl group is isotopically-labeled with deuterium. A non-limiting exemplary deuterated alkyl group is —CD₃. In another embodiment, none of the hydrogen atoms of the alkyl group are replaced by deuterium atoms, i.e., the alkyl group is isotopically-labeled with deuterium.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxy)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group and resulting alkoxy is thus referred to as a C₁-C₄ alkoxy. Non-limiting exemplary alkoxy groups include methoxy, ethoxy, and tert-butoxy.

The term “carbocyclic” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ carbocyclic. For example, a C₅ carbocyclic or a C₆ carbocyclic. When the aliphatic hydrocarbons are saturated, carbocyclic may also be called as cycloalkyl, e.g., a C₃ cycloalkyl such a cyclopropyl, a C₄ cycloalkyl such as cyclobutyl, etc. In one embodiment, the carbocyclic is cycloalkyl. In one embodiment, the cycloalkyl is bicyclic, i.e., it has two rings. In another embodiment, the cycloalkyl is monocyclic, i.e., it has one ring. In another embodiment, the cycloalkyl is a C₃_s cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “heterocyclyl” as used herein by itself or as part of another group refers to saturated and partially unsaturated, e.g., containing one or two double bonds, monocyclic, bicyclic, or tricyclic groups containing three to fourteen ring members, i.e., a 4- to 14-membered heterocyclyl, comprising one, two, three, or four heteroatoms, for example 4-membered, 5-membered, 6-membered, 7-membered, 8-membered, 9-membered, 10-membered heterocyclo. Each heteroatom is independently oxygen, sulfur, or nitrogen. Each sulfur atom is independently oxidized to give a sulfoxide, i.e., S(═O), or sulfone, i.e., S(═O)₂.

The term heterocyclyl includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclyl also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclyl group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclyl groups include:

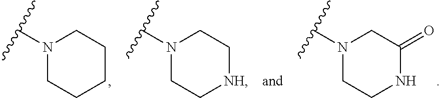

The term “aryl” as used herein by itself or as part of another group refers to an aromatic ring system having six to fourteen carbon atoms, i.e., C₆-C₁₄ aryl, C₉-C₁₀ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “heteroaryl” as used herein by itself or as part of another group refers to monocyclic and bicyclic aromatic ring systems having five to fourteen ring members, i.e., a 5- to 14-membered heteroaryl, a 5- to 10-membered-heteroaryl, a 5- to 7-membered (preferably 5-membered or 6-membered) comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups. Non-limiting exemplary (cycloalkyl)alkyl groups include:

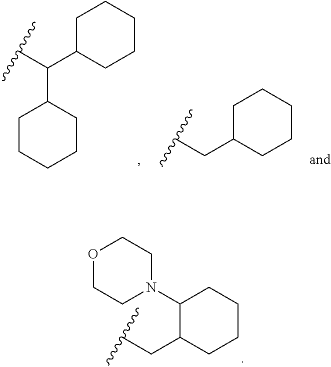 [SMILES: *C(C1CCCCC1)C1CCCCC1 ; *CC1CCCCC1 ; *CC1CCCCC1N1CCOCC1]

The term “carboxy” as used by itself or as part of another group refers to a radical of the formula —C(═O)OH.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom. Non-limiting exemplary (heterocyclo)alkyl groups include:

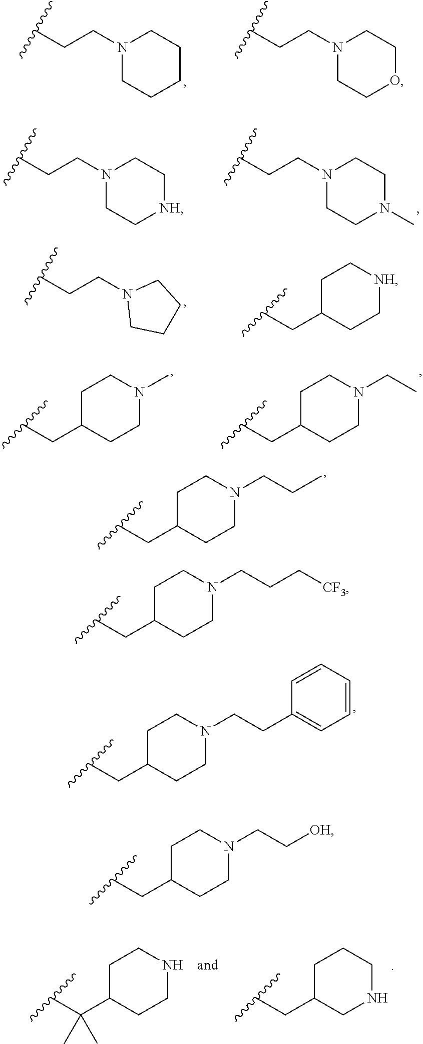

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl. Non-limiting exemplary (heteroaryl)alkyl groups include:

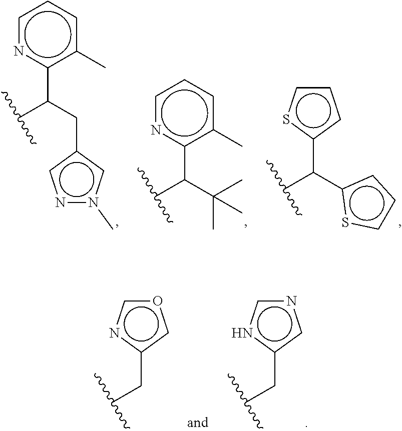

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a compound wherein substantially all of the atoms at a position within the Compound of the Disclosure are replaced by an atom having a different atomic mass or mass number. In another embodiment, provided is a compound wherein substantially all of the atoms at a position within the Compound of the Disclosure are replaced by deuterium atoms, e.g., all of the hydrogen atoms of a —CH₃ group are replaced by deuterium atoms to give a —CD₃ group. In another embodiment, provided is a compound wherein a portion of the atoms at a position within the Compound of the disclosure are replaced, i.e., the Compound of the Disclosure is enriched at a position with an atom having a different atomic mass or mass number. In another embodiment, provided is a compound wherein none of the atoms of the Compound of the Disclosure are replaced by an atom having a different atomic mass or mass number. Isotopically-labelled Compounds of the Disclosure can be prepared by methods known in the art.

Compounds of the Disclosure contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number ±10%. Thus, “about 10” means 9 to 11.

The phrase “optionally substituted with one or more groups” as used herein refers to optionally substituted with 1-10 groups, preferably optionally substituted with 1-5 groups, and more preferably optionally substituted with 1, 2, or 3 groups.

### II. Compounds

In one aspect, the present disclosure provides a compound of Formula I:

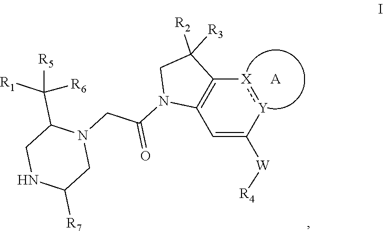

wherein:
W is O, S, S(O)₂, NR^{a}, —C(═O) or CR^{a}R^{b}, wherein R^{a} and R^{b} are each independently selected from H, halo, C₁-C₆ alkyl, OH or C₃-C₆ cycloalkyl;
X and Y are each independently C or N;
----- represents a single bond or a double bond;
i.e, when ----- represents a single bond, this means that X and Y are linked by a single bond (X—Y); when ----- represents a double bond, this means that X and Y are linked by a double bond (X═Y).
R₁ is selected from heterocyclyl or heteroaryl optionally substituted with one or more groups selected from R_{1A}; and R_{1A} is selected from CN, —C₁-C₆ alkyl-CN, —C(═O)—NH₂, halo, OH, COOH, NH₂, oxo, C₁-C₆ alkyl, C₁-C₆ haloalkyl, C₁-C₆ alkoxyl, —C(═O)—C₁-C₆ alkyl, —C(═O)—O—C₁-C₆ alkyl, —C₁-C₆ alkyl-C₁-C₆ alkoxy, —C₁-C₆ alkyl-OH, —C₁-C₆ alkyl-NH₂, —S(O)₂—C₁-C₆ alkyl, or 4-6-membered heterocyclyl; or two R_{1A} together with the carbon atom to which they are attached form a C₃-C₆ cycloalkyl or —C(═O);
R₂ and R₃ are each independently selected from H, or C₁-C₆ alkyl; or R₂ and R₃ together with the carbon atom to which they are attached form a C₃-C₆ cycloalkyl or C₃-C₆ heterocyclyl;
R₄ is selected from phenyl, C₂-C₆ alkyl, 3-6-membered cycloalkyl, 3-6-membered heterocycloalkyl or 5-6-membered heteroaryl optionally substituted with one or more R₄ₐ, or C₁-C₆ alkyl or —C₁-C₆ alkyl-C₃-C₆ cycloalkyl; and R₄ₐ is selected from CN, halo, OH, NH₂, oxo, COOH, C₁-C₆ alkyl, C₁-C₆ haloalkyl, C₁-C₆ alkoxyl; optionally, R^{a} and R^{4a} together with the atoms to which they are attached may form a 5-6-membered carbocyclyl, 5-6-membered heterocyclyl or 5-6-membered heteroaryl optionally substituted with one or more of halo, C₁-C₆ alkyl, or OH;
R₅ and R₆ are each independently selected from H, or C₁-C₆ alkyl;
R₇ is selected from C₁-C₆ alkyl, preferably C₁-C₃ alkyl;
A ring is selected from 5-8-membered aryl, 5-8-membered heteroaryl or 5-8-membered heterocyclyl optionally substituted with one or more groups selected from R₈; and R₈ is selected from C₁-C₆ alkyl, deuterated C₁-C₆ alkyl, C₁-C₆ haloalkyl, —C₁-C₆ haloalkyl-OH, C₁-C₆ alkoxy, C₁-C₆ alkyl(C₁-C₆ alkoxy)-C₁-C₆ alkoxy, C₃-C₆ cycloalkyl, —C₁-C₆ alkyl-C₃-C₆ cycloalkyl, oxo, 4-7-membered heterocyclyl, —NH-4-7-membered heterocyclyl, —C₁-C₆ alkyl-4-7-membered heterocyclyl, —C₁-C₆ alkyl(OH)-4-7-membered heterocyclyl, CN, —C₁-C₆ alkyl-CN, —COGH, —OH, amino, halo, —NH—S(O)₂—C₁-C₆ alkyl, —N(S(O)₂—C₁-C₆ alkyl)₂, —C₁-C₆ alkyl-OH, —C₁-C₆ haloalkyl-OH, —C₁-C₆ alkyl (OH)—OH, —S(O)₂—C₁-C₆ alkyl, —C₁-C₆ alkyl-S(O)₂—C₁-C₆ alkyl, —P(O)—(C₁-C₆ alkyl)₂, —C₁-C₆ alkyl-N(C₁-C₆ alkyl)₂, —C(═O)—NH₂, —C₁—C alkyl-C(═O)—NH₂, —C(═O)—NH—C₁-C₆ alkyl, —C(═O)—NH—C₃-C₆ cycloalkyl, —C(═O)—NH-(deuterated C₁-C₆ alkyl), —C(═O)—N(C₁-C₆ alkyl)₂, —C(═O)—O—C₁-C₆ alkyl, —C(═O)—C₁-C₆ alkyl, —NH—C(═O)—C₁-C₆ alkyl, —NH—C(═O)—H, —N(C₁-C₆ alkyl)-C(═O)—H, —NH—C(═O)—O—C₁-C₆ alkyl, —N(C₁-C₆ alkyl)-C(═O)—C₁-C₆ alkyl, 5-6-membered heteroaryl, —C₁-C₆ alkyl-(5-6-membered heteroaryl), or phenyl, wherein the C₃-C₆ cycloalkyl, 4-7-membered heterocyclyl, 5-6-membered heteroaryl or phenyl is optionally substituted with one or more R₈ₐ, and R₈ₐ is selected from halo, —OH, C₁-C₆ alkyl, or oxo;
or A ring is absent;
or a pharmaceutically acceptable salt or solvate thereof.

In another aspect, the present disclosure provides a compound of Formula I:

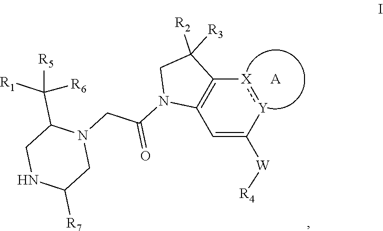

wherein:
W is O, S, NR^{a}, or CR^{a}R^{b}, wherein R^{a} and R^{b} are each independently selected from H, halo, C₁-C₆ alkyl, OH or C₃-C₆ cycloalkyl;
X and Y are each independently C or N;
[symbol] represents a single bond or a double bond;
i.e, when [symbol] represents a single bond, this means that X and Y are linked by a single bond (X—Y); when [symbol] represents a double bond, this means that X and Y are linked by a double bond (X═Y).
R₁ is selected from heterocyclyl or heteroaryl optionally substituted with one or more (e.g., 1-10, 1-5, 1-3 or 1-2) groups selected from R_{1A}; and R_{1A} is selected from CN, halo, OH, COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH, —C₁-C₆ alkyl-NH₂, or two R_{1A} together with the carbon atom to which they are attached form a C₃-C₆ cycloalkyl or —C(═O);
R₂ and R₃ are each independently selected from H, or C₁-C₆ alkyl; or R₂ and R₃ together with the carbon atom to which they are attached form a C₃-C₆ cycloalkyl;
R₄ is selected from phenyl or pyridyl optionally substituted with one or more (e.g., 1-10, 1-5, 1-3 or 1-2) R₄ₐ, or C₁-C₆ alkyl or —C₁-C₆ alkyl-C₃-C₆ cycloalkyl; and
R₄ₐ is selected from CN, halo, OH, NH₂, COOH, C₁-C₆ alkyl, C₁-C₆ haloalkyl, C₁-C₆ alkoxyl;
R₅ and R₆ are each independently selected from H, or C₁-C₆ alkyl;
R₇ is selected from C₁-C₆ alkyl, preferably C₁-C₃ alkyl;
A ring is selected from 5-8 membered (preferably 5 or 6 membered) aryl or 5-8-membered (preferably 5 or 6 membered) heteroaryl optionally substituted with one or more groups selected from R₈; and R₈ is selected from C₁-C₆ alkyl, C₁-C₆ haloalkyl, C₁-C₆ alkoxy, C₃-C₆ cycloalkyl, 5- or 6-membered heterocyclyl containing one or more heteroatoms selected from N, O and S (e.g., tetrahydropyranyl), CN, COOH, OH, amino, halo, —NH—S(O)₂—C₁-C₆ alkyl, —N(S(O)₂—C₁-C₆ alkyl)₂, —C₁-C₆ alkyl-OH, —C(═O)—NH₂, —C(═O)—NH—C₁-C₆ alkyl, —C(═O)—O—C₁-C₆ alkyl, —NH—C(═O)—C₁-C₆ alkyl, phenyl optionally substituted with one or more halogens;
or a pharmaceutically acceptable salt or solvate thereof.

In another aspect, the present disclosure provides a compound of Formula II:

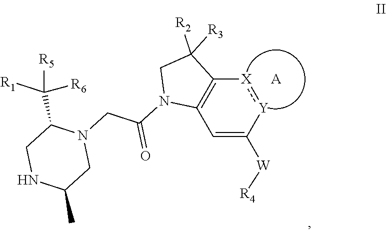

wherein all the groups are as defined above,
or a pharmaceutically acceptable salt or solvate thereof.

In another aspect, the present disclosure provides a compound is of Formula II-A, II-B or II-C;

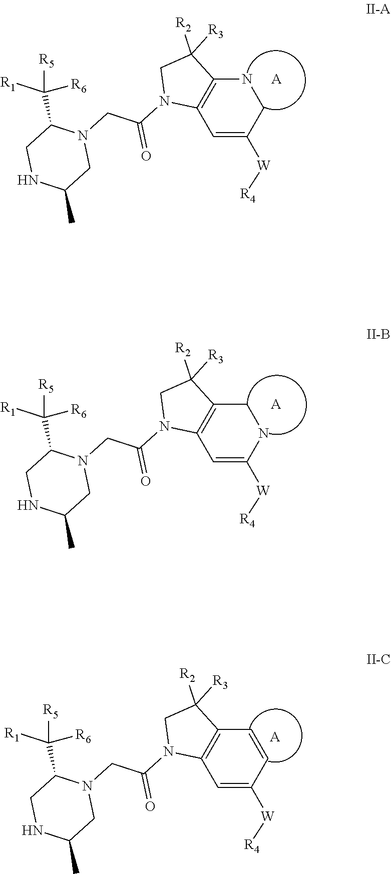

wherein all the groups are as defined above,
or a pharmaceutically acceptable salt or solvate thereof.

In some embodiments, the A ring contains 1, 2 or 3 nitrogen atoms.

In some embodiments, X is N, Y is C, and the

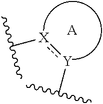

is selected from:

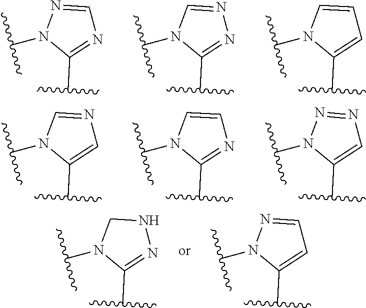

optionally substituted with one or more groups selected from R₈.

In some embodiments, X is N, Y is C, and the

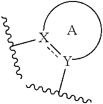

is selected from:

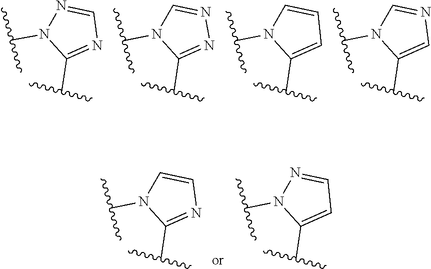

optionally substituted with one or more groups selected from R₈.

In some embodiments, X is C, Y is N, and the

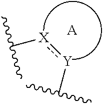

is selected from:

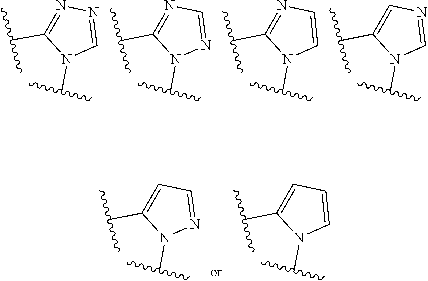

optionally substituted with one or more groups selected from R₈.

In some embodiments, X is C, Y is C, and the

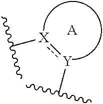

is selected from:

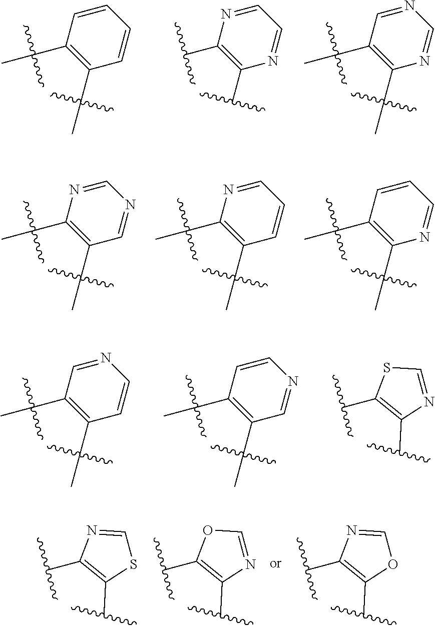

optionally substituted with one or more groups selected from R₈.

In some embodiments,

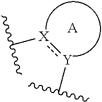

is selected from the group consisting of:

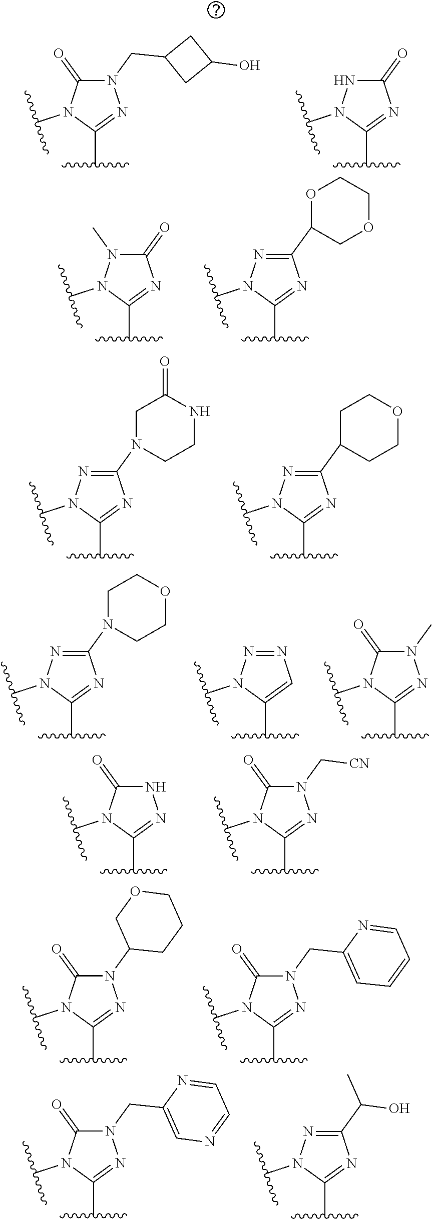

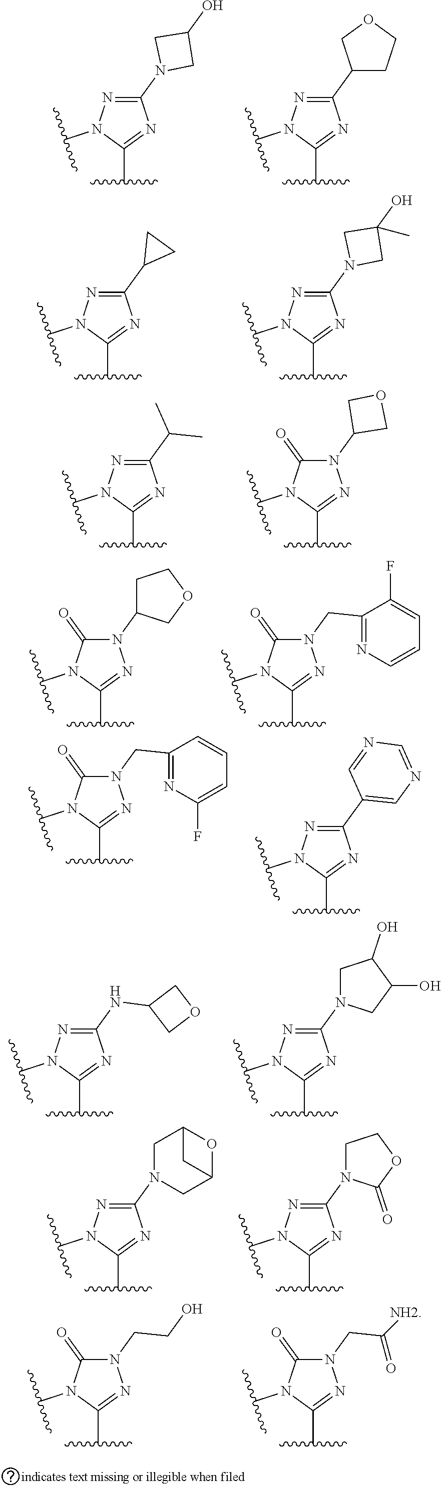

In some embodiments,

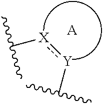

is selected from the group consisting of:

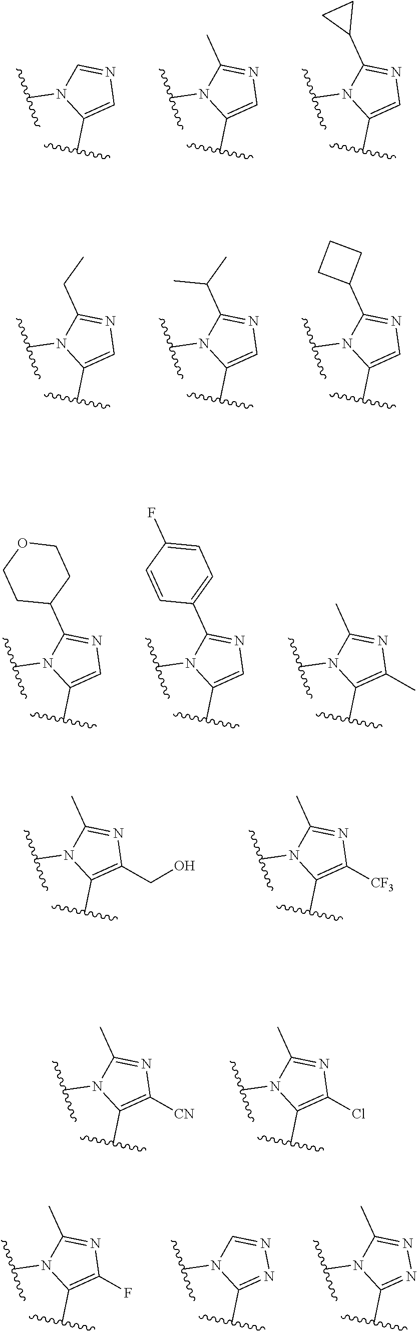

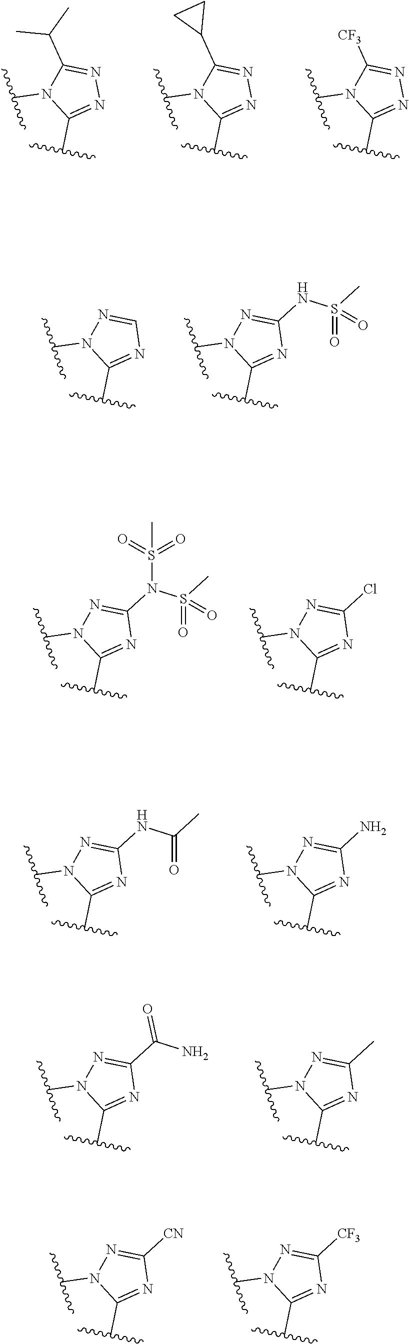

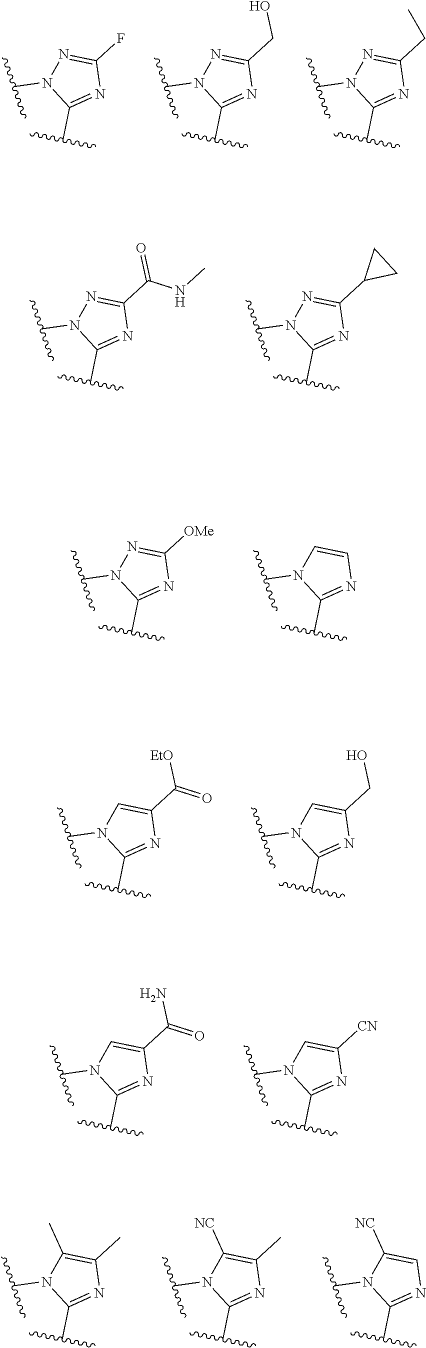

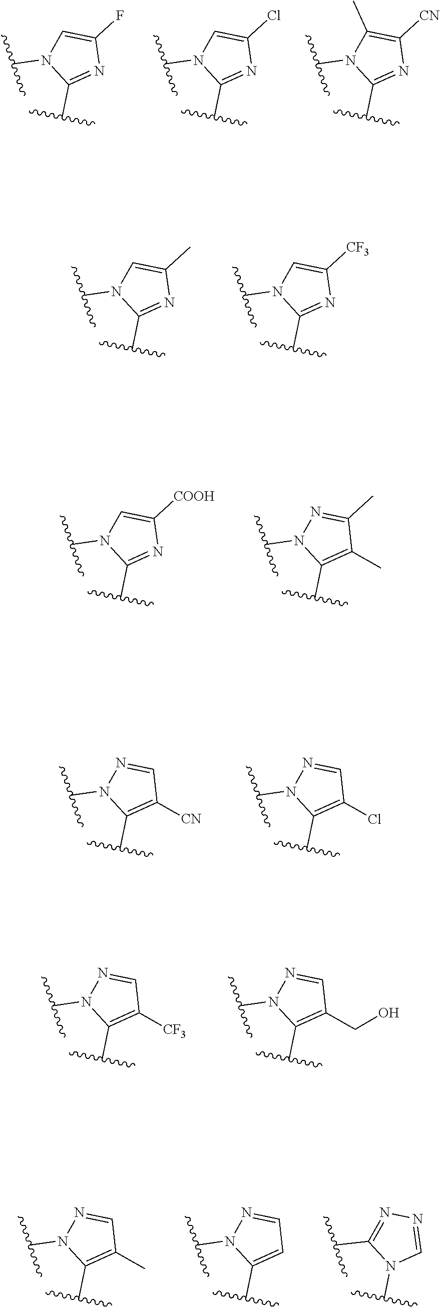

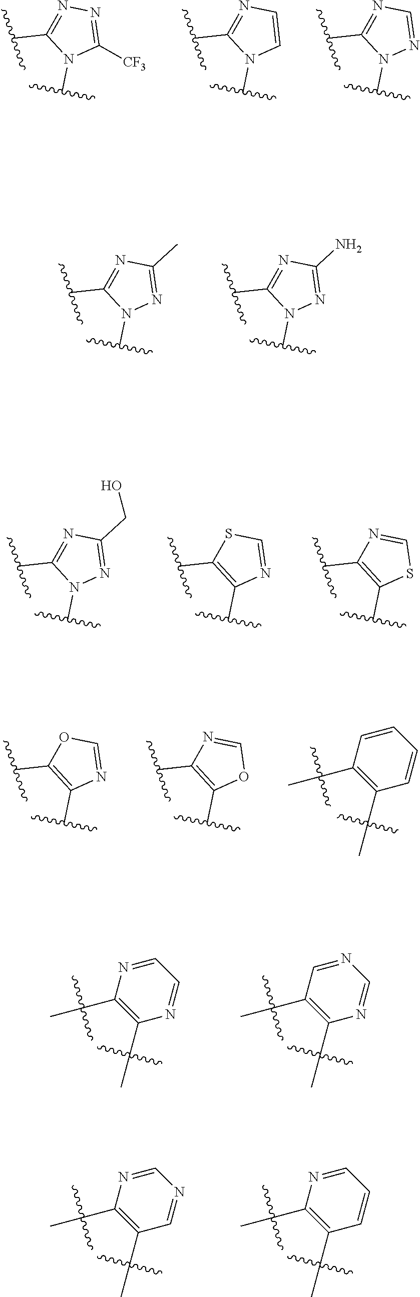

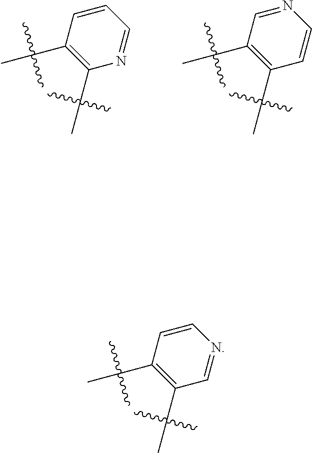 [SMILES: *c1cccnc1* ; *c1ccncc1* ; *c1ccncc1*]

In some embodiments, R₁ is of Formula III:

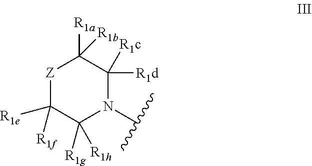

wherein:
Z is selected from O, S, NR^{a}, or CR^{a}R^{b}, wherein R^{a} and R^{b} are each independently selected from CN, halo, OH, COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH or —C₁-C₆ alkyl-NH₂;
R₁ₐ, R_{1b}, R_{1c}, R_{1d}, R₁ₑ, R_{1f}, R_{1g}, and R₁ₕ are independently selected from CN, halo, OH, COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH, —C₁-C₆ alkyl-CN, —S(O)₂—C₁-C₆ alkyl, 4-7-membered heterocyclyl, oxo, —C(═O)—NH₂, —C(═O)—O—C₁-C₆ alkyl, or —C₁-C₆ alkyl-NH₂; or
R₁ₐ and R_{1b}, R_{1c} and R_{1d}, R_{1c} and R_{1f} and/or R_{1g} and R_{1b}, together with the carbon atom to which they are attached form a C₃-C₆ cycloalkyl; or
any two of R₁ₐ, R_{1b}, R_{1c}, R_{1d}, R_{1c}, R_{1f}, R_{1g}, and R₁ₕ on different carbon atoms are linked together through a —(CH₂)ₙ— linker to form a bridged ring, wherein n is 1, 2 or 3.

In some embodiments, R₁ is of Formula III:

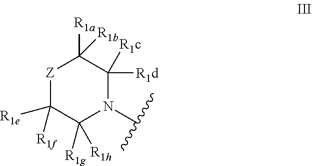

wherein:
Z is selected from O, S, NR^{a}, or CR^{a}R^{b}, wherein R^{a} and R^{b} are each independently selected from CN, halo, OH, COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH or —C₁-C₆ alkyl-NH₂;
R₁ₐ, R_{1b}, R_{1c}, R_{1d}, R₁ₑ, R_{1f}, R_{1g}, and R₁ₕ are independently selected from CN, halo, OH, COOH, NH₂, C₁-C₆ alkyl, C₁-C₆ haloalkyl, C₁-C₆ alkoxyl, —C(O)—C₁-C₆ alkyl, —C₁-C₆ alkyl-OC₁-C₆ alkyl, —C₁-C₆ alkyl-OH or —C₁-C₆ alkyl-NH₂; or
R₁ₐ and R_{1b}, R_{1c} and R_{1d}, R₁ₑ and R_{1f} and/or R_{1g} and R₁ₕ, together with the carbon atom to which they are attached form a C₃-C₆ cycloalkyl; or
any two of R₁ₐ, R_{1b}, R_{1c}, R_{1d}, R_{1c}, R_{1f}, R_{1g}, and R₁ₕ on different carbon atoms are linked together through a —(CH₂)ₙ— linker to form a bridged ring, wherein n is 1, 2 or 3.

In some embodiments, R₁ is selected from the group consisting of:

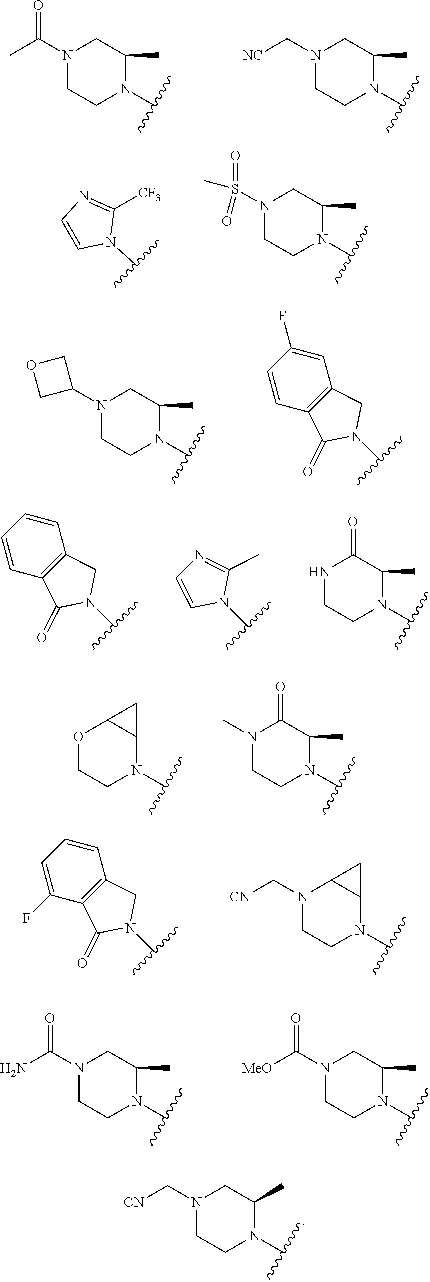

In some embodiments, R₁ is selected from the group consisting of:

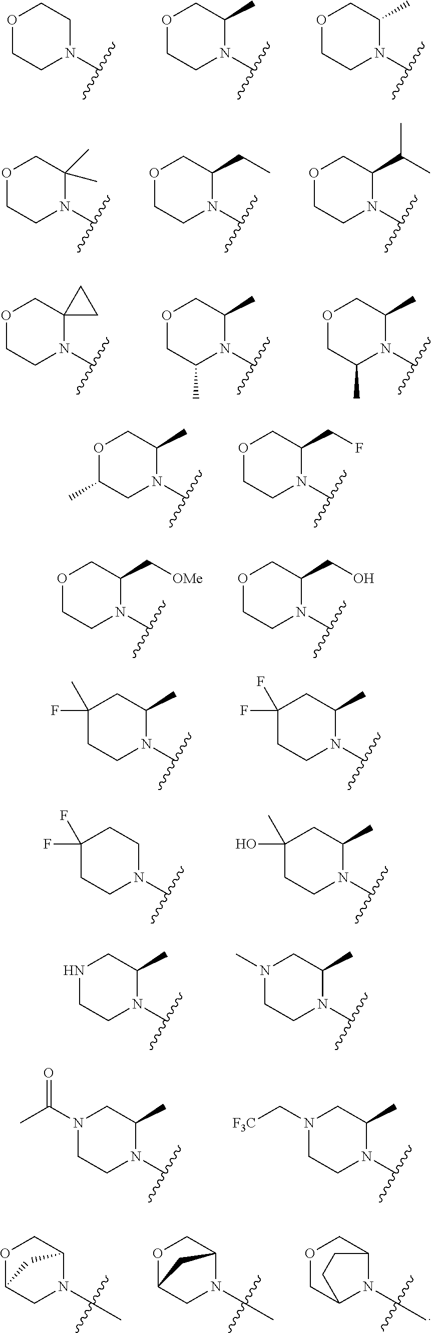

In some embodiments, R₁ is selected from the group consisting of:

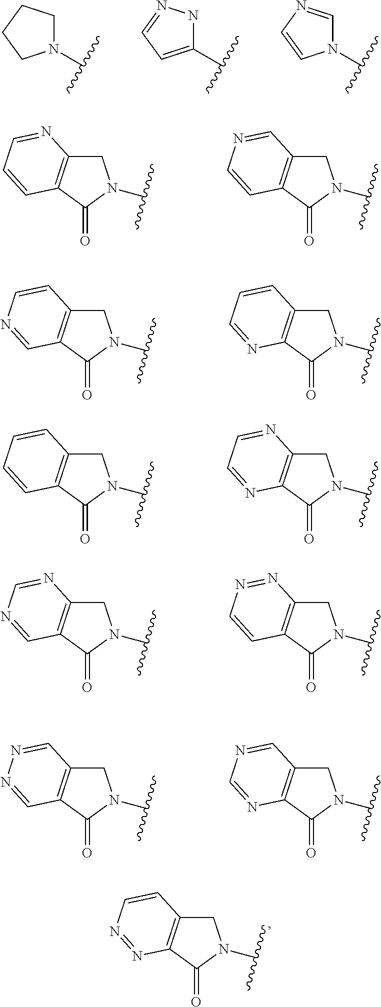

optionally substituted with one or more groups selected from CN, halo, OH, —COGH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH, and —C₁-C₆ alkyl-NH₂.

In some embodiments, R₁ is selected from the group consisting of:

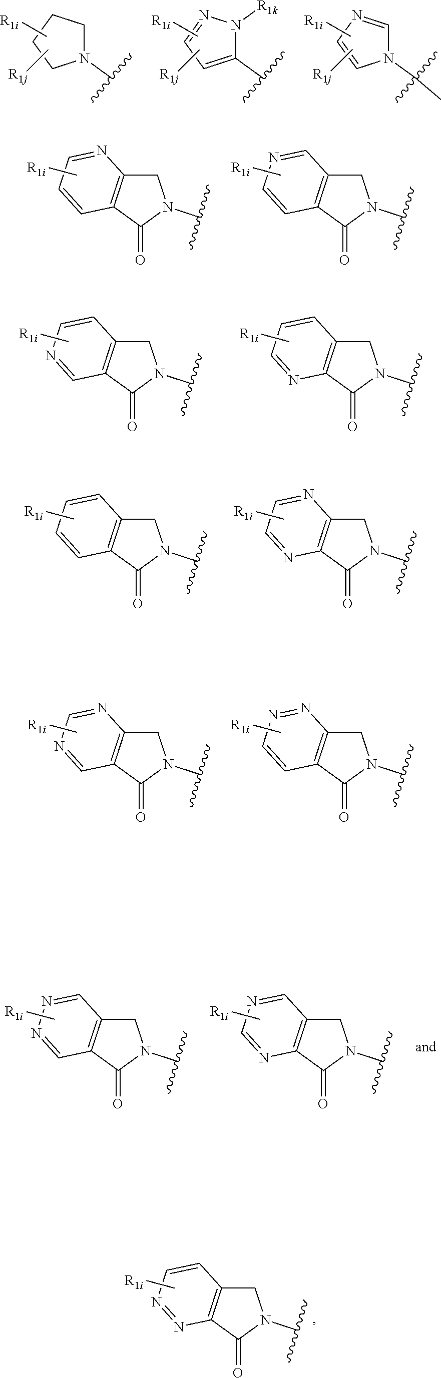

wherein R₁ᵢ; R₁ⱼ and R_{1K} are independently selected from CN, halo, OH, —COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH, and —C₁-C₆ alkyl-NH₂.

In some embodiments, R₁ is selected from the group consisting of:

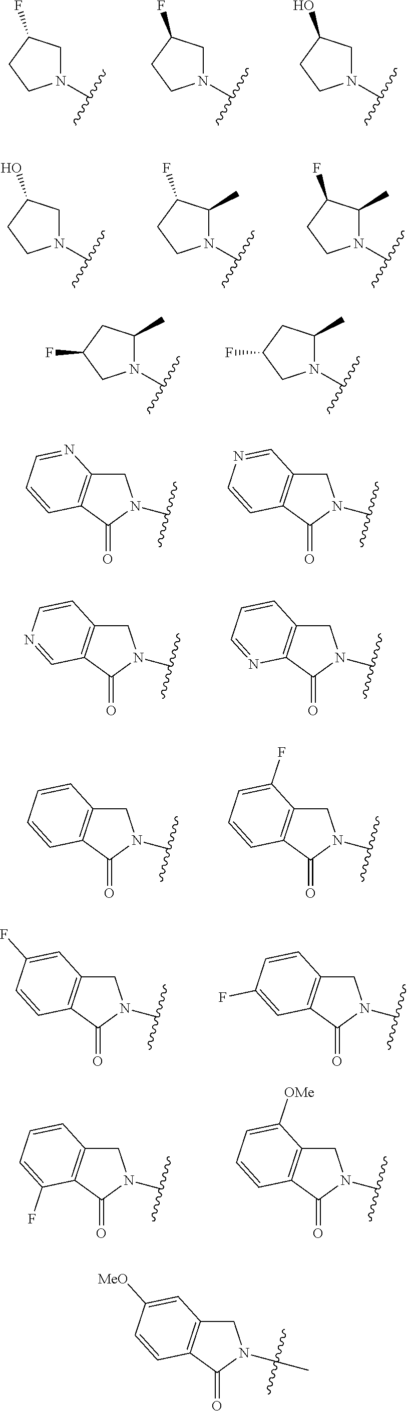

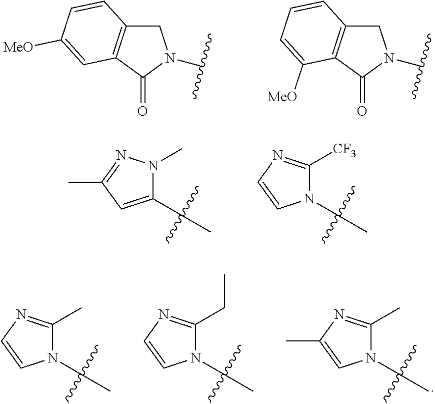

In some embodiments, R₂ and R₃ are both methyl.

In some embodiments, W is CR^{a}R^{b}, and R^{a} and R^{b} are each independently selected from H, halo, C₁-C₆ alkyl, OH or C₃-C₆ cycloalkyl.

In some embodiments, R₄ is selected from the group consisting of:

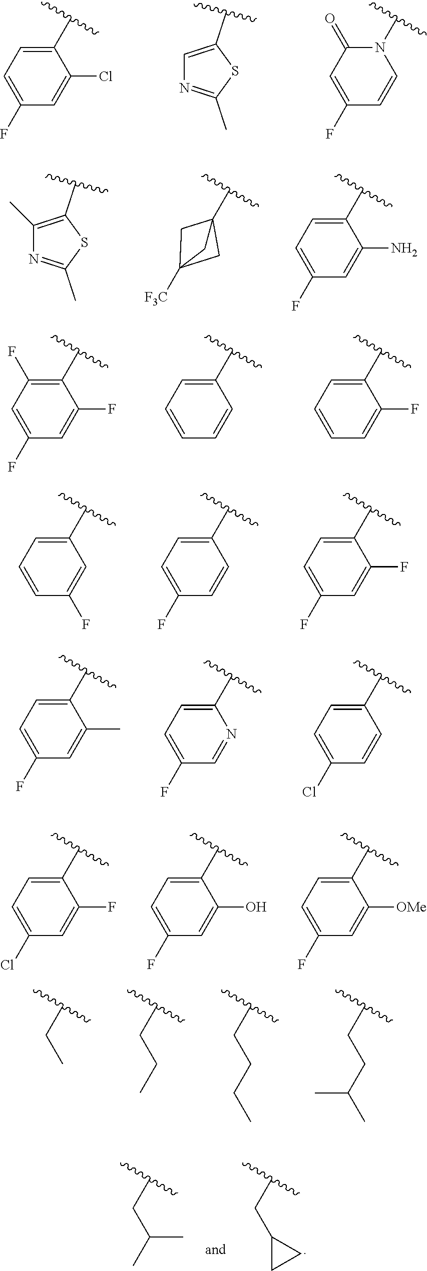

In some embodiments, R₄ is selected from the group consisting of:

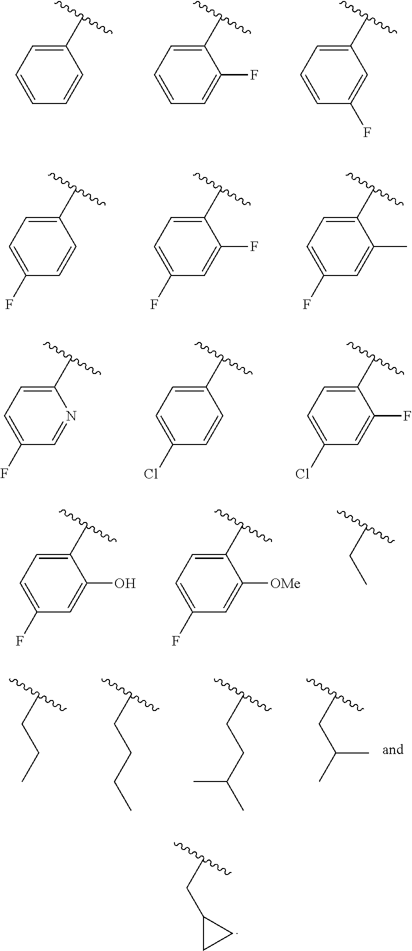

In some embodiments, wherein —W—R₄ is a moiety selected from:

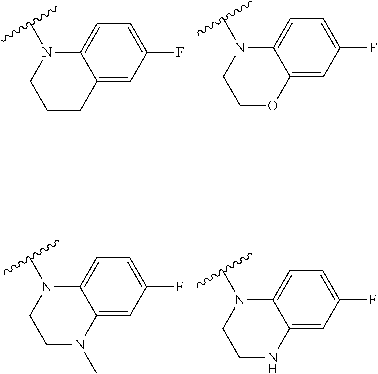

In some embodiments, R₅ and R₆ are both hydrogen.

In some embodiments, the halo is preferably selected from F or Cl.

In some embodiments, the C₁-C₆ alkyl is preferably selected from C₁-C₄ alkyl, more preferably from methyl or ethyl.

In some embodiments, for the above aspects of the disclosure, the compound is selected from the group consisting of:

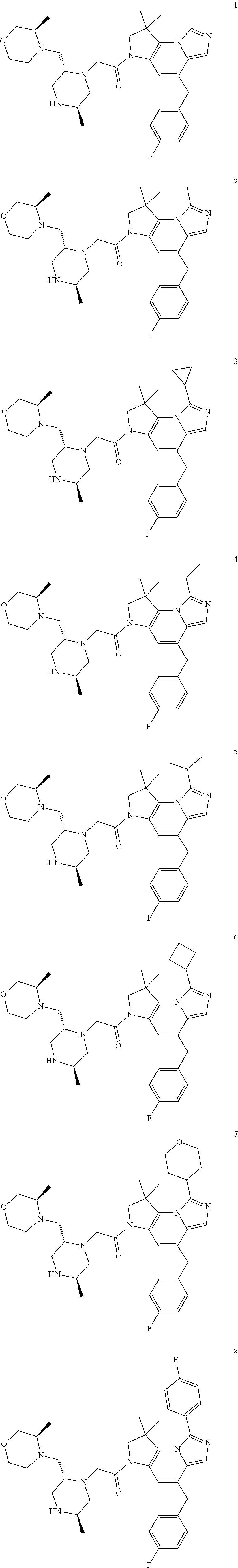

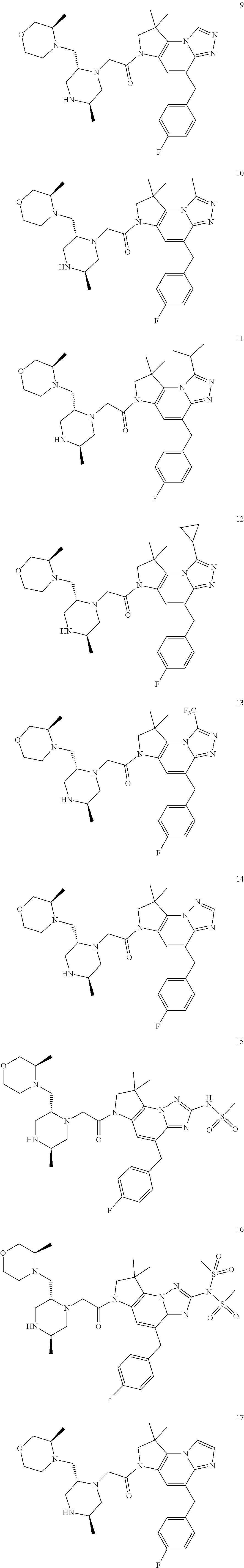

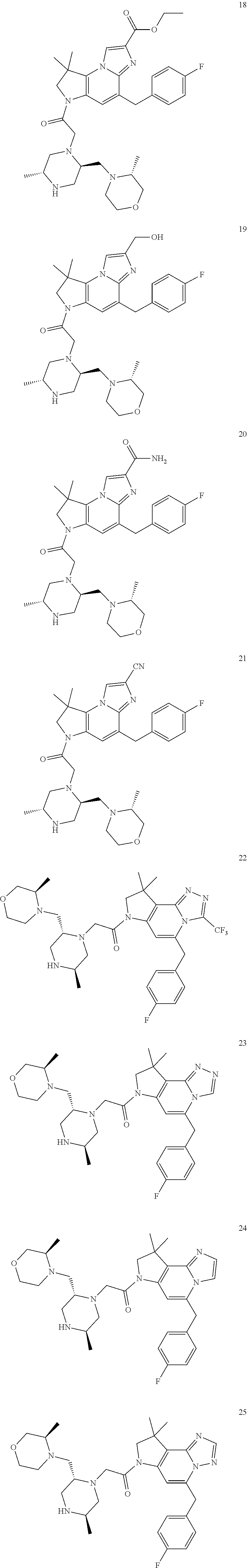

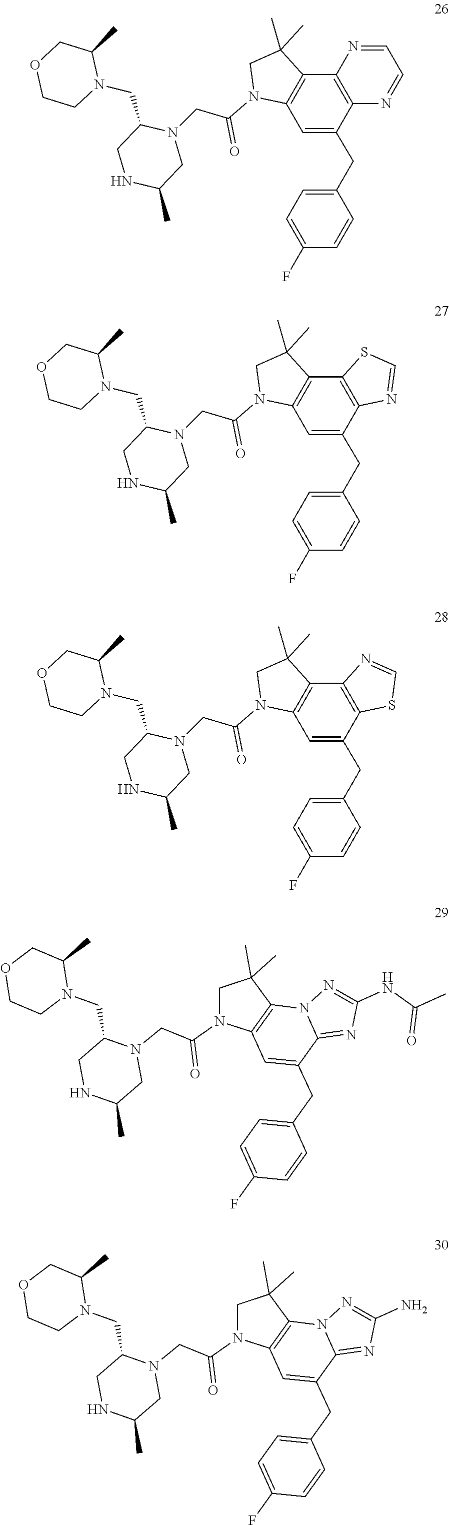

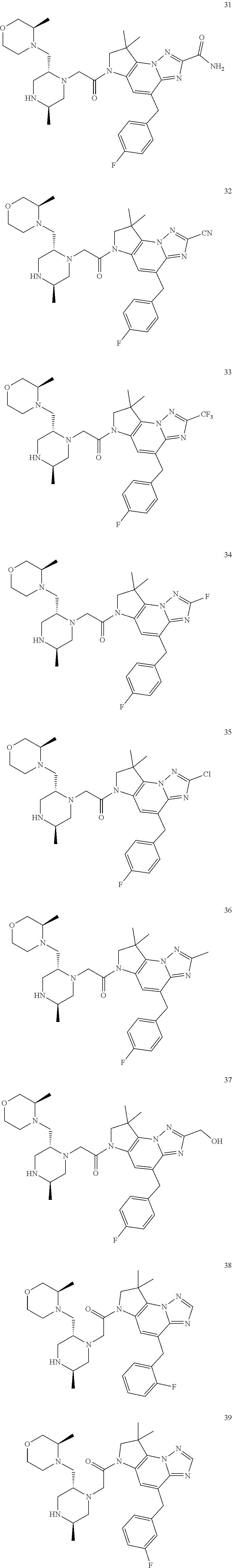

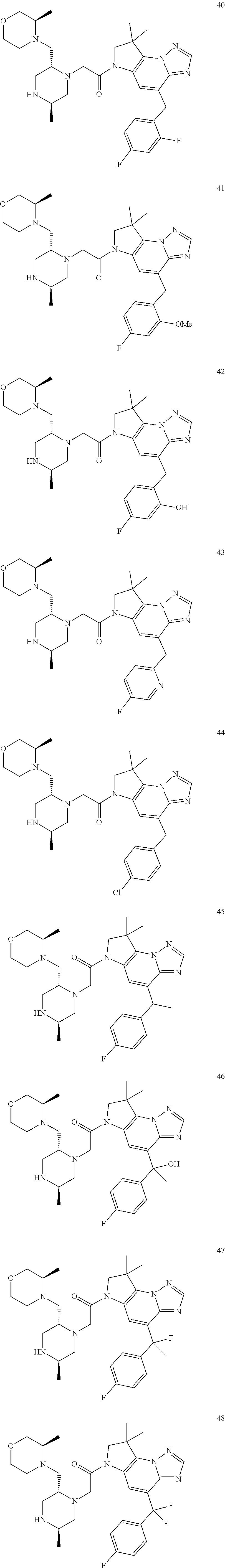

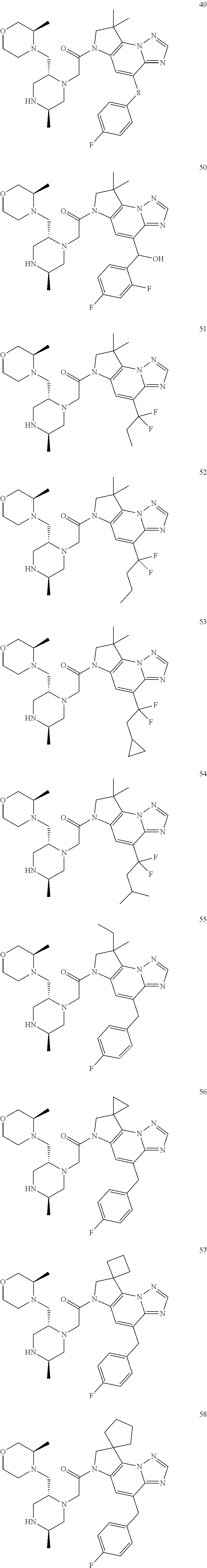

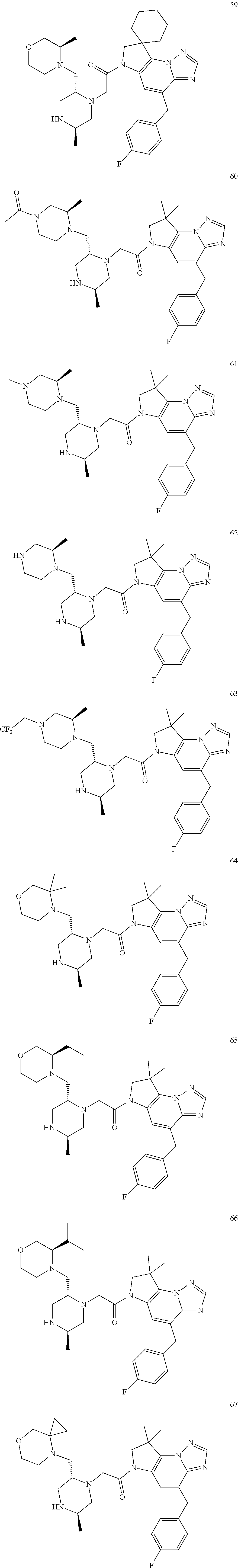

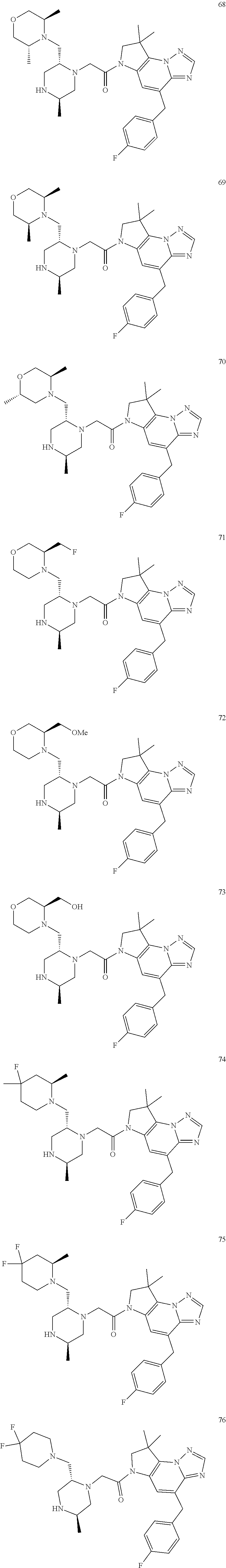

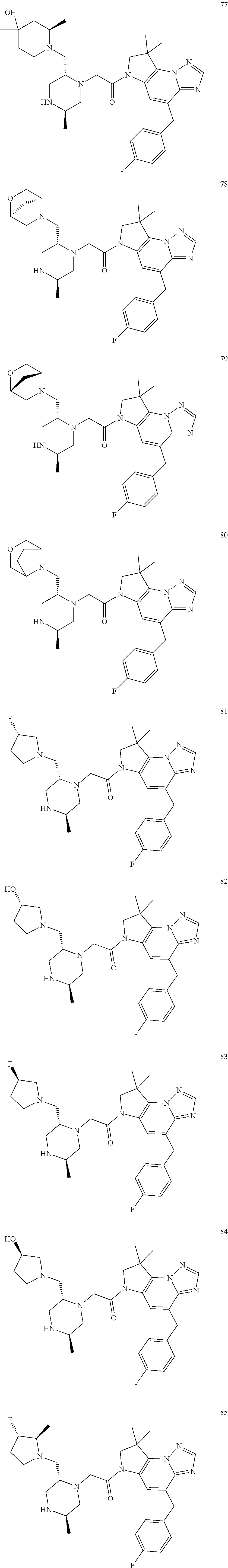

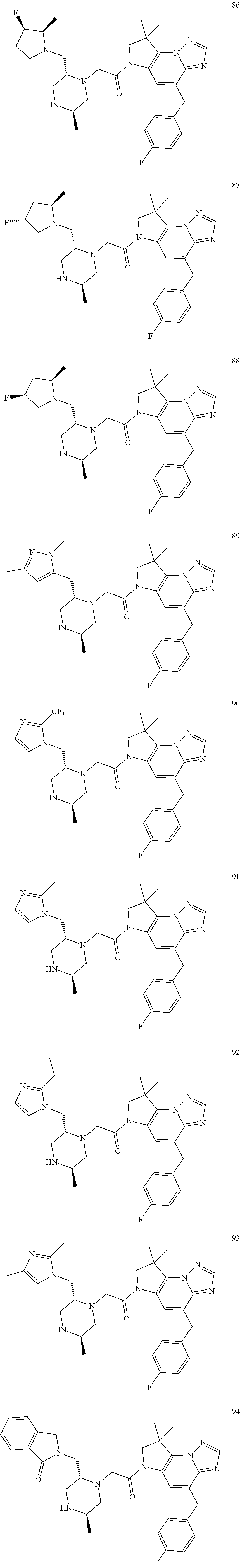

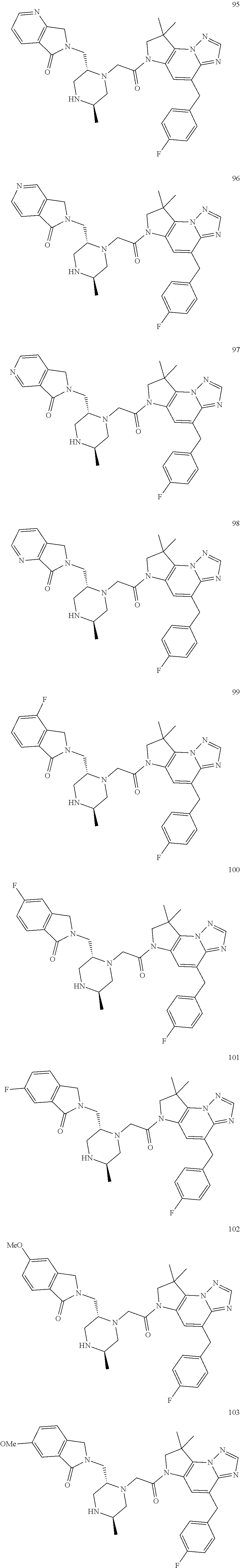

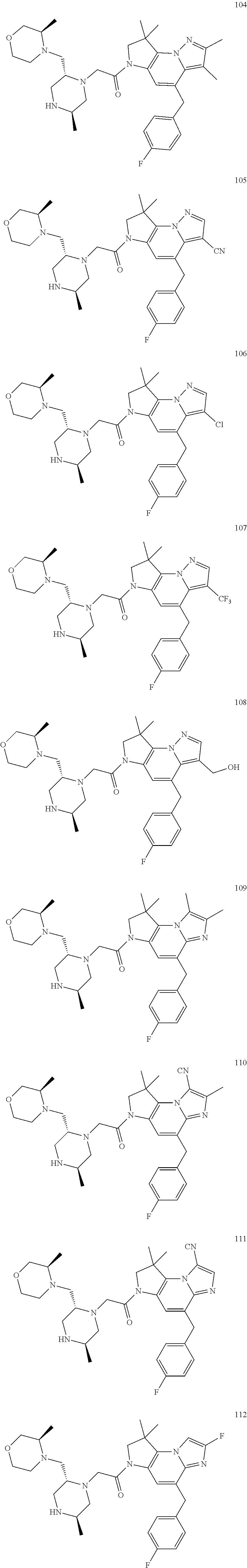

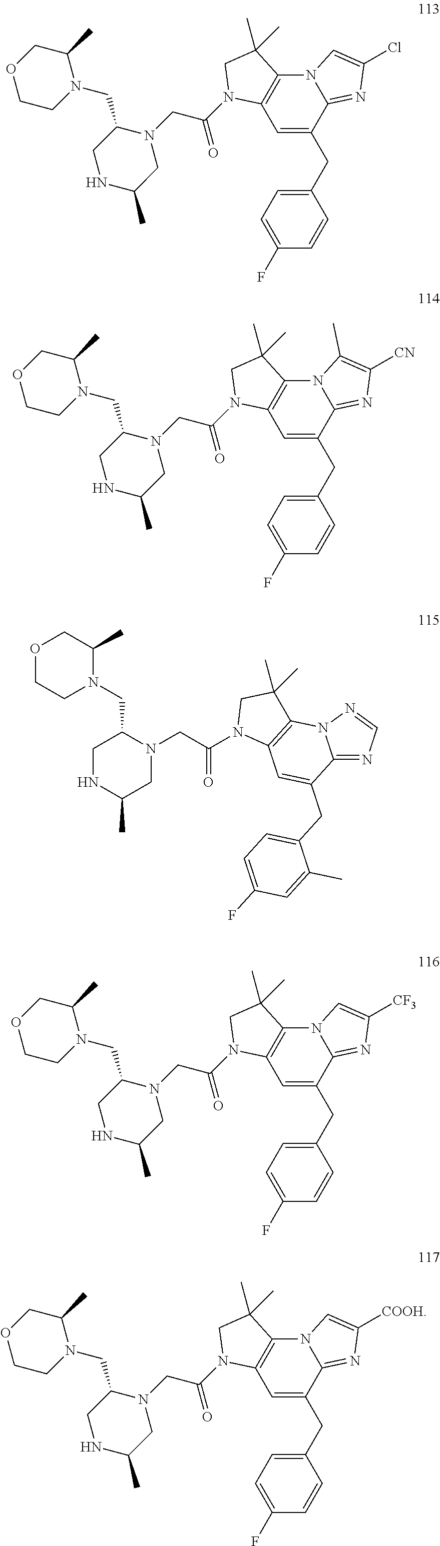 [SMILES: C[C@@H]1CN(CC(=O)N2CC(C)(C)c3c2cc(Cc2ccc(F)cc2)c2nc(Cl)cn32)[C@@H](CN2CCOC[C@H]2C)CN1 ; Cc1c(C#N)nc2c(Cc3ccc(F)cc3)cc3c(n12)C(C)(C)CN3C(=O)CN1C[C@@H](C)NC[C@@H]1CN1CCOC[C@H]1C ; Cc1cc(F)ccc1Cc1cc2c(n3ncnc13)C(C)(C)CN2C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2nc(C(F)(F)F)cn32)[C@@H](CN2CCOC[C@H]2C)CN1 ; C[C@@H]1CN(CC(=O)N2CC(C)(C)c3c2cc(Cc2ccc(F)cc2)c2nc(C(=O)O)cn32)[C@@H](CN2CCOC[C@H]2C)CN1]

or a pharmaceutically acceptable salt or solvate thereof.

In some embodiments, for the above aspects of the disclosure, the compound is selected from the group consisting of:

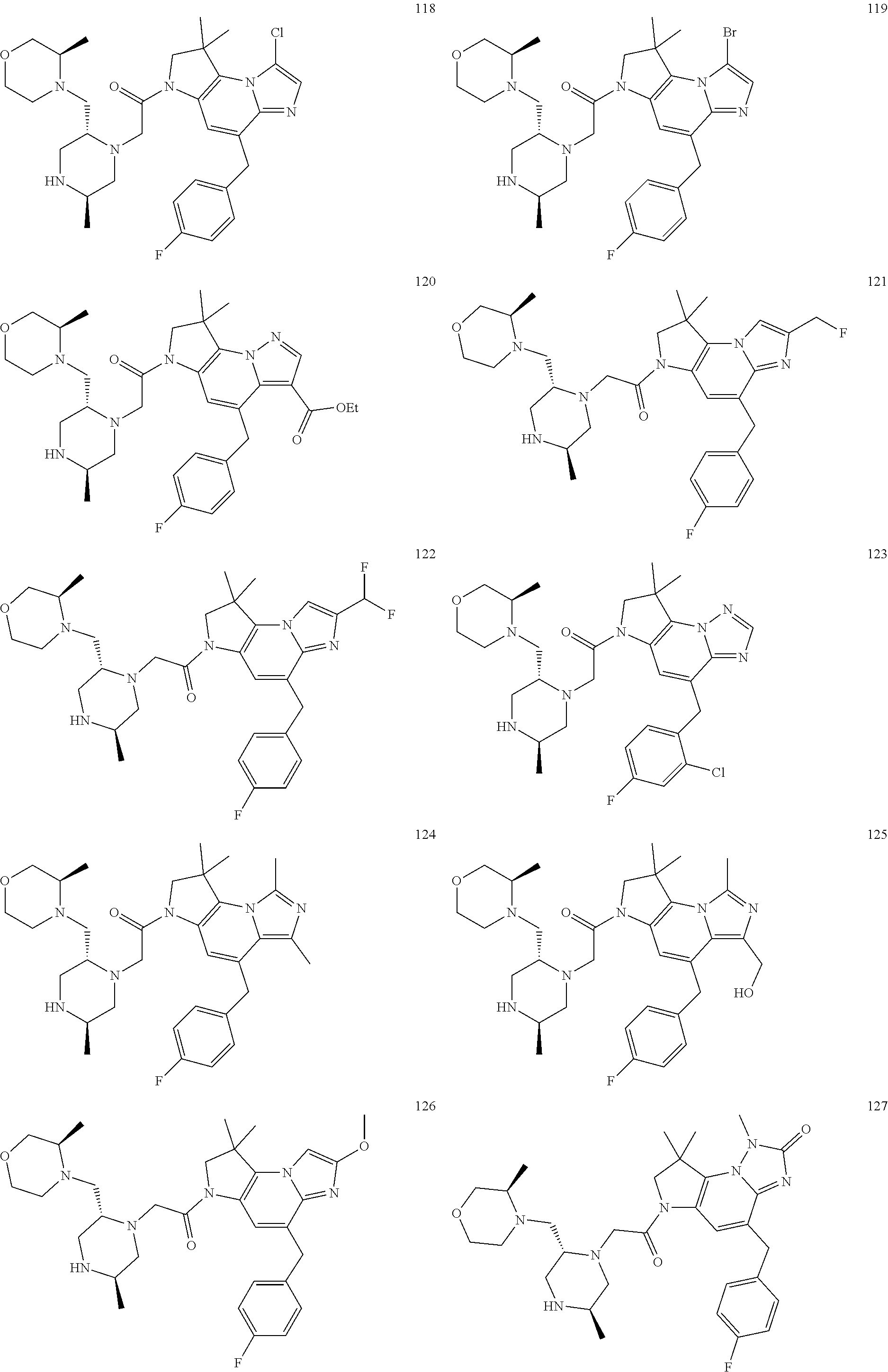

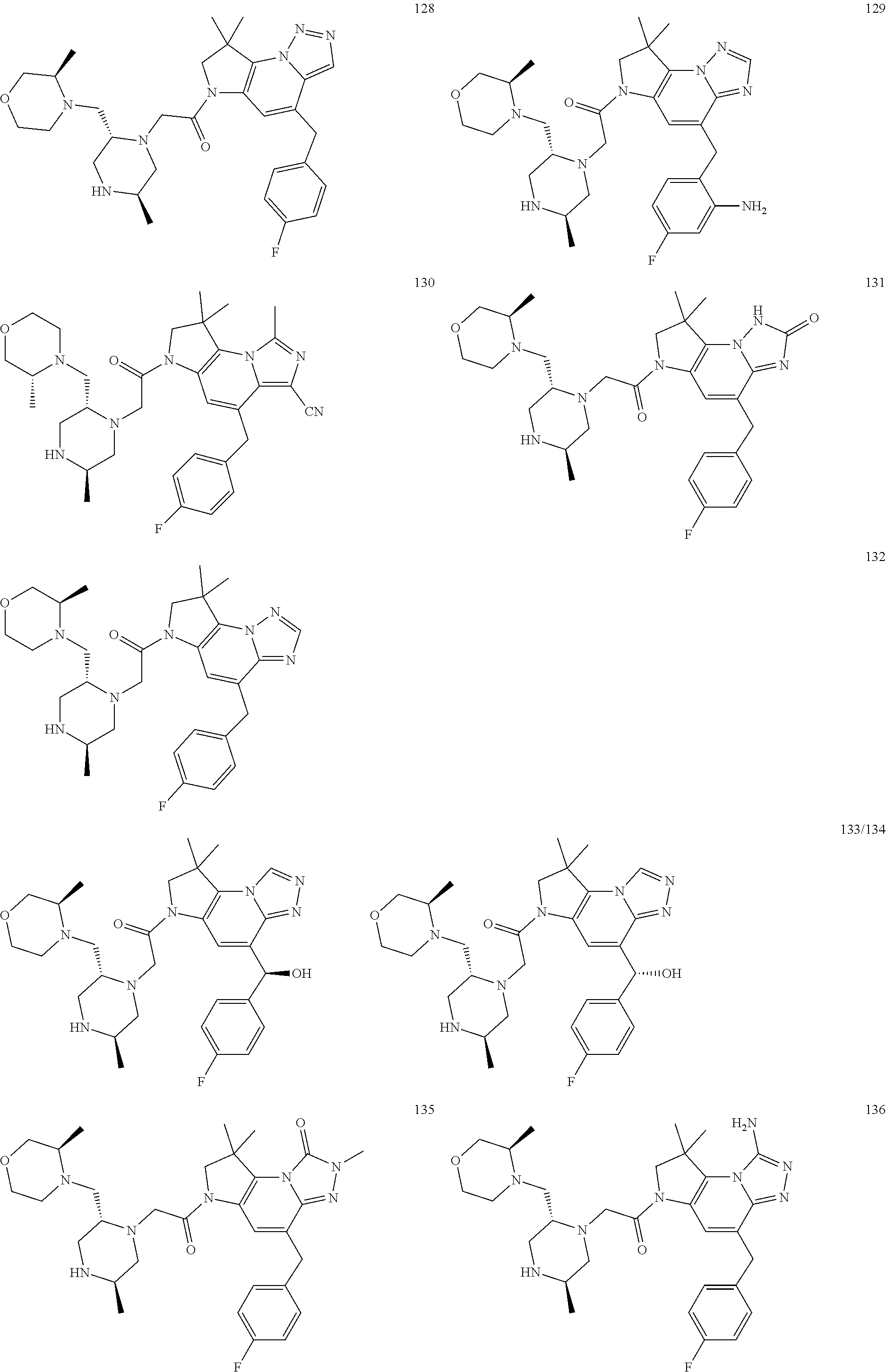

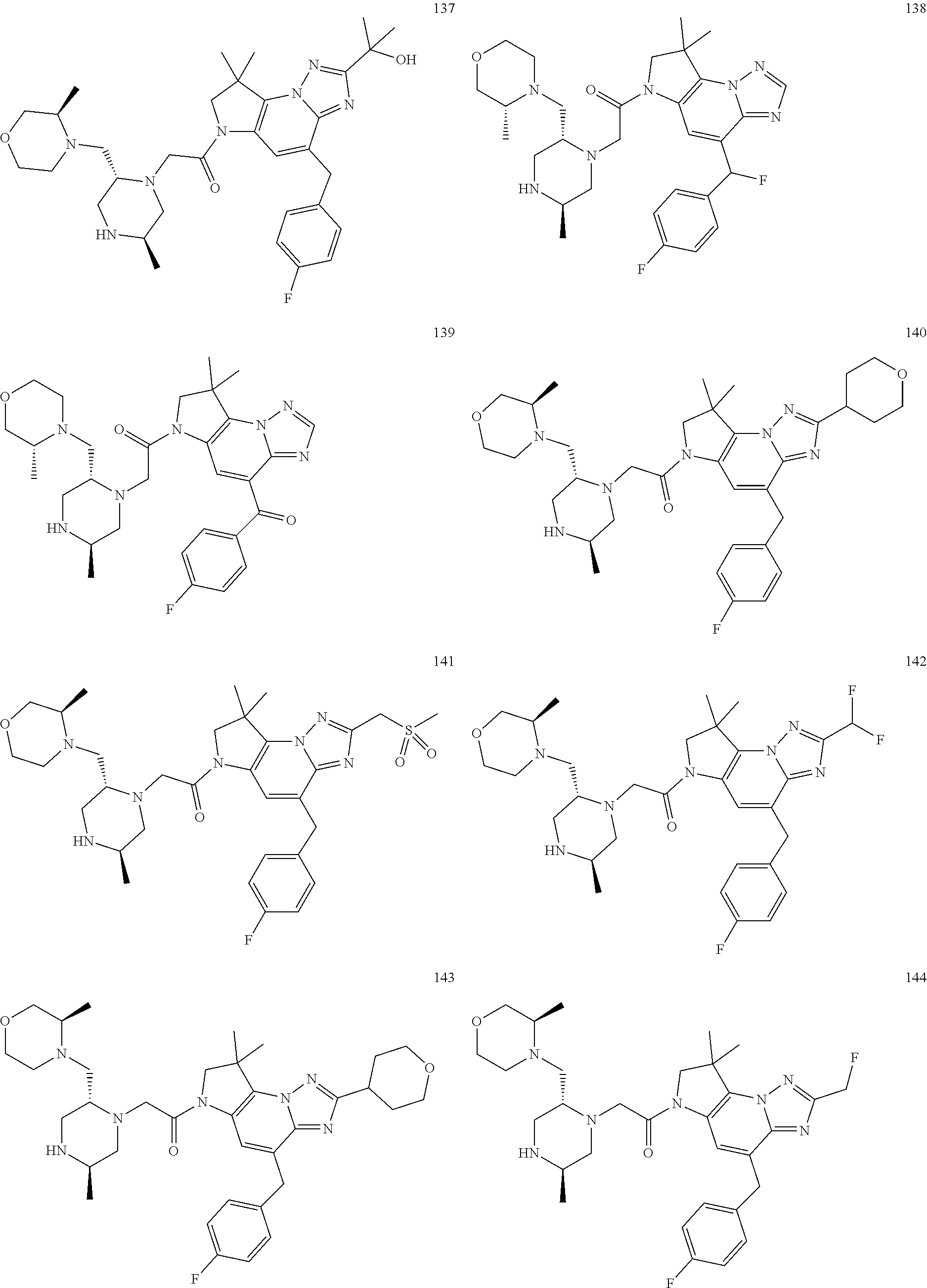

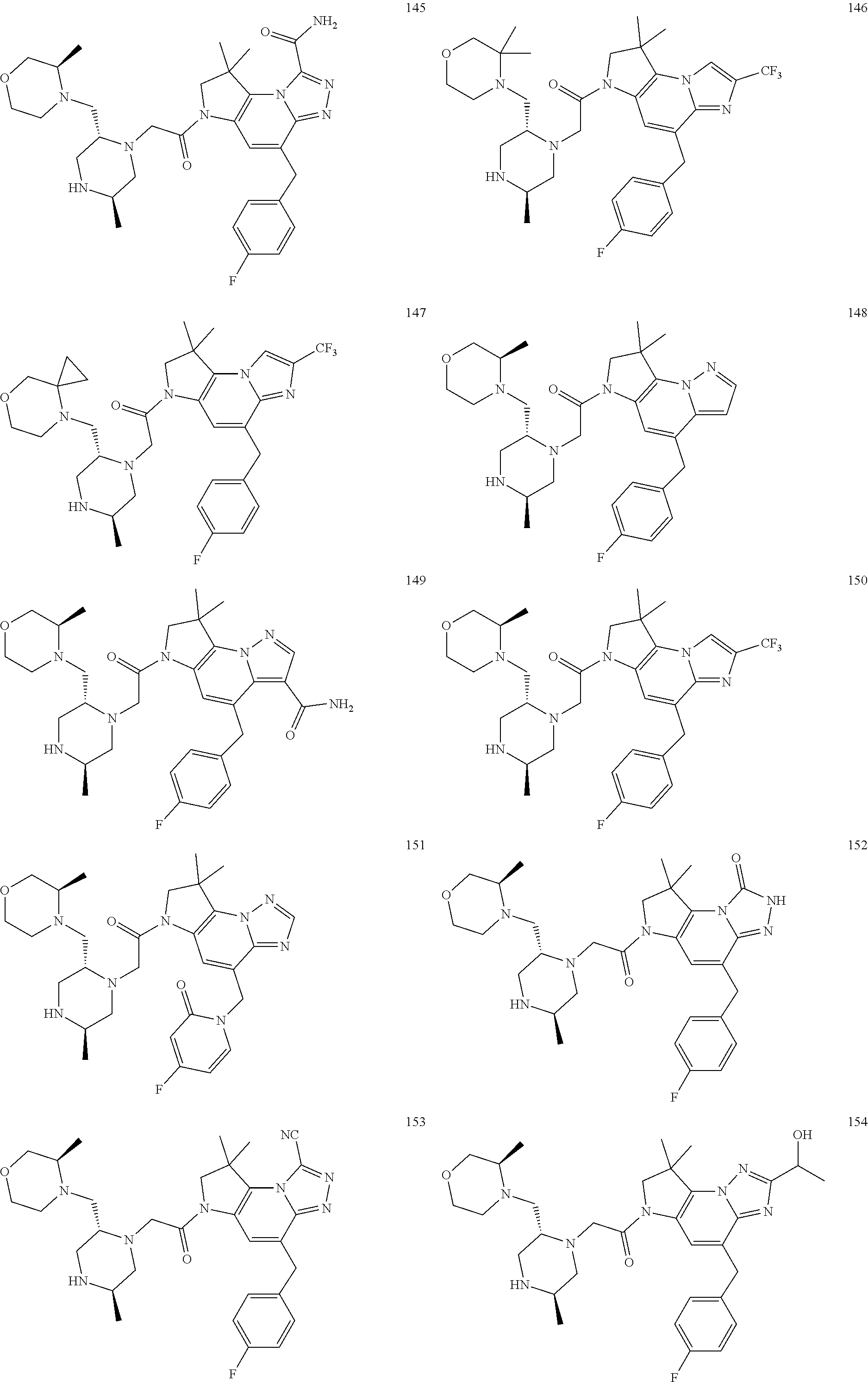

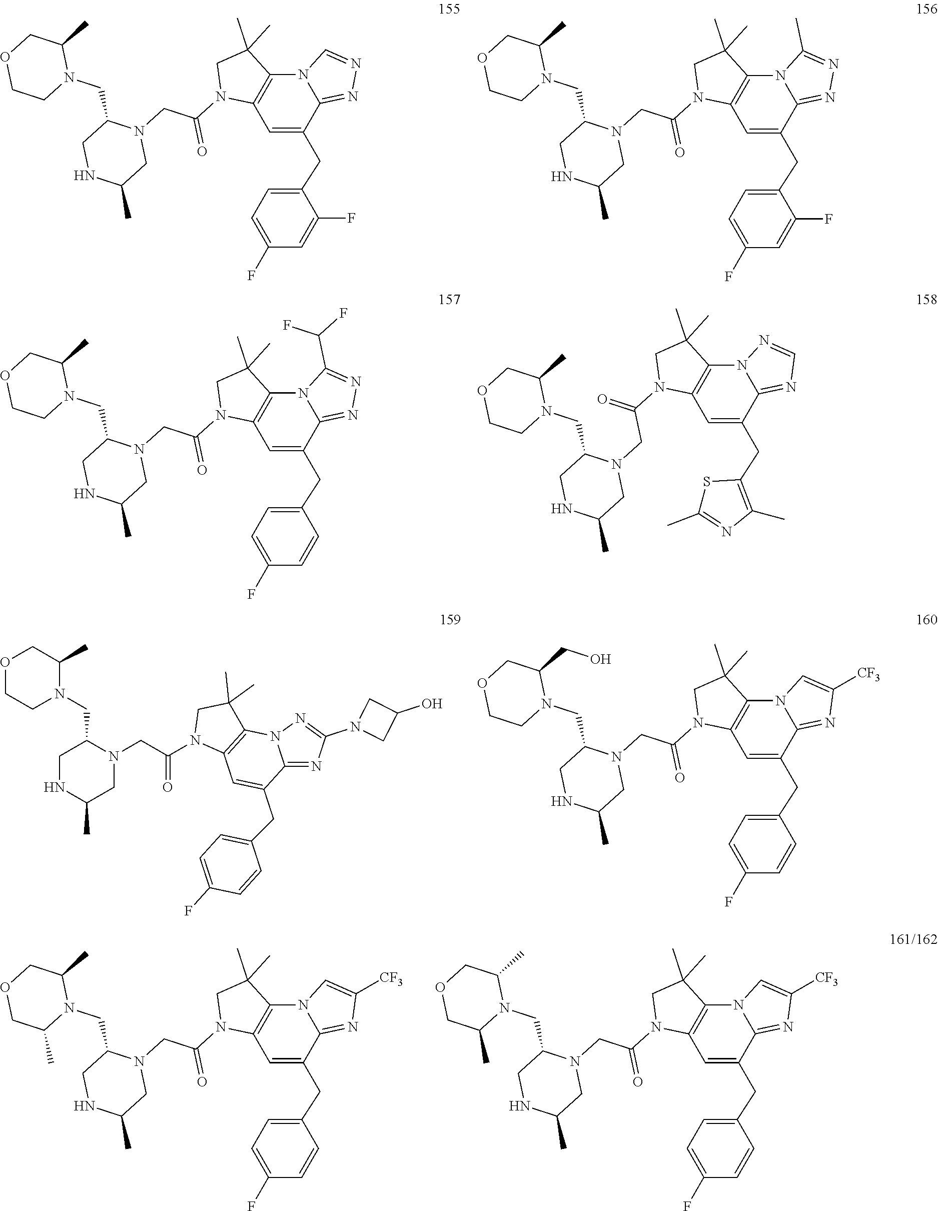

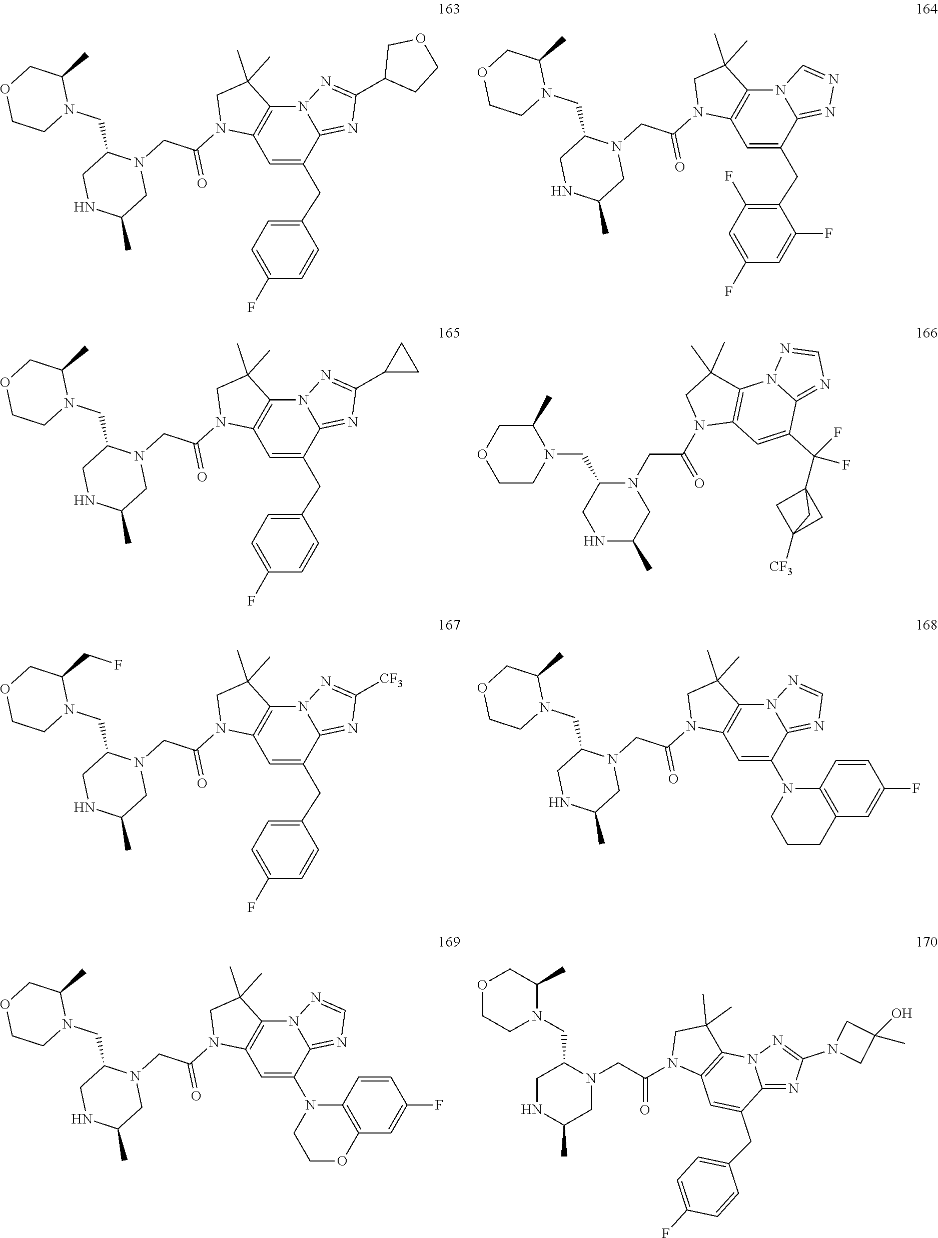

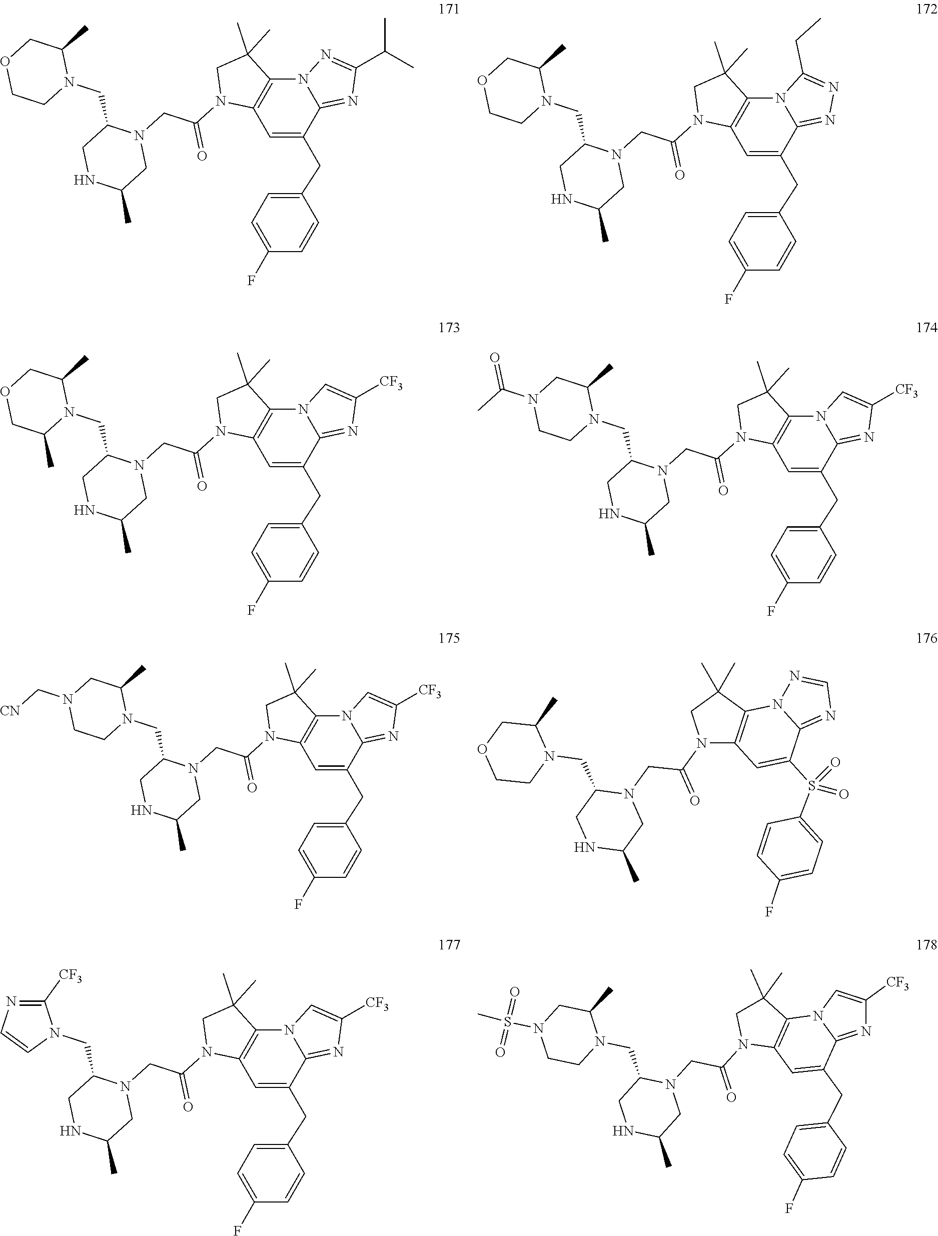

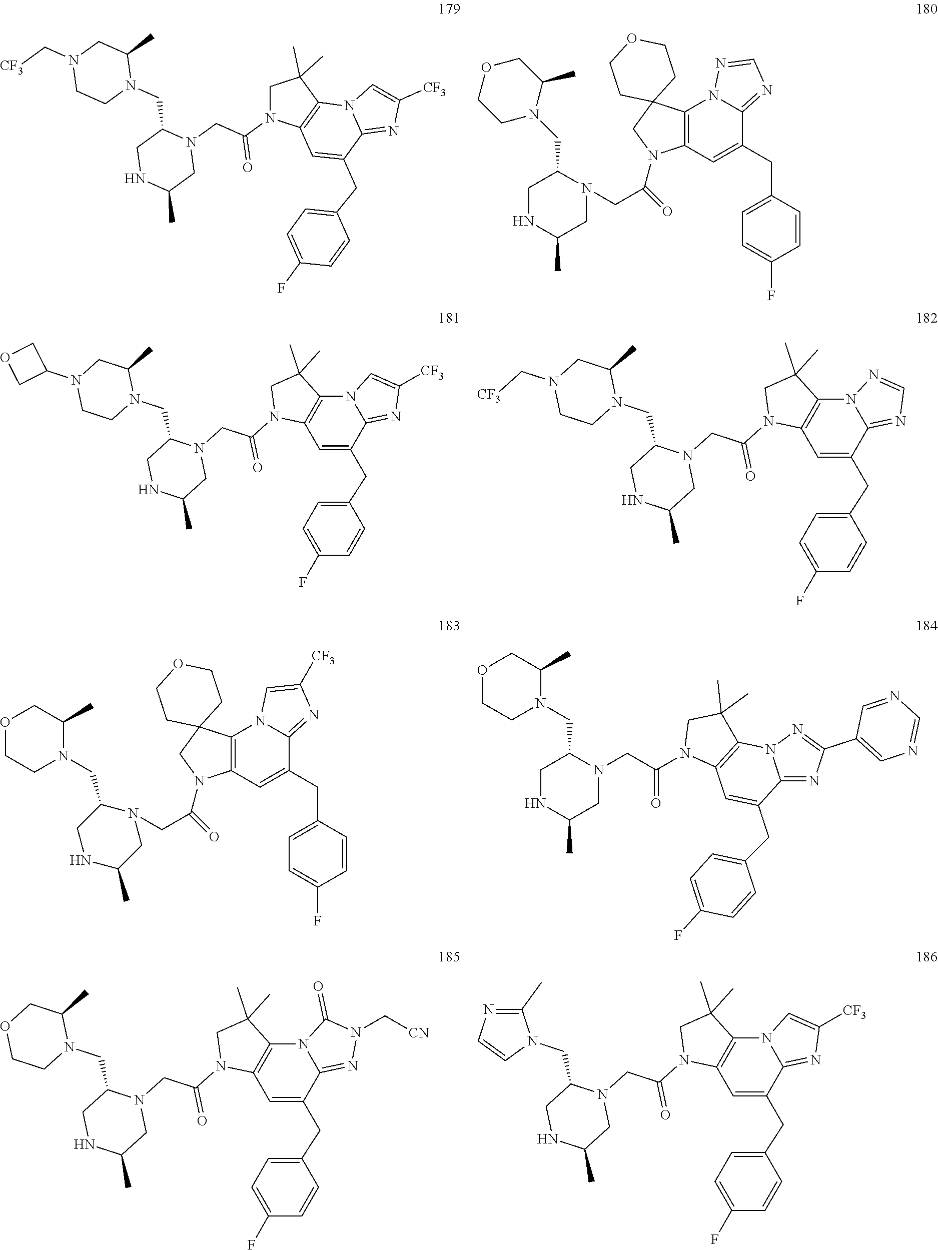

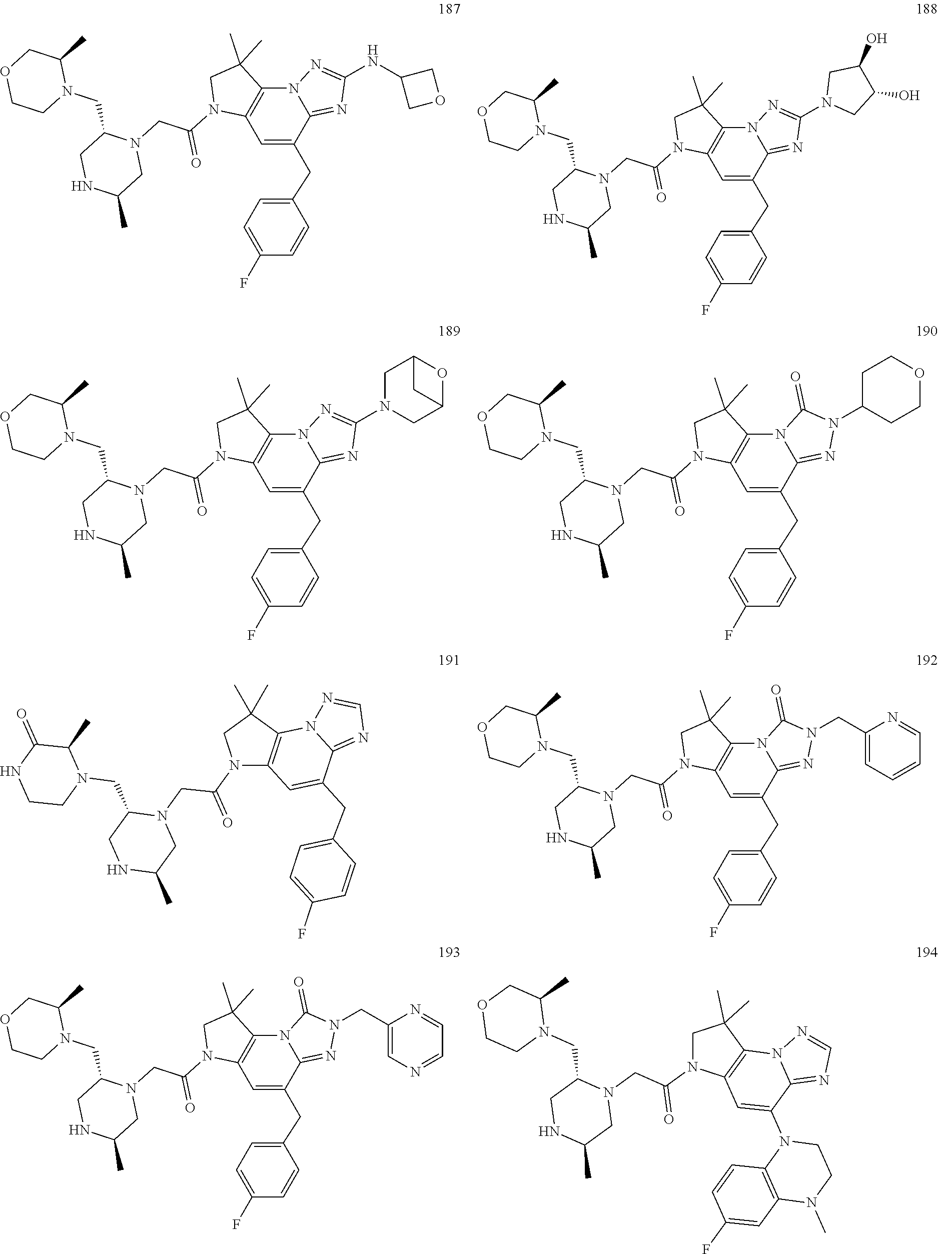

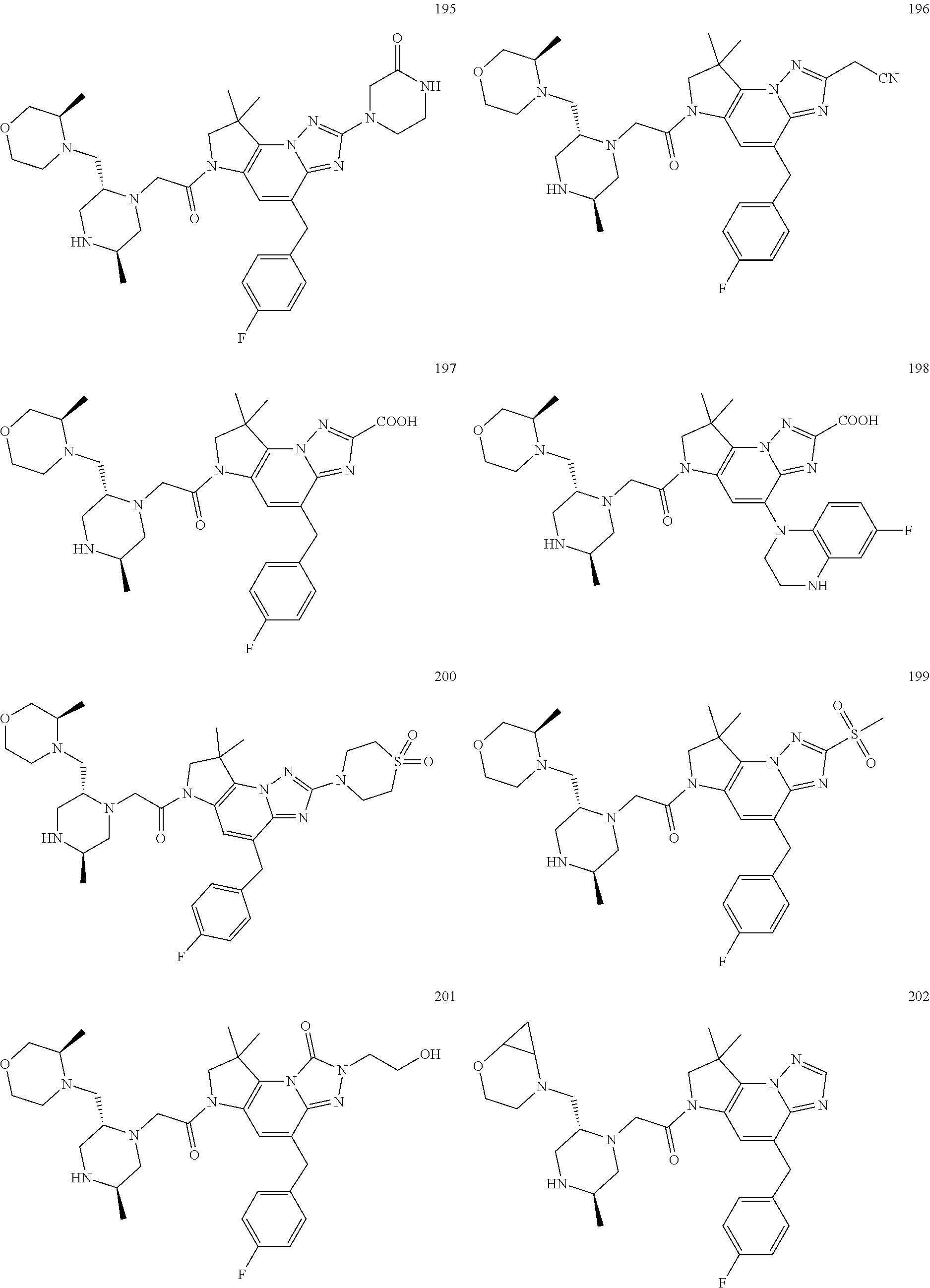

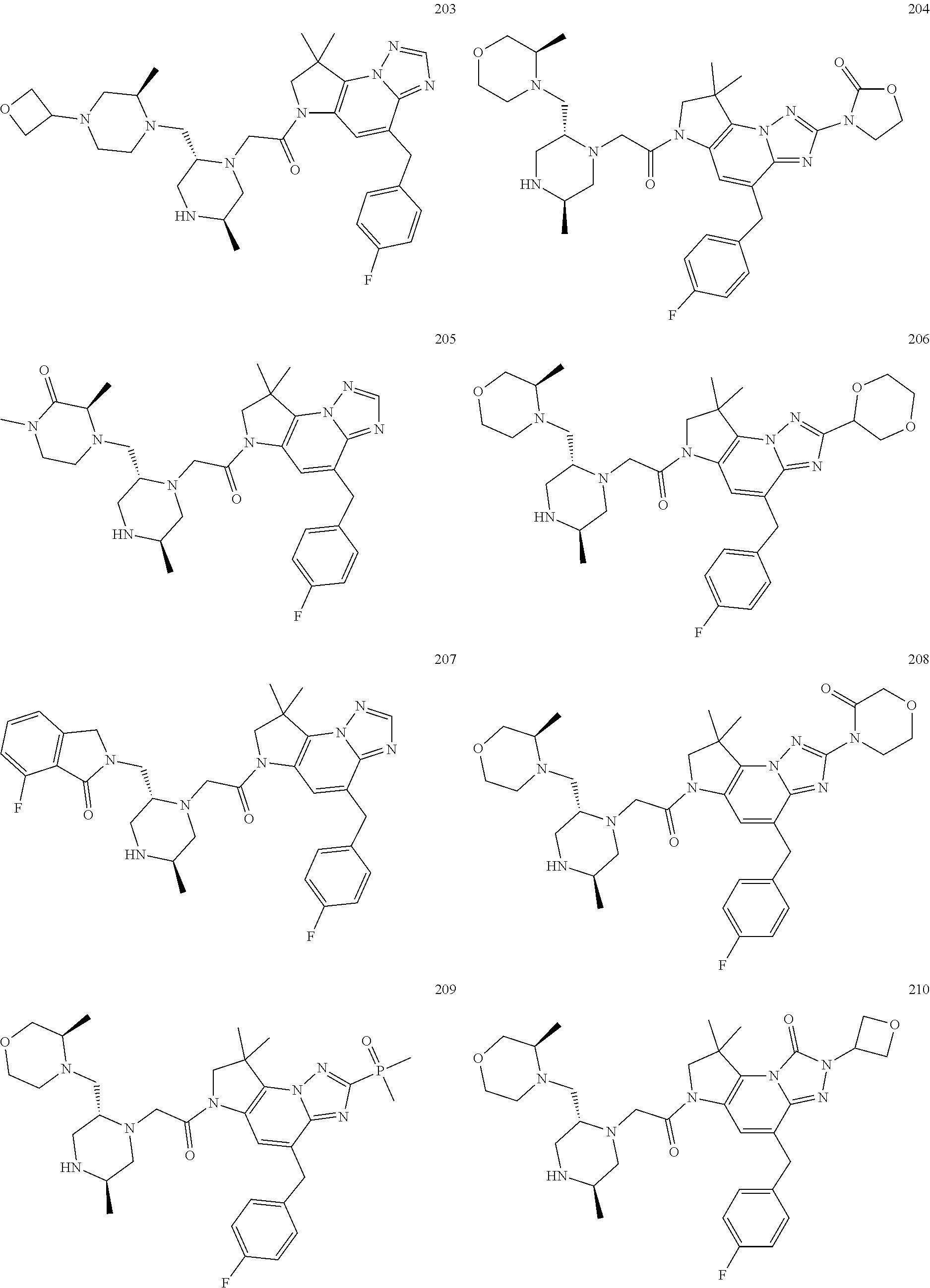

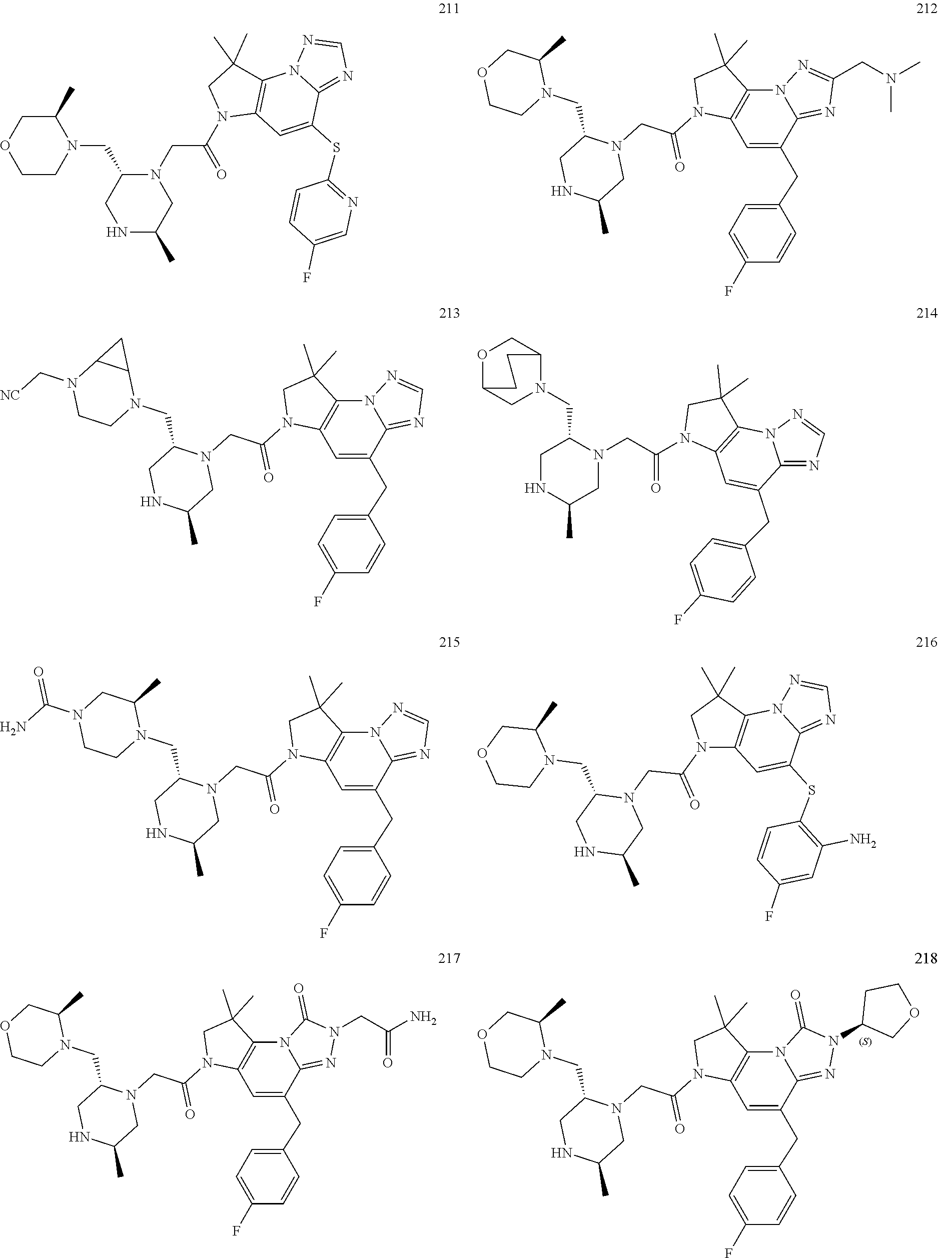

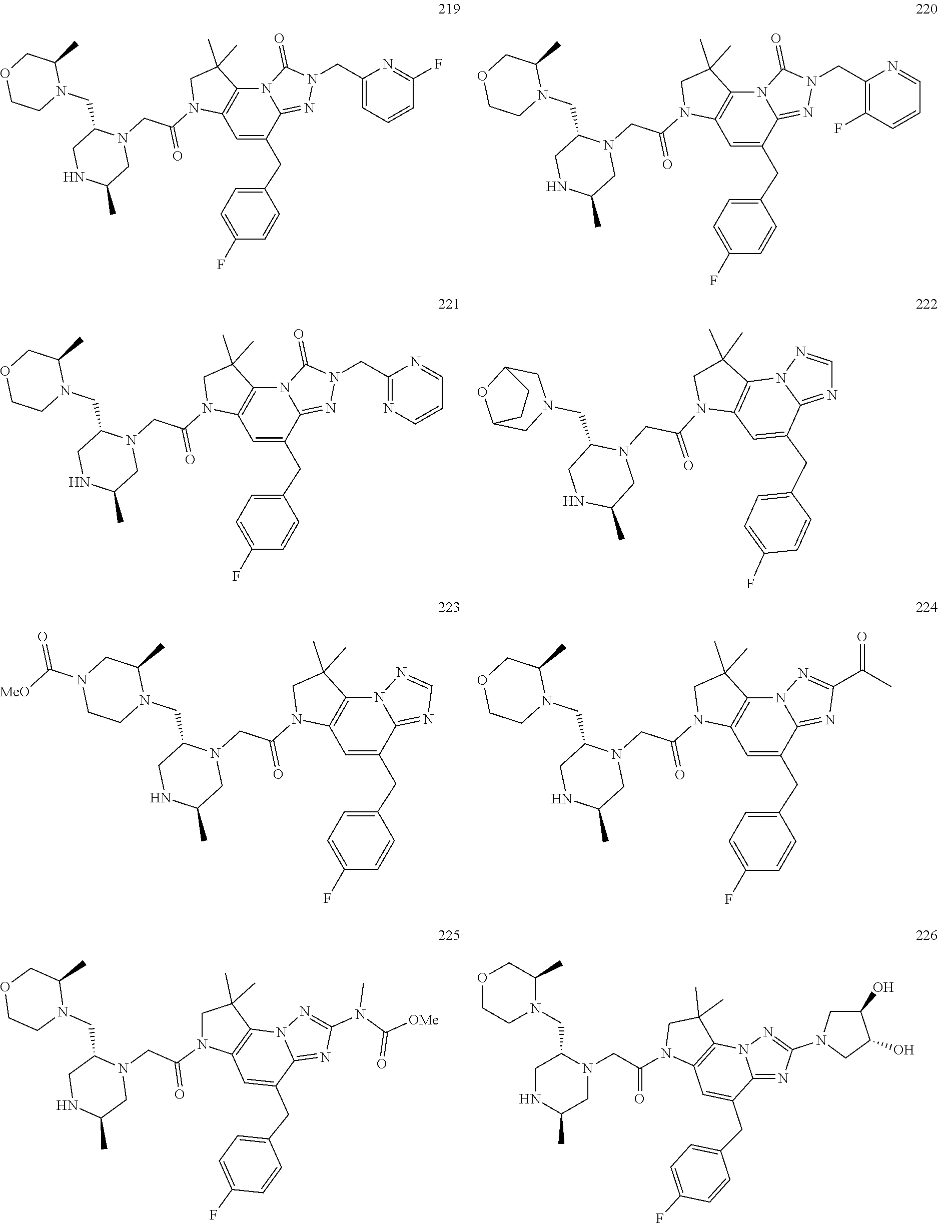

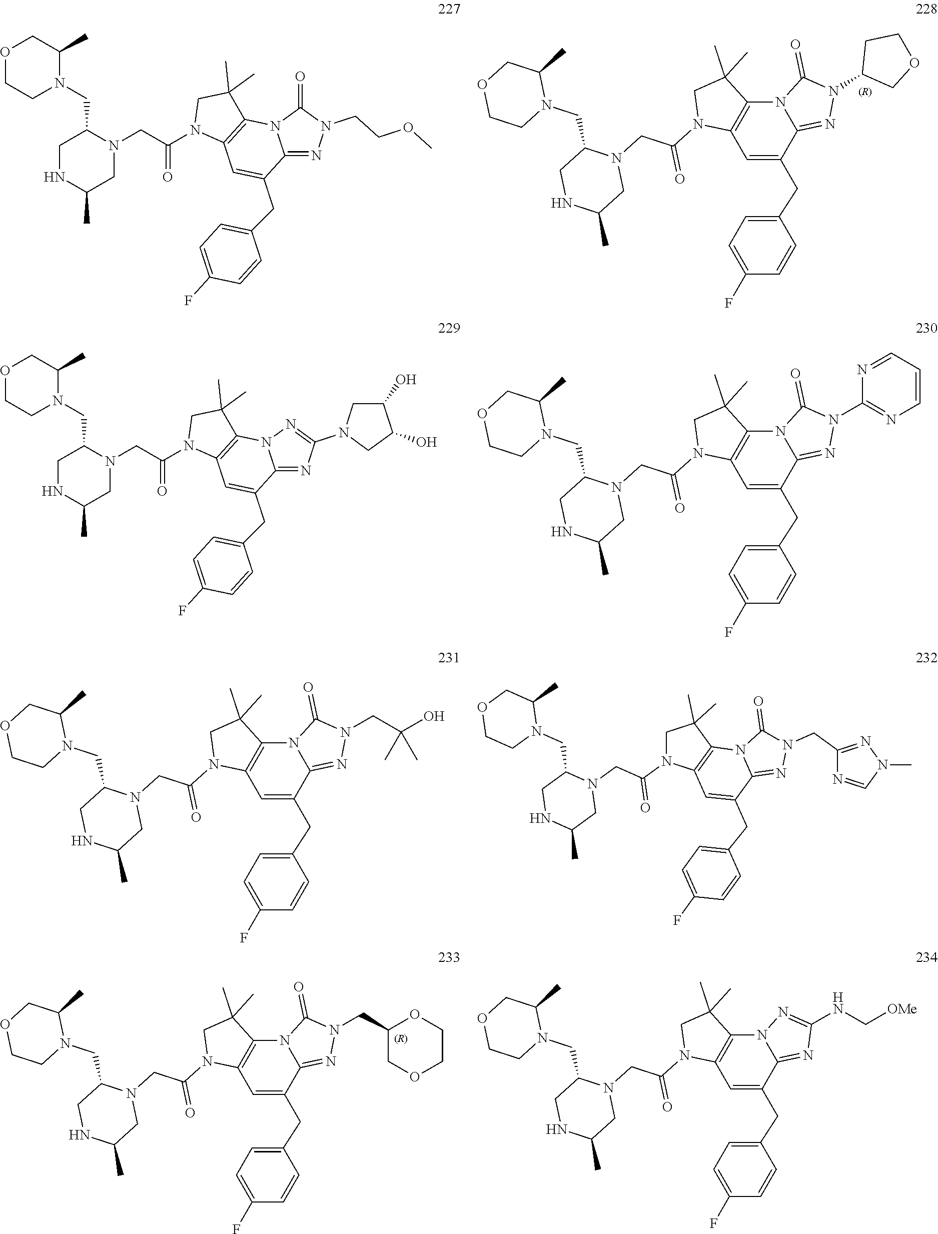

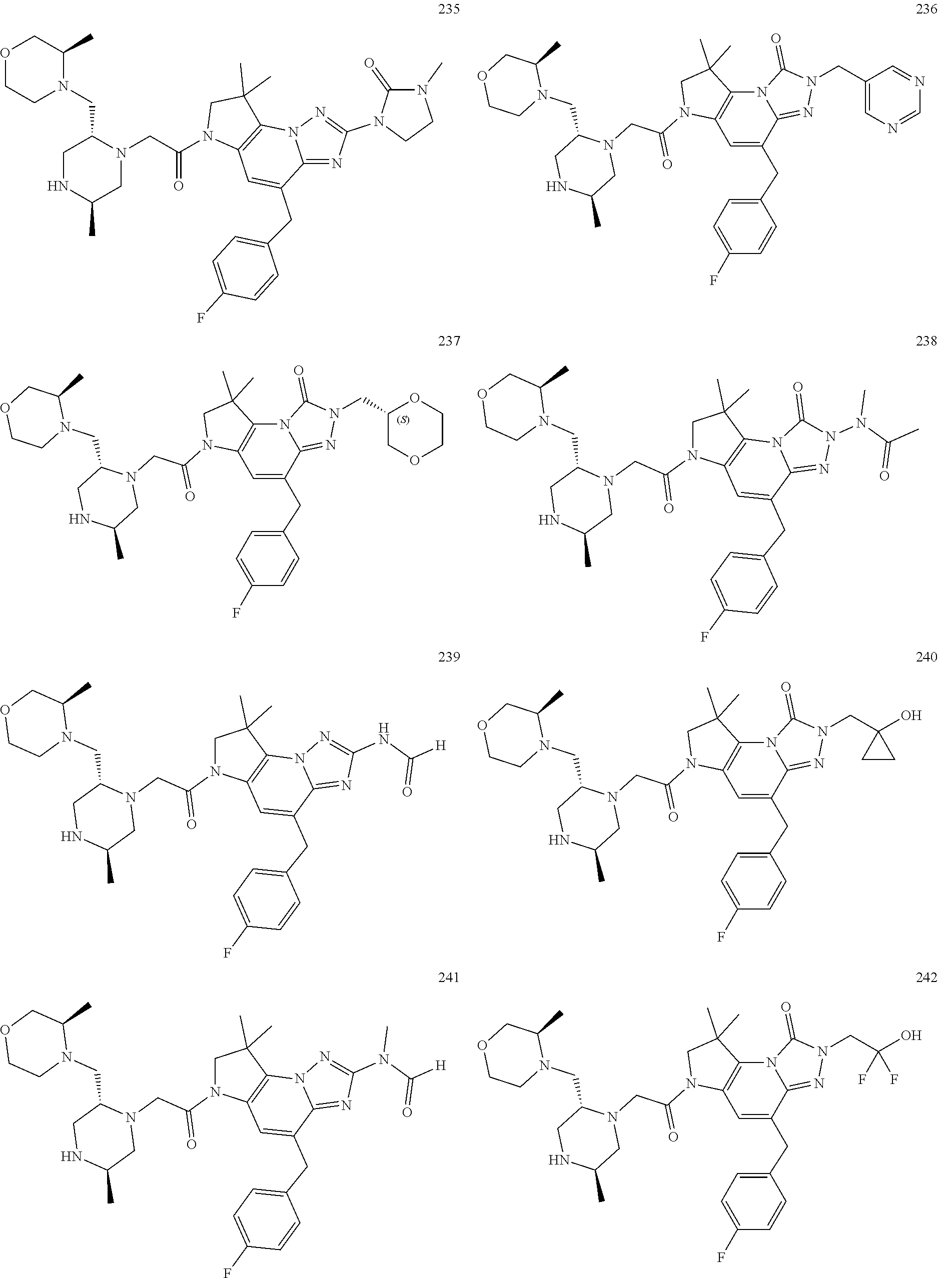

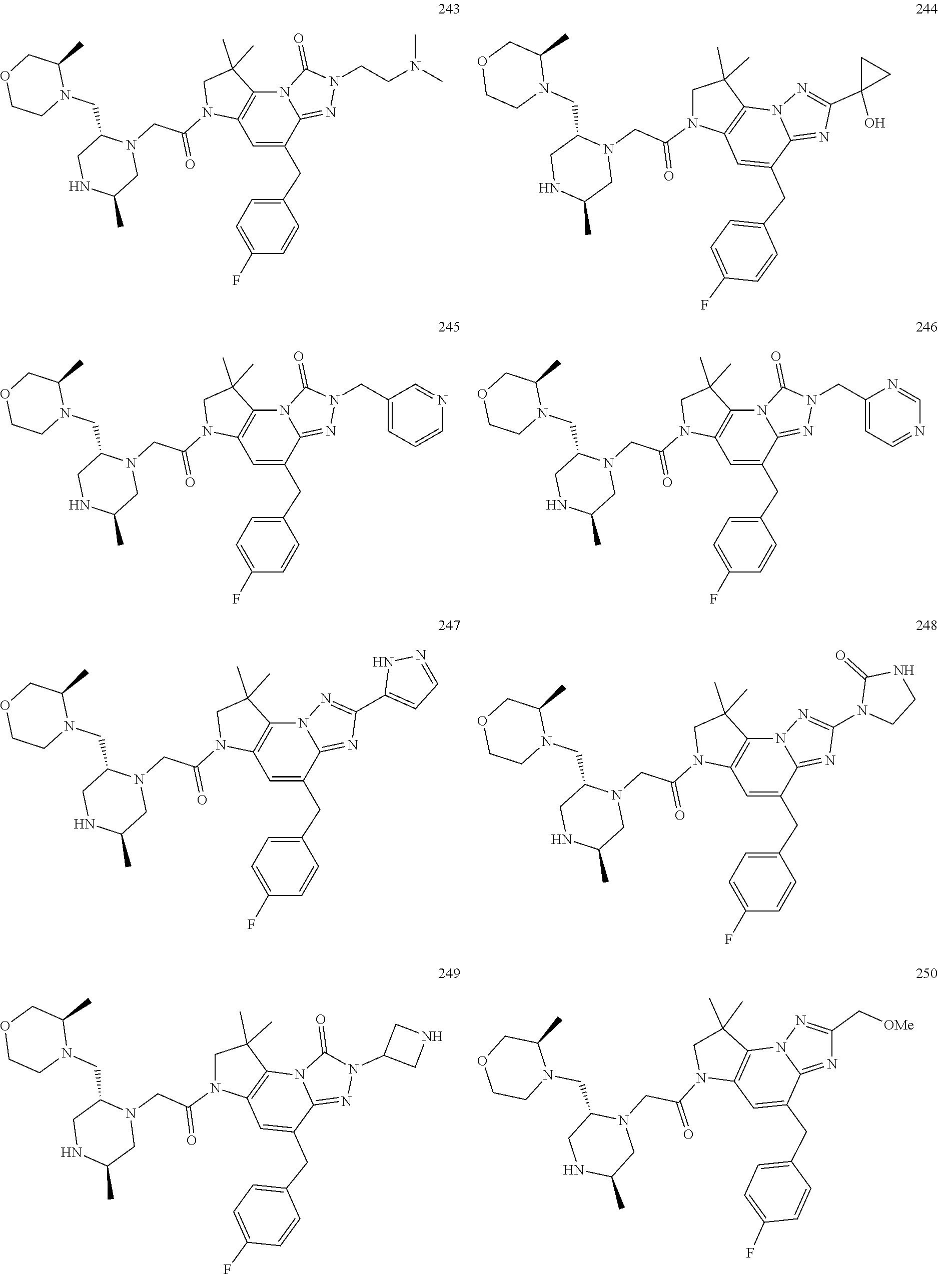

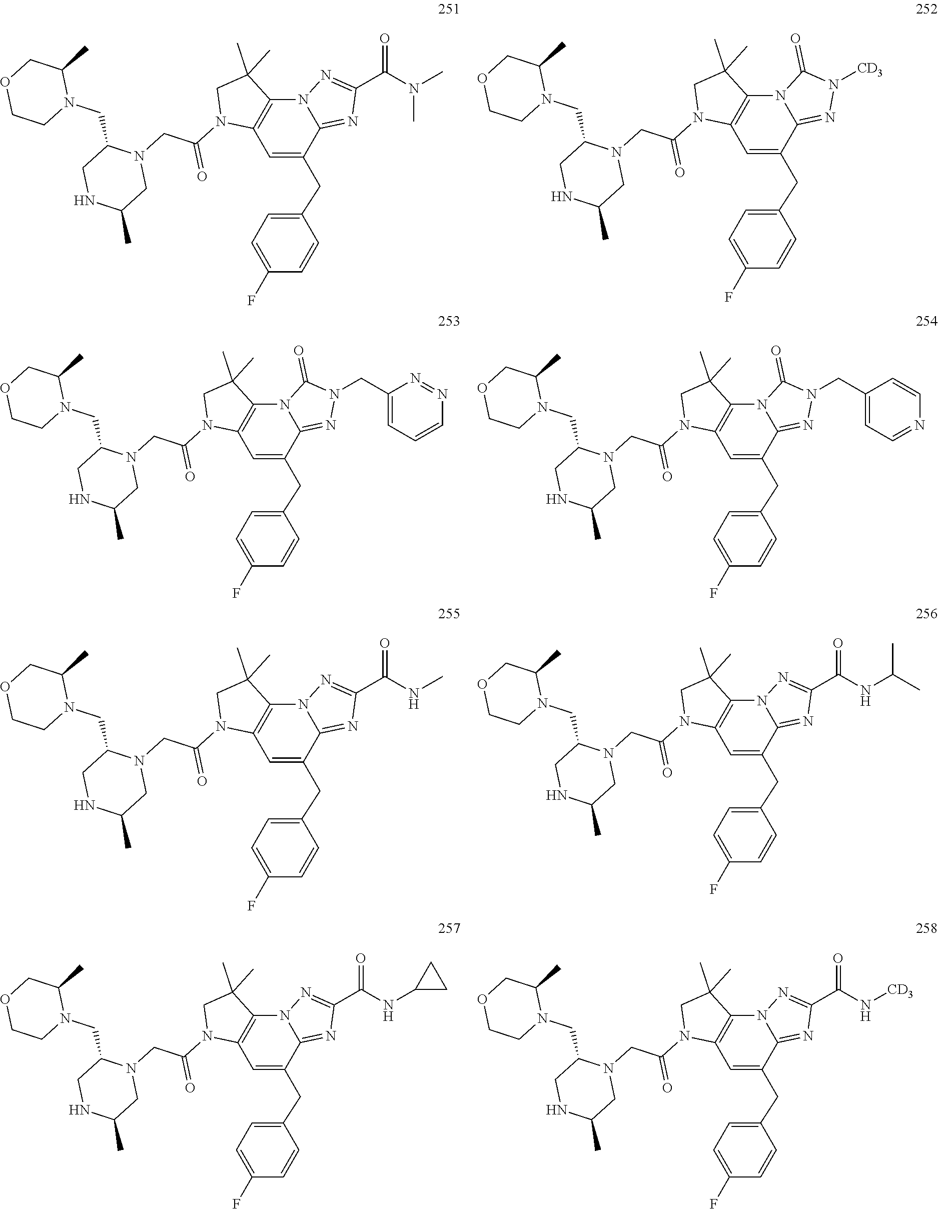

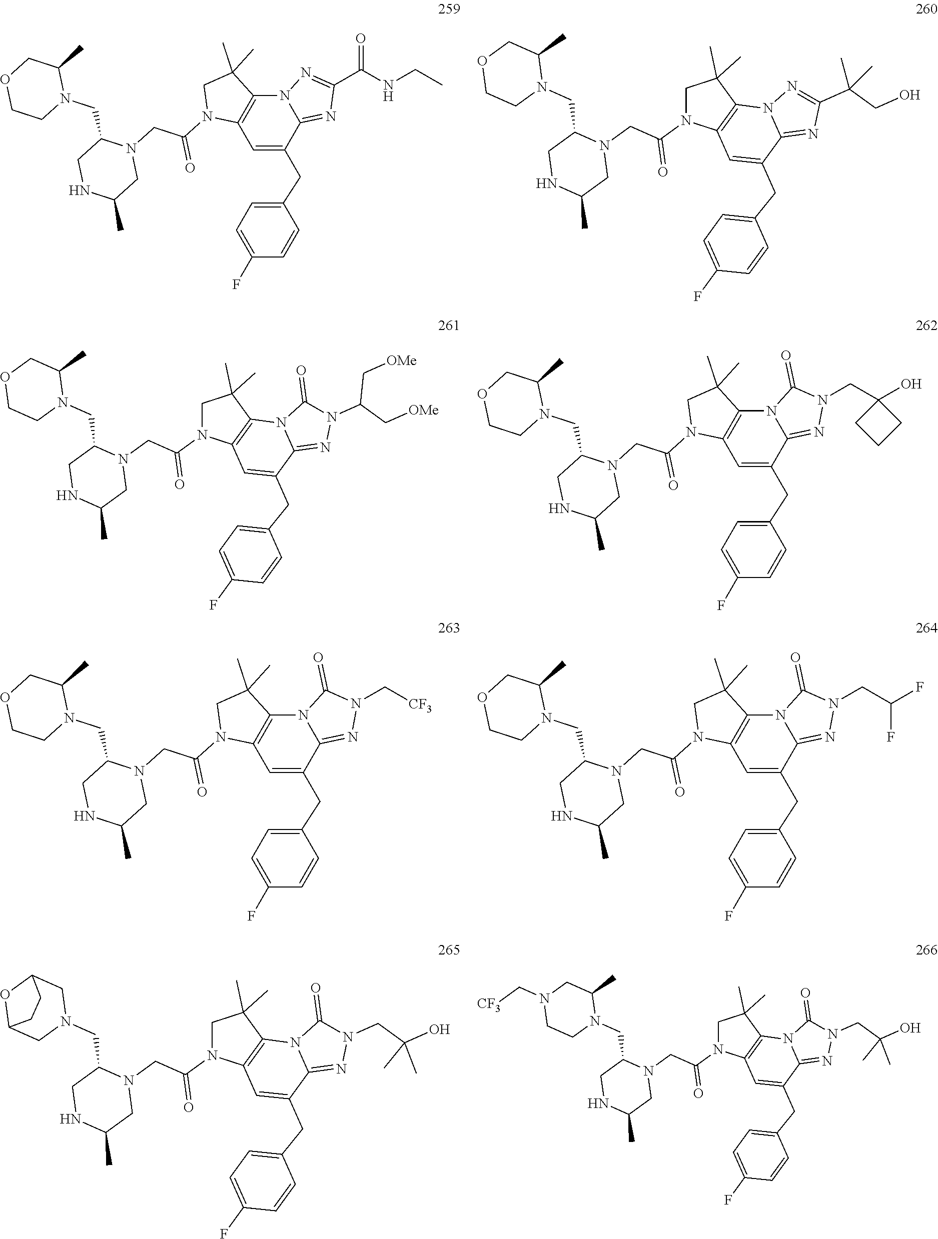

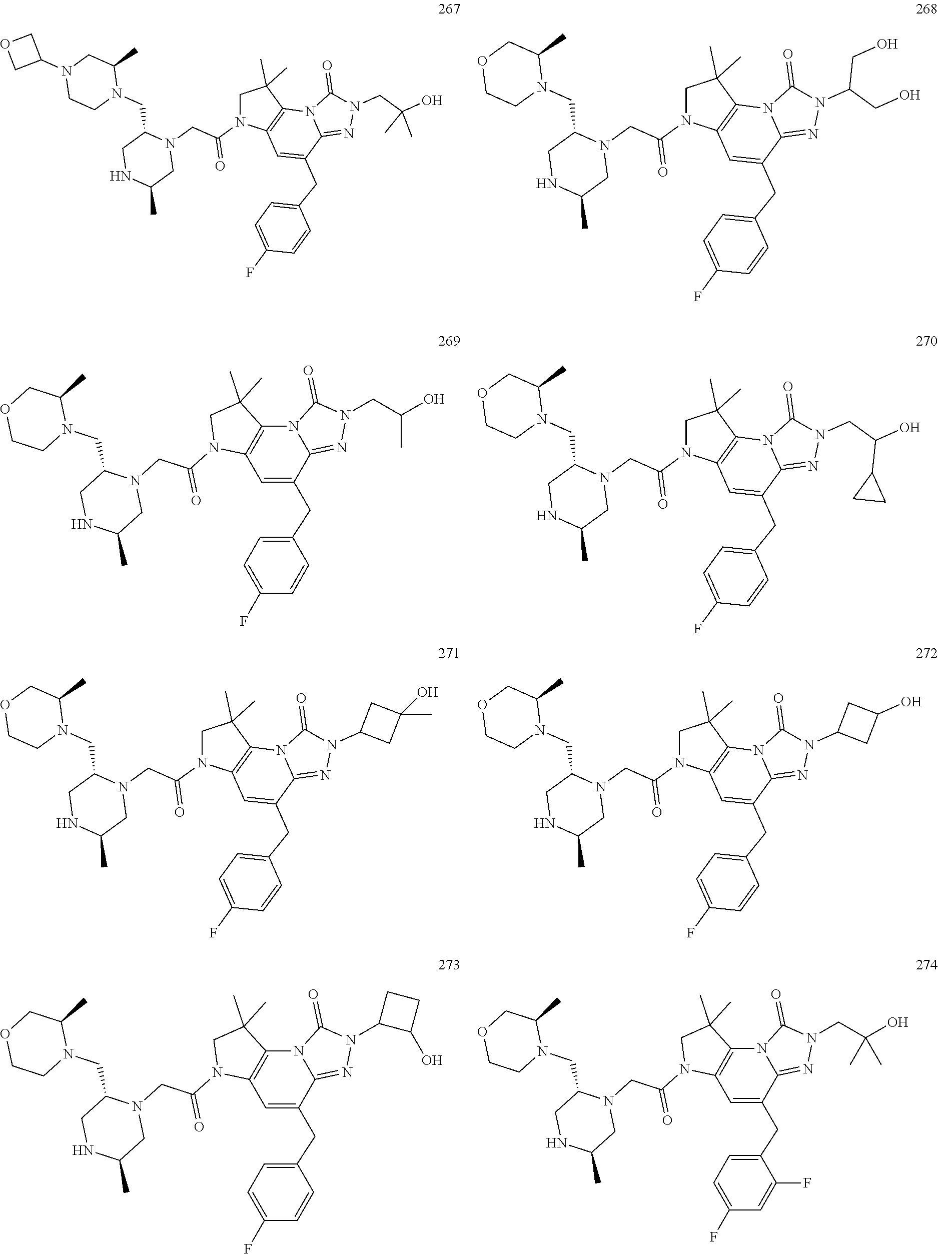

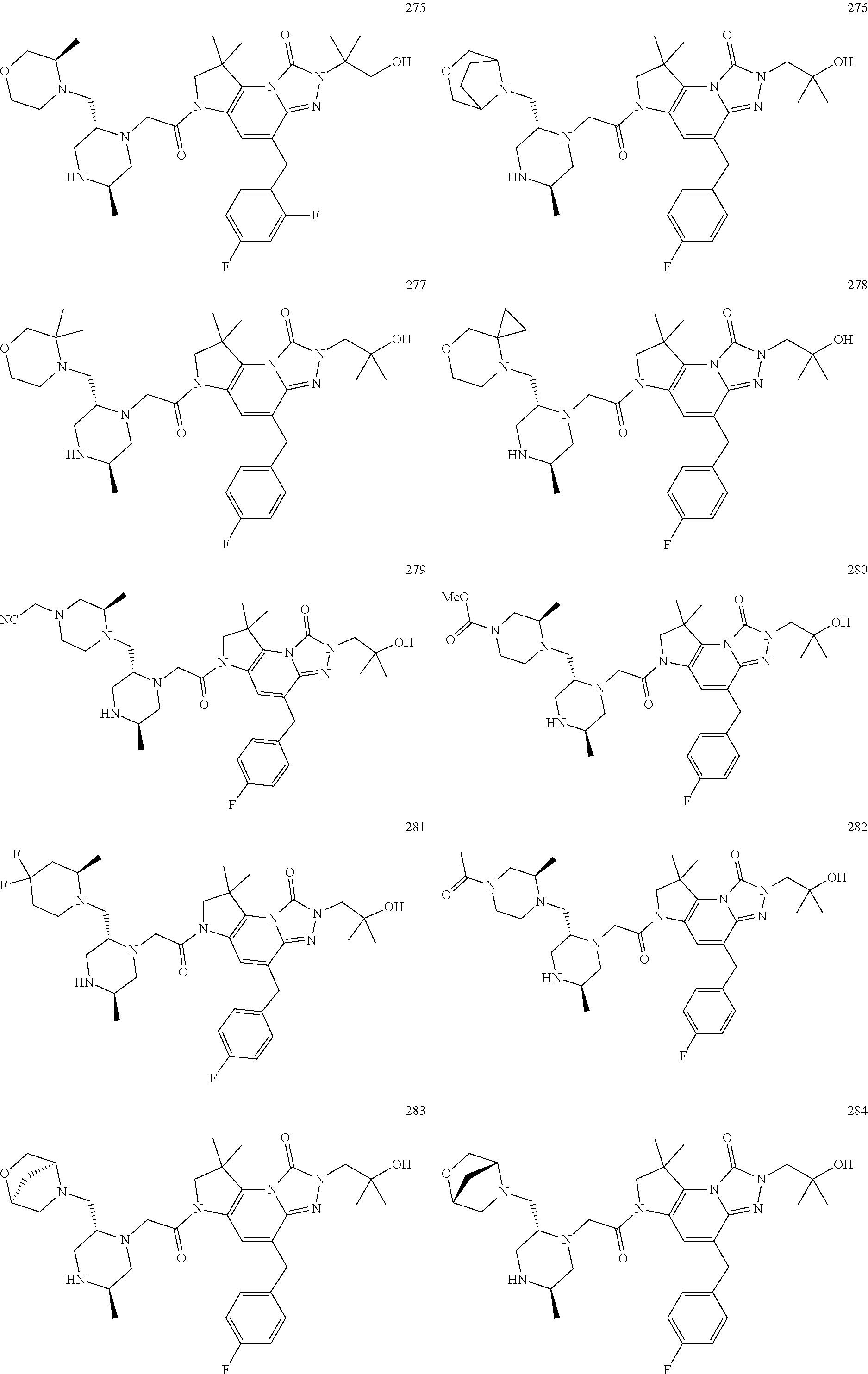

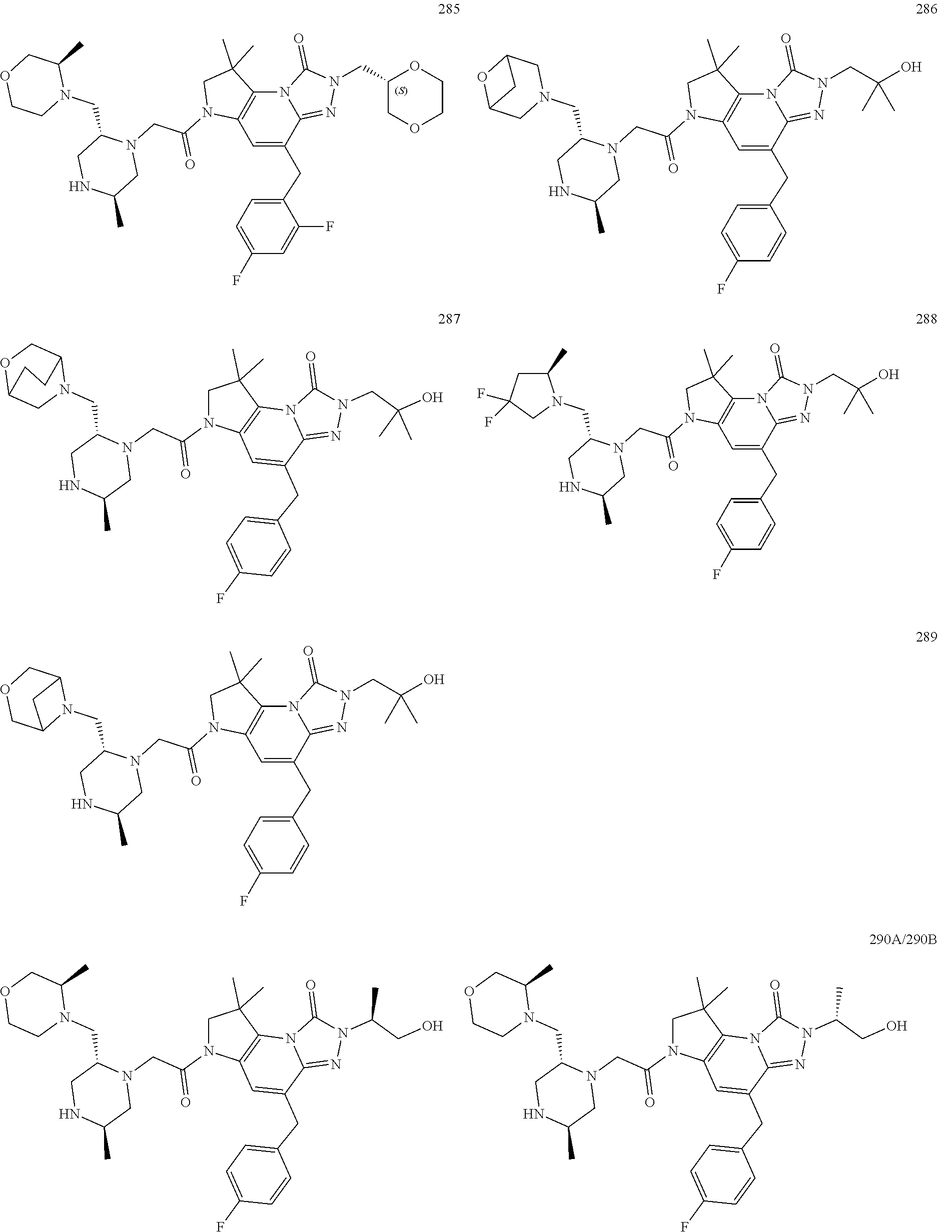

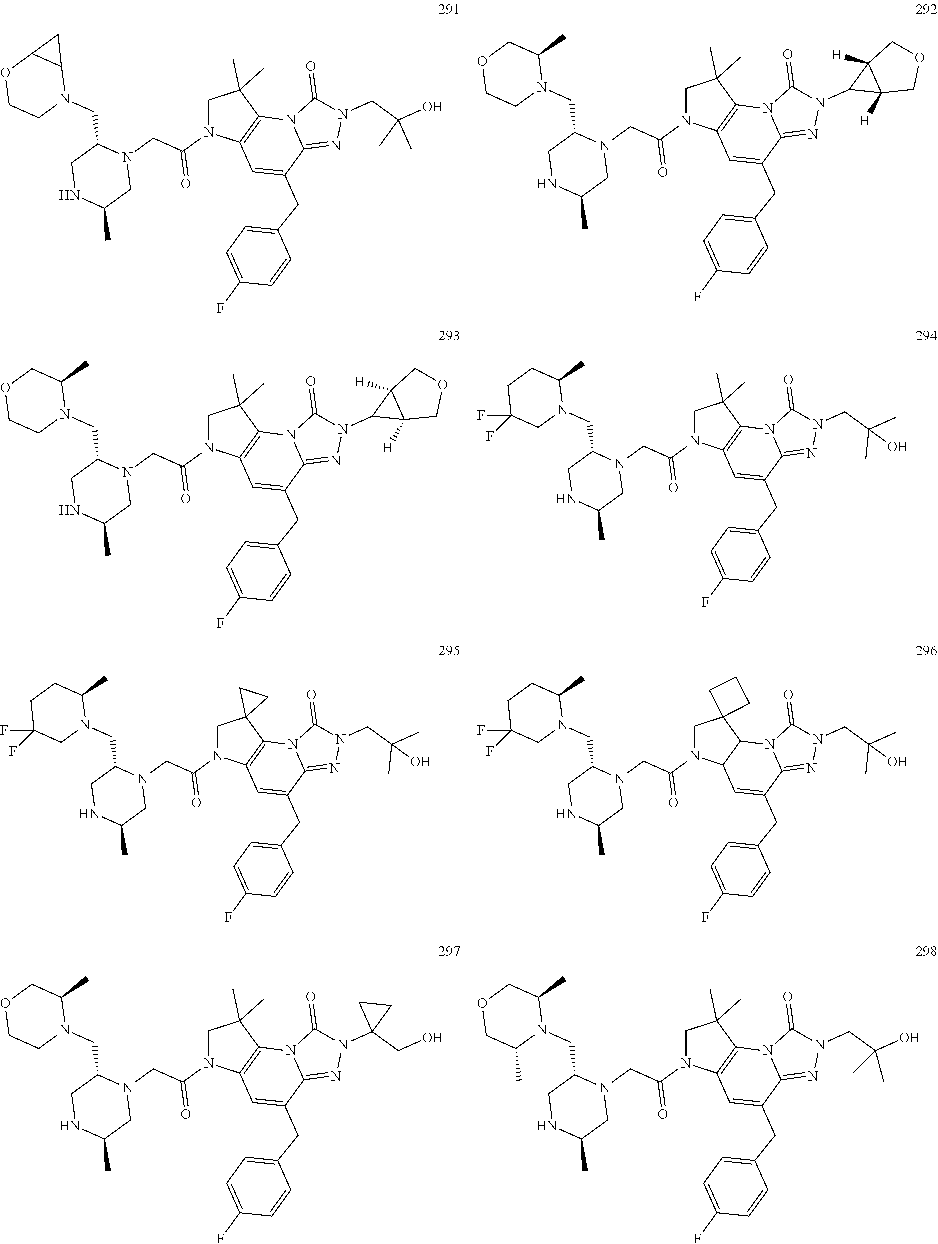

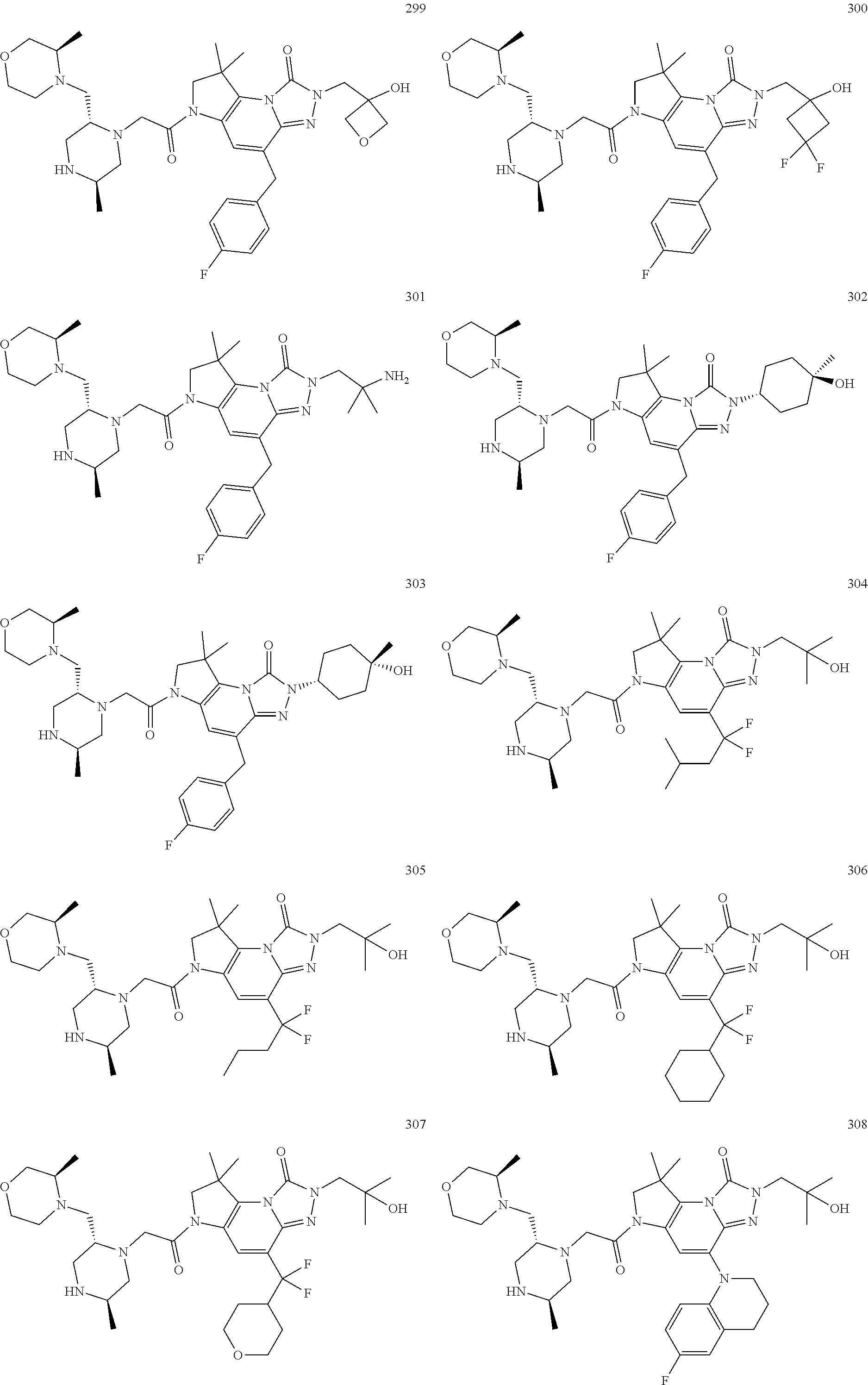

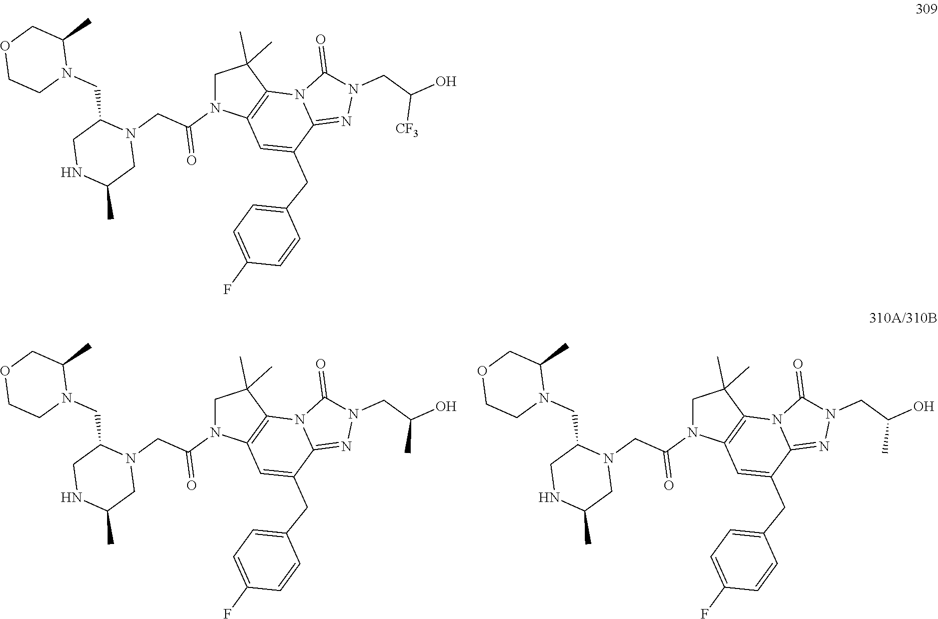

or a pharmaceutically acceptable salt or solvate thereof.

The present disclosure encompasses the preparation and use of salts of Compounds of the Disclosure. As used herein, the term “pharmaceutically acceptable salt” refers to salts or zwitterionic forms of Compounds of the Disclosure that are suitable for administration to a subject, e.g., a human. Salts of Compounds of the Disclosure can be prepared during the final isolation and purification of the compounds or separately by reacting the compound with a suitable acid. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of Compounds of the Disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

### III. Compositions

In one aspect, the present disclosure provides compositions comprising the compound of the disclosure, or a pharmaceutically acceptable salt or solvate thereof, and a pharmaceutically acceptable carrier.

Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins. In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

### IV. Methods and Uses

In one aspect, the present disclosure provides a method for inhibiting IAP protein activity in a cell, comprising contacting the cell in which inhibition of IAP protein activity is desired with an effective amount of a compound of the disclosure, or a pharmaceutically acceptable salt or solvate thereof; or a pharmaceutical composition of the disclosure.

In one embodiment, the contacting is in vitro. In one embodiment, the contacting is in vivo.

As used herein, the term “contacting” refers to the bringing together of indicated moieties in an in vitro system or an in vivo system. For example, “contacting” a IAP protein with a compound provided herein includes the administration of a compound provided herein to an individual or patient, such as a human, as well as, for example, introducing a compound provided herein into a sample containing a cellular or purified preparation containing the IAP protein.

In another aspect, the present disclosure provides a method for treating a disease or condition wherein inhibition of an IAP protein provides a benefit comprising administering a therapeutically effective amount of a compound of the disclosure, or a pharmaceutically acceptable salt or solvate thereof; or a pharmaceutical composition of the disclosure, to an individual in need thereof.

In some embodiments, the diseases and conditions that can be treated in accordance to the disclosure include, for example, cancers. A variety of cancers can be treated including, but not limited to: carcinomas, including bladder (including accelerated and metastic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, renal, and skin (including squamous cell carcinoma); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma, hematopoietic tumors of myeloid lineage, including acute and chronic myelogenous leukemias, myelodysplasia syndrome, myeloid leukemia, and promyelocytic leukemia; tumors of the central and peripheral nervous system, including astrocytoma, neuroblastoma, glioma, and schwannomas; tumors of mesenchymal origin, including fibrosarcoma, rhabdomyoscarcoma, and osteosarcoma; and other tumors, including melanoma, xenoderma pigmentosum, keratoactanthoma, seminoma, thyroid follicular cancer, teratocarcinoma, renal cell carcinoma (RCC), pancreatic cancer, myeloma, myeloid and lymphoblastic leukemia, neuroblastoma, and glioblastoma.

Additional forms of cancer treatable by the IAP protein inhibitors of the present disclosure include, for example, adult and pediatric oncology, growth of solid tumors/malignancies, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma), gastrointestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle-invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, and squamous cell cancer), thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

Another embodiment of the present disclosure is to induce apoptosis and potentiate the induction of apoptosis in response to apoptosis induction signals by use of an IAP protein inhibition of the present disclosure. The present IAP protein inhibitors also sensitize cells to inducers of apoptosis, including cells that are resistant to such inducers. The IAP protein inhibitors of the present disclosure can be used to induce apoptosis in any disorder that can be treated, ameliorated, or prevented by the induction of apoptosis. Thus, the present disclosure provides compositions and methods for targeting animals characterized as overexpressing an IAP protein. In some of the embodiments, the cells (e.g., cancer cells) show elevated expression levels of IAP proteins as compared to non-pathological samples (e.g., non-cancerous cells). In other embodiments, the cells operationally manifest elevated expression levels of IAP proteins by virtue of executing the apoptosis program and dying in response to a therapeutically effective amount of a compound of the present disclosure, said response occurring, at least in part, due to the dependence in such cells on IAP protein function for their survival.

In another embodiment, the present disclosure pertains to modulating an apoptosis-associated state which is associated with one or more apoptosis-modulating agents. Examples of apoptosis-modulating agents include, but are not limited to, Fas/CD95, TRAMP, TNF RI, DR1, DR2, DR3, DR4, DR5, DR6, FADD, RIP, TNFa, Fas ligand, TRAIL, antibodies to TRAIL-R1 or TRAIL-R2, Bcl-2, p53, BAX, BAD, Akt, CAD, PI3 kinase, PP1, and caspase proteins. Other agents involved in the initiation, decision, and degradation phase of apoptosis are also included. Examples of apoptosis-modulating agents include agents, the activity, presence, or change in concentration of which, can modulate apoptosis in a subject. Preferred apoptosis-modulating agents are inducers of apoptosis, such as TNF or a TNF-related ligand, particularly a TRAMP ligand, a Fas/CD95 ligand, a TNFR-1 ligand, or TRAIL.

These therapies can be used in a variety of settings for the treatment of various cancers. In a specific embodiment, the individual in need of treatment has previously undergone treatment for cancer. Such previous treatments include, but are not limited to, prior chemotherapy, radiotherapy, surgery, or immunotherapy, such as cancer vaccines.

In another embodiment, the present IAP protein inhibitors are used in methods of treating T and B cell mediated autoimmune diseases; inflammatory diseases; infections; hyperproliferative diseases; AIDS; degenerative conditions; vascular diseases; and the like. In some embodiments, infections suitable for treatment with the compositions and methods of the present invention include, but are not limited to, infections caused by viruses, bacteria, fungi, mycoplasma, prions, and the like.

The present compounds and methods also are useful in the treatment of autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjogren's syndrome, systemic lupus erythematosus, type 1 diabetes, ulcerative colitis, vitiligo, and the like.

Additional diseases and conditions, including cancers, that can be treated by administration of a present IAP protein inhibitor are disclosed in U.S. Pat. No. 7,960,372; incorporated herein by reference in its entirety.

A therapeutically effective amount of a Compound of the Disclosure required for use in therapy varies with the nature of the condition being treated, the length of time that activity is desired, and the age and the condition of the subject, and ultimately is determined by the attendant physician. Dosage amounts and intervals can be adjusted individually to provide plasma levels of the Compound of the Disclosure that are sufficient to maintain the desired therapeutic effects. The desired dose can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual subject, which can vary with the age, weight, and response of the particular subject.

In certain embodiments, the therapeutically effective amount of the compound is between about 0.01 to 100 mg/kg per day.

In another aspect, the present disclosure provides compound of the disclosure, or a pharmaceutically acceptable salt or solvate thereof, for use in inhibiting IAP protein activity in a cell.

In another aspect, the present disclosure provides compound of the disclosure, or a pharmaceutically acceptable salt or solvate thereof, for use in treating a disease or condition wherein inhibition of an IAP protein provides a benefit.

In another aspect, the present disclosure provides the use of the compound of the disclosure, or a pharmaceutically acceptable salt or solvate thereof, or the pharmaceutical composition of the disclosure, in the manufacture of a medicament for inhibiting IAP protein activity in a cell.

In another aspect, the present disclosure provides the use of the compound of the disclosure, or a pharmaceutically acceptable salt or solvate thereof, or the pharmaceutical composition of the disclosure, in the manufacture of a medicament for treating a disease or condition wherein inhibition of an IAP protein provides a benefit.

### V. Kits

In another embodiment, the present disclosure provides kits which comprise a Compound of the Disclosure (or a composition comprising a Compound of the Disclosure) packaged in a manner that facilitates their use to practice methods of the present disclosure. In one embodiment, the kit comprises Compound of the Disclosure (or a composition comprising a Compound of the Disclosure), and instructions for administering the compound, or a pharmaceutically acceptable salt or solvate thereof, to a subject for which the inhibition of an IAP protein provides a benefit, e.g., a subject having cancer. In one embodiment, the compound or composition is packaged in a unit dosage form. The kit further can include a device suitable for administering the composition according to the intended route of administration.

In some embodiments of the above various aspects, the IAP is preferably selected from cIAP1, cIAP2 and XIAP.

### ExampleS

In order to make the objects and technical solutions of the present disclosure clearer, the present disclosure will be further described below in conjunction with specific example. It should be understood that the examples are not intended to limit the scope of the invention. Further, specific experimental methods not mentioned in the following examples were carried out in accordance with a conventional experimental method.

### Intermediate

The following key intermediates were synthesized as literature reported.

| ID | Structure | IUPAC Name | Reference |
|---|---|---|---|
| I | 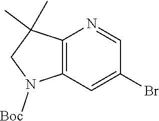 [SMILES: CC(C)(C)OC(=O)N1CC(C)(C)c2ncc(Br)cc21] | tert-butyl 6-bromo-3,3-dimethyl- 2,3-dihydro-1H-pyrrolo[3,2-b] pyridine-1-carboxylate | J. E. Day et al. Synlett 2015, 26 (18), 2570-2574. |
| II | 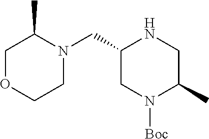 [SMILES: C[C@@H]1COCCN1C[C@H]1CN(C(=O)OC(C)(C)C)[C@H](C)CN1] | tert-butyl (2R,5S)-2-methyl-5- (((R)-3-methylmorpholino)methyl) piperazine-1-carboxylate | E. Tamanini et al. J. Med. Chem. 2017, 60, 4611-4625. |

### Representative Synthesis Examples

### Example 1: 1-(4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 1)

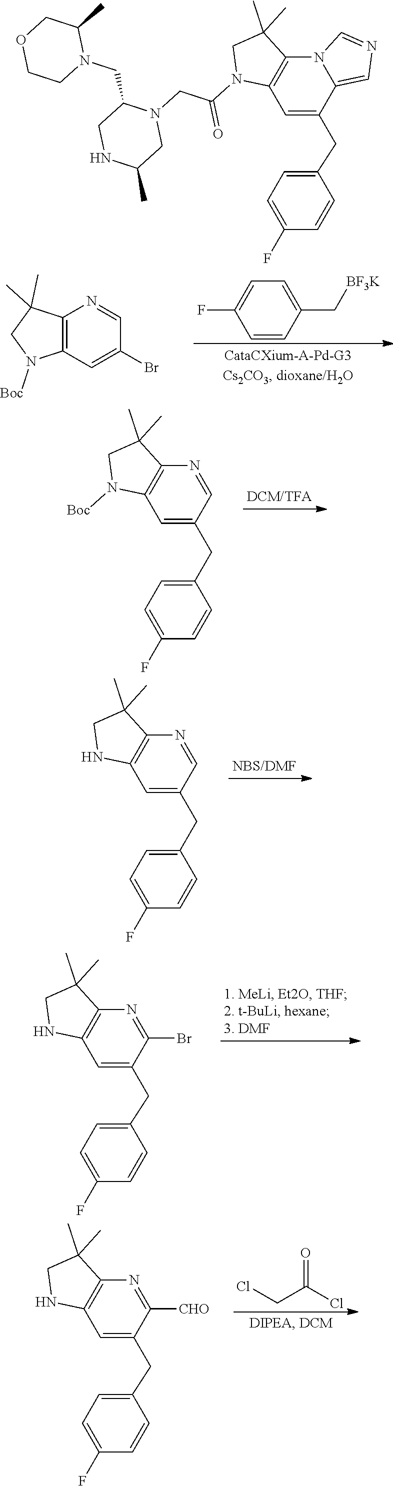

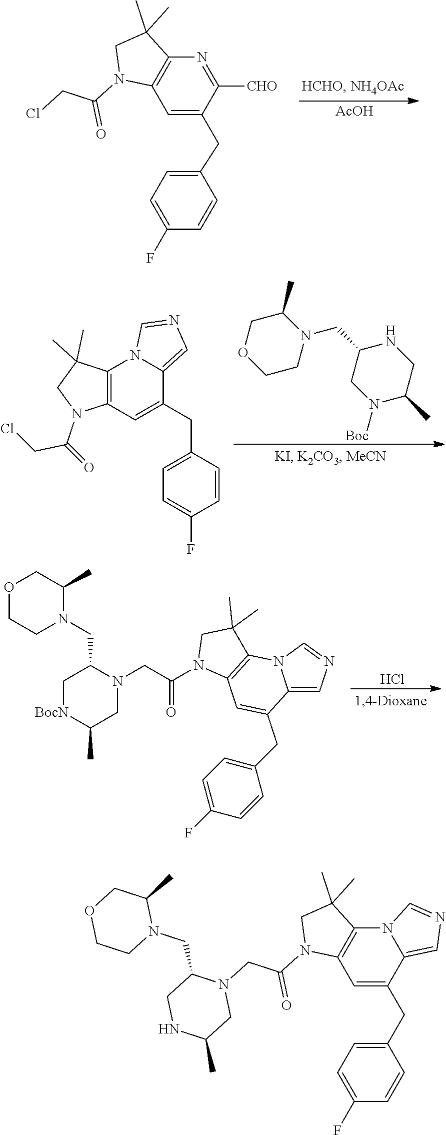

### Step 1: tert-butyl 6-(4-fluorobenzyl)-3,3-dimethyl-2,3-dihydro-1H-pyrrolo[3,2-b]pyridine-1-carboxylate

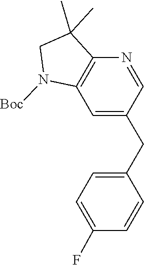 [SMILES: CC(C)(C)OC(=O)N1CC(C)(C)c2ncc(Cc3ccc(F)cc3)cc21]

In a flame-dried 100 mL round-bottomed flask, tert-butyl 6-bromo-3,3-dimethyl-2,3-dihydro-1H-pyrrolo[3,2-b]pyridine-1-carboxylate I (3 g, 9.17 mmol), potassium trifluoro(4-fluorobenzyl)borate (3.96 g, 18.34 mmol) and Cs₂CO₃ (8.96 g, 27.5 mmol) was dissolved in 1,4-Dioxane (50 ml)/Water (5 ml) under nitro gen at r.t. to give a color solution. CataCXium-A-Pd-G3 (0.668 g, 0.92 mmol) was added to the reaction mixture at r.t. The reaction mixture was filled N₂ three times and stirred at 95° C. for 10 h, then saturated NaHCO₃ (100 ml) was added to the reaction mixture followed by extraction with ethyl acetate. The combined organic layers were dried over Na₂SO₄, filtered and concentrated. The crude product was added to a silica gel column and was eluted with ethyl acetate/hexane=1:10 to give tert-butyl 6-(4-fluorobenzyl)-3,3-dimethyl-2,3-dihydro-1H-pyrrolo[3,2-b]pyridine-1-carboxylate (2.9 g, 89%) as a white solid. MS (ESI) m/z 357.2 [M+H]⁺.

### Step 2: 6-(4-fluorobenzyl)-3,3-dimethyl-2,3-dihydro-1H-pyrrolo[3,2-b]pyridine

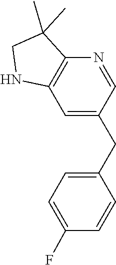 [SMILES: CC1(C)CNc2cc(Cc3ccc(F)cc3)cnc21]

To a solution of tert-butyl 6-(4-fluorobenzyl)-3,3-dimethyl-2,3-dihydro-1H-pyrrolo[3,2-b]pyridine-1-carboxylate (1 g, 2.81 mmol) in DCM (10 ml) was added TFA (10 ml). The mixture was stirred at r.t. for 1 h. The mixture was concentrated and diluted with EtOAc, washed by Sat·NaHCO₃·aq and brine. The organic layer was dried over by Na₂SO₄, filtered and concentrated. The crude pro duct was added to a silica gel column and eluted with DCM/MeOH (DCM:MeOH, 10:1) to afford the title compound (660 mg, 93%) as a white solid. MS (ESI) m/z 257.2 [M+H]⁺.

### Step 3: 5-bromo-6-(4-fluorobenzyl)-3,3-dimethyl-2,3-dihydro-1H-pyrrolo[3,2-b]pyridine

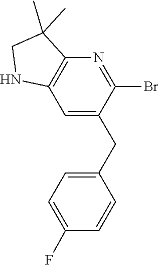 [SMILES: CC1(C)CNc2cc(Cc3ccc(F)cc3)c(Br)nc21]

In a flame-dried 50 mL round-bottomed flask, 6-(4-fluorobenzyl)-3,3-dimeth yl-2,3-dihydro-1H-pyrrolo[3,2-b]pyridine (0.6 g, 2.341 mmol) was dissolved in DMF (20 ml) under nitrogen to give a color solution. NBS (0.42 g, 2.341 mmol) was added dropwise to the reaction mixture at −5-0° C. The reaction mixture was stirred at 0° C. for 2 h. saturated NaHCO₃ was added to the reaction mixture followed by extraction with ethyl acetate. The combined organic layers were dried over Na₂SO₄, filtered and concentrated. The crude product was added to a silica gel column and was eluted with ethyl acetate/hexane (ethyl acetate:hexane=1:5) to give 5-bromo-6-(4-fluorobenzyl)-3,3-dimethyl-2,3-dihydro-1H-pyrrolo[3,2-b]pyridine (0.6 g, 76%) as a white solid. MS (ESI) m/z 335.1 [M+H]⁺.

### Step 4: 6-(4-fluorobenzyl)-3,3-dimethyl-2,3-dihydro-1H-pyrrolo[3,2-b]pyridine-5-carbaldehyde

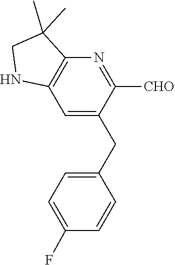 [SMILES: CC1(C)CNc2cc(Cc3ccc(F)cc3)c(C=O)nc21]

To 5-bromo-6-(4-fluorobenzyl)-3,3-dimethyl-2,3-dihydro-1H-pyrrolo[3,2-b]pyri dine (2.5 g, 7.46 mmol) in THF (40 mL), cooled to −78° C., was added meth yllithium (1.6 M in Et₂O; 6.25 mL, 10 mmol) over 15 min tert-butyllithium (1.7 M in hexane; 10.5 mL, 17.9 mmol) was then added over 30 min. After 15 min, DMF (3 mL) was added, and the mixture was stirred at −78° C. for a further 50 min. Saturated aqueous NH₄Cl (45 mL) was added, and the mixture was stirred for 10 min at RT. The organic layer was isolated, and the aqueous layer was extracted with ethyl acetate. The combined organic layer were washed with brine, dried over Na₂SO₄ and evaporated to give a crude residue, which was purified by silica gel flash column chromatography eluted with ethyl acetate/hexane to give 6-(4-fluorobenzyl)-3,3-dimethyl-2,3-dihydro-1H-pyrrolo[3,2-b]pyridine-5-carbaldehyde as a white solid (1.3 g, 61% yield). MS (ESI) m/z 285.3 [M+H]⁺.

### Step 5: 1-(2-chloroacetyl)-6-(4-fluorobenzyl)-3,3-dimethyl-2,3-dihydro-1H-pyrrolo[3,2-b]pyridine-5-carbaldehyde

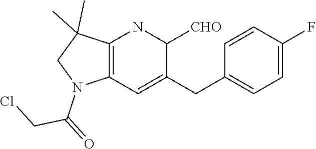 [SMILES: CC1(C)CN(C(=O)CCl)C2=C1NC(C=O)C(Cc1ccc(F)cc1)=C2]

In a flame-dried 50 mL round-bottomed flask, 6-(4-fluorobenzyl)-3,3-dimeth yl-2,3-dihydro-1H-pyrrolo[3,2-b]pyridine-5-carbaldehyde (1 g, 3.52 mmol, 1.0 eq) and DIPEA (0.909 g, 7.03 mmol, 2.0 eq) were dissolved in CH₂Cl₂ (50 mL) under nitrogen to give a solution. 2-chloroacetyl chloride (0.477 g, 4.22 mmol, 1.2 eq) was added to the reaction mixture at 0° C. The reaction mixture was stirred at 22° C. for 1 h, then saturated NaHCO₃ (10 mL) was added to the reaction mixture followed by extraction with dichloromethane (15 mL×3). The combined organic layers were dried over Na₂SO₄, filtered and concentrated. The crude product was added to a silica gel column and was eluted with ethyl acetate/hexane=1:2 to give 1-(2-chloroacetyl)-6-(4-fluorobenzyl)-3,3-dimethyl-2,3-dihydro-1H-pyrrolo[3,2-b]pyridine-5-carbaldehyde (1 g, 79%) as an off-white solid. MS (ESI) m/z 361.5 [M+H]⁺.

### Step 6: 2-chloro-1-(4-(4-fluorobenzyl)-8,8-dimethyl-7,8-dihydro-6H-imidazo[1,5-a]pyrrolo[2,3-e]pyridin-6-yl)ethan-1-one

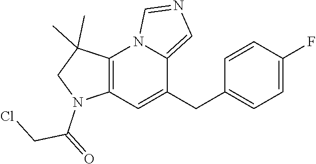 [SMILES: CC1(C)CN(C(=O)CCl)c2cc(Cc3ccc(F)cc3)c3cncn3c21]

In a flame-dried 100 mL round-bottomed flask, 1-(2-chloroacetyl)-6-(4-fluorobenzyl)-3,3-dimethyl-2,3-dihydro-1H-pyrrolo[3,2-b]pyridine-5-carbaldehyde (40 mg, 0.111 mmol, 1.0 eq) and ammonium acetate (51.3 mg, 0.665 mmol, 6.0 eq) were dissolved in acetic acid (1.0 mL) under nitrogen to give a solution. Formaldehyde (16.64 mg, 0.554 mmol, 5.0 eq) was added to the reaction mixture dropwise at 22 T. The reaction mixture was stirred at 22° C. for 16 h, then saturated NaHCO₃ (10 mL) was added to the reaction mixture followed by ex traction with ethyl acetate (15 mL×3). The combined organic layers were dried over Na₂SO₄, filtered and concentrated. The crude product was added to a silica gel column and was eluted with ethyl acetate/hexane=1:2 to give 2-chloro-1-(4-(4-fluorobenzyl)-8,8-dimethyl-7,8-dihydro-6H-imidazo[1,5-a]pyrrolo[2,3-e]pyridin-6-yl)ethan-1-one (30 mg, 72.8%) as an off-white solid. MS (ESI) m/z 387.3 [M+H]⁺.

Step 7: tert-butyl (2R,5S)-4-(2-(4-(4-fluorobenzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

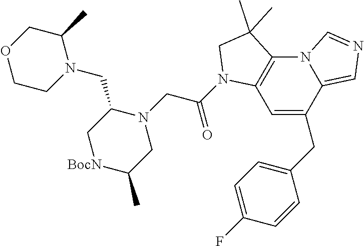 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1cncn21]

In a flame-dried 50 mL round-bottomed flask, 2-chloro-1-(4-(4-fluorobenzyl)-8,8-dimethyl-7,8-dihydro-6H-imidazo[1,5-a]pyrrolo[2,3-e]pyridin-6-yl)ethan-1-one (30 mg, 0.081 mmol, 1.0 eq), potassium iodide (20.09 mg, 0.121 mmol, 1.5 eq) and K₂CO₃ (33.5 mg, 0.242 mmol, 3.0 eq) were dissolved in Acetonitrile (5 mL) under nitrogen to give a solution. tert-butyl (2R,5S)-2-methyl-5-(((R)-3-methylmorpholino)methyl)piperazine-1-carboxylate II (27.8 mg, 0.089 mmol, 1.1 eq) was added to the reaction mixture at 0° C. The reaction mixture was stir red at r.t. for 3 h. saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:20 to give tert-butyl (2R,5S)-4-(2-(4-(4-fluorobenzyl)-8,8-dimethyl-7,8-di hydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (35 mg, 66.9%) as an off-white solid. MS (ESI) m/z 649.9 [M+H]⁺.

### Step 8: 1-(4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

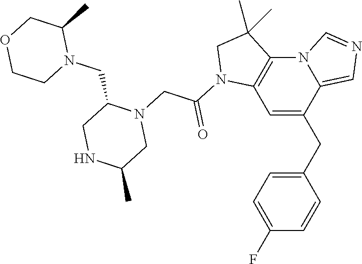 [SMILES: C[C@@H]1CN(CC(=O)N2CC(C)(C)c3c2cc(Cc2ccc(F)cc2)c2cncn32)[C@@H](CN2CCOC[C@H]2C)CN1]

In a flame-dried 50 mL round-bottomed flask, tert-butyl (2R,5S)-4-(2-(4-(4-fluorobenzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (45 mg, 0.069 mmol, 1.0 eq) was dissolved in 1,4-Dioxane (1 mL) under nitrogen to give a solution. HCl (1 mL, 4M in 1,4-Dioxane) was added to the reaction mixture at 22° C. The reaction mixture was stirred at 22° C. for 2 h. saturated NaHCO₃ (10 mL) was added to the reaction mixture followed by extraction with dichloromethane (10 mL×3). The combined organic layers were dried over Na₂SO₄, filtered and concentrated. The crude product was added to a silica gel column and was eluted with methanol/dichloromethane 1:20 to give 1-(4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (7.3 mg, 19%) as an off-white solid. ¹H NMR (400 MHz, DMSO-d⁶) δ (ppm) 8.53 (s, 1H), 7.73 (s, 1H), 7.57 (s, 1H), 7.36 (dd, J=8.5, 5.7 Hz, 2H), 7.12 (t, J=8.9 Hz, 2H), 4.10 (s, 2H), 4.07 (d, J=10.6 Hz, 1H), 3.86 (d, J=10.6 Hz, 1H), 3.77 (d, J=16.5 Hz, 1H), 3.61 (d, J=16.4 Hz, 1H), 3.50 (d, J=10.9 Hz, 2H), 3.21 (t, J=9.4 Hz, 1H), 3.00-2.89 (m, 2H), 2.85 (t, J=10.8 Hz, 2H), 2.71 (d, J=9.3 Hz, 1H), 2.61 (d, J=8.9 Hz, 2H), 2.45 (t, J=11.1 Hz, 1H), 2.28 (t, J=11.1 Hz, 1H), 2.16 (brs, 1H), 1.93 (t, J=9.4 Hz, 1H), 1.75 (d, J=11.5 Hz, 1H), 1.56 (s, 3H), 1.52 (s, 3H), 0.91 (d, J=6.2 Hz, 3H), 0.88 (d, J=6.1 Hz, 3H). MS (ESI) m/z 549.7 [M+H]⁺.

### Example 2: 1-(4-(4-fluorobenzyl)-1,8,8-trimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 2)

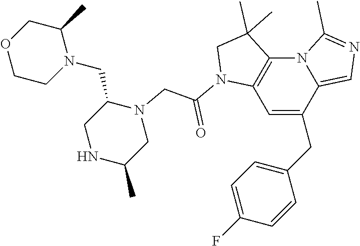 [SMILES: Cc1ncc2c(Cc3ccc(F)cc3)cc3c(n12)C(C)(C)CN3C(=O)CN1C[C@@H](C)NC[C@@H]1CN1CCOC[C@H]1C]

### Step 1: 2-chloro-1-(4-(4-fluorobenzyl)-1,8,8-trimethyl-7,8-dihydro-6H-imidazo[1,5-a]pyrrolo[2,3-e]pyridin-6-yl)ethan-1-one

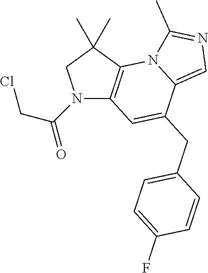 [SMILES: Cc1ncc2c(Cc3ccc(F)cc3)cc3c(n12)C(C)(C)CN3C(=O)CCl]

In a flame-dried 100 mL round-bottomed flask, 1-(2-chloroacetyl)-6-(4-fluoro benzyl)-3,3-dimethyl-2,3-dihydro-1H-pyrrolo[3,2-b]pyridine-5-carbaldehyde (50 mg, 0.139 mmol, 1.0 eq) and ammonium acetate (64.1 mg, 0.831 mmol, 6.0 eq) were dissolved in acetic acid (1.0 mL) under nitrogen to give a solution. Acetaldehyde (30.5 mg, 0.693 mmol, 5.0 eq) was added to the reaction mixture dropwise at 22° C. The reaction mixture was stirred at 22° C. for 16 h, then saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:40 to give 2-chloro-1-(4-(4-fluorobenzyl)-1,8,8-trimethyl-7,8-dihydro-6H-imidazo[1,5-a]pyrrolo[2,3-e]pyridin-6-yl)ethan-1-one (32 mg, 59.8%) as an off-white solid. MS (ESI) m/z 386.4 [M+H]⁺.

Step 2: tert-butyl (2R,5S)-4-(2-(4-(4-fluorobenzyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

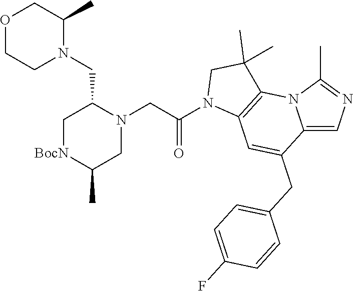 [SMILES: Cc1ncc2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C]

In a flame-dried 50 mL round-bottomed flask, 2-chloro-1-(4-(4-fluorobenzyl)-1,8,8-trimethyl-7,8-dihydro-6H-imidazo[1,5-a]pyrrolo[2,3-e]pyridin-6-yl)ethan-1-one (32 mg, 0.083 mmol, 1.0 eq), potassium iodide (20.65 mg, 0.124 mmol, 1.5 eq) and K₂CO₃ (34.4 mg, 0.249 mmol, 3.0 eq) were dissolved in acetonitrile (5 mL) under nitrogen to give a solution. tert-butyl (2R,5S)-2-methyl-5-(((R)-3-methylmorpholino)methyl)piperazine-1-carboxylate II (28.6 mg, 0.091 mmol, 1.1 eq) was added to the reaction mixture at 0° C. The reaction mixture was stir red at r.t. for 3 h. Saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:20 to give tert-butyl (2R,5S)-4-(2-(4-(4-fluorobenzyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (40 mg, 72.8%) as an off-white solid. MS (ESI) m/z 663.7 [M+H]⁺.

Step 3: 1-(4-(4-fluorobenzyl)-1,8,8-trimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

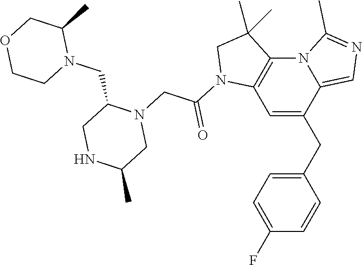 [SMILES: Cc1ncc2c(Cc3ccc(F)cc3)cc3c(n12)C(C)(C)CN3C(=O)CN1C[C@@H](C)NC[C@@H]1CN1CCOC[C@H]1C]

In a flame-dried 50 mL round-bottomed flask, tert-butyl (2R,5S)-4-(2-(4-(4-fluorobenzyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (40 mg, 0.060 mmol, 1.0 eq) was dissolved in 1,4-Dioxane (1.0 mL) under nitrogen to give a solution. HCl (1 mL, 4M in 1,4-Dioxane) was added to the reaction mixture at 22° C. The reaction mixture was stirred at 22° C. for 2 h. Saturated NaHCO₃ (10 mL) was added to the reaction mixture followed by extraction with dichloromethane (10 mL×3). The combined organic layers were dried over Na₂SO₄, filtered and concentrated. The crude product was added to a silica gel column and was eluted with methanol/dichloromethane=1:20 to give 1-(4-(4-fluorobenzyl)-1,8,8-trimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (15 mg, 44.2%) as an off-white solid. ¹H NMR (400 MHz, DMSO-d⁶) δ (ppm) 7.70 (s, 1H), 7.48 (s, 1H), 7.34 (dd, J=8.5, 5.6 Hz, 2H), 7.11 (t, J=8.9 Hz, 2H), 4.05 (d, J=9.9 Hz, 3H), 3.87-3.76 (m, 2H), 3.62-3.45 (m, 3H), 3.23 (t, J=9.8 Hz, 1H), 2.99-2.77 (m, 7H), 2.72 (d, J=10.8 Hz, 1H), 2.61 (d, J=8.9 Hz, 2H), 2.41 (t, J=11.0 Hz, 1H), 2.28 (t, J=10.7 Hz, 1H), 2.16 (brs, 1H), 1.94 (t, J=9.4 Hz, 1H), 1.75 (d, J=11.3 Hz, 1H), 1.54 (d, J=16.4 Hz, 6H), 0.91 (d, J=6.2 Hz, 3H), 0.88 (d, J=6.1 Hz, 3H). MS (ESI) m/z 563.5 [M+H]⁺.

Example 3: 1-(1-cyclopropyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 3)

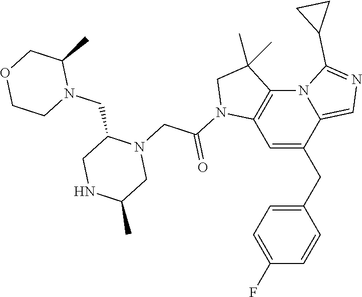 [SMILES: C[C@@H]1CN(CC(=O)N2CC(C)(C)c3c2cc(Cc2ccc(F)cc2)c2cnc(C4CC4)n32)[C@@H](CN2CCOC[C@H]2C)CN1]

Step 1: 2-chloro-1-(1-cyclopropyl-4-(4-fluorobenzyl)-8,8-dimethyl-7,8-dihydro-6H-imidazo[1,5-a]pyrrolo[2,3-e]pyridin-6-yl)ethan-1-one

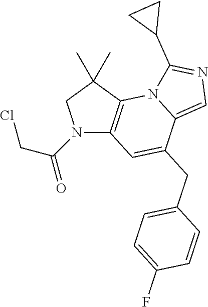 [SMILES: CC1(C)CN(C(=O)CCl)c2cc(Cc3ccc(F)cc3)c3cnc(C4CC4)n3c21]

In a flame-dried 100 mL round-bottomed flask, 1-(2-chloroacetyl)-6-(4-fluorobenzyl)-3,3-dimethyl-2,3-dihydro-1H-pyrrolo[3,2-b]pyridine-5-carbaldehyde (50 mg, 0.139 mmol, 1.0 eq) and ammonium acetate (64.1 mg, 0.831 mmol, 6.0 eq) were dissolved in acetic acid (1 mL) under nitrogen to give a solution. Cyclopropanecarbaldehyde (48.6 mg, 0.693 mmol, 5.0 eq) was added to the reaction mixture dropwise at 22° C. The reaction mixture was stirred at 22° C. for 16 h, then saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:40 to give 2-chloro-1-(1-cyclopropyl-4-(4-fluorobenzyl)-8,8-dimethyl-7,8-dihydro-6H-imidazo[1,5-a]pyrrolo[2,3-e]pyridin-6-yl)ethan-1-one (40 mg, 70%) as an off-white solid. MS (ESI) m/z 413.4 [M+H]⁺.

Step 2: tert-butyl (2R,5S)-4-(2-(1-cyclopropyl-4-(4-fluorobenzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

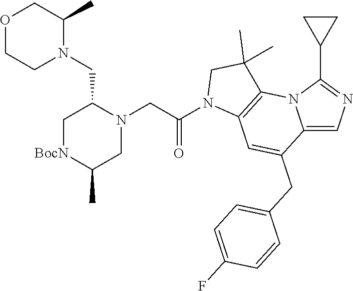 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1cnc(C3CC3)n21]

In a flame-dried 50 mL round-bottomed flask, 2-chloro-1-(1-cyclopropyl-4-(4-fluorobenzyl)-8,8-dimethyl-7,8-dihydro-6H-imidazo[1,5-a]pyrrolo[2,3-e]pyridin-6-yl)ethan-1-one (35 mg, 0.085 mmol, 1.0 eq), potassium iodide (28.2 mg, 0.170 mmol, 2.0 eq) and K₂CO₃ (35.2 mg, 0.255 mmol, 3.0 eq) were dissolved in acetonitrile (5.0 mL) under nitrogen to give a solution. Tert-butyl (2R,5S)-2-m ethyl-5-(((R)-3-methylmorpholino)methyl)piperazine-1-carboxylate 1 (27 mg, 0.093 mmol, 1.1 eq) was added to the reaction mixture at 0° C. The reaction mixture was stirred at r.t. for 3 h. Saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:20 to give tert-butyl (2R,5S)-4-(2-(1-cyclopropyl-4-(4-fluoro benzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (45 mg, 77%) as an off-white solid. MS (ESI) m/z 690.1 [M+H]⁺.

Step 3: 1-(1-cyclopropyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

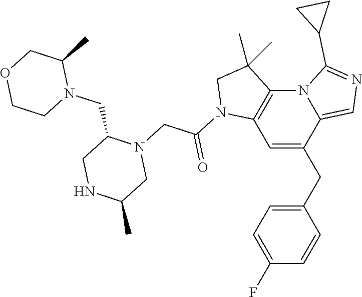 [SMILES: C[C@@H]1CN(CC(=O)N2CC(C)(C)c3c2cc(Cc2ccc(F)cc2)c2cnc(C4CC4)n32)[C@@H](CN2CCOC[C@H]2C)CN1]

In a flame-dried 50 mL round-bottomed flask, tert-butyl (2R,5S)-4-(2-(1-cyclopropyl-4-(4-fluorobenzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (45 mg, 0.065 mmol, 1.0 eq) was dissolved in 1,4-Dioxane (1 mL) under nitrogen to give a solution. HCl (1 mL, 4M in 1,4-Dioxane) was added to the reaction mixture at 22° C. The reaction mixture was stirred at 22° C. for 2 h. Saturated NaHCO₃ (10 mL) was added to the reaction mixture followed by extraction with dichloromethane (10 mL×3). The combined organic layers were dried over Na₂SO₄, filtered and concentrated. The crude product was added to a silica gel column and was eluted with methanol/dichloromethane=1:20 to give 1-(1-cyclopropyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (15 mg, 39%) as an off-white solid. ¹H NMR (400 MHz, DMSO-d⁶) δ (ppm) 7.67 (s, 1H), 7.44 (s, 1H), 7.34 (dd, J=8.5, 5.6 Hz, 2H), 7.11 (t, J=8.9 Hz, 2H), 4.08 (d, J=10.8 Hz, 1H), 4.02 (s, 2H), 3.88-3.76 (m, 2H), 3.59 (d, J=16.6 Hz, 1H), 3.51 (d, J=11.1 Hz, 2H), 3.22 (t, J=10.1 Hz, 1H), 2.99-2.89 (m, 2H), 2.87 (d, J=12.8 Hz, 1H), 2.82 (d, J=11.8 Hz, 1H), 2.72 (d, J=10.3 Hz, 1H), 2.61 (d, J=8.9 Hz, 2H), 2.42 (t, J=10.7 Hz, 1H), 2.37 (dd, J=9.4, 4.4 Hz, 1H), 2.28 (t, J=10.8 Hz, 1H), 2.15 (brs, 1H), 1.93 (t, J=9.6 Hz, 1H), 1.75 (d, J=11.9 Hz, 1H), 1.63 (d, J=18.0 Hz, 6H), 1.14 (t, J=11.1 Hz, 1H), 1.10-1.01 (m, 3H), 0.91 (d, J=6.1 Hz, 3H), 0.88 (d, J=6.1 Hz, 3H). MS (ESI) m/z 589.6 [M+H]⁺.

Example 4: 1-(1-ethyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 4)

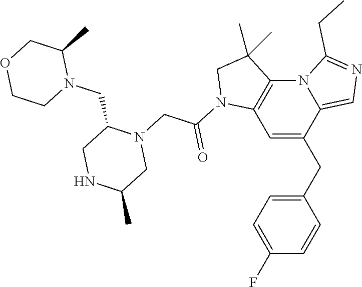 [SMILES: CCc1ncc2c(Cc3ccc(F)cc3)cc3c(n12)C(C)(C)CN3C(=O)CN1C[C@@H](C)NC[C@@H]1CN1CCOC[C@H]1C]

Step 1: 2-chloro-1-(1-ethyl-4-(4-fluorobenzyl)-8,8-dimethyl-7,8-dihydro-6H-imidazo[1,5-a]pyrrolo[2,3-e]pyridin-6-yl)ethan-1-one

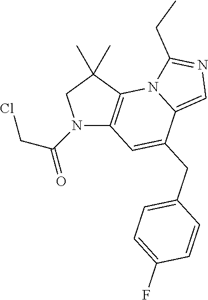 [SMILES: CCc1ncc2c(Cc3ccc(F)cc3)cc3c(n12)C(C)(C)CN3C(=O)CCl]

In a flame-dried 100 mL round-bottomed flask, 1-(2-chloroacetyl)-6-(4-fluorobenzyl)-3,3-dimethyl-2,3-dihydro-1H-pyrrolo[3,2-b]pyridine-5-carbaldehyde (55 mg, 0.152 mmol, 1.0 eq) and ammonium acetate (70.5 mg, 0.915 mmol, 6.0 eq) were dissolved in acetic acid (1 mL) under nitrogen to give a solution. propionaldehyde (44.3 mg, 0.762 mmol, 5.0 eq) was added to the reaction mixture dropwise at 22° C. The reaction mixture was stirred at 22° C. for 16 h, then saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:40 to give 2-chloro-1-(1-ethyl-4-(4-fluorobenzyl)-8,8-dimethyl-7,8-dihydro-6H-imidazo[1,5-a]pyrrolo[2,3-e]pyridin-6-yl)ethan-1-one (45 mg, 73.8%) as an off-white solid. MS (ESI) m/z 401.4 [M+H]⁺.

Step 2: tert-butyl (2R,5S)-4-(2-(1-ethyl-4-(4-fluorobenzyl)-8,8-dimethyl-7,8-di hydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

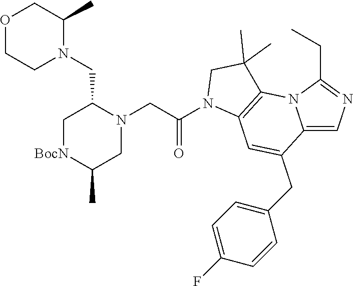 [SMILES: CCc1ncc2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C]

In a flame-dried 50 mL round-bottomed flask, 2-chloro-1-(1-ethyl-4-(4-fluorobenzyl)-8,8-dimethyl-7,8-dihydro-6H-imidazo[1,5-a]pyrrolo[2,3-e]pyridin-6-yl)ethan-1-one (45 mg, 0.113 mmol, 1.0 eq), potassium iodide (37.4 mg, 0.225 mmol, 2.0 eq) and K₂CO₃ (46.7 mg, 0.338 mmol, 3.0 eq) were dissolved in acetonitrile (5 mL) under nitrogen to give a solution. tert-butyl (2R,5S)-2-methyl-5-(((R)-3-methylmorpholino)methyl)piperazine-1-carboxylate II (36 mg, 0.12 mmol, 1.1 eq) was added to the reaction mixture at 0° C. The reaction mixture was stir red at r.t. for 3 h. Saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:20 to give tert-butyl (2R,5S)-4-(2-(1-ethyl-4-(4-fluorobenzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (50 mg, 65.6%) as an off-white solid. MS (ESI) m/z 677.6 [M+H]⁺.

Step 3: 1-(1-ethyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

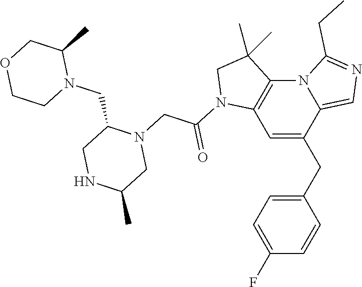 [SMILES: CCc1ncc2c(Cc3ccc(F)cc3)cc3c(n12)C(C)(C)CN3C(=O)CN1C[C@@H](C)NC[C@@H]1CN1CCOC[C@H]1C]

In a flame-dried 50 mL round-bottomed flask, tert-butyl (2R,5S)-4-(2-(1-eth yl-4-(4-fluorobenzyl)-8,8-dimethyl-7,8-dihydro-6H-imidazo[1,5-a]pyrrolo[2,3-e]pyrid in-6-yl)-2-oxoethyl)-2-methyl-5-(((R)-3-methylmorpholino)methyl)piperazine-1-carboxylate (45 mg, 0.066 mmol, 1.0 eq) was dissolved in 1,4-Dioxane (1 mL) under nitrogen to give a solution. HCl (1 mL, 4M in 1,4-Dioxane) was added to the reaction mixture at 22° C. The reaction mixture was stirred at 22° C. for 2 h. saturated NaHCO₃ (10 mL) was added to the reaction mixture followed by extraction with dichloromethane (10 mL×3). The combined organic layers were dried over Na₂SO₄, filtered and concentrated. The crude product was added to a silica gel column and was eluted with methanol/dichloromethane=1:20 to give 1-(1-ethyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)meth yl)piperazin-1-yl)ethan-1-one (8.5 mg, 22.17%) as an off-white solid. ¹H NMR (400 MHz, DMSO-d⁶) δ (ppm) 7.69 (s, 1H), 7.54 (s, 1H), 7.34 (dd, J=8.5, 5.6 Hz, 2H), 7.11 (t, J=8.9 Hz, 2H), 4.06 (d, J=11.5 Hz, 3H), 3.82 (d, J=10.7 Hz, 2H), 3.57 (d, J=16.3 Hz, 1H), 3.54-3.45 (m, 2H), 3.23 (t, J=9.8 Hz, 1H), 3.12 (tt, J=7.4, 3.9 Hz, 2H), 2.99-2.78 (m, 4H), 2.73 (d, J=10.6 Hz, 1H), 2.61 (d, J=8.9 Hz, 2H), 2.41 (t, J=11.0 Hz, 1H), 2.28 (t, J=10.8 Hz, 1H), 2.16 (brs, 1H), 2.04-1.88 (m, 1H), 1.75 (d, J=11.4 Hz, 1H), 1.56 (s, 3H), 1.52 (s, 3H), 1.39 (t, J=7.3 Hz, 3H), 0.91 (d, J=6.2 Hz, 3H), 0.87 (t, J=7.0 Hz, 3H). MS (ESI) m/z 577.6 [M+H]⁺.

Example 5: 1-(4-(4-fluorobenzyl)-1-isopropyl-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 5)

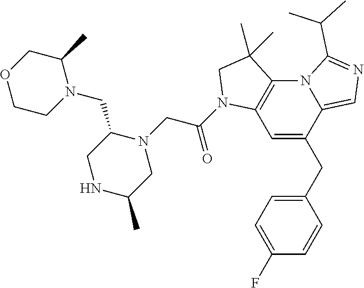 [SMILES: CC(C)c1ncc2c(Cc3ccc(F)cc3)cc3c(n12)C(C)(C)CN3C(=O)CN1C[C@@H](C)NC[C@@H]1CN1CCOC[C@H]1C]

Step 1: 2-chloro-1-(4-(4-fluorobenzyl)-1-isopropyl-8,8-dimethyl-7,8-dihydro-6H-imidazo[1,5-a]pyrrolo[2,3-e]pyridin-6-yl)ethan-1-one

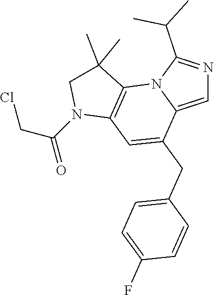 [SMILES: CC(C)c1ncc2c(Cc3ccc(F)cc3)cc3c(n12)C(C)(C)CN3C(=O)CCl]

In a flame-dried 100 mL round-bottomed flask, 1-(2-chloroacetyl)-6-(4-fluorobenzyl)-3,3-dimethyl-2,3-dihydro-1H-pyrrolo[3,2-b]pyridine-5-carbaldehyde (55 mg, 0.152 mmol, 1.0 eq) and ammonium acetate (70.5 mg, 0.915 mmol, 6.0 eq) were dissolved in acetic acid (1 mL) under nitrogen to give a solution. Isobutyraldehyde (55.0 mg, 0.762 mmol, 5.0 eq) was added to the reaction mixture dropwise at 22° C. The reaction mixture was stirred at 22° C. for 16 h, then saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:40 to give 2-chloro-1-(4-(4-fluorobenzyl)-1-isopropyl-8,8-dimethyl-7,8-dihydro-6H-imidazo[1,5-a]pyrrolo[2,3-e]pyridin-6-yl)ethan-1-one (50 mg, 79%) as an off-white solid. MS (ESI) m/z 415.4 [M+H]⁺.

Step 2: tert-butyl (2R,5S)-4-(2-(4-(4-fluorobenzyl)-1-isopropyl-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

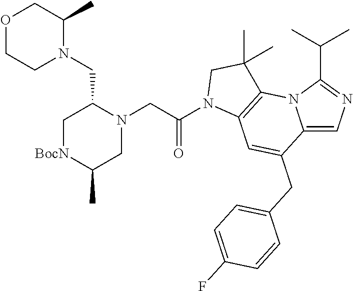 [SMILES: CC(C)c1ncc2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C]

In a flame-dried 50 mL round-bottomed flask, 2-chloro-1-(4-(4-fluorobenzyl)-1-isopropyl-8,8-dimethyl-7,8-dihydro-6H-imidazo[1,5-a]pyrrolo[2,3-e]pyridin-6-yl)ethan-1-one (50 mg, 0.121 mmol, 1.0 eq), potassium iodide (40.1 mg, 0.242 mmol, 2.0 eq) and K₂CO₃ (50.1 mg, 0.362 mmol, 3.0 eq) were dissolved in acetonitrile (5 mL) under nitrogen to give a solution. Tert-butyl (2R,5S)-2-methyl-5-(((R)-3-methylmorpholino)methyl)piperazine-1-carboxylate II (38 mg, 0.13 mmol, 1.1 eq) was added to the reaction mixture at 0° C. The reaction mixture was stirred at r.t. for 3 h. Saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:20 to give tert-butyl (2R,5S)-4-(2-(4-(4-fluorobenzyl)-1-isopropyl-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (50 mg, 59.9%) as an off-white solid. MS (ESI) m/z 691.7 [M+H]⁺.

Step 3: 1-(4-(4-fluorobenzyl)-1-isopropyl-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

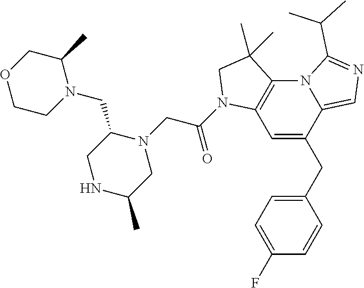 [SMILES: CC(C)c1ncc2c(Cc3ccc(F)cc3)cc3c(n12)C(C)(C)CN3C(=O)CN1C[C@@H](C)NC[C@@H]1CN1CCOC[C@H]1C]

In a flame-dried 50 mL round-bottomed flask, tert-butyl (2R,5S)-4-(2-(4-(4-fluorobenzyl)-1-isopropyl-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (50 mg, 0.072 mmol, 1.0 eq) was dissolved in 1,4-Dioxane (1 mL) under nitrogen to give a solution. HCl (1 mL, 4M in 1,4-Dioxane) was added to the reaction mixture at 22° C. The reaction mixture was stirred at 22° C. for 2 h. saturated NaHCO₃ (10 mL) was added to the reaction mixture followed by extraction with dichloromethane (10 mL×3). The combined organic lay ers were dried over Na₂SO₄, filtered and concentrated. The crude product was added to a silica gel column and was eluted with methanol/dichloromethane=1:20 to give 1-(4-(4-fluorobenzyl)-1-isopropyl-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (6.5 mg, 15.20%) as an off-white solid. ¹H NMR (400 MHz, DMSO-d₆) δ (ppm) 7.68 (s, 1H), 7.62 (s, 1H), 7.39 (dd, J=8.5, 5.7 Hz, 2H), 7.16 (dd, J=12.3, 5.5 Hz, 2H), 4.13 (d, J=10.4 Hz, 1H), 4.07 (s, 2H), 3.86 (t, J=13.8 Hz, 2H), 3.71-3.58 (m, 2H), 3.54 (d, J=11.1 Hz, 2H), 3.27 (t, J=10.4 Hz, 1H), 3.03-2.94 (m, 2H), 2.88 (dd, J=21.9, 12.7 Hz, 2H), 2.77 (d, J=9.9 Hz, 1H), 2.65 (d, J=8.9 Hz, 2H), 2.46 (t, J=10.9 Hz, 1H), 2.33 (t, J=11.1 Hz, 1H), 2.20 (brs, 1H), 2.09-1.92 (m, 1H), 1.80 (d, J=11.3 Hz, 1H), 1.60 (d, J=7.9 Hz, 6H), 1.39 (d, J=6.6 Hz, 3H), 1.35 (d, J=6.6 Hz, 3H), 0.94 (dd, J=10.8, 6.2 Hz, 6H). MS (ESI) m/z 591.6 [M+H]⁺.

Example 6: 1-(1-cyclobutyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 6)

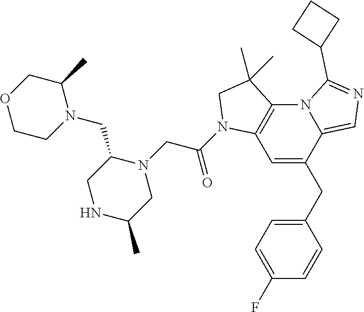 [SMILES: C[C@@H]1CN(CC(=O)N2CC(C)(C)c3c2cc(Cc2ccc(F)cc2)c2cnc(C4CCC4)n32)[C@@H](CN2CCOC[C@H]2C)CN1]

Step 1: 2-chloro-1-(1-cyclobutyl-4-(4-fluorobenzyl)-8,8-dimethyl-7,8-dihydro-6H-imidazo[1,5-a]pyrrolo[2,3-e]pyridin-6-yl)ethan-1-one

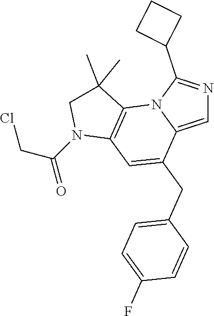 [SMILES: CC1(C)CN(C(=O)CCl)c2cc(Cc3ccc(F)cc3)c3cnc(C4CCC4)n3c21]

In a flame-dried 100 mL round-bottomed flask, 1-(2-chloroacetyl)-6-(4-fluoro benzyl)-3,3-dimethyl-2,3-dihydro-1H-pyrrolo[3,2-b]pyridine-5-carbaldehyde (45 mg, 0.125 mmol, 1.0 eq) and ammonium acetate (57.7 mg, 0.748 mmol, 6.0 eq) were dissolved in acetic acid (1 mL) under nitrogen to give a solution. Cyclo butanecarbaldehyde (52.5 mg, 0.624 mmol, 5.0 eq) was added to the reaction mixture dropwise at 22° C. The reaction mixture was stirred at 22° C. for 16 h, then saturated NaHCO₃ (10 mL) was added to the reaction mixture followed by extraction with ethyl acetate (15 mL×3). The combined organic layers we re dried over Na₂SO₄, filtered and concentrated. The crude product was added to a silica gel column and was eluted with methanol/dichloromethane=1:40 to give 2-chloro-1-(1-cyclobutyl-4-(4-fluorobenzyl)-8,8-dimethyl-7,8-dihydro-6H-imidazo[1,5-a]pyrrolo[2,3-e]pyridin-6-yl)ethan-1-one (40 mg, 75%) as an off-white solid. MS (ESI) m/z 427.4 [M+H]⁺.

Step 2: tert-butyl (2R,5S)-4-(2-(1-cyclobutyl-4-(4-fluorobenzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

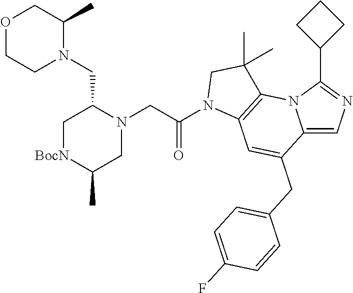 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1cnc(C3CCC3)n21]

In a flame-dried 50 mL round-bottomed flask, 2-chloro-1-(1-cyclobutyl-4-(4-fluorobenzyl)-8,8-dimethyl-7,8-dihydro-6H-imidazo[1,5-a]pyrrolo[2,3-e]pyridin-6-yl) ethan-1-one II (40 mg, 0.094 mmol, 1.0 eq), potassium iodide (31.2 mg, 0.188 mmol, 2.0 eq) and K₂CO₃ (38.9 mg, 0.282 mmol, 3.0 eq) were dissolved in acetonitrile (5 mL) under nitrogen to give a solution. tert-butyl (2R,5S)-2-methyl-5-(((R)-3-methylmorpholino)methyl)piperazine-1-carboxylate (30 mg, 0.10 mmol, 1.1 eq) was added to the reaction mixture at 0° C. The reaction mixture was stirred at r.t. for 3 h. Saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:20 to give tert-butyl (2R,5S)-4-(2-(1-cyclobutyl-4-(4-fluorobenzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (35 mg, 53.0%) as an off-white solid. MS (ESI) m/z 703.8 [M+H]⁺.

Step 3: 1-(1-cyclobutyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

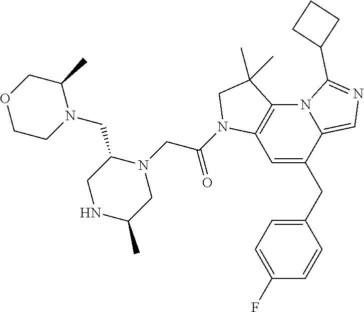 [SMILES: C[C@@H]1CN(CC(=O)N2CC(C)(C)c3c2cc(Cc2ccc(F)cc2)c2cnc(C4CCC4)n32)[C@@H](CN2CCOC[C@H]2C)CN1]

In a flame-dried 50 mL round-bottomed flask, tert-butyl (2R,5S)-4-(2-(1-cyclobutyl-4-(4-fluorobenzyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (45 mg, 0.064 mmol, 1.0 eq) was dissolved in 1,4-Dioxane (1 mL) under nitrogen to give a solution. HCl (1 mL, 4M in 1,4-Dioxane) was added to the reaction mixture at 22° C. The reaction mixture was stirred at 22° C. for 2 h. Saturated NaHCO₃ (10 mL) was added to the reaction mixture followed by extraction with dichloromethane (10 mL×3). The combined organic layers were dried over Na₂SO₄, filtered and concentrated. The crude product was added to a silica gel column and was eluted with methanol/dichloromethane=1:20 to give 1-(1-cyclobutyl-4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (9.5 mg, 24.62%) as an off-white solid. ¹H NMR (400 MHz, DMSO-d₆) δ (ppm) 7.67 (s, 1H), 7.63 (s, 1H), 7.34 (dd, J=8.5, 5.6 Hz, 2H), 7.12 (t, J=8.9 Hz, 2H), 4.15-3.98 (m, 4H), 3.86-3.73 (m, 2H), 3.60 (d, J=16.4 Hz, 1H), 3.50 (d, J=11.2 Hz, 2H), 3.21 (t, J=10.2 Hz, 1H), 3.01-2.84 (m, 3H), 2.78 (dd, J=23.7, 12.0 Hz, 2H), 2.62 (d, J=9.1 Hz, 2H), 2.41 (ddd, J=11.5, 10.6, 6.8 Hz, 3H), 2.35-2.25 (m, 1H), 2.16 (brs, 1H), 1.96 (td, J=14.4, 8.0 Hz, 3H), 1.76 (d, J=11.2 Hz, 1H), 1.57 (d, J=8.2 Hz, 6H), 0.90 (t, J=6.2 Hz, 6H). MS (ESI) m/z 603.6 [M+H]⁺.

Example 7: 1-(4-(4-fluorobenzyl)-8,8-dimethyl-1-(tetrahydro-2H-pyran-4-yl)-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 7)

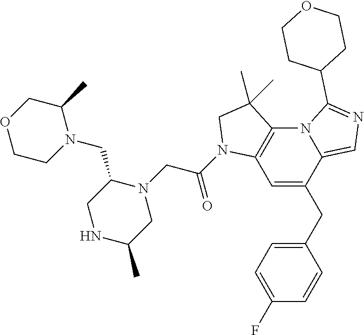 [SMILES: C[C@@H]1CN(CC(=O)N2CC(C)(C)c3c2cc(Cc2ccc(F)cc2)c2cnc(C4CCOCC4)n32)[C@@H](CN2CCOC[C@H]2C)CN1]

Step 1: 2-chloro-1-(4-(4-fluorobenzyl)-8,8-dimethyl-1-(tetrahydro-2H-pyran-4-yl)-7,8-dihydro-6H-imidazo[1,5-a]pyrrolo[2,3-e]pyridin-6-yl)ethan-1-one

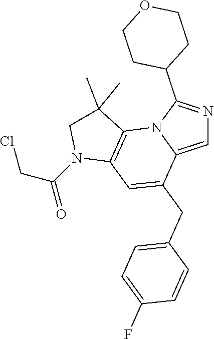 [SMILES: CC1(C)CN(C(=O)CCl)c2cc(Cc3ccc(F)cc3)c3cnc(C4CCOCC4)n3c21]

In a flame-dried 100 mL round-bottomed flask, 1-(2-chloroacetyl)-6-(4-fluoro benzyl)-3,3-dimethyl-2,3-dihydro-1H-pyrrolo[3,2-b]pyridine-5-carbaldehyde (45 mg, 0.125 mmol, 1.0 eq) and ammonium acetate (57.7 mg, 0.748 mmol, 6.0 eq) were dissolved in acetic acid (1 mL) under nitrogen to give a solution. Tetrahydro-2H-pyran-4-carbaldehyde (71.2 mg, 0.624 mmol, 5.0 eq) was added to the reaction mixture dropwise at 22° C. The reaction mixture was stirred at 22° C. for 16 h, then saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:40 to give 2-chloro-1-(4-(4-fluorobenzyl)-8,8-dimethyl-1-(tetrahydro-2H-pyran-4-yl)-7,8-dihydro-6H-imidazo[1,5-a]pyrrolo[2,3-e]pyridin-6-yl)ethan-1-one (20 mg, 35.2%) as an off-white solid. MS (ESI) m/z 456.3 [M+H]⁺.

Step 2: tert-butyl (2R,5S)-4-(2-(4-(4-fluorobenzyl)-8,8-dimethyl-1-(tetrahydro-2H-pyran-4-yl)-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

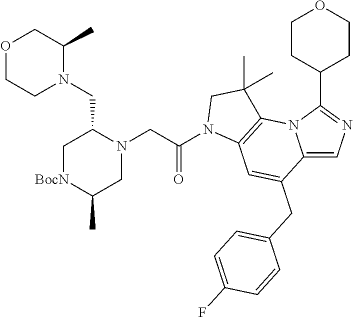 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1cnc(C3CCOCC3)n21]

In a flame-dried 50 mL round-bottomed flask, 2-chloro-1-(4-(4-fluorobenzyl)-8,8-dimethyl-1-(tetrahydro-2H-pyran-4-yl)-7,8-dihydro-6H-imidazo[1,5-a]pyrrolo[2,3-e]pyridin-6-yl)ethan-1-one (20 mg, 0.044 mmol, 1.0 eq), potassium iodide (10.9 mg, 0.066 mmol, 1.5 eq) and K₂CO₃ (18.2 mg, 0.132 mmol, 3.0 eq) were dissolved in acetonitrile (5 mL) under nitrogen to give a solution. Tert-butyl (2R,5S)-2-methyl-5-(((R)-3-methylmorpholino)methyl)piperazine-1-carboxylate II (15.1 mg, 0.048 mmol, 1.1 eq) was added to the reaction mixture at 0 T. The re action mixture was stirred at r.t. for 3 h. saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:20 to give tert-butyl (2R,5S)-4-(2-(4-(4-fluorobenzyl)-8,8-dimethyl-1-(tetrahydro-2H-pyran-4-yl)-7,8-dihydro-6H-imidazo[1,5-a]pyrrolo[2,3-e]pyridin-6-yl)-2-oxoethyl)-2-methyl-5-(((R)-3-methylmorpholino)methyl)piper azine-1-carboxylate (20 mg, 62.2%) as an off-white solid. MS (ESI) m/z 733.6 [M+H]⁺.

Step 3: 1-(4-(4-fluorobenzyl)-8,8-dimethyl-1-(tetrahydro-2H-pyran-4-yl)-7,8-di hydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

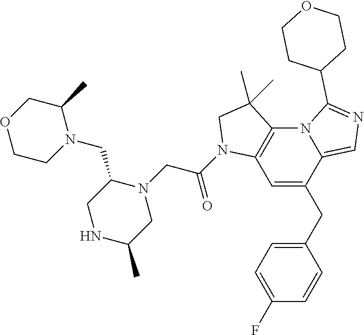 [SMILES: C[C@@H]1CN(CC(=O)N2CC(C)(C)c3c2cc(Cc2ccc(F)cc2)c2cnc(C4CCOCC4)n32)[C@@H](CN2CCOC[C@H]2C)CN1]

In a flame-dried 50 mL round-bottomed flask, tert-butyl (2R,5S)-4-(2-(4-(4-fluorobenzyl)-8,8-dimethyl-1-(tetrahydro-2H-pyran-4-yl)-7,8-dihydro-6H-imidazo[1, 5-a]pyrrolo[2,3-e]pyridin-6-yl)-2-oxoethyl)-2-methyl-5-(((R)-3-methylmorpholino)methyl)piperazine-1-carboxylate (25 mg, 0.034 mmol, 1.0 eq) was dissolved in 1,4-Dioxane (1 mL) under nitrogen to give a solution. HCl (1 mL, 4M in 1,4-Dioxane) was added to the reaction mixture at 22° C. The reaction mixture was stirred at 22° C. for 2 h. Saturated NaHCO₃ (10 mL) was added to the reaction mixture followed by extraction with dichloromethane (10 mL×3). The combined organic layers were dried over Na₂SO₄, filtered and concentrated. The crude product was added to a silica gel column and was eluted with methanol/dichloromethane=1:20 to give 1-(4-(4-fluorobenzyl)-8,8-dimethyl-1-(tetrahydro-2H-pyran-4-yl)-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (5 mg, 23.16%) as an off-white solid. ¹H NMR (400 MHz, DMSO-d₆) δ (ppm) 7.67 (s, 1H), 7.62 (s, 1H), 7.35 (dd, J=8.4, 5.7 Hz, 2H), 7.12 (t, J=8.8 Hz, 2H), 4.14-4.02 (m, 2H), 3.97 (d, J=10.6 Hz, 2H), 3.90-3.75 (m, 2H), 3.68-3.41 (m, 6H), 3.21 (t, J=10.0 Hz, 1H), 3.06-2.72 (m, 6H), 2.71-2.58 (m, 3H), 2.36-2.25 (m, 1H), 2.15 (brs, 1H), 2.11-1.90 (m, 3H), 1.85-1.65 (m, 3H), 1.58 (d, J=8.3 Hz, 6H), 0.98-0.84 (m, 6H). MS (ESI) m/z 633.5 [M+H]⁺.

Example 8: 1-(4-(4-fluorobenzyl)-1-(4-fluorophenyl)-8,8-dimethyl-7,8-di hydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 8)

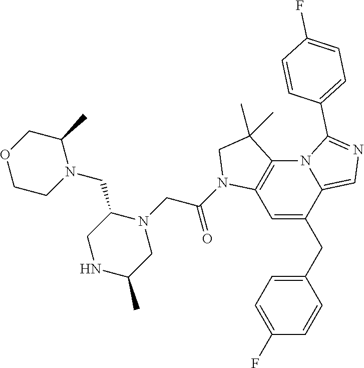 [SMILES: C[C@@H]1CN(CC(=O)N2CC(C)(C)c3c2cc(Cc2ccc(F)cc2)c2cnc(-c4ccc(F)cc4)n32)[C@@H](CN2CCOC[C@H]2C)CN1]

Step 1: 2-chloro-1-(4-(4-fluorobenzyl)-1-(4-fluorophenyl)-8,8-dimethyl-7,8-dihydro-6H-imidazo[1,5-a]pyrrolo[2,3-e]pyridin-6-yl)ethan-1-one

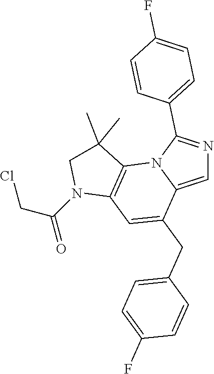 [SMILES: CC1(C)CN(C(=O)CCl)c2cc(Cc3ccc(F)cc3)c3cnc(-c4ccc(F)cc4)n3c21]

In a flame-dried 100 mL round-bottomed flask, 1-(2-chloroacetyl)-6-(4-fluorobenzyl)-3,3-dimethyl-2,3-dihydro-1H-pyrrolo[3,2-b]pyridine-5-carbaldehyde (50 mg, 0.139 mmol, 1.0 eq) and ammonium acetate (64.1 mg, 0.831 mmol, 6.0 eq) were dissolved in acetic acid (1 mL) under nitrogen to give a solution. 4-Fluorobenzaldehyde (86 mg, 0.693 mmol, 5.0 eq) was added to the reaction mixture dropwise at 22° C. The reaction mixture was stirred at 22° C. for 16 h, then saturated NaHCO₃ (10 mL) was added to the reaction mixture followed by extraction with ethyl acetate (15 mL×3). The combined organic layers were dried over Na₂SO₄, filtered and concentrated. The crude product was added to a silica gel column and was eluted with methanol/dichloromethane=1:40 to give 2-chloro-1-(4-(4-fluorobenzyl)-1-(4-fluorophenyl)-8,8-dimethyl-7,8-dihydro-6H-imidazo[1,5-a]pyrrolo[2,3-e]pyridin-6-yl)ethan-1-one (35 mg, 54%) as an off-white solid. MS (ESI) m/z 466.3 [M+H]⁺.

Step 2: tert-butyl (2R,5S)-4-(2-(4-(4-fluorobenzyl)-1-(4-fluorophenyl)-8,8-dim ethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

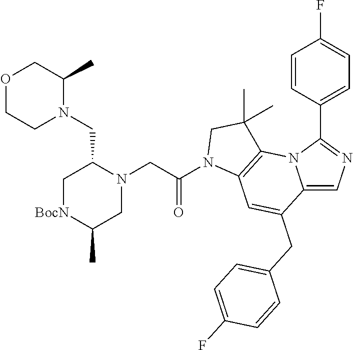 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1cnc(-c3ccc(F)cc3)n21]

In a flame-dried 50 mL round-bottomed flask, 2-chloro-1-(4-(4-fluorobenzyl)-1-(4-fluorophenyl)-8,8-dimethyl-7,8-dihydro-6H-imidazo[1,5-a]pyrrolo[2,3-e]pyridin-6-yl)ethan-1-one (30 mg, 0.064 mmol, 1.0 eq), potassium iodide (21.38 mg, 0.129 mmol, 2.0 eq) and K₂CO₃ (26.7 mg, 0.193 mmol, 3.0 eq) were dissolved in acetonitrile (5 mL) under nitrogen to give a solution. Tert-butyl (2R,5S)-2-methyl-5-(((R)-3-methylmorpholino)methyl)piperazine-1-carboxylate (21 mg, 0.07 mmol, 1.1 eq) was added to the reaction mixture at 0° C. The reaction mixture was stirred at r.t. for 3 h. Saturated NaHCO₃ (10 mL) was added to the re action mixture followed by extraction with ethyl acetate (15 mL×3). The combined organic layers were dried over Na₂SO₄, filtered and concentrated. The crude product was added to a silica gel column and was eluted with methanol/dichloromethane=1:20 to give tert-butyl (2R,5S)-4-(2-(4-(4-fluorobenzyl)-1-(4-fluorophenyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (20 mg, 41.8%) as an off-white solid. MS (ESI) m/z 743.8 [M+H]⁺.

Step 3: 1-(4-(4-fluorobenzyl)-1-(4-fluorophenyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

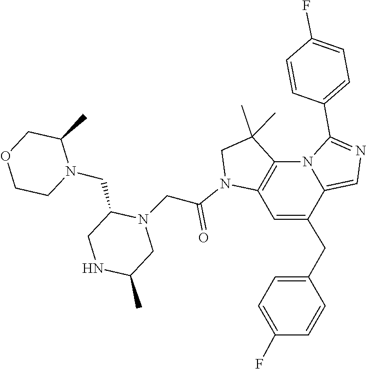 [SMILES: C[C@@H]1CN(CC(=O)N2CC(C)(C)c3c2cc(Cc2ccc(F)cc2)c2cnc(-c4ccc(F)cc4)n32)[C@@H](CN2CCOC[C@H]2C)CN1]

In a flame-dried 50 mL round-bottomed flask, tert-butyl (2R,5S)-4-(2-(4-(4-fluorobenzyl)-1-(4-fluorophenyl)-8,8-dimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (40 mg, 0.054 mmol, 1.0 eq) was dissolved in 1,4-Dioxane (1.0 mL) under nitrogen to give a solution. HCl (1 mL, 4M in 1,4-Dioxane) was added to the reaction mixture at 22° C. The reaction mixture was stirred at 22° C. for 2 h. saturated NaHCO₃ (10 mL) was added to the reaction mixture followed by extraction with dichloromethane (10 mL×3). The combined organic layers were dried over Na₂SO₄, filtered and concentrated. The crude product was added to a silica gel column and was eluted with methanol/dichloromethane=1:20 to give 1-(4-(4-fluorobenzyl)-1-(4-fluorophenyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (8 mg, 23%) as an off-white solid. ¹H NMR (400 MHz, DMSO-d₆) δ (ppm) 7.78 (s, 1H), 7.73 (s, 1H), 7.62 (dd, J=8.4, 5.6 Hz, 2H), 7.41 (dd, J=8.4, 5.7 Hz, 2H), 7.32 (t, J=8.8 Hz, 2H), 7.15 (t, J=8.9 Hz, 2H), 4.12 (s, 2H), 3.81 (dd, J=17.1, 14.0 Hz, 2H), 3.58 (dd, J=18.5, 13.3 Hz, 2H), 3.48 (t, J=8.9 Hz, 2H), 3.20 (t, J=9.9 Hz, 1H), 2.90 (t, J=9.5 Hz, 2H), 2.85-2.76 (m, 2H), 2.69 (d, J=12.6 Hz, 1H), 2.58 (d, J=8.9 Hz, 2H), 2.40 (t, J=11.3 Hz, 1H), 2.25 (t, J=10.9 Hz, 1H), 2.12 (brs, 1H), 2.00 (dd, J=14.1, 6.6 Hz, 1H), 1.91 (t, J=10.0 Hz, 1H), 1.72 (d, J=11.3 Hz, 1H), 0.92 (s, 3H), 0.87 (s, 3H), 0.86 (s, 3H), 0.77 (s, 3H). MS (ESI) m/z 643.6 [M+H]⁺.

Example 9: 1-(4-(4-fluorobenzyl)-8,8-dimethyl-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (Compound 9)

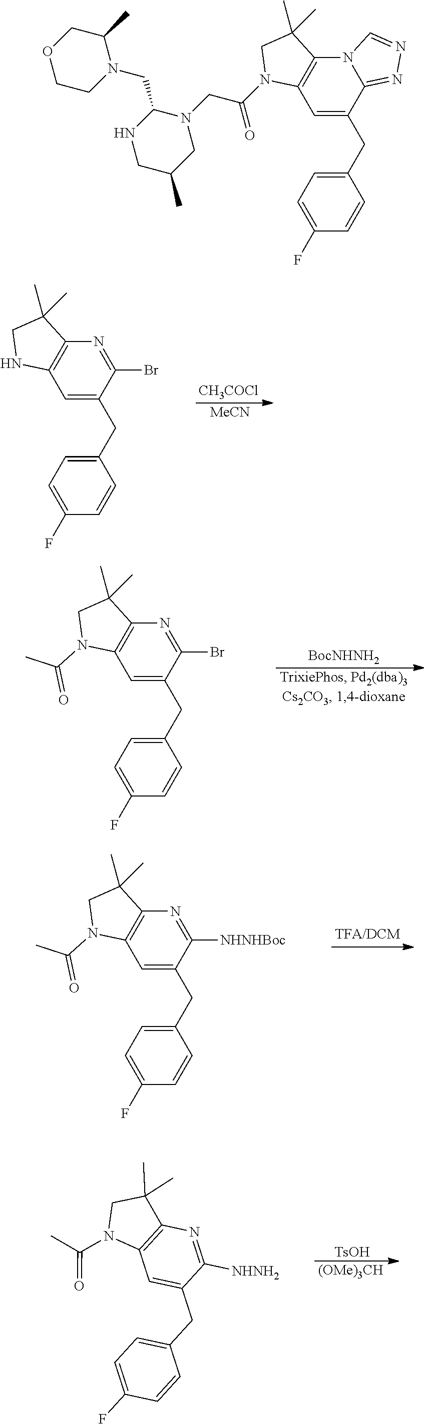

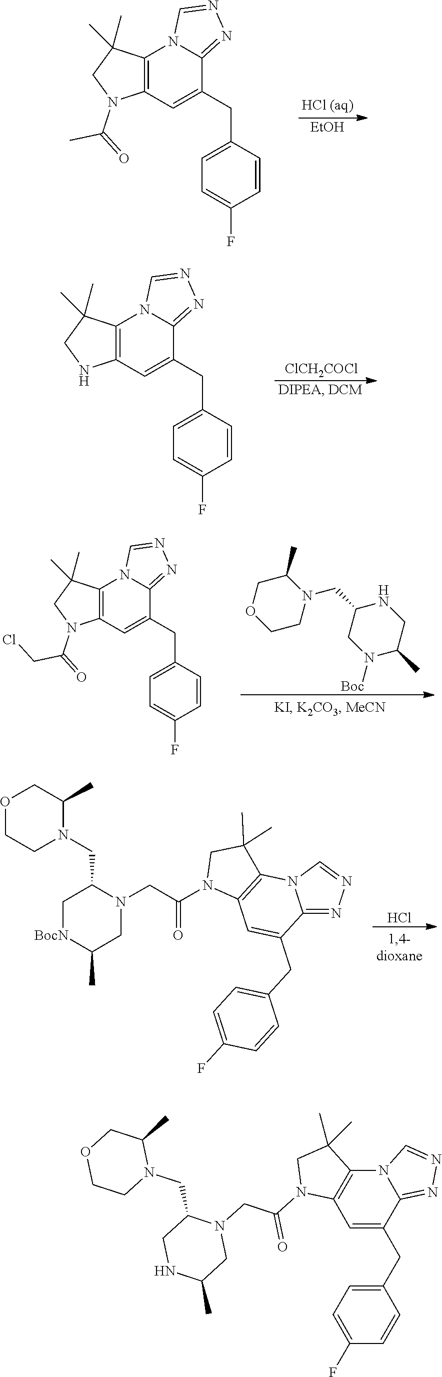

Step 1: 1-(5-bromo-6-(4-fluorobenzyl)-3,3-dimethyl-2,3-dihydro-1H-pyrrolo[3,2-b]pyridin-1-yl)ethan-1-one

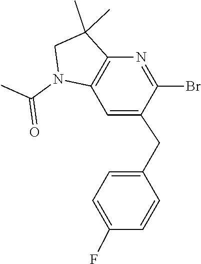 [SMILES: CC(=O)N1CC(C)(C)c2nc(Br)c(Cc3ccc(F)cc3)cc21]

To a solution of 5-bromo-6-(4-fluorobenzyl)-3,3-dimethyl-2,3-dihydro-1H-pyrrolo[3,2-b]pyridine (1 g, 2.98 mmol, 1.0 eq) in acetonitrile (20 ml) was added dropwise acetyl chloride (0.281 g, 3.58 mmol) at 0° C. The mixture was stirred at r.t. for 1 h. The mixture was quenched by sat·NaHCO₃·aq. and diluted with ethyl acetate and then washed with water and saturated brine. The organic layer was dried over Na₂SO₄, filtered and concentrated. The crude product was added to a silica gel column and was eluted with ethyl acetate/hexane (ethyl acetate:hexane=1:3) to give 1-(5-bromo-6-(4-fluorobenzyl)-3,3-dimethyl-2,3-dihydro-1H-pyrrolo[3,2-b]pyridin-1-yl)ethan-1-one (1.0 g, 89%) as a white solid. MS (ESI) m/z 377.1 [M+H]⁺.

Step 2: tert-butyl 2-(1-acetyl-6-(4-fluorobenzyl)-3,3-dimethyl-2,3-dihydro-1H-pyrrolo[3,2-b]pyridin-5-yl)hydrazine-1-carboxylate

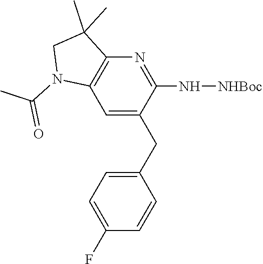 [SMILES: CC(=O)N1CC(C)(C)c2nc(NNC(=O)OC(C)(C)C)c(Cc3ccc(F)cc3)cc21]

To a solution of 1-(5-bromo-6-(4-fluorobenzyl)-3,3-dimethyl-2,3-dihydro-1H-pyrrolo[3,2-b]pyridin-1-yl)ethan-1-one (600 mg, 1.59 mmol) in 1,4-Dioxane (10 ml) was added tert-butyl hydrazinecarboxylate (252 mg, 1.91 mmol), Cs₂CO₃ (1555 mg, 4.77 mmol), [1,1′-binaphthalen]-2-yldi-tert-butylphosphane (127 mg, 0.32 mmol) and Pd₂(dba)₃ (146 mg, 0.16 mmol). The mixture was filled with N₂ three times and stirred at 130° C. for 2 h. The mixture was cooled to r.t. and diluted with ethyl acetate and then washed with water and saturated brine. The organic layer was dried over Na₂SO₄, filtered and concentrated. The crude product was added to a silica gel column and was eluted with ethyl acetate/hexane (ethyl acetate:hexane=7:10) to give tert-butyl 2-(1-acetyl-6-(4-fluorobenz yl)-3,3-dimethyl-2,3-dihydro-1H-pyrrolo[3,2-b]pyridin-5-yl)hydrazine-1-carboxylate (0.36 g, 52.8%) as a white solid. MS (ESI) m/z 429.2 [M+H]⁺.

Step 3: 1-(6-(4-fluorobenzyl)-5-hydrazineyl-3,3-dimethyl-2,3-dihydro-1H-pyrrolo[3,2-b]pyridin-1-yl)ethan-1-one (Intermediate 1)

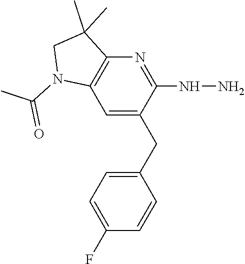 [SMILES: CC(=O)N1CC(C)(C)c2nc(NN)c(Cc3ccc(F)cc3)cc21]

To a solution of tert-butyl 2-(1-acetyl-6-(4-fluorobenzyl)-3,3-dimethyl-2,3-dihydro-1H-pyrrolo[3,2-b]pyridin-5-yl)hydrazine-1-carboxylate (0.36 g, 0.84 mmol) in DCM (1 ml) was added TFA (1 ml). The mixture was stirred at r.t. for 1 h. The mixture was concentrated to give crude product 280 mg, which was used next step directly. MS (ESI) m/z 329.2 [M+H]⁺.

Step 4: 1-(4-(4-fluorobenzyl)-8,8-dimethyl-7,8-dihydro-6H-pyrrolo[2,3-e][1,2,4]triazolo[4,3-a]pyridin-6-yl)ethan-1-one

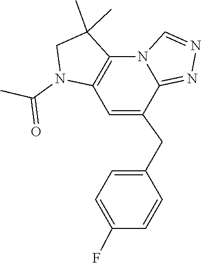 [SMILES: CC(=O)N1CC(C)(C)c2c1cc(Cc1ccc(F)cc1)c1nncn21]

To a solution of 1-(6-(4-fluorobenzyl)-5-hydrazinyl-3,3-dimethyl-2,3-dihydro-1H-pyrrolo[3,2-b]pyridin-1-yl)ethan-1-one (90 mg, 0.27 mmol) in trimethoxymethane (2 ml) was added 4-methylbenzenesulfonic acid (47.2 mg, 0.27 mmol). The mixture was stirred 50° C. for 1 h. The mixture was cooled to r.t and concentrated. The crude product was added to a silica gel column and eluted with methanol/dichloromethane (methanol:dichloromethane=1:20) to give 1-(4-(4-fluorobenzyl)-8,8-dimethyl-7,8-dihydro-6H-pyrrolo[2,3-e][1,2,4]triazolo[4,3-a]pyridin-6-yl)ethan-1-one (40 mg, 43.1%) as a yellow oil. MS (ESI) m/z 339.2 [M+H]⁺.

Step 5: 4-(4-fluorobenzyl)-8,8-dimethyl-7,8-dihydro-6H-pyrrolo[2,3-e][1,2,4]triazolo[4,3-a]pyridine

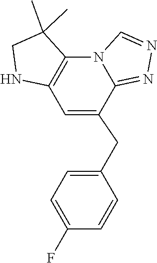 [SMILES: CC1(C)CNc2cc(Cc3ccc(F)cc3)c3nncn3c21]

To a solution of 1-(4-(4-fluorobenzyl)-8,8-dimethyl-7,8-dihydro-6H-pyrrolo[2,3-e][1,2,4]triazolo[4,3-a]pyridin-6-yl)ethan-1-one (40 mg, 0.12 mmol) in EtOH (2 ml) was added 5 M HCl (2 ml). The mixture was stirred at 95° C. for 6 h. The mixture was concentrated and diluted with ethyl acetate, washed with saturated NaHCO₃(aq) and brine. The organic layer was dried over Na₂SO₄, filtered and concentrated to give crude product 30 mg, which was used next step directly. MS (ESI) m/z 297.1 [M+H]⁺.

Step 6: 2-chloro-1-(4-(4-fluorobenzyl)-8,8-dimethyl-7,8-dihydro-6H-pyrrolo[2,3-e][1,2,4]triazolo[4,3-a]pyridin-6-yl)ethan-1-one

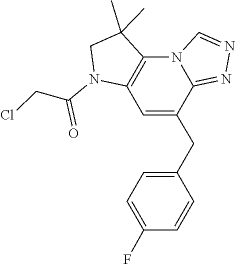 [SMILES: CC1(C)CN(C(=O)CCl)c2cc(Cc3ccc(F)cc3)c3nncn3c21]

To a solution of 4-(4-fluorobenzyl)-8,8-dimethyl-7,8-dihydro-6H-pyrrolo[2,3-e][1,2,4]triazolo[4,3-a]pyridine (30 mg, 0.1 mmol) in DCM (2 ml) was added N-ethyl-N-isopropylpropan-2-amine (26.2 mg, 0.2 mmol) and 2-chloroacetyl chloride (17.1 mg, 0.15 mmol). The mixture was stirred at r.t. for 1 h. After that the mixture was concentrated to give crude product, which was added to a silica gel column and eluted with ethyl acetate/hexane (ethyl acetate:hexane=1:3) to give 2-chloro-1-(4-(4-fluorobenzyl)-8,8-dimethyl-7,8-dihydro-6H-pyrrolo[2,3-e][1,2,4]triazolo[4,3-a]pyridin-6-yl)ethan-1-one (31 mg, 82%) as a yellow oil. MS (ESI) m/z 373.1 [M+H]⁺.

Step 7: tert-butyl (2R,5S)-4-(2-(4-(4-fluorobenzyl)-8,8-dimethyl-7,8-dihydro-6H-pyrrolo[2,3-e][1,2,4]triazolo[4,3-a]pyridin-6-yl)-2-oxoethyl)-2-methyl-5-(((R)-3-m ethylmorpholino)methyl)piperazine-1-carboxylate

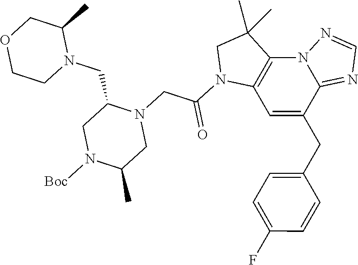 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1ncnn21]

To a solution of 2-chloro-1-(4-(4-fluorobenzyl)-8,8-dimethyl-7,8-dihydro-6H-pyrrolo[2,3-e][1,2,4]triazolo[4,3-a]pyridin-6-yl)ethan-1-one (31 mg, 0.08 mmol) in acetonitrile (2 ml) was added tert-butyl (2R,5S)-2-methyl-5-(((R)-3-methylmorpholino)methyl)piperazine-1-carboxylate (26.1 mg, 0.08 mmol), potassium iodide (20.7 mg, 0.12 mmol) and K₂CO₃ (34.5 mg, 0.24 mmol). The mixture was stir red at 29° C. for 2 h. The mixture was diluted with ethyl acetate, washed with water and saturated brine. The organic layer was dried over Na₂SO₄, filtered and concentrated to give yellow oil, which was added to a silica gel column and eluted with DCM/MeOH (DCM:MeOH=10:1) to afford the title compound (36 mg, 66%) as a white solid. MS (ESI) m/z 650.4 [M+H]⁺.

Step 8: 1-(4-(4-fluorobenzyl)-8,8-dimethyl-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl) piperazin-1-yl)ethan-1-one

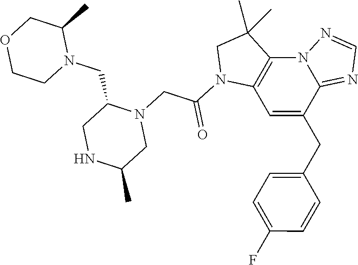 [SMILES: C[C@@H]1CN(CC(=O)N2CC(C)(C)c3c2cc(Cc2ccc(F)cc2)c2ncnn32)[C@@H](CN2CCOC[C@H]2C)CN1]

To a solution of tert-butyl (2R,5S)-4-(2-(4-(4-fluorobenzyl)-8,8-dimethyl-7,8-dihydro-6H-pyrrolo[2,3-e][1,2,4]triazolo[4,3-a]pyridin-6-yl)-2-oxoethyl)-2-methyl-5-(((R)-3-methylmorpholino)methyl)piperazine-1-carboxylate (36 mg, 0.06 mmol) in DCM (2 ml) was added TFA (2 ml). The mixture was stirred at r.t. for 1 h. The mixture was concentrated and diluted with ethyl acetate, washed with Sat. NaHCO₃·aq and brine. The organic layer was dried over Na₂SO₄, filtered and concentrated to give yellow oil, which was added to a Prep HPLC column and eluted with water/acetonitrile (5%-50%) to give 1-(4-(4-fluorobenzyl)-8,8-dimethyl-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (5.7 mg, 18.72%) as a white solid. ¹H NMR (400 MHz, CD₃OD) δ (ppm) 9.44 (s, 1H), 8.22 (s, 1H), 7.36 (dd, J=8.5, 5.4 Hz, 2H), 7.02 (t, J=8.8 Hz, 2H), 4.45-4.25 (m, 2H), 4.15-3.93 (m, 3H), 3.71-3.50 (m, 3H), 3.45-3.32 (m, 1H), 3.13-2.76 (m, 7H), 2.54-2.39 (m, 2H), 2.31-1.80 (m, 3H), 1.65 (s, 3H), 1.62 (s, 3H), 1.02 (d, J=6.3 Hz, 3H), 0.95 (d, J=6.3 Hz, 3H). MS (E SI) m/z 550.3 [M+H]⁺.

Example 10:1-(4-(4-fluorobenzyl)-1,8,8-trimethyl-7,8-dihydro-6H-pyrrolo[2,3-e][1,2,4]triazolo[4,3-a]pyridin-6-yl)-2-((2R,5R)-5-methyl-2-(((R)-3-methyl morpholino)methyl)piperazin-1-yl)ethan-1-one (Compound 10)

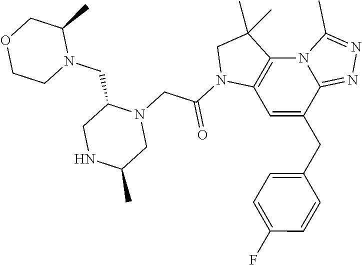 [SMILES: Cc1nnc2c(Cc3ccc(F)cc3)cc3c(n12)C(C)(C)CN3C(=O)CN1C[C@@H](C)NC[C@@H]1CN1CCOC[C@H]1C]

Following the synthesis of EXAMPLE 9, EXAMPLE 10 was obtained as a white solid (5.1 mg). ¹H NMR (400 MHz, CD₃OD) δ (ppm) 8.22 (s, 1H), 7.34 (dd, J=8.5, 5.4 Hz, 2H), 7.02 (t, J=8.8 Hz, 2H), 4.40-4.23 (m, 2H), 4.17-3.90 (m, 3H), 3.69-3.51 (m, 3H), 3.47-3.32 (m, 1H), 3.10-2.79 (m, 10H), 2.54-2.40 (m, 2H), 2.31-1.81 (m, 3H), 1.66 (s, 3H), 1.62 (s, 3H), 1.02 (d, J=6.3 Hz, 3H), 0.95 (d, J=6.3 Hz, 3H). MS (ESI) m/z 564.3 [M+H]⁺.

Example 11: 1-(4-(4-fluorobenzyl)-1-isopropyl-8,8-dimethyl-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (Compound 11)

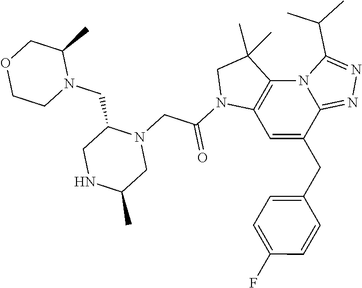 [SMILES: CC(C)c1nnc2c(Cc3ccc(F)cc3)cc3c(n12)C(C)(C)CN3C(=O)CN1C[C@@H](C)NC[C@@H]1CN1CCOC[C@H]1C]

Following the synthesis of EXAMPLE 9, EXAMPLE 11 was obtained as a white solid (2.9 mg). ¹H NMR (400 MHz, CD₃OD) δ (ppm) 8.20 (s, 1H), 7.35 (dd, J=8.6, 5.4 Hz, 2H), 7.02 (t, J=8.8 Hz, 2H), 4.40-4.23 (m, 2H), 4.17-3.93 (m, 3H), 3.89-3.76 (m, 1H), 3.66-3.54 (m, 3H), 3.44-3.33 (m, 1H), 3.09-2.77 (m, 7H), 2.55-2.40 (m, 2H), 2.28-1.81 (m, 3H), 1.68 (s, 3H), 1.66 (s, 3H), 1.53 (dd, J=6.7, 4.4 Hz, 6H), 1.02 (d, J=6.3 Hz, 3H), 0.95 (d, J=6.3 Hz, 3H). MS (ESI) m/z 592.4 [M+H]⁺.

Example 12: 1-(1-cyclopropyl-4-(4-fluorobenzyl)-8,8-dimethyl-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (Compound 12)

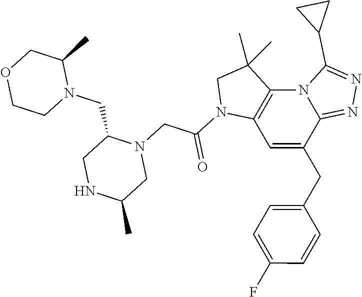 [SMILES: C[C@@H]1CN(CC(=O)N2CC(C)(C)c3c2cc(Cc2ccc(F)cc2)c2nnc(C4CC4)n32)[C@@H](CN2CCOC[C@H]2C)CN1]

Following the synthesis of EXAMPLE 9, EXAMPLE 12 was obtained as a white solid (9.5 mg). ¹H NMR (400 MHz, CD₃OD) δ (ppm) 8.21 (s, 1H), 7.34 (dd, J=8.5, 5.5 Hz, 2H), 7.01 (t, J=8.8 Hz, 2H), 4.37-4.21 (m, 2H), 4.16-3.95 (m, 3H), 3.69-3.53 (m, 3H), 3.38 (m, 1H), 3.10-2.77 (m, 7H), 2.53-2.38 (m, 3H), 2.33-1.82 (m, 3H), 1.75 (s, 3H), 1.71 (s, 3H), 1.37-1.24 (m, 4H), 1.02 (d, J=6.2 Hz, 3H), 0.95 (d, J=6.2 Hz, 3H). MS (ESI) m/z 590.4 [M+H]⁺.

Example 13: 1-(4-(4-fluorobenzyl)-8,8-dimethyl-1-(trifluoromethyl)-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (Compound 13)

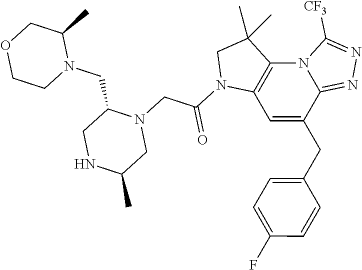 [SMILES: C[C@@H]1CN(CC(=O)N2CC(C)(C)c3c2cc(Cc2ccc(F)cc2)c2nnc(C(F)(F)F)n32)[C@@H](CN2CCOC[C@H]2C)CN1]

Following the synthesis of EXAMPLE 9, EXAMPLE 13 was obtained as a white solid (17.5 mg). ¹H NMR (400 MHz, CD₃OD) δ (ppm) 8.26 (s, 1H), 7.39 (dd, J=8.4, 5.4 Hz, 2H), 7.11 (t, J=8.7 Hz, 2H), 4.61-4.37 (m, 2H), 4.12-3.82 (m, 5H), 3.71-3.33 (m, 11H), 3.18-2.90 (m, 3H), 1.59 (s, 3H), 1.58 (s, 3H), 1.41-1.18 (m, 6H). MS (ESI) m/z 618.5 [M+H]⁺.

Example 14: 1-(4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 14)

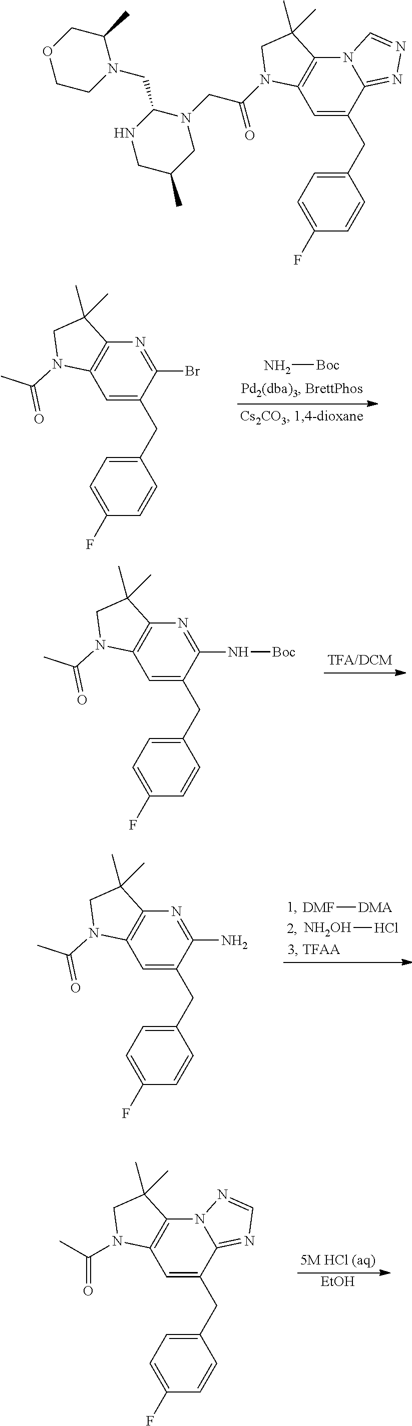

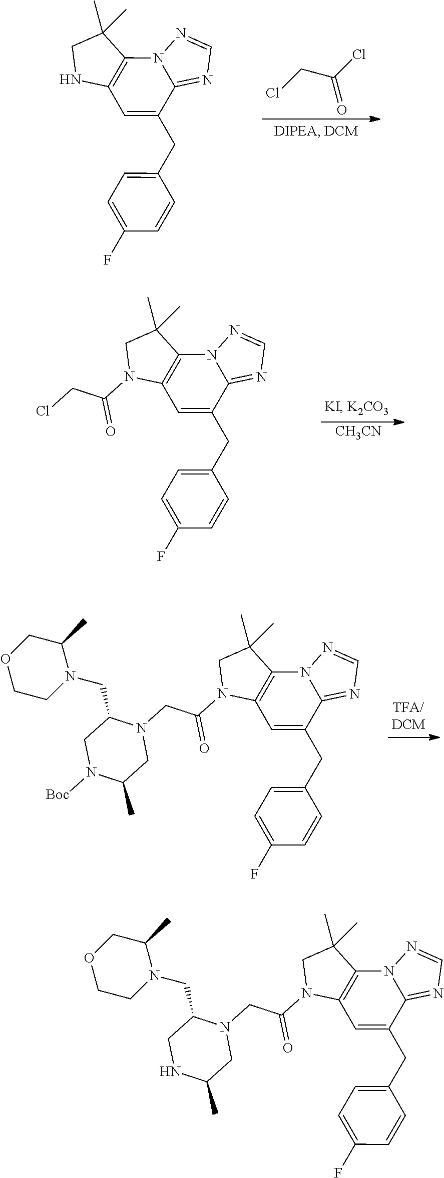 [SMILES: CC1(C)CNc2cc(Cc3ccc(F)cc3)c3ncnn3c21 ; O=C(Cl)CCl ; CC1(C)CN(C(=O)CCl)c2cc(Cc3ccc(F)cc3)c3ncnn3c21 ; "CH3CN" ; C[C@@H]1COCCN1C[C@H]1CN(C(=O)OC(C)(C)C)[C@H](C)CN1CC(=O)N1CC(C)(C)c2c1cc(Cc1ccc(F)cc1)c1ncnn21 ; C[C@@H]1CN(CC(=O)N2CC(C)(C)c3c2cc(Cc2ccc(F)cc2)c2ncnn32)[C@@H](CN2CCOC[C@H]2C)CN1]

Step 1: tert-butyl (1-acetyl-6-(4-fluorobenzyl)-3,3-dimethyl-2,3-dihydro-1H-pyrrolo[3,2-b]pyridin-5-yl)carbamate

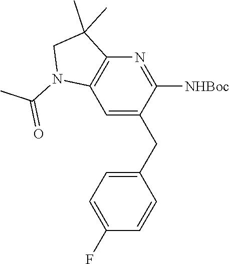 [SMILES: CC(=O)N1CC(C)(C)c2nc(NC(=O)OC(C)(C)C)c(Cc3ccc(F)cc3)cc21]

To a solution of 1-(5-bromo-6-(4-fluorobenzyl)-3,3-dimethyl-2,3-dihydro-1H-pyrrolo[3,2-b]pyridin-1-yl)ethan-1-one (300 mg, 0.795 mmol) in 1,4-Dioxane (10 ml) was added tert-butyl carbamate (112 mg, 0.95 mmol), Cs₂CO₃ (777 mg, 2.39 mmol), dicyclohexyl(2′,4′,6′-triisopropyl-3,6-dimethoxy-[1,1′-biphenyl]-2-yl)phosphane (85 mg, 0.16 mmol) and Pd₂(dba)₃ (72.8 mg, 0.08 mmol). The mixture was filled with N₂ three times and stirred at 90° C. for 2 h. The mixture was cooled to r.t. and diluted with ethyl acetate, washed with H₂O and saturated brine. The organic layer was dried over Na₂SO₄, filtered and concentrated. The crude product was added to a silica gel column and was eluted with ethyl acetate/hexane (ethyl acetate:hexane=7:10) to give tert-butyl (1-acetyl-6-(4-fluorobenzyl)-3,3-dimethyl-2,3-dihydro-1H-pyrrolo[3,2-b]pyridin-5-yl)carbamate (0.19 g, 57.8%) as a white solid. MS (ESI) m/z 414.2 [M+H]⁺.

Step 2: 1-(5-amino-6-(4-fluorobenzyl)-3,3-dimethyl-2,3-dihydro-1H-pyrrolo[3,2-b]pyridin-1-yl)ethan-1-one (Intermediate 2)

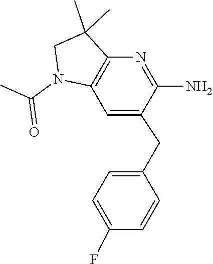 [SMILES: CC(=O)N1CC(C)(C)c2nc(N)c(Cc3ccc(F)cc3)cc21]

To a solution of tert-butyl (1-acetyl-6-(4-fluorobenzyl)-3,3-dimethyl-2,3-dihydro-1H-pyrrolo[3,2-b]pyridin-5-yl)carbamate (0.1 g, 0.24 mmol) in DCM (1 ml) was added TFA (1 ml) at r.t. and the mixture was stirred at r.t. for 1 h. The mixture was concentrated to give crude product 90 mg, which was used in next step without further purification. MS (ESI) m/z 314.2 [M+H]⁺.

Step 3: 1-(4-(4-fluorobenzyl)-8,8-dimethyl-7,8-dihydro-6H-pyrrolo[2,3-e][1,2,4]triazolo[1,5-a]pyridin-6-yl)ethan-1-one

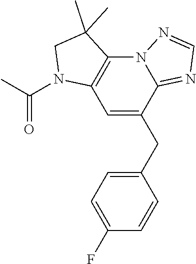 [SMILES: CC(=O)N1CC(C)(C)c2c1cc(Cc1ccc(F)cc1)c1ncnn21]

To a solution of 1-(5-amino-6-(4-fluorobenzyl)-3,3-dimethyl-2,3-dihydro-1H-pyrrolo[3,2-b]pyridin-1-yl)ethan-1-one (90 mg, 0.29 mmol) in i-PrOH (2 ml) was added 1,1-dimethoxy-N,N-dimethylmethanamine (51.3 mg, 0.43 mmol). The mixture was stirred at 80° C. for 2 h. Then hydroxylamine hydrochloride (39.9 mg, 0.57 mmol) was added to the mixture and stirred at 50° C. for 1 h. The mixture was concentrated to give crude product 100 mg, which was used in next step without further purification. To a solution of (E)-N-(1-acetyl-6-(4-fluorobenzyl)-3,3-dimethyl-2,3-dihydro-1H-pyrrolo[3,2-b]pyridin-5-yl)-N′-hydroxyformimidamide (100 mg, 0.281 mmol) in THF (2 ml) was added 2,2,2-trifluoroacetic anhydride (88 mg, 0.42 mmol). The mixture was stirred at r.t. for 2 h. The mixture was concentrated and The crude product was added to a silica gel column and was eluted with methanol/dichloromethane (methanol:dichloromethane=1:20) to give 1-(4-(4-fluorobenzyl)-8,8-dimethyl-7,8-dihydro-6H-pyrrolo[2,3-e][1,2,4]triazolo[1,5-a]pyridin-6-yl)ethan-1-one (50 mg, 52.7%) as a yellow oil. MS (E SI) m/z 339.2 [M+H]⁺.

Step 4: 4-(4-fluorobenzyl)-8,8-dimethyl-7,8-dihydro-6H-pyrrolo[2,3-e][1,2,4]triazolo[1,5-a]pyridine

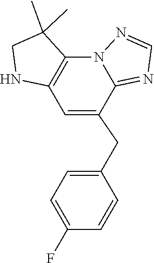 [SMILES: CC1(C)CNc2cc(Cc3ccc(F)cc3)c3ncnn3c21]

To a solution of 1-(4-(4-fluorobenzyl)-8,8-dimethyl-7,8-dihydro-6H-pyrrolo[2,3-e][1,2,4]triazolo[1,5-a]pyridin-6-yl)ethan-1-one (50 mg, 0.15 mmol) in EtOH (2 ml) was added 5M HCl (2 ml). The mixture was stirred at 95° C. for 6 h. The mixture was concentrated and diluted with ethyl acetate, washed with saturated NaHCO₃ (aq) and brine. The organic layer was dried over Na₂SO₄, filtered and concentrated to give crude product 45 mg, which was used next step without further purification. MS (ESI) m/z 297.1 [M+H]⁺.

Step 5: 2-chloro-1-(4-(4-fluorobenzyl)-8,8-dimethyl-7,8-dihydro-6H-pyrrolo[2,3-e][1,2,4]triazolo[1,5-a]pyridin-6-yl)ethan-1-one

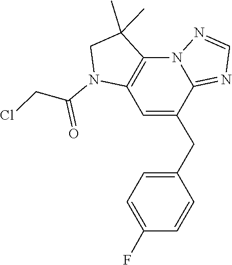 [SMILES: CC1(C)CN(C(=O)CCl)c2cc(Cc3ccc(F)cc3)c3ncnn3c21]

To a solution of 4-(4-fluorobenzyl)-8,8-dimethyl-7,8-dihydro-6H-pyrrolo[2,3-e][1,2,4]triazolo[1,5-a]pyridine (45 mg, 0.15 mmol) in DCM (2 ml) was added N-ethyl-N-isopropylpropan-2-amine (39.3 mg, 0.3 mmol) and 2-chloroacetyl chloride (25.7 mg, 0.23 mmol). The mixture was stirred at r.t. for 1 h. After that the mixture was concentrated to give yellow oil, which was added to a silica gel column and eluted with ethyl acetate/hexane (ethyl acetate:hexane=1:3) to give 2-chloro-1-(4-(4-fluorobenzyl)-8,8-dimethyl-7,8-dihydro-6H-pyrrolo[2,3-e][1,2,4]triazolo[4,3-a]pyridin-6-yl)ethan-1-one (49 mg, 88%) as a yellow oil. MS (ESI) m/z 373.1 [M+H]⁺.

Step 6: tert-butyl (2R,5S)-4-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-m ethylmorpholino)methyl)piperazine-1-carboxylate

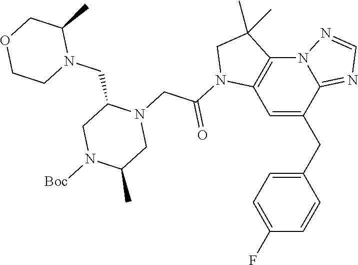 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1ncnn21]

To a solution of 2-chloro-1-(4-(4-fluorobenzyl)-8,8-dimethyl-7,8-dihydro-6H-pyrrolo[2,3-e][1,2,4]triazolo[1,5-a]pyridin-6-yl)ethan-1-one (49 mg, 0.13 mmol) in acetonitrile (2 ml) was added potassium iodide (32.7 mg, 0.2 mmol), K₂CO₃ (54.5 mg, 0.4 mmol) and tert-butyl (2R,5S)-2-methyl-5-(((R)-3-methylmorpholino)methyl)piperazine-1-carboxylate II (41.2 mg, 0.13 mmol). The mixture was stirred at r.t. for 2 h. The mixture was diluted with ethyl acetate, washed with water and brine. The organic layer was dried over Na₂SO₄, filtered and concentrated to give yellow oil, which was added to a silica gel column and eluted with DCM/MeOH (DCM:MeOH=10:1) to afford the title compound (50 mg, 58.5%) as a white solid. MS (ESI) m/z 650.4 [M+H]⁺.

Step 7: 1-(4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl) piperazin-1-yl)ethan-1-one

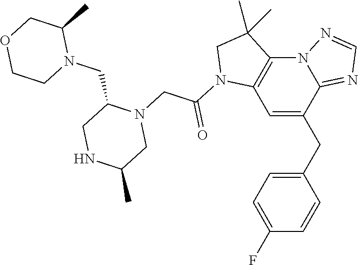 [SMILES: C[C@@H]1CN(CC(=O)N2CC(C)(C)c3c2cc(Cc2ccc(F)cc2)c2ncnn32)[C@@H](CN2CCOC[C@H]2C)CN1]

To a solution of tert-butyl (2R,5S)-4-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (50 mg, 0.08 mmol) in DCM (2 ml) was added TFA (1 ml). The mixture was stirred at r.t. for 1 h. The mixture was concentrated and diluted with ethyl acetate, washed with saturated NaHCO₃ (aq) and brine. The organic layer was dried over Na₂SO₄, filtered and concentrated to give yellow oil, which was added to a Prep HPLC column and eluted with water/acetonitrile (5%-50%) to to give 1-(4-(4-fluorobenz yl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (23.9 mg, 56.5%) as a white solid. ¹H NMR (400 MHz, CD₃OD) δ (ppm) 8.41 (s, 1H), 8.38 (s, 1H), 7.32 (dd, J=8.5, 5.5 Hz, 2H), 7.01 (t, J=8.7 Hz, 2H), 4.41-4.25 (m, 2H), 4.18-3.98 (m, 3H), 3.73-3.52 (m, 3H), 3.43-3.33 (m, 1H), 3.08-2.77 (m, 7H), 2.56-2.41 (m, 2H), 2.33-1.82 (m, 3H), 1.70 (s, 3H), 1.66 (s, 3H), 1.02 (d, J=6.3 Hz, 3H), 0.95 (d, J=6.3 Hz, 3H). MS (ESI) m/z 550.3 [M+H]⁺.

Example 15: N-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)methanesulfonamide (Compound 15)

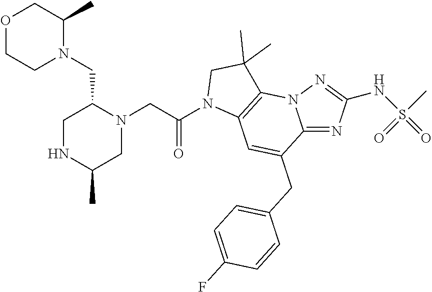 [SMILES: C[C@@H]1CN(CC(=O)N2CC(C)(C)c3c2cc(Cc2ccc(F)cc2)c2nc(NS(C)(=O)=O)nn32)[C@@H](CN2CCOC[C@H]2C)CN1]

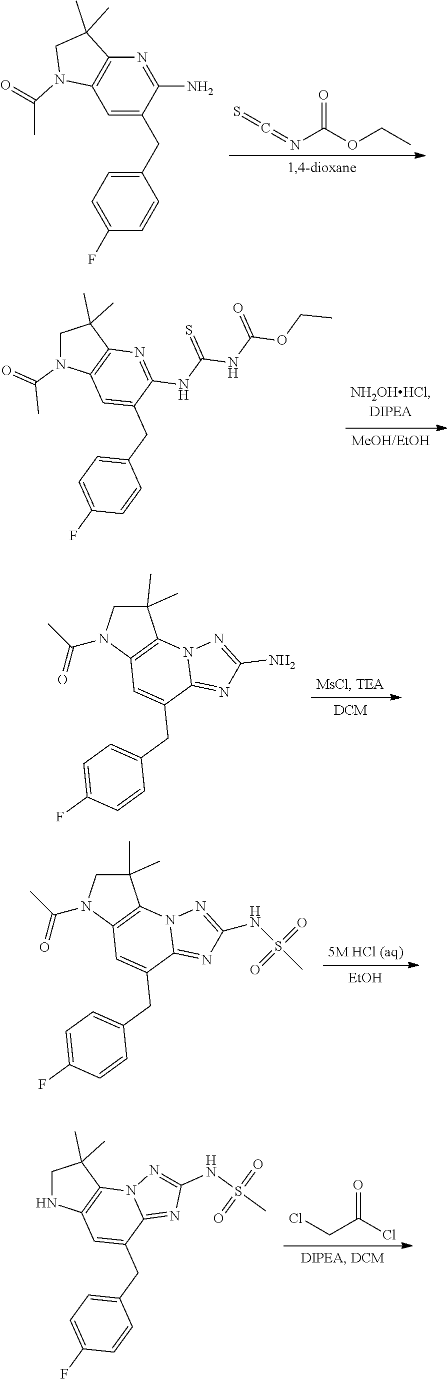

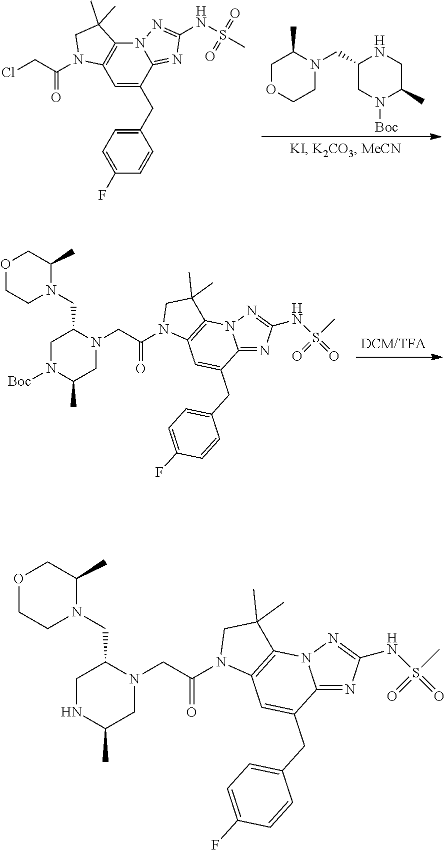

Step 1: 1-(2-amino-4-(4-fluorobenzyl)-8,8-dimethyl-7,8-dihydro-6H-pyrrolo[2,3-e][1,2,4]triazolo[1,5-a]pyridin-6-yl)ethan-1-one

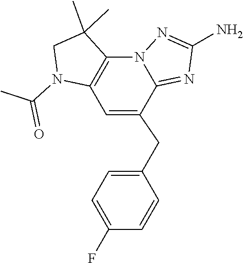 [SMILES: CC(=O)N1CC(C)(C)c2c1cc(Cc1ccc(F)cc1)c1nc(N)nn21]

To a solution of 1-(5-amino-6-(4-fluorobenzyl)-3,3-dimethyl-2,3-dihydro-1H-pyrrolo[3,2-b]pyridin-1-yl)ethan-1-one (200 mg, 0.638 mmol) in 1,4-Dioxane (6 ml) was added O-ethyl carbonisothiocyanatidate (109 mg, 0.83 mmol). The mixture was stirred at r.t. for 1 h. Then the mixture was concentrated and the residue was dissolved in Methanol (6.00 ml)/Ethanol (6.00 ml). After that, hydroxylamine hydrochloride (133 mg, 1.92 mmol) and N-ethyl-N-isopropylpropan-2-a mine (247 mg, 1.92 mmol) was added to the mixture and stirred at 70° C. for 5 h. The mixture was cooled to r.t. and filtrated. The reaction mixture was diluted with dichloromethane and then washed with saturated brine. The organic layer was dried over Na₂SO₄, filtered and concentrated to give yellow oil, which was added to a silica gel column and was eluted with methanol/dichloromethane (methanol:dichloromethane 1:10) to give 1-(2-amino-4-(4-fluorobenzyl)-8,8-dimethyl-7,8-dihydro-6H-pyrrolo[2,3-e][1,2,4]triazolo[1,5-a]pyridin-6-yl)ethan-1-one (100 mg, 44.3%) as a white solid. MS (ESI) m/z 354.2 [M+H]⁺.

Step 2: N-(6-acetyl-4-(4-fluorobenzyl)-8,8-dimethyl-7,8-dihydro-6H-pyrrolo[2,3-e][1,2,4]triazolo[1,5-a]pyridin-2-yl)methanesulfonamide

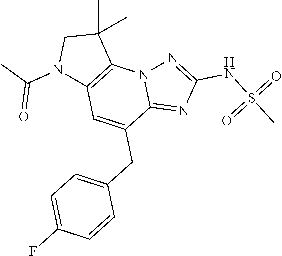 [SMILES: CC(=O)N1CC(C)(C)c2c1cc(Cc1ccc(F)cc1)c1nc(NS(C)(=O)=O)nn21]

To a solution of 1-(2-amino-4-(4-fluorobenzyl)-8,8-dimethyl-7,8-dihydro-6H-pyrrolo[2,3-e][1,2,4]triazolo[1,5-a]pyridin-6-yl)ethan-1-one (100 mg, 0.28 mmol) in DCM (3 ml) was added triethylamine (43.0 mg, 0.42 mmol) and methanesulfonyl chloride (42.1 mg, 0.39 mmol) at 0° C. The mixture was stirred at r.t. for 2 h. After that the mixture was quenched by saturated NaHCO₃ (aq) and diluted with dichloromethane and then washed with H₂O and saturated brine. The organic layer was dried over Na₂SO₄, filtered and concentrated to give yellow oil, which was added to a silica gel column and was eluted with methanol/dichloromethane (methanol:dichloromethane=1:10) to give the title compound (80 mg) as a yellow oil. MS (ESI) m/z 432.1 [M+H]⁺.

Step 3: N-(4-(4-fluorobenzyl)-8,8-dimethyl-7,8-dihydro-6H-pyrrolo[2,3-e][1,2,4]triazolo[1,5-a]pyridin-2-yl)methanesulfonamide

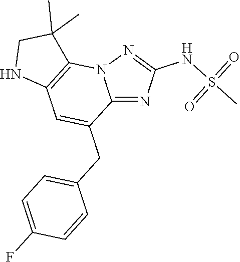 [SMILES: CC1(C)CNc2cc(Cc3ccc(F)cc3)c3nc(NS(C)(=O)=O)nn3c21]

To a solution of a mixture of N-(6-acetyl-4-(4-fluorobenzyl)-8,8-dimethyl-7,8-dihydro-6H-pyrrolo[2,3-e][1,2,4]triazolo[1,5-a]pyridin-2-yl)methanesulfonamide (80 mg) in EtOH (2 ml) was added 5M HCl (2 ml). The mixture was stirred at 95° C. for 6 h. The mixture was concentrated and diluted with ethyl acetate, washed with saturated NaHCO₃ (aq) and brine. The organic layer was dried over Na₂SO₄, filtered and concentrated to give crude title compound, which was used next step without further purification. MS (ESI) m/z 390.1 [M+H]⁺.

Step 4: N-(6-(2-chloroacetyl)-4-(4-fluorobenzyl)-8,8-dimethyl-7,8-dihydro-6H-pyrrolo[2,3-e][1,2,4]triazolo[1,5-a]pyridin-2-yl)methanesulfonamide

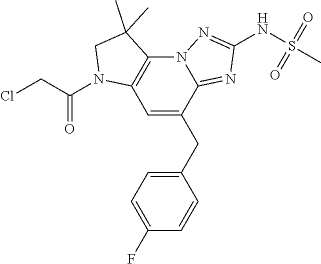 [SMILES: CC1(C)CN(C(=O)CCl)c2cc(Cc3ccc(F)cc3)c3nc(NS(C)(=O)=O)nn3c21]

To a solution of N-(4-(4-fluorobenzyl)-8,8-dimethyl-7,8-dihydro-6H-pyrrolo[2,3-e][1,2,4]triazolo[1,5-a]pyridin-2-yl)methanesulfonamide (40 mg) in DCM (2 ml) was added N-ethyl-N-isopropylpropan-2-amine (66.4 mg, 0.51 mmol) and 2-chloroacetyl chloride (13.9 mg, 0.12 mmol). The mixture was stirred at r.t. for 1 h. After that the mixture was concentrated to give yellow oil, which was added to a silica gel column and eluted with ethyl acetate/hexane (ethyl acetate:hexane=1:3) to give the mixed title compound (41 mg) as a yellow oil. MS (ESI) m/z 466.1 [M+H]⁺.

Step 5: tert-butyl (2R,5S)-4-(2-(4-(4-fluorobenzyl)-8,8-dimethyl-2-(methylsulfonamido)-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

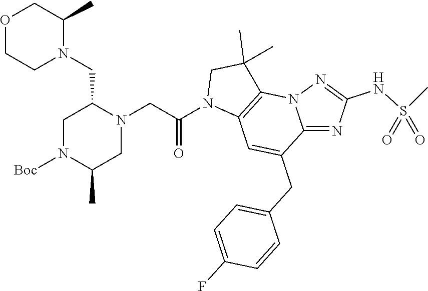 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(NS(C)(=O)=O)nn21]

To a solution of N-(6-(2-chloroacetyl)-4-(4-fluorobenzyl)-8,8-dimethyl-7,8-di hydro-6H-pyrrolo[2,3-e][1,2,4]triazolo[1,5-a]pyridin-2-yl)methanesulfonamide in acetonitrile (2 ml) was added tert-butyl (2R,5S)-2-methyl-5-(((R)-3-methylmorpholino)methyl)piperazine-1-carboxylate II (27.6 mg, 0.09 mmol), K₂CO₃ (36.5 mg, 0.26 mmol) and potassium iodide (21.9 mg, 0.13 mmol). The mixture was stirred at r.t. for 2 h. The mixture was diluted with ethyl acetate, washed with water and saturated brine. The organic layer was dried over Na₂SO₄, filtered and concentrated to give yellow oil, which was added to a silica gel column and eluted with DCM/MeOH (DCM:MeOH=10:1) to afford the title compound (26 mg) as a yellow oil. MS (ESI) m/z 743.3 [M+H]⁺.

Step 6: N-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)methanesulfonamide

To a solution of a mixture of tert-butyl (2R,5S)-4-(2-(4-(4-fluorobenzyl)-8,8-dimethyl-2-(methylsulfonamido)-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate and tert-butyl (2R,5S)-4-(2-(4-(4-fluorobenzyl)-8,8-dimethyl-2-(N-(methylsulfonyl)methylsulfonamido)-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (26 mg) in DCM (2 ml) was added TFA (1 ml). The mixture was stirred at r.t. for 1 h. The mixture was concentrated and diluted with ethyl acetate, washed with saturated NaHCO₃ (aq) and brine. The organic layer was dried over Na₂SO₄, filtered and concentrated to give a yellow oil, which was added to a Prep HPLC column and eluted with water/acetonitrile (5%-50%) to give the title compound (11 mg) as a white solid. ¹H NMR (400 MHz, DMSO-d⁶) δ (ppm) 8.11 (s, 1H), 7.37 (dd, J=8.4, 5.7 Hz, 2H), 7.10 (t, J=8.9 Hz, 2H), 4.22-4.09 (m, 2H), 4.05-3.99 (m, 1H), 3.89-3.66 (m, 2H), 3.55-3.39 (m, 6H), 3.11 (s, 3H), 3.09-2.73 (m, 6H), 2.61-2.51 (m, 2H), 2.22-1.78 (m, 3H), 1.58 (s, 3H), 1.54 (s, 3H), 1.08 (d, J=6.2 Hz, 3H), 0.89 (d, J=6.2 Hz, 3H). MS (ESI) m/z 643.3 [M+H]⁺.

Example 16: N-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)-N-(methylsulfonyl)methanesulfonamide (Compound 16)

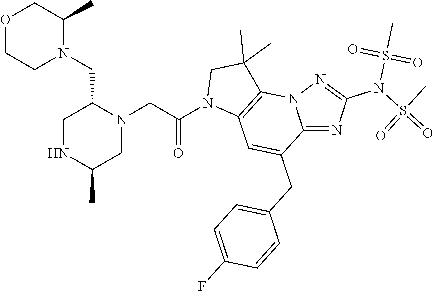 [SMILES: C[C@@H]1CN(CC(=O)N2CC(C)(C)c3c2cc(Cc2ccc(F)cc2)c2nc(N(S(C)(=O)=O)S(C)(=O)=O)nn32)[C@@H](CN2CCOC[C@H]2C)CN1]

Following the synthesis of EXAMPLE 15, EXAMPLE 16 was obtained as a white solid. ¹H NMR (400 MHz, DMSO-d⁶) δ (ppm) 8.19 (s, 1H), 7.35 (dd, J=8.5, 5.7 Hz, 2H), 7.13 (t, J=8.9 Hz, 2H), 4.27-4.15 (m, 2H), 4.07-4.01 (m, 1H), 3.98-3.85 (m, 2H), 3.74-3.45 (m, 4H), 3.27 (s, 3H), 3.22-3.02 (m, 5H), 2.94-2.80 (m, 3H), 2.78-2.57 (m, 2H), 2.30 (s, 3H), 2.21-1.86 (m, 3H), 1.60 (s, 3H), 1.56 (s, 3H), 1.14 (d, J=6.2 Hz, 3H), 0.91 (d, J=6.2 Hz, 3H). MS (ESI) m/z 721.3 [M+H]⁺.

Example 17: 1-(4-(4-fluorobenzyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (Compound 17)

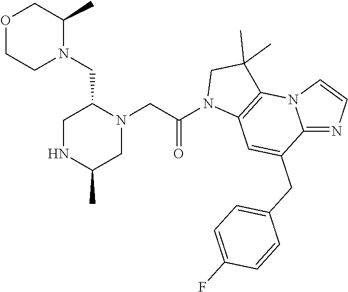 [SMILES: C[C@@H]1CN(CC(=O)N2CC(C)(C)c3c2cc(Cc2ccc(F)cc2)c2nccn32)[C@@H](CN2CCOC[C@H]2C)CN1]

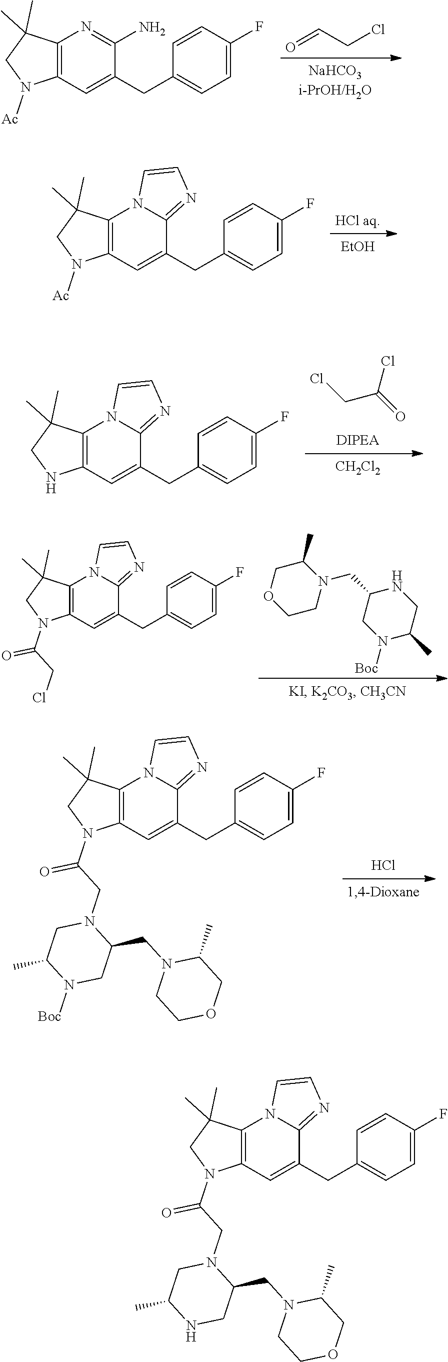

Step 1: 1-(4-(4-fluorobenzyl)-8,8-dimethyl-7,8-dihydro-6H-imidazo[1,2-a]pyrrolo[2,3-e]pyridin-6-yl)ethan-1-one

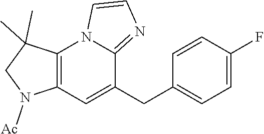 [SMILES: CC(=O)N1CC(C)(C)c2c1cc(Cc1ccc(F)cc1)c1nccn21]

In a flame-dried 100 mL round-bottomed flask, 1-(5-amino-6-(4-fluorobenzyl)-3,3-dimethyl-2,3-dihydro-1H-pyrrolo[3,2-b]pyridin-1-yl)ethan-1-one (115 mg, 0.367 mmol, 1.0 eq) and NaHCO₃ (154 mg, 1.835 mmol, 5.0 eq) were dissolved in 2-propanol (10 mL)/water (1 mL) under nitrogen to give a solution. 2-Chloroacetaldehyde (216 mg, 1.101 mmol, 3.0 eq) was added to the reaction mixture dropwise at 80° C. The reaction mixture was stirred at 80° C. for 16 h, then saturated NaHCO₃ (10 mL) was added to the reaction mixture followed by ex traction with ethyl acetate (15 mL×3). The combined organic layers were dried over Na₂SO₄, filtered and concentrated. The crude product was added to a silica gel column and was eluted with methanol/dichloromethane=1:50 to give 1-(4-(4-fluorobenzyl)-8,8-dimethyl-7,8-dihydro-6H-imidazo[1,2-a]pyrrolo[2,3-e]pyridin-6-yl)ethan-1-one (90 mg, 72.7%) as an off-white solid. MS (ESI) m/z 338.4 [M+H]⁺.

Steps 2-5

Following the synthesis of EXAMPLE 1, steps 2-5 were performed to give 7 mg of EXAMPLE 17 as an off-white solid. ¹H NMR (400 MHz, DMSO-d⁶) δ (ppm) 8.10 (d, J=1.1 Hz, 1H), 8.08 (s, 1H), 7.67 (d, J=1.1 Hz, 1H), 7.40 (dd, J=8.5, 5.7 Hz, 2H), 7.09 (t, J=8.9 Hz, 2H), 4.25 (q, J=14.5 Hz, 2H), 4.08 (d, J=10.6 Hz, 1H), 3.87 (d, J=10.6 Hz, 1H), 3.77 (d, J=16.7 Hz, 1H), 3.63 (d, J=16.2 Hz, 1H), 3.51-3.41 (m, 2H), 3.15 (t, J=9.9 Hz, 1H), 2.99 (s, 1H), 2.94-2.77 (m, 3H), 2.73 (d, J=9.8 Hz, 1H), 2.62 (d, J=8.8 Hz, 2H), 2.28 (d, J=10.9 Hz, 1H), 2.15 (brs, 1H), 1.92 (t, J=9.3 Hz, 1H), 1.75 (d, J=11.4 Hz, 1H), 1.53 (d, J=16.9 Hz, 6H), 0.90 (t, J=5.9 Hz, 6H). MS (ESI) m/z 549.5 [M+H]⁺.

Example 18: ethyl 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-imidazo[1,2-a]pyrrolo[2,3-e]pyridine-2-carboxylate (Compound 18)

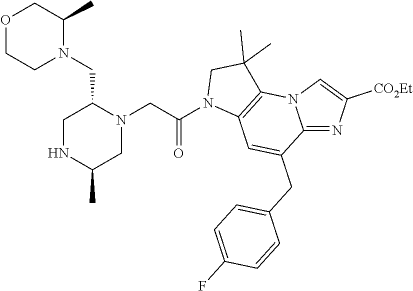 [SMILES: CCOC(=O)c1cn2c3c(cc(Cc4ccc(F)cc4)c2n1)N(C(=O)CN1C[C@@H](C)NC[C@@H]1CN1CCOC[C@H]1C)CC3(C)C]

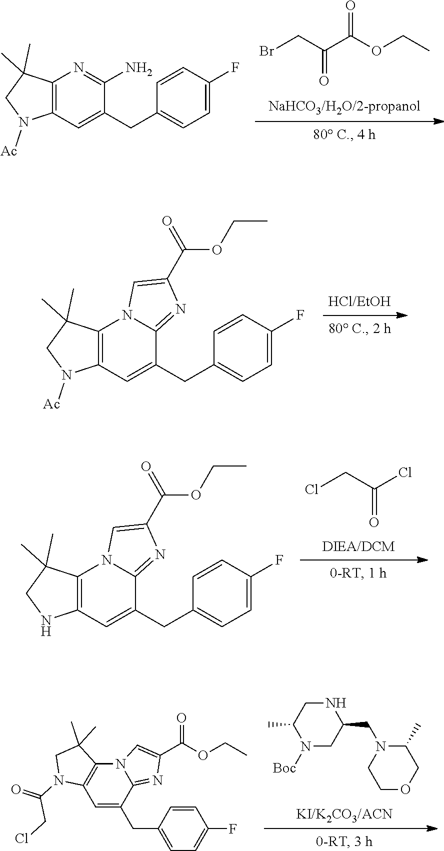

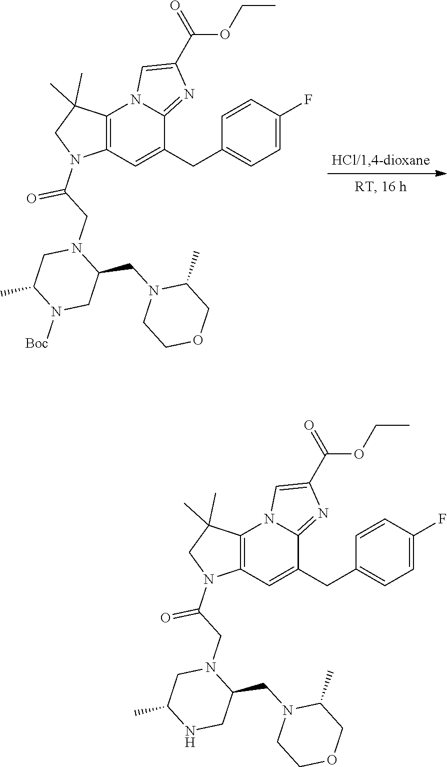 [SMILES: CCOC(=O)c1cn2c3c(cc(Cc4ccc(F)cc4)c2n1)N(C(=O)CN1C[C@@H](C)N(C(=O)OC(C)(C)C)C[C@@H]1CN1CCOC[C@H]1C)CC3(C)C ; "HCl/1,4-dioxane" ; "RT, 16 h" ; CCOC(=O)c1cn2c3c(cc(Cc4ccc(F)cc4)c2n1)N(C(=O)CN1C[C@@H](C)NC[C@@H]1CN1CCOC[C@H]1C)CC3(C)C]

Step 1: ethyl 6-acetyl-4-(4-fluorobenzyl)-8,8-dimethyl-7,8-dihydro-6H-imidazo[1,2-a]pyrrolo[2,3-e]pyridine-2-carboxylate

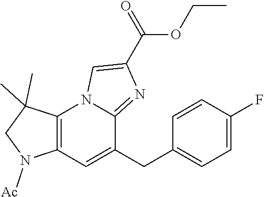 [SMILES: CCOC(=O)c1cn2c3c(cc(Cc4ccc(F)cc4)c2n1)N(C(C)=O)CC3(C)C]

1-(5-amino-6-(4-fluorobenzyl)-3,3-dimethyl-2,3-dihydro-1H-pyrrolo[3,2-b]pyrid in-1-yl)ethan-1-one (0.25 g, 0.798 mmol, 1.0 eq), ethyl 3-bromo-2-oxopropanoate (0.467 g, 2.393 mmol, 3.0 eq), and sodium bicarbonate (0.335 g, 3.99 mmol, 5.0 eq) were dissolved in water (1 ml) and 2-propanol (10.00 ml) under argon to give a yellow solution. The reaction mixture was stirred at 80° C. for 12 h. Saturated brine (20 mL) was added to the reaction mixture followed by extraction with ethyl acetate (40 mL×2). The combined organic layers were dried over Na₂SO₄, filtered and concentrated. The crude product was added to a silica gel column and was eluted with methanol/dichloromethane from 0% to 4% to give the product (0.26 g, 80%) as a yellow oil. MS (ESI) m/z 410.72 [M+H]⁺.

Steps 2-5

Following the synthesis of EXAMPLE 1, steps 2-5 were performed to give 17.4 mg of EXAMPLE 18 as a white solid. ¹H NMR (400 MHz, DMSO-d⁶) δ (ppm) 8.56 (s, 1H), 8.10 (s, 1H), 7.37 (dd, J=8.5, 5.7 Hz, 2H), 7.13 (t, J=8.9 Hz, 2H), 4.39-4.33 (m, 2H), 4.27 (t, J=12.0 Hz, 2H), 4.09 (d, J=10.6 Hz, 1H), 3.87 (d, J=10.6 Hz, 1H), 3.80 (d, J=16.6 Hz, 1H), 3.60 (d, J=16.5 Hz, 1H), 3.51-3.43 (m, 2H), 3.14 (t, J=9.8 Hz, 1H), 2.94 (d, J=9.0 Hz, 1H), 2.85 (ddd, J=26.9, 19.3, 11.6 Hz, 3H), 2.71 (d, J=9.5 Hz, 1H), 2.60 (d, J=8.6 Hz, 2H), 2.43 (t, J=10.9 Hz, 1H), 2.28 (t, J=11.0 Hz, 1H), 2.13 (s, 1H), 1.90 (t, J=9.4 Hz, 1H), 1.74 (d, J=12.0 Hz, 1H), 1.54 (d, J=17.0 Hz, 6H), 1.34 (t, J=7.1 Hz, 3H), 0.88 (t, J=6.7 Hz, 6H). MS (ESI) m/z 621.44 [M+H]⁺.

Example 19: 1-(4-(4-fluorobenzyl)-2-(hydroxymethyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (Compound 19)

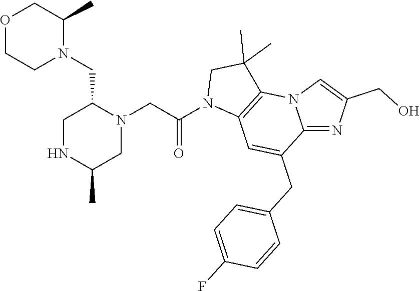 [SMILES: C[C@@H]1CN(CC(=O)N2CC(C)(C)c3c2cc(Cc2ccc(F)cc2)c2nc(CO)cn32)[C@@H](CN2CCOC[C@H]2C)CN1]

EXAMPLE 18 (0.047 g, 0.076 mmol, 1.0 eq) was dissolved in THF (10 ml) under argon to give a solution. The reaction mixture was cooled to 0 T with an ice/water bath. LiBH₄ (3.30 mg, 0.151 mmol, 2.0 eq) was added to the reaction mixture in one portion. The reaction mixture was stirred at rt for 4 h. Saturated NH₄Cl (20 mL) was added to the reaction mixture followed by extraction with ethyl acetate (30 mL×2). The combined organic layers were dried over Na₂SO₄, filtered and concentrated. The crude product was purified by column chromatography to give the product (8.0 mg, 18.26%) as a white solid. ¹H NMR (400 MHz, DMSO-da) δ (ppm) 8.56 (s, 1H), 8.10 (s, 1H), 7.37 (dd, J=8.5, 5.7 Hz, 2H), 7.13 (t, J=8.9 Hz, 2H), 4.39-4.33 (m, 2H), 4.27 (t, J=12.0 Hz, 2H), 4.09 (d, J=10.6 Hz, 1H), 3.87 (d, J=10.6 Hz, 1H), 3.80 (d, J=16.6 Hz, 1H), 3.60 (d, J=16.5 Hz, 1H), 3.51-3.43 (m, 2H), 3.14 (t, J=9.8 Hz, 1H), 2.94 (d, J=9.0 Hz, 1H), 2.85 (ddd, J=26.9, 19.3, 11.6 Hz, 3H), 2.71 (d, J=9.5 Hz, 1H), 2.60 (d, J=8.6 Hz, 2H), 2.43 (t, J=10.9 Hz, 1H), 2.28 (t, J=11.0 Hz, 1H), 2.13 (s, 1H), 1.99 (dd, J=14.7, 7.1 Hz, 1H), 1.90 (t, J=9.4 Hz, 1H), 1.74 (d, J=12.0 Hz, 1H), 1.54 (d, J=17.0 Hz, 6H), 1.34 (t, J=7.1 Hz, 3H), 0.88 (t, J=6.7 Hz, 6H). MS (ESI) m/z 579.40 [M+H]⁺.

Example 20: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-imidazo[1,2-a]pyrrolo[2,3-e]pyridine-2-carboxamide (Compound 20)

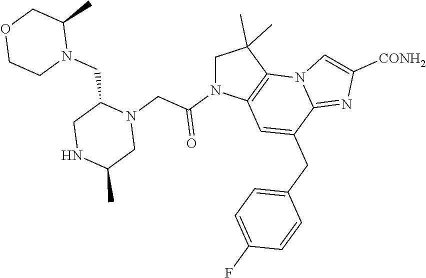 [SMILES: C[C@@H]1CN(CC(=O)N2CC(C)(C)c3c2cc(Cc2ccc(F)cc2)c2nc(C(N)=O)cn32)[C@@H](CN2CCOC[C@H]2C)CN1]

Step 1: Ethyl 6-(2-((2S,5R)-4-(tert-butoxycarbonyl)-5-methyl-2-(((R)-3-methyl morpholino)methyl)piperazin-1-yl)acetyl)-4-(4-fluorobenzyl)-8,8-dimethyl-7,8-dihydro-6H-imidazo[1,2-a]pyrrolo[2,3-e]pyridine-2-carboxylate (0.06 g, 0.083 mmol, 1.0 eq) was dissolved in MeOH (4 ml) and ammonium hydroxide (2 ml) under argon to give a yellow solution. The reaction mixture was stirred at 30° C. for 16 h. Saturated NaCl (30 mL) was added to the reaction mixture followed by extraction with ethyl acetate (40 mL×2). The combined organic layers were dried over Na₂SO₄, filtered and concentrated. The crude product was added to a silica gel column and was eluted with methanol/dichloromethane from 0% to 8% to give the product (0.04 g, 69.5%) as a yellow oil. MS (ESI) m/z 692.58 [M+H]⁺.

Step 2: Tert-butyl (2R,5S)-4-(2-(2-carbamoyl-4-(4-fluorobenzyl)-8,8-dimethyl-7,8-dihydro-6H-imidazo[1,2-a]pyrrolo[2,3-e]pyridin-6-yl)-2-oxoethyl)-2-methyl-5-(((R)-3-methylmorpholino)methyl)piperazine-1-carboxylate (0.04 g, 0.058 mmol, 1.0 eq) was dissolved in DCM (4.00 ml) and TFA (2 ml) under argon to give a yellow solution. The reaction mixture was stirred at rt for 1 h. Saturated NaHCO₃ (30 mL) was added to the reaction mixture followed by extraction with ethyl acetate (40 mL×2). The combined organic layers were dried over Na₂SO₄, filtered and concentrated. The crude product was purified by column chromatography to give the product (13.9 mg, 40.6%) as a white solid. ¹H NMR (400 MHz, DMSO-d⁶) δ (ppm) 8.56 (s, 1H), 8.10 (s, 1H), 7.37 (dd, J=8.5, 5.7 Hz, 2H), 7.13 (t, J=8.9 Hz, 2H), 4.39-4.33 (m, 2H), 4.27 (t, J=12.0 Hz, 2H), 4.09 (d, J=10.6 Hz, 1H), 3.87 (d, J=10.6 Hz, 1H), 3.80 (d, J=16.6 Hz, 1H), 3.60 (d, J=16.5 Hz, 1H), 3.51-3.43 (m, 2H), 3.14 (t, J=9.8 Hz, 1H), 2.94 (d, J=9.0 Hz, 1H), 2.85 (ddd, J=26.9, 19.3, 11.6 Hz, 3H), 2.71 (d, J=9.5 Hz, 1H), 2.60 (d, J=8.6 Hz, 2H), 2.43 (t, J=10.9 Hz, 1H), 2.28 (t, J=11.0 Hz, 1H), 2.13 (s, 1H), 1.90 (t, J=9.4 Hz, 1H), 1.74 (d, J=12.0 Hz, 1H), 1.54 (d, J=17.0 Hz, 6H), 1.34 (t, J=7.1 Hz, 3H), 0.88 (t, J=6.7 Hz, 6H). MS (ESI) m/z 592.42 [M+H]⁺.

Example 21: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-imidazo[1,2-a]pyrrolo[2,3-e]pyridine-2-carbonitrile (Compound 21)

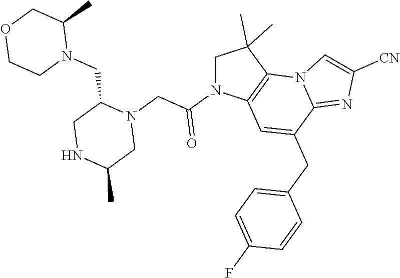 [SMILES: C[C@@H]1CN(CC(=O)N2CC(C)(C)c3c2cc(Cc2ccc(F)cc2)c2nc(C#N)cn32)[C@@H](CN2CCOC[C@H]2C)CN1]

Step 1: Tert-butyl (2R,5S)-4-(2-(2-carbamoyl-4-(4-fluorobenzyl)-8,8-dimethyl-7,8-dihydro-6H-imidazo[1,2-a]pyrrolo[2,3-e]pyridin-6-yl)-2-oxoethyl)-2-methyl-5-(((R)-3-methylmorpholino)methyl)piperazine-1-carboxylate (0.044 g, 0.064 mmol, 1.0 eq), pyridine (0.013 g, 0.159 mmol, 2.5 eq), and TFAA (0.033 g, 0.159 mmol, 2.5 eq) were dissolved in DCM (5 ml) under argon to give a yellow solution. The reaction mixture was stirred at rt for 3 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 8% to give the product (0.034 g, 79%) as a yellow oil. MS (ESI) m/z 674.79 [M+H]⁺.

Step 2: Tert-butyl (2R,5S)-4-(2-(2-cyano-4-(4-fluorobenzyl)-8,8-dimethyl-7,8-dihydro-6H-imidazo[1,2-a]pyrrolo[2,3-e]pyridin-6-yl)-2-oxoethyl)-2-methyl-5-(((R)-3-methylmorpholino)methyl)piperazine-1-carboxylate (0.034 g, 0.050 mmol, 1.0 eq) was dissolved in DCM (4 ml) and TFA (2 ml) under argon to give a yellow solution. The reaction mixture was stirred at rt for 1 h. Saturated NaHCO₃ (30 mL) was added to the reaction mixture followed by extraction with ethyl acetate (40 mL×2). The combined organic layers were dried over Na₂SO₄, filtered and concentrated. The crude product was purified by column chromatography to give the product (13.2 mg, 45.6%) as a white solid. ¹H NMR (400 MHz, DMSO-d⁶) δ (ppm) 8.56 (s, 1H), 8.10 (s, 1H), 7.37 (dd, J=8.5, 5.7 Hz, 2H), 7.13 (t, J=8.9 Hz, 2H), 4.39-4.33 (m, 2H), 4.27 (t, J=12.0 Hz, 2H), 4.09 (d, J=10.6 Hz, 1H), 3.87 (d, J=10.6 Hz, 1H), 3.80 (d, J=16.6 Hz, 1H), 3.60 (d, J=16.5 Hz, 1H), 3.51-3.43 (m, 2H), 3.14 (t, J=9.8 Hz, 1H), 2.94 (d, J=9.0 Hz, 1H), 2.85 (ddd, J=26.9, 19.3, 11.6 Hz, 3H), 2.71 (d, J=9.5 Hz, 1H), 2.60 (d, J=8.6 Hz, 2H), 2.43 (t, J=10.9 Hz, 1H), 2.28 (t, J=11.0 Hz, 1H), 2.13 (s, 1H), 1.99 (dd, J=14.7, 7.1 Hz, 1H), 1.90 (t, J=9.4 Hz, 1H), 1.74 (d, J=12.0 Hz, 1H), 1.54 (d, J=17.0 Hz, 6H), 1.34 (t, J=7.1 Hz, 3H), 0.88 (t, J=6.7 Hz, 6H). MS (ESI) m/z 574.40 [M+H]⁺.

Example 22: 1-(5-(4-fluorobenzyl)-9,9-dimethyl-3-(trifluoromethyl)-8,9-dihydro-7H-pyrrolo[3,2-c][1,2,4]triazolo[4,3-a]pyridin-7-yl)-2-((2R,5R)-5-methyl-2-(((R)-3-methylmorpholino)methyl)piperazin-1-yl)ethan-1-one (Compound 22)

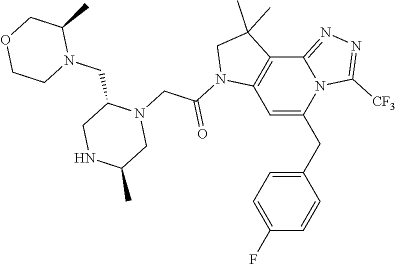 [SMILES: C[C@@H]1CN(CC(=O)N2CC(C)(C)c3c2cc(Cc2ccc(F)cc2)n2c(C(F)(F)F)nnc32)[C@@H](CN2CCOC[C@H]2C)CN1]

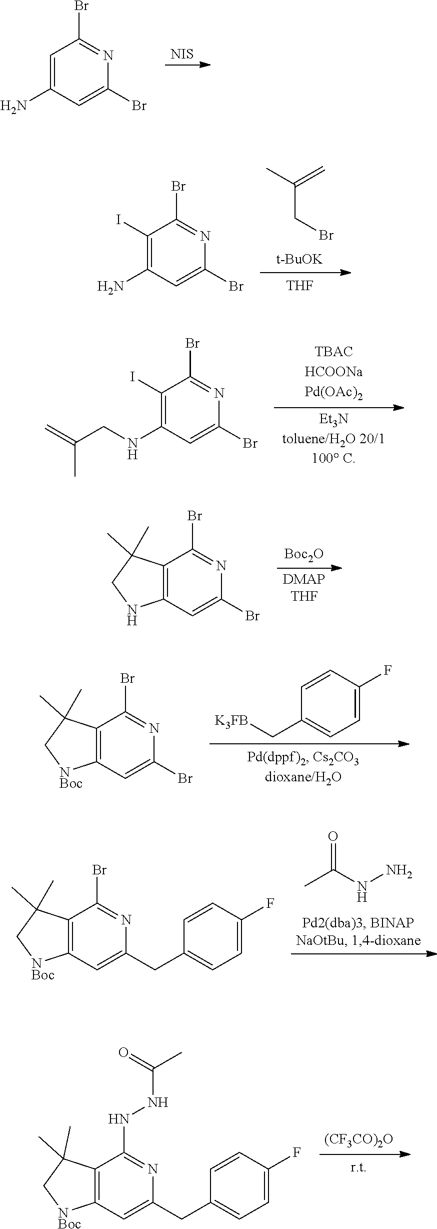

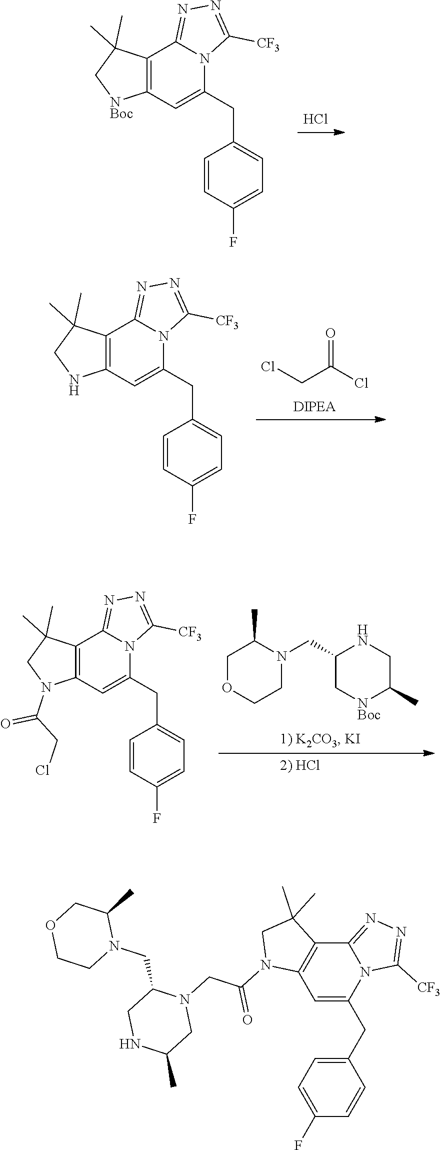

Step 1: 2,6-dibromo-3-iodopyridin-4-amine

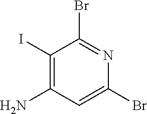

2,6-dibromopyridin-4-amine (4.8 g, 1.0 eq.) was dissolved in DCM (35 mL) and acetic acid (35 mL). Then the mixture was cooled to 0° C., and NIS (4.3 g, 1.0 eq.) were added in portion. The reaction was stirred at R.T. under N₂ for 12 h. TLC indicated the starting material was consumed. The reaction mixture was quenched by addition of aq. NaHCO₃, and then extracted with EA (200 mL×3). The combined organic layers were washed with brine, dried over anhydrous sodium sulfate, filtered and concentrated under reduced pressure. The residue was purified by silica gel column chromatography to afford 2,6-dibromo-3-iodopyridin-4-amine (5.04 g, 70%), LCMS: [M+H]⁺=376/378.

Step 2: 2,6-dibromo-3-iodo-N-(2-methylallyl)pyridin-4-amine

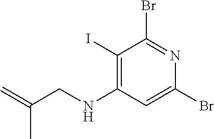

Potassium tert-butoxide (3.3 g, 1.2 eq.) was added to a solution of 2,6-dibromo-3-iodopyridin-4-amine (9.298 g, 1.0 eq.) in dry THF (47 mL) at 0° C. The 3-Bromo-2-methylpropene (3.98 g, 1.2 eq.) was added, while the temperature of the mixture rose to 25° C. The reaction was stirred at R.T. overnight. TLC indicated the starting material was consumed. The reaction mixture was diluted with a saturated aqueous solution of NaHCO₃ and extracted with ethyl acetate. The organic layer was washed with brine and dried over MgSO₄. The residue was purified by silica gel column chromatography to afford 2,6-dibromo-3-iodo-N-(2-methylallyl)pyridin-4-amine (6.36 g, 60%). LCMS: [M+H]⁺=431/433.

Step 3: 4,6-dibromo-3,3-dimethyl-2,3-dihydro-1H-pyrrolo[3,2-c]pyridine

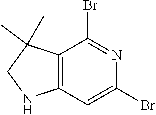

To a stirred solution of 2,6-dibromo-3-iodo-N-(2-methylallyl)pyridin-4-amine (6.6 g. 1.0 eq.) in toluene (132 mL) and H₂O (6.6 mL) was added TBAC (5.11 g, 1.2 eq.), sodium acetate (1.56 g, 1.5 eq.), Pd(OAc)₂ (0.34 g, 0.1 eq.) and TEA (4.64 g, 3.0 eq.). The mixture was purged with nitrogen and heated to 100° C. for 15 h. TLC showed reaction was complete. The reaction mixture was diluted with a saturated aqueous solution of NaHCO₃ and extracted with ethyl acetate. The organic layer was washed with brine and dried over MgSO₄. The residue was purified by silica gel column chromatography to afford desired product (2.3 g, 50%). LCMS: [M+H]⁺=305/307.

Step 4: tert-butyl 4,6-dibromo-3,3-dimethyl-2,3-dihydro-1H-pyrrolo[3,2-c]pyri dine-1-carboxylate

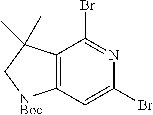 [SMILES: CC(C)(C)OC(=O)N1CC(C)(C)c2c1cc(Br)nc2Br]

To a solution of 4,6-dibromo-3,3-dimethyl-2,3-dihydro-1H-pyrrolo[3,2-c]pyridine (2.3 g, 1.0 eq.) in THF (40 mL) was added Boc₂O (6.58 g, 4.0 eq.) and DMAP (1.38 g, 1.5 eq.). The reaction was stirred at 60° C. for 15 h. TLC showed the starting material was completely consumed. Then the reaction mixture was concentrated under reduced pressure and the result residue was purified by silica gel flash chromatography. The desired product was obtained as a yellow foam (2.754 g, 90%). LCMS: [M+H]⁺=405/407.

Step 5: tert-butyl 4-bromo-6-(4-fluorobenzyl)-3,3-dimethyl-2,3-dihydro-1H-pyrrolo[3,2-c]pyridine-1-carboxylate

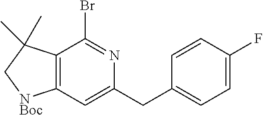 [SMILES: CC(C)(C)OC(=O)N1CC(C)(C)c2c1cc(Cc1ccc(F)cc1)nc2Br]

A vial was charged with tert-butyl 4,6-dibromo-3,3-dimethyl-2,3-dihydro-1H-pyrrolo[3,2-c]pyridine-1-carboxylate (2.1 g, 5.19 mmol), potassium 4-fluorobenz yl-trifluoroborate (1.68 g, 7.78 mmol) (CAS: 1494466-28-2), Pd(dppf)Cl₂ (190 mg, 0.26 mmol), Cs₂CO₃ (6.77 g, 20.8 mmol) and toluene/H₂O (200 mL). The mixture was purged with nitrogen and heated to 75° C. for 12 h. TLC showed reaction was complete. The mixture was diluted with 100 mL H₂O and extracted with ethyl acetate and dried over sodium sulfate. The solvent was removed in vacuum to give desired product (680 mg, 30%). LCMS: [M+H]⁺=435.1.

¹H NMR (400 MHz, DMSO-d⁶) δ (ppm) 7.34-7.27 (m, 2H), 7.18-7.10 (m, 2H), 4.00 (s, 2H), 3.70 (s, 2H), 1.44 (s, 9H), 1.40 (s, 6H).

Step 6: tert-butyl 4-(2-acetylhydrazineyl)-6-(4-fluorobenzyl)-3,3-dimethyl-2,3-dihydro-1H-pyrrolo[3,2-c]pyridine-1-carboxylate

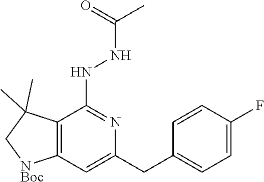 [SMILES: CC(=O)NNc1nc(Cc2ccc(F)cc2)cc2c1C(C)(C)CN2C(=O)OC(C)(C)C]

The mixture of above obtained intermediate (200 mg, 0.46 mmol), acetohydrazide (136 mg, 1.84 mmol), Pd₂(dba)₃ (42.2 mg, 0.046 mmol), BINAP (57.3 mg, 0.092 mmol) and sodium tert-butoxide (88.4 mg, 0.92 mmol) in 1,4-dioxane (10 mL) was stirred at 100° C. for 2 h under nitrogen atmosphere. The react ion mixture was cooled to room temperature, poured into water, and extracted with ethyl acetate. The combined organic phase was washed with water, brine, dried over Na₂SO₄, and concentrated to give the crude product which was purified with silica gel chromatography to obtain the desired product (100 mg, 51%). LCMS: [M+H]⁺=429.26. ¹H NMR (400 MHz, Chloroform-d) δ (ppm) 7.20-7.13 (m, 2H), 6.95 (m, 2H), 6.83 (s, 1H), 3.93 (s, 2H), 3.69 (s, 2H), 1.98 (s, 3H), 1.48 (s, 9H), 1.44 (s, 6H).

Step 7: tert-butyl 5-(4-fluorobenzyl)-9,9-dimethyl-3-(trifluoromethyl)-8,9-dihydro-7H-pyrrolo[3,2-c][1,2,4]triazolo[4,3-a]pyridine-7-carboxylate

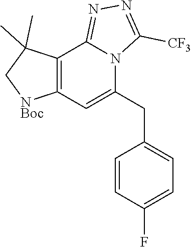 [SMILES: CC(C)(C)OC(=O)N1CC(C)(C)c2c1cc(Cc1ccc(F)cc1)n1c(C(F)(F)F)nnc21]

To a solution of above obtained intermediate (100 mg, 0.234 mmol) in 1,4-dioxane (5 mL) was added trifluoroacetic anhydride (2 mL). The resulting mixture was stirred at rt for 5 h and then concentrated. The crude product was purified with silica gel chromatography to obtain the desired product (30 mg, 26%). LCMS: [M+H]⁺=465.22. ¹H NMR (400 MHz, Chloroform-d) δ (ppm) 7.24-7.17 (m, 2H), 7.11 (m, 2H), 4.34 (s, 2H), 3.85 (s, 2H), 1.65 (s, 9H), 1.59 (s, 6H).

Step 8: 5-(4-fluorobenzyl)-9,9-dimethyl-3-(trifluoromethyl)-8,9-dihydro-7H-pyrrolo[3,2-c][1,2,4]triazolo[4,3-a]pyridine

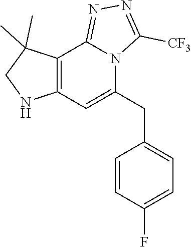 [SMILES: CC1(C)CNc2cc(Cc3ccc(F)cc3)n3c(C(F)(F)F)nnc3c21]

Above obtained compound (30 mg, 0.062 mmol) in hydrogen chloride (4 M solution in dioxane) was stirred for 1 h at 0° C. The solvent was removed to afford the title compound (15 mg, crude), LCMS: [M+H]⁺=365.22.

Steps 9-11: 1-(5-(4-fluorobenzyl)-9,9-dimethyl-3-(trifluoromethyl)-8,9-dihydro-7H-pyrrolo[3,2-c][1,2,4]triazolo[4,3-a]pyridin-7-yl)-2-((2R,5R)-5-methyl-2-(((R)-3-methylmorpholino)methyl)piperazin-1-yl)ethan-1-one

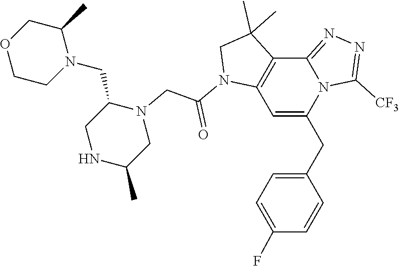 [SMILES: C[C@@H]1CN(CC(=O)N2CC(C)(C)c3c2cc(Cc2ccc(F)cc2)n2c(C(F)(F)F)nnc32)[C@@H](CN2CCOC[C@H]2C)CN1]

To a solution of above obtained compound (15 mg, crude) in DCM was added DIPEA (2.0 eq) and 2-chloroacetyl chloride (1.5 eq) at 0° C. After stirring for 1.5 h, water added to the residue, and the mixture was extracted with DC M. The solvent was removed in vacuum to give a crude product which is directly used in the next step. A mixture of K₂CO₃ (2.0 eq.), KI (1.5 eq.) and tert-butyl (2R,5S)-2-methyl-5-(((R)-3-methylmorpholino)methyl)piperazine-1-carboxylate II (1.2 eq.) was added to a solution of above crude product at 25° C. The reaction was stirred at R.T. for 1 h. LC-MS indicated the starting material was consumed completely and new peak formed. ([M+H]⁺=718.23). The reaction mixture was quenched by addition of water, then extracted with EA, washed with brine, dried (Na₂SO₄) and concentrated under reduced pressure to give a residue which was dissolved in 4M HCl/dioxane. The mixture was stirred at R.T. for 1 h. UPLC-MS indicated a new main peak with desired MS formed, then quenched with water and purified by pre-HPLC (acetonitrile 20% to 50% in 30 min, 45 mL/min). The desired product was obtained as a white solid (3 steps, yield 40%) after lyophilization. LCMS: [M+H]⁺=618.23. ¹H NMR (400 MHz, Methanol-d₄) δ (ppm) 7.50 (s, 1H), 7.43 (dd, J=8.3, 5.2 Hz, 2H), 7.22 (t, J=8.6 Hz, 2H), 4.44 (s, 2H), 4.17 (d, J=10.5 Hz, 1H), 4.07 (d, J=10.0 Hz, 2H), 3.91 (m, 3H), 3.71-3.36 (m, 7H), 3.23-2.87 (m, 5H), 1.69 (d, J=9.0 Hz, 6H), 1.44-1.24 (m, 6H).

Example 23: 1-(5-(4-fluorobenzyl)-9,9-dimethyl-8,9-dihydro-7H-pyrrolo[3,2-c][1,2,4]triazolo[4,3-a]pyridin-7-yl)-2-((2R,5R)-5-methyl-2-(((R)-3-methylmorpholino)methyl)piperazin-1-yl)ethan-1-one (Compound 23)

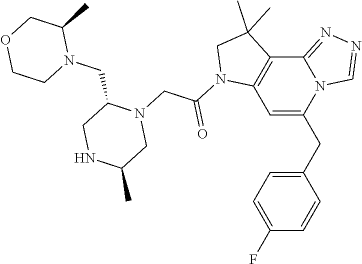 [SMILES: C[C@@H]1CN(CC(=O)N2CC(C)(C)c3c2cc(Cc2ccc(F)cc2)n2cnnc32)[C@@H](CN2CCOC[C@H]2C)CN1]

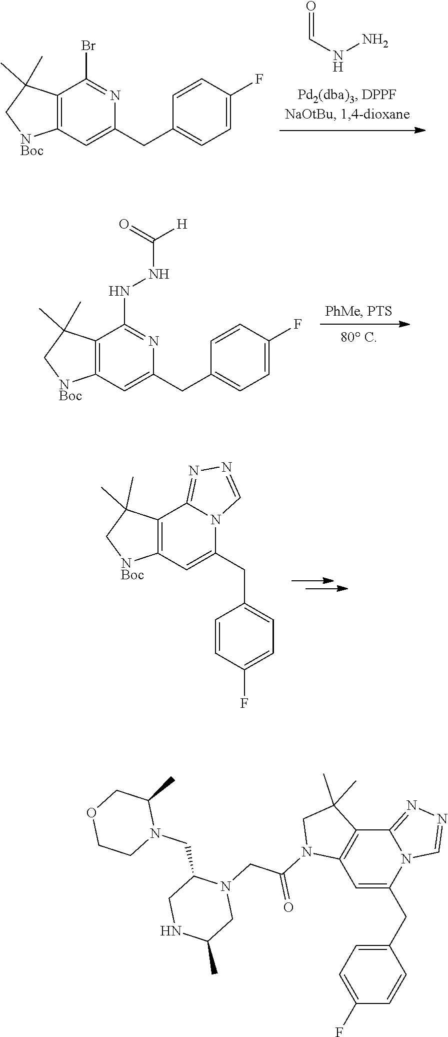

Step 1: tert-butyl 6-(4-fluorobenzyl)-4-(2-formylhydrazineyl)-3,3-dimethyl-2,3-dihydro-1H-pyrrolo[3,2-c]pyridine-1-carboxylate

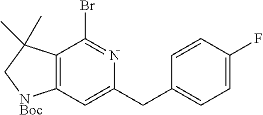 [SMILES: CC(C)(C)OC(=O)N1CC(C)(C)c2c1cc(Cc1ccc(F)cc1)nc2Br]

The mixture of tert-butyl 4-bromo-6-(4-fluorobenzyl)-3,3-dimethyl-2,3-dihydro-1H-pyrrolo[3,2-c]pyridine-1-carboxylate (30 mg, 0.069 mmol), formylhydrazine (16.6 mg, 0.276 mmol), Pd₂(dba)₃ (6.33 mg, 0.0069 mmol), BINAP (7.64 mg, 0.0138 mmol) and sodium tert-butoxide (13.2 mg, 0.138 mmol) in 1,4-dioxane (10 mL) was stirred at 100° C. for 2 h under nitrogen atmosphere. The reaction mixture was cooled to room temperature, poured into water, and extracted with ethyl acetate. The combined organic phase was washed with water, brine, dried over Na₂SO₄, and concentrated to give the crude product which was purified with silica gel chromatography to obtain the desired product (5 mg, 17%). LCMS: [M+H]⁺=415.16.

Step 2: tert-butyl 5-(4-fluorobenzyl)-9,9-dimethyl-8,9-dihydro-7H-pyrrolo[3,2-c][1,2,4]triazolo[4,3-a]pyridine-7-carboxylate

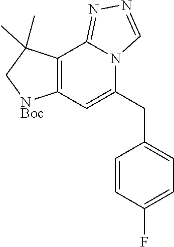 [SMILES: CC(C)(C)OC(=O)N1CC(C)(C)c2c1cc(Cc1ccc(F)cc1)n1cnnc21]

The mixture of above obtained compound (5 mg, 0.012 mmol), p-toluenesulfonic acid (1 mg) in toluene (2 mL) was stirred at 80° C. for 4 h. The react ion mixture was cooled to room temperature and concentrated to give the crude product which was purified with silica gel chromatography to obtain the desi red product (5 mg, 100%). LCMS: [M+H]⁺=397.31.

### Following Steps:

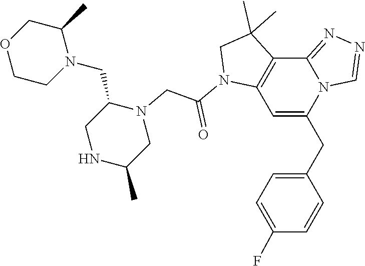 [SMILES: C[C@@H]1CN(CC(=O)N2CC(C)(C)c3c2cc(Cc2ccc(F)cc2)n2cnnc32)[C@@H](CN2CCOC[C@H]2C)CN1]

Following the synthesis of example 22, the desired product was obtained as a white solid after lyophilization. LCMS: [M+H]⁺=550.31. ¹H NMR (400 MHz, Methanol-d₄) δ (ppm) 9.59 (s, 1H), 7.71 (s, 1H), 7.46 (dd, J=8.3, 5.2 Hz, 2H), 7.23 (t, J=8.5 Hz, 2H), 4.53 (s, 2H), 4.30-4.09 (m, 3H), 4.01 (m, 3H), 3.64 (m, 6H), 3.10 (m, 5H), 1.67 (m, 6H), 1.39 (m, 6H).

Example 24: 1-(5-(4-fluorobenzyl)-9,9-dimethyl-8,9-dihydro-7H-imidazo[1,2-a]pyrrolo[3,2-c]pyridin-7-yl)-2-((2R,5R)-5-methyl-2-(((R)-3-methylmorpholino)methyl)piperazin-1-yl)ethan-1-one (Compound 24)

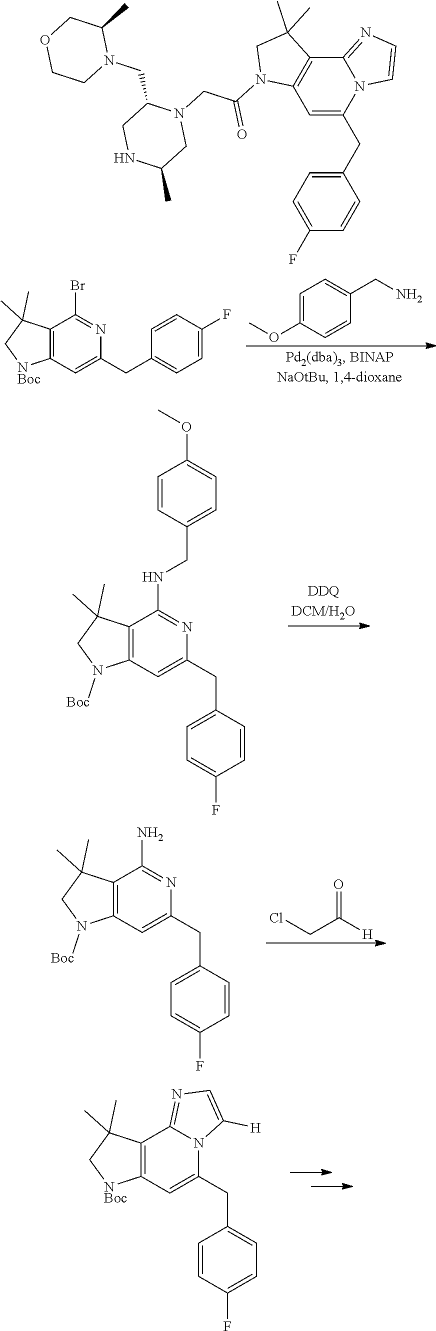

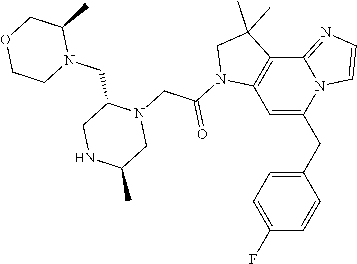 [SMILES: C[C@@H]1CN(CC(=O)N2CC(C)(C)c3c2cc(Cc2ccc(F)cc2)n2ccnc32)[C@@H](CN2CCOC[C@H]2C)CN1]

Step 1: tert-butyl 6-(4-fluorobenzyl)-4-((4-methoxybenzyl)amino)-3,3-dimethyl-2,3-dihydro-1H-pyrrolo[3,2-c]pyridine-1-carboxylate

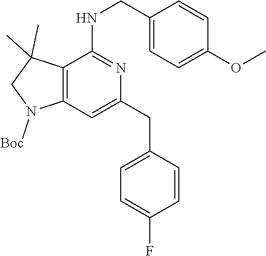

The mixture of tert-butyl 4-bromo-6-(4-fluorobenzyl)-3,3-dimethyl-2,3-dihydro-1H-pyrrolo[3,2-c]pyridine-1-carboxylate (200 mg, 0.46 mmol), 4-methoxybenzylamine (126 mg, 0.92 mmol), Pd₂(dba)₃ (42.2 mg, 0.046 mmol), BINAP (57.3 mg, 0.092 mmol) and sodium tert-butoxide (88.4 mg, 0.92 mmol) in 1,4-dioxane (10 mL) was stirred at 100° C. for 12 h under nitrogen atmosphere. The reaction mixture was cooled to room temperature, poured into water, and extracted with ethyl acetate. The combined organic phase was washed with water, brine, dried over Na₂SO₄, and concentrated to give the crude product which was purified with silica gel chromatography to obtain the desired product (90 mg, 40%). LCMS: [M+H]⁺=492.28. ¹H NMR (400 MHz, DMSO-d⁶) δ (ppm) 7.21-7.11 (m, 4H), 6.96 (t, J=8.7 Hz, 2H), 6.82-6.75 (m, 2H), 6.04 (t, J=6.1 Hz, 1H), 4.50 (d, J=6.0 Hz, 2H), 3.70 (d, J=2.7 Hz, 5H), 3.55 (s, 2H), 1.41 (s, 9H), 1.32 (s, 6H).

Step 2: tert-butyl 4-amino-6-(4-fluorobenzyl)-3,3-dimethyl-2,3-dihydro-1H-pyrrolo[3,2-c]pyridine-1-carboxylate

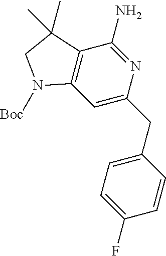 [SMILES: CC(C)(C)OC(=O)N1CC(C)(C)c2c1cc(Cc1ccc(F)cc1)nc2N]

To a solution of above obtained compound (90 mg, 0.183 mmol) in DCM/H₂O (2 mL/0.2 mL) was added DDQ (83.1 mg, 0.366 mmol). The resulting mixture was stirred at rt for 2 h and quenched with saturated NaHCO₃. The layers were separated and the aqueous layer was extracted with DCM (15 mL). The combined organic layers were washed with brine, dried over Na₂SO₄, filtered and concentrated to give the crude product which was purified with silica gel chromatography to obtain the desired product (60 mg, 88%). LCMS: [M+H]=372.32.

Step 3: tert-butyl 5-(4-fluorobenzyl)-9,9-dimethyl-8,9-dihydro-7H-imidazo[1,2-a]pyrrolo[3,2-c]pyridine-7-carboxylate

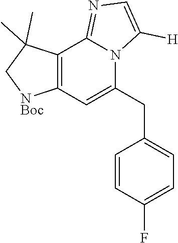 [SMILES: CC(C)(C)OC(=O)N1CC(C)(C)c2c1cc(Cc1ccc(F)cc1)n1ccnc21]

To a mixture of above obtained compound (60 mg, 0.162 mmol) and NaHCO₃ (16.3 mg, 0.194 mmol) in ethanol/H₂O (5 mL/5 mL) was added 3-chloropropionaldehyde (40% aqueous solution) (74.5 mg, 0.324 mmol). The resulting mixture was stirred at 80° C. for 12 h and diluted with H₂O (20 mL). The resulting mixture was extracted with ethyl acetate. The combined organic layers were washed with brine, dried over Na₂SO₄, filtered and concentrated to give the crude product which was purified with silica gel chromatography to obtain the desired product (35 mg, 55%). LCMS: [M+H]⁺=396.30. ¹H NMR (400 MHz, Chloroform-d) δ (ppm) 7.78-7.39 (m, 2H), 7.24-7.13 (m, 2H), 7.10-6.91 (m, 2H), 4.19 (s, 2H), 3.82 (s, 2H), 1.69-1.31 (m, 15H).

### Following Steps:

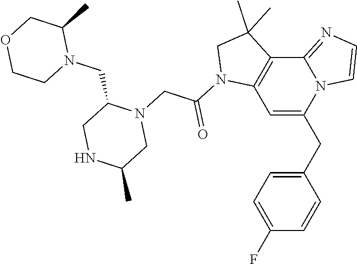 [SMILES: C[C@@H]1CN(CC(=O)N2CC(C)(C)c3c2cc(Cc2ccc(F)cc2)n2ccnc32)[C@@H](CN2CCOC[C@H]2C)CN1]

Following the synthesis of example 22, the desired product example 24 was obtained as a white solid after lyophilization. LCMS: [M+H]⁺=549.3. ¹H NMR (400 MHz, Methanol-d₄) δ (ppm) 8.30 (d, J=2.4 Hz, 1H), 8.09 (d, J=2.4 Hz, 1H), 7.85 (s, 1H), 7.42 (dd, J=8.4, 5.3 Hz, 2H), 7.20 (t, J=8.5 Hz, 2H), 4.49 (s, 2H), 4.26-4.10 (m, 4H), 3.64 (m, 6H), 3.10 (m, 5H), 1.64 (m, 6H), 1.33 (m, 6H).

Example 25: 1-(5-(4-fluorobenzyl)-9,9-dimethyl-8,9-dihydro-7H-pyrrolo[3,2-c][1,2,4]triazolo[1,5-a]pyridin-7-yl)-2-((2R,5R)-5-methyl-2-(((R)-3-methylmorpholino)methyl)piperazin-1-yl)ethan-1-one (Compound 25)

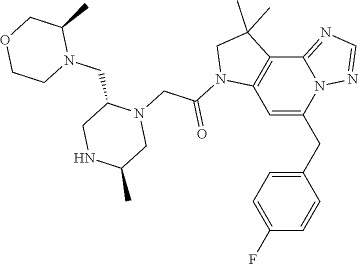 [SMILES: C[C@@H]1CN(CC(=O)N2CC(C)(C)c3c2cc(Cc2ccc(F)cc2)n2ncnc32)[C@@H](CN2CCOC[C@H]2C)CN1]

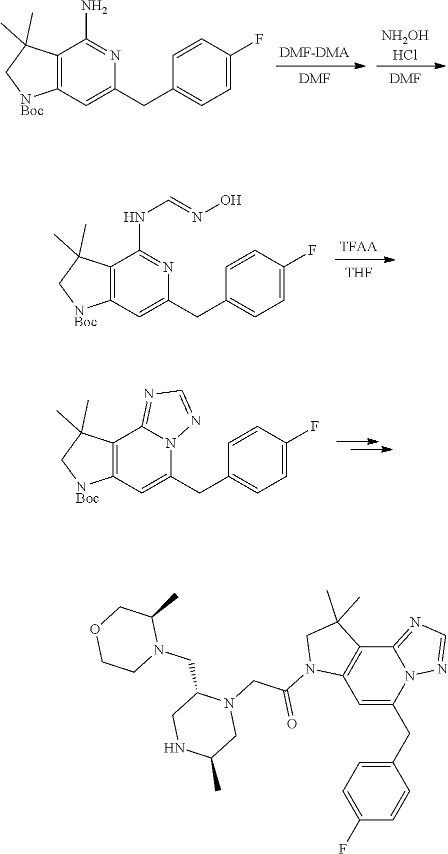

Step 1: tert-butyl (E)-6-(4-fluorobenzyl)-4-(N′-hydroxyformimidamido)-3,3-dimethyl-2,3-dihydro-1H-pyrrolo[3,2-c]pyridine-1-carboxylate

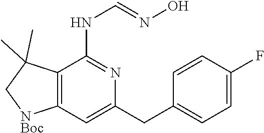 [SMILES: CC(C)(C)OC(=O)N1CC(C)(C)c2c1cc(Cc1ccc(F)cc1)nc2N/C=N/O]

To a stirred solution of tert-butyl 4-amino-6-(4-fluorobenzyl)-3,3-dimethyl-2,3-dihydro-1H-pyrrolo[3,2-c]pyridine-1-carboxylate (50 mg, 0.135 mmol) in DMF (5 mL) was added DMF-DMA (32 mg, 0.27 mmol). The reaction mixture was stirred at 90° C. for 3 h under a nitrogen atmosphere. Upon completion, the reaction was cooled to 50° C. and hydroxylamine hydrochloride (18.6 mg, 0.27 mmol) was added. The mixture was stirred continuously overnight and diluted with H₂O (20 mL). The resulting mixture was extracted with ethyl acetate. The combined organic layers were washed with saturated brine, dried over Na₂S O₄, filtered and concentrated to give the crude product which was purified with silica gel chromatography to obtain the desired product (50 mg, 89%). LC MS: [M+H]⁺=415.30.

Step 2: tert-butyl 5-(4-fluorobenzyl)-9,9-dimethyl-8,9-dihydro-7H-pyrrolo[3,2-c][1,2,4]triazolo[1,5-a]pyridine-7-carboxylate

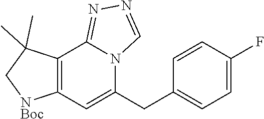 [SMILES: CC(C)(C)OC(=O)N1CC(C)(C)c2c1cc(Cc1ccc(F)cc1)n1cnnc21]

To a solution of above obtained compound (50 mg, 0.121 mmol) in THF (5 mL) was added trifluoroacetic anhydride (0.5 mL). The resulting mixture was stirred at rt for 3 h and then quenched with saturated NaHCO₃. The layers were separated and the aqueous layer was extracted with ethyl acetate (15 mL). The combined organic layers were washed with brine, dried over Na₂SO₄, filtered and concentrated to give the crude product which was purified with silica gel chromatography to obtain the desired product (30 mg, 62%). LCMS: [M+H]⁺=397.30. ¹H NMR (600 MHz, Chloroform-d) δ (ppm) 8.19 (s, 1H), 7.26 (t, J=6.9 Hz, 2H), 7.00-6.88 (m, 2H), 4.35 (s, 2H), 3.77 (s, 2H), 1.55-1.27 (m, 15H).

### Following Steps:

Following the synthesis of example 22, the desired product example 25 was obtained as a white solid after lyophilization. LCMS: [M+H]⁺=550.30. ¹H NMR (400 MHz, Methanol-d₄) δ (ppm) 8.49 (s, 1H), 7.62 (s, 1H), 7.36 (dd, J=8.4, 5.4 Hz, 2H), 7.09 (t, J=8.7 Hz, 2H), 4.48 (d, J=3.2 Hz, 2H), 4.11-3.98 (m, 4H), 3.92 (m, 3H), 3.69-3.39 (m, 6H), 3.18-2.79 (m, 5H), 1.57 (d, J=5.3 Hz, 6H), 1.28 (m, 6H).

Example 26: 1-(5-(4-fluorobenzyl)-9,9-dimethyl-8,9-dihydro-7H-pyrrolo[3,2-f]quinoxalin-7-yl)-2-((2R,5R)-5-methyl-2-(((R)-3-methylmorpholino)methyl) piperazin-1-yl)ethan-1-one (Compound 26)

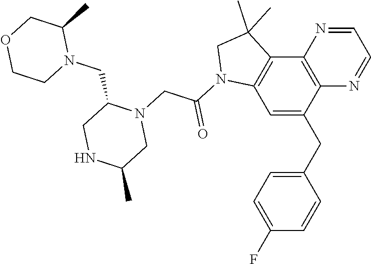 [SMILES: C[C@@H]1CN(CC(=O)N2CC(C)(C)c3c2cc(Cc2ccc(F)cc2)c2nccnc32)[C@@H](CN2CCOC[C@H]2C)CN1]

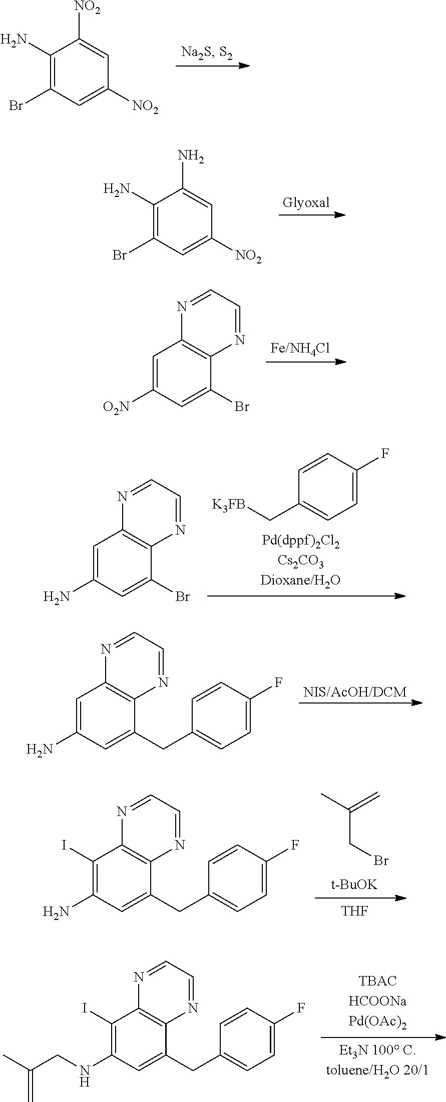

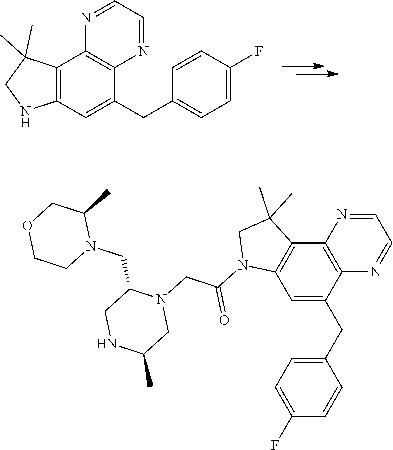 [SMILES: CC1(C)CNc2cc(Cc3ccc(F)cc3)c3nccnc3c21 ; C[C@@H]1CN(CC(=O)N2CC(C)(C)c3c2cc(Cc2ccc(F)cc2)c2nccnc32)[C@@H](CN2CCOC[C@H]2C)CN1]

Step 1: 3-bromo-5-nitrobenzene-1,2-diamine

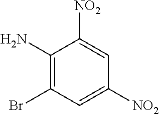 [SMILES: Nc1c(Br)cc([N+](=O)[O-])cc1[N+](=O)[O-]]

To a solution of 2-bromo-4,6-dinitroaniline (60 g, 1.0 eq.) in EtOH (240 mL) and H₂O (60 mL) was added NH₄Cl (12.2 g, 1.0 eq.). Meanwhile, a mixture of Na₂S (55 g, 1.0 eq.) and sulfur powder (7.4 g, 1.0 eq) in EtOH (240 mL) and H₂O (60 mL) was added to the above solution of 2-bromo-4,6-dinitro aniline at 25° C. The mixture was heated at 65° C. for 0.5 h, then NaOH (2 mol/L, 270 mL) was slowly added into the mixture and kept stirred for 30 min. The reaction was allowed to cool and slowly added aq. 2M HCl (270 mL) dropwise. The mixture was partitioned between ethyl acetate and water, the organic extract was dried over Na₂SO₄, concentrated and the residue (LCMS: [M+H]=232/234) was used directly in the next step without further purification.

Step 2: 5-bromo-7-nitroquinoxaline

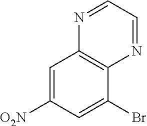 [SMILES: O=[N+]([O-])c1cc(Br)c2nccnc2c1]

To a solution of above obtained intermediate (48 g, 1.0 eq.) in H₂O (1500 mL) was added glyoxal (63.5 g, 1.9 eq., 40% aqueous solution), the mixture was stirred at 100° C. for 4 h. TLC (PE:EA=1:1) indicated the starting material was consumed completely and new spot formed. The reaction was allowed to cool and the solid was filtered to give desired compound as a yellow solid (53 g, crude), LCMS: [M+H]⁺=254/256.

Step 3: 8-bromoquinoxalin-6-amine

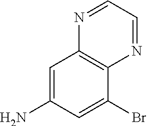 [SMILES: Nc1cc(Br)c2nccnc2c1]

To a solution of above obtained intermediate (15 g, 1.0 eq.) in EtOH (1500 mL) and H₂O (50 mL) was added ammonia solution (31 g, 10 eq). Iron powder (33 g, 10 eq) was added into in portion and the mixture was stirred at 90° C. for 2-3 h and then diluted with H₂O and extracted with ethyl acetate. The organic layer were dried over anhydrous Na₂SO₄, filtered and concentrated under reduced pressure to afford the title compound as a brown solid (6.8 g, 51%). LCMS: [M+H]⁺=224/226.

Step 4: 8-(4-fluorobenzyl)quinoxalin-6-amine

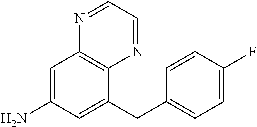 [SMILES: Nc1cc(Cc2ccc(F)cc2)c2nccnc2c1]

Pd(dppf)Cl₂ (1.0 g, 0.1 eq.) was added to a solution of above obtained intermediate (3 g, 1.0 eq.), potassium 4-fluorobenzyl-trifluoroborate (8 g, 2.5 eq) (CAS: 1494466-28-2) and Cs₂CO₃ (10.9 g, 2.5 eq.) in dioxane/H₂O (50 mL/10 mL). The mixture was degassed by evacuating and refilling with nitrogen (×3) then heated at 100° C. for 3 h. The reaction was allowed to cool then partitioned between ethyl acetate and water. The phases were separated then MgSO₄ was added. The solid was filtered then the filtrate was evaporated. The resultant residue was re-dissolved in the minimum of methylene chloride then purified by flash column chromatography on a silica column to give desired compound (1.3 g, 38%), LCMS: [M+H]⁺=254.12.

Step 5: 8-(4-fluorobenzyl)-5-iodoquinoxalin-6-amine

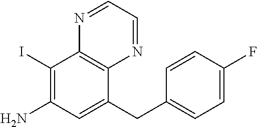 [SMILES: Nc1cc(Cc2ccc(F)cc2)c2nccnc2c1I]

Above obtained compound (1.3 g, 1.0 eq.) was dissolved in DCM (20 mL) and acetic acid (20 mL). Then the mixture was cooled to 0° C., and NIS (1.4 g, 2.2 eq.) were added in portion. The reaction was stirred at R.T. under N₂ for 12 h. TLC indicated the starting material was consumed. The reaction mixture was quenched by addition of aq. NaHCO₃, and then extracted with ethyl acetate. The combined organic layers were washed with brine, dried over anhydrous sodium sulfate, filtered and concentrated under reduced pressure. The residue was purified by silica gel column chromatography to afford desired compound (1.1 g, 55%), LCMS: [M+H]⁺=380.2.

Step 6: 8-(4-fluorobenzyl)-5-iodo-N-(2-methylallyl)quinoxalin-6-amine

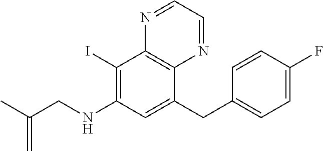 [SMILES: C=C(C)CNc1cc(Cc2ccc(F)cc2)c2nccnc2c1I]

Potassium tert-butoxide (0.68 g, 2.0 eq.) was added to a solution of above obtained intermediate (1.1 g, 1.0 eq.) in dry THF (20 mL) at 0° C. The 3-Bromo-2-methylpropene (0.45 g, 1.1 eq.) was added, while the temperature of the mixture rose to 70° C. The reaction was stirred at 70° C. overnight. TLC indicated the starting material was consumed. The reaction mixture was diluted with a saturated aqueous solution of NaHCO₃ and extracted with ethyl acetate. The organic layer was washed with brine and dried over MgSO₄. The residue was purified by silica gel column chromatography to afford desired compound (0.3 g, 23%), LCMS: [M+H]⁺=434.

Step 7: 5-(4-fluorobenzyl)-9,9-dimethyl-8,9-dihydro-7H-pyrrolo[3,2-f]quinoxaline

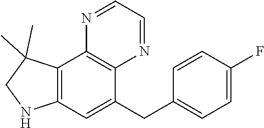 [SMILES: CC1(C)CNc2cc(Cc3ccc(F)cc3)c3nccnc3c21]

To a stirred solution of above obtained compound (0.3 g, 1.0 eq.) in toluene (10 mL) and H₂O (0.5 mL) was added TBAC (0.238 g, 1.2 eq.), sodium acetate (0.073 g, 1.5 eq.), Pd(OAc)₂ (0.016 g, 0.1 eq.) and TEA (0.217 g, 3.0 eq.). The mixture was purged with nitrogen and heated to 100° C. for 15 h. TLC showed reaction was complete. The reaction mixture was diluted with a saturated aqueous solution of NaHCO₃ and extracted with ethyl acetate. The organic layer was washed with brine and dried over MgSO₄. The residue was purified by pre-HPLC to afford desired compound (20 mg, 10%), LCMS: [M+H]⁺=308.2.

### Following Steps:

Following the synthesis of example 22, the desired product example 26 was obtained as a white solid after lyophilization. LCMS: [M+H]⁺=560.2. ¹H NMR (600 MHz, Methanol-d₄) δ (ppm) 8.87 (d, J=1.8 Hz, 1H), 8.83 (d, J=1.8 Hz, 1H), 8.43 (s, 1H), 7.36-7.21 (m, 2H), 7.08-6.98 (m, 2H), 4.69 (d, J=15.8 Hz, 1H), 4.58 (d, J=15.9 Hz, 1H), 4.17-4.03 (m, 3H), 3.69 (m, 2H), 3.62-3.55 (m, 2H), 1.75 (d, J=13.9 Hz, 6H), 1.33-1.28 (m, 6H).

Example 27: 1-(4-(4-fluorobenzyl)-8,8-dimethyl-7,8-dihydro-6H-thiazolo[5,4-e]indol-6-yl)-2-((2R,5R)-5-methyl-2-(((R)-3-methylmorpholino)methyl)piperazin-1-yl)ethan-1-one (Compound 27)

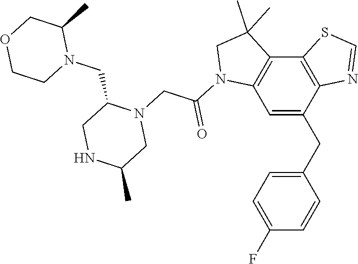 [SMILES: C[C@@H]1CN(CC(=O)N2CC(C)(C)c3c2cc(Cc2ccc(F)cc2)c2ncsc32)[C@@H](CN2CCOC[C@H]2C)CN1]

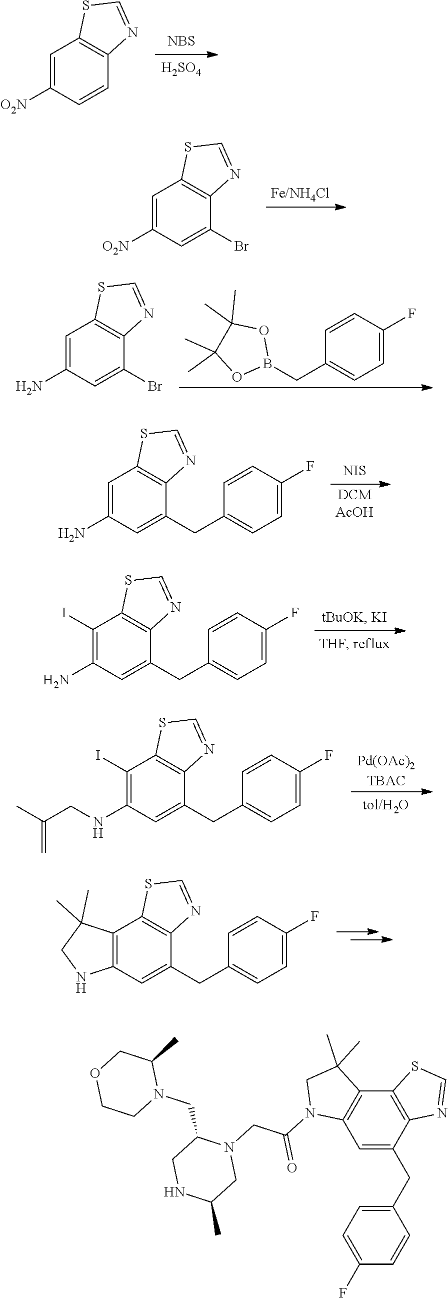

Step 1: 4-bromo-6-nitrobenzo[d]thiazole

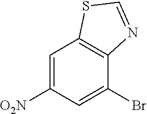 [SMILES: O=[N+]([O-])c1cc(Br)c2ncsc2c1]

To a solution of 6-nitrobenzo[d]thiazole (7.7 g, 1.0 eq.) in con. H₂SO₄ (70 mL) was added NBS (9.2 g, 1.2 eq.) in portionwise at 10° C. The reaction mixture was stirred at 60° C. for 12 h. TLC indicated the starting material was consumed completely and new spot formed. The reaction mixture was slowly poured into ice water, then extracted with ethyl acetate, washed with saturated brine, dried over Na₂SO₄ and concentrated under reduced pressure to give a residue which was purified by silica gel flash chromatography to give desired compound (7 g, 63%), LCMS: [M+H]⁺=259/261.

Step 2: 4-bromobenzo[d]thiazol-6-amine

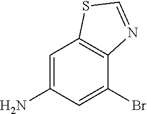 [SMILES: Nc1cc(Br)c2ncsc2c1]

To a solution of above obtained compound (3 g, 1.0 eq.) in EtOH (60 mL) and H₂O (20 mL), was added ammonia solution (6.3 g, 10 eq.). Iron powder (6.5 g, 10 eq.) was added into in portion and the mixture was stirred at 90° C. for 2-3 h and then diluted with H₂O and extracted with ethyl acetate. The organics were dried over anhydrous Na₂SO₄, filtered and concentrated under reduced pressure to afford the title compound as a brown solid (2.4 g, 90%). LC MS: [M+H]⁺=229/231.

Step 3: 4-(4-fluorobenzyl)benzo[d]thiazol-6-amine

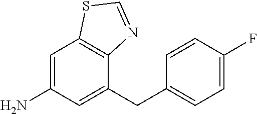 [SMILES: Nc1cc(Cc2ccc(F)cc2)c2ncsc2c1]

Pd(dppf)Cl₂ (0.8 g, 0.1 eq.) was added to a solution of above obtained compound (2.4 g, 1.0 eq.), potassium 4-fluorobenzyl-trifluoroborate (6.3 g, 2.5 eq) (CAS: 1494466-28-2) and Cs₂CO₃ (8.6 g, 2.5 eq.) in dioxane/H₂O (50 mL/10 mL). The mixture was degassed by evacuating and refilling with nitrogen (×3) then heated at 100° C. for 3 h. The reaction was allowed to cool then partitioned between EtOAc and water. The phases were separated then MgSO₄ was added. The solid was filtered then the filtrate was evaporated. The resultant residue was re-dissolved in the minimum of methylene chloride then purified by flash column chromatography on a silica column to give desired compound (1.85 g, 77%), LCMS: [M+H]⁺=259.

Step 4: 4-(4-fluorobenzyl)-7-iodobenzo[d]thiazol-6-amine

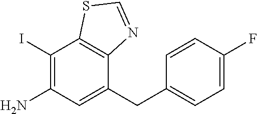 [SMILES: Nc1cc(Cc2ccc(F)cc2)c2ncsc2c1I]

Above obtained compound (1.87 g, 1.0 eq.) was dissolved in DCM (20 mL) and acetic acid (20 mL). Then the mixture was cooled to 0° C., and NIS (1.63 g, 1.0 eq.) were added in portion. The reaction was stirred at R.T. under N₂ for 12 h. TLC indicated the starting material was consumed. The reaction mixture was quenched by addition of aq. NaHCO₃, and then extracted with ethyl acetate. The combined organic layers were washed with brine, dried over anhydrous sodium sulfate, filtered and concentrated under reduced pressure. The residue was purified by silica gel column chromatography to afford desired compound (2.08 g, 74%), LCMS: [M+H]⁺=385.2.

Step 5: 4-(4-fluorobenzyl)-7-iodo-N-(2-methylallyl)benzo[d]thiazol-6-amine

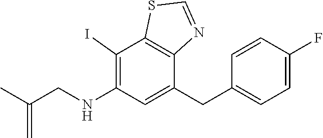 [SMILES: C=C(C)CNc1cc(Cc2ccc(F)cc2)c2ncsc2c1I]

Potassium tert-butoxide (0.234 g, 2.0 eq.) and KI (0.037 g, 0.2 eq.) was added to a solution of above obtained compound (0.4 g, 1.0 eq.) in dry THF (20 mL) at 0° C. The 3-Bromo-2-methylpropene (0.142 g, 1.1 eq.) was added, while the temperature of the mixture rose to 70° C. The reaction was stirred at 70° C. overnight. TLC indicated the starting material was consumed. The reaction mixture was diluted with a saturated aqueous solution of NaHCO₃ and extracted with ethyl acetate. The organic layer was washed with brine and dried over MgSO₄. The residue was purified by silica gel column chromatography to afford the desired compound (0.34 g, 23%), LCMS: [M+H]⁺=439.

Step 6: 4-(4-fluorobenzyl)-8,8-dimethyl-7,8-dihydro-6H-thiazolo[5,4-e]indole

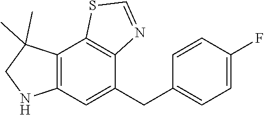 [SMILES: CC1(C)CNc2cc(Cc3ccc(F)cc3)c3ncsc3c21]

To a stirred solution of above obtained compound (0.339 g, 1.0 eq.) in toluene (10 mL) and H₂O (0.5 mL) was added TBAC (0.258 g, 1.2 eq.), sodium acetate (0.079 g, 1.5 eq.), Pd(OAc)₂ (0.018 g, 0.1 eq.) and TEA (0.235 g, 3.0 eq.). The mixture was purged with nitrogen and heated to 100° C. for 15 h. TLC showed reaction was complete. The reaction mixture was diluted with a saturated aqueous solution of NaHCO₃ and extracted with ethyl acetate. The organic layer was washed with brine and dried over MgSO₄. The residue was purified by pre-HPLC to afford the desired compound (20 mg, 16%), LCMS: [M+H]⁺=313.2.

### Following Steps:

Following the synthesis of example 22, the desired product example 27 was obtained as a white solid after lyophilization. LCMS: [M+H]⁺=566.2. ¹H NMR (400 MHz, Methanol-d₄) δ (ppm) 9.23 (s, 1H), 8.16 (s, 1H), 7.38-7.28 (m, 2H), 7.06 (t, J=8.7 Hz, 2H), 4.70-4.44 (m, 2H), 4.09 (m, 6H), 3.68 (m, 3H), 3.45 (m, 3H), 3.24 (m, 2H), 3.08 (m, 2H), 2.21 (t, J=7.6 Hz, 1H), 2.05 (m, 2H), 1.57 (d, J=6.1 Hz, 6H), 1.34-1.30 (m, 6H).

Example 28: 1-(4-(4-fluorobenzyl)-8,8-dimethyl-7,8-dihydro-6H-thiazolo[4,5-e]indol-6-yl)-2-((2R,5R)-5-methyl-2-(((R)-3-methylmorpholino)methyl)piperazin-1-yl)ethan-1-one (Compound 28)

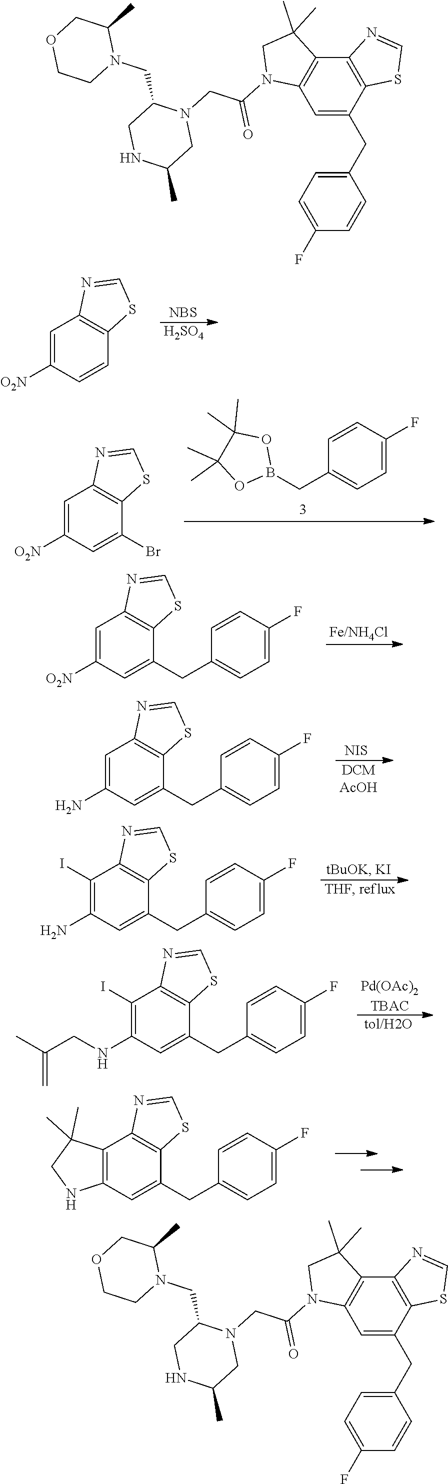

Step 1: 7-bromo-5-nitrobenzo[d]thiazole

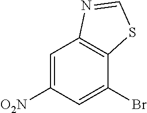 [SMILES: O=[N+]([O-])c1cc(Br)c2scnc2c1]

To a solution of 5-nitrobenzo[d]thiazole (5.0 g, 1.0 eq.) in con. H₂SO₄ (50 mL) was added NBS (7.3 g, 1.2 eq.) in portionwise at 10° C. The reaction mixture was stirred at 60° C. for 12 h. TLC indicated the starting material was consumed completely and new spot formed. The reaction mixture was slowly poured into ice water, then extracted with ethyl acetate, washed with saturated brine, dried over Na₂SO₄ and concentrated under reduced pressure to give a residue which was purified by silica gel flash chromatography to give desired compound (5 g, 70%), LCMS: [M+H]⁺=259/261.

Step 2: 7-(4-fluorobenzyl)-5-nitrobenzo[d]thiazole

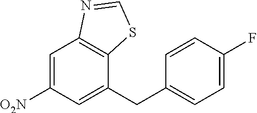 [SMILES: O=[N+]([O-])c1cc(Cc2ccc(F)cc2)c2scnc2c1]

Pd(dppf)Cl₂ (0.82 g, 0.1 eq.) was added to a solution of above obtained compound (2.9 g, 1.0 eq.), potassium 4-fluorobenzyl-trifluoroborate (6.6 g, 2.5 eq) (CAS: 1494466-28-2) and Cs₂CO₃ (9.2 g, 2.5 eq.) in dioxane/H₂O (50 mL/10 mL). The mixture was degassed by evacuating and refing with nitrogen (×3) then heated at 100° C. for 3 h. The reaction was allowed to cool then partitioned between ethyl acetate and water. The phases were separated then MgSO₄ was added. The solid was filtered then the filtrate was evaporated. The resultant residue was re-dissolved in the minimum of methylene chloride then purified by flash column chromatography on a silica column to give desired compound (1.0 g, 30%), LCMS: [M+H]=289.

Step 3: 7-(4-fluorobenzyl)benzo[d]thiazol-5-amine

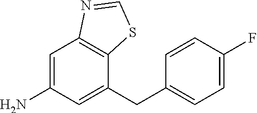 [SMILES: Nc1cc(Cc2ccc(F)cc2)c2scnc2c1]

To a solution of above obtained compound (2 g, 1.0 eq.) in EtOH (60 mL) and H₂O (20 mL), was added ammonia solution (3.8 g, 10 eq.). Iron powder (3.9 g, 10 eq.) was added into in portion and the mixture was stirred at 90° C. for 2-3 h and then diluted with H₂O and extracted with ethyl acetate. The organics were dried over anhydrous Na₂SO₄, filtered and concentrated under reduced pressure to afford the title compound as a brown solid (1.1 g, 62%). LC MS: [M+H]⁺=259.

Step 4: 7-(4-fluorobenzyl)-4-iodobenzo[d]thiazol-5-amine

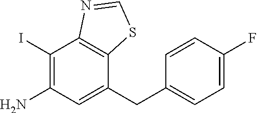 [SMILES: Nc1cc(Cc2ccc(F)cc2)c2scnc2c1I]

Above obtained compound (0.87 g, 1.0 eq.) was dissolved in DCM (10 mL) and acetic acid (10 mL). Then the mixture was cooled to 0° C., and NIS (0.68 g, 1.0 eq.) were added in portion. The reaction was stirred at R.T. under N₂ for 12 h. TLC indicated the starting material was consumed. The reaction mixture was quenched by addition of aq. NaHCO₃, and then extracted with EA. The combined organic layers were washed with brine, dried over anhydrous sodium sulfate, filtered and concentrated under reduced pressure. The residue was purified by silica gel column chromatography to afford desired compound (0.65 g, 56%), LCMS: [M+H]⁺=385.2.

Step 5: 7-(4-fluorobenzyl)-4-iodo-N-(2-methylallyl)benzo[d]thiazol-5-amine

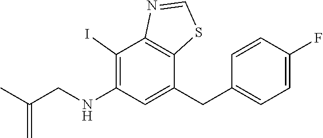 [SMILES: C=C(C)CNc1cc(Cc2ccc(F)cc2)c2scnc2c1I]

Potassium tert-butoxide (0.382 g, 2.0 eq.) and KI (0.057 g, 0.2 eq.) were added to a solution of above obtained compound (0.65 g, 1.0 eq.) in dry THF (20 mL) at 0° C. The 3-Bromo-2-methylpropene (0.276 g, 1.1 eq.) was added, while the temperature of the mixture rose to 70° C. The reaction was stirred at 70° C. overnight. TLC indicated the starting material was consumed. The reaction mixture was diluted with a saturated aqueous solution of NaHCO₃ and extracted with ethyl acetate. The organic layer was washed with brine and dried over MgSO₄. The residue was purified by silica gel column chromatography to afford desired compound (0.1 g, 13%), LCMS: [M+H]⁺=439.

Step 6: 4-(4-fluorobenzyl)-8,8-dimethyl-7,8-dihydro-6H-thiazolo[4,5-e]indole

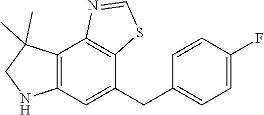 [SMILES: CC1(C)CNc2cc(Cc3ccc(F)cc3)c3scnc3c21]

To a stirred solution of compound 7 (0.168 g, 1.0 eq.) in toluene (10 mL) and H₂O (0.5 mL) was added TBAC (0.128 g, 1.2 eq.), sodium acetate (0.04 g, 1.5 eq.), Pd(OAc)₂ (0.01 g, 0.1 eq.) and TEA (0.117 g, 3.0 eq.). The mixture was purged with nitrogen and heated to 100° C. for 15 h. TLC showed re action was complete. The reaction mixture was diluted with a saturated aqueous solution of NaHCO₃ and extracted with ethyl acetate. The organic layer was washed with brine and dried over MgSO₄. The residue was purified by pre-HP LC to afford desired compound (20 mg, 16%), LCMS: [M+H]⁺=313.2. ¹H NMR (400 MHz, Chloroform-d) δ (ppm) 8.94 (s, 1H), 7.22 (dd, J=8.5, 5.5 Hz, 2H), 7.00 (t, J=8.7 Hz, 2H), 6.64 (s, 1H), 4.11 (s, 2H), 3.45 (s, 2H), 1.61 (s, 6H).

### Following Steps:

Following the synthesis of example 22, the desired product example 28 was obtained as a white solid after lyophilization. LCMS: [M+H]⁺=566.2. ¹H NMR (600 MHz, Chloroform-d) δ (ppm) 9.22 (s, 1H), 7.98 (s, 1H), 7.22 (dd, J=8.3, 5.3 Hz, 2H), 7.04 (t, J=8.4 Hz, 2H), 4.22 (s, 2H), 4.12-2.76 (m, 17H), 2.02 (s, 3H), 1.64 (m, 6H), 1.36 (m, 6H), 1.26 (m, 6H).

Example 29: N-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)acetamide (Compound 29)

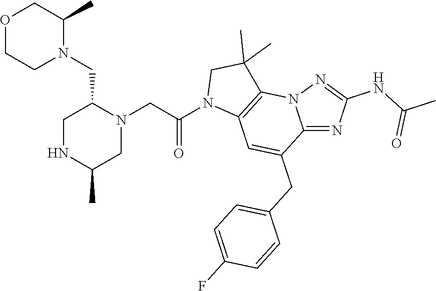 [SMILES: CC(=O)Nc1nc2c(Cc3ccc(F)cc3)cc3c(n2n1)C(C)(C)CN3C(=O)CN1C[C@@H](C)NC[C@@H]1CN1CCOC[C@H]1C]

Compound 29 was synthesized as a white solid (6.0 mg) in a manner similar to that described in compound 15. 1H NMR (400 MHz, DMSO-d6) δ (ppm) 10.79 (s, 1H), 8.31 (s, 1H), 7.37 (dd, J=8.5, 5.7 Hz, 2H), 7.12 (t, J=8.9 Hz, 2H), 4.32-4.09 (m, 3H), 3.97-3.77 (m, 2H), 3.68-3.41 (m, 3H), 3.15 (t, J=10.2 Hz, 1H), 3.00-2.76 (m, 4H), 2.75-2.65 (m, 1H), 2.65-2.55 (m, 2H), 2.48-2.38 (m, 1H), 2.33-2.23 (m, 1H), 2.15 (s, 3H), 1.98-1.86 (m, 1H), 1.80-1.70 (m, 1H), 1.60 (s, 3H), 1.55 (s, 3H), 0.89 (dd, J=12.3, 6.1 Hz, 6H). MS: 607.3 (M+H+).

Example 30: 1-(2-amino-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 30)

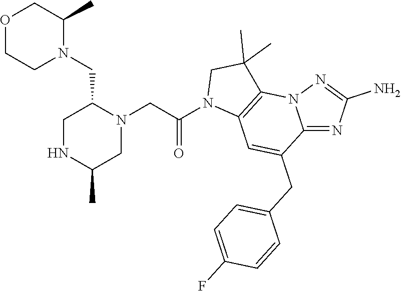 [SMILES: C[C@@H]1CN(CC(=O)N2CC(C)(C)c3c2cc(Cc2ccc(F)cc2)c2nc(N)nn32)[C@@H](CN2CCOC[C@H]2C)CN1]

Compound 30 was synthesized as a white solid (10.0 mg) in a manner similar to that described in compound 15. ¹H NMR (400 MHz, CD₃OD) δ (ppm) 8.29-8.07 (s, 1H), 7.30 (dd, J=8.8, 5.5 Hz, 2H), 7.01 (t, J=8.8 Hz, 2H), 4.24-4.11 (m, 2H), 4.10-3.95 (m, 3H), 3.67-3.52 (m, 3H), 3.41-3.33 (m, 1H), 3.09-2.78 (m, 7H), 2.55-2.44 (m, 2H), 2.31-2.18 (m, 1H), 2.11-1.96 (m, 1H), 1.92-1.79 (m, 1H), 1.66 (s, 3H), 1.62 (s, 3H), 1.04 (d, J=6.3 Hz, 3H), 0.96 (d, J=6.3 Hz, 3H). MS: 565.3 (M+H+).

Example 31: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxamide (Compound 31)

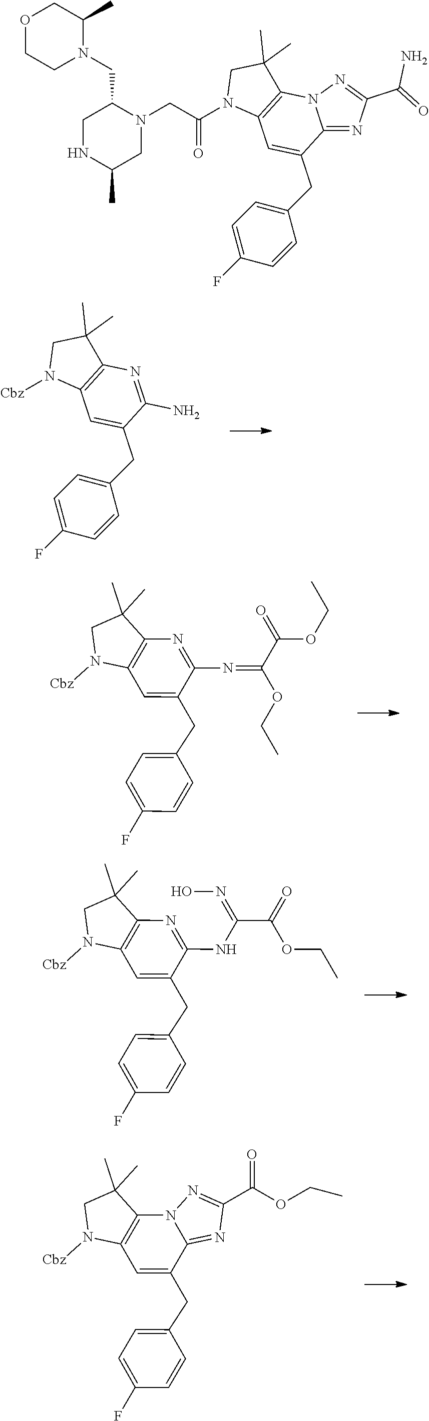

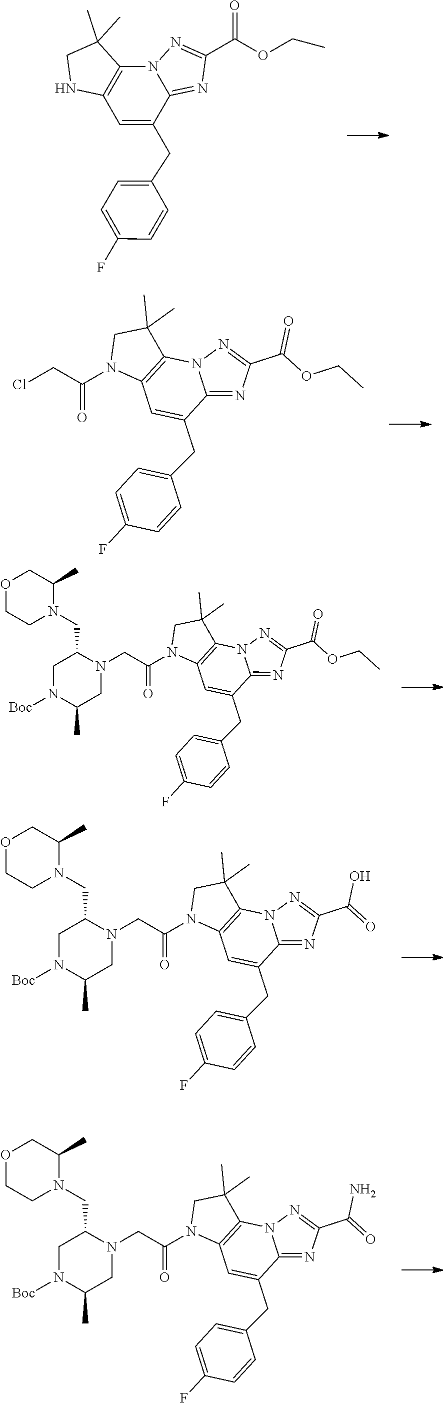

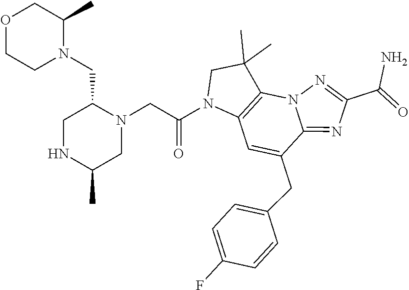 [SMILES: C[C@@H]1CN(CC(=O)N2CC(C)(C)c3c2cc(Cc2ccc(F)cc2)c2nc(C(N)=O)nn32)[C@@H](CN2CCOC[C@H]2C)CN1]

Step 1: benzyl 5-((1,2-diethoxy-2-oxoethylidene)amino)-6-(4-fluorobenzyl)-3,3-dimethyl-2,3-dihydro-1H-pyrrolo[3,2-b]pyridine-1-carboxylate

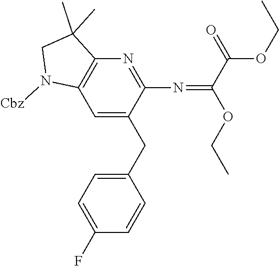 [SMILES: CCOC(=O)C(=Nc1nc2c(cc1Cc1ccc(F)cc1)N(C(=O)OCc1ccccc1)CC2(C)C)OCC]

In a 25 mL vial, benzyl 5-amino-6-(4-fluorobenzyl)-3,3-dimethyl-2,3-dihydro-1H-pyrrolo[3,2-b]pyridine-1-carboxylate (1 g, 2.466 mmol, 1.0 eq), 4-methylbenzenesulfonic acid (0.425 g, 2.466 mmol, 1.0 eq), and ethyl 2,2,2-triethoxyacetate (2.173 g, 9.87 mmol, 4.0 eq) were dissolved in DMF (10 mL). The reaction mixture was heated to 100° C. M.W and stirred for 50 min. The crude product was used for next step directly. MS (ESI) m/z 534.5 [M+H]⁺.

Step 2: benzyl

(Z)-5-(2-ethoxy-N′-hydroxy-2-oxoacetimidamido)-6-(4-fluorobenzyl)-3,3-dimethyl-2,3-dihydro-1H-pyrrolo[3,2-b]pyridine-1-carboxylate

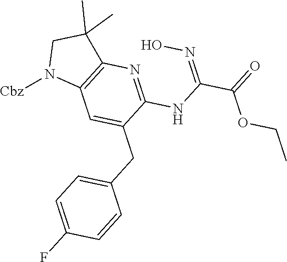 [SMILES: CCOC(=O)/C(=N/O)Nc1nc2c(cc1Cc1ccc(F)cc1)N(C(=O)OCc1ccccc1)CC2(C)C]

In a 25 mL vial, benzyl 5-((1,2-diethoxy-2-oxoethylidene)amino)-6-(4-fluorobenzyl)-3,3-dimethyl-2,3-dihydro-1H-pyrrolo[3,2-b]pyridine-1-carboxylate (1.3 g, 2.436 mmol, 1.0 eq), hydroxylamine hydrochloride (0.846 g, 12.18 mmol, 5.0 eq), and DIEA (1.889 g, 14.62 mmol, 6.0 eq) were dissolved in DMF (10 mL). The reaction mixture was heated to 90° C. M.W and stirred for 50 min. H₂O (20 ml) was added to the reaction mixture followed by extraction with ethyl acetate (20 ml×3). The combined organic layers were washed with brine and dried by Na₂SO₄, filtered and concentrated to give the title compound (1.2 g, 95%) as a light yellow oil. MS (ESI) m/z 522.1 [M+H]⁺.

Step 3: 6-benzyl 2-ethyl 4-(4-fluorobenzyl)-8,8-dimethyl-7,8-dihydro-6H-pyrrolo[2,3-e][1,2,4]triazolo[1,5-a]pyridine-2,6-dicarboxylate

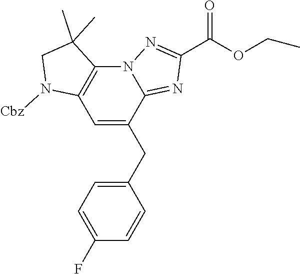 [SMILES: CCOC(=O)c1nc2c(Cc3ccc(F)cc3)cc3c(n2n1)C(C)(C)CN3C(=O)OCc1ccccc1]

In a flame-dried 100 mL round-bottomed flask, benzyl (Z)-5-(2-ethoxy-N′-hydroxy-2-oxoacetimidamido)-6-(4-fluorobenzyl)-3,3-dimethyl-2,3-dihydro-1H-pyrrolo[3,2-b]pyridine-1-carboxylate (1.3 g, 2.497 mmol, 1.0 eq) was dissolved in THF (15 mL). The reaction mixture was cooled to 0° C. with an ice/water bath. TFAA (0.787 g, 3.75 mmol, 1.5 eq) was added to the reaction mixture. The reaction was stirred for 14 h. Saturated NaHCO₃ (15 ml) was added to the reaction mixture followed by extraction with ethyl acetate (20 ml×3). The combined organic layers were dried over Na₂SO₄, filtered and concentrated. The crude product was added to a silica gel column and was eluted with ethyl acetate/hexane from 0-25% to give 6-benzyl 2-ethyl 4-(4-fluorobenzyl)-8,8-dimethyl-7,8-dihydro-6H-pyrrolo[2,3-e][1,2,4]triazolo[1,5-a]pyridine-2,6-dicarboxylate (650 mg, 51.8%) as a white solid. MS (ESI) m/z 504.0 [M+H]⁺.

Step 4: ethyl 4-(4-fluorobenzyl)-8,8-dimethyl-7,8-dihydro-6H-pyrrolo[2,3-e][1,2,4]triazolo[1,5-a]pyridine-2-carboxylate

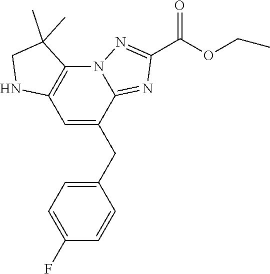 [SMILES: CCOC(=O)c1nc2c(Cc3ccc(F)cc3)cc3c(n2n1)C(C)(C)CN3]

To a solution of 6-benzyl 2-ethyl 4-(4-fluorobenzyl)-8,8-dimethyl-7,8-dihydro-6H-pyrrolo[2,3-e][1,2,4]triazolo[1,5-a]pyridine-2,6-dicarboxylate (80 mg, 0.16 mmol) in THF (10.0 ml) was added Pd/C (10%, 20 mg). The mixture was filled H₂ three times and stirred at room temperature for 1 h. The mixture was filtrated and concentrated. The crude product was added to a silica gel column and was eluted with methanol/dichloromethane (1:20) to give ethyl 4-(4-fluorobenzyl)-8,8-dimethyl-7,8-dihydro-6H-pyrrolo[2,3-e][1,2,4]triazolo[1,5-a]pyridine-2-carboxylate (40 mg, 68.2%) as a colourless oil. MS: 369.2 (M+H+).

Step 5: ethyl 6-(2-chloroacetyl)-4-(4-fluorobenzyl)-8,8-dimethyl-7,8-dihydro-6H-pyrrolo[2,3-e][1,2,4]triazolo[1,5-a]pyridine-2-carboxylate

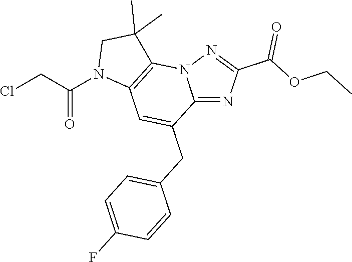 [SMILES: CCOC(=O)c1nc2c(Cc3ccc(F)cc3)cc3c(n2n1)C(C)(C)CN3C(=O)CCl]

To a solution of ethyl 4-(4-fluorobenzyl)-8,8-dimethyl-7,8-dihydro-6H-pyrrolo[2,3-e][1,2,4]triazolo[1,5-a]pyridine-2-carboxylate (40 mg, 0.11 mmol) in DCM (2.0 ml) was added 2-chloroacetyl chloride (14.7 mg, 0.13 mmol). The mixture was stirred at room temperature for 1 h. The mixture was concentrated to give crude product 45 mg, which was used next step directly. MS: 445.1 (M+H⁺).

Step 6: ethyl 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[1,5-a]pyridine-2-carboxylate

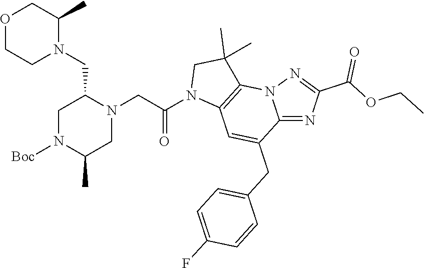 [SMILES: CCOC(=O)c1nc2c(Cc3ccc(F)cc3)cc3c(n2n1)C(C)(C)CN3C(=O)CN1C[C@@H](C)N(C(=O)OC(C)(C)C)C[C@@H]1CN1CCOC[C@H]1C]

To a solution of ethyl 6-(2-chloroacetyl)-4-(4-fluorobenzyl)-8,8-dimethyl-7,8-dihydro-6H-pyrrolo[2,3-e][1,2,4]triazolo[1,5-a]pyridine-2-carboxylate (45 mg, 0.1 mmol) in acetonitrile (3.0 ml) were added tert-butyl (2R,5S)-2-methyl-5-(((R)-3-methylmorpholino)methyl)piperazine-1-carboxylate (31.7 mg, 0.1 mmol), potassium iodide (25.2 mg, 0.15 mmol) and K₂CO₃ (41.9 mg, 0.3 mmol). The mixture was stirred at 29° C. for 2 h. The mixture was filtrated and concentrated to give crude product, which was added to a silica gel column and eluted with DCM/MeOH (10:1) to give the tile compound (60 mg, 82%) as a white solid. MS: 722.4 (M+H⁺).

Step 7: 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[1,5-a]pyridine-2-carboxylic acid

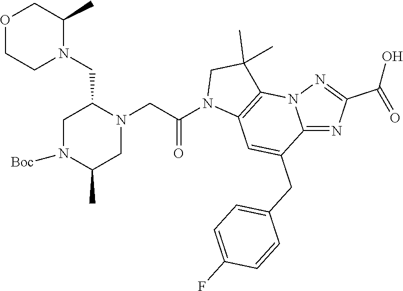 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C(=O)O)nn21]

In a flame-dried 100 mL round-bottomed flask, ethyl 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[1,5-a]pyridine-2-carboxylate (850 mg, 1.177 mmol, 1.0 eq) and LiOH (113 mg, 4.71 mmol, 4.0 eq) were dissolved in THF (2 mL) and water (2 mL). The reaction was stirred for 4 h. The aqueous layer was adjusted to pH 7 with 1N HCl·H₂O (5 mL) was added to the reaction mixture followed by extraction with ethyl acetate (5 mL×3). The combined organic layers were dried over Na₂SO₄, filtered and concentrated to give the tile compound (800 mg, 98%) as a colorless oil. MS (ESI) m/z 594.6 [M+H]⁺.

Step 8: tert-butyl (2R,5S)-4-(2-(2-carbamoyl-4-(4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

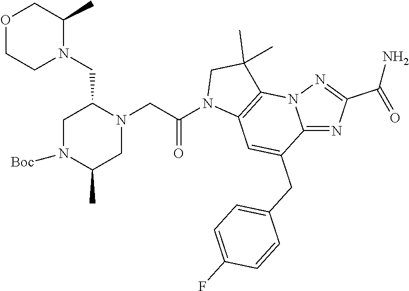 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C(N)=O)nn21]

In a flame-dried 100 mL round-bottomed flask, 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[1,5-a]pyridine-2-carboxylic acid (130 mg, 0.187 mmol, 1.0 eq), DIPEA (97 mg, 0.749 mmol, 4.0 eq), ammonium chloride (20.04 mg, 0.375 mmol, 2.0 eq) and HATU (107 mg, 0.281 mmol, 1.5 eq) were dissolved in DMF (5 mL). The reaction was stirred for 14 h. H₂O (10 ml) was added to the reaction mixture followed by extraction with dichloromethane (5 mL×2). The combined organic layers were dried over Na₂SO₄, filtered and concentrated. The crude product was added to a silica gel column and was eluted with MeOH/DCM from 0˜5% to give the tile compound (130 mg, 100%) as an off-white solid. MS (ESI) m/z 693.5 [M+H]⁺.

Step 9: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxamide (Compound 31)

Compound 31 was obtained as a white solid following similar deprotection conditions. MS (ESI) m/z 593.5 [M+H]⁺. ¹H NMR (400 MHz, DMSO-d⁶) δ (ppm) 8.44 (s, 1H), 8.11 (s, 1H), 7.87 (s, 1H), 7.50-7.42 (m, 2H), 7.15 (dd, J=10.2, 7.6 Hz, 2H), 4.35 (d, J=4.1 Hz, 2H), 4.19 (d, J=10.5 Hz, 1H), 3.98 (d, J=10.6 Hz, 1H), 3.87 (d, J=16.6 Hz, 1H), 3.67 (d, J=16.7 Hz, 1H), 3.49 (dt, J=11.1, 3.3 Hz, 2H), 3.17 (t, J=10.3 Hz, 1H), 3.01-2.80 (m, 3H), 2.73 (dd, J=11.8, 2.8 Hz, 1H), 2.68-2.57 (m, 2H), 2.56-2.53 (m, 2H), 2.46 (t, J=10.9 Hz, 1H), 2.31 (t, J=11.0 Hz, 1H), 2.17 (s, 1H), 1.95 (t, J=10.5 Hz, 1H), 1.78 (d, J=12.4 Hz, 1H), 1.66 (s, 3H), 1.62 (s, 3H), 0.93 (d, J=6.3 Hz, 3H), 0.90 (d, J=5.9 Hz, 3H).

Example 32: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carbonitrile (Compound 32)

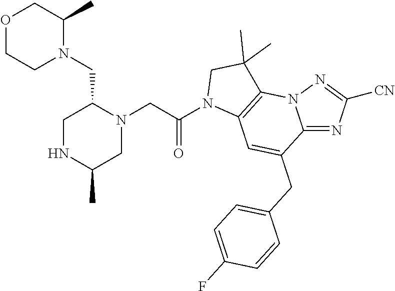 [SMILES: C[C@@H]1CN(CC(=O)N2CC(C)(C)c3c2cc(Cc2ccc(F)cc2)c2nc(C#N)nn32)[C@@H](CN2CCOC[C@H]2C)CN1]

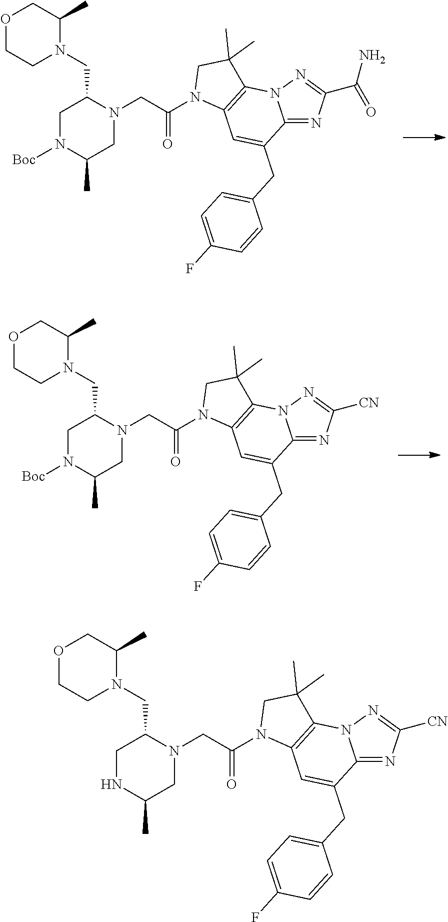 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C(N)=O)nn21 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C#N)nn21 ; C[C@@H]1CN(CC(=O)N2CC(C)(C)c3c2cc(Cc2ccc(F)cc2)c2nc(C#N)nn32)[C@@H](CN2CCOC[C@H]2C)CN1]

Step 1: 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[1,5-a]pyridine-2-carboxylic acid

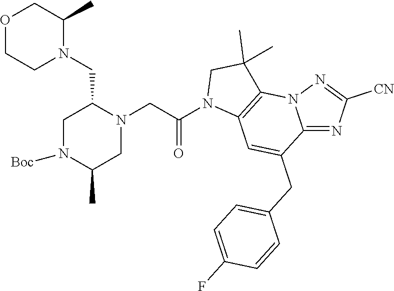 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C#N)nn21]

In a flame-dried 50 mL round-bottomed flask, tert-butyl (2R,5S)-4-(2-(2-carbamoyl-4-(4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (85 mg, 0.123 mmol, 1.0 eq), TFAA (77 mg, 0.368 mmol, 3.0 eq), and pyridine (38.8 mg, 0.491 mmol, 4.0 eq) were dissolved in DCM (5 mL). The result mixture was stirred for 14 h. The reaction mixture was washed with 1M HCl, saturated NaHCO₃, brine, dried over Na₂SO₄, filtered and concentrated. The crude product was added to a silica gel column and was eluted with ethyl acetate/hexane from 0˜40% to give the tile compound (60 mg, 72.5%) as a light yellow solid. MS (ESI) m/z 675.6 [M+H]⁺.

Following Steps: compound 32 was obtained in a manner similar to that described in compound 31. MS (ESI) m/z 575.6 [M+H]⁺.

Example 33: 1-(4-(4-fluorobenzyl)-2,8,8-trimethyl-7,8-dihydro-6H-pyrrolo[2,3-e][1,2,4]triazolo[1,5-a]pyridin-6-yl)-2-((2R,5R)-5-methyl-2-(((R)-3-methyl morpholino)methyl)piperazin-1-yl)ethan-1-one (Compound 36)

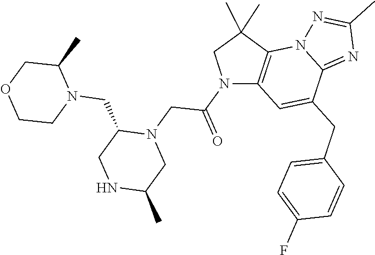 [SMILES: Cc1nc2c(Cc3ccc(F)cc3)cc3c(n2n1)C(C)(C)CN3C(=O)CN1C[C@@H](C)NC[C@@H]1CN1CCOC[C@H]1C]

Compound 36 was synthesized in a manner similar to that described in compound 14 using 1,1-dimethoxy-N,N-dimethylethan-1-amine as the starting material. ¹H NMR (400 MHz, CD₃OD) δ (ppm) 8.31 (s, 1H), 7.31 (dd, J=8.5, 5.4 Hz, 2H), 7.02 (t, J=8.8 Hz, 2H), 4.37-4.21 (m, 2H), 4.13-3.98 (m, 3H), 3.67-3.51 (m, 3H), 3.36 (m, 1H), 3.08-2.78 (m, 7H), 2.59 (s, 3H), 2.48 (t, J=11.3 Hz, 2H), 2.32-2.18 (m, 1H), 2.09-1.97 (m, 1H), 1.90-1.81 (m, 1H), 1.69 (s, 3H), 1.65 (s, 3H), 1.02 (d, J=6.4 Hz, 3H), 0.96 (d, J=6.4 Hz, 3H). MS: 564.3 (M+H⁺).

Example 34: 1-(4-(4-fluorobenzyl)-2-(hydroxymethyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 37)

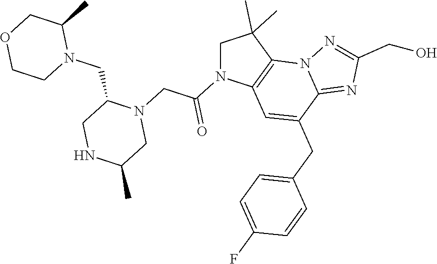 [SMILES: C[C@@H]1CN(CC(=O)N2CC(C)(C)c3c2cc(Cc2ccc(F)cc2)c2nc(CO)nn32)[C@@H](CN2CCOC[C@H]2C)CN1]

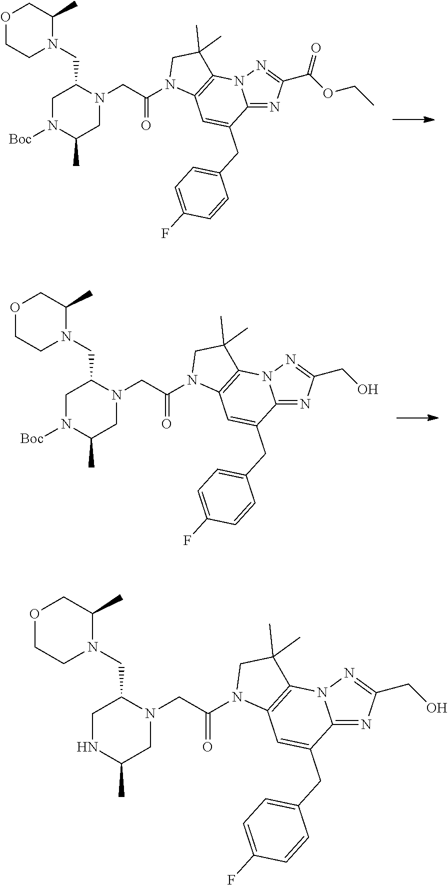 [SMILES: CCOC(=O)c1nc2c(Cc3ccc(F)cc3)cc3c(n2n1)C(C)(C)CN3C(=O)CN1C[C@@H](C)N(C(=O)OC(C)(C)C)C[C@@H]1CN1CCOC[C@H]1C ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(CO)nn21 ; C[C@@H]1CN(CC(=O)N2CC(C)(C)c3c2cc(Cc2ccc(F)cc2)c2nc(CO)nn32)[C@@H](CN2CCOC[C@H]2C)CN1]

Step 1: tert-butyl (2R,5S)-4-(2-(4-(4-fluorobenzyl)-2-(hydroxymethyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methyl morpholino)methyl)piperazine-1-carboxylate

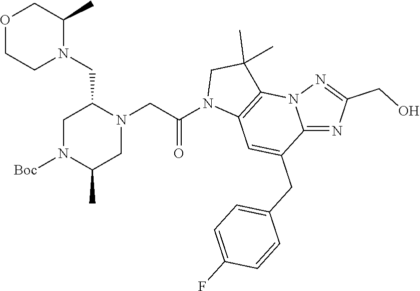 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(CO)nn21]

To a solution of ethyl 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[1,5-a]pyridine-2-carboxylate (50 mg, 0.069 mmol) in THF (2.0 ml)/EtOH (2.0 ml) was added LiBH₄ (9.1 mg, 0.42 mmol). The mixture was stirred at room temperature for 1 h. After concentration, the crude product was added to a silica gel column and eluted with methanol/dichloromethane (1:10) to give the tile compound (26 mg, 55.2%) as a white solid. MS: 680.5 (M+H⁺).

Step 2: Compound 37 was obtained (8 mg, 36.1%) as a white solid using similar deprotection conditions. ¹H NMR (400 MHz, CD₃OD) δ (ppm) 8.35 (s, 1H), 7.33 (dd, J=8.5, 5.5 Hz, 2H), 7.02 (t, J=8.8 Hz, 2H), 4.86 (s, 2H), 4.42-4.25 (m, 2H), 4.16-4.00 (m, 3H), 3.70-3.52 (m, 3H), 3.39-3.33 (m, 1H), 3.11-2.78 (m, 7H), 2.58-2.43 (m, 2H), 2.33-2.18 (m, 1H), 2.09-1.96 (m, 1H), 1.92-1.80 (m, 1H), 1.71 (s, 3H), 1.67 (s, 3H), 1.03 (d, J=6.4 Hz, 3H), 0.96 (d, J=6.4 Hz, 3H). MS: 580.3 (M+H⁺).

Example 35: 1-(4-(2-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino) methyl)piperazin-1-yl)ethan-1-one (Compound 38)

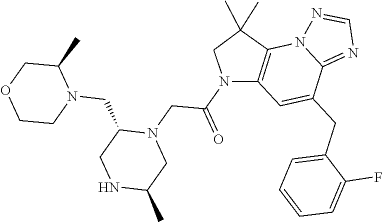 [SMILES: C[C@@H]1CN(CC(=O)N2CC(C)(C)c3c2cc(Cc2ccccc2F)c2ncnn32)[C@@H](CN2CCOC[C@H]2C)CN1]

Compound 38 was synthesized in a manner similar to that described in compound 14. MS (ESI) m/z 550.6[M+H]⁺. ¹H NMR (400 MHz, DMSO-d⁶) δ (ppm) 8.55 (s, 1H), 8.35 (s, 1H), 7.50-7.40 (m, 1H), 7.38-7.27 (m, 1H), 7.27-7.10 (m, 2H), 4.45-4.26 (m, 2H), 4.18 (d, J=10.6 Hz, 1H), 3.96 (d, J=10.6 Hz, 1H), 3.81 (d, J=16.7 Hz, 1H), 3.68 (d, J=16.6 Hz, 1H), 3.54-3.44 (m, 2H), 3.19-3.10 (m, 1H), 3.06-2.95 (m, 1H), 2.94-2.78 (m, 3H), 2.75-2.67 (m, 1H), 2.65-2.57 (m, 2H), 2.51-2.43 (m, 1H), 2.35-2.25 (m, 1H), 2.22-2.11 (m, 1H), 1.98-1.87 (m, 1H), 1.77 (d, J=12.3 Hz, 1H), 1.62 (d, J=18.4 Hz, 6H), 1.01-0.85 (m, 6H).

Example 36: 1-(4-(3-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 39)

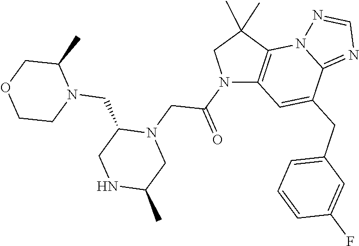 [SMILES: C[C@@H]1CN(CC(=O)N2CC(C)(C)c3c2cc(Cc2cccc(F)c2)c2ncnn32)[C@@H](CN2CCOC[C@H]2C)CN1]

Compound 39 was synthesized in a manner similar to that described in compound 14. ¹H NMR (400 MHz, DMSO-d⁶) δ (ppm) 8.51 (s, 1H), 8.41 (s, 1H), 7.38-7.27 (m, 1H), 7.22-7.16 (m, 2H), 7.09-6.97 (m, 1H), 4.38-4.27 (m, 2H), 4.15 (d, J=10.6 Hz, 1H), 3.97-3.91 (m, 1H), 3.81 (d, J=16.6 Hz, 1H), 3.65 (d, J=16.6 Hz, 1H), 3.49-3.42 (m, 2H), 3.21-3.09 (m, 1H), 3.00-2.93 (m, 1H), 2.91-2.78 (m, 3H), 2.75-2.65 (m, 1H), 2.64-2.55 (m, 2H), 2.46-2.38 (m, 1H), 2.33-2.21 (m, 1H), 2.21-2.08 (m, 1H), 1.95-1.86 (m, 1H), 1.75 (d, J=12.0 Hz, 1H), 1.59 (d, J=16.9 Hz, 6H), 0.91-0.80 (m, 6H).

Example 37: 1-(4-(2,4-difluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 40)

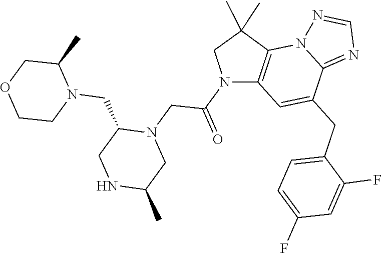 [SMILES: C[C@@H]1CN(CC(=O)N2CC(C)(C)c3c2cc(Cc2ccc(F)cc2F)c2ncnn32)[C@@H](CN2CCOC[C@H]2C)CN1]

Compound 40 was synthesized in a manner similar to that described in compound 14. MS (ESI) m/z 568.5[M+H]⁺. ¹H NMR (400 MHz, DMSO-d⁶) δ (ppm) 8.55 (s, 1H), 8.35 (s, 1H), 7.56-7.43 (m, 1H), 7.34-7.20 (m, 1H), 7.16-7.01 (m, 1H), 4.45-4.25 (m, 2H), 4.18 (d, J=10.6 Hz, 1H), 3.97 (d, J=10.6 Hz, 1H), 3.83 (d, J=16.7 Hz, 1H), 3.68 (d, J=16.7 Hz, 1H), 3.49 (d, J=11.1 Hz, 2H), 3.23-3.09 (m, 1H), 3.05-2.95 (m, 1H), 2.93-2.78 (m, 3H), 2.76-2.68 (m, 1H), 2.66-2.56 (m, 2H), 2.51-2.42 (m, 1H), 2.36-2.25 (m, 1H), 2.23-2.09 (m, 1H), 2.01-1.88 (m, 1H), 1.82-1.71 (m, 1H), 1.62 (d, J=18.0 Hz, 6H), 0.98-0.84 (m, 6H).

Example 38: 1-(4-(4-fluoro-2-methoxybenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 41)

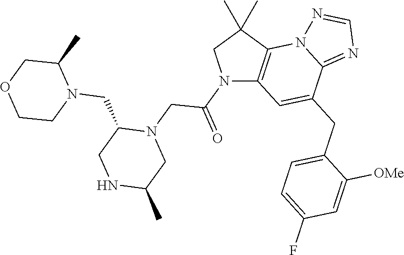 [SMILES: COc1cc(F)ccc1Cc1cc2c(n3ncnc13)C(C)(C)CN2C(=O)CN1C[C@@H](C)NC[C@@H]1CN1CCOC[C@H]1C]

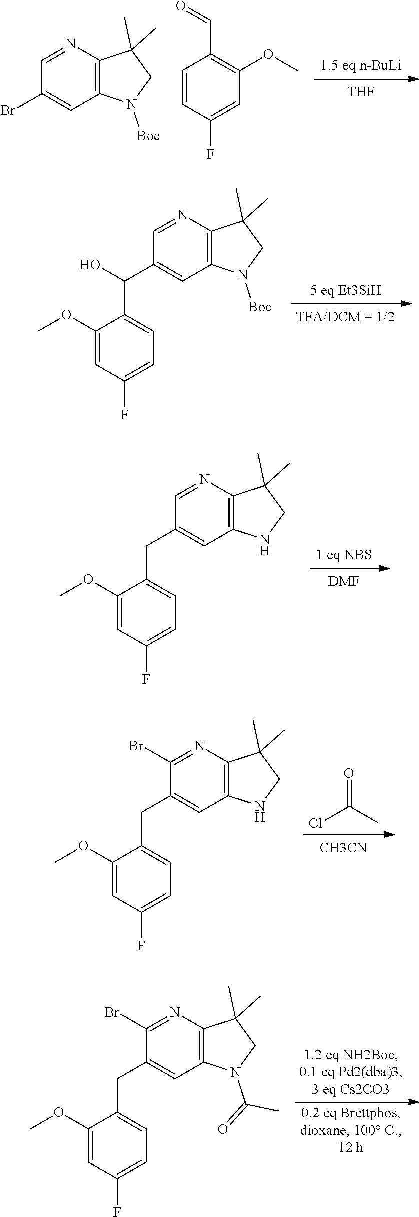

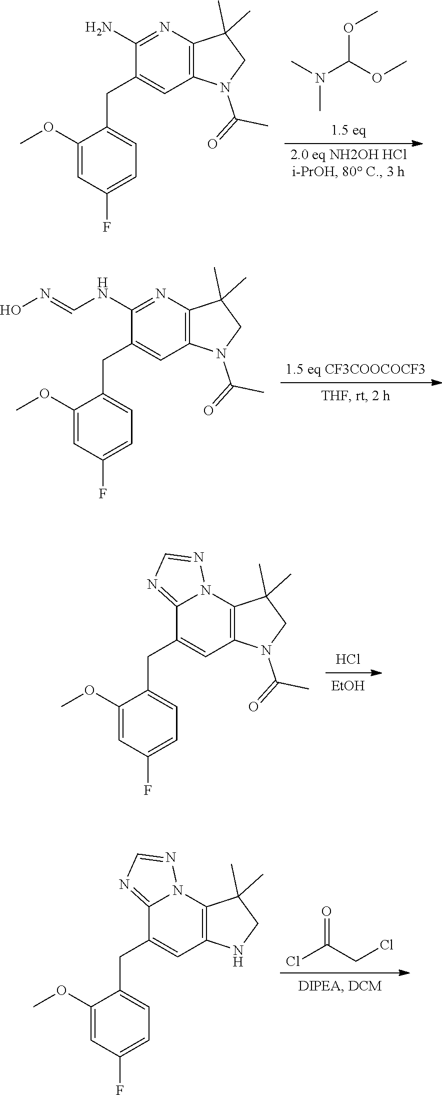

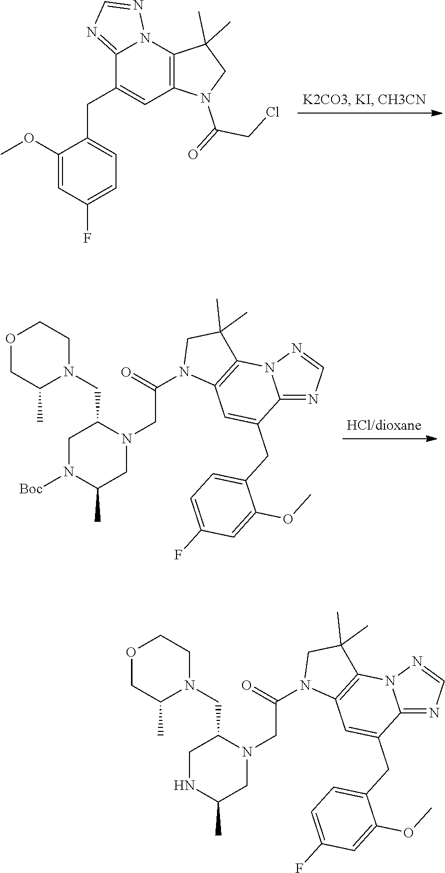

Step 1: tert-butyl 6-((4-fluoro-2-methoxyphenyl)(hydroxy)methyl)-3,3-dimethyl-2,3-dihydro-1H-pyrrolo[3,2-b]pyridine-1-carboxylate

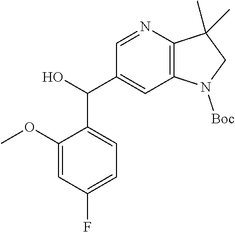

To a solution of tert-butyl 6-bromo-3,3-dimethyl-2,3-dihydro-1H-pyrrolo[3,2-b]pyridine-1-carboxylate (3 g, 9.17 mmol, 1.0 eq) in THF (15 mL) was added butyllithium (0.881 g, 13.75 mmol, 1.5 eq) at −78° C. under N₂ and stirred at −78° C. for 30 min, then 5-fluoro-2-methoxy benzaldehyde (2.120 g, 13.75 mmol, 1.5 eq) was added at −78° C. and stirred at −78° C. for 1 h. To the resultant were added saturated NH₄Cl (aq) and ethyl acetate. The organic layer was washed with brine (100 mL), dried over Na₂SO₄, filtered and concentrated. The crude product was added to a silica gel column and was eluted with PE/EA=2/1 to give the title compound (3.3 g, 89%) as a white solid.

Step 2: 6-(4-fluoro-2-methoxybenzyl)-3,3-dimethyl-2,3-dihydro-1H-pyrrolo[3,2-b]pyridine

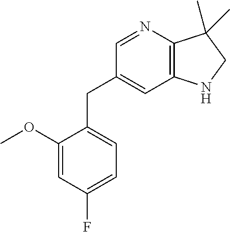

To a solution of tert-butyl 6-((4-fluoro-2-methoxyphenyl)(hydroxy)methyl)-3,3-dimethyl-2,3-dihydro-1H-pyrrolo[3,2-b]pyridine-1-carboxylate (2.4 g, 5.96 mmol, 1.0 eq) in DCM (16 mL) were added triethylsilane (3.47 g, 29.8 mmol, 5.0 eq) and TFA (8.00 mL) at 25° C. under N₂. The mixture was stirred at 25° C. for 6 h. After concentration, saturated NaHCO₃ (aq) and ethyl acetate were added. The organic layer was washed with brine (100 mL), dried over Na₂SO₄, filtered and concentrated. The crude product was added to a silica gel column and eluted with PE/EA=1/1 to give the title compound (1.6 g, 94%) as a white solid.

Step 3: 5-bromo-6-(4-fluoro-2-methoxybenzyl)-3,3-dimethyl-2,3-dihydro-1H-pyrrolo[3,2-b]pyridine

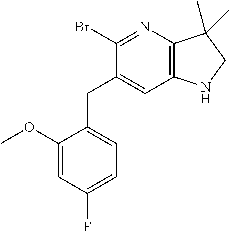

To a solution of 6-(4-fluoro-2-methoxybenzyl)-3,3-dimethyl-2,3-dihydro-1H-pyrrolo[3,2-b]pyridine (1.6 g, 5.59 mmol, 1.0 eq) in DMF (15 mL) was added 1-bromopyrrolidine-2,5-dione (0.995 g, 5.59 mmol, 1.0 eq) at 0° C. The mixture was stirred at 0° C. for 30 min and the sat NaHCO₃ aq and ethyl acetate were added. The organic layer was washed with brine (100 mL), dried over Na₂SO₄, filtered and concentrated, The crude product was added to a column and was eluted with PE/EA=1/1 to give the title compound (1.2 g, 58.8%) as a yellow solid. MS (ESI) m/z 366.5 [M+H]⁺

Step 4: 1-(5-bromo-6-(4-fluoro-2-methoxybenzyl)-3,3-dimethyl-2,3-dihydro-1H-pyrrolo[3,2-b]pyridin-1-yl)ethan-1-one

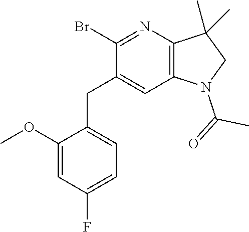 [SMILES: COc1cc(F)ccc1Cc1cc2c(nc1Br)C(C)(C)CN2C(C)=O]

To a solution of 5-bromo-6-(4-fluoro-2-methoxybenzyl)-3,3-dimethyl-2,3-dihydro-1H-pyrrolo[3,2-b]pyridine (500 mg, 1.369 mmol, 1.0 eq) in acetonitrile (15 mL) was added acetyl chloride (129 mg, 1.643 mmol, 1.2 eq) at 0° C. under N₂, then the mixture was stirred at 0° C. for 30 min. To the mixture was added saturated NaHCO₃(aq), the solution was extracted with ethyl acetate and the organic layer was washed with brine (100 mL), dried over Na₂SO₄, filtered and concentrated, The residue was purified by silica gel flash column chromatography eluting with PE/EA=1/1 to give the title compound (547 mg, 98%) as a white solid.

Step 5: 1-(5-amino-6-(4-fluoro-2-methoxybenzyl)-3,3-dimethyl-2,3-dihydro-1H-pyrrolo[3,2-b]pyridin-1-yl)ethan-1-one

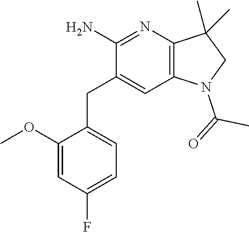 [SMILES: COc1cc(F)ccc1Cc1cc2c(nc1N)C(C)(C)CN2C(C)=O]

To a solution of 1-(5-bromo-6-(4-fluoro-2-methoxybenzyl)-3,3-dimethyl-2,3-dihydro-1H-pyrrolo[3,2-b]pyridin-1-yl)ethan-1-one (547 mg, 1.343 mmol, 1.0 eq) in dioxane (15 mL) were added tert-butyl carbamate (189 mg, 1.612 mmol, 1.2 eq), dicyclohexyl(2′,4′,6′-triisopropyl-3,6-dimethoxy-[1,1′-biphenyl]-2-yl)phosphane (144 mg, 0.269 mmol, 0.2 eq), Cs₂CO₃ (1313 mg, 4.03 mmol, 3.0 eq), and Pd₂(dba)₃ (123 mg, 0.134 mmol, 0.1 eq) at 25° C. The mixture was heated to 100° C. overnight under N₂. After concentration, the residue was purified by silica gel flash column chromatography eluted with PE/EA=1/2 to give the title compound (175 mg, 37.9%) as a yellow solid. MS (ESI) m/z 344.8 [M+H]⁺.

Step 6: (E)-N-(1-acetyl-6-(4-fluoro-2-methoxybenzyl)-3,3-dimethyl-2,3-dihydro-1H-pyrrolo[3,2-b]pyridin-5-yl)-N′-hydroxyformimidamide)

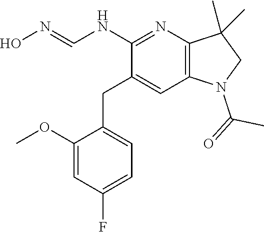 [SMILES: COc1cc(F)ccc1Cc1cc2c(nc1N/C=N/O)C(C)(C)CN2C(C)=O]

To a solution of 1-(5-amino-6-(4-fluoro-2-methoxybenzyl)-3,3-dimethyl-2,3-dihydro-1H-pyrrolo[3,2-b]pyridin-1-yl)ethan-1-one (175 mg, 0.510 mmol, 1.0 eq) in 2-propanol (15 mL) was added 1,1-dimethoxy-N,N-dimethylmethanamine (91 mg, 0.764 mmol, 1.5 eq) at 25° C., The mixture was heated to 80° C. for 2 h under N₂, then hydroxylamine hydrochloride (70.8 mg, 1.019 mmol, 2.0 eq) was added at room temperature. The mixture was heated to 50° C. for 1 h under N₂. After concentration, the residue was used in next step directly as a yellow solid. MS (ESI) m/z 388.0 [M+H]⁺.

Step 7: 1-(4-(4-fluoro-2-methoxybenzyl)-8,8-dimethyl-7,8-dihydro-6H-pyrrolo[2,3-e][1,2,4]triazolo[1,5-a]pyridin-6-yl)ethan-1-one

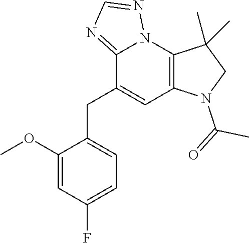 [SMILES: COc1cc(F)ccc1Cc1cc2c(n3ncnc13)C(C)(C)CN2C(C)=O]

To a solution of (E)-N-(1-acetyl-6-(4-fluoro-2-methoxybenzyl)-3,3-dimethyl-2,3-dihydro-1H-pyrrolo[3,2-b]pyridin-5-yl)-N′-hydroxyformimidamide (197 mg, 0.510 mmol, 1.0 eq) in THF (15 mL) was added 2,2,2-trifluoroacetic anhydride (161 mg, 0.765 mmol, 1.5 eq) at 25° C. The mixture was stirred at 25° C. for 2 h under N₂. The solution was concentrated and the residue was purified by silica gel flash column chromatography eluting with DCM/CH3OH=20/1 to give the title compound (119 mg, 63.4%) as a white solid. MS (ESI) m/z 369.8 [M+H]⁺.

Step 8: 4-(4-fluoro-2-methoxybenzyl)-8,8-dimethyl-7,8-dihydro-6H-pyrrolo[2,3-e][1,2,4]triazolo[1,5-a]pyridine

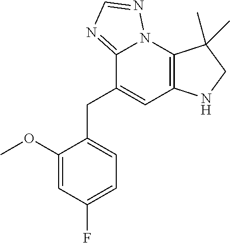

To a solution of 1-(4-(4-fluoro-2-methoxybenzyl)-8,8-dimethyl-7,8-dihydro-6H-pyrrolo[2,3-e][1,2,4]triazolo[1,5-a]pyridin-6-yl)ethan-1-one (119 mg, 0.323 mmol, 1.0 eq) in EtOH (10 mL) was added 5 N HCl (2 mL) at 25° C. The mixture was stirred at 80° C. for 2 h. After concentration, saturated NaHCO₃(aq) and ethyl acetate were added. The organic layer was washed with brine (100 mL), dried over Na₂SO₄, filtered and concentrated. The crude product was purified by silica gel flash column chromatography eluted with PE/EA=1/1 to give the title compound (100 mg, 95%) as a yellow solid. MS (ESI) m/z 327.8 [M+H]⁺.

Step 9: 2-chloro-1-(4-(4-fluoro-2-methoxybenzyl)-8,8-dimethyl-7,8-dihydro-6H-pyrrolo[2,3-e][1,2,4]triazolo[1,5-a]pyridin-6-yl)ethan-1-one

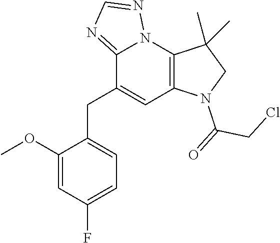 [SMILES: COc1cc(F)ccc1Cc1cc2c(n3ncnc13)C(C)(C)CN2C(=O)CCl]

To a solution of 4-(4-fluoro-2-methoxybenzyl)-8,8-dimethyl-7,8-dihydro-6H-pyrrolo[2,3-e][1,2,4]triazolo[1,5-a]pyridine (100 mg, 0.306 mmol, 1.0 eq) in CH₂Cl₂ (15 mL) were added N-ethyl-N-isopropylpropan-2-amine (119 mg, 0.919 mmol, 3.0 eq) and 2-chloroacetyl chloride (41.5 mg, 0.368 mmol, 1.2 eq) sequentially at 0° C. and the mixture was stirred at rt for 3 h under N₂. To the reaction mixture were added water and DCM. The combined organic layer was washed with brine (100 mL), dried over Na₂SO₄, filtered and concentrated. The residue was purified by silica gel flash column chromatography eluting with PE/EA=1/1 to give the title compound (120 mg, 97%) as a white solid. MS (ESI) m/z 404.5 [M+H]⁺.

Step 10: tert-butyl (2R,5S)-4-(2-(4-(4-fluoro-2-methoxybenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

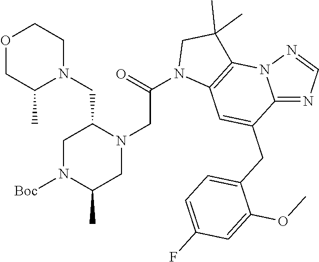 [SMILES: COc1cc(F)ccc1Cc1cc2c(n3ncnc13)C(C)(C)CN2C(=O)CN1C[C@@H](C)N(C(=O)OC(C)(C)C)C[C@@H]1CN1CCOC[C@H]1C]

To a solution of 2-chloro-1-(4-(4-fluoro-2-methoxybenzyl)-8,8-dimethyl-7,8-dihydro-6H-pyrrolo[2,3-e][1,2,4]triazolo[1,5-a]pyridin-6-yl)ethan-1-one (120 mg, 0.298 mmol, 1.0 eq) in acetonitrile (15 mL) were added tert-butyl-(2R,5S)-2-methyl-5-(((R)-3-methylmorpholino) methyl) piperazine-1-carboxylate II (93 mg, 0.298 mmol, 1.0 eq), potassium iodide (74.2 mg, 0.447 mmol, 1.5 eq) and K₂CO₃ (124 mg, 0.894 mmol, 3.0 eq) at 25° C. and the mixture was stirred at rt for 3 h under N₂. Water and ethyl acetate were added. The combined organic layer was washed with brine (100 mL), dried over Na₂SO₄, filtered and concentrated. The residue was purified by silica gel flash column chromatography eluting with DCM/CH₃OH=50/1 to give the title compound (195 mg, 96%) as a yellow solid. MS (ESI) m/z 681.1 [M+H]⁺.

Step 11: 1-(4-(4-fluoro-2-methoxybenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl) piperazin-1-yl)ethan-1-one (Compound 41)

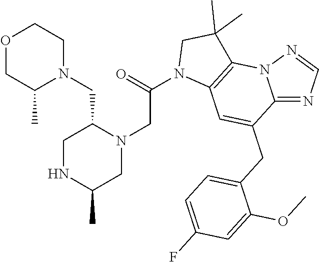 [SMILES: COc1cc(F)ccc1Cc1cc2c(n3ncnc13)C(C)(C)CN2C(=O)CN1C[C@@H](C)NC[C@@H]1CN1CCOC[C@H]1C]

A solution of tert-butyl (2R,5S)-4-(2-(4-(4-fluoro-2-methoxybenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (195 mg, 0.287 mmol, 1.0 eq) in HCl/dioxane (3 mL) was stirred at 25 CC for 30 min. The solvent was evaporated and 7.0 N NH₃ in CH₃OH (5 mL) was added. After concentration, the residue was purified by silica gel flash column chromatography eluted with DCM/CH₃OH=5/1 to give the title compound (144 mg, 87%) as a white solid. MS (ESI) m/z 580.6[M+H]. ¹H NMR (400 MHz, DMSO-d6) δ (ppm) 8.48 (s, 1H), 8.22 (s, 1H), 7.29-7.17 (m, 1H), 6.99-6.84 (m, 1H), 6.78-6.60 (m, 1H), 4.23-4.11 (m, 3H), 3.92 (d, J=10.6 Hz, 1H), 3.82-3.74 (m, 4H), 3.60 (d, J=16.5 Hz, 1H), 3.49-3.41 (m, 2H), 3.18-3.07 (m, 1H), 2.96-2.75 (m, 4H), 2.72-2.65 (m, 1H), 2.61-2.52 (m, 2H), 2.42-2.33 (m, 1H), 2.31-2.21 (m, 1H), 2.13 (m, 1H), 1.97-1.84 (m, 1H), 1.79-1.68 (m, 1H), 1.58 (d, J=17.1 Hz, 6H), 0.87 (m, 6H).

Example 39: 1-(4-(4-fluoro-2-hydroxybenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 42)

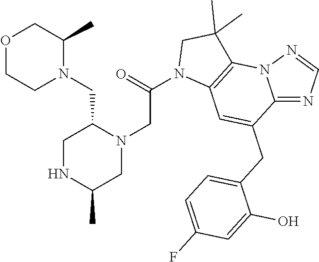 [SMILES: C[C@@H]1CN(CC(=O)N2CC(C)(C)c3c2cc(Cc2ccc(F)cc2O)c2ncnn32)[C@@H](CN2CCOC[C@H]2C)CN1]

To a solution of 1-(4-(4-fluoro-2-methoxybenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino) methyl) piperazin-1-yl) ethan-1-one (30 mg, 0.052 mmol, 1.0 eq) in CH₂Cl₂ (15 mL) was added 1 N tribromoborane (0.5 mL) at 0° C. The mixture was stirred at 25° C. for 5 h under N₂. Saturated NaHCO₃(aq) was added. The solution was extracted with ethyl acetate and the organic layer was washed with brine (100 mL), dried over Na₂SO₄, filtered and concentrated, The residue was purified by pre-HPLC to give the title compound (5.1 mg, 17.42%) as a yellow solid. MS (ESI) m/z 566.3[M+H]^{+ 1}H NMR (400 MHz, DMSO-d⁶) δ (ppm) 8.49 (s, 1H), 8.29 (s, 1H), 7.27-7.13 (m, 1H), 6.62-6.48 (m, 2H), 4.23-4.06 (m, 3H), 3.91 (d, J=10.6 Hz, 1H), 3.78 (d, J=16.6 Hz, 1H), 3.63 (d, J=16.6 Hz, 1H), 3.47-3.41 (m, 2H), 3.15-3.04 (m, 1H), 3.01-2.91 (m, 1H), 2.91-2.73 (m, 3H), 2.73-2.62 (m, 1H), 2.63-2.54 (m, 2H), 2.46-2.38 (m, 1H), 2.33-2.18 (m, 1H), 2.17-2.03 (m, 1H), 1.97-1.83 (m, 1H), 1.79-1.67 (m, 1H), 1.57 (d, J=19.7 Hz, 6H), 0.92-0.81 (m, 6H).

Example 40: 1-(4-(4-chlorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 44)

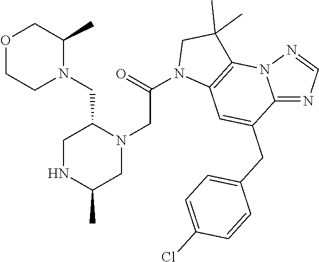 [SMILES: C[C@@H]1CN(CC(=O)N2CC(C)(C)c3c2cc(Cc2ccc(Cl)cc2)c2ncnn32)[C@@H](CN2CCOC[C@H]2C)CN1]

Compound 44 was synthesized in a manner similar to that described in compound 14. ¹H NMR (400 MHz, DMSO-d⁶) δ (ppm) 8.50 (s, 1H), 8.40 (s, 1H), 7.45-7.28 (m, 4H), 4.34-4.22 (m, 2H), 4.15 (d, J=10.6 Hz, 1H), 3.97-3.89 (m, 1H), 3.85-3.76 (m, 1H), 3.68-3.58 (m, 1H), 3.49-3.44 (m, 2H), 3.22-3.07 (m, 1H), 2.99-2.79 (m, 4H), 2.74-2.66 (m, 1H), 2.65-2.54 (m, 2H), 2.46-2.38 (m, 1H), 2.34-2.23 (m, 1H), 2.20-2.08 (m, 1H), 1.99-1.90 (m, 1H), 1.79-1.70 (m, 1H), 1.58 (d, J=16.6 Hz, 6H), 0.91-0.85 (m, 6H).

Example 41: 1-(4-(1-(4-fluorophenyl)ethyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-m ethylmorpholino) methyl)piperazin-1-yl)ethan-1-one (Compound 45)

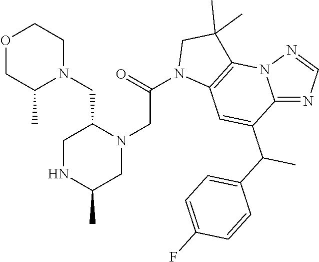 [SMILES: CC(c1ccc(F)cc1)c1cc2c(n3ncnc13)C(C)(C)CN2C(=O)CN1C[C@@H](C)NC[C@@H]1CN1CCOC[C@H]1C]

Compound 45 was synthesized in a manner similar to that described in compound 41. MS (ESI) m/z 564.7[M+H]⁺. ¹H NMR (400 MHz, DMSO-d⁶) δ (ppm) 8.57-8.37 (m, 2H), 7.47-7.26 (m, 2H), 7.16-7.01 (m, 2H), 4.90-4.70 (m, 1H), 4.23-4.09 (m, 1H), 4.00-3.89 (m, 1H), 3.86-3.75 (m, 1H), 3.75-3.62 (m, 1H), 3.58-3.38 (m, 3H), 3.23-3.15 (m, 1H), 3.13-2.97 (m, 2H), 2.96-2.77 (m, 3H), 2.75-2.67 (m, 1H), 2.66-2.56 (m, 2H), 2.35-2.22 (m, 1H), 2.22-2.06 (m, 1H), 2.01-1.80 (m, 1H), 1.78-1.48 (m, 9H), 0.96-0.78 (m, 6H).

Example 42: 1-(4-((4-fluorophenyl)thio)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 49)

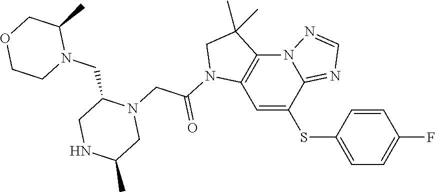 [SMILES: C[C@@H]1CN(CC(=O)N2CC(C)(C)c3c2cc(Sc2ccc(F)cc2)c2ncnn32)[C@@H](CN2CCOC[C@H]2C)CN1]

Compound 49 was synthesized in a manner similar to that described in compound 168. ¹H NMR (400 MHz, DMSO-d⁶) δ (ppm) 8.57 (s, 1H), 8.14 (s, 1H), 7.72-7.60 (m, 2H), 7.45-7.33 (m, 2H), 4.21 (d, J=10.4 Hz, 1H), 3.97 (d, J=10.4 Hz, 1H), 3.85 (d, J=16.4 Hz, 1H), 3.62-3.47 (m, 3H), 3.27-3.13 (m, 2H), 3.00-2.60 (m, 7H), 2.41-2.26 (m, 2H), 2.23-2.13 (m, 1H), 2.00-1.91 (m, 1H), 1.83-1.73 (m, 1H), 1.62 (d, J=16.4 Hz, 6H), 0.98-0.83 (m, 6H). MS (ESI) m/z 568.50 [M+H]⁺.

Example 43: 1-(4-((2,4-difluorophenyl)(hydroxy)methyl)-8,8-dimethyl-7,8-dihydro-6H-pyrrolo[2,3-e][1,2,4]triazolo[1,5-a]pyridin-6-yl)-2-((2R,5R)-5-meth yl-2-(((R)-3-methylmorpholino)methyl)piperazin-1-yl)ethan-1-one (Compound 50)

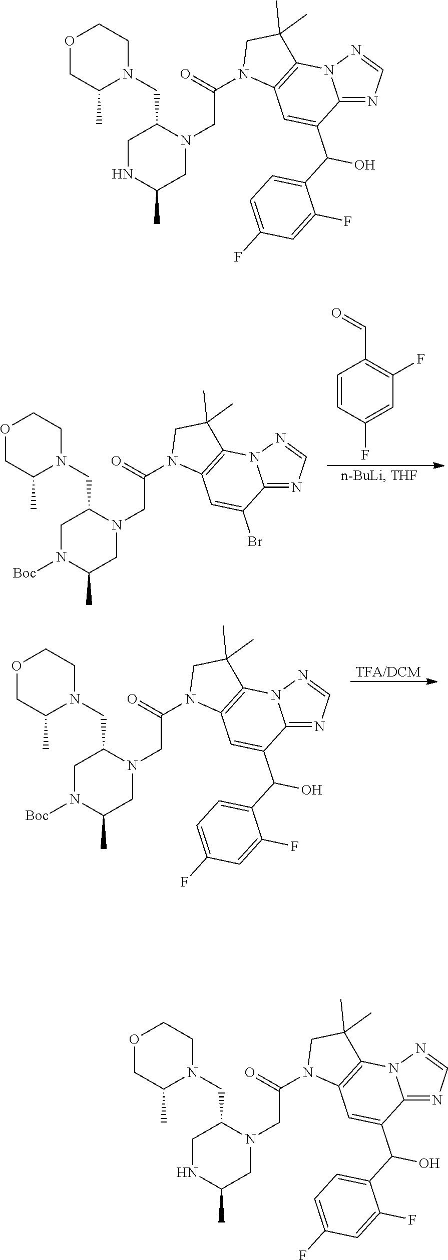

Step 1: tert-butyl (2R,5S)-4-(2-(4-((2,4-difluorophenyl)(hydroxy)methyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

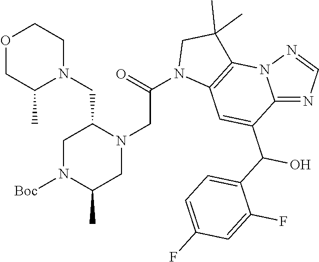 [SMILES: C[C@@H]1COCCN1C[C@H]1CN(C(=O)OC(C)(C)C)[C@H](C)CN1CC(=O)N1CC(C)(C)c2c1cc(C(O)c1ccc(F)cc1F)c1ncnn21]

To a solution of tert-butyl (2R,5S)-4-(2-(4-bromo-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl) piperazine-1-carboxylate (50 mg, 0.081 mmol, 1.0 eq) in THF (15 mL) was added 2.5 N butyllithium (4.8 mL, 0.121 mmol, 1.5 eq) at −78° C. under N₂. The mixture was stirred at −78° C. for 1 h. 2,4-difluorobenzaldehyde (17.17 mg, 0.121 mmol, 1.5 eq) was added at −78° C. and stirred at −78° C. for 2 h. Saturated NH₄Cl (aq) and ethyl acetate were added. The combined organic layer was washed with brine (100 mL), dried over Na₂SO₄, filtered and concentrated. The residue was purified by silica gel flash column chromatography eluting with DCM/CH₃OH=50/1 to give the title compound (17 mg, 30.9%) as a yellow solid. MS (ESI) m/z 684.6 [M+H]⁺.

Step 2: Compound 50 was obtained as a white solid after normal acidic deprotection conditions. MS (ESI) m/z 584.6 [M+H]⁺.

Example 44: 1-(4-(2-cyclopropyl-1,1-difluoroethyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 53)

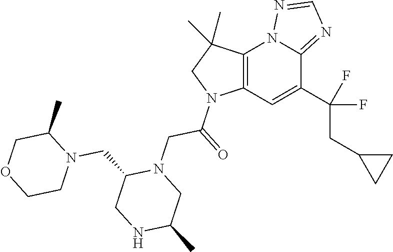 [SMILES: C[C@@H]1CN(CC(=O)N2CC(C)(C)c3c2cc(C(F)(F)CC2CC2)c2ncnn32)[C@@H](CN2CCOC[C@H]2C)CN1]

Compound 53 was synthesized as a white solid (11.6 mg) in a manner similar to that described in compound 166. ¹H NMR (400 MHz, DMSO-d⁶) δ (ppm) 8.79 (s, 1H), 8.62 (s, 1H), 4.22 (d, J=10.6 Hz, 1H), 4.03 (d, J=10.6 Hz, 1H), 3.86 (d, J=16.7 Hz, 1H), 3.72 (d, J=16.7 Hz, 1H), 3.60 (t, J=6.5 Hz, 1H), 3.53-3.47 (m, 2H), 3.22-3.18 (m, 1H), 3.07-3.01 (m, 1H), 2.95-2.82 (m, 3H), 2.76 (d, J=9.6 Hz, 1H), 2.69-2.63 (m, 2H), 2.61-2.54 (m, 2H), 2.35-2.29 (m, 1H), 2.20-2.12 (m, 1H), 1.99-1.93 (m, 1H), 1.83-1.77 (m, 1H), 1.64 (d, J=12.9 Hz, 6H), 0.92 (t, J=6.0 Hz, 6H), 0.68-0.55 (m, 1H), 0.38-0.27 (m, 2H), 0.11-−0.09 (m, 2H). MS (ESI) m/z 546.5 [M+H]⁺.

Example 45: 1-(4′-(4-fluorobenzyl)spiro[cyclopropane-1,8′-pyrrolo[2,3-e][1,2,4]triazolo [1,5-a]pyridin]-6′(7′H)-yl)-2-((2R,5R)-5-methyl-2-(((R)-3-methylmorpholino)methyl) piperazin-1-yl)ethan-1-one (Compound 56)

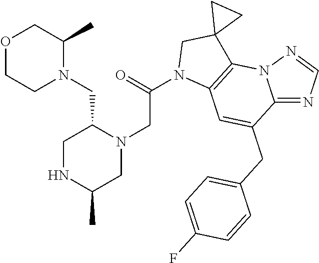 [SMILES: C[C@@H]1CN(CC(=O)N2CC3(CC3)c3c2cc(Cc2ccc(F)cc2)c2ncnn32)[C@@H](CN2CCOC[C@H]2C)CN1]

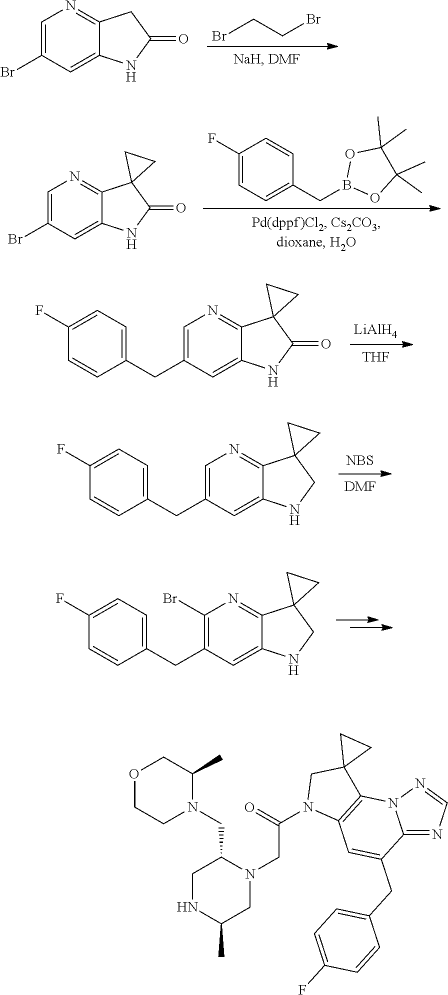

Step 1: 6′-bromospiro[cyclopropane--1,3′-pyrrolo[3,2-b]pyridin]-2′(1′H)-one

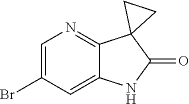 [SMILES: O=C1Nc2cc(Br)cnc2C12CC2]

6-Bromo-1,3-dihydro-2H-pyrrolo[3,2-b]pyridin-2-one (19.0 g, 1.0 eq) was dissolved in 200 mL DMF. NaH (9.0 g, 2.5 eq) was added dropwise at 0-5° C. The reaction mixture was stirred at 0-5° C. for 1-1.5 h. 1,2-Dibromoethane (25.0 g, 1.5 eq) was added dropwise at 0-5° C. The reaction was warmed to room temperature and stirred at room temperature 16 h. The reaction was poured into 1000 mL ice water and extracted with ethyl acetate four times. The combined organic phase was washed with water, brine, dried over Na₂SO₄, filtrated and concentrated. The residue was purified by chromatography to afford the title compound (8.5 g, 40% yield). LC-MS: [M+H]⁺ 239.2.

Step 2: 6′-(4-fluorobenzyl)spiro cyclopropane-1,3′-pyrrolo[3,2-b]pyridin]-2′(1′H)-one

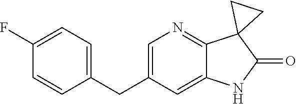 [SMILES: O=C1Nc2cc(Cc3ccc(F)cc3)cnc2C12CC2]

Above intermediate (8.5 g, 1.0 eq) 2-(4-fluorophenyl)-4,4,5,5-tetramethyl-1,3,2-dioxaborolane (31.5 g, 4.0 eq), Cs₂CO₃ (57.9 g, 6.0 eq) was added in 80 mL of dioxane and 15 mL of H₂O. Pd(dppf)Cl₂ (5.2 g, 0.2 eq) was added. The reaction was heated to 90° C. and reacted for 4 h under N₂ atmosphere. The reaction was cooled to room temperature and concentrated. The residue was purified by chromatography to afford the title compound (7.5 g, 78% yield).

Step 3: 6′-(4-fluorobenzyl)-1′,2′-dihydrospiro[cyclopropane-1,3′-pyrrolo[3,2-b]pyridine]

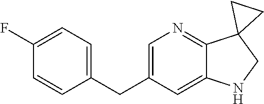 [SMILES: Fc1ccc(Cc2cnc3c(c2)NCC32CC2)cc1]

Above intermediate (7.5 g, 1.0 eq) was dissolved in 80 mL THF. LiAlH₄ (4.24 g, 4.0 eq) was added dropwise at −10˜−5° C. The reaction mixture was stirred for 30 min and then heated to 50° C. for 1 h. Then reaction was cooled to 0-5° C. 4 mL of H₂O, 4 mL of 15% NaOH aqueous solution, 12 mL of H₂O was added sequentially. The solution was filtrated and filter cake was washed by ethyl acetate three times. The combined filtrate was concentrated and purified by silica gel chromatography to afford the title compound (5.2 g, 73% yield). LC-MS: [M+H]⁺ 255.2.

Step 4: 5′-bromo-6′-(4-fluorobenzyl)-1′,2′-dihydrospiro[cyclopropane-1,3′-pyrrolo[3,2-b]pyridine]

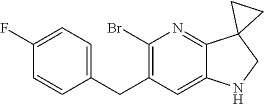 [SMILES: Fc1ccc(Cc2cc3c(nc2Br)C2(CC2)CN3)cc1]

Above intermediate (800 mg, 1.0 eq) was dissolved in 10 mL of DMF. NBS (589 mg, 1.05 eq) in 5 mL of DMF was added dropwise at −20° C. The reaction was stirred for 2 h and quenched with 50 mL of H₂O. The mixture was extracted with ethyl acetate for four times. The combined organic phase was washed with water, brine, dried over Na₂SO₄. The solution was filtrated and concentrated. The residue was purified by silica gel chromatography to afford the title compound (460 mg, 44% yield). ¹H NMR (400 MHz, Chloroform-d) δ (ppm) 7.23-7.10 (m, 2H), 7.07-6.91 (m, 2H), 6.51 (s, 1H), 3.95 (s, 2H), 3.81 (brs, 1H), 3.70 (s, 2H), 1.36 (q, J=4.3 Hz, 2H), 0.96 (q, J=4.3 Hz, 2H). HPLC-MS: [M+H]⁺=333.20/335.20 found.

Following steps: compound 56 was synthesized as a white solid (30.6 mg) in a manner similar to that described in compound 14. MS (ESI) m/z 548.5[M+H]⁺. ¹H NMR (400 MHz, DMSO-d⁶) δ (ppm) 8.42 (s, 1H), 8.38 (s, 1H), 7.41-7.26 (m, 2H), 7.13-7.00 (m, 2H), 4.35-4.23 (m, 3H), 4.22-4.11 (m, 1H), 3.74-3.56 (m, 2H), 3.49-3.40 (m, 2H), 3.14-3.05 (m, 1H), 3.00-2.92 (m, 1H), 2.91-2.73 (m, 4H), 2.69-2.64 (m, 1H), 2.60-2.55 (m, 2H), 2.34-2.20 (m, 1H), 2.18-2.09 (m, 1H), 2.05-1.98 (m, 2H), 1.96-1.82 (m, 1H), 1.80-1.66 (m, 1H), 1.17-1.07 (m, 2H), 0.93-0.80 (m, 7H).

Example 46: 1-(4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-2-methyl-4-(2,2,2-trifluoroethyl)piperazin-1-yl)methyl)piperazin-1-yl)ethan-1-one (Compound 63)

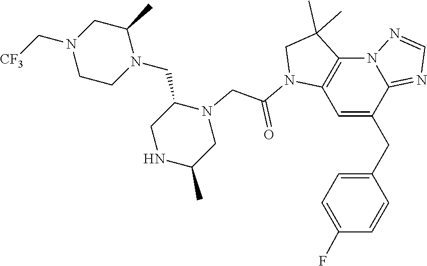 [SMILES: C[C@@H]1CN(CC(=O)N2CC(C)(C)c3c2cc(Cc2ccc(F)cc2)c2ncnn32)[C@@H](CN2CCN(CC(F)(F)F)C[C@H]2C)CN1]

Compound 63 was synthesized as a white solid (57 mg) in a manner similar to that described in compound 174. ¹H NMR (400 MHz, DMSO-d⁶) δ (ppm) 8.50 (d, J=0.9 Hz, 1H), 8.38 (s, 1H), 7.39 (dd, J=8.4, 5.7 Hz, 2H), 7.10 (t, J=8.4 Hz, 2H), 4.35-4.23 (m, 2H), 4.14 (d, J=10.5 Hz, 1H), 3.93 (d, J=10.6 Hz, 1H), 3.83 (d, J=16.6 Hz, 1H), 3.70-3.58 (m, 1H), 3.04-2.90 (m, 2H), 2.89-2.70 (m, 5H), 2.67-2.57 (m, 3H), 2.47-2.42 (m, 1H), 2.35-2.27 (m, 1H), 2.25-2.14 (m, 2H), 2.03 (t, J=9.5 Hz, 1H), 1.93 (t, J=10.6 Hz, 1H), 1.75 (d, J=13.0 Hz, 1H), 1.59 (d, J=19.3 Hz, 6H), 0.96 (d, J=6.0 Hz, 3H), 0.93-0.87 (m, 3H). MS (ESI) m/z 631.6 [M+H]⁺.

Example 47: 2-((2R,5R)-2-(((R)-4,4-difluoro-2-methylpiperidin-1-yl)methyl)-5-methylpiperazin-1-yl)-1-(4-(4-fluorobenzyl)-8,8-dimethyl-7,8-dihydro-6H-pyrrolo[2,3-e][1,2,4]triazolo[1,5-a]pyridin-6-yl)ethan-1-one (compound 75)

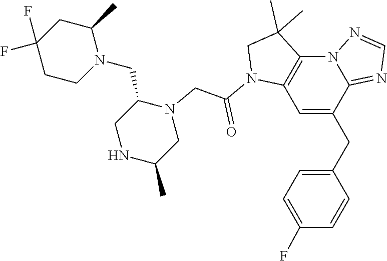 [SMILES: C[C@@H]1CN(CC(=O)N2CC(C)(C)c3c2cc(Cc2ccc(F)cc2)c2ncnn32)[C@@H](CN2CCC(F)(F)C[C@H]2C)CN1]

Compound 75 was synthesized as a white solid (27 mg) in a manner similar to that described in compound 174. MS (ESI) m/z 584.5 [M+H]⁺.

Example 48: 1-(4-(4-fluorobenzyl)-8,8-dimethyl-7,8-dihydro-6H-pyrrolo[2,3-e][1,2,4]triazolo[1,5-a]pyridin-6-yl)-2-((2R,5R)-2-(((2R)-4-hydroxy-2,4-dimethylpiperidin-1-yl)methyl)-5-methylpiperazin-1-yl)ethan-1-one (compound 77)

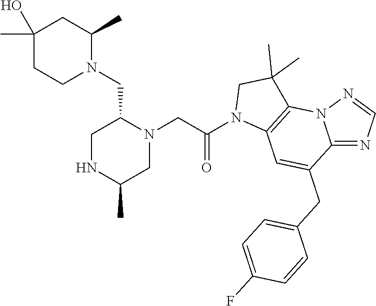 [SMILES: C[C@@H]1CN(CC(=O)N2CC(C)(C)c3c2cc(Cc2ccc(F)cc2)c2ncnn32)[C@@H](CN2CCC(C)(O)C[C@H]2C)CN1]

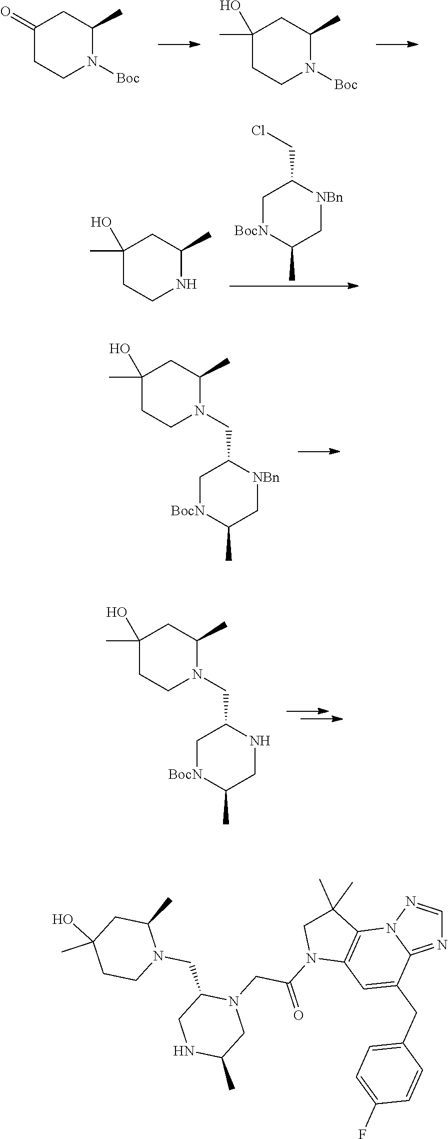

Step 1: tert-butyl (2R)-4-hydroxy-2,4-dimethylpiperidine-1-carboxylate

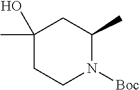 [SMILES: C[C@@H]1CC(C)(O)CCN1C(=O)OC(C)(C)C]

To a solution of tert-butyl (R)-2-methyl-4-oxopiperidine-1-carboxylate (0.5 g, 2.34 mmol, 1.0 eq) in dry THF (10 mL) was added MeMgBr (4.8 mL, 23.4 mmol, 10.0 eq) slowly at −78° C. Then mixture was stirred 30 min. The mixture was warmed to rt and stirred for 18 h. The mixture was quenched by NH₄Cl solution, diluted by ethyl acetate (200 mL). the organic phase was collected, dried by anhydrous Na₂SO₄. After concentration, the residue was directly used in the next step. (0.50 g, crude).

Step 2: (2R)-2,4-dimethylpiperidin-4-ol

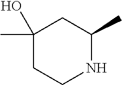

Above intermediate (0.5 g, 2.18 mmol) in conc. hydrogen chloride (1 mL) and MeOH (5 mL) was stirred for 2 h at rt. The solvent was removed to afford the title compound (0.25 g, crude) which was directly used in the next step.

Step 3: tert-butyl (2R,5S)-4-benzyl-5-(((2R)-4-hydroxy-2,4-dimethylpiperidin-1-yl)methyl)-2-methylpiperazine-1-carboxylate

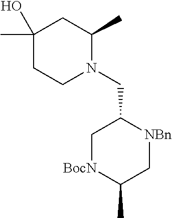 [SMILES: C[C@@H]1CC(C)(O)CCN1C[C@H]1CN(C(=O)OC(C)(C)C)[C@H](C)CN1Cc1ccccc1]

A vial was charged with above intermediate (0.25 g, 1.0 eq), tert-butyl (2R,5R)-4-benzyl-5-(chloromethyl)-2-methylpiperazine-1-carboxylate (0.25 g, 0.4 eq), Cs₂CO₃ (0.68 g, 1.0 eq), KI (0.04 g, 0.1 eq) and CH₃CN (10 mL). The mixture was purged with nitrogen and heated to 80° C. for 15 h. LCMS showed the reaction was complete. The mixture was diluted with 50 mL H₂O and extracted with ethyl acetate and dried over sodium sulfate, filtered and concentrated in vacuum. The residue was purified by silica gel chromatography to give the title compound (0.28 g, 55%). LC-MS (m/z): 432.1 [M+H]⁺.

Step 4: tert-butyl (2R,5S)-5-(((2R)-4-hydroxy-2,4-dimethylpiperidin-1-yl)methyl)-2-methylpiperazine-1-carboxylate

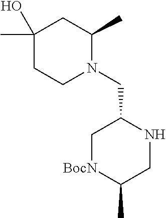 [SMILES: C[C@@H]1CC(C)(O)CCN1C[C@H]1CN(C(=O)OC(C)(C)C)[C@H](C)CN1]

To a solution of above intermediate (0.28 g, 1 eq.) in CF₃CH₂OH (15 mL) was added Pd/C (0.2 g, 10% on Carbon, wetted with ca. 55% water) and the mixture was stirred at rt for 5 hours under H₂ (balloon). TLC were done to detect the process of the reaction. Once the reaction was completed, the catalyst was removed by filtration and the filtrate was concentrated to give the title compound (0.19 g, 86%). LC-MS: 342.42 [M+H]⁺. ¹H NMR (600 MHz, Chloroform-d) δ (ppm) 4.16 (brs, 1H), 3.63 (d, J=13.7 Hz, 1H), 3.30 (dd, J=13.7, 3.8 Hz, 1H), 3.12-3.00 (m, 3H), 2.93 (d, J=12.2 Hz, 1H), 2.50 (dd, J=12.4, 2.7 Hz, 1H), 2.10 (m, 2H), 1.91 (dd, J=11.8, 3.5 Hz, 1H), 1.71-1.57 (m, 3H), 1.45 (s, 9H), 1.26 (d, J=6.8 Hz, 3H), 1.24 (s, 3H), 1.07 (d, J=6.1 Hz, 3H).

Following steps: compound 77 was synthesized as a white solid (43.5 mg) in a manner similar to that described in compound 174. MS (ESI) m/z 578.6 [M+H]⁺.

Example 49: 2-((2R,5R)-2-((2-oxa-5-azabicyclo[2.2.1]heptan-5-yl)methyl)-5-methylpiperazin-1-yl)-1-(4-(4-fluorobenzyl)-8,8-dimethyl-7,8-dihydro-6H-pyrrolo[2,3-e][1,2,4]triazolo[1,5-a]pyridin-6-yl)ethan-1-one (Compound 78 and 79)

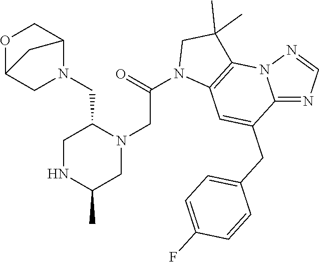 [SMILES: C[C@@H]1CN(CC(=O)N2CC(C)(C)c3c2cc(Cc2ccc(F)cc2)c2ncnn32)[C@@H](CN2CC3CC2CO3)CN1]

Compound 78 and 79 was synthesized as a mixture of diastereoisomers as a white solid (45 mg) in a manner similar to that described in compound 174. ¹H NMR (400 MHz, DMSO-d⁶) δ (ppm) 8.50 (s, 1H), 8.40 (s, 1H), 7.39 (dd, J=8.3, 5.7 Hz, 2H), 7.11 (t, J=8.8 Hz, 2H), 4.30 (s, 2H), 4.24-4.17 (m, 2H), 4.00 (d, J=10.6 Hz, 1H), 3.83 (d, J=16.0 Hz, 1H), 3.73 (d, J=7.3 Hz, 1H), 3.41 (s, 2H), 3.38 (s, 1H), 2.84 (d, J=9.5 Hz, 1H), 2.78-2.68 (m, 2H), 2.68-2.53 (m, 3H), 2.36-2.22 (m, 4H), 1.57 (d, J=10.2 Hz, 6H), 1.50 (d, J=9.2 Hz, 1H), 1.39 (d, J=9.1 Hz, 1H), 0.87 (d, J=6.1 Hz, 3H). MS (ESI) m/z 548.4 [M+H]⁺.

Example 50: 2-((2S,5R)-2-((8-oxa-3-azabicyclo[3.2.1]octan-3-yl)methyl)-5-methylpiperazin-1-yl)-1-(4-(4-fluorobenzyl)-8,8-dimethyl-7,8-dihydro-6H-pyrrolo[2,3-e][1,2,4]triazolo[1,5-a]pyridin-6-yl)ethan-1-one (Compound 80)

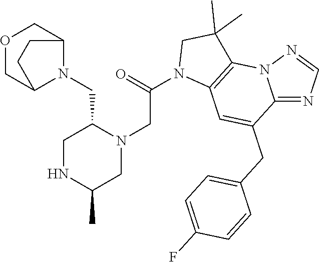 [SMILES: C[C@@H]1CN(CC(=O)N2CC(C)(C)c3c2cc(Cc2ccc(F)cc2)c2ncnn32)[C@@H](CN2C3CCC2COC3)CN1]

Compound 80 was synthesized as a white solid (45 mg) in a manner similar to that described in compound 174. ¹H NMR (400 MHz, DMSO-d⁶) δ (ppm) 8.50 (s, 1H), 8.42 (s, 1H), 7.39 (dd, J=8.2, 5.8 Hz, 2H), 7.11 (t, J=8.7 Hz, 2H), 4.30 (s, 2H), 4.23 (d, J=10.6 Hz, 1H), 4.12 (s, 2H), 4.01 (d, J=10.6 Hz, 1H), 3.90 (d, J=16.2 Hz, 1H), 3.43 (d, J=16.2 Hz, 1H), 2.85-2.77 (m, 1H), 2.77-2.67 (m, 2H), 2.65-2.52 (m, 3H), 2.48-2.41 (m, 1H), 2.30-2.18 (m, 2H), 2.06 (d, J=10.4 Hz, 1H), 1.93 (dd, J=18.6, 6.3 Hz, 2H), 1.72-1.62 (m, 2H), 1.58 (s, 6H), 1.55-1.51 (m, 1H), 1.49-1.39 (m, 1H), 0.87 (d, J=6.0 Hz, 3H). MS (ESI) m/z 562.5 [M+H]⁺.

Example 51: 1-(4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-((2-methyl-1H-imidazol-1-yl)methyl)piperazin-1-yl)ethan-1-one (Compound 91)

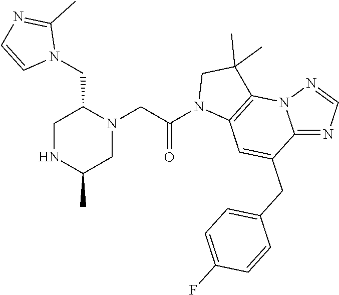 [SMILES: Cc1nccn1C[C@H]1CN[C@H](C)CN1CC(=O)N1CC(C)(C)c2c1cc(Cc1ccc(F)cc1)c1ncnn21]

Compound 91 was synthesized as a white solid (9 mg) in a manner similar to that described in compound 146. MS (ESI) m/z 531.4 [M+H]⁺.

Example 52: 2-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)isoindolin-1-one (Compound 94)

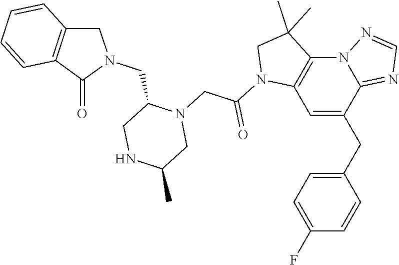 [SMILES: C[C@@H]1CN(CC(=O)N2CC(C)(C)c3c2cc(Cc2ccc(F)cc2)c2ncnn32)[C@@H](CN2Cc3ccccc3C2=O)CN1]

Following the synthesis of compound 207, compound 94 was synthesized as a white solid. MS (ESI) m/z 582.5 [M+H]⁺. ¹H NMR (400 MHz, DMSO-d⁶) δ (ppm) 8.48 (s, 1H), 8.00 (s, 1H), 7.49-7.37 (m, 4H), 7.27 (t, J=7.5 Hz, 1H), 7.22-7.12 (m, 2H), 7.06 (t, J=7.4 Hz, 1H), 4.63 (d, J=17.5 Hz, 1H), 4.41 (d, J=17.5 Hz, 1H), 4.27 (d, J=14.7 Hz, 1H), 4.15 (d, J=14.7 Hz, 1H), 3.99 (d, J=10.4 Hz, 1H), 3.92 (dd, J=15.2, 7.7 Hz, 1H), 3.82 (d, J=10.4 Hz, 1H), 3.70 (s, 2H), 3.32-3.23 (m, 3H), 2.82 (dd, J=11.8, 2.6 Hz, 1H), 2.77-2.61 (m, 2H), 2.60-2.55 (m, 1H), 2.44 (t, J=11.0 Hz, 1H), 1.57 (s, 3H), 1.45 (s, 3H), 0.91 (d, J=6.0 Hz, 3H).

Example 53: 6-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)-6,7-dihydro-5H-pyrrolo[3,4-b]pyridin-5-one (Compound 95)

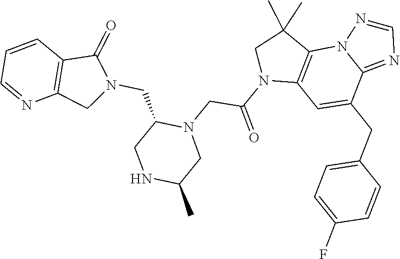 [SMILES: C[C@@H]1CN(CC(=O)N2CC(C)(C)c3c2cc(Cc2ccc(F)cc2)c2ncnn32)[C@@H](CN2Cc3ncccc3C2=O)CN1]

Following the synthesis of compound 207, compound 95 was synthesized as a white solid. MS (ESI) m/z 583.5 [M+H]⁺. ¹H NMR (400 MHz, DMSO-d⁶) δ (ppm) 8.56 (dd, J=5.0, 1.6 Hz, 1H), 8.50 (s, 1H), 7.94 (s, 1H), 7.85 (dd, J=7.6, 1.6 Hz, 1H), 7.46-7.37 (m, 2H), 7.20-7.13 (m, 2H), 7.11 (dd, J=7.7, 4.9 Hz, 1H), 4.68 (d, J=18.0 Hz, 1H), 4.52 (d, J=18.0 Hz, 1H), 4.31 (d, J=14.9 Hz, 1H), 4.12 (d, J=14.9 Hz, 1H), 4.11-3.85 (m, 3H), 3.79-3.64 (m, 2H), 3.32-3.21 (m, 3H), 2.92-2.84 (m, 1H), 2.77-2.66 (m, 2H), 2.51-2.43 (m, 2H), 1.61 (s, 3H), 1.49 (s, 3H), 0.93 (d, J=6.2 Hz, 3H).

Example 54: 2-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)-2,3-dihydro-1H-pyrrolo[3,4-c]pyridin-1-one (Compound 96)

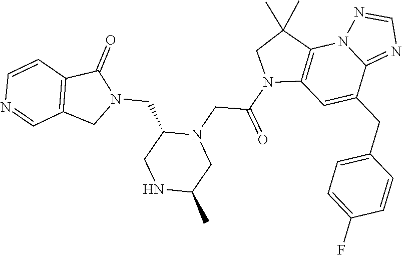 [SMILES: C[C@@H]1CN(CC(=O)N2CC(C)(C)c3c2cc(Cc2ccc(F)cc2)c2ncnn32)[C@@H](CN2Cc3cnccc3C2=O)CN1]

Following the synthesis of compound 207, compound 96 was synthesized as a white solid. MS (ESI) m/z 584.1 [M+H]⁺. H NMR (400 MHz, DMSO-d⁶) δ (ppm) 8.65 (s, 1H), 8.49 (s, 1H), 8.40 (d, J=5.0 Hz, 11H), 7.98 (s, 1H), 7.51 (d, J=5.1 Hz, 1H), 7.46-7.38 (m, 2H), 7.17 (t, J=8.8 Hz, 2H), 4.70 (d, J=18.7 Hz, 1H), 4.53 (d, J=18.7 Hz, 1H), 4.27 (d, J=14.7 Hz, 1H), 4.17 (d, J=14.7 Hz, 1H), 4.01 (d, J=10.4 Hz, 1H), 3.91 (dd, J=15.3, 7.5 Hz, 1H), 3.85 (d, J=10.5 Hz, 1H), 3.70 (s, 2H), 3.31-3.25 (m, 3H), 2.85 (dd, J=11.7, 2.6 Hz, 1H), 2.75-2.64 (m, 2H), 2.52-2.50 (m, 1H), 2.45 (t, J=11.0 Hz, 1H), 1.59 (s, 3H), 1.47 (s, 3H), 0.92 (d, J=5.9 Hz, 3H).

Example 55: 2-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)-1,2-dihydro-3H-pyrrolo[3,4-c]pyridin-3-one (Compound 97)

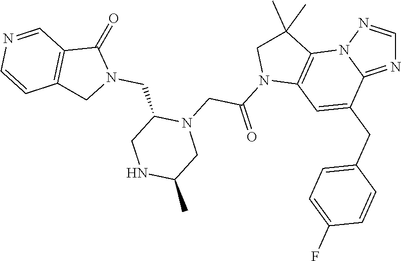 [SMILES: C[C@@H]1CN(CC(=O)N2CC(C)(C)c3c2cc(Cc2ccc(F)cc2)c2ncnn32)[C@@H](CN2Cc3ccncc3C2=O)CN1]

Following the synthesis of compound 207, compound 97 was synthesized as a white solid. MS (ESI) m/z 583.5 [M+H]⁺. 1H NMR (400 MHz, DMSO-d⁶) δ (ppm) 8.65 (s, 11H), 8.49 (d, J=2.1 Hz, 11H), 8.40 (dd, J=5.2, 2.4 Hz, 11H), 7.97 (s, 11H), 7.51 (t, J=3.8 Hz, 11H), 7.42 (dd, J=7.8, 5.0 Hz, 2H), 7.21-7.13 (m, 2H), 4.70 (d, J=18.7 Hz, 11H), 4.53 (d, J=18.7 Hz, 11H), 4.27 (d, J=14.5 Hz, 11H), 4.17 (d, J=14.8 Hz, 11H), 4.01 (d, J=10.5 Hz, 1H), 3.92 (d, J=9.4 Hz, 1H), 3.88-3.81 (m, 1H), 3.73-3.68 (m, 2H), 3.32-3.23 (m, 3H), 2.86 (s, 1H), 2.77-2.64 (m, 3H), 2.51-2.31 (m, 2H), 1.59 (s, 3H), 1.47 (s, 3H), 0.92 (d, J=6.2 Hz, 3H).

Example 56: 6-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)-5,6-dihydro-7H-pyrrolo[3,4-b]pyridin-7-one (Compound 98)

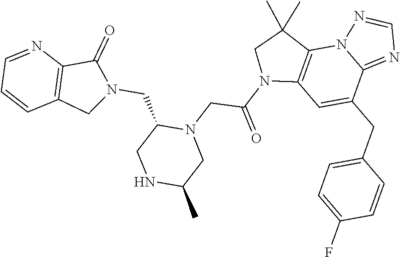 [SMILES: C[C@@H]1CN(CC(=O)N2CC(C)(C)c3c2cc(Cc2ccc(F)cc2)c2ncnn32)[C@@H](CN2Cc3cccnc3C2=O)CN1]

Following the synthesis of compound 207, compound 98 was synthesized as a white solid. MS (ESI) m/z 583.5 [M+H]⁺. ¹H NMR (400 MHz, DMSO-d⁶) δ (ppm) 8.49 (s, 1H), 8.10 (dd, J=4.8, 1.4 Hz, 1H), 7.91 (s, 1H), 7.78 (dd, J=7.7, 1.4 Hz, 1H), 7.44 (dd, J=8.6, 5.7 Hz, 2H), 7.23-7.14 (m, 2H), 7.06 (dd, J=7.7, 4.8 Hz, 1H), 4.62 (d, J=17.8 Hz, 1H), 4.41 (d, J=17.7 Hz, 1H), 4.26 (d, J=14.6 Hz, 1H), 4.15 (d, J=14.7 Hz, 1H), 4.00 (dd, J=14.5, 7.7 Hz, 1H), 3.94-3.84 (m, 2H), 3.80 (d, J=17.4 Hz, 1H), 3.62 (d, J=17.5 Hz, 1H), 3.33 (s, 2H), 3.23 (dd, J=14.6, 2.6 Hz, 1H), 2.84 (dd, J=11.7, 2.7 Hz, 1H), 2.71-2.60 (m, 3H), 2.44 (t, J=11.1 Hz, 1H), 1.66 (s, 3H), 1.44 (s, 3H), 0.92 (d, J=5.4 Hz, 3H).

Example 57: 4-fluoro-2-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)isoindolin-1-one (Compound 99)

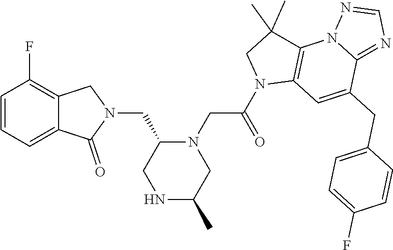 [SMILES: C[C@@H]1CN(CC(=O)N2CC(C)(C)c3c2cc(Cc2ccc(F)cc2)c2ncnn32)[C@@H](CN2Cc3c(F)cccc3C2=O)CN1]

Following the synthesis of compound 207, compound 99 was synthesized as a white solid. MS (ESI) m/z 601.1 [M+H]⁺. ¹H NMR (400 MHz, DMSO-d⁶) δ (ppm) 8.49 (s, 1H), 8.01 (d, J=8.9 Hz, 1H), 7.38 (dd, J=8.5, 5.7 Hz, 2H), 7.34 (d, J=7.2 Hz, 1H), 7.21 (t, J=8.6 Hz, 1H), 7.17-7.11 (m, 3H), 4.76-4.64 (m, 1H), 4.53 (d, J=17.6 Hz, 1H), 4.33 (d, J=14.9 Hz, 1H), 4.11 (d, J=14.9 Hz, 1H), 4.05-3.98 (m, 1H), 3.93 (dd, J=15.4, 7.6 Hz, 1H), 3.85 (d, J=10.4 Hz, 1H), 3.72 (d, J=10.2 Hz, 2H), 3.32-3.22 (m, 3H), 2.86 (d, J=11.4 Hz, 1H), 2.72 (d, J=11.7 Hz, 2H), 2.52-2.34 (m, 2H), 1.60 (s, 3H), 1.47 (s, 3H), 0.93 (d, J=5.9 Hz, 3H).

Example 58: 5-fluoro-2-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)isoindolin-1-one (Compound 100)

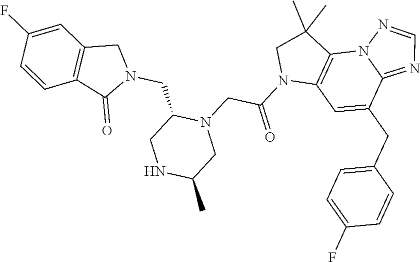 [SMILES: C[C@@H]1CN(CC(=O)N2CC(C)(C)c3c2cc(Cc2ccc(F)cc2)c2ncnn32)[C@@H](CN2Cc3cc(F)ccc3C2=O)CN1]

Following the synthesis of compound 207, compound 100 was synthesized as a white solid. MS (ESI) m/z 601.1 [M+H]⁺. ¹H NMR (400 MHz, DMSO-d⁶) δ (ppm) 8.49 (s, 1H), 8.00 (s, 1H), 7.47 (dd, J=8.4, 5.1 Hz, 1H), 7.43-7.34 (m, 3H), 7.20-7.10 (m, 2H), 6.86 (td, J=9.0, 2.3 Hz, 1H), 4.64 (d, J=17.9 Hz, 1H), 4.43 (d, J=17.8 Hz, 1H), 4.30 (d, J=14.6 Hz, 1H), 4.13 (d, J=14.7 Hz, 1H), 3.99 (d, J=10.4 Hz, 1H), 3.90 (dd, J=14.4, 6.9 Hz, 1H), 3.83 (d, J=10.4 Hz, 1H), 3.78-3.58 (m, 2H), 3.33-3.22 (m, 3H), 2.83 (dd, J=11.7, 2.5 Hz, 1H), 2.73-2.62 (m, 2H), 2.59-2.51 (m, 1H), 2.43 (t, J=11.1 Hz, 1H), 1.60 (s, 3H), 1.45 (s, 3H), 0.91 (d, J=6.0 Hz, 3H).

Example 59: 6-fluoro-2-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)isoindolin-1-one (Compound 101)

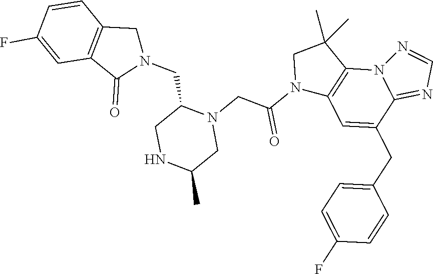 [SMILES: C[C@@H]1CN(CC(=O)N2CC(C)(C)c3c2cc(Cc2ccc(F)cc2)c2ncnn32)[C@@H](CN2Cc3ccc(F)cc3C2=O)CN1]

Following the synthesis of compound 207, compound 101 was synthesized as a white solid. MS (ESI) m/z 601.1 [M+H]⁺. ¹H NMR (400 MHz, DMSO-d⁶) δ (ppm) 8.49 (s, 1H), 7.98 (s, 1H), 7.50-7.38 (m, 2H), 7.25-7.07 (m, 4H), 4.61 (d, J=17.5 Hz, 1H), 4.41 (d, J=17.5 Hz, 1H), 4.29 (d, J=14.8 Hz, 1H), 4.15 (d, J=14.7 Hz, 1H), 4.00 (d, J=10.4 Hz, 1H), 3.91 (dd, J=15.2, 7.6 Hz, 1H), 3.84 (d, J=10.4 Hz, 1H), 3.69 (s, 2H), 3.32-3.24 (m, 3H), 2.82 (dd, J=11.8, 2.6 Hz, 1H), 2.73-2.62 (m, 2H), 2.43 (t, J=11.0 Hz, 1H), 2.12-1.97 (m, 1H), 1.61 (s, 3H), 1.46 (s, 3H), 0.91 (d, J=6.0 Hz, 3H).

Example 60: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrazolo[1,5-a]pyrrolo[2,3-e]pyridine-3-carbonitrile (Compound 105)

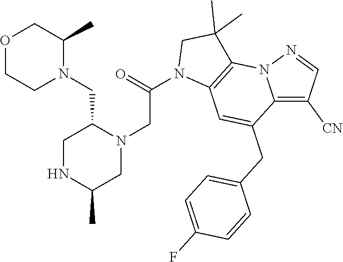 [SMILES: C[C@@H]1CN(CC(=O)N2CC(C)(C)c3c2cc(Cc2ccc(F)cc2)c2c(C#N)cnn32)[C@@H](CN2CCOC[C@H]2C)CN1]

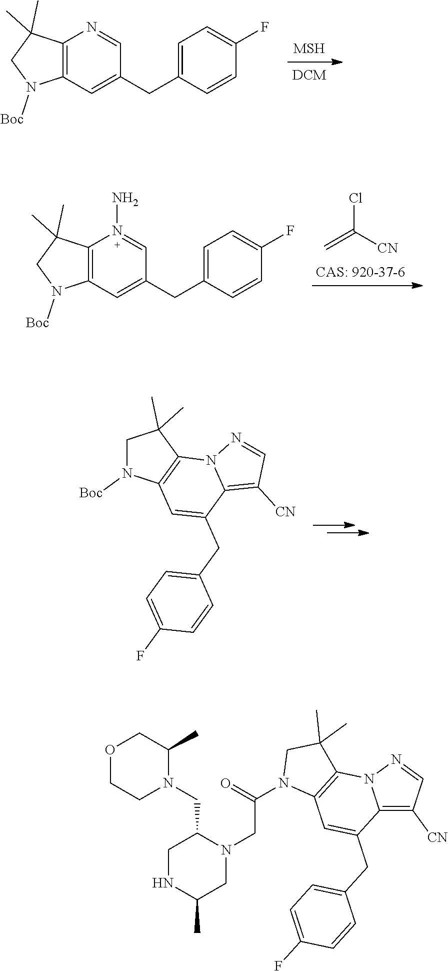

Step 1-2: tert-butyl 3-cyano-4-(4-fluorobenzyl)-8,8-dimethyl-7,8-dihydro-6H-pyrazolo[1,5-a]pyrrolo[2,3-e]pyridine-6-carboxylate

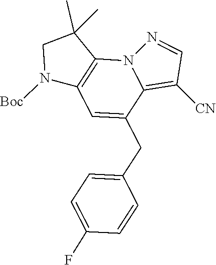 [SMILES: CC(C)(C)OC(=O)N1CC(C)(C)c2c1cc(Cc1ccc(F)cc1)c1c(C#N)cnn21]

To a solution of tert-butyl 6-(4-fluorobenzyl)-3,3-dimethyl-2,3-dihydro-1H-pyrrolo[3,2-b]pyridine-1-carboxylate (1.14 g, 3.2 mmol, 1.0 eq) in DCM (15 mL) was added the reagent MSH (2.41 g, 11.2 mmol, 3.5 eq) at 0° C. and the resulting solution was stirred at room temperature for 18 h. TLC showed the reaction was complete. The reaction solution was evaporated. The crude product was dissolved in DMF (30 mL) at rt, 2-chloroacrylonitrile (CAS: 920-37-6, 0.57 g, 6.4 mmol, 2.0 eq), K₂CO₃ (1.33 g, 9.6 mmol, 3.0 eq) were added sequentially and the resulting solution was stirred for 4 h. TLC showed reaction was complete. The mixture was diluted with 100 mL of H₂O and extracted with ethyl acetate and dried over sodium sulfate, then concentrated in vacuum and purified by silica gel flash column chromatography to give the title compound (0.05 g, 3.7% for two steps), LCMS: [M+H]⁺=421.2.

Following Steps: following the synthesis of compound 14, compound 105 was obtained as a white solid. MS (ESI) m/z 574.5[M+H]⁺. ¹H NMR (400 MHz, DMSO-d⁶) δ (ppm) 8.71 (s, 1H), 8.16 (s, 1H), 7.42-7.28 (m, 2H), 7.25-7.12 (m, 2H), 4.52-4.34 (m, 2H), 4.11 (d, J=10.6 Hz, 1H), 3.98-3.81 (m, 2H), 3.73 (d, J=17.3 Hz, 1H), 3.53-3.42 (m, 2H), 3.17-2.64 (m, 8H), 2.59-2.53 (m, 2H), 2.27-2.14 (m, 1H), 2.05-1.93 (m, 1H), 1.92-1.82 (m, 1H), 1.69-1.57 (m, 6H), 1.06 (d, J=6.1 Hz, 3H), 0.92 (d, J=6.2 Hz, 3H).

Example 61: 1-(3-chloro-4-(4-fluorobenzyl)-8,8-dimethyl-7,8-dihydro-6H-pyrazolo[1,5-a]pyrrolo[2,3-e]pyridin-6-yl)-2-((2R,5R)-5-methyl-2-(((R)-3-methylmorpholino)methyl) piperazin-1-yl)ethan-1-one (Compound 106)

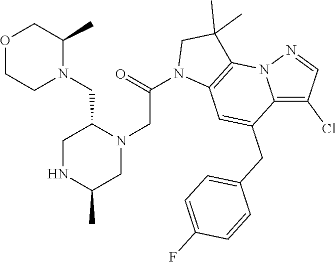 [SMILES: C[C@@H]1CN(CC(=O)N2CC(C)(C)c3c2cc(Cc2ccc(F)cc2)c2c(Cl)cnn32)[C@@H](CN2CCOC[C@H]2C)CN1]

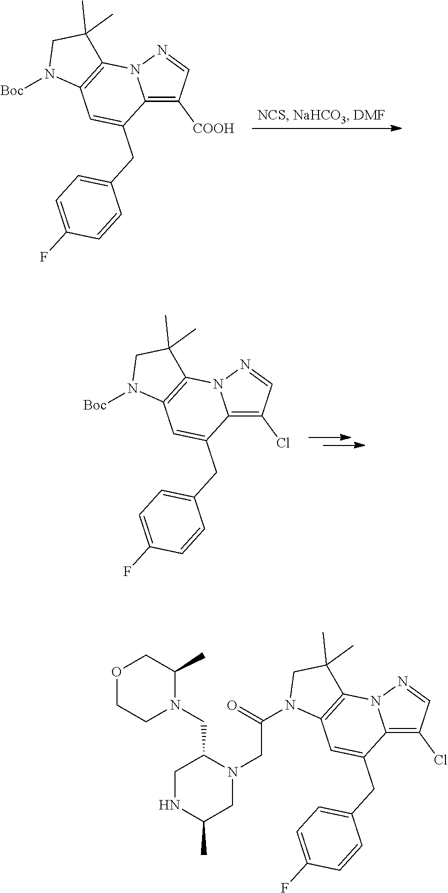

Step 1: tert-butyl 3-chloro-4-(4-fluorobenzyl)-8,8-dimethyl-7,8-dihydro-6H-pyrazolo[1,5-a]pyrrolo[2,3-e]pyridine-6-carboxylate

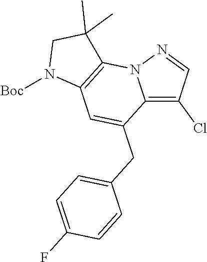 [SMILES: CC(C)(C)OC(=O)N1CC(C)(C)c2c1cc(Cc1ccc(F)cc1)c1c(Cl)cnn21]

At room temperature, sodium bicarbonate (27 mg, 0.327 mmol, 1.2 eq) and NCS (44 mg, 0.327 mmol, 1.2 eq) was added into the solution of 6-(tert-butoxycarbonyl)-4-(4-fluorobenzyl)-8,8-dimethyl-7,8-dihydro-6H-pyrazolo[1,5-a]pyrrolo[2,3-e]pyridine-3-carboxylic acid (120 mg, 0.27 mmol, 1.0 eq) in DMF (10 mL) and the resulting solution was stirred at room temperature for 18 h. TLC showed the reaction was complete. The mixture was diluted with 100 mL of H₂O and extracted with ethyl acetate and dried over sodium sulfate, then concentrated in vacuo and purified by silica gel flash column chromatography to get pure product (10 mg, 37%). LCMS: [M+H]⁺=430.2.

Following Steps: following the synthesis of compound 14, compound 106 was obtained as a white solid (18.1 mg). MS (ESI) m/z 583.5[M+H]⁺. ¹H NMR (400 MHz, DMSO-d⁶) δ (ppm) 8.12 (s, 1H), 8.01 (s, 1H), 7.32-7.21 (m, 2H), 7.20-7.08 (m, 2H), 4.54-4.36 (m, 2H), 4.10 (d, J=10.5 Hz, 1H), 3.91 (d, J=10.6 Hz, 1H), 3.81-3.61 (m, 2H), 3.54-3.44 (m, 2H), 3.21-3.13 (m, 1H), 3.07-2.97 (m, 1H), 2.95-2.73 (m, 4H), 2.71-2.60 (m, 2H), 2.58-2.52 (m, 1H), 2.37-2.27 (m, 1H), 2.23-2.11 (m, 1H), 1.98-1.88 (m, 1H), 1.77 (d, J=13.2 Hz, 1H), 1.61 (d, J=16.8 Hz, 6H), 0.93-0.88 (m, 6H).

Example 62: 1-(4-(4-fluorobenzyl)-1,2,8,8-tetramethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (Compound 109)

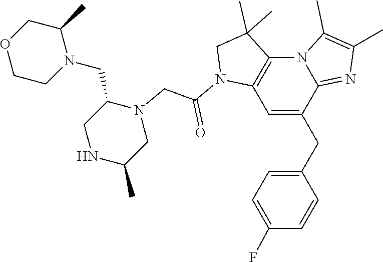 [SMILES: Cc1nc2c(Cc3ccc(F)cc3)cc3c(n2c1C)C(C)(C)CN3C(=O)CN1C[C@@H](C)NC[C@@H]1CN1CCOC[C@H]1C]

Compound 109 was synthesized as a white solid (20.8 mg) in a manner similar to that described in compound 116. ¹H NMR (400 MHz, DMSO-d⁶) δ (ppm) 8.00 (s, 1H), 7.35 (dd, J=8.5, 5.7 Hz, 2H), 7.09 (t, J=8.9 Hz, 2H), 4.20 (dd, J=31.7, 14.6 Hz, 2H), 4.05 (d, J=10.3 Hz, 1H), 3.90-3.77 (m, 2H), 3.56 (d, J=16.3 Hz, 1H), 3.50-3.42 (m, 2H), 3.20-3.13 (m, 1H), 2.96-2.77 (m, 4H), 2.71 (d, J=9.9 Hz, 1H), 2.65-2.56 (m, 5H), 2.42 (d, J=11.1 Hz, 1H), 2.33 (s, 3H), 2.27 (t, J=10.9 Hz, 1H), 2.18-2.08 (m, 1H), 1.92 (d, J=9.5 Hz, 1H), 1.74 (d, J=12.1 Hz, 1H), 1.53 (d, J=18.2 Hz, 6H), 0.92-0.85 (m, 6H). MS (ESI) m/z 577.4 [M+H]⁺.

Example 63: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-imidazo[1,2-a]pyrrolo[2,3-e]pyridine-1-carbonitrile (Compound 111)

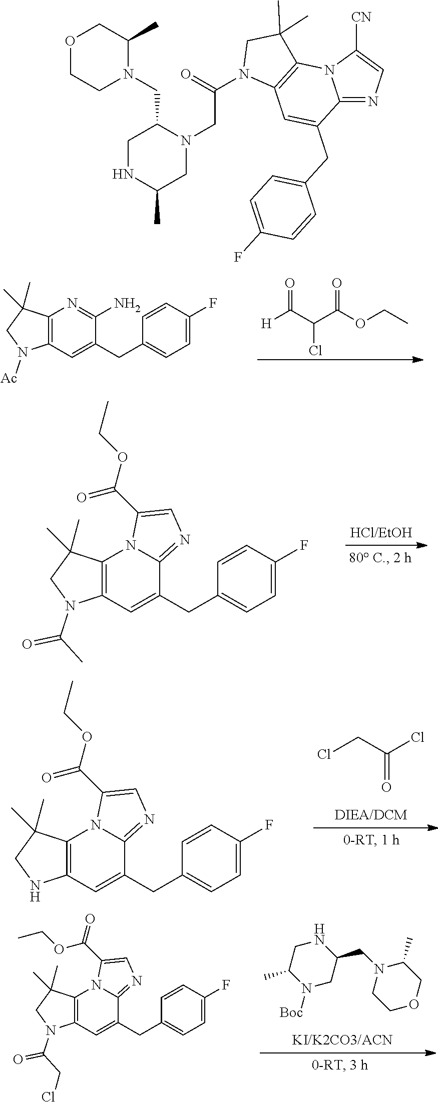

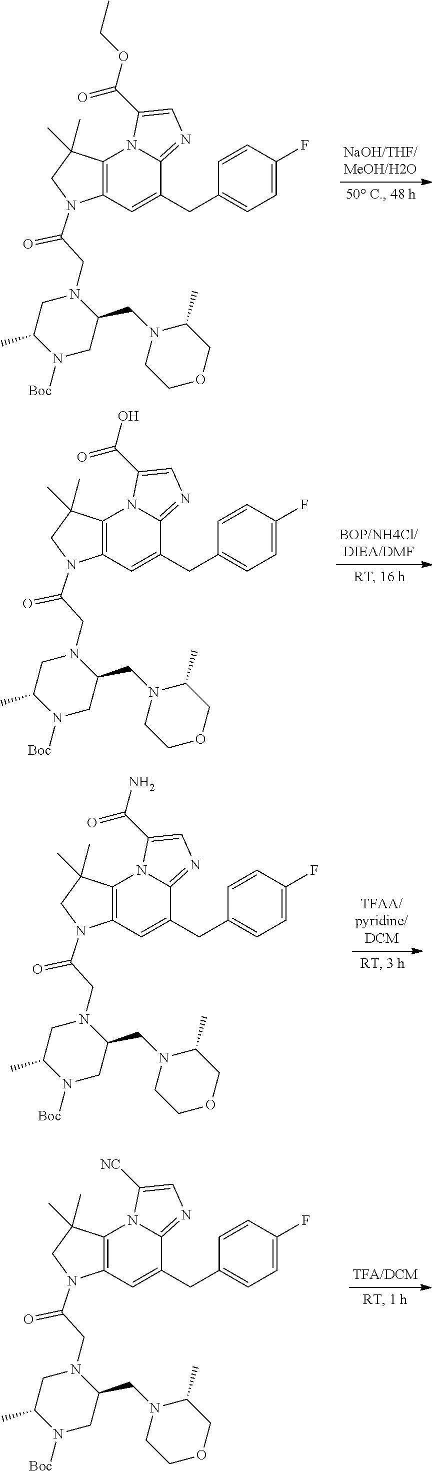 [SMILES: CCOC(=O)c1cnc2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C ; C[C@@H]1COCCN1C[C@H]1CN(C(=O)OC(C)(C)C)[C@H](C)CN1CC(=O)N1CC(C)(C)c2c1cc(Cc1ccc(F)cc1)c1ncc(C(=O)O)n21 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1ncc(C(N)=O)n21 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1ncc(C#N)n21]

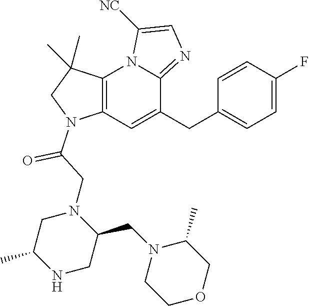 [SMILES: C[C@@H]1CN(CC(=O)N2CC(C)(C)c3c2cc(Cc2ccc(F)cc2)c2ncc(C#N)n32)[C@@H](CN2CCOC[C@H]2C)CN1]

Steps 1-4: ethyl 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-imidazo[1,2-a]pyrrolo[2,3-e]pyridine-1-carboxylate

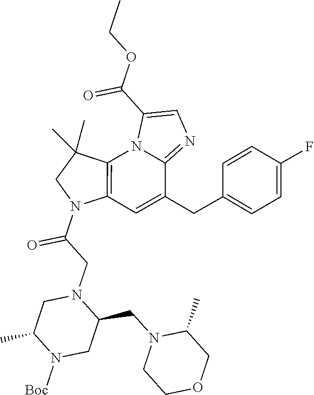 [SMILES: CCOC(=O)c1cnc2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C]

Following the synthesis of compound 116, steps 1-4 were performed to give the title compound (30 mg). MS (ESI) m/z 721.6 [M+H]⁺.

Step 5: 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-imidazo[1,2-a]pyrrolo[2,3-e]pyridine-1-carboxylic acid

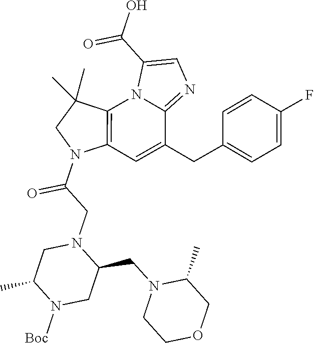 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1ncc(C(=O)O)n21]

Ethyl 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-imidazo[1,2-a]pyrrolo[2,3-e]pyridine-1-carboxylate (0.03 g, 0.042 mmol, 1.0 eq) and NaOH (0.017 g, 0.416 mmol, 10 eq) were dissolved in water (2 mL), THF (2.0 mL), and MeOH (2.0 mL) under argon to give a yellow solution. The reaction mixture was stirred at 50° C. for 48 h. 1M HCl (10 mL) was added to the reaction mixture followed by extraction with ethyl acetate (30 mL×2). The combined organic layers were dried over Na₂SO₄, filtered and concentrated to give the product (0.029 g, 100%). MS (ESI) m/z 693.5 [M+H]⁺.

Step 6: tert-butyl (2R,5S)-4-(2-(1-carbamoyl-4-(4-fluorobenzyl)-8,8-dimethyl-7,8-dihydro-6H-imidazo[1,2-a]pyrrolo[2,3-e]pyridin-6-yl)-2-oxoethyl)-2-methyl-5-(((R)-3-methylmorpholino)methyl)piperazine-1-carboxylate

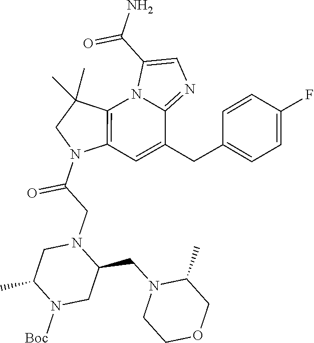 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1ncc(C(N)=O)n21]

6-(2-((2S,5R)-4-(tert-butoxycarbonyl)-5-methyl-2-(((R)-3-methylmorpholino)meth yl)piperazin-1-yl)acetyl)-4-(4-fluorobenzyl)-8,8-dimethyl-7,8-dihydro-6H-imidazo[1,2-a]pyrrolo[2,3-e]pyridine-1-carboxylic acid (0.029 g, 0.042 mmol, 1.0 eq), BOP (0.037 g, 0.084 mmol, 2.0 eq), DIEA (0.081 g, 0.628 mmol, 15 eq), and NH₄Cl (0.022 g, 0.419 mmol, 10 eq) were dissolved in DMF (5 mL) under argon to give a yellow solution. The reaction mixture was stirred at rt for 12 h. H₂O (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 8% to give the product (0.016 g, 55.3%) as a yellow solid. MS (ESI) m/z 692.5 [M+H]⁺.

Steps 7-8: Following the synthesis of compound 153, compound 111 was obtained as a white solid (3.6 mg). ¹H NMR (400 MHz, DMSO-d⁶) δ (ppm) 8.57 (s, 1H), 8.44 (s, 1H), 7.38 (dd, J=8.5, 5.7 Hz, 2H), 7.11 (t, J=8.9 Hz, 2H), 4.34 (q, J=14.6 Hz, 2H), 4.16 (d, J=10.5 Hz, 1H), 3.93 (d, J=10.6 Hz, 1H), 3.81 (d, J=16.6 Hz, 1H), 3.66 (d, J=16.7 Hz, 1H), 3.49-3.44 (m, 2H), 3.15-3.09 (m, 1H), 3.00-2.94 (m, 1H), 2.92-2.79 (m, 3H), 2.75-2.68 (m, 1H), 2.61 (d, J=8.6 Hz, 2H), 2.47-2.42 (m, 1H), 2.35-2.24 (m, 1H), 2.20-2.10 (m, 1H), 1.97-1.87 (m, 1H), 1.75 (d, J=12.9 Hz, 1H), 1.68 (d, J=12.7 Hz, 6H), 0.91-0.86 (m, 6H). MS (ESI) m/z 574.6 [M+H]⁺.

Example 64: 1-(2-chloro-4-(4-fluorobenzyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (Compound 113)

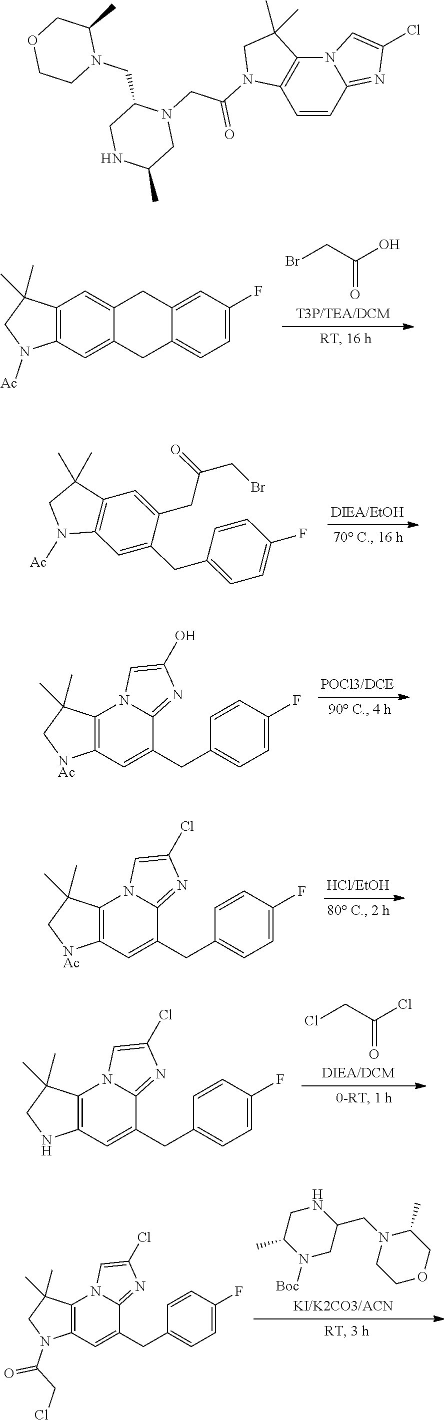

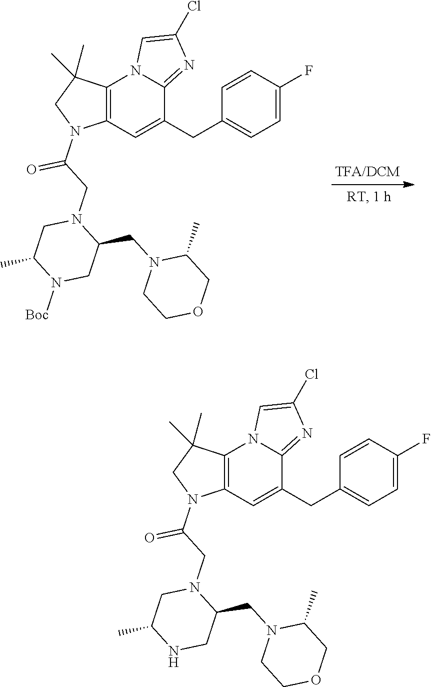 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(Cl)cn21 ; C[C@@H]1CN(CC(=O)N2CC(C)(C)c3c2cc(Cc2ccc(F)cc2)c2nc(Cl)cn32)[C@@H](CN2CCOC[C@H]2C)CN1]

Step 1: N-(1-acetyl-6-(4-fluorobenzyl)-3,3-dimethyl-2,3-dihydro-1H-pyrrolo[3,2-b]pyridin-5-yl)-2-bromoacetamide

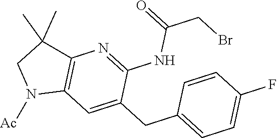 [SMILES: CC(=O)N1CC(C)(C)c2nc(NC(=O)CBr)c(Cc3ccc(F)cc3)cc21]

1-(5-amino-6-(4-fluorobenzyl)-3,3-dimethyl-2,3-dihydro-1H-pyrrolo[3,2-b]pyridin-1-yl)ethan-1-one (0.06 g, 0.191 mmol, 1.0 eq), 2-bromoacetic acid (0.040 g, 0.287 mmol, 1.5 eq), TEA (0.058 g, 0.574 mmol, 3.0 eq), and T₃P (0.244 g, 0.383 mmol, 2.0 eq) were dissolved in DCM (10 mL) under argon to give a yellow solution. The reaction mixture was stirred at rt for 16 h. Saturated NaCl (20 mL) was added to the reaction mixture followed by extraction with dichloromethane (30 mL×2). The combined organic layers were dried over Na₂SO₄, filtered and concentrated. The crude product was added to a silica gel column and was eluted with ethyl acetate/hexane from 0% to 80% to give the product (0.05 g, 60.1%) as a yellow oil. MS (ESI) m/z 434.2 [M+H]⁺.

Step 2: 1-(4-(4-fluorobenzyl)-2-hydroxy-8,8-dimethyl-7,8-dihydro-6H-imidazo[1,2-a]pyrrolo[2,3-e]pyridin-6-yl)ethan-1-one

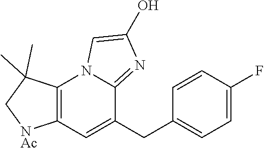

N-(1-acetyl-6-(4-fluorobenzyl)-3,3-dimethyl-2,3-dihydro-1H-pyrrolo[3,2-b]pyridin-5-yl)-2-bromoacetamide (0.05 g, 0.115 mmol, 1.0 eq) and DIEA (0.015 g, 0.115 mmol, 1.0 eq) were dissolved in EtOH (5 mL) under argon to give a yellow solution. The reaction mixture was stirred at 70° C. for 16 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.0256 g, 62.9%) as a yellow solid. MS (ESI) m/z 354.4 [M+H]⁺.

Step 3: 1-(2-chloro-4-(4-fluorobenzyl)-8,8-dimethyl-7,8-dihydro-6H-imidazo[1,2-a]pyrrolo[2,3-e]pyridin-6-yl)ethan-1-one

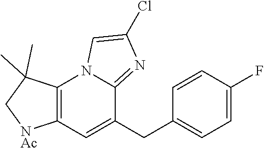

1-(4-(4-fluorobenzyl)-2-hydroxy-8,8-dimethyl-7,8-dihydro-6H-imidazo[1,2-a]pyrrolo[2,3-e]pyridin-6-yl)ethan-1-one (0.0256 g, 0.072 mmol, 1.0 eq) and POCl₃ (0.111 g, 0.724 mmol, 10 eq) were dissolved in 1,2-dichloroethane (10 mL) under argon to give a solution. The reaction mixture was stirred at 90° C. for 3 h. Saturated NaHCO₃ (30 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 50% to give the product (0.02 g, 74.3%) as a yellow oil. MS (ESI) m/z 372.4 [M+H]⁺.

Steps 4-7: Following the synthesis of compound 14, compound 113 was obtained as a white solid (6.9 mg). ¹H NMR (400 MHz, DMSO-d⁶) δ (ppm) 8.26 (s, 1H), 8.11 (s, 1H), 7.36 (dd, J=8.4, 5.7 Hz, 2H), 7.12 (t, J=8.9 Hz, 2H), 4.21 (q, J=14.6 Hz, 2H), 4.07 (d, J=10.5 Hz, 1H), 3.86 (d, J=10.6 Hz, 1H), 3.76 (d, J=16.9 Hz, 1H), 3.64 (d, J=16.5 Hz, 1H), 3.48-3.43 (m, 2H), 3.13 (t, J=9.4 Hz, 1H), 3.06-2.98 (m, 1H), 2.89-2.71 (m, 3H), 2.70-2.57 (m, 3H), 2.34-2.28 (m, 2H), 2.20-2.09 (m, 1H), 1.96-1.86 (m, 1H), 1.75 (d, J=12.1 Hz, 1H), 1.51 (d, J=16.5 Hz, 6H), 0.92-0.87 (m, 6H). MS (ESI) m/z 583.5 [M+H]⁺.

Example 65: 1-(4-(4-fluoro-2-methylbenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 115)

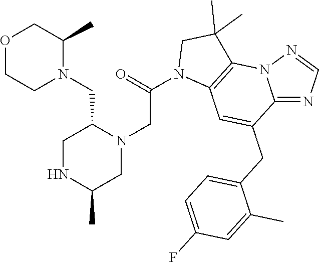 [SMILES: Cc1cc(F)ccc1Cc1cc2c(n3ncnc13)C(C)(C)CN2C(=O)CN1C[C@@H](C)NC[C@@H]1CN1CCOC[C@H]1C]

Compound 115 was synthesized in a manner similar to that described in compound 14. MS (ESI) m/z 564.5[M+H]⁺. ¹H NMR (400 MHz, DMSO-d⁶) δ (ppm) 8.54 (s, 1H), 8.19 (s, 1H), 7.33-7.21 (m, 1H), 7.15-7.06 (m, 1H), 7.05-6.93 (m, 1H), 4.41-4.24 (m, 2H), 4.17 (d, J=10.6 Hz, 1H), 3.97 (d, J=10.6 Hz, 1H), 3.81 (d, J=16.7 Hz, 1H), 3.67 (d, J=16.6 Hz, 1H), 3.52-3.46 (m, 2H), 3.19-3.08 (m, 1H), 3.06-2.95 (m, 1H), 2.94-2.72 (m, 4H), 2.69-2.58 (m, 2H), 2.51-2.43 (m, 1H), 2.37-2.25 (m, 4H), 2.22-2.11 (m, 1H), 2.04-1.89 (m, 1H), 1.77 (d, J=12.0 Hz, 1H), 1.63 (d, J=17.6 Hz, 6H), 0.96-0.87 (m, 6H).

Example 66: 1-(4-(4-fluorobenzyl)-8,8-dimethyl-2-(trifluoromethyl)-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (Compound 116)

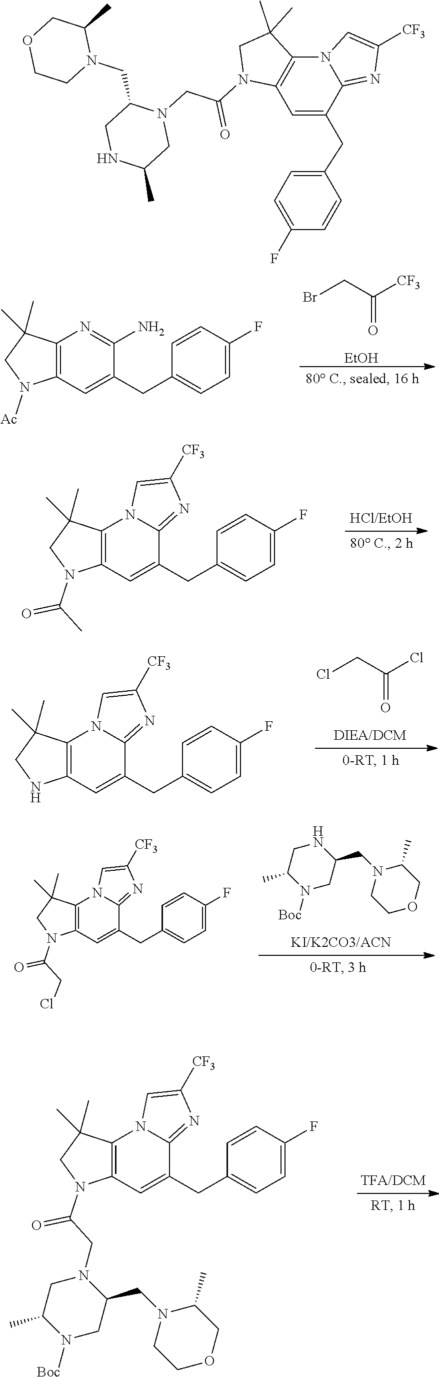

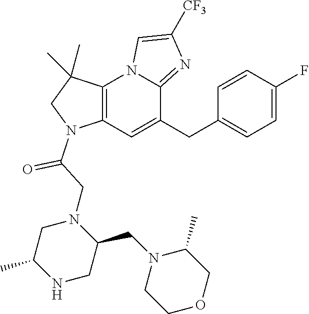 [SMILES: C[C@@H]1CN(CC(=O)N2CC(C)(C)c3c2cc(Cc2ccc(F)cc2)c2nc(C(F)(F)F)cn32)[C@@H](CN2CCOC[C@H]2C)CN1]

Step 1: 1-(4-(4-fluorobenzyl)-8,8-dimethyl-2-(trifluoromethyl)-7,8-dihydro-6H-imidazo[1,2-a]pyrrolo[2,3-e]pyridin-6-yl)ethan-1-one

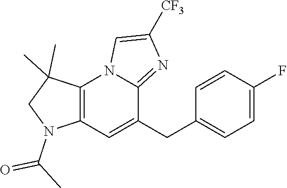 [SMILES: CC(=O)N1CC(C)(C)c2c1cc(Cc1ccc(F)cc1)c1nc(C(F)(F)F)cn21]

1-(5-amino-6-(4-fluorobenzyl)-3,3-dimethyl-2,3-dihydro-1H-pyrrolo[3,2-b]pyridin-1-yl)ethan-1-one (0.09 g, 0.287 mmol, 1.0 eq) and 3-bromo-1,1,1-trifluoropropan-2-one (0.274 g, 1.436 mmol, 5.0 eq) were dissolved in EtOH (5 mL) under argon to give a yellow solution. The reaction mixture was stirred at 80° C. for 16 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.095 g, 82%) as a yellow oil. MS (ESI) m/z 406.3 [M+H]⁺.

Steps 2-5: Following the synthesis of compound 14, compound 116 was obtained as a white solid (26.1 mg). ¹H NMR (400 MHz, DMSO-d⁶) δ (ppm) 8.70 (s, 1H), 8.19 (s, 1H), 7.38 (dd, J=8.5, 5.7 Hz, 2H), 7.13 (t, J=8.9 Hz, 2H), 4.28 (q, J=15.0 Hz, 2H), 4.10 (d, J=10.6 Hz, 1H), 3.88 (d, J=10.6 Hz, 1H), 3.78 (d, J=16.6 Hz, 1H), 3.63 (d, J=16.6 Hz, 1H), 3.50-3.42 (m, 2H), 3.13 (t, J=9.9 Hz, 1H), 2.97 (t, J=8.7 Hz, 1H), 2.91-2.76 (m, 3H), 2.69 (dd, J=11.6, 2.2 Hz, 1H), 2.65-2.55 (m, 2H), 2.45 (t, J=11.0 Hz, 1H), 2.27 (t, J=11.0 Hz, 1H), 2.19-2.08 (m, 1H), 1.90 (t, J=9.4 Hz, 1H), 1.73 (d, J=12.4 Hz, 1H), 1.55 (d, J=17.3 Hz, 6H), 0.95-0.82 (m, 6H). MS (ESI) m/z 617.6 [M+H]⁺.

Example 67: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-imidazo[1,2-a]pyrrolo[2,3-e]pyridine-2-carboxylic acid (Compound 117)

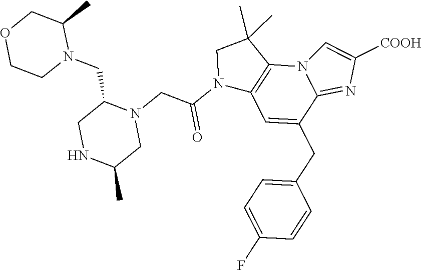 [SMILES: C[C@@H]1CN(CC(=O)N2CC(C)(C)c3c2cc(Cc2ccc(F)cc2)c2nc(C(=O)O)cn32)[C@@H](CN2CCOC[C@H]2C)CN1]

Compound 117 was obtained as a white solid (2.5 mg) after hydrolysis of compound 18. ¹H NMR (400 MHz, DMSO-d⁶) δ (ppm) 8.31 (s, 1H), 8.02 (s, 1H), 7.37 (s, 2H), 7.12 (t, J=8.3 Hz, 2H), 4.29 (q, J=15.2 Hz, 2H), 4.04 (s, 1H), 3.86 (d, J=10.6 Hz, 1H), 3.76-3.65 (m, 4H), 3.12-3.10 (m, 1H), 2.89-2.79 (m, 4H), 2.78-2.73 (m, 2H), 2.68-2.60 (m, 2H), 2.43-2.34 (m, 1H), 2.20-2.09 (m, 1H), 1.92 (t, J=9.0 Hz, 1H), 1.78 (d, J=13.2 Hz, 1H), 1.53 (d, J=16.5 Hz, 6H), 0.92 (dd, J=27.2, 6.1 Hz, 6H). MS (ESI) m/z 593.5 [M+H]⁺.

Example 68: 1-(1-chloro-4-(4-fluorobenzyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (Compound 118)

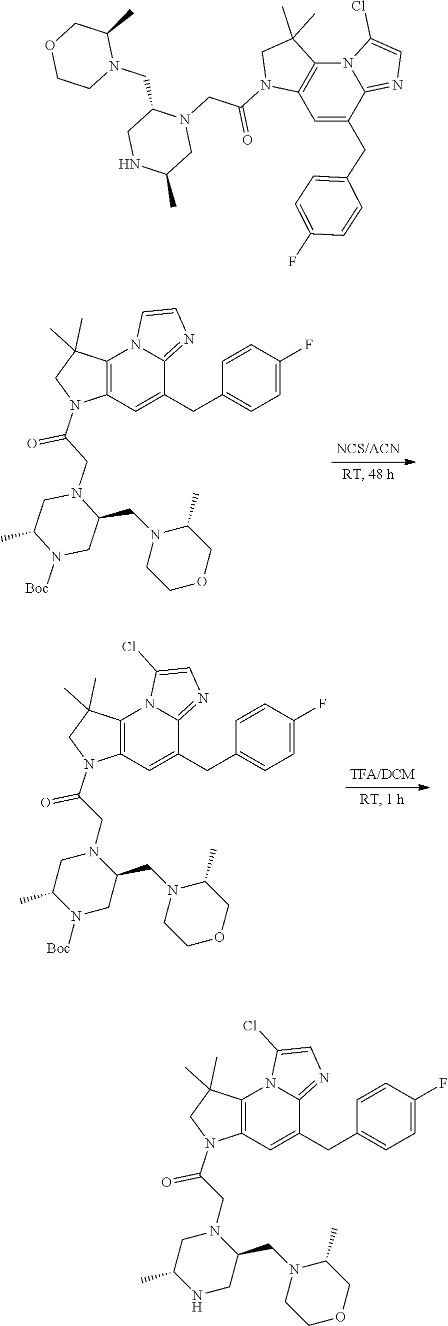 [SMILES: C[C@@H]1CN(CC(=O)N2CC(C)(C)c3c2cc(Cc2ccc(F)cc2)c2ncc(Cl)n32)[C@@H](CN2CCOC[C@H]2C)CN1 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nccn21 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1ncc(Cl)n21 ; C[C@@H]1CN(CC(=O)N2CC(C)(C)c3c2cc(Cc2ccc(F)cc2)c2ncc(Cl)n32)[C@@H](CN2CCOC[C@H]2C)CN1]

Step 1: tert-butyl (2R,5S)-4-(2-(1-chloro-4-(4-fluorobenzyl)-8,8-dimethyl-7,8-dihydro-6H-imidazo[1,2-a]pyrrolo[2,3-e]pyridin-6-yl)-2-oxoethyl)-2-methyl-5-(((R)-3-methylmorpholino)methyl)piperazine-1-carboxylate

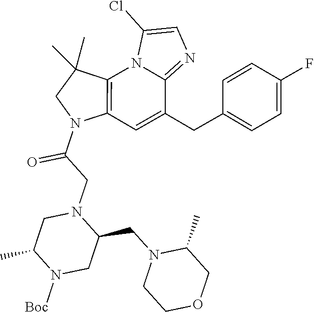 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1ncc(Cl)n21]

Tert-butyl (2R,5S)-4-(2-(4-(4-fluorobenzyl)-8,8-dimethyl-7,8-dihydro-6H-imidazo[1,2-a]pyrrolo[2,3-e]pyridin-6-yl)-2-oxoethyl)-2-methyl-5-(((R)-3-methylmorpholino)methyl)piperazine-1-carboxylate (0.02 g, 0.031 mmol, 1.0 eq) and NCS (4.12 mg, 0.031 mmol, 1.0 eq) were dissolved in acetonitrile (5 mL) under argon to give a yellow solution. The reaction mixture was stirred at rt for 48 h. H₂O (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.017 g, 81%) as a yellow oil. MS (ESI) m/z 683.5 [M+H]⁺.

Step 2: 1-(1-chloro-4-(4-fluorobenzyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (compound 118)

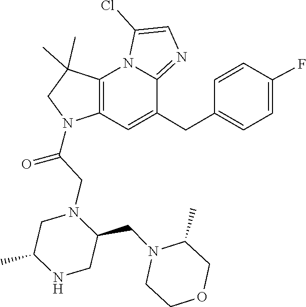 [SMILES: C[C@@H]1CN(CC(=O)N2CC(C)(C)c3c2cc(Cc2ccc(F)cc2)c2ncc(Cl)n32)[C@@H](CN2CCOC[C@H]2C)CN1]

Above obtained compound (0.017 g, 0.025 mmol, 1.0 eq) was dissolved in TFA (1 mL) and DCM (3.00 mL) under argon to give a yellow solution. The reaction mixture was stirred at rt for 2 h. Saturated NaHCO₃ (30 mL) was added to the reaction mixture followed by extraction with ethyl acetate (30 mL×2). The combined organic layers were dried over Na₂SO₄, filtered and concentrated. The crude product was purified by column chromatography to give the product (5.1 mg, 35.2%) as a white solid. ¹H NMR (400 MHz, DMSO-d⁶) δ (ppm) 8.13 (s, 1H), 7.74 (s, 1H), 7.35 (dd, J=8.2, 5.8 Hz, 2H), 7.12 (t, J=8.8 Hz, 2H), 4.26 (q, J=14.9 Hz, 2H), 4.06-3.80 (m, 3H), 3.68-3.46 (m, 3H), 3.25-3.01 (m, 4H), 3.01-2.78 (m, 3H), 2.73 (d, J=10.6 Hz, 1H), 2.69-2.57 (m, 1H), 2.27-2.12 (m, 1H), 2.09-1.82 (m, 3H), 1.69 (d, J=12.3 Hz, 6H), 1.15 (d, J=4.6 Hz, 3H), 0.91 (d, J=5.7 Hz, 3H). MS (ESI) m/z 583.8 [M+H]⁺.

Example 69: 1-(1-bromo-4-(4-fluorobenzyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (Compound 119)

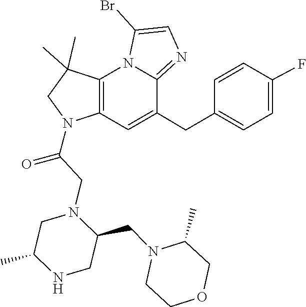 [SMILES: C[C@@H]1CN(CC(=O)N2CC(C)(C)c3c2cc(Cc2ccc(F)cc2)c2ncc(Br)n32)[C@@H](CN2CCOC[C@H]2C)CN1]

Compound 119 was synthesized as a white solid (7.7 mg) in a manner similar to that described in compound 118. H NMR (400 MHz, DMSO-d⁶) δ (ppm) 8.22 (s, 1H), 7.72 (s, 1H), 7.37 (dd, J=8.3, 5.8 Hz, 2H), 7.10 (t, J=8.8 Hz, 2H), 4.25 (q, J=14.5 Hz, 2H), 4.10 (d, J=10.4 Hz, 1H), 3.91-3.77 (m, 2H), 3.62 (d, J=16.4 Hz, 1H), 3.49-3.43 (m, 2H), 3.15 (t, J=10.3 Hz, 1H), 2.97-2.76 (m, 4H), 2.71 (d, J=9.9 Hz, 1H), 2.60 (d, J=8.9 Hz, 2H), 2.43 (d, J=10.8 Hz, 1H), 2.32-2.23 (m, 1H), 2.19-2.07 (m, 1H), 2.04-1.86 (m, 2H), 1.74 (d, J=13.1 Hz, 6H), 0.96-0.86 (m, 6H). MS (ESI) m/z 626.6 [M+H]⁺.

Example 70: ethyl 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrazolo[1,5-a]pyrrolo[2,3-e]pyridine-3-carboxylate (Compound 120)

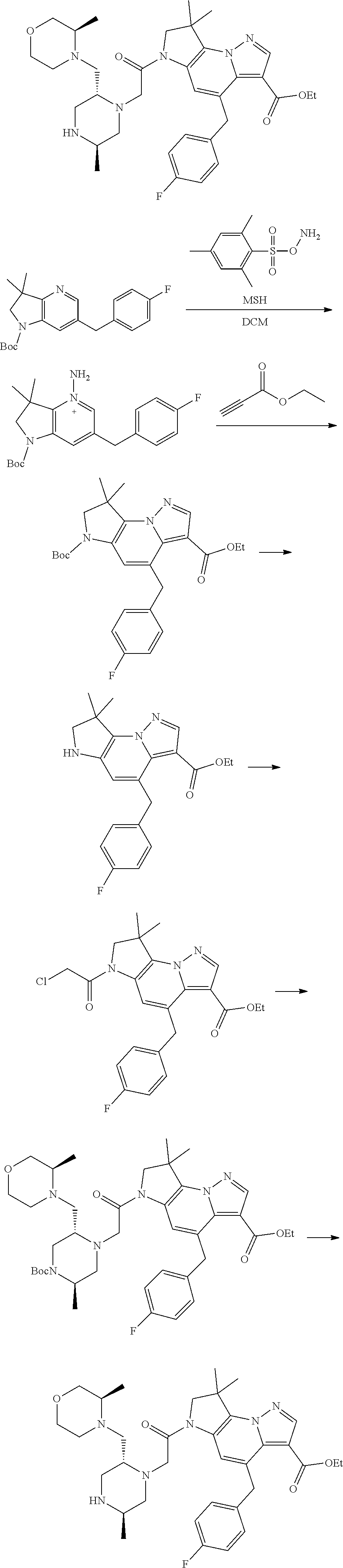

Steps 1-2: 6-(tert-butyl) 3-ethyl 4-(4-fluorobenzyl)-8,8-dimethyl-7,8-dihydro-6H-pyrazolo[1,5-a]pyrrolo[2,3-e]pyridine-3,6-dicarboxylate

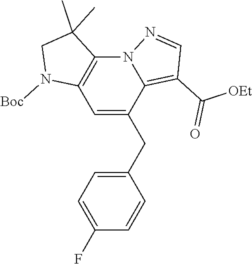 [SMILES: CCOC(=O)c1cnn2c3c(cc(Cc4ccc(F)cc4)c12)N(C(=O)OC(C)(C)C)CC3(C)C]

To a solution of tert-butyl 6-(4-fluorobenzyl)-3,3-dimethyl-2,3-dihydro-1H-pyrrolo[3,2-b]pyridine-1-carboxylate (1.14 g, 3.2 mmol, 1.0 eq) in DCM (15 mL) was added the reagent MSH (2.41 g, 11.2 mmol, 3.5 eq) at 0° C. and the resulting solution was stirred at room temperature for 18 h. TLC showed the reaction was complete. The reaction solution was evaporated. The crude product was dissolved in DMF (30 mL) at room temperature. Ethyl propiolate (0.63 g, 6.4 mmol, 2.0 eq), K₂CO₃ (1.33 g, 9.6 mmol, 3.0 eq) were added sequentially and the resulting solution was stirred for 4 h. TLC showed reaction was complete. The mixture was diluted with 100 mL of H₂O and extracted with ethyl acetate and dried over sodium sulfate, then concentrated in vacuum and purified by silica gel flash column chromatography to give the title compound (0.25 g, 16.8% for two steps). LCMS: [M+H]⁺=468.1.

Step 3: ethyl 4-(4-fluorobenzyl)-8,8-dimethyl-7,8-dihydro-6H-pyrazolo[1,5-a]pyrrolo[2,3-e]pyridine-3-carboxylate

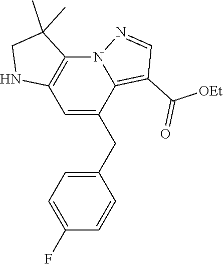 [SMILES: CCOC(=O)c1cnn2c3c(cc(Cc4ccc(F)cc4)c12)NCC3(C)C]

Above intermediate (50 mg, 0.107 mmol) in hydrogen chloride (4M solution in dioxane) was stirred for 1 h at 0° C. The solvent was removed to afford the title compound (50 mg, crude), LCMS: [M+H]⁺=368.2.

Step 4: ethyl 6-(2-chloroacetyl)-4-(4-fluorobenzyl)-8,8-dimethyl-7,8-dihydro-6H-pyrazolo[1,5-a]pyrrolo[2,3-e]pyridine-3-carboxylate

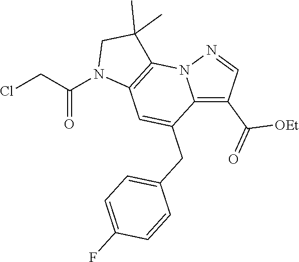 [SMILES: CCOC(=O)c1cnn2c3c(cc(Cc4ccc(F)cc4)c12)N(C(=O)CCl)CC3(C)C]

To a solution of above intermediate (50 mg, crude) in DCM was added DIPEA (2.0 eq) at 0° C. After stirring for 1.5 h, water added to the residue, and the mixture was extracted with DCM. The solvent was removed in vacuum to give a crude product, which is directly used in the next step.

Steps 5-6: ethyl 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrazolo[1,5-a]pyrrolo[2,3-e]pyridine-3-carboxylate (compound 120)

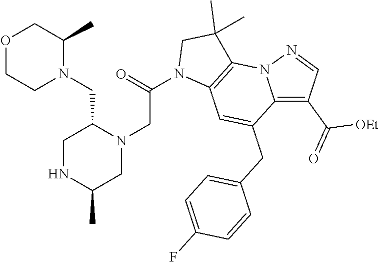 [SMILES: CCOC(=O)c1cnn2c3c(cc(Cc4ccc(F)cc4)c12)N(C(=O)CN1C[C@@H](C)NC[C@@H]1CN1CCOC[C@H]1C)CC3(C)C]

A mixture of K₂CO₃ (2.0 eq.), KI (1.5 eq.) and tert-butyl (2R,5S)-2-methyl-5-(((R)-3-methylmorpholino)methyl)piperazine-1-carboxylate II (1.2 eq.) was added to a solution of above crude product at 25° C. The reaction was stirred at R.T. for 1 h. LC-MS indicated the starting material was consumed completely and new peak formed. ([M+H]⁺=721.23). The reaction mixture was quenched by addition of water, then extracted with ethyl acetate, washed with brine, dried over Na₂SO₄ and concentrated under reduced pressure to give the key intermediate. Then the residue was dissolved in 4M HCl/dioxane. The mixture was stirred at R.T. for 1 h. HPLC-MS indicated a new main peak with desired MS formed, then quenched with water and purified by pre-HPLC (acetonitrile 20% to 50% in 30 min, 45 mL/min). The desired product was obtained as a white solid after lyophilization (2 mg). MS (ESI) m/z 621.6[M+H]+.

Example 71: 1-(4-(4-fluorobenzyl)-2-(fluoromethyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (Compound 121)

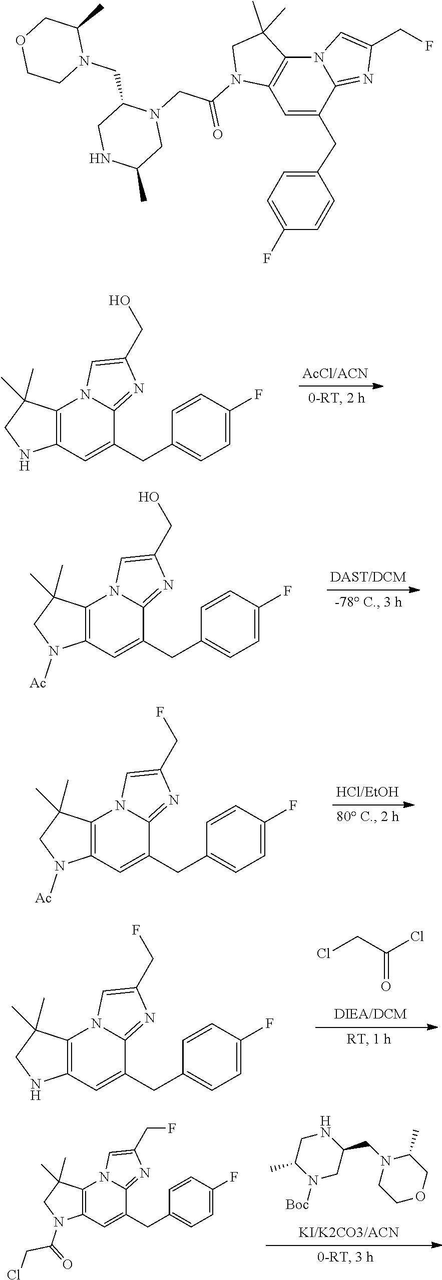

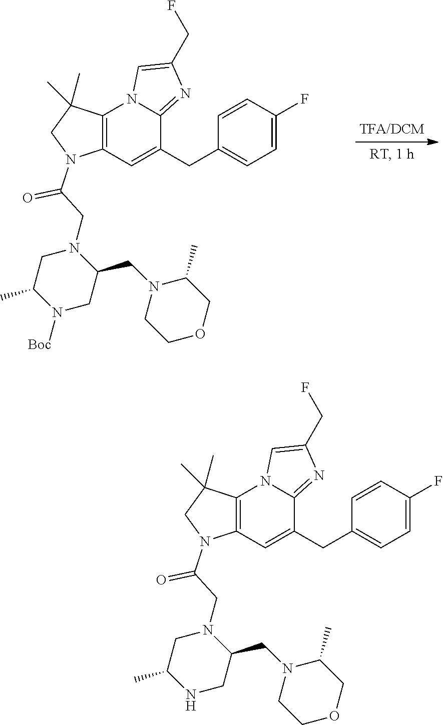 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(CF)cn21 ; C[C@@H]1CN(CC(=O)N2CC(C)(C)c3c2cc(Cc2ccc(F)cc2)c2nc(CF)cn32)[C@@H](CN2CCOC[C@H]2C)CN1]

Step 1: 1-(4-(4-fluorobenzyl)-2-(hydroxymethyl)-8,8-dimethyl-7,8-dihydro-6H-imidazo[1,2-a]pyrrolo[2,3-e]pyridin-6-yl)ethan-1-one

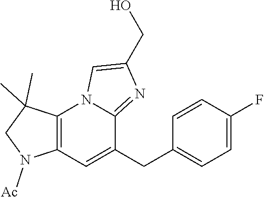 [SMILES: CC(=O)N1CC(C)(C)c2c1cc(Cc1ccc(F)cc1)c1nc(CO)cn21]

(4-(4-fluorobenzyl)-8,8-dimethyl-7,8-dihydro-6H-imidazo[1,2-a]pyrrolo[2,3-e]pyri din-2-yl)methanol (0.03 g, 0.092 mmol, 1.0 eq) and AcCl (0.014 g, 0.184 mmol, 2.0 eq) were dissolved in acetonitrile (5 mL) under argon to give a yellow solution. The reaction mixture was stirred at rt for 2 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.03 g, 89%) as a yellow oil. MS (ESI) m/z 368.4 [M+H]⁺.

Step 2

1-(4-(4-fluorobenzyl)-2-(fluoromethyl)-8,8-dimethyl-7,8-dihydro-6H-imidazo[1,2-a]pyrrolo[2,3-e]pyridin-6-yl)ethan-1-one

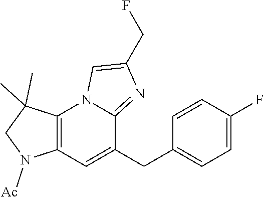 [SMILES: CC(=O)N1CC(C)(C)c2c1cc(Cc1ccc(F)cc1)c1nc(CF)cn21]

1-(4-(4-fluorobenzyl)-2-(hydroxymethyl)-8,8-dimethyl-7,8-dihydro-6H-imidazo[1,2-a]pyrrolo[2,3-e]pyridin-6-yl)ethan-1-one (0.03 g, 0.082 mmol, 1.0 eq) was dissolved in DCM (5 mL) under argon to give a yellow solution. The reaction mixture was cooled to −78° C. with a dry ice/acetone bath. DAST (0.039 g, 0.245 mmol, 3 eq) was added to the reaction mixture in one portion. The reaction mixture was stirred at −78° C. for 3 h. H₂O (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 4% to give the product (0.015 g, 49.7%) as a yellow oil. MS (ESI) m/z 370.5 [M+H]⁺.

Step 3: 4-(4-fluorobenzyl)-2-(fluoromethyl)-8,8-dimethyl-7,8-dihydro-6H-imidazo[1,2-a]pyrrolo[2,3-e]pyridine

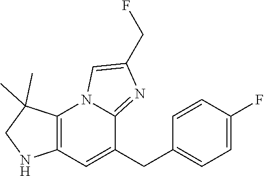 [SMILES: CC1(C)CNc2cc(Cc3ccc(F)cc3)c3nc(CF)cn3c21]

1-(4-(4-fluorobenzyl)-2-(fluoromethyl)-8,8-dimethyl-7,8-dihydro-6H-imidazo[1,2-a]pyrrolo[2,3-e]pyridin-6-yl)ethan-1-one (0.015 g, 0.041 mmol, 1.0 eq) and HCl (6 N, 2 mL) were dissolved in EtOH (4 mL) under argon to give a yellow solution. The reaction mixture was stirred at 82° C. for 2 h. Saturated NaHCO₃ (30 mL) was added to the reaction mixture followed by extraction with ethyl acetate (30 mL×2). The combined organic layers were dried over Na₂SO₄, filtered and concentrated to give the product (0.013 g, 98%) as a yellow oil. MS (ESI) m/z 328.6 [M+H]⁺.

Step 4: 2-chloro-1-(4-(4-fluorobenzyl)-2-(fluoromethyl)-8,8-dimethyl-7,8-dihydro-6H-imidazo[1,2-a]pyrrolo[2,3-e]pyridin-6-yl)ethan-1-one

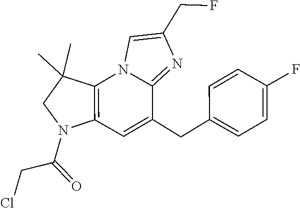 [SMILES: CC1(C)CN(C(=O)CCl)c2cc(Cc3ccc(F)cc3)c3nc(CF)cn3c21]

4-(4-fluorobenzyl)-2-(fluoromethyl)-8,8-dimethyl-7,8-dihydro-6H-imidazo[1,2-a]pyrrolo[2,3-e]pyridine (0.013 g, 0.040 mmol, 1.0 eq), 2-chloroacetyl chloride (6.73 mg, 0.060 mmol, 1.5 eq), and DIEA (10.26 mg, 0.079 mmol, 2.0 eq) were dissolved in DCM (10 mL) to give a yellow solution. The reaction mixture was stirred at RT for 1 h. H₂O (20 mL) was added to the reaction mixture followed by extraction with dichloromethane (30 mL×2). The combined organic layers were dried over Na₂SO₄, filtered and concentrated. The crude product was added to a silica gel column and was eluted with methanol/dichloromethane from 0% to 4% to give the product (0.012 g, 74.8%) as a yellow oil. MS (ESI) m/z 404.7 [M+H]⁺.

Step 5: tert-butyl (2R,5S)-4-(2-(4-(4-fluorobenzyl)-2-(fluoromethyl)-8,8-dimethyl-7,8-dihydro-6H-imidazo[1,2-a]pyrrolo[2,3-e]pyridin-6-yl)-2-oxoethyl)-2-methyl-5-(((R)-3-methylmorpholino)methyl)piperazine-1-carboxylate

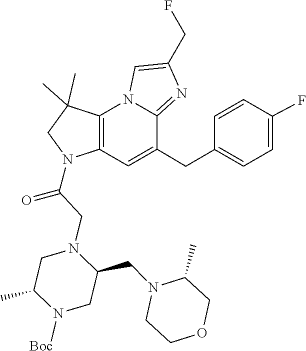 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(CF)cn21]

2-chloro-1-(4-(4-fluorobenzyl)-2-(fluoromethyl)-8,8-dimethyl-7,8-dihydro-6H-imidazo[1,2-a]pyrrolo[2,3-e]pyridin-6-yl)ethan-1-one (0.024 g, 0.059 mmol, 1.0 eq), tert-butyl (2R,5S)-2-methyl-5-(((R)-3-methylmorpholino)methyl)piperazine-1-carboxylate (0.022 g, 0.071 mmol, 1.2 eq), KI (0.015 g, 0.089 mmol, 1.5 eq), and K₂CO₃ (0.025 g, 0.178 mmol, 3.0 eq) were dissolved in Acetonitrile (10 mL) under argon to give a yellow suspension. The reaction mixture was stirred at rt for 5 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.008 g, 19.77%) as a yellow oil. MS (ESI) m/z 681.8 [M+H]⁺.

Step 6: 1-(4-(4-fluorobenzyl)-2-(fluoromethyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl) piperazin-1-yl)ethan-1-one (compound 121)

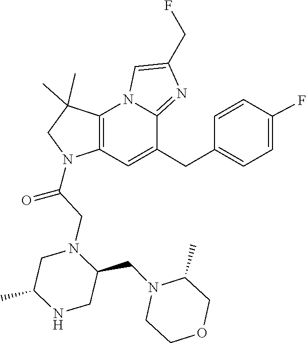 [SMILES: C[C@@H]1CN(CC(=O)N2CC(C)(C)c3c2cc(Cc2ccc(F)cc2)c2nc(CF)cn32)[C@@H](CN2CCOC[C@H]2C)CN1]

Tert-butyl (2R,5S)-4-(2-(4-(4-fluorobenzyl)-2-(fluoromethyl)-8,8-dimethyl-7,8-dihydro-6H-imidazo[1,2-a]pyrrolo[2,3-e]pyridin-6-yl)-2-oxoethyl)-2-methyl-5-(((R)-3-methylmorpholino)methyl)piperazine-1-carboxylate (0.008 g, 0.012 mmol, 1.0 eq) was dissolved in TFA (1 mL) and DCM (3.00 mL) under argon to give a yellow solution. The reaction mixture was stirred at rt for 2 h. Saturated NaHCO₃ (30 mL) was added to the reaction mixture followed by extraction with ethyl acetate (30 mL×2). The combined organic layers were dried over Na₂SO₄, filtered and concentrated. The crude product was purified by pre-HPLC to give the product (1.0 mg, 14.6%) as a white solid. MS (ESI) m/z 581.6 [M+H]⁺.

Example 72: 1-(2-(difluoromethyl)-4-(4-fluorobenzyl)-8,8-dimethyl-7,8-dihydro-6H-imidazo[1,2-a]pyrrolo[2,3-e]pyridin-6-yl)-2-((2R,5R)-5-methyl-2-(((R)-3-methylmorpholino)methyl)piperazin-1-yl)ethan-1-one (compound 122)

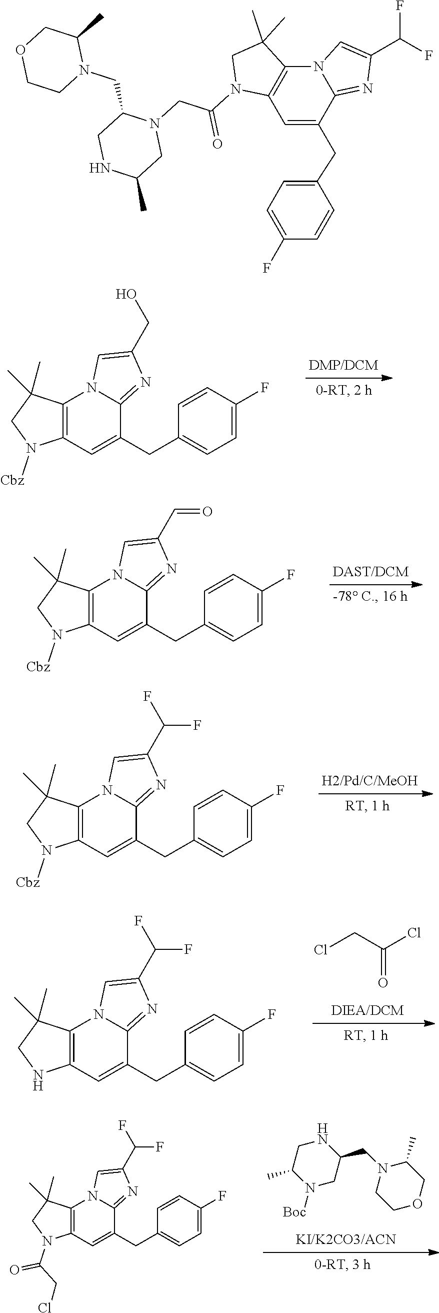

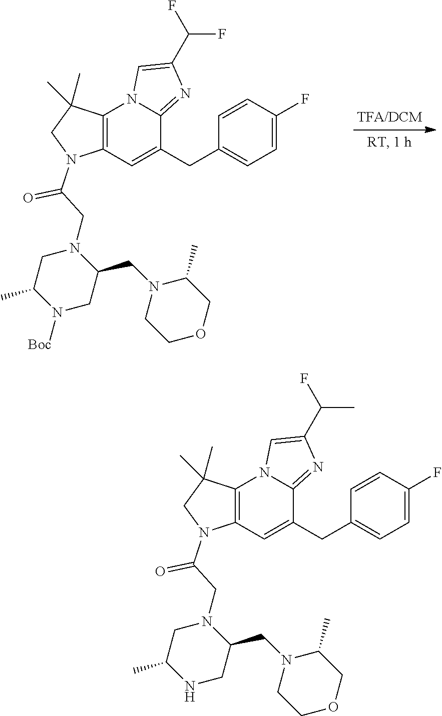 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C(F)F)cn21 ; CC(F)c1cn2c3c(cc(Cc4ccc(F)cc4)c2n1)N(C(=O)CN1C[C@@H](C)NC[C@@H]1CN1CCOC[C@H]1C)CC3(C)C]

Step 1: benzyl 4-(4-fluorobenzyl)-2-formyl-8,8-dimethyl-7,8-dihydro-6H-imidazo[1,2-a]pyrrolo[2,3-e]pyridine-6-carboxylate

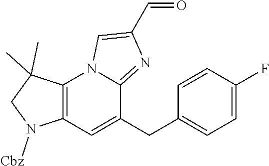 [SMILES: CC1(C)CN(C(=O)OCc2ccccc2)c2cc(Cc3ccc(F)cc3)c3nc(C=O)cn3c21]

Benzyl 4-(4-fluorobenzyl)-2-(hydroxymethyl)-8,8-dimethyl-7,8-dihydro-6H-imidazo[1,2-a]pyrrolo[2,3-e]pyridine-6-carboxylate (0.05 g, 0.109 mmol, 1.0 eq) and DMP (0.069 g, 0.163 mmol, 1.5 eq) were dissolved in DCM (10 mL) under argon to give a yellow solution. The reaction mixture was stirred at rt for 3 h. Saturated NaHCO₃ (20 mL) was added to the reaction mixture followed by extraction with dichloromethane (30 mL×2). The combined organic layers were dried over Na₂SO₄, filtered and concentrated. The crude product was added to a silica gel column and was eluted with ethyl acetate/hexane from 0% to 50% to give the product (0.05 g, 100%) as a yellow oil. MS (ESI) m/z 458.6 [M+H]⁺.

Step 2: benzyl 2-(difluoromethyl)-4-(4-fluorobenzyl)-8,8-dimethyl-7,8-dihydro-6H-imidazo[1,2-a]pyrrolo[2,3-e]pyridine-6-carboxylate

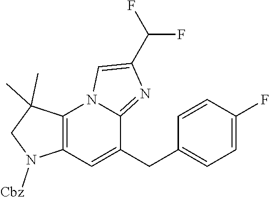 [SMILES: CC1(C)CN(C(=O)OCc2ccccc2)c2cc(Cc3ccc(F)cc3)c3nc(C(F)F)cn3c21]

Benzyl 4-(4-fluorobenzyl)-2-formyl-8,8-dimethyl-7,8-dihydro-6H-imidazo[1,2-a]pyrrolo[2,3-e]pyridine-6-carboxylate (0.05 g, 0.109 mmol, 1.0 eq) was dissolved in DCM (10 mL) under argon to give a yellow solution. The reaction mixture was cooled to −78° C. with a dry ice/acetone bath. DAST (0.053 g, 0.328 mmol, 3 eq) was added to the reaction mixture in one portion. The reaction mixture was stirred at 0° C. for 16 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 40% to give the product (0.044 g, 84%) as a yellow oil. MS (ESI) m/z 480.7 [M+H]⁺.

Step 3: 2-(difluoromethyl)-4-(4-fluorobenzyl)-8,8-dimethyl-7,8-dihydro-6H-imidazo[1,2-a]pyrrolo[2,3-e]pyridine

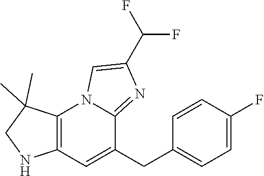 [SMILES: CC1(C)CNc2cc(Cc3ccc(F)cc3)c3nc(C(F)F)cn3c21]

Benzyl 2-(difluoromethyl)-4-(4-fluorobenzyl)-8,8-dimethyl-7,8-dihydro-6H-imidazo[1,2-a]pyrrolo[2,3-e]pyridine-6-carboxylate (0.044 g, 0.092 mmol, 1.0 eq) and Pd/C (0.020 g, 0.184 mmol, 2 eq) were dissolved in MeOH (5 mL) under H₂ to give a black suspension. The reaction mixture was stirred at rt for 1 h. The crude product was added to a silica gel column and was eluted with ethyl acetate/hexane from 0% to 50% to give the product (0.032 g, 100%) as a yellow oil. MS (ESI) m/z 346.4 [M+H]⁺.

Steps 4-6: Following the synthesis of compound 14, compound 122 was obtained as a white solid (2.1 mg). ¹H NMR (400 MHz, DMSO-d⁶) δ (ppm) 8.05-7.94 (m, 2H), 7.36-7.28 (m, 2H), 7.12-7.03 (m, 2H), 4.04-3.89 (m, 3H), 3.83-3.75 (m, 1H), 3.73-3.60 (m, 2H), 3.50 (d, J=10.2 Hz, 2H), 3.10-3.00 (m, 2H), 2.96-2.89 (m, 1H), 2.86-2.75 (m, 3H), 2.72-2.60 (m, 3H), 2.37-2.28 (m, 2H), 2.21-2.10 (m, 1H), 1.98-1.87 (m, 1H), 1.77 (d, J=11.6 Hz, 1H), 1.58 (d, J=12.1 Hz, 6H), 0.91 (dd, J=9.0, 6.3 Hz, 6H). MS (ESI) m/z 599.8 [M+H]⁺.

Example 73: 1-(4-(2-chloro-4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 123)

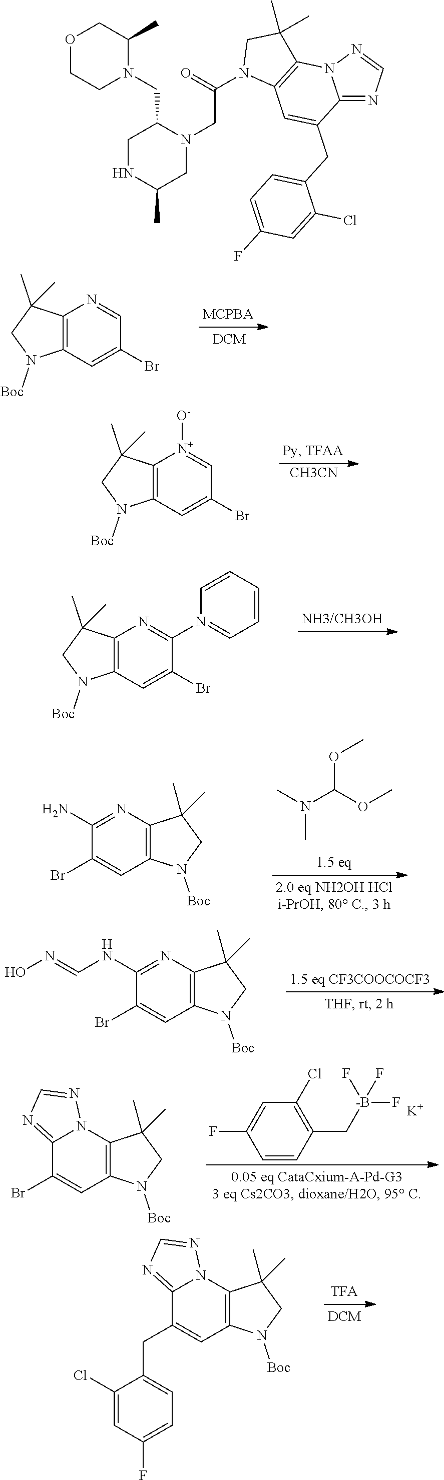

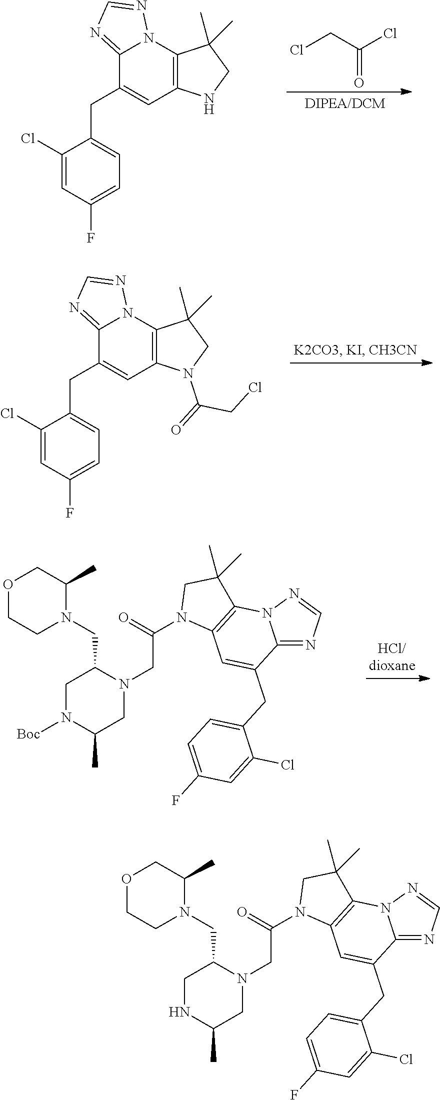

Step 1: tert-butyl 6-bromo-3,3-dimethyl-4-(1l-oxidanyl)-2,3-dihydro-1H-414-pyrrolo[3,2-b]pyridine-1-carboxylate

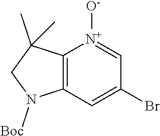 [SMILES: CC(C)(C)OC(=O)N1CC(C)(C)c2c1cc(Br)c[n+]2[O-]]

To a solution of tert-butyl 6-bromo-3,3-dimethyl-2,3-dihydro-1H-pyrrolo[3,2-b]pyridine-1-carboxylate (5 g, 15.28 mmol, 1.0 eq) in CH₂Cl₂ (15 mL) were added 1-chloro-3-hydroperoxybenzene (2.430 g, 16.81 mmol, 1.1 eq) at 0° C. and stirred at 25° C. for 16 h under N₂, To the reaction mixture were added sat NaHCO₃aq and ethyl acetate. The combined organic layer was washed with brine (100 mL), dried over Na₂SO₄, filtered and concentrated to give the title compound (5.24 g, 100%) as a yellow solid. MS (ESI) m/z 344.6[M+H]⁺.

Step 2: 1-(6-bromo-1-(tert-butoxycarbonyl)-3,3-dimethyl-2,3-dihydro-1H-pyrrolo[3,2-b]pyrid in-5-yl)pyridin-1-ium

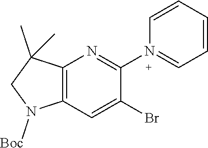

To a solution of 6-bromo-1-(tert-butoxycarbonyl)-3,3-dimethyl-2,3-dihydro-1H-pyrrolo[3,2-b]pyridine 4-oxide (5.26 g, 15.33 mmol, 1.0 eq) in MeCN (80 mL) was added pyridine (6.06 g, 77 mmol, 5.0 eq) at 0° C., then 2,2,2-trifluoroacetic anhydride (9.66 g, 46.0 mmol, 3.0 eq) was added at 0° C. and stirred at rt for 16 h under N₂, To the mixture was concentrated to give the title compound as a yellow oil, which was used in next step directly. MS (ESI) m/z 405.8[M+H]⁺.

Step 3: tert-butyl 5-amino-6-bromo-3,3-dimethyl-2,3-dihydro-1H-pyrrolo[3,2-b]pyridine-1-carboxylate

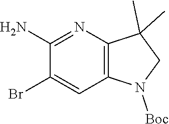 [SMILES: CC(C)(C)OC(=O)N1CC(C)(C)c2nc(N)c(Br)cc21]

To a solution of 1-(6-bromo-1-(tert-butoxycarbonyl)-3,3-dimethyl-2,3-dihydro-1H-pyrrolo[3,2-b]pyrid in-5-yl)pyridin-1-ium (6.21 g, 15.32 mmol, 1.0 eq) in NH₃ in CH₃OH (50 mL) was stirred at 80° C. for 3 h under sealed tube, Then the mixture was concentrated and purified by silica gel flash column chromatography eluting with PE/EA=5/1 to give the title compound (4.7 g, 90%) as a yellow oil. MS (ESI) m/z 343.6[M+H]+.

Step 4: tert-butyl (E)-6-bromo-5-(N′-hydroxyformimidamido)-3,3-dimethyl-2,3-dihydro-1H-pyrrolo[3,2-b]pyridine-1-carboxylate

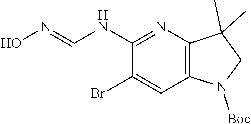 [SMILES: CC(C)(C)OC(=O)N1CC(C)(C)c2nc(N/C=N/O)c(Br)cc21]

To a solution of tert-butyl 5-amino-6-bromo-3,3-dimethyl-2,3-dihydro-1H-pyrrolo[3,2-b]pyridine-1-carboxylate (100 mg, 0.292 mmol, 1.0 eq) in 2-propanol (15 mL) was added 1,1-dimethoxy-N,N-dimethylmethanamine (52.2 mg, 0.438 mmol, 1.5 eq) at 25° C. The mixture was heated to 80° C. for 2 h under N₂, then hydroxylamine hydrochloride (40.6 mg, 0.584 mmol, 2.0 eq) was added at rt. The mixture was heated to 50° C. for 1 h under N₂. After concentration, the residue was used in next step directly.

Step 5: tert-butyl 4-bromo-8,8-dimethyl-7,8-dihydro-6H-pyrrolo[2,3-e][1,2,4]triazolo[1,5-a]pyridine-6-carboxylate

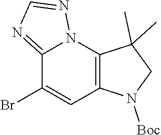 [SMILES: CC(C)(C)OC(=O)N1CC(C)(C)c2c1cc(Br)c1ncnn21]

To a solution of tert-butyl (E)-6-bromo-5-(N′-hydroxyformimidamido)-3,3-dimethyl-2,3-dihydro-1H-pyrrolo[3,2-b]pyridine-1-carboxylate (113 mg, 0.293 mmol, 1.0 eq) in THF (15 mL) was added 2,2,2-trifluoroacetic anhydride (92 mg, 0.440 mmol, 1.5 eq) at 25° C. The mixture was stirred at 25° C. for 2 h under N₂, then the solution was concentrated. The crude product was added to a silica gel column and eluted with methanol/dichloromethane=1:20 to give the title compound (100 mg, 93%) as a white solid. MS (ESI) m/z 368.6 [M+H]⁺.

Step 6: tert-butyl 4-(2-chloro-4-fluorobenzyl)-8,8-dimethyl-7,8-dihydro-6H-pyrrolo[2,3-e][1,2,4]triazolo[1,5-a]pyridine-6-carboxylate

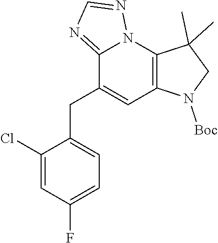 [SMILES: CC(C)(C)OC(=O)N1CC(C)(C)c2c1cc(Cc1ccc(F)cc1Cl)c1ncnn21]

To a solution of tert-butyl 4-bromo-8,8-dimethyl-7,8-dihydro-6H-pyrrolo[2,3-e][1,2,4]triazolo[1,5-a]pyridine-6-carboxylate (100 mg, 0.272 mmol, 1.0 eq) in 1,4-dioxane (10 mL)/water (1.0 mL) were added potassium (2-chloro-4-fluorobenzyl)trifluoroborate (102 mg, 0.408 mmol, 1.5 eq), CataCxium-A-Pd-G3 (9.92 mg, 0.014 mmol, 0.05 eq), and Cs₂CO₃ (266 mg, 0.817 mmol, 3.0 eq) at 25° C. The mixture was stirred at 95° C. for 8.0 h under N₂. H₂O was added to the reaction mixture followed by extraction with ethyl acetate. The combined organic layers were dried over Na₂SO₄, filtered and concentrated. The crude product was added to a silica gel column and eluted with ethyl acetate/hexane (ethyl acetate:hexane=1:1) to give the title compound (70 mg, 59.7%) as a yellow solid.

Step 7: 4-(2-chloro-4-fluorobenzyl)-8,8-dimethyl-7,8-dihydro-6H-pyrrolo[2,3-e][1,2,4]triazolo[1,5-a]pyridine

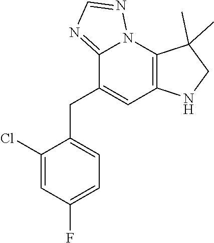 [SMILES: CC1(C)CNc2cc(Cc3ccc(F)cc3Cl)c3ncnn3c21]

To a solution of tert-butyl 4-(2-chloro-4-fluorobenzyl)-8,8-dimethyl-7,8-dihydro-6H-pyrrolo[2,3-e][1,2,4]triazolo[1,5-a]pyridine-6-carboxylate (70 mg, 0.162 mmol, 1.0 eq) in CH₂Cl₂ (10 mL) was added TFA (5 mL) at 25° C. The mixture was stirred at 25° C. for 2 h. After concentration, saturated NaHCO₃(aq) and ethyl acetate were added. The organic layer was washed with brine (100 mL), dried over Na₂SO₄, filtered and concentrated to give the title compound (48 mg, 89%) as a yellow solid.

Step 8: 2-chloro-1-(4-(2-chloro-4-fluorobenzyl)-8,8-dimethyl-7,8-dihydro-6H-pyrrolo[2,3-e][1,2,4]triazolo[1,5-a]pyridin-6-yl)ethan-1-one

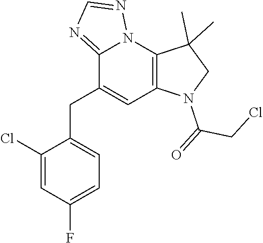 [SMILES: CC1(C)CN(C(=O)CCl)c2cc(Cc3ccc(F)cc3Cl)c3ncnn3c21]

To a solution of 4-(2-chloro-4-fluorobenzyl)-8,8-dimethyl-7,8-dihydro-6H-pyrrolo[2,3-e][1,2,4]triazolo[1,5-a]pyridine (48 mg, 0.145 mmol, 1.0 eq) in CH₂Cl₂ (15 mL) was added N-ethyl-N-isopropylpropan-2-amine (56.3 mg, 0.435 mmol, 3.0 eq) at 0° C. 2-chloroacetyl chloride (19.67 mg, 0.174 mmol, 1.2 eq) was added at 0° C. and the mixture was stirred at rt for 3 h under N₂. Water and DCM were added. The combined organic layer was washed with brine (100 mL), dried over Na₂SO₄, filtered and concentrated. The crude product was added to a silica gel column and eluted with DCM/CH₃OH (50:1) to give the title compound (20 mg, 33.8%) as a white solid. MS (ESI) m/z 407.2 [M+H]⁺.

Step 9: tert-butyl (2R,5S)-4-(2-(4-(2-chloro-4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

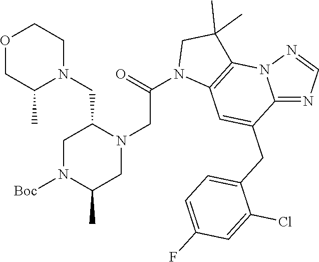 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1Cl)c1ncnn21]

To a solution of 2-chloro-1-(4-(2-chloro-4-fluorobenzyl)-8,8-dimethyl-7,8-dihydro-6H-pyrrolo[2,3-e][1,2,4]triazolo[1,5-a]pyridin-6-yl)ethan-1-one (20 mg, 0.049 mmol, 1.0 eq) in acetonitrile (15 mL) was added tert-butyl (2R,5S)-2-methyl-5-(((R)-3-methylmorpholino) methyl) piperazine-1-carboxylate (15.39 mg, 0.049 mmol, 1.0 eq) at 25° C. Potassium iodide (12.23 mg, 0.074 mmol, 1.5 eq), and K₂CO₃ (20.36 mg, 0.147 mmol, 3.0 eq) were added at 25° C. and the mixture was stirred at rt for 3 h under N₂. Water and ethyl acetate were added. The combined organic layer was washed with brine (100 mL), dried over Na₂SO₄, filtered and concentrated. The residue was purified by silica gel flash column chromatography and eluted with DCM/CH₃OH (50/1) to give the title compound (17 mg, 50.6%) as a yellow solid. MS (ESI) m/z 684.5 [M+H]⁺.

Step 10: 1-(4-(2-chloro-4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino) methyl)piperazin-1-yl)ethan-1-one (Compound 123)

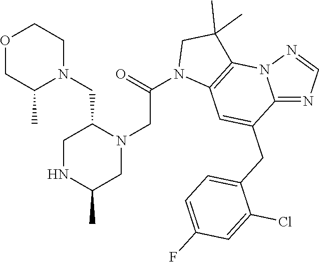 [SMILES: C[C@@H]1CN(CC(=O)N2CC(C)(C)c3c2cc(Cc2ccc(F)cc2Cl)c2ncnn32)[C@@H](CN2CCOC[C@H]2C)CN1]

To a solution of tert-butyl (2R,5S)-4-(2-(4-(2-chloro-4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (17 mg, 0.025 mmol, 1.0 eq) in HCl/dioxane (3 mL) was stirred at 25° C. for 30 min. The solvent was evaporated and 7.0 N NH₃ in CH₃OH solution (5 mL) was added. After concentration, the residue was purified by pre-HPLC to give the title compound (2.5 mg, 17.23%) as a white solid. MS (ESI) m/z 584.5 [M+H]⁺. ¹H NMR (400 MHz, DMSO-d⁶) δ (ppm) 8.52 (s, 1H), 8.20 (s, 1H), 7.57-7.44 (m, 2H), 7.28-7.15 (m, 1H), 4.48-4.31 (m, 2H), 4.12 (d, J=10.6 Hz, 1H), 3.92 (d, J=10.5 Hz, 1H), 3.80-3.64 (m, 2H), 3.47-3.43 (m, 2H), 3.16-2.99 (m, 2H), 2.91-2.60 (m, 6H), 2.59-2.53 (m, 1H), 2.35-2.23 (m, 1H), 2.19-2.08 (m, 1H), 2.04-1.84 (m, 1H), 1.76 (d, J=12.4 Hz, 1H), 1.68-1.53 (m, 6H), 0.96-0.84 (m, 6H).

Example 74: 1-(4-(4-fluorobenzyl)-1,3,8,8-tetramethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (Compound 124)

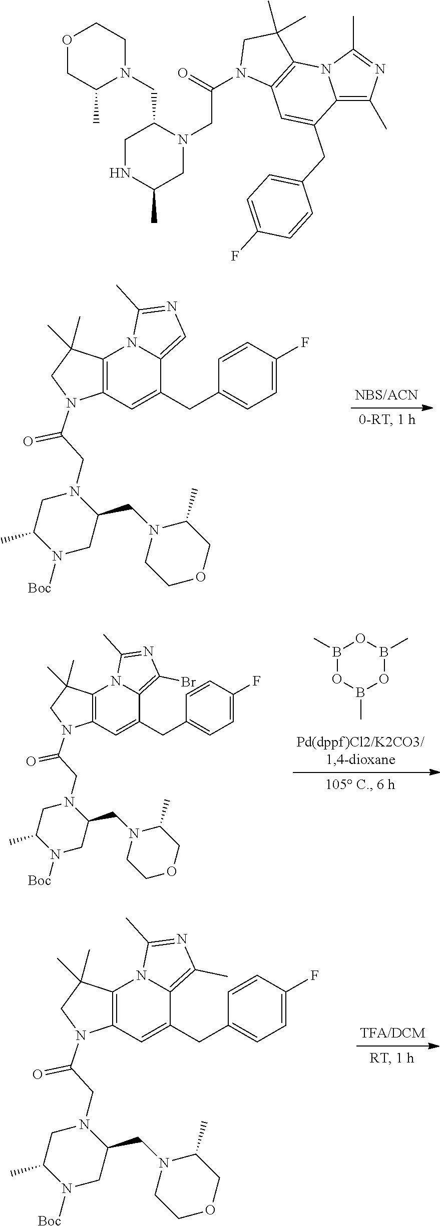

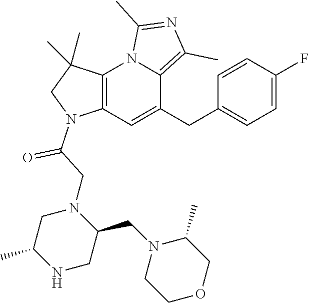 [SMILES: Cc1nc(C)n2c3c(cc(Cc4ccc(F)cc4)c12)N(C(=O)CN1C[C@@H](C)NC[C@@H]1CN1CCOC[C@H]1C)CC3(C)C]

Step 1: tert-butyl (2R,5S)-4-(2-(3-bromo-4-(4-fluorobenzyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

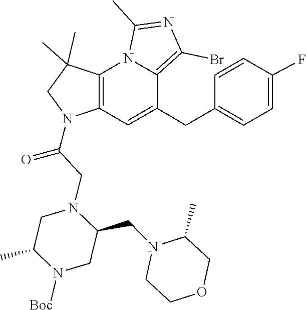 [SMILES: Cc1nc(Br)c2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C]

Tert-butyl (2R,5S)-4-(2-(4-(4-fluorobenzyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (0.038 g, 0.057 mmol, 1.0 eq) and NBS (10.20 mg, 0.057 mmol, 1.0 eq) were dissolved in Acetonitrile (5 mL) under argon to give a yellow solution. The reaction mixture was stirred at rt for 1 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.02 g, 47.0%) as a yellow oil. MS (ESI) m/z 741.5 [M+H]⁺.

Step 2: tert-butyl (2R,5S)-4-(2-(4-(4-fluorobenzyl)-1,3,8,8-tetramethyl-7,8-dihydro-6H-imidazo[1,5-a]pyrrolo[2,3-e]pyridin-6-yl)-2-oxoethyl)-2-methyl-5-(((R)-3-methylmorpholino)methyl) piperazine-1-carboxylate

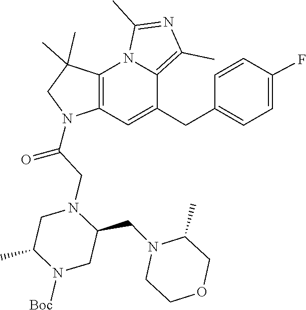 [SMILES: Cc1nc(C)n2c3c(cc(Cc4ccc(F)cc4)c12)N(C(=O)CN1C[C@@H](C)N(C(=O)OC(C)(C)C)C[C@@H]1CN1CCOC[C@H]1C)CC3(C)C]

Tert-butyl (2R,5S)-4-(2-(3-bromo-4-(4-fluorobenzyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (0.02 g, 0.027 mmol, 1.0 eq), 2,4,6-trimethyl-1,3,5,2,4,6-trioxatriborinane (0.017 g, 0.135 mmol, 5.0 eq), K₂CO₃ (0.011 g, 0.081 mmol, 3.0 eq) and PdCl₂(dppf) (3.95 mg, 5.39 μmol, 0.2 eq) were dissolved in water (0.500 mL) and 1,4-dioxane (5 mL) under argon to give a brown suspension. The reaction mixture was stirred at 100° C. for 6 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.006 g, 32.9%) as a yellow oil. MS (ESI) m/z 677.8 [M+H]⁺.

Step 3: 1-(4-(4-fluorobenzyl)-1,3,8,8-tetramethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one

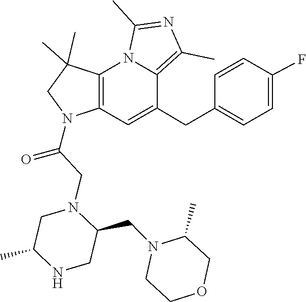 [SMILES: Cc1nc(C)n2c3c(cc(Cc4ccc(F)cc4)c12)N(C(=O)CN1C[C@@H](C)NC[C@@H]1CN1CCOC[C@H]1C)CC3(C)C]

Tert-butyl (2R,5S)-4-(2-(4-(4-fluorobenzyl)-1,3,8,8-tetramethyl-7,8-dihydro-6H-imidazo[1,5-a]pyrrolo[2,3-e]pyridin-6-yl)-2-oxoethyl)-2-methyl-5-(((R)-3-methylmorpholino)methyl) piperazine-1-carboxylate (0.01 g, 0.015 mmol, 1.0 eq) was dissolved in TFA (1 mL) and DCM (3.00 mL) under argon to give a yellow solution. The reaction mixture was stirred at rt for 1 h. Saturated NaHCO₃ (30 mL) was added to the reaction mixture followed by extraction with ethyl acetate (30 mL×2). The combined organic layers were dried over Na₂SO₄, filtered and concentrated. The crude product was purified by column chromatography to give the product (1.0 mg, 11.7%) as a white solid. MS (ESI) m/z 577.6 [M+H]⁺.

Example 75: tert-butyl (2R,5S)-4-(2-(4-(4-fluorobenzyl)-3-(hydroxymethyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (compound 125)

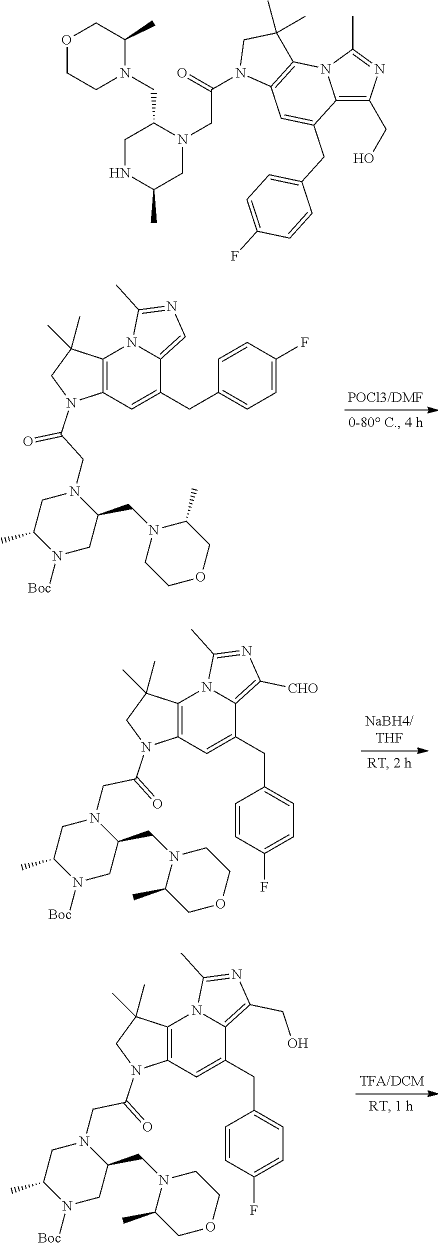

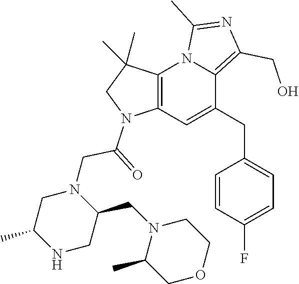 [SMILES: Cc1nc(CO)c2c(Cc3ccc(F)cc3)cc3c(n12)C(C)(C)CN3C(=O)CN1C[C@@H](C)NC[C@@H]1CN1CCOC[C@H]1C]

Step 1: tert-butyl (2R,5S)-4-(2-(4-(4-fluorobenzyl)-3-formyl-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

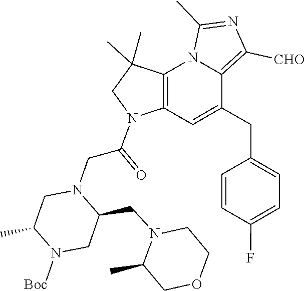 [SMILES: Cc1nc(C=O)c2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C]

Tert-butyl (2R,5S)-4-(2-(4-(4-fluorobenzyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (0.045 g, 0.068 mmol, 1.0 eq) and POCl₃ (0.021 g, 0.136 mmol, 2.0 eq) were dissolved in DMF (3 mL) under argon to give a yellow solution. The reaction mixture was stirred at 80° C. for 3 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.012 g, 25.6%) as a yellow oil. MS (ESI) m/z 691.7 [M+H]⁺.

Step 2: tert-butyl (2R,5S)-4-(2-(4-(4-fluorobenzyl)-3-(hydroxymethyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate

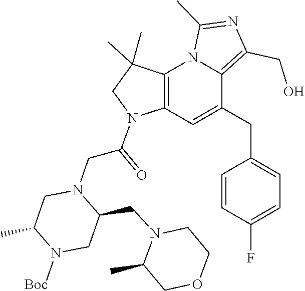 [SMILES: Cc1nc(CO)c2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C]

Tert-butyl (2R,5S)-4-(2-(4-(4-fluorobenzyl)-3-formyl-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (0.012 g, 0.017 mmol, 1.0 eq) and NaBH₄ (1.971 mg, 0.052 mmol, 3.0 eq) were dissolved in THF (5 mL) under argon to give a yellow solution. The reaction mixture was stirred at rt for 3 h. Saturated NH₄Cl (10 mL) was added to the reaction mixture followed by extraction with ethyl acetate (30 mL×2). The combined organic layers were dried over Na₂SO₄, filtered and concentrated to give the product (0.012 g, 100%). MS (ESI) m/z 693.8 [M+H]⁺.

Step 3: 1-(4-(4-fluorobenzyl)-3-(hydroxymethyl)-1,8,8-trimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (compound 125)

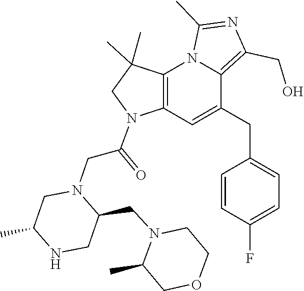 [SMILES: Cc1nc(CO)c2c(Cc3ccc(F)cc3)cc3c(n12)C(C)(C)CN3C(=O)CN1C[C@@H](C)NC[C@@H]1CN1CCOC[C@H]1C]

Tert-butyl (2R,5S)-4-(2-(4-(4-fluorobenzyl)-3-(hydroxymethyl)-1,8,8-trimethyl-7,8-dihydro-6H-imidazo[1,5-a]pyrrolo[2,3-e]pyridin-6-yl)-2-oxoethyl)-2-methyl-5-(((R)-3-methylmorpholino)methyl)piperazine-1-carboxylate (0.012 g, 0.017 mmol, 1.0 eq) was dissolved in TFA (1 mL) and DCM (3.00 mL) under argon to give a yellow solution. The reaction mixture was stirred at rt for 1 h. Saturated NaHCO₃ (30 mL) was added to the reaction mixture followed by extraction with ethyl acetate (30 mL×2). The combined organic layers were dried over Na₂SO₄, filtered and concentrated. The crude product was purified by column chromatography to give the product (0.3 mg, 2.9%) as a white solid. MS (ESI) m/z 593.6 [M+H]⁺.

Example 76: 1-(4-(4-fluorobenzyl)-2-methoxy-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 126)

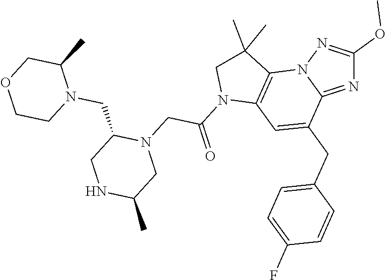 [SMILES: COc1nc2c(Cc3ccc(F)cc3)cc3c(n2n1)C(C)(C)CN3C(=O)CN1C[C@@H](C)NC[C@@H]1CN1CCOC[C@H]1C]

Step 1: benzyl 4-(4-fluorobenzyl)-2-methoxy-8,8-dimethyl-7,8-dihydro-6H-pyrrolo[2,3-e][1,2,4]triazolo[1,5-a]pyridine-6-carboxylate

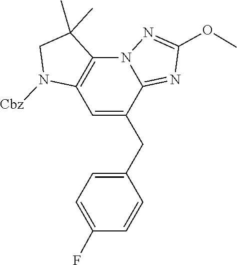 [SMILES: COc1nc2c(Cc3ccc(F)cc3)cc3c(n2n1)C(C)(C)CN3C(=O)OCc1ccccc1]

To a solution of benzyl 4-(4-fluorobenzyl)-2-hydroxy-8,8-dimethyl-7,8-dihydro-6H-pyrrolo[2,3-e][1,2,4]triazolo[1,5-a]pyridine-6-carboxylate (0.1 g, 0.22 mmol) in MeOH (2.0 ml) was added K₂CO₃ (0.062 g, 0.45 mmol) and iodomethane (0.064 g, 0.45 mmol). The mixture was stirred at r.t. for 1 h. The mixture was diluted with ethyl acetate and then washed with H₂O, and saturated NaCl. The organic layer was dried over Na₂SO₄, filtered and concentrated. The crude product was added to a silica gel column and was eluted with ethyl acetate/hexane (1:3) to give the title compound (80 mg, 78%) as a yellow oil. MS: 461.2 (M+H⁺).

Following steps: Compound 126 was obtained as a white solid in a manner similar to that described in compound 127. ¹H NMR (400 MHz, CD₃OD) δ (ppm) 8.27 (s, 1H), 7.36-7.24 (m, 2H), 7.08-6.93 (m, 2H), 4.23-4.16 (m, 2H), 4.09-4.02 (m, 3H), 3.66-3.52 (m, 4H), 3.27 (s, 3H), 3.08-2.82 (m, 7H), 2.55-2.42 (m, 2H), 2.30-2.17 (m, 1H), 2.07-1.99 (m, 1H), 1.94-1.80 (m, 1H), 1.70-1.60 (m, 6H), 1.06-0.91 (m, 6H). MS: 580.3 (M+H⁺).

Example 77: 4-(4-fluorobenzyl)-1,8,8-trimethyl-6-(2-((2R,5R)-5-methyl-2-(((R)-3-methylmorpholino)methyl)piperazin-1-yl)acetyl)-7,8-dihydro-1H-pyrrolo[2,3-e][1,2,4]triazolo[1,5-a]pyridin-2(6H)-one (compound 127)

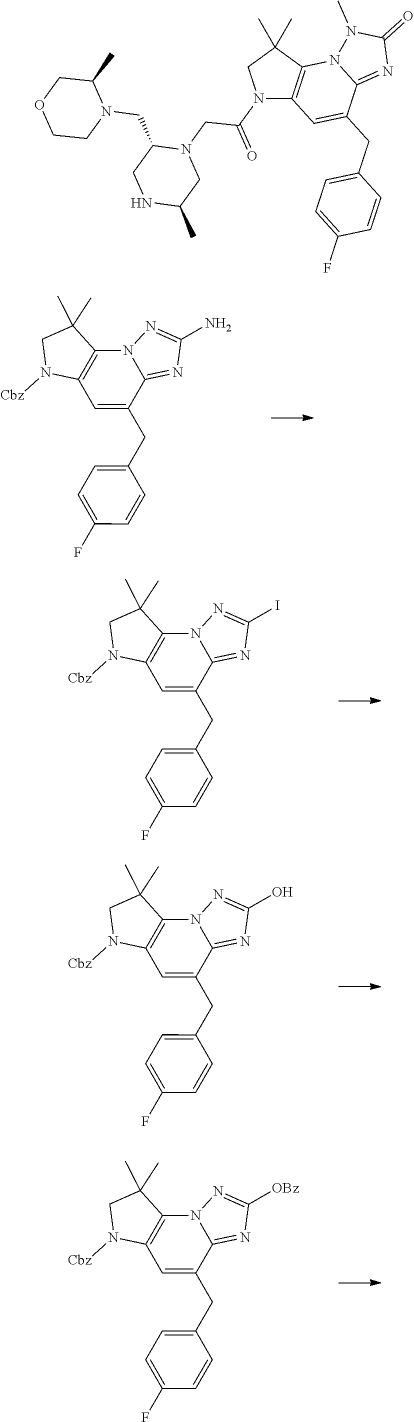

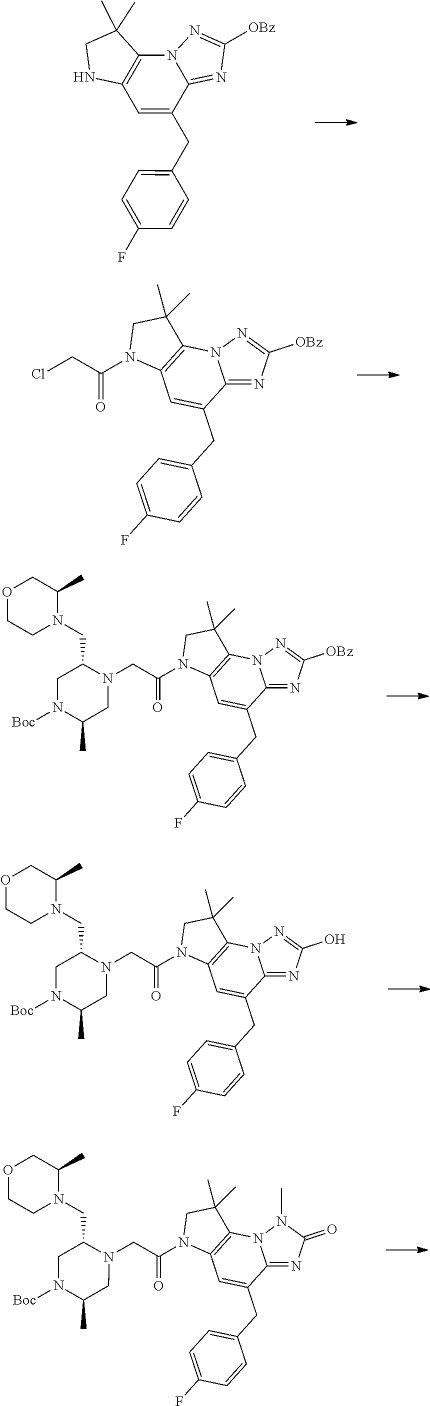

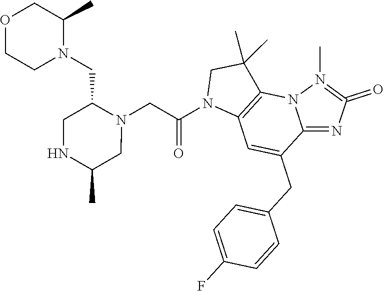

Step 1: benzyl 4-(4-fluorobenzyl)-2-iodo-8,8-dimethyl-7,8-dihydro-6H-pyrrolo[2,3-e][1,2,4]triazolo[1,5-a]pyridine-6-carboxylate

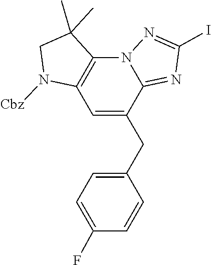 [SMILES: CC1(C)CN(C(=O)OCc2ccccc2)c2cc(Cc3ccc(F)cc3)c3nc(I)nn3c21]

To a solution of sodium nitrite (155 mg, 2.25 mmol) and KI (373 mg, 2.25 mmol) in water (3.0 ml) was added a solution of 4-methylbenzenesulfonic acid (464 mg, 2.69 mmol) and benzyl 2-amino-4-(4-fluorobenzyl)-8,8-dimethyl-7,8-dihydro-6H-pyrrolo[2,3-e][1,2,4]triazolo[1,5-a]pyridine-6-carboxylate (200 mg, 0.45 mmol) in acetonitrile (3.0 ml) at 0° C. The mixture was stirred at r.t. for 2 h. The mixture was quenched by Sat·NaHCO₃·aq and The reaction mixture was diluted with ethyl acetate and then washed with H₂O, and saturated NaCl. The organic layer was dried over Na₂SO₄, filtered and concentrated. The crude product was added to a silica gel column and was eluted with ethyl acetate/hexane (1:5) to give the title compound (0.2 g, 80%) as a yellow oil. MS: 557.1 (M+H⁺).

Step 2: benzyl 4-(4-fluorobenzyl)-2-hydroxy-8,8-dimethyl-7,8-dihydro-6H-pyrrolo[2,3-e][1,2,4]triazolo[1,5-a]pyridine-6-carboxylate

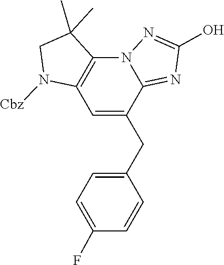 [SMILES: CC1(C)CN(C(=O)OCc2ccccc2)c2cc(Cc3ccc(F)cc3)c3nc(O)nn3c21]

To a solution of benzyl 4-(4-fluorobenzyl)-2-iodo-8,8-dimethyl-7,8-dihydro-6H-pyrrolo[2,3-e][1,2,4]triazolo[1,5-a]pyridine-6-carboxylate (0.2 g, 0.36 mmol) in 1,4-dioxane (5.0 ml)/water (0.5 ml) was added sodium 2-methylpropan-2-olate (104 mg, 1.08 mmol) and PdCl₂(dppf)-CH₂Cl₂ adduct (29 mg, 0.036 mmol). The mixture was filled with N₂ and the mixture was stirred at 110° C. for 2 h. The mixture was cooled to r.t. and diluted with Sat·NH₄Cl·aq and then washed with H₂O and saturated brine. The organic layer was dried over Na₂SO₄, filtered and concentrated. The crude product was added to a silica gel column and eluted with methanol/dichloromethane (1:10) to give the title compound (0.1 g, 62.3%) as a yellow oil. MS: 447.2 (M+H⁺).

Step 3: benzyl 2-(benzoyloxy)-4-(4-fluorobenzyl)-8,8-dimethyl-7,8-dihydro-6H-pyrrolo[2,3-e][1,2,4]triazolo[1,5-a]pyridine-6-carboxylate

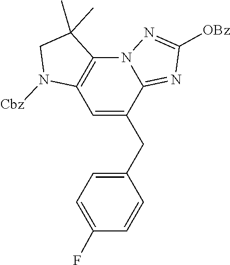

To a solution of benzyl 4-(4-fluorobenzyl)-2-hydroxy-8,8-dimethyl-7,8-dihydro-6H-pyrrolo[2,3-e][1,2,4]triazolo[1,5-a]pyridine-6-carboxylate (0.35 g, 0.79 mmol) in DCM (5.0 ml) was added TEA (0.16 g, 1.58 mmol) and benzoyl chloride (0.16 g, 1.18 mmol) at 0° C. The mixture was stirred at 20° C. for 2 h. The mixture was cooled to room temperature and diluted with sat·NaHCO₃·aq and then washed with H₂O and saturated brine. The organic layer was dried over Na₂SO₄, filtered and concentrated. The crude product was added to a silica gel column and was eluted with ethyl acetate/hexane (1:3) to give the title compound (0.26 g, 60.2%) as a white solid. MS: 551.2 (M+H⁺).

Step 4: 4-(4-fluorobenzyl)-8,8-dimethyl-7,8-dihydro-6H-pyrrolo[2,3-e][1,2,4]triazolo[1,5-a]pyridin-2-yl benzoate

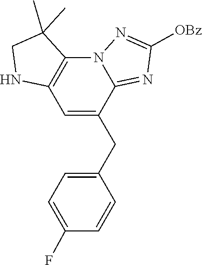

To a solution of benzyl 2-(benzoyloxy)-4-(4-fluorobenzyl)-8,8-dimethyl-7,8-dihydro-6H-pyrrolo[2,3-e][1,2,4]triazolo[1,5-a]pyridine-6-carboxylate (260 mg, 0.47 mmol) in MeOH (10 ml) was added Pd/C (10%, 20 mg). The mixture was filled H₂ three times and stirred at r.t. for 2 h. The mixture was filtrated and concentrated. The crude product was added to a silica gel column and was eluted with methanol/dichloromethane (1:20) to give 4-(4-fluorobenzyl)-8,8-dimethyl-7,8-dihydro-6H-pyrrolo[2,3-e][1,2,4]triazolo[1,5-a]pyridin-2-yl benzoate (180 mg, 92%) as a colorless oil. MS: 417.2 (M+H⁺).

Step 5: 6-(2-chloroacetyl)-4-(4-fluorobenzyl)-8,8-dimethyl-7,8-dihydro-6H-pyrrolo[2,3-e][1,2,4]triazolo[1,5-a]pyridin-2-yl benzoate

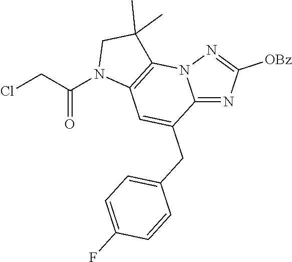

To a solution of 4-(4-fluorobenzyl)-8,8-dimethyl-7,8-dihydro-6H-pyrrolo[2,3-e][1,2,4]triazolo[1,5-a]pyridin-2-yl benzoate (180 mg, 0.44 mmol) in DCM (3.0 ml) was added 2-chloroacetyl chloride (58.6 mg, 0.52 mmol). The mixture was stirred at r.t. for 1 h. The mixture was concentrated to give crude product 195 mg, which was used next step directly. MS: 493.1 (M+H⁺).

Step 6: tert-butyl (2R,5S)-4-(2-(2-(benzoyloxy)-4-(4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

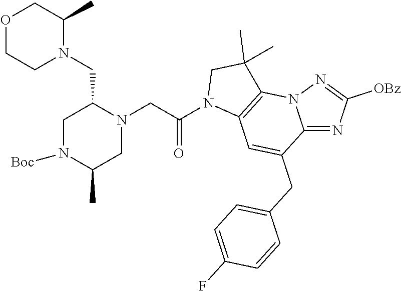

To a solution of 6-(2-chloroacetyl)-4-(4-fluorobenzyl)-8,8-dimethyl-7,8-dihydro-6H-pyrrolo[2,3-e][1,2,4]triazolo[1,5-a]pyridin-2-yl benzoate (195 mg, 0.4 mmol) in acetonitrile (3.0 ml) was added tert-butyl (2R,5S)-2-methyl-5-(((R)-3-methylmorpholino)methyl)piperazine-1-carboxylate (124 mg, 0.4 mmol), potassium iodide (99 mg, 0.6 mmol) and K₂CO₃ (164 mg, 1.2 mmol). The mixture was stirred at 29° C. for 2 h. The mixture was filtrated and concentrated to give crude product 230 mg, which was used next step directly. MS: 770.4 (M+H⁺).

Step 7: tert-butyl (2R,5S)-4-(2-(4-(4-fluorobenzyl)-2-hydroxy-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

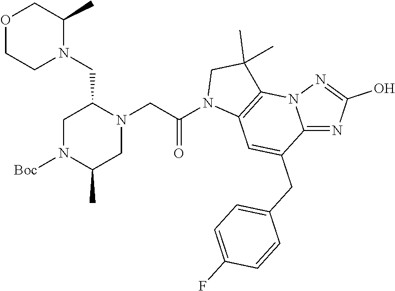 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(O)nn21]

To a solution of tert-butyl (2R,5S)-4-(2-(2-(benzoyloxy)-4-(4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (230 mg, 0.3 mmol) in MeOH was added NH₃. The mixture was stirred at 29° C. for 2 h. The mixture was filtrated and concentrated to give crude product. The crude product was added to a silica gel column and was eluted with methanol/dichloromethane (1:10) to give the title compound (150 mg, 75%) as a colorless oil. MS: 666.4 (M+H⁺).

Step 8: tert-butyl (2R,5S)-4-(2-(4-(4-fluorobenzyl)-1,8,8-trimethyl-2-oxo-1,2,7,8-tetrahydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

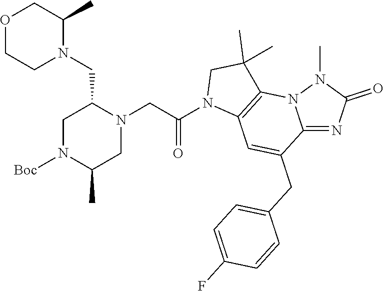 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(=O)n(C)n21]

To a solution of tert-butyl (2R,5S)-4-(2-(4-(4-fluorobenzyl)-2-hydroxy-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (100 mg, 0.15 mmol) in MeOH (2 ml) was added iodomethane (426 mg, 3.0 mmol) and Cs₂CO₃ (245 mg, 0.75 mmol). The mixture was stirred at 80° C. for 12 h. The mixture was concentrated and the crude product was added to a silica gel column and was eluted with DCM/MeOH (10:1) to give the title compound (38 mg, 37.2%) as a white solid. MS: 680.4 (M+H⁺).

Step 9: 4-(4-fluorobenzyl)-1,8,8-trimethyl-6-(2-((2R,5R)-5-methyl-2-(((R)-3-methylmorpholino)methyl)piperazin-1-yl)acetyl)-7,8-dihydro-1H-pyrrolo[2,3-e][1,2,4]triazolo[1,5-a]pyridin-2(6H)-one (compound 127)

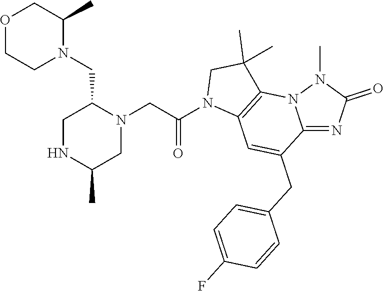 [SMILES: C[C@@H]1CN(CC(=O)N2CC(C)(C)c3c2cc(Cc2ccc(F)cc2)c2nc(=O)n(C)n32)[C@@H](CN2CCOC[C@H]2C)CN1]

To a solution of tert-butyl (2R,5S)-4-(2-(4-(4-fluorobenzyl)-1,8,8-trimethyl-2-oxo-1,2,7,8-tetrahydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (38 mg, 0.06 mmol) in DCM (2.0 ml) was added TFA (1.0 ml). The mixture was stirred at r.t. for 1 h. The mixture was concentrated and The crude product was added to a Prep HPLC column and eluted with water/acetonitrile (5%-45%) to give the title compound (6.1 mg, 18.82%) as a white solid. ¹H NMR (400 MHz, CD₃OD) δ (ppm) 8.34-8.22 (s, 1H), 7.20 (dd, J=8.5, 5.4 Hz, 2H), 7.09 (t, J=8.8 Hz, 2H), 4.52-4.40 (m, 2H), 4.15-3.98 (m, 3H), 3.70-3.55 (m, 6H), 3.41-3.33 (m, 1H), 3.09-2.77 (m, 7H), 2.55-2.42 (m, 2H), 2.31-2.19 (m, 1H), 2.09-1.97 (m, 1H), 1.92-1.80 (m, 1H), 1.69 (s, 3H), 1.66 (s, 3H), 1.02 (d, J=6.3 Hz, 3H), 0.96 (d, J=6.3 Hz, 3H). MS: 580.3 (M+H⁺).

Example 78: 1-(4-(4-fluorobenzyl)-8,8-dimethyl-7,8-dihydro-6H-pyrrolo[2,3-e][1,2,3]triazolo[1,5-a]pyridin-6-yl)-2-((2R,5R)-5-methyl-2-(((R)-3-methylmorpholino)methyl)piperazin-1-yl)ethan-1-one (compound 128)

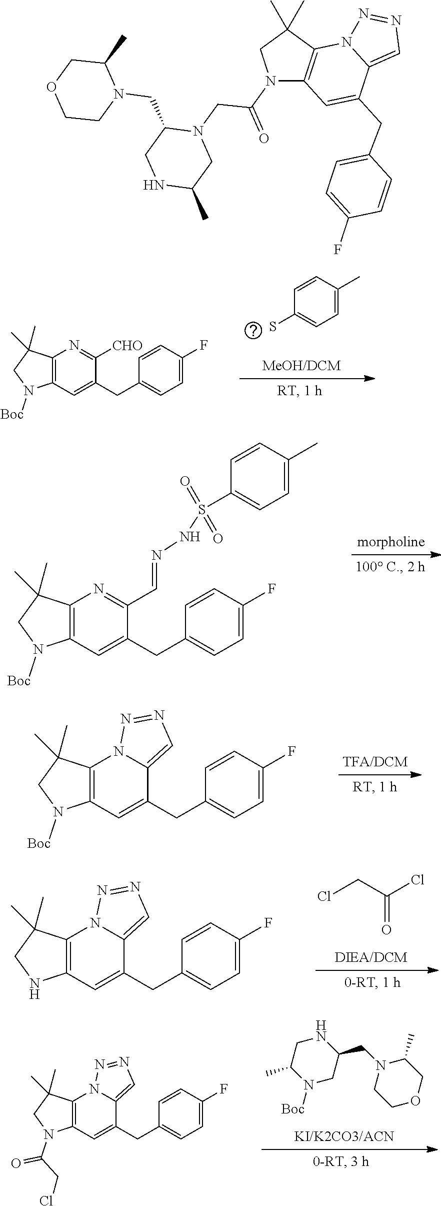

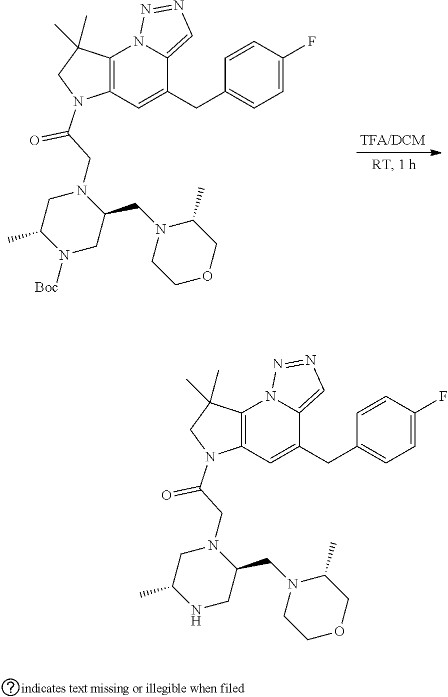 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1cnnn21 ; C[C@@H]1CN(CC(=O)N2CC(C)(C)c3c2cc(Cc2ccc(F)cc2)c2cnnn32)[C@@H](CN2CCOC[C@H]2C)CN1]

Step 1: tert-butyl (E)-6-(4-fluorobenzyl)-3,3-dimethyl-5-((2-tosylhydrazono)methyl)-2,3-dihydro-1H-pyrrolo[3,2-b]pyridine-1-carboxylate

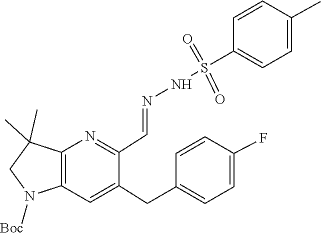

Tert-butyl 6-(4-fluorobenzyl)-5-formyl-3,3-dimethyl-2,3-dihydro-1H-pyrrolo[3,2-b]pyridine-1-carboxylate (0.1 g, 0.260 mmol, 1.0 eq) and 4-methylbenzenesulfonohydrazide (0.048 g, 0.260 mmol, 1.0 eq) were dissolved in MeOH (3 mL) and DCM (3.00 mL) under argon to give a yellow solution. The reaction mixture was stirred at rt for 1 h. The reaction mixture was concentrated to give the product (0.144 g, 100%). MS (ESI) m/z 553.6 [M+H]⁺.

Step 2: tert-butyl 4-(4-fluorobenzyl)-8,8-dimethyl-7,8-dihydro-6H-pyrrolo[2,3-e][1,2,3]triazolo[1,5-a]pyridine-6-carboxylate

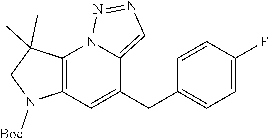 [SMILES: CC(C)(C)OC(=O)N1CC(C)(C)c2c1cc(Cc1ccc(F)cc1)c1cnnn21]

Tert-butyl (E)-6-(4-fluorobenzyl)-3,3-dimethyl-5-((2-tosylhydrazono)methyl)-2,3-dihydro-1H-pyrrolo[3,2-b]pyridine-1-carboxylate (0.144 g, 0.261 mmol, 1.0 eq) was dissolved in morpholine (3 mL) under argon to give a yellow solution. The reaction mixture was stirred at 100° C. for 2 h. H₂O (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 30% to give the product (0.09 g, 87%) as a white solid. MS (ESI) m/z 397.7 [M+H]⁺.

Step 3: 4-(4-fluorobenzyl)-8,8-dimethyl-7,8-dihydro-6H-pyrrolo[2,3-e][1,2,3]triazolo[1,5-a]pyridine

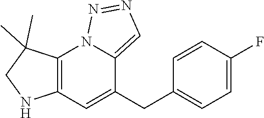 [SMILES: CC1(C)CNc2cc(Cc3ccc(F)cc3)c3cnnn3c21]

Tert-butyl 4-(4-fluorobenzyl)-8,8-dimethyl-7,8-dihydro-6H-pyrrolo[2,3-e][1,2,3]triazolo[1,5-a]pyridine-6-carboxylate (0.09 g, 0.227 mmol, 1.0 eq) was dissolved in TFA (1 mL) and DCM (3.00 mL) under argon to give a yellow solution. The reaction mixture was stirred at rt for 1 h. Saturated NaHCO₃ (30 mL) was added to the reaction mixture followed by extraction with ethyl acetate (30 mL×2). The combined organic layers were dried over Na₂SO₄, filtered and concentrated to give the product (0.067 g, 100%). MS (ESI) m/z 297.8 [M+H]⁺.

Steps 4-6: Following the synthesis of compound 14, compound 128 was obtained as a white solid (6.7 mg). ¹H NMR (400 MHz, DMSO-d⁶) δ (ppm) 8.37 (s, 1H), 8.17 (s, 1H), 7.38 (dd, J=8.3, 5.7 Hz, 2H), 7.14 (t, J=8.8 Hz, 2H), 4.26 (s, 2H), 4.17 (d, J=10.6 Hz, 1H), 3.97 (d, J=10.6 Hz, 1H), 3.76 (dd, J=37.1, 17.0 Hz, 2H), 3.48 (d, J=10.9 Hz, 2H), 3.13 (d, J=11.2 Hz, 1H), 3.08-3.01 (m, 1H), 2.91-2.78 (m, 3H), 2.70-2.64 (m, 2H), 2.36-2.28 (m, 2H), 2.21-2.10 (m, 1H), 1.98-1.88 (m, 2H), 1.78 (d, J=12.8 Hz, 1H), 1.64 (d, J=16.7 Hz, 6H), 0.91 (d, J=5.5 Hz, 6H). MS (ESI) m/z 550.5 [M+H]⁺.

Example 79: 1-(4-(2-amino-4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 129)

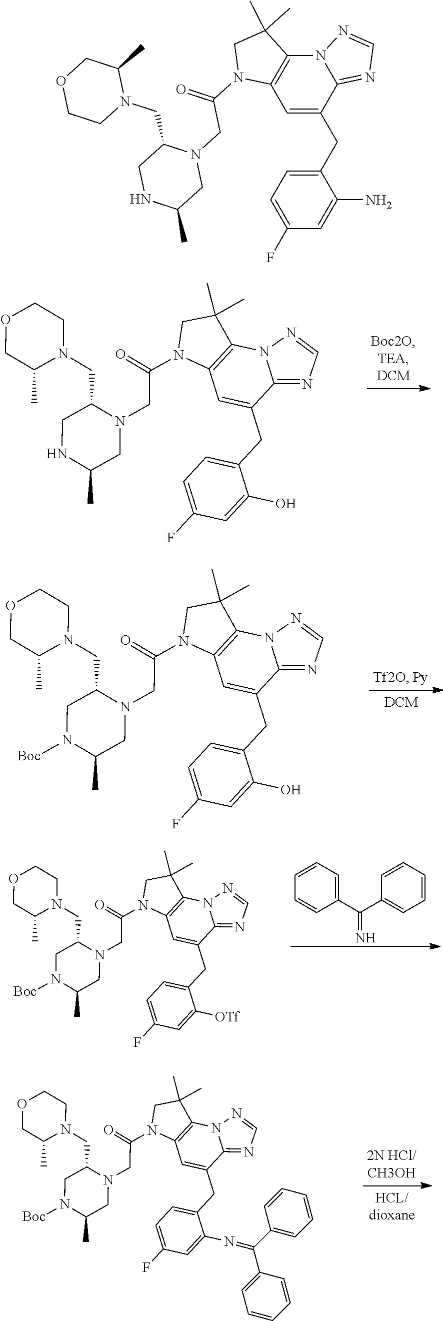

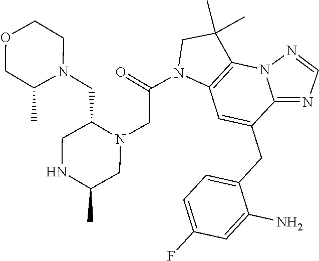 [SMILES: C[C@@H]1CN(CC(=O)N2CC(C)(C)c3c2cc(Cc2ccc(F)cc2N)c2ncnn32)[C@@H](CN2CCOC[C@H]2C)CN1]

Step 1: tert-butyl (2R,5S)-4-(2-(4-(4-fluoro-2-hydroxybenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methyl morpholino)methyl) piperazine-1-carboxylate

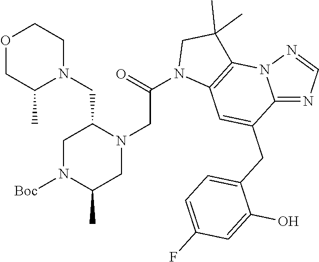 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1O)c1ncnn21]

To a solution of 1-(4-(4-fluoro-2-hydroxybenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (77 mg, 0.136 mmol, 1.0 eq) in DMF (15 mL) were added di-tert-butyl dicarbonate (44.5 mg, 0.204 mmol, 1.5 eq) and triethylamine (41.3 mg, 0.408 mmol, 3.0 eq) at 25° C., then the mixture was stirred at 25° C. for 16 h under N₂, To the mixture was added 2N NaOH(aq) and stirred at rt for 1 h, sat citric acid aq was added, followed by addition of satNaHCO₃ aq. The solution was extracted with ethyl acetate and the organic layer was washed with brine (100 mL), dried over Na₂SO₄, filtered and concentrated, the residue was purified by silica gel flash column chromatography eluting with DCM/CH3OH=20/1 to give the title compound (59 mg, 65.2%) as a yellow solid. MS (ESI) m/z 666.6 [M+H]⁺.

Step 2: tert-butyl (2R,5S)-4-(2-(4-(4-fluoro-2-(((trifluoromethyl)sulfonyl)oxy)benzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

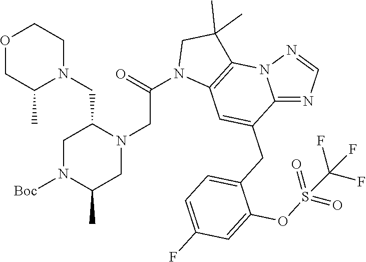 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1OS(=O)(=O)C(F)(F)F)c1ncnn21]

To a solution of tert-butyl (2R,5S)-4-(2-(4-(4-fluoro-2-hydroxybenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (52 mg, 0.078 mmol, 1.0 eq) in CH₂Cl₂ (15 mL) was added pyridine (18.53 mg, 0.234 mmol, 3.0 eq) at 0° C. Trifluoromethanesulfonic anhydride (33.1 mg, 0.117 mmol, 1.5 eq) was added at 0° C. The mixture was stirred at 25° C. for 16 h under N₂. The solution was extracted with ethyl acetate and the organic layer was washed with brine (100 mL), dried over Na₂SO₄, filtered and concentrated, the residue was purified by silica gel flash column chromatography eluting with DCM/CH₃OH=20/1 to give the title compound (50 mg, 80%) as a yellow solid. MS (ESI) m/z 798.5[M+H]+.

Step 3: tert-butyl (2R,5S)-4-(2-(4-(2-((diphenylmethylene)amino)-4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-((( R)-3-methylmorpholino)methyl)piperazine-1-carboxylate

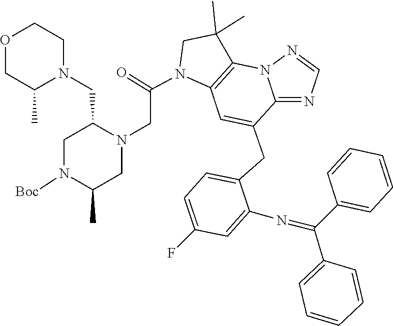 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1N=C(c1ccccc1)c1ccccc1)c1ncnn21]

To a solution of tert-butyl (2R,5S)-4-(2-(4-(4-fluoro-2-(((trifluoromethyl)sulfonyl)oxy)benzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (50 mg, 0.063 mmol, 1.0 eq) in dioxane (15 mL) were added diphenylmethanimine (13.63 mg, 0.075 mmol, 1.2 eq), Pd₂(dba)₃ (5.74 mg, 6.27 μmol, 0.1 eq), BINAP (3.90 mg, 6.27 μmol, 0.1 eq), and Cs₂CO₃ (40.8 mg, 0.125 mmol, 2.0 eq) at 25° C. The mixture was stirred at 100° C. for 16 h under N₂. The solution was extracted with ethyl acetate and the organic layer was washed with brine (100 mL), dried over Na₂SO₄, filtered and concentrated, the residue was purified by silica gel flash column chromatography eluting with DCM/CH₃OH=20/1 to give the title compound (30 mg, 57.7%) as a yellow solid.

Step 4: 1-(4-(2-amino-4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 129)

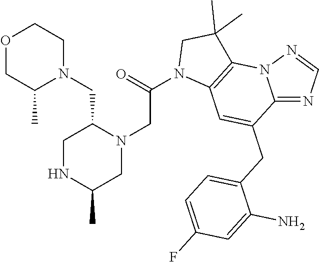 [SMILES: C[C@@H]1CN(CC(=O)N2CC(C)(C)c3c2cc(Cc2ccc(F)cc2N)c2ncnn32)[C@@H](CN2CCOC[C@H]2C)CN1]

To a solution of tert-butyl (2R,5S)-4-(2-(4-(2-((diphenylmethylene)amino)-4-fluorobenzyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (30 mg, 0.036 mmol, 1.0 eq) in MeOH (3 mL) added 2N HCl aq (3 mL) at rt and the mixture was stirred at 25° C. for 1 h. After concentration, 4N HCl/dioxane (5 mL) was added and stirred at rt for 1 h, after the mixture evaporated, the resultant was added NH₃/CH₃OH aq, the solution was concentrated and the residue was purified by pre-HPLC to give the title compound (1.7 mg, 8.32%) as a white solid.

¹H NMR (400 MHz, DMSO-d⁶) δ (ppm) 8.53 (s, 1H), 8.29 (s, 1H), 7.06-6.93 (m, 1H), 6.46-6.34 (m, 1H), 6.31-6.21 (m, 1H), 5.40 (s, 2H), 4.14-4.10 (m, 2H), 3.92 (d, J=10.6 Hz, 1H), 3.80-3.71 (m, 2H), 3.51-3.42 (m, 3H), 3.24-3.14 (m, 2H), 2.92-2.73 (m, 5H), 2.71-2.60 (m, 2H), 2.41-2.25 (m, 1H), 2.20-2.08 (m, 1H), 1.96-1.88 (m, 1H), 1.84-1.74 (m, 1H), 1.59 (d, J=18.4 Hz, 6H), 1.00-0.87 (m, 6H).

Example 80: 4-(4-fluorobenzyl)-1,8,8-trimethyl-6-(2-((2R,5R)-5-methyl-2-(((R)-3-methylmorpholino)methyl)piperazin-1-yl)acetyl)-7,8-dihydro-6H-imidazo[1,5-a]pyrrolo[2,3-e]pyridine-3-carbonitrile (Compound 130)

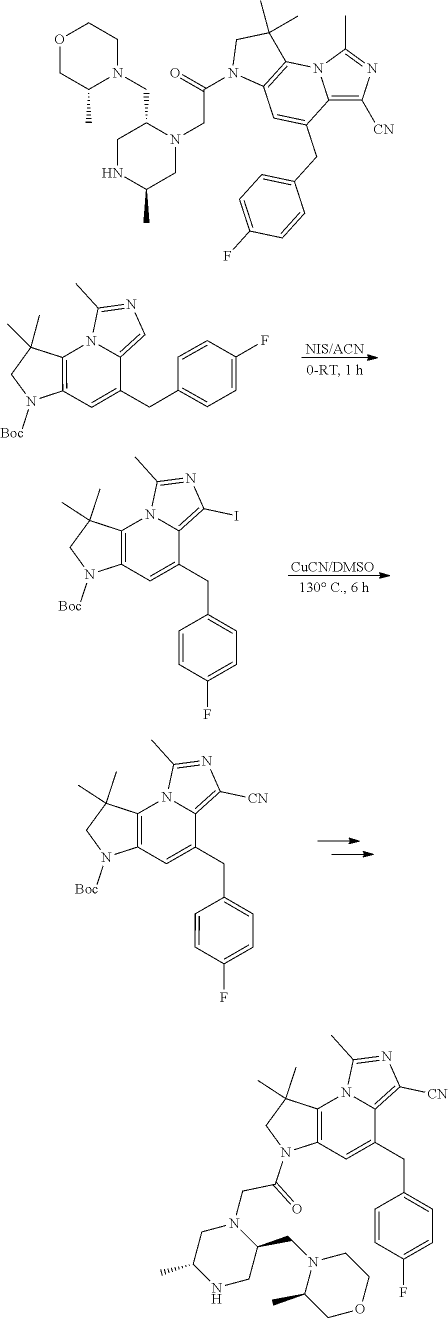

Step 1: tert-butyl 4-(4-fluorobenzyl)-3-iodo-1,8,8-triethyl-7,8-dihydro-6H-imidazo]1,5-a]pyrrolo[2,3-e]pyridine-6-carboxylate

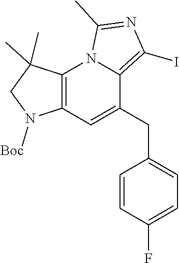 [SMILES: Cc1nc(I)c2c(Cc3ccc(F)cc3)cc3c(n12)C(C)(C)CN3C(=O)OC(C)(C)C]

Tert-butyl 4-(4-fluorobenzyl)-1,8,8-trimethyl-7,8-dihydro-6H-imidazo[1,5-a]pyrrolo[2,3-e]pyridine-6-carboxylate (0.2 g, 0.488 mmol, 1.0 eq) was dissolved in Acetonitrile (10 mL) under argon to give a yellow solution. The reaction mixture was cooled to 0° C. with an ice/water bath. NIS (0.110 g, 0.488 mmol, 1.0 eq) was added to the reaction mixture in one portion. The reaction mixture was stirred at rt for 1 h. Saturated NaCl (30 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 40% to give the product (0.114 g, 43.6%) as a yellow oil. MS (ESI) m/z 536.6 [M+H]⁺.

Step 2: tert-butyl 3-cyano-4-(4-fluorobenzyl)-1,8,8-trimethyl-7,8-dihydro-6H-imidazo[1,5-a]pyrrolo[2,3-e]pyridine-6-carboxylate

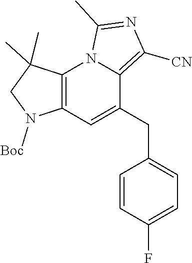 [SMILES: Cc1nc(C#N)c2c(Cc3ccc(F)cc3)cc3c(n12)C(C)(C)CN3C(=O)OC(C)(C)C]

Tert-butyl 4-(4-fluorobenzyl)-3-iodo-1,8,8-trimethyl-7,8-dihydro-6H-imidazo[1,5-a]pyrrolo[2,3-e]pyridine-6-carboxylate (0.114 g, 0.213 mmol, 1.0 eq) and copper(I) cyanide (0.057 g, 0.639 mmol, 3.0 eq) were dissolved in DMSO (3 mL) under argon to give a yellow suspension. The reaction mixture was stirred at 135° C. for 6 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 40% to give the product (0.023 g, 24.86%) as a yellow oil. MS (ESI) m/z 435.6 [M+H]⁺.

Following Steps: Following the synthesis of compound 14, compound 130 was synthesized as a white solid (7.8 mg). ¹H NMR (400 MHz, DMSO-d⁶) δ (ppm) 7.97 (s, 1H), 7.34-7.25 (m, 2H), 7.22-7.13 (m, 2H), 4.44-4.30 (m, 2H), 4.08 (d, J=10.5 Hz, 1H), 3.90-3.77 (m, 2H), 3.62 (d, J=16.4 Hz, 1H), 3.51-3.46 (m, 2H), 3.18 (t, J=9.6 Hz, 1H), 3.00-2.72 (m, 8H), 2.71-2.58 (m, 2H), 2.48-2.43 (m, 1H), 2.37-2.27 (m, 1H), 2.22-2.10 (m, 1H), 1.98-1.88 (m, 1H), 1.77 (d, J=11.1 Hz, 1H), 1.57 (d, J=18.0 Hz, 6H), 0.90 (d, J=6.0 Hz, 6H). MS (ESI) m/z 588.7 [M+H]⁺.

Example 81: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-1H-pyrrolo[2,3-e][1,2,4]triazolo[1,5-a]pyridin-2(6H)-one (Compound 131)

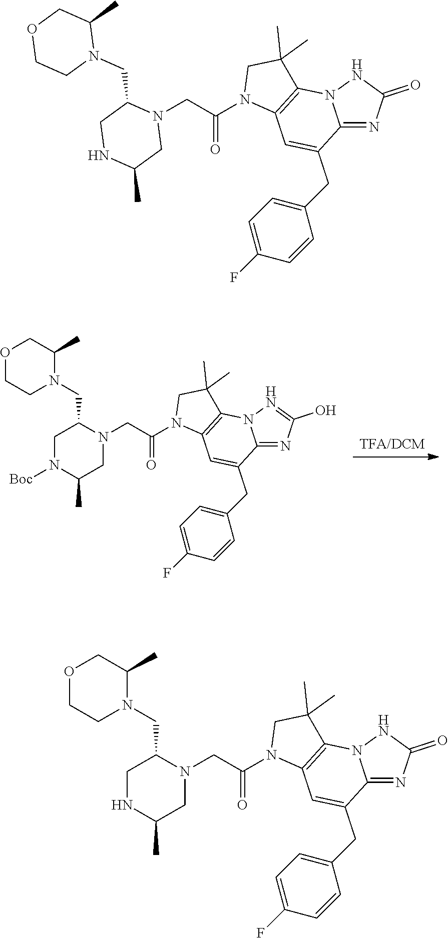 [SMILES: C[C@@H]1CN(CC(=O)N2CC(C)(C)c3c2cc(Cc2ccc(F)cc2)c2nc(=O)[nH]n32)[C@@H](CN2CCOC[C@H]2C)CN1 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(O)nn21 ; C[C@@H]1CN(CC(=O)N2CC(C)(C)c3c2cc(Cc2ccc(F)cc2)c2nc(=O)[nH]n32)[C@@H](CN2CCOC[C@H]2C)CN1]

To a solution of tert-butyl (2R,5S)-4-(2-(4-(4-fluorobenzyl)-2-hydroxy-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate in DCM (2.0 ml) was added TFA (1.0 ml). The mixture was stirred at r.t. for 1 h. The mixture was concentrated and The crude product was added to a Prep HPLC column and was eluted with water/acetonitrile (5%-45%) to give the product as a white solid. MS: 566.4 (M+H⁺).

Example 82: 1-(8,8-dimethyl-4-((2-methylthiazol-5-yl)methyl)-7,8-dihydro-6H-pyrrolo[2,3-e][1,2,4]triazolo[1,5-a]pyridin-6-yl)-2-((2R,5R)-5-methyl-2-(((R)-3-methyl morpholino)methyl)piperazin-1-yl)ethan-1-one (compound 132)

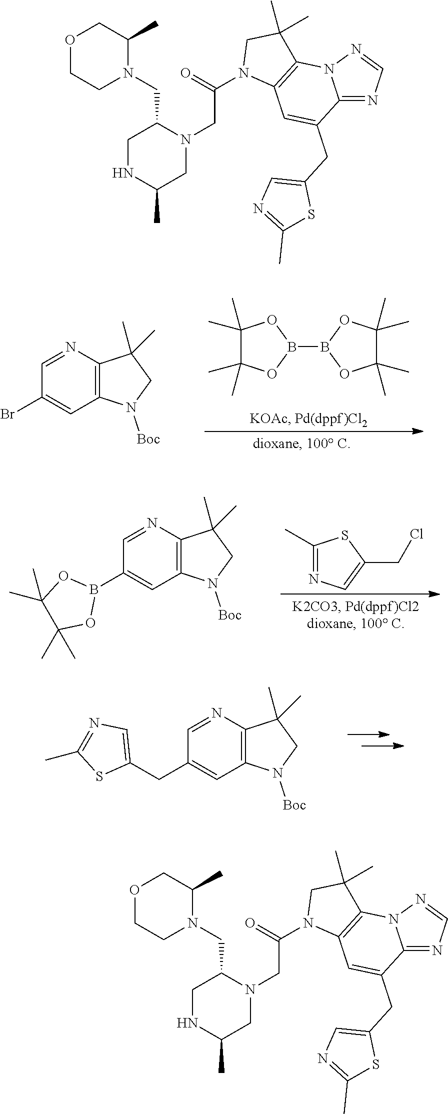

Step 1: tert-butyl 3,3-dimethyl-6-(4,4,5,5-tetramethyl-1,3,2-dioxaborolan-2-yl)-2,3-dihydro-1H-pyrrolo[3,2-b]pyridine-1-carboxylate

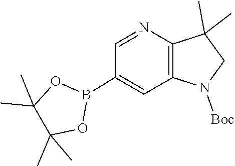

To a solution of tert-butyl 6-bromo-3,3-dimethyl-2,3-dihydro-1H-pyrrolo[3,2-b]pyridine-1-carboxylate (2 g, 6.11 mmol, 1.0 eq) in dioxane (15 mL) were added 4,4,4′,4′,5,5,5′,5′-octamethyl-2,2′-bi(1,3,2-dioxaborolane) (2.328 g, 9.17 mmol, 1.5 eq), PdCl₂(dppf) (0.447 g, 0.611 mmol, 0.1 eq) and potassium acetate (1.800 g, 18.34 mmol, 3.0 eq) at 25° C. The mixture was stirred at 100° C. for 16 h under N₂. Water and ethyl acetate were added. The combined organic layer was washed with brine (100 mL), dried over Na₂SO₄, filtered and concentrated. The residue was purified by silica gel flash column chromatography eluting with PE/EA=3/1 to give the title compound (2.2 g, 96%) as a yellow solid.

Step 2: tert-butyl 3,3-dimethyl-6-((2-methylthiazol-5-yl)methyl)-2,3-dihydro-1H-pyrrolo[3,2-b]pyridine-1-carboxylate

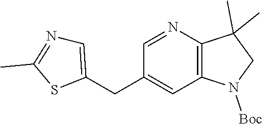

To a solution of tert-butyl 3,3-dimethyl-6-(4,4,5,5-tetramethyl-1,3,2-dioxaborolan-2-yl)-2,3-dihydro-1H-pyrrolo[3,2-b]pyridine-1-carboxylate (759 mg, 2.032 mmol, 1.0 eq) in dioxane (15 mL) were added 5-(chloromethyl)-2-methylthiazole (300 mg, 2.032 mmol, 1.0 eq), PdCl₂(dppf) (149 mg, 0.203 mmol, 0.1 eq), and K₂CO₃ (843 mg, 6.10 mmol, 3.0 eq) at 25° C. The mixture was stirred at 100° C. for 16 h under N₂. Water and ethyl acetate were added. The combined organic layer was washed with brine (100 mL), dried over Na₂SO₄, filtered and concentrated. The residue was purified by silica gel flash column chromatography eluting with PE/EA=3/1 to give the title compound (630 mg, 86%) as a yellow solid. MS (ESI) m/z 361.1[M+H]⁺.

Following Steps: following the synthesis of compound 14, compound 132 was obtained as a white solid (22.1 mg). MS (ESI) m/z 553.7[M+H]⁺. ¹H NMR (400 MHz, DMSO-d⁶) δ (ppm) 8.53 (s, 1H), 8.45 (s, 1H), 7.52 (s, 1H), 4.49 (s, 2H), 4.20-4.10 (m, 1H), 4.02-3.91 (m, 1H), 3.87-3.76 (m, 1H), 3.73-3.62 (m, 1H), 3.53-3.45 (m, 2H), 3.23-3.12 (m, 1H), 3.06-2.96 (m, 1H), 2.95-2.72 (m, 4H), 2.68-2.61 (m, 2H), 2.55-2.52 (m, 3H), 2.38-2.23 (m, 1H), 2.22-2.10 (m, 1H), 2.03-1.88 (m, 1H), 1.84-1.72 (m, 1H), 1.60 (d, J=16.2 Hz, 6H), 0.95-0.85 (m, 6H).

Example 83: 1-(4-((S)-(4-fluorophenyl)(hydroxy)methyl)-8,8-dimethyl-7,8-dihydro-6H-pyrrolo[2,3-e][1,2,4]triazolo[1,5-a]pyridin-6-yl)-2-((2R,5R)-5-meth yl-2-(((R)-3-methylmorpholino)methyl)piperazin-1-yl)ethan-1-one and

1-(4-((R)-(4-fluorophenyl)(hydroxy)methyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 133 and 134)

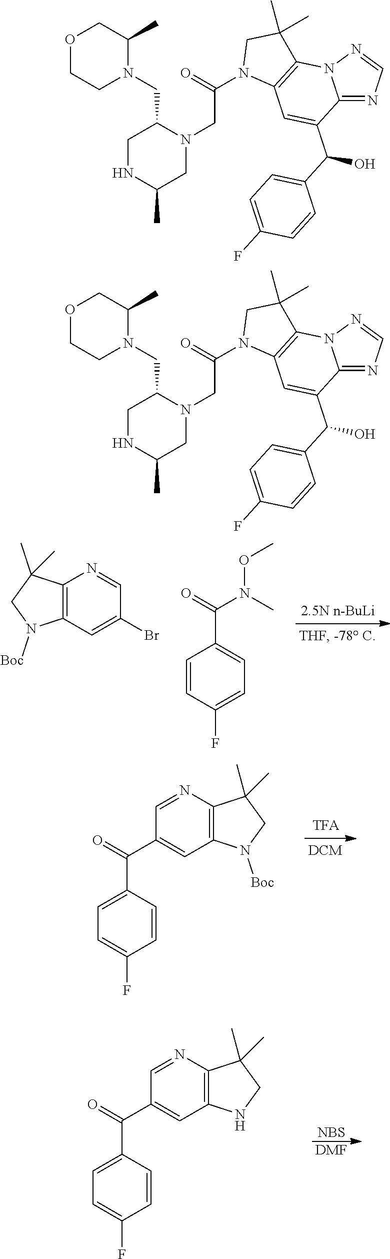

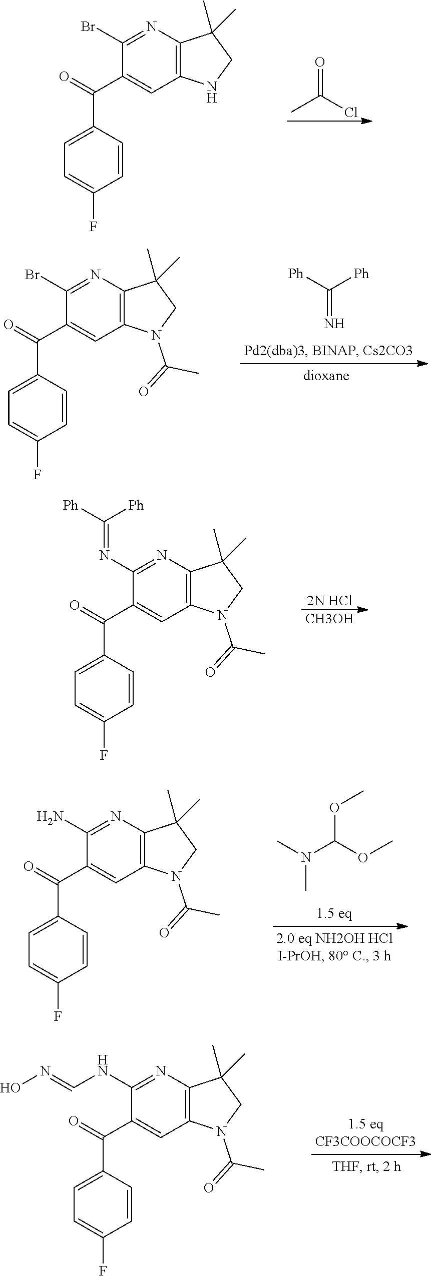

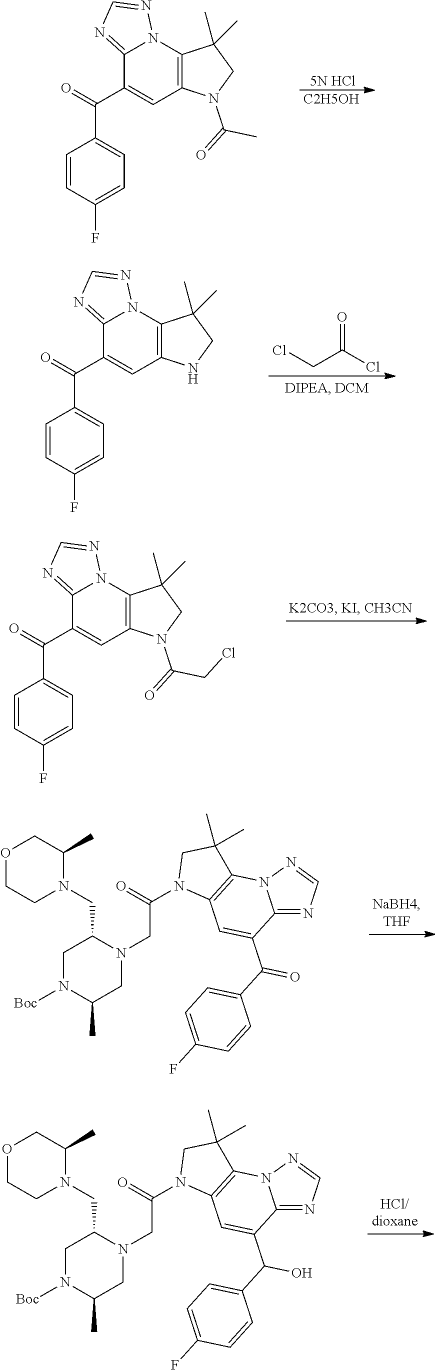

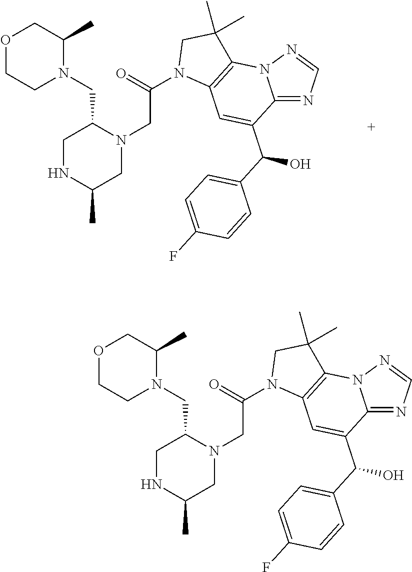 [SMILES: C[C@@H]1CN(CC(=O)N2CC(C)(C)c3c2cc([C@@H](O)c2ccc(F)cc2)c2ncnn32)[C@@H](CN2CCOC[C@H]2C)CN1 ; C[C@@H]1CN(CC(=O)N2CC(C)(C)c3c2cc([C@H](O)c2ccc(F)cc2)c2ncnn32)[C@@H](CN2CCOC[C@H]2C)CN1]

Steps 1-4: 1-(5-bromo-6-(4-fluorobenzoyl)-3,3-dimethyl-2,3-dihydro-1H-pyrrolo[3,2-b]pyridin-1-yl)ethan-1-one

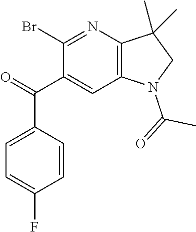 [SMILES: CC(=O)N1CC(C)(C)c2nc(Br)c(C(=O)c3ccc(F)cc3)cc21]

Above intermediate was obtained as a white solid (4.1 g) in a manner similar to that described in compound 166. MS (ESI) m/z 392.2[M+H]+.

Step 5: 1-(5-((diphenylmethylene)amino)-6-(4-fluorobenzoyl)-3,3-dimethyl-2,3-dihydro-1H-pyrrolo[3,2-b]pyridin-1-yl)ethan-1-one

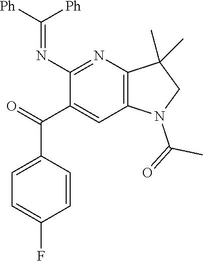 [SMILES: CC(=O)N1CC(C)(C)c2nc(N=C(c3ccccc3)c3ccccc3)c(C(=O)c3ccc(F)cc3)cc21]

To a solution of 1-(5-bromo-6-(4-fluorobenzoyl)-3,3-dimethyl-2,3-dihydro-1H-pyrrolo[3,2-b]pyridin-1-yl)ethan-1-one (300 mg, 0.767 mmol, 1.0 eq) in dioxane (15 mL) were added diphenylmethanimine (167 mg, 0.920 mmol, 1.2 eq), Pd₂(dba)₃ (70.2 mg, 0.077 mmol, 0.1 eq), BINAP (47.7 mg, 0.077 mmol, 0.1 eq), and Cs₂CO₃ (500 mg, 1.534 mmol, 2.0 eq) at 25° C. The mixture was stirred at 100° C. for 16 h under N₂. Water and ethyl acetate were added, and the combined organic layer was washed with brine (100 mL), dried over Na₂SO₄, filtered and concentrated, The residue was purified by silica gel flash column chromatography eluting with PE/EA=3/1 to give the title compound (330 mg, 88%) as a yellow solid. MS (ESI) m/z 492.4 [M+H]⁺.

Step 6: 1-(5-amino-6-(4-fluorobenzoyl)-3,3-dimethyl-2,3-dihydro-1H-pyrrolo[3,2-b]pyridin-1-yl)ethan-1-one

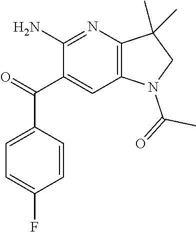 [SMILES: CC(=O)N1CC(C)(C)c2nc(N)c(C(=O)c3ccc(F)cc3)cc21]

To a solution of 1-(5-((diphenylmethylene)amino)-6-(4-fluorobenzoyl)-3,3-dimethyl-2,3-dihydro-1H-pyrrolo[3,2-b]pyridin-1-yl)ethan-1-one (361 mg, 0.734 mmol, 1.0 eq) in MeOH (15 mL) was added 2 N HCl (10 mL) at 25° C. The mixture was stirred at 25° C. for 1 h. Sat NaHCO₃ aq was added. The solution was extracted with ethyl acetate and the organic layer was washed with brine (100 mL), dried over Na₂SO₄, filtered and concentrated. The residue was purified by silica gel flash column chromatography eluting with PE/EA=1/1 to give the title compound (174 mg, 72.4%) as a yellow solid. MS (ESI) m/z 328.3[M+H]⁺.

Steps 7-11: tert-butyl (2R,5S)-4-(2-(4-(4-fluorobenzoyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methy 1) piperazine-1-carboxylate

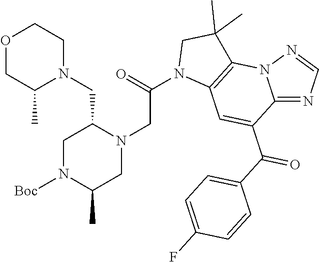 [SMILES: C[C@@H]1COCCN1C[C@H]1CN(C(=O)OC(C)(C)C)[C@H](C)CN1CC(=O)N1CC(C)(C)c2c1cc(C(=O)c1ccc(F)cc1)c1ncnn21]

Following the synthesis of compound 14, above intermediate was obtained as a white solid (226 mg). MS (ESI) m/z 665.4 [M+H]⁺.

Step 12: tert-butyl (2R,5S)-4-(2-(4-((4-fluorophenyl)(hydroxy)methyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methyl morpholino)methyl)piperazine-1-carboxylate

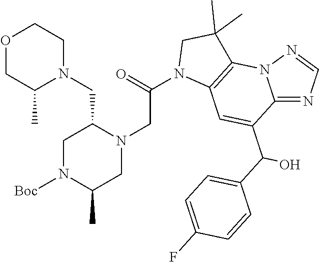 [SMILES: C[C@@H]1COCCN1C[C@H]1CN(C(=O)OC(C)(C)C)[C@H](C)CN1CC(=O)N1CC(C)(C)c2c1cc(C(O)c1ccc(F)cc1)c1ncnn21]

To a solution of tert-butyl (2R,5S)-4-(2-(4-(4-fluorobenzoyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methyl morpholino)methyl)piperazine-1-carboxylate (100 mg, 0.151 mmol, 1.0 eq) in THF (15 mL) was added NaBH₄ (6.84 mg, 0.181 mmol, 1.2) at 0° C. and stirred at rt for 3 h under N₂. Sat NH₄Cl aq and ethyl acetate were added. The combined organic layer was washed with brine (100 mL), dried over Na₂SO₄, filtered and concentrated. The residue was purified by silica gel flash column chromatography eluting with DCM/CH₃OH=50/1 to give the title compound (37 mg, 36.9%) as a yellow solid. MS (ESI) m/z 667.2[M+H]⁺.

Step 13: 1-(4-((4-fluorophenyl)(hydroxy)methyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl) piperazin-1-yl)ethan-1-one (compound 133 and compound 134)

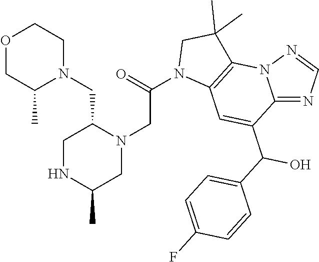 [SMILES: C[C@@H]1CN(CC(=O)N2CC(C)(C)c3c2cc(C(O)c2ccc(F)cc2)c2ncnn32)[C@@H](CN2CCOC[C@H]2C)CN1]

After acidic deprotection and pre-HPLC purification, compound 133 and 134 was obtained separately.

Compound 133: white solid (11.4 mg). MS (ESI) m/z 566.6[M+H]⁺. ¹H NMR (400 MHz, DMSO-d⁶) δ (ppm) 8.70 (s, 1H), 8.46 (s, 1H), 7.54-7.41 (m, 2H), 7.17-6.99 (m, 2H), 6.34-6.13 (m, 2H), 4.09 (d, J=10.6 Hz, 1H), 3.96 (d, J=10.6 Hz, 1H), 3.83-3.66 (m, 2H), 3.54-3.47 (m, 2H), 3.22-3.18 (m, 1H), 3.12-3.04 (m, 1H), 2.95-2.77 (m, 4H), 2.73-2.62 (m, 2H), 2.63-2.50 (m, 1H), 2.40-2.25 (m, 1H), 2.23-2.07 (m, 1H), 2.03-1.88 (m, 1H), 1.79 (d, J=12.3 Hz, 1H), 1.56 (d, J=13.3 Hz, 6H), 0.98-0.85 (m, 6H).

Compound 134: white solid (11.1 mg). MS (ESI) m/z 566.6[M+H]⁺. ¹H NMR (400 MHz, DMSO-d⁶) δ (ppm) 8.74 (s, 1H), 8.48 (s, 1H), 7.56-7.43 (m, 2H), 7.14-7.04 (m, 2H), 6.34-6.19 (m, 2H), 4.17 (d, J=10.6 Hz, 1H), 3.92 (d, J=10.6 Hz, 1H), 3.82 (d, J=16.7 Hz, 1H), 3.69 (d, J=16.5 Hz, 1H), 3.49-3.41 (m, 2H), 3.14-3.01 (m, 2H), 2.94-2.73 (m, 4H), 2.73-2.63 (m, 2H), 2.62-2.54 (m, 1H), 2.41-2.25 (m, 1H), 2.18-2.07 (m, 1H), 2.01-1.86 (m, 1H), 1.77 (d, J=12.7 Hz, 1H), 1.58 (d, J=18.9 Hz, 6H), 0.97-0.85 (m, 6H).

Example 84: 4-(4-fluorobenzyl)-2,8,8-trimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 135)

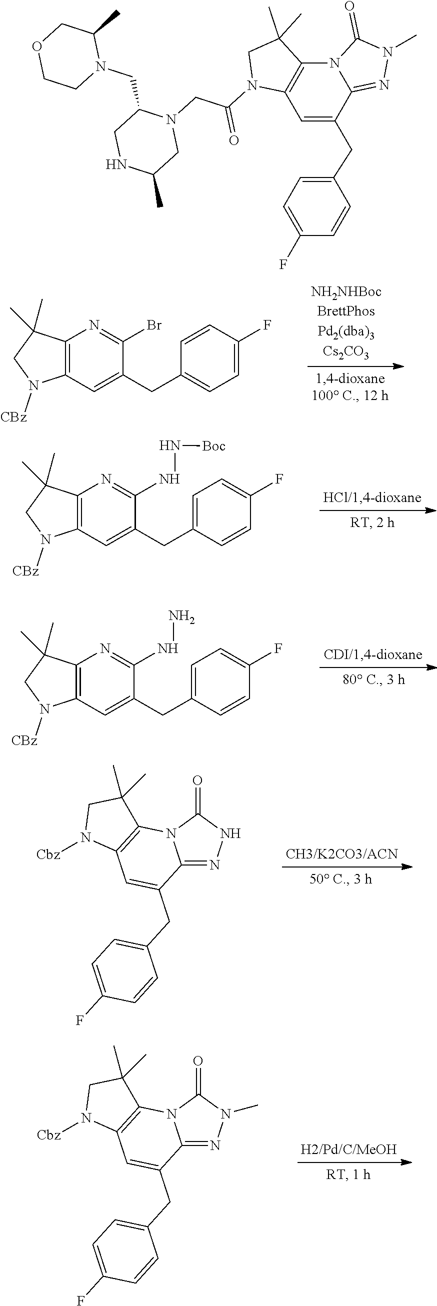

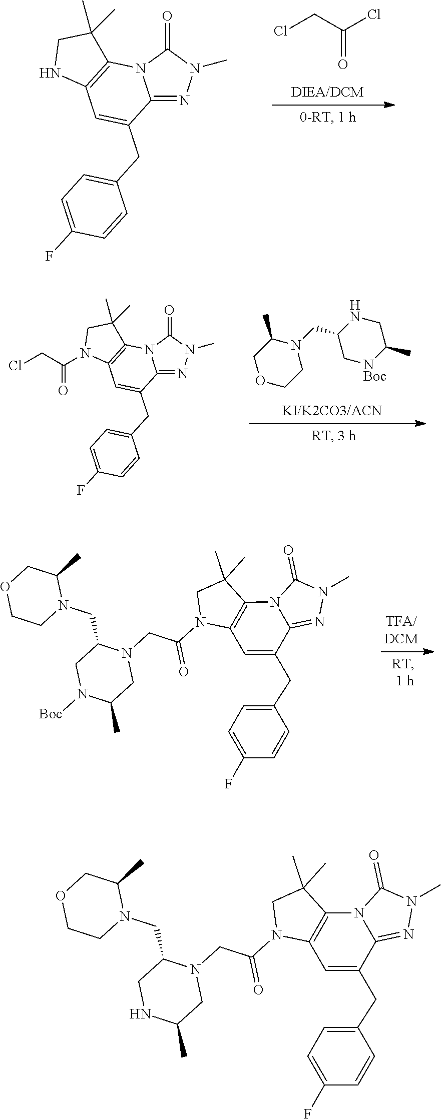

Step 1: benzyl 5-(2-(tert-butoxycarbonyl)hydrazinyl)-6-(4-fluorobenzyl)-3,3-dimethyl-2,3-dihydro-1H-pyrrolo[3,2-b]pyridine-1-carboxylate

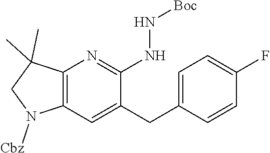 [SMILES: CC(C)(C)OC(=O)NNc1nc2c(cc1Cc1ccc(F)cc1)N(C(=O)OCc1ccccc1)CC2(C)C]

Benzyl 5-bromo-6-(4-fluorobenzyl)-3,3-dimethyl-2,3-dihydro-1H-pyrrolo[3,2-b]pyridine-1-carboxylate (0.6 g, 1.278 mmol, 1.0 eq), Pd₂(dba)₃ (0.117 g, 0.128 mmol, 0.1 eq), Cs₂CO₃ (0.833 g, 2.56 mmol, 2.0 eq), tert-butyl hydrazinecarboxylate (0.338 g, 2.56 mmol, 2.0 eq), and dicyclohexyl(2′,4′,6′-triisopropyl-3,6-dimethoxy-[1,1′-biphenyl]-2-yl)phosphane (0.137 g, 0.256 mmol, 0.2 eq) were dissolved in 1,4-dioxane (20 mL) under argon to give a brown solution. The reaction mixture was stirred at 100° C. for 12 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 20% to give the product (0.7 g, 100%) as a yellow oil. MS (ESI) m/z 521.6 [M+H]⁺.

Step 2: benzyl 6-(4-fluorobenzyl)-5-hydrazinyl-3,3-dimethyl-2,3-dihydro-1H-pyrrolo[3,2-b]pyridine-1-carboxylate

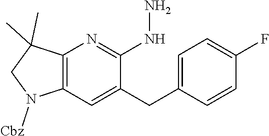 [SMILES: CC1(C)CN(C(=O)OCc2ccccc2)c2cc(Cc3ccc(F)cc3)c(NN)nc21]

Benzyl 5-(2-(tert-butoxycarbonyl)hydrazinyl)-6-(4-fluorobenzyl)-3,3-dimethyl-2,3-dihydro-1H-pyrrolo[3,2-b]pyridine-1-carboxylate (0.7 g, 1.345 mmol, 1.0 eq) and HCl (6 N, 5 mL) were dissolved in 1,4-dioxane (5 mL) under argon to give a brown solution. The reaction mixture was stirred at rt for 2 h. The reaction mixture was concentrated to give the product (0.54 g, 96%) as a yellow solid. MS (ESI) m/z 421.7 [M+H]⁺.

Step 3: benzyl 4-(4-fluorobenzyl)-8,8-dimethyl-1-oxo-1,2,7,8-tetrahydro-6H-pyrrolo[2,3-e][1,2,4]triazolo[4,3-a]pyridine-6-carboxylate

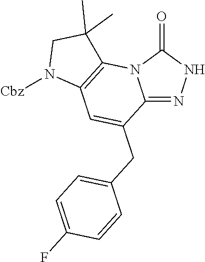 [SMILES: CC1(C)CN(C(=O)OCc2ccccc2)c2cc(Cc3ccc(F)cc3)c3n[nH]c(=O)n3c21]

Benzyl 6-(4-fluorobenzyl)-5-hydrazinyl-3,3-dimethyl-2,3-dihydro-1H-pyrrolo[3,2-b]pyridine-1-carboxylate (0.2 g, 0.476 mmol, 1.0 eq) and CDI (0.386 g, 2.378 mmol, 5.0 eq) were dissolved in 1,4-Dioxane (5 mL) under argon to give a yellow solution. The reaction mixture was stirred at 80° C. for 3 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 60% to give the product (0.096 g, 45.2%) as a color oil. MS (ESI) m/z 447.4 [M+H]⁺.

Step 4: benzyl 4-(4-fluorobenzyl)-2,8,8-trimethyl-1-oxo-1,2,7,8-tetrahydro-6H-pyrrolo[2,3-e][1,2,4]triazolo[4,3-a]pyridine-6-carboxylate

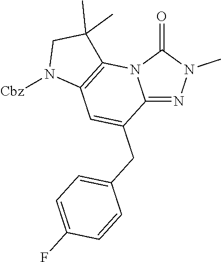 [SMILES: Cn1nc2c(Cc3ccc(F)cc3)cc3c(n2c1=O)C(C)(C)CN3C(=O)OCc1ccccc1]

Benzyl 4-(4-fluorobenzyl)-8,8-dimethyl-1-oxo-1,2,7,8-tetrahydro-6H-pyrrolo[2,3-e][1,2,4]triazolo[4,3-a]pyridine-6-carboxylate (0.116 g, 0.260 mmol, 1.0 eq), K₂CO₃ (0.108 g, 0.779 mmol, 3.0 eq), and iodomethane (0.111 g, 0.779 mmol, 3.0 eq) were dissolved in MeCN (5 mL) under argon to give a yellow suspension. The reaction mixture was stirred at 50° C. for 2 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 25% to give the product (0.095 g, 79%) as a yellow oil. MS (ESI) m/z 461.5 [M+H]⁺.

Step 5: 4-(4-fluorobenzyl)-2,8,8-trimethyl-2,6,7,8-tetrahydro-1H-pyrrolo[2,3-e][1,2,4]triazolo [4,3-a]pyridin-1-one

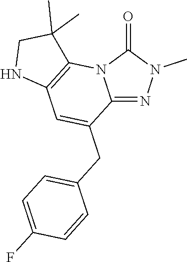 [SMILES: Cn1nc2c(Cc3ccc(F)cc3)cc3c(n2c1=O)C(C)(C)CN3]

Benzyl 4-(4-fluorobenzyl)-2,8,8-trimethyl-1-oxo-1,2,7,8-tetrahydro-6H-pyrrolo[2,3-e][1,2,4]triazolo[4,3-a]pyridine-6-carboxylate (0.095 g, 0.206 mmol, 1.0 eq) and Pd/C (0.040 g, 0.206 mmol, 1.0 eq) were dissolved in MeOH (10 mL) under H₂ to give a black suspension. The reaction mixture was stirred at rt for 1 h. The reaction mixture was filtered through a celite and the filter cake was rinsed with ethyl acetate. The filtrate was concentrated to give the product (0.067 g, 100%). MS (ESI) m/z 327.6 [M+H]⁺.

Steps 6-8: Following the synthesis of compound 14, compound 135 was obtained as a white solid (7.4 mg). ¹H NMR (400 MHz, DMSO-d⁶) δ (ppm) 7.93 (s, 1H), 7.37-7.27 (m, 2H), 7.18-7.09 (m, 2H), 4.02-3.90 (m, 3H), 3.78 (d, J=10.6 Hz, 1H), 3.68 (s, 2H), 3.55 (s, 3H), 3.51-3.47 (m, 2H), 3.20-3.16 (m, 2H), 2.93-2.72 (m, 4H), 2.71-2.63 (m, 2H), 2.40 (t, J=11.5 Hz, 1H), 2.21-2.12 (m, 1H), 2.06-1.90 (m, 2H), 1.80 (d, J=12.4 Hz, 1H), 1.58 (d, J=12.8 Hz, 6H), 0.97 (d, J=6.2 Hz, 3H), 0.89 (d, J=6.2 Hz, 3H). MS (ESI) m/z 580.6 [M+H]⁺.

Example 85: 1-(1-amino-4-(4-fluorobenzyl)-8,8-dimethyl-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (compound 136)

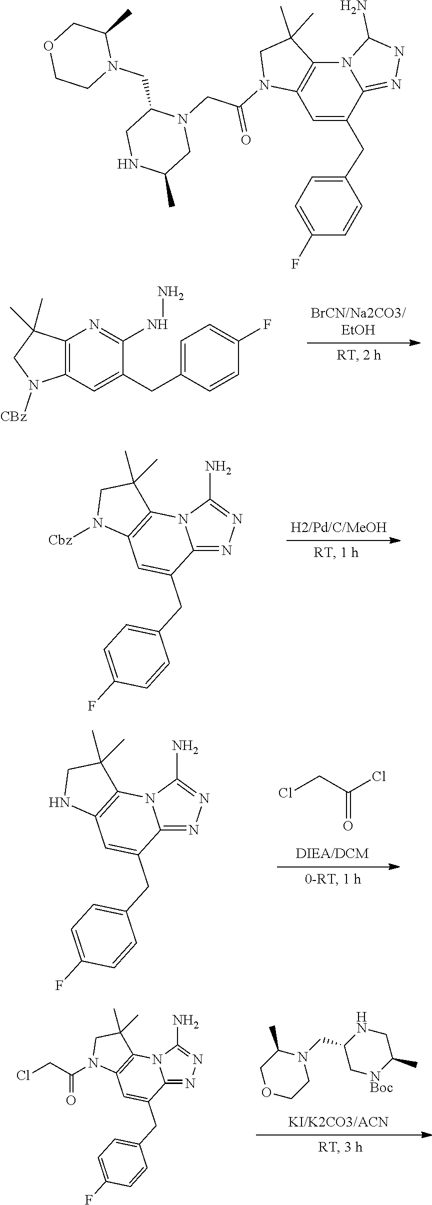

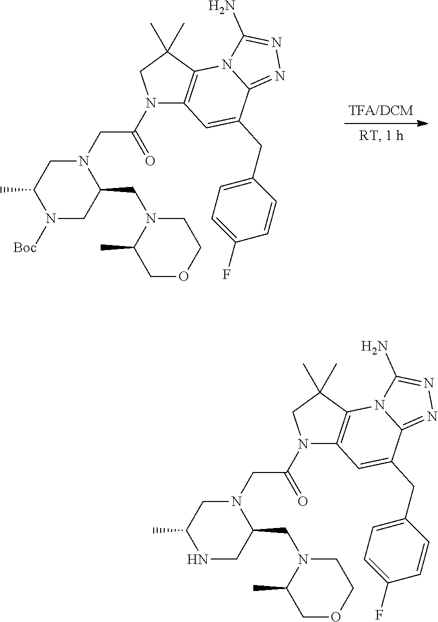 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nnc(N)n21 ; C[C@@H]1CN(CC(=O)N2CC(C)(C)c3c2cc(Cc2ccc(F)cc2)c2nnc(N)n32)[C@@H](CN2CCOC[C@H]2C)CN1]

Step 1: benzyl 1-amino-4-(4-fluorobenzyl)-8,8-dimethyl-7,8-dihydro-6H-pyrrolo[2,3-e][1,2,4]triazolo[4,3-a]pyridine-6-carboxylate

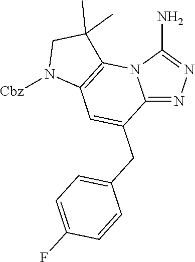 [SMILES: CC1(C)CN(C(=O)OCc2ccccc2)c2cc(Cc3ccc(F)cc3)c3nnc(N)n3c21]

Benzyl 6-(4-fluorobenzyl)-5-hydrazinyl-3,3-dimethyl-2,3-dihydro-1H-pyrrolo[3,2-b]pyridine-1-carboxylate (0.2 g, 0.476 mmol, 1.0 eq), cyanic bromide (0.101 g, 0.951 mmol, 2.0 eq), and Na₂CO₃ (0.151 g, 1.427 mmol, 3.0 eq) were dissolved in EtOH (10 mL) under argon to give a yellow solution. The reaction mixture was stirred at rt for 3 h. Saturated NaCl (10 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.076 g, 35.9%) as a yellow oil. MS (ESI) m/z 446.6 [M+H]⁺.

Steps 2-5: Following the synthesis of compound 135, compound 136 was obtained as a white solid (12.5 mg). ¹H NMR (400 MHz, DMSO-d⁶) δ (ppm) 8.02 (s, 1H), 7.39 (dd, J=8.4, 5.7 Hz, 2H), 7.11 (t, J=8.9 Hz, 2H), 5.34 (s, 2H), 4.17 (q, J=14.8 Hz, 2H), 4.01 (d, J=10.5 Hz, 1H), 3.84-3.71 (m, 2H), 3.60 (d, J=16.6 Hz, 1H), 3.50-3.45 (m, 2H), 3.19-3.13 (m, 1H), 2.99-2.84 (m, 3H), 2.82-2.77 (m, 1H), 2.72 (d, J=9.6 Hz, 1H), 2.61 (d, J=8.9 Hz, 2H), 2.48-2.41 (m, 1H), 2.32-2.24 (m, 1H), 2.21-2.09 (m, 1H), 1.92 (t, J=9.5 Hz, 1H), 1.75 (d, J=12.2 Hz, 1H), 1.60 (d, J=19.2 Hz, 6H), 0.94-0.86 (m, 6H). MS (ESI) m/z 565.8 [M+H]⁺.

Example 86: 1-(4-(4-fluorobenzyl)-2-(2-hydroxypropan-2-yl)-8,8-dimeth yl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 137)

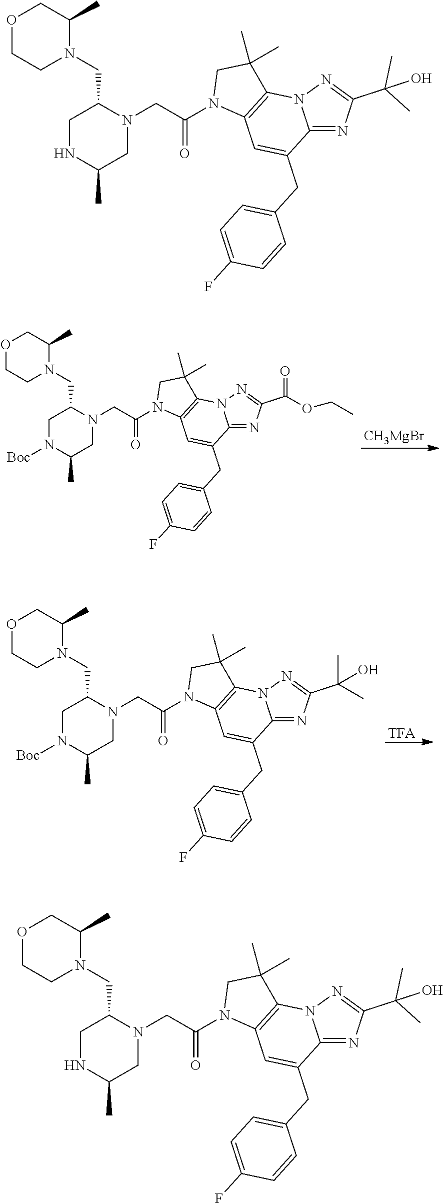

Step 1: tert-butyl (2R,5S)-4-(2-(4-(4-fluorobenzyl)-2-(2-hydroxypropan-2-yl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

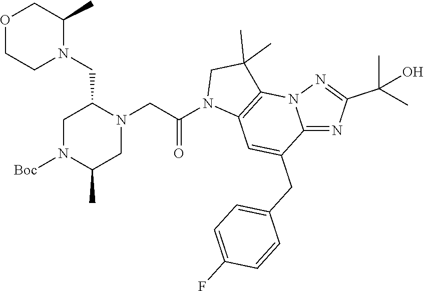 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C(C)(C)O)nn21]

In a flame-dried 100 mL round-bottomed flask, ethyl 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[1,5-a]pyridine-2-carboxylate (270 mg, 0.374 mmol, 1.0 eq) was dissolved in THF (5 mL). Methylmagnesium bromide (223 mg, 1.870 mmol, 5.0 eq) was added to the reaction mixture dropwise over 10 minutes at −78° C. The mixture was stirred for 2 h at −78° C. and overnight at RT. Saturated NH₄Cl (5 ml) was added to the reaction mixture followed by extraction with ethyl acetate (10 ml×3). The combined organic layers were dried over Na₂SO₄, filtered and concentrated to give the title compound (240 mg, 91%) as a light yellow oil. MS (ESI) m/z 708.9 [M+H]⁺.

After acidic deprotection and pre-HPLC purification, compound 137 was obtained as a white solid after lyophilization. MS (ESI) m/z 608.6 [M+H]⁺.

Example 87: 1-(4-(fluoro(4-fluorophenyl)methyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 138)

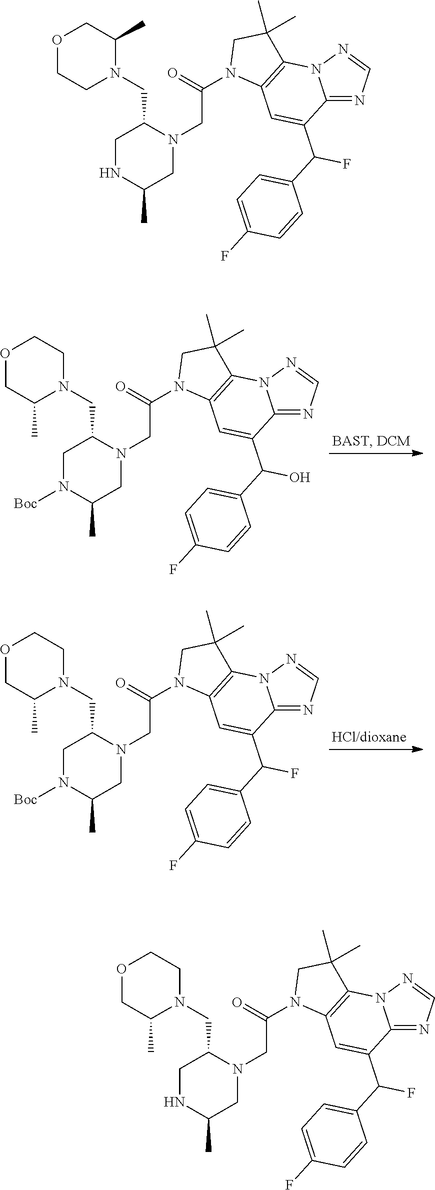

Step 1: tert-butyl (2R,5S)-4-(2-(4-((4-fluorophenyl)(hydroxy)methyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methyl morpholino)methyl)piperazine-1-carboxylate

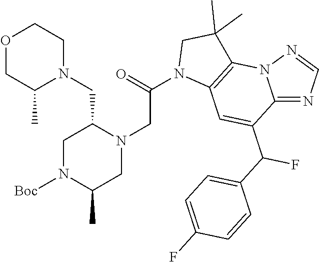 [SMILES: C[C@@H]1COCCN1C[C@H]1CN(C(=O)OC(C)(C)C)[C@H](C)CN1CC(=O)N1CC(C)(C)c2c1cc(C(F)c1ccc(F)cc1)c1ncnn21]

To a solution of tert-butyl (2R,5S)-4-(2-(4-((4-fluorophenyl)(hydroxy)methyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methyl morpholino)methyl)piperazine-1-carboxylate (113 mg, 0.170 mmol, 1.0 eq) in THF (15 mL) was added BAST (75 mg, 0.339 mmol, 2.0 eq) at 0° C. and stirred at rt for 3 h under N₂. To the reaction mixture were added sat NH₄Cl aq and ethyl acetate. The combined organic layer was washed with brine (100 mL), dried over Na₂SO₄, filtered and concentrated. The residue was purified by silica gel flash column chromatography eluting with DCM/CH₃OH=50/1 to give the title compound (100 mg, 88%) as a yellow solid. MS (ESI) m/z 669.0 [M+H]⁺.

Step 2: compound 138 was obtained as a white solid (33.5 mg) after acidic deprotection and pre-HPLC purification. MS (ESI) m/z 568.5 [M+H]⁺. ¹H NMR (400 MHz, DMSO-d⁶) δ (ppm) 8.76-8.59 (m, 1H), 8.53-8.41 (m, 1H), 7.60-7.42 (m, 2H), 7.20 (dd, J=16.3, 7.4 Hz, 2H), 7.13-7.01 (m, 1H), 4.20-4.06 (m, 1H), 4.04-3.88 (m, 1H), 3.86-3.75 (m, 1H), 3.72-3.59 (m, 1H), 3.55-3.42 (m, 2H), 3.23-3.08 (m, 2H), 3.02-2.75 (m, 4H), 2.75-2.66 (m, 1H), 2.67-2.54 (m, 2H), 2.35-2.19 (m, 1H), 2.18-2.05 (m, 1H), 2.00-1.83 (m, 1H), 1.79-1.70 (m, 1H), 1.64-1.52 (m, 6H), 0.95-0.80 (m, 6H).

Example 88: 1-(4-(4-fluorobenzoyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methyl morpholino)methyl) piperazin-1-yl)ethan-1-one (compound 139)

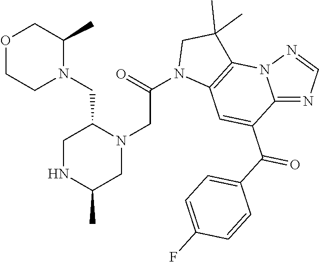 [SMILES: C[C@@H]1CN(CC(=O)N2CC(C)(C)c3c2cc(C(=O)c2ccc(F)cc2)c2ncnn32)[C@@H](CN2CCOC[C@H]2C)CN1]

Compound 139 was obtained as a white solid (13.2 mg). MS (ESI) m/z 564.5 [M+H]⁺. ¹H NMR (400 MHz, DMSO-d⁶) δ (ppm) 8.72 (s, 1H), 8.53 (s, 1H), 7.98-7.81 (m, 2H), 7.46-7.26 (m, 2H), 4.25 (d, J=10.5 Hz, 1H), 4.05 (d, J=10.5 Hz, 1H), 3.92 (d, J=16.5 Hz, 1H), 3.65 (d, J=16.6 Hz, 1H), 3.58-3.50 (m, 2H), 3.27-3.22 (m, 1H), 3.03-2.69 (m, 5H), 2.68-2.58 (m, 2H), 2.45-2.36 (m, 1H), 2.36-2.25 (m, 11H), 2.22-2.13 (m, 11H), 2.02-1.91 (m, 1H), 1.78 (d, J=11.8 Hz, 11H), 1.67 (d, J=13.8 Hz, 6H), 0.97-0.84 (m, 6H).

Example 89: 1-(4-(4-fluorobenzyl)-8,8-dimethyl-2-(tetrahydro-2H-pyran-4-yl)-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 140)

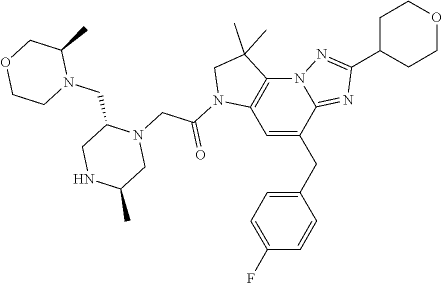 [SMILES: C[C@@H]1CN(CC(=O)N2CC(C)(C)c3c2cc(Cc2ccc(F)cc2)c2nc(C4CCOCC4)nn32)[C@@H](CN2CCOC[C@H]2C)CN1]

Compound 140 was obtained as a white solid (34 mg) in a manner similar to that described in compound 163. ¹H NMR (400 MHz, DMSO-d⁶) δ (ppm) 8.31 (s, 1H), 7.43 (dd, J=8.4, 5.6 Hz, 2H), 7.15 (t, J=8.8 Hz, 2H), 4.33-4.23 (m, 2H), 4.15 (d, J=10.6 Hz, 1H), 4.01-3.90 (m, 3H), 3.85-3.65 (m, 2H), 3.59-3.43 (m, 4H), 3.32-3.29 (m, 2H), 3.24-3.01 (m, 3H), 2.94-2.62 (m, 6H), 2.58-2.54 (m, 1H), 2.37-2.28 (m, 1H), 2.22-2.12 (m, 1H), 2.02-1.84 (m, 5H), 1.61 (d, J=18.6 Hz, 6H), 0.93 (d, J=4.9 Hz, 6H). MS (ESI) m/z 634.73 [M+H]⁺

Example 90: 1-(4-(4-fluorobenzyl)-8,8-dimethyl-2-((methylsulfonyl)meth yl)-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 141)

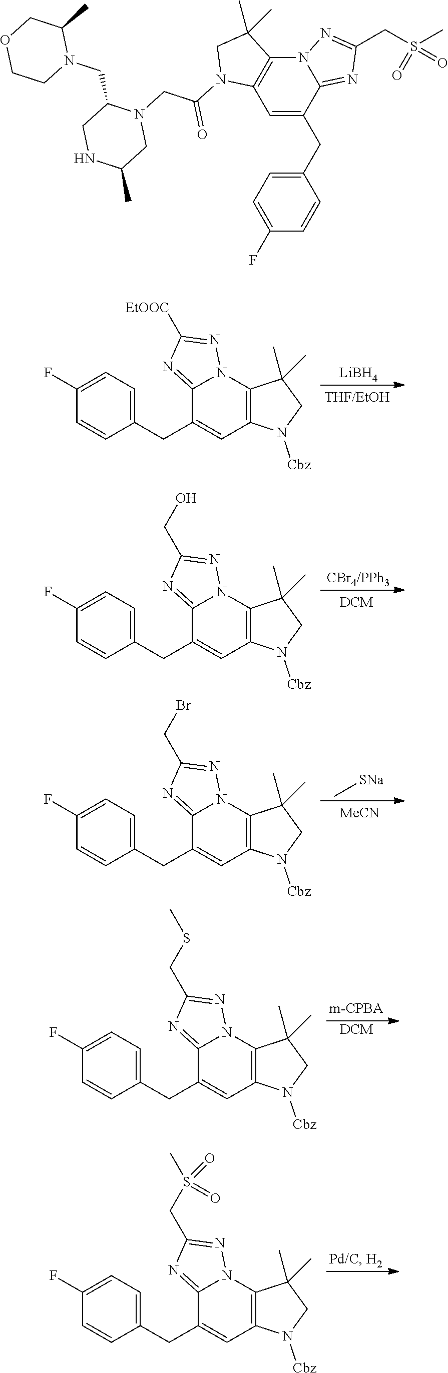

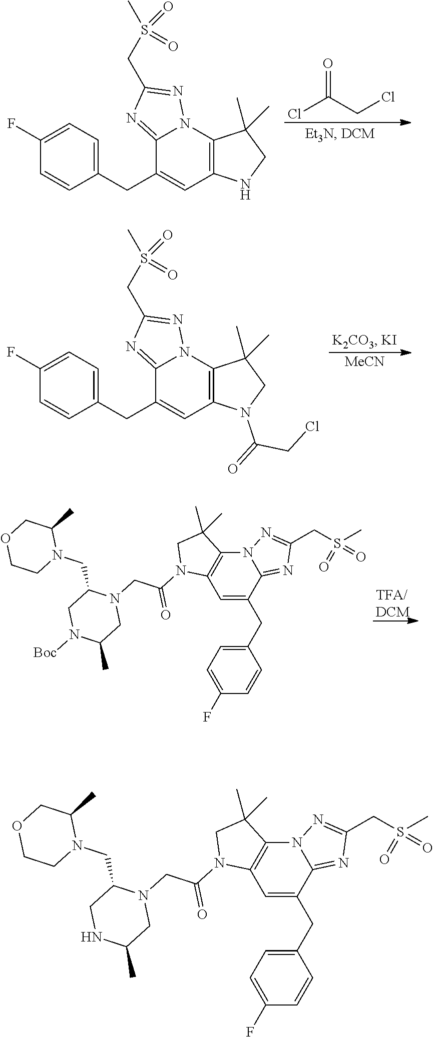

Step 1: benzyl 4-(4-fluorobenzyl)-2-(hydroxymethyl)-8,8-dimethyl-7,8-dihydro-6H-pyrrolo[2,3-e][1,2,4]triazolo[1,5-a]pyridine-6-carboxylate

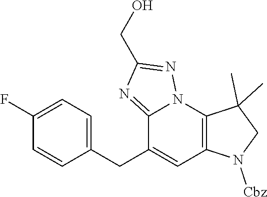 [SMILES: CC1(C)CN(C(=O)OCc2ccccc2)c2cc(Cc3ccc(F)cc3)c3nc(CO)nn3c21]

To a solution of 6-benzyl 2-ethyl 4-(4-fluorobenzyl)-8,8-dimethyl-7,8-dihydro-6H-pyrrolo[2,3-e][1,2,4]triazolo[1,5-a]pyridine-2,6-dicarboxylate (300 mg, 0.597 mmol, 1.0 eq) in THF (10 mL)/ethanol (1 ml) was added LiBH₄ (39.0 mg, 1.791 mmol, 3.0 eq) at rt. The mixture was stirred for 4 h. the mixture was quenched with water, extracted with EA (twice). the organic layer was washed with brine, dried over Na₂SO₄ and concentrated to give the title compound (270 mg, 98%) as a yellow solid. MS (ESI) m/z 461.89 [M+H]⁺

Step 2: benzyl 2-(bromomethyl)-4-(4-fluorobenzyl)-8,8-dimethyl-7,8-dihydro-6H-pyrrolo[2,3-e][1,2,4]triazolo[1,5-a]pyridine-6-carboxylate

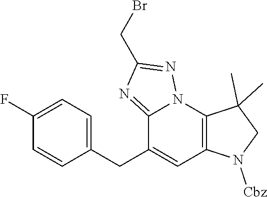 [SMILES: CC1(C)CN(C(=O)OCc2ccccc2)c2cc(Cc3ccc(F)cc3)c3nc(CBr)nn3c21]

To a mixture of benzyl 4-(4-fluorobenzyl)-2-(hydroxymethyl)-8,8-dimethyl-7,8-dihydro-6H-pyrrolo[2,3-e][1,2,4]triazolo[1,5-a]pyridine-6-carboxylate (200 mg, 0.434 mmol, 1.0 eq) and Ph₃P (159 mg, 0.608 mmol, 1.4 eq) in DCM (5 mL) was added CBr₄ (173 mg, 0.521 mmol, 1.2 eq) at 0-10° C. The reaction mixture was stirred for 3 h. The mixture was concentrated to give a yellow oil, which was purified by column eluted with 20% EA/PE to give the title compound (140 mg, 61.6%) as a yellow oil. MS (ESI) m/z 524.81 [M+H]⁺

Step 3: benzyl 4-(4-fluorobenzyl)-8,8-dimethyl-2-((methylthio)methyl)-7,8-dihydro-6H-pyrrolo[2,3-e][1,2,4]triazolo[1,5-a]pyridine-6-carboxylate

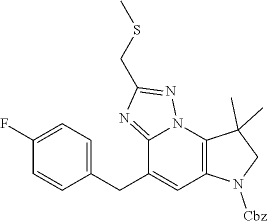 [SMILES: CSCc1nc2c(Cc3ccc(F)cc3)cc3c(n2n1)C(C)(C)CN3C(=O)OCc1ccccc1]

A mixture of benzyl 2-(bromomethyl)-4-(4-fluorobenzyl)-8,8-dimethyl-7,8-dihydro-6H-pyrrolo[2,3-e][1,2,4]triazolo[1,5-a]pyridine-6-carboxylate (140 mg, 0.267 mmol, 1.0 eq) and sodium methanethiolate (187 mg, 2.67 mmol, 10 eq) in acetonitrile (10 mL) was stirred at rt for 0.5 h. The mixture was diluted with water, extracted with ethyl acetate (twice). The organic layer was washed with brine, dried over Na₂SO₄ and concentrated to give the title compound (130 mg, 99%) as a yellow oil. MS (ESI) m/z 491.89 [M+H]⁺

Step 4: benzyl 4-(4-fluorobenzyl)-8,8-dimethyl-2-((methylsulfonyl)methyl)-7,8-dihydro-6H-pyrrolo[2,3-e][1,2,4]triazolo[1,5-a]pyridine-6-carboxylate

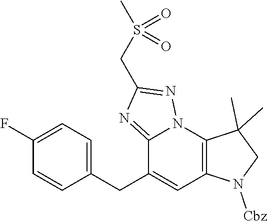 [SMILES: CC1(C)CN(C(=O)OCc2ccccc2)c2cc(Cc3ccc(F)cc3)c3nc(CS(C)(=O)=O)nn3c21]

A mixture of benzyl 4-(4-fluorobenzyl)-8,8-dimethyl-2-((methylthio)methyl)-7,8-dihydro-6H-pyrrolo[2,3-e][1,2,4]triazolo[1,5-a]pyridine-6-carboxylate (130 mg, 0.265 mmol, 1.0 eq) and m-CPBA (137 mg, 0.795 mmol, 3.0 eq) was stirred at 0-10° C. for 1 h. The mixture was quenched with NaHCO₃ (aq), extracted with DCM. the organic layer was washed with brine, dried over Na₂SO₄ and concentrated to give a yellow oil, which was purified by column eluted with 50% EA/PE to give the title compound (45 mg, 32.5%) as a white solid. MS (ESI) m/z 523.83 [M+H]⁺

Step 4: 4-(4-fluorobenzyl)-8,8-dimethyl-2-((methylsulfonyl)methyl)-7,8-dihydro-6H-pyrrolo[2,3-e][1,2,4]triazolo[1,5-a]pyridine

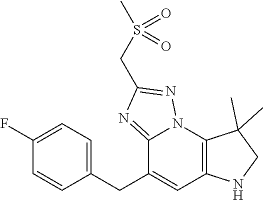 [SMILES: CC1(C)CNc2cc(Cc3ccc(F)cc3)c3nc(CS(C)(=O)=O)nn3c21]

A mixture of benzyl 4-(4-fluorobenzyl)-8,8-dimethyl-2-((methylsulfonyl)methyl)-7,8-dihydro-6H-pyrrolo[2,3-e][1,2,4]triazolo[1,5-a]pyridine-6-carboxylate (45 mg, 0.086 mmol, 1.0 eq) and Pd/C (9 mg, 20%) in THF (10 mL) was degassed and purged with H₂ for three times. The reaction mixture was stirred at 20-25° C. for 4 h. The mixture was filtered and the filtrate was concentrated to give the title compound (30 mg, 90%) as a yellow oil. MS (ESI) m/z 389.77 [M+H]⁺

Following steps: compound 141 was obtained (14 mg, 40.5%) as a white solid in a manner similar to that described in compound 14. ¹H NMR (400 MHz, DMSO-d⁶) δ (ppm) 8.41 (s, 1H), 7.42 (dd, J=8.4, 5.7 Hz, 2H), 7.15 (t, J=8.8 Hz, 2H), 4.87 (s, 2H), 4.39-4.27 (m, 2H), 4.18 (d, J=10.6 Hz, 1H), 3.98 (d, J=10.6 Hz, 1H), 3.90-3.80 (m, 1H), 3.76-3.63 (m, 1H), 3.55-3.47 (m, 2H), 3.31-3.30 (m, 1H), 3.25 (s, 3H), 3.22-3.15 (m, 1H), 3.11-2.63 (m, 8H), 2.42-2.30 (m, 1H), 2.23-2.14 (m, 1H), 2.01-1.92 (m, 1H), 1.86-1.75 (m, 1H), 1.62 (d, J=15.4 Hz, 6H), 0.98-0.87 (m, 6H). MS (ESI) m/z 642.61 [M+H]⁺

Example 91: 1-(2-(difluoromethyl)-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 142)

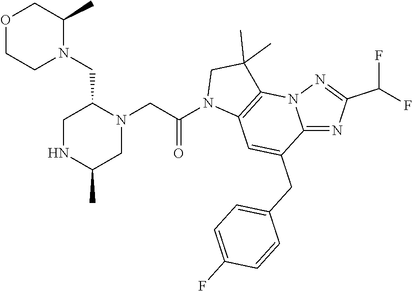 [SMILES: C[C@@H]1CN(CC(=O)N2CC(C)(C)c3c2cc(Cc2ccc(F)cc2)c2nc(C(F)F)nn32)[C@@H](CN2CCOC[C@H]2C)CN1]

MS (ESI) m/z 601.1 [M+H]⁺. ¹H NMR (400 MHz, DMSO-d⁶) δ (ppm) 8.48 (s, 1H), 7.41 (dd, J=8.4, 5.5 Hz, 2H), 7.36 (t, J=53.0 Hz, 1H), 7.16 (t, J=8.7 Hz, 2H), 4.36 (d, J=3.3 Hz, 2H), 4.20 (d, J=10.6 Hz, 1H), 4.00 (d, J=10.5 Hz, 1H), 3.87 (d, J=16.7 Hz, 1H), 3.68 (d, J=16.6 Hz, 1H), 3.50 (dd, J=11.1, 3.2 Hz, 2H), 3.18 (t, J=10.3 Hz, 1H), 3.04-2.80 (m, 3H), 2.74 (dd, J=11.9, 2.8 Hz, 1H), 2.65 (d, J=9.6 Hz, 2H), 2.57-2.54 (m, 2H), 2.51-2.41 (m, 1H), 2.32 (t, J=11.1 Hz, 1H), 2.18 (s, 1H), 1.95 (t, J=10.4 Hz, 1H), 1.79 (d, J=13.0 Hz, 1H), 1.65 (s, 3H), 1.61 (s, 3H), 0.92 (dd, J=9.1, 6.1 Hz, 6H).

Example 92: 1-(4-(4-fluorobenzyl)-8,8-dimethyl-2-morpholino-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 143)

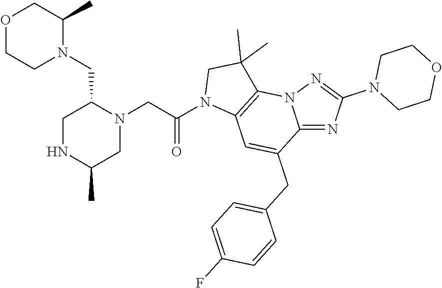 [SMILES: C[C@@H]1CN(CC(=O)N2CC(C)(C)c3c2cc(Cc2ccc(F)cc2)c2nc(N4CCOCC4)nn32)[C@@H](CN2CCOC[C@H]2C)CN1]

Following the synthesis of compound 195, compound 143 was obtained as a white solid MS (ESI) m/z 635.6 [M+H]⁺.

Example 93: 1-(4-(4-fluorobenzyl)-2-(fluoromethyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 144)

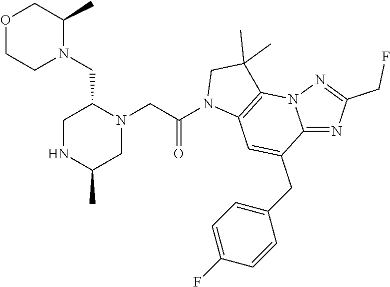 [SMILES: C[C@@H]1CN(CC(=O)N2CC(C)(C)c3c2cc(Cc2ccc(F)cc2)c2nc(CF)nn32)[C@@H](CN2CCOC[C@H]2C)CN1]

Compound 144 was obtained as a white solid after lyophilization in a manner similar to that described in compound 141. MS (ESI) m/z 582.5[M+H]⁺. ¹H NMR (400 MHz, DMSO-d⁶) δ (ppm) 8.42 (s, 1H), 7.45-7.37 (m, 2H), 7.20-7.11 (m, 2H), 5.67 (d, J=47.3 Hz, 2H), 4.34 (d, J=3.4 Hz, 2H), 4.19 (d, J=10.5 Hz, 1H), 3.99 (d, J=10.6 Hz, 1H), 3.86 (d, J=16.7 Hz, 1H), 3.68 (d, J=16.6 Hz, 1H), 3.50 (d, J=11.2 Hz, 2H), 3.18 (t, J=10.3 Hz, 1H), 3.02 (s, 2H), 2.96-2.73 (m, 4H), 2.72-2.61 (m, 2H), 2.51-2.44 (m, 1H), 2.37-2.30 (m, 1H), 2.18 (s, 1H), 2.01-1.91 (m, 1H), 1.80 (d, J=12.9 Hz, 1H), 1.65 (s, 3H), 1.61 (s, 3H), 0.93 (dd, J=6.2, 4.5 Hz, 6H).

Example 94: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[4,3-a]pyridine-1-carboxamide (compound 145)

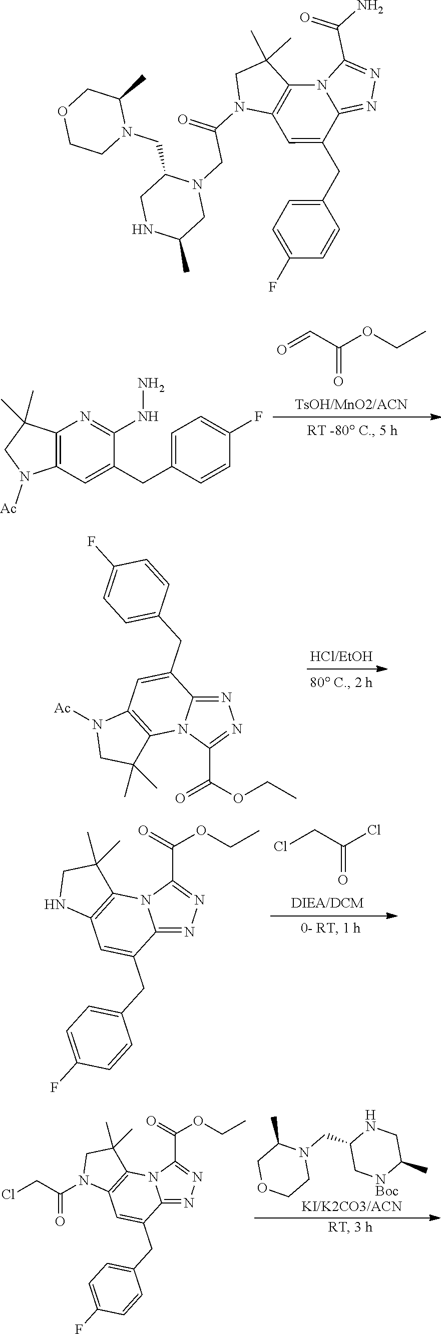

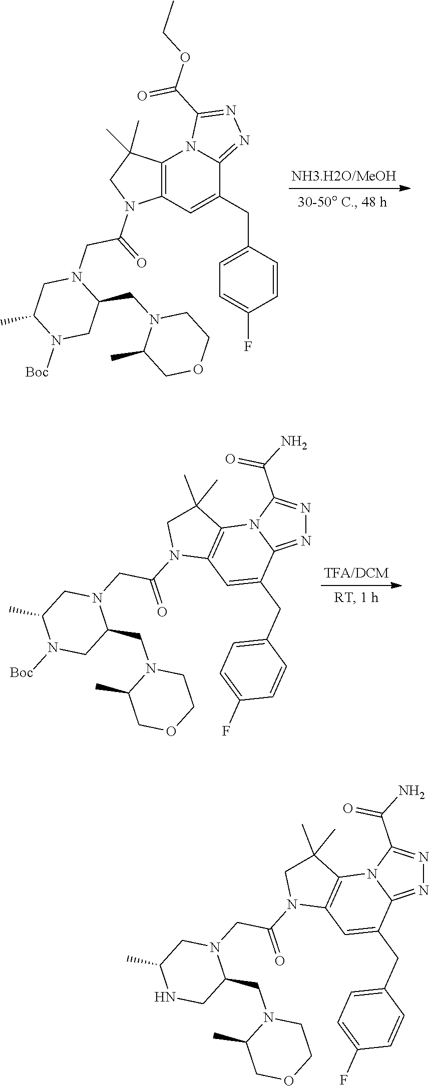 [SMILES: CCOC(=O)c1nnc2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nnc(C(N)=O)n21 ; C[C@@H]1CN(CC(=O)N2CC(C)(C)c3c2cc(Cc2ccc(F)cc2)c2nnc(C(N)=O)n32)[C@@H](CN2CCOC[C@H]2C)CN1]

Step 1: ethyl 6-acetyl-4-(4-fluorobenzyl)-8,8-dimethyl-7,8-dihydro-6H-pyrrolo[2,3-e][1,2,4]triazolo[4,3-a]pyridine-1-carboxylate

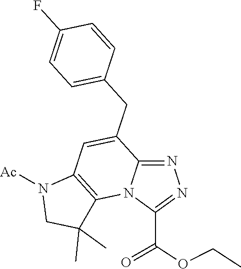 [SMILES: CCOC(=O)c1nnc2c(Cc3ccc(F)cc3)cc3c(n12)C(C)(C)CN3C(C)=O]

1-(6-(4-fluorobenzyl)-5-hydrazinyl-3,3-dimethyl-2,3-dihydro-1H-pyrrolo[3,2-b]pyridin-1-yl)ethan-1-one (0.3 g, 0.914 mmol, 1.0 eq), ethyl 2-oxoacetate (0.280 g, 1.370 mmol, 1.5 eq), and Ts-OH (0.261 g, 1.370 mmol, 1.5 eq) were dissolved in acetonitrile (10 mL) under argon to give a yellow solution. The reaction mixture was stirred at rt for 1 h. Manganese dioxide (0.318 g, 3.65 mmol, 4.0 eq) was added to the reaction mixture in one portion. The reaction mixture was stirred at 80° C. for 4 h. The crude product was added to a silica gel column and was eluted with ethyl acetate/hexane from 0% to 80% to give the product (0.105 g, 28.0%) as a yellow oil. MS (ESI) m/z 411.4 [M+H]⁺.

Step 2: ethyl 4-(4-fluorobenzyl)-8,8-dimethyl-7,8-dihydro-6H-pyrrolo[2,3-e][1,2,4]triazolo[4,3-a]pyridine-1-carboxylate

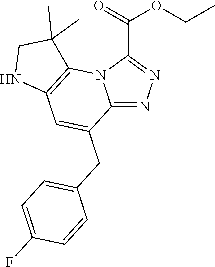 [SMILES: CCOC(=O)c1nnc2c(Cc3ccc(F)cc3)cc3c(n12)C(C)(C)CN3]

Ethyl 6-acetyl-4-(4-fluorobenzyl)-8,8-dimethyl-7,8-dihydro-6H-pyrrolo[2,3-e][1,2,4]triazolo[4,3-a]pyridine-1-carboxylate (0.105 g, 0.256 mmol, 1.0 eq) and HCl (6 N, 3 mL) were dissolved in EtOH (6 mL) under argon to give a yellow solution. The reaction mixture was stirred at 80° C. for 2 h. Saturated NaHCO₃ (30 mL) was added to the reaction mixture followed by extraction with ethyl acetate (30 mL×2). The combined organic layers were dried over Na₂SO₄, filtered and concentrated to give the product (0.094 g, 100%) as a yellow oil. MS (ESI) m/z 369.6 [M+H]⁺.

Step 3: ethyl 6-(2-chloroacetyl)-4-(4-fluorobenzyl)-8,8-dimethyl-7,8-dihydro-6H-pyrrolo[2,3-e][1,2,4]triazolo[4,3-a]pyridine-1-carboxylate

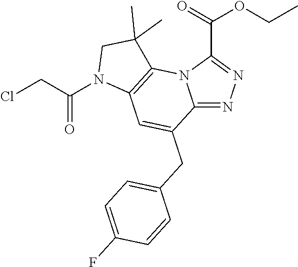 [SMILES: CCOC(=O)c1nnc2c(Cc3ccc(F)cc3)cc3c(n12)C(C)(C)CN3C(=O)CCl]

Ethyl 4-(4-fluorobenzyl)-8,8-dimethyl-7,8-dihydro-6H-pyrrolo[2,3-e][1,2,4]triazolo[4,3-a]pyridine-1-carboxylate (0.094 g, 0.255 mmol, 1.0 eq), 2-chloroacetyl chloride (0.043 g, 0.383 mmol, 1.5 eq), and DIEA (0.099 g, 0.765 mmol, 3.0 eq) were dissolved in DCM (10 mL) under argon to give a yellow solution. The reaction mixture was stirred at rt for 1 h. Saturated NaHCO₃ (20 mL) was added to the reaction mixture followed by extraction with dichloromethane (30 mL×2). The combined organic layers were dried over Na₂SO₄, filtered and concentrated. The crude product was added to a silica gel column and was eluted with ethyl acetate/hexane from 0% to 50% to give the product (0.078 g, 68.7%) as a color oil. MS (ESI) m/z 445.4 [M+H]⁺.

Step 4: ethyl 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[4,3-a]pyridine-1-carboxylate

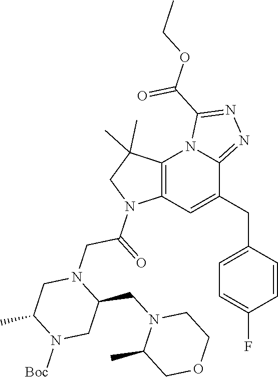 [SMILES: CCOC(=O)c1nnc2c(Cc3ccc(F)cc3)cc3c(n12)C(C)(C)CN3C(=O)CN1C[C@@H](C)N(C(=O)OC(C)(C)C)C[C@@H]1CN1CCOC[C@H]1C]

Ethyl 6-(2-chloroacetyl)-4-(4-fluorobenzyl)-8,8-dimethyl-7,8-dihydro-6H-pyrrolo[2,3-e][1,2,4]triazolo[4,3-a]pyridine-1-carboxylate (0.078 g, 0.175 mmol, 1.0 eq), tert-butyl (2R,5S)-2-methyl-5-(((R)-3-methylmorpholino)methyl)piperazine-1-carboxylate (0.082 g, 0.263 mmol, 1.5 eq), K₂CO₃ (0.073 g, 0.526 mmol, 3 eq), and KI (0.044 g, 0.263 mmol, 1.5 eq) were dissolved in Acetonitrile (10 mL) under argon to give a yellow suspension. The reaction mixture was stirred at rt for 5 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.112 g, 88%) as a yellow oil. MS (ESI) m/z 722.4 [M+H]⁺.

Step 5: tert-butyl (2R,5S)-4-(2-(1-carbamoyl-4-(4-fluorobenzyl)-8,8-dimethyl-7,8-dihydro-6H-pyrrolo[2,3-e][1,2,4]triazolo[4,3-a]pyridin-6-yl)-2-oxoethyl)-2-methyl-5-(((R)-3-methylmorpholino)methyl)piperazine-1-carboxylate

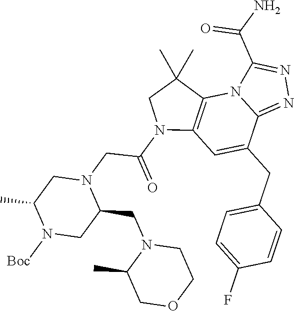 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nnc(C(N)=O)n21]

Ethyl 6-(2-((2S,5R)-4-(tert-butoxycarbonyl)-5-methyl-2-(((R)-3-methylmorpholino)methyl) piperazin-1-yl)acetyl)-4-(4-fluorobenzyl)-8,8-dimethyl-7,8-dihydro-6H-pyrrolo[2,3-e][1,2,4]triazolo[4,3-a]pyridine-1-carboxylate (0.112 g, 0.155 mmol, 1.0 eq) and ammonium hydroxide (2 mL) were dissolved in MeOH (4 mL) under argon to give a yellow solution. The reaction mixture was stirred at 55° C. for 16 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 5% to give the product (0.084 g, 78%) as a yellow oil. MS (ESI) m/z 693.6 [M+H]⁺.

Step 6: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[4,3-a]pyridine-1-carboxamide

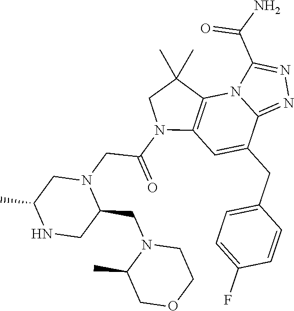 [SMILES: C[C@@H]1CN(CC(=O)N2CC(C)(C)c3c2cc(Cc2ccc(F)cc2)c2nnc(C(N)=O)n32)[C@@H](CN2CCOC[C@H]2C)CN1]

Tert-butyl (2R,5S)-4-(2-(1-carbamoyl-4-(4-fluorobenzyl)-8,8-dimethyl-7,8-dihydro-6H-pyrrolo[2,3-e][1,2,4]triazolo[4,3-a]pyridin-6-yl)-2-oxoethyl)-2-methyl-5-(((R)-3-methylmorpholino)methyl)piperazine-1-carboxylate (0.084 g, 0.121 mmol, 1.0 eq) was dissolved in TFA (1 mL) and DCM (3 mL) under argon to give a yellow solution. The reaction mixture was stirred at rt for 2 h. Saturated NaHCO₃ (30 mL) was added to the reaction mixture followed by extraction with ethyl acetate (30 mL×2). The combined organic layers were dried over Na₂SO₄, filtered and concentrated. The crude product was purified by column chromatography to give the product (12.8 mg, 17.8%) as a white solid. MS (ESI) m/z 593.8 [M+H]⁺.

Example 95: 2-((2R,5R)-2-((3,3-dimethylmorpholino)methyl)-5-methylpiperazin-1-yl)-1-(4-(4-fluorobenzyl)-8,8-dimethyl-2-(trifluoromethyl)-7,8-dihydro-6H-imidazo[1,2-a]pyrrolo[2,3-e]pyridin-6-yl)ethan-1-one (Compound 146)

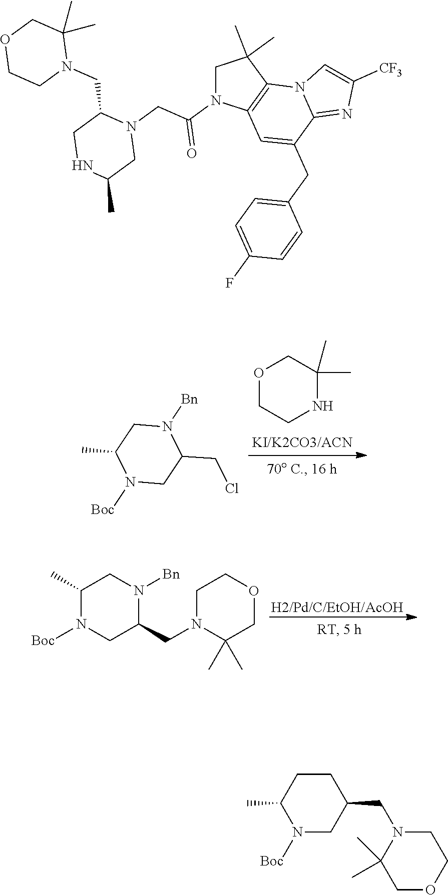

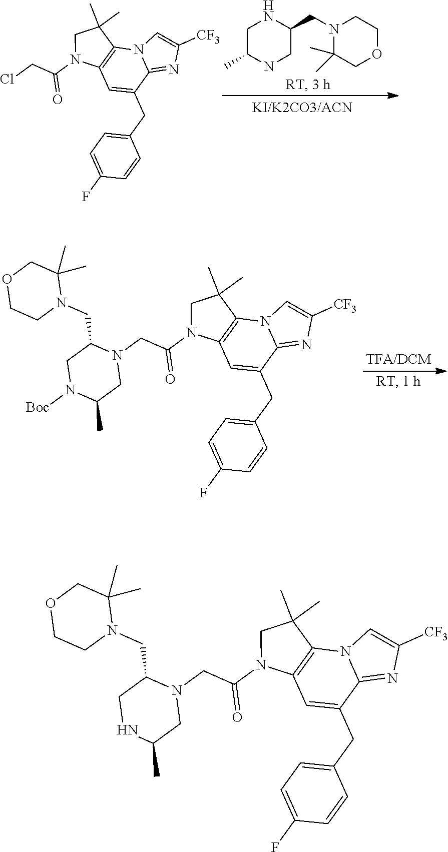

Step 1: tert-butyl (2R,5S)-4-benzyl-5-((3,3-dimethylmorpholino)methyl)-2-methylpiperazine-1-carboxylate

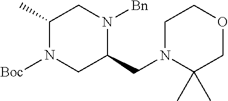 [SMILES: C[C@@H]1CN(Cc2ccccc2)[C@@H](CN2CCOCC2(C)C)CN1C(=O)OC(C)(C)C]

Tert-butyl (2R,5R)-4-benzyl-5-(chloromethyl)-2-methylpiperazine-1-carboxylate (0.4 g, 1.180 mmol, 1.0 eq), 3,3-dimethylmorpholine (0.204 g, 1.771 mmol, 1.5 eq), potassium carbonate (0.489 g, 3.54 mmol, 3.0 eq), and KI (0.392 g, 2.361 mmol, 2.0 eq) were dissolved in acetonitrile (10 mL) under argon to give a colorless suspension. The reaction mixture was stirred at 70° C. for 16 h. Saturated brine (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 20% to give the product (0.48 g, 97%) as a colorless oil. MS (ESI) m/z 418.4 [M+H]⁺.

Step 2: tert-butyl (2R,5S)-5-((3,3-dimethylmorpholino)methyl)-2-methylpiperazine-1-carboxylate

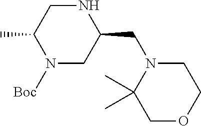 [SMILES: C[C@@H]1CN[C@@H](CN2CCOCC2(C)C)CN1C(=O)OC(C)(C)C]

Above obtained product (0.48 g, 1.149 mmol, 1.0 eq) and Pd/C (0.122 g, 0.575 mmol, 0.5 eq) were dissolved in EtOH (10 mL) and acetic Acid (0.200 mL) under H₂ to give a black suspension. The reaction mixture was stirred at rt for 6 h. The reaction mixture was concentrated to give the product (0.4 g, 100%) as a colorless oil. MS (ESI) m/z 328.4 [M+H]⁺.

Steps 3-4: Following the synthesis of compound 116, compound 146 was obtained as a white solid (18 mg). ¹H NMR (400 MHz, DMSO-d⁶) δ (ppm) 8.68 (s, 1H), 8.17 (s, 1H), 7.38 (dd, J=8.4, 5.7 Hz, 2H), 7.12 (t, J=8.9 Hz, 2H), 4.34-4.22 (m, 2H), 4.12 (d, J=10.6 Hz, 1H), 3.99 (d, J=10.6 Hz, 1H), 3.76 (d, J=16.2 Hz, 1H), 3.51-3.40 (m, 3H), 3.12 (dd, J=25.3, 10.7 Hz, 2H), 2.95 (d, J=10.8 Hz, 1H), 2.67 (d, J=8.8 Hz, 3H), 2.58-2.51 (m, 1H), 2.48-2.43 (m, 1H), 2.39-2.32 (m, 1H), 2.32-2.17 (m, 2H), 2.09-2.00 (m, 1H), 1.55 (d, J=10.2 Hz, 6H), 0.92-0.78 (m, 9H). MS (ESI) m/z 631.6 [M+H]⁺.

Example 96: 2-((2R,5R)-2-((7-oxa-4-azaspiro[2.5]octan-4-yl)methyl)-5-methylpiperazin-1-yl)-1-(4-(4-fluorobenzyl)-8,8-dimethyl-2-(trifluoromethyl)-7,8-dihydro-6H-imidazo[1,2-a]pyrrolo[2,3-e]pyridin-6-yl)ethan-1-one (compound 147)

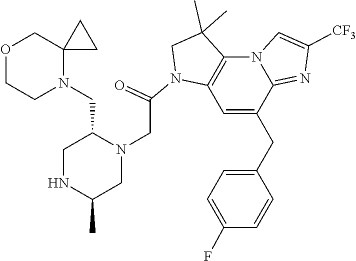 [SMILES: C[C@@H]1CN(CC(=O)N2CC(C)(C)c3c2cc(Cc2ccc(F)cc2)c2nc(C(F)(F)F)cn32)[C@@H](CN2CCOCC23CC3)CN1]

Following the synthesis of compound 146, compound 147 was obtained as a white solid (16 mg). ¹H NMR (400 MHz, DMSO-d⁶) δ (ppm) 8.69 (s, 1H), 8.19 (s, 1H), 7.38 (dd, J=8.4, 5.7 Hz, 2H), 7.12 (t, J=8.9 Hz, 2H), 4.34-4.23 (m, 2H), 4.10 (d, J=10.6 Hz, 1H), 3.94 (d, J=10.6 Hz, 1H), 3.69 (d, J=16.2 Hz, 1H), 3.54-3.43 (m, 2H), 3.29-3.24 (m, 2H), 3.14 (d, J=11.1 Hz, 1H), 2.85-2.77 (m, 1H), 2.76-2.67 (m, 2H), 2.62 (dt, J=9.5, 7.7 Hz, 4H), 2.30-2.07 (m, 3H), 1.54 (d, J=14.2 Hz, 6H), 0.87 (d, J=6.1 Hz, 3H), 0.64-0.51 (m, 2H), 0.40-0.27 (m, 2H). MS (ESI) m/z 629.4 [M+H]⁺.

Example 97: 1-(4-(4-fluorobenzyl)-8,8-dimethyl-7,8-dihydro-6H-pyrazolo[1,5-a]pyrrolo[2,3-e]pyridin-6-yl)-2-((2R,5R)-5-methyl-2-(((R)-3-methylmorpholino)Methyl)piperazin-1-yl)ethan-1-one (compound 148)

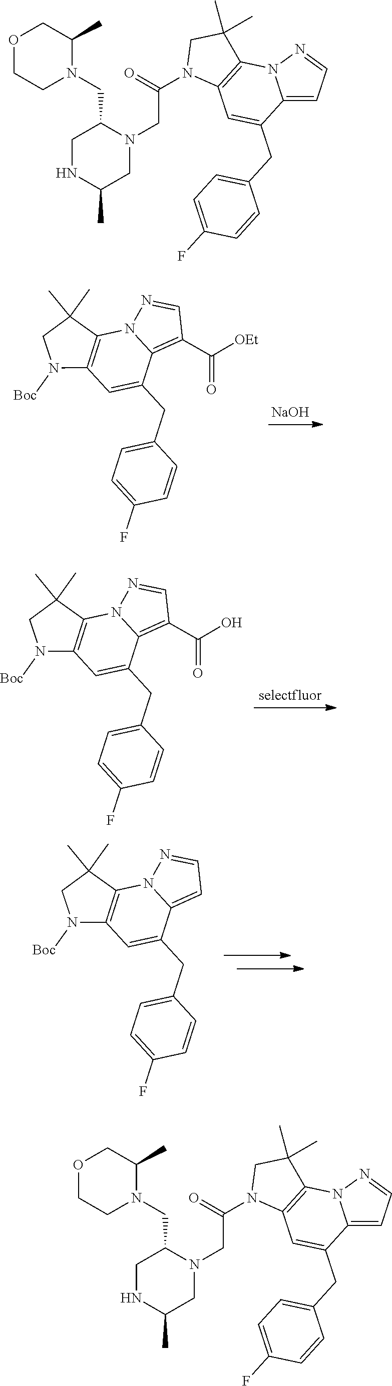

Step 1: 6-(tert-butoxycarbonyl)-4-(4-fluorobenzyl)-8,8-dimethyl-7,8-dihydro-6H-pyrazolo[1,5-a]pyrrolo[2,3-e]pyridine-3-carboxylic acid

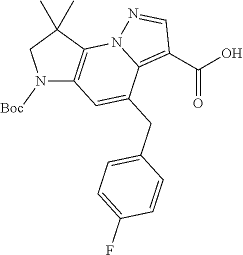 [SMILES: CC(C)(C)OC(=O)N1CC(C)(C)c2c1cc(Cc1ccc(F)cc1)c1c(C(=O)O)cnn21]

At room temperature, NaOH powder (0.07 mL, 0.524 mmol, 4.0 eq) was added into the solution of 6-(tert-butyl) 3-ethyl 4-(4-fluorobenzyl)-8,8-dimethyl-7,8-dihydro-6H-pyrazolo[1,5-a]pyrrolo[2,3-e]pyridine-3,6-dicarboxylate (60 mg, 0.131 mol, 1.0 eq) in methanol and tetrahydrofuran (8 M/L). Then the mixture was heated to reflux for 5 h. TLC showed reaction was complete. 1 M HCl was added to adjust pH to 3-4. The mixture was extracted with ethyl acetate for 3 times. After being dried over sodium sulfate and concentrated, the residue was directly used in next step. LCMS: [M−H]⁺=438.2.

Step 2: tert-butyl 4-(4-fluorobenzyl)-8,8-dimethyl-7,8-dihydro-6H-pyrazolo[1,5-a]pyrrolo[2,3-e]pyridine-6-carboxylate

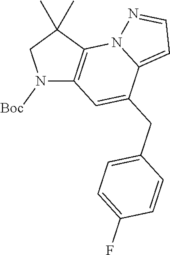 [SMILES: CC(C)(C)OC(=O)N1CC(C)(C)c2c1cc(Cc1ccc(F)cc1)c1ccnn21]

To a solution of above intermediate (30 mg) in DCE/H₂O (0.5 mL/0.5 mL) was added KF (16 mg, 4 eq) and selectfluor (CAS: 140681-55-6, 48 mg, 2 eq) at 80° C. After string for overnight, water added to the residue, and the mixture was extracted with DCM. The solvent was removed in vacuum. The residue was purified by silica gel flash column chromatography using ethyl acetate/hexane (0 to 20%) to afford the title compound. LCMS: [M+H]⁺=396.2.

Following Steps: following the synthesis of compound 14, compound 148 was obtained as a white solid (4.5 mg). MS (ESI) m/z 549.5[M+H]⁺.

Example 98: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrazolo[1,5-a]pyrrolo[2,3-e]pyridine-3-carboxamide (compound 149)

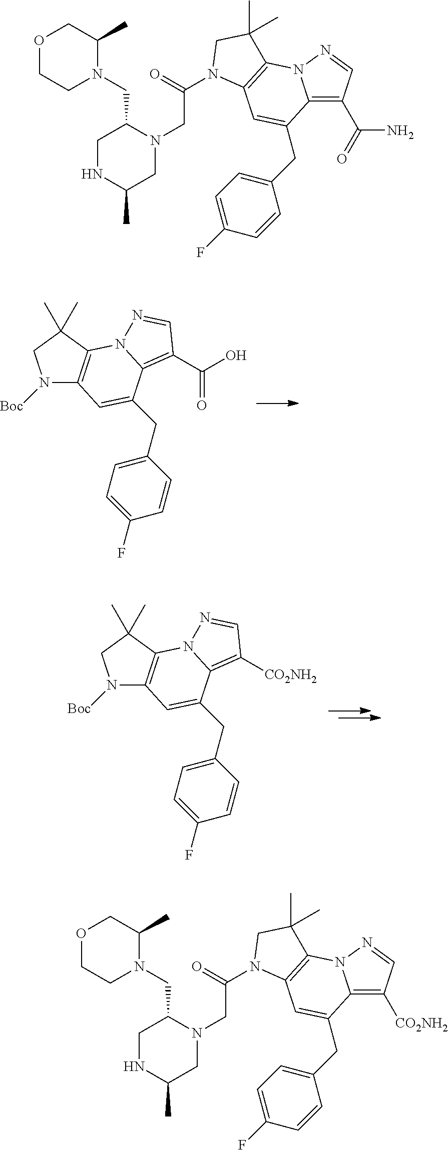

Step 1: tert-butyl 3-((aminooxy)carbonyl)-4-(4-fluorobenzyl)-8,8-dimethyl-7,8-dihydro-6H-pyrazolo[1,5-a]pyrrolo[2,3-e]pyridine-6-carboxylate

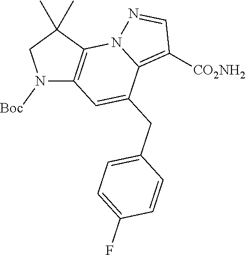

To a suspension of 6-(tert-butoxycarbonyl)-4-(4-fluorobenzyl)-8,8-dimethyl-7,8-dihydro-6H-pyrazolo[1,5-a]pyrrolo[2,3-e]pyridine-3-carboxylic acid (120 mg, 0.27 mmol, 1.0 eq) in toluene (10 mL) was added thionyl chloride (200 mg, 1.64 mmol, 6.0 eq) and the resulting solution was heated to reflux for 2 h. The solvent was removed under reduced pressure, then tetrahydrofuran (10 mL) and ammonia (0.4 mL) were added the resulting solution was stirred for 18 h The solvent was concentrated in vacuum and purified by silica gel flash column chromatography to afford the title compound (50 mg, 25% for two steps).

Following steps: following the synthesis of compound 14, compound 149 was obtained as a white solid (12.5 mg). MS (ESI) m/z 592.5[M+H]⁺. ¹H NMR (400 MHz, DMSO-d⁶) δ (ppm) 8.35 (s, 1H), 8.17 (s, 1H), 7.75 (s, 1H), 7.26-7.15 (m, 3H), 7.13-6.99 (m, 2H), 4.70 (d, J=14.8 Hz, 1H), 4.59 (d, J=14.8 Hz, 1H), 4.12 (d, J=10.6 Hz, 1H), 3.93 (d, J=10.5 Hz, 1H), 3.84-3.64 (m, 2H), 3.52-3.41 (m, 2H), 3.23-3.11 (m, 1H), 3.10-3.00 (m, 1H), 2.97-2.73 (m, 4H), 2.71-2.62 (m, 2H), 2.62-2.54 (m, 1H), 2.39-2.26 (m, 1H), 2.23-2.10 (m, 1H), 2.00-1.87 (m, 1H), 1.79 (d, J=13.0 Hz, 1H), 1.62 (d, J=16.8 Hz, 6H), 0.92 (d, J=4.0 Hz, 6H).

Example 99: 1-(4-(4-fluorobenzyl)-8,8-dimethyl-2-(trifluoromethyl)-7,8-dihydro-6H-pyrazolo[1,5-a]pyrrolo[2,3-e]pyridin-6-yl)-2-((2R,5R)-5-methyl-2-(((R)-3-methylmorpholino)methyl)piperazin-1-yl)ethan-1-one (compound 150)

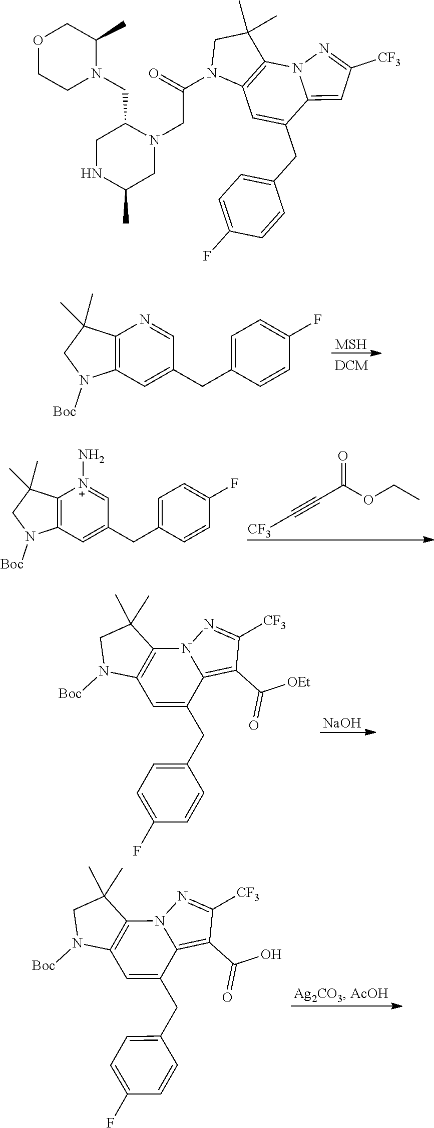

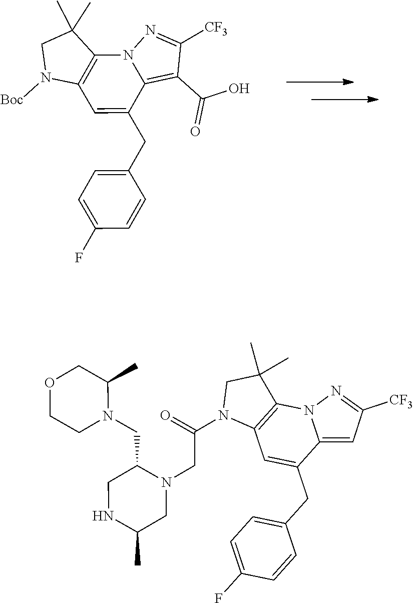 [SMILES: CC(C)(C)OC(=O)N1CC(C)(C)c2c1cc(Cc1ccc(F)cc1)c1c(C(=O)O)c(C(F)(F)F)nn21 ; C[C@@H]1CN(CC(=O)N2CC(C)(C)c3c2cc(Cc2ccc(F)cc2)c2cc(C(F)(F)F)nn32)[C@@H](CN2CCOC[C@H]2C)CN1]

Step 1-2: 6-(tert-butyl) 3-ethyl 4-(4-fluorobenzyl)-8,8-dimethyl-2-(trifluoromethyl)-7,8-dihydro-6H-pyrazolo[1,5-a]pyrrolo[2,3-e]pyridine-3,6-dicarboxylate

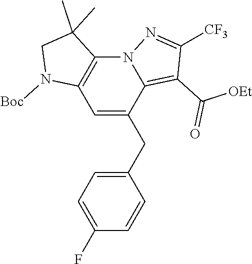 [SMILES: CCOC(=O)c1c(C(F)(F)F)nn2c3c(cc(Cc4ccc(F)cc4)c12)N(C(=O)OC(C)(C)C)CC3(C)C]

To a solution of tert-butyl 6-(4-fluorobenzyl)-3,3-dimethyl-2,3-dihydro-1H-pyrrolo[3,2-b]pyridine-1-carboxylate (1.14 g, 3.2 mmol, 1.0 eq) in DCM (15 mL) was added the reagent MSH (2.41 g, 11.2 mmol, 3.5 eq) at 0° C. and the resulting solution was stirred at room temperature for 18 h. TLC showed reaction was complete. The reaction solution was evaporated. The crude residue was dissolved in DMF (30 mL) at room temperature, ethyl 4,4,4-trifluorobut-2-ynoate (1.06 g, 6.4 mmol, 2.0 eq), K₂CO₃ (1.33 g, 9.6 mmol, 3.0 eq) were added sequentially and the resulting solution was stirred for 4 h. TLC showed reaction was complete. The mixture was diluted with 100 mL of H₂O and extracted with ethyl acetate and dried over sodium sulfate. After concentration, the residue was purified by silica gel flash column chromatography to afford the title compound (0.2 g, 12% for two steps). LCMS: [M+H]⁺=536.1.

Step 3: 6-(tert-butoxycarbonyl)-4-(4-fluorobenzyl)-8,8-dimethyl-2-(trifluoromethyl)-7,8-dihydro-6H-pyrazolo[1,5-a]pyrrolo[2,3-e]pyridine-3-carboxylic acid

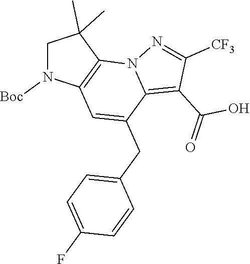 [SMILES: CC(C)(C)OC(=O)N1CC(C)(C)c2c1cc(Cc1ccc(F)cc1)c1c(C(=O)O)c(C(F)(F)F)nn21]

At room temperature, NaOH powder (0.07 mL, 0.524 mmol, 4.0 eq) was added into the solution of above intermediate (60 mg, 0.131 mol, 1.0 eq) in methanol and tetrahydrofuran (8M/L). The mixture was heated to reflux for 5 h. TLC showed reaction was complete, 1M HCl was added to adjust PH to 3-4, and the mixture was extracted with ethyl acetate for 3 times. The organic layer was dried over sodium sulfate and concentrated, the residue was directly used for next step. LCMS: [M−H]⁺=506.2.

Step 4: tert-butyl 4-(4-fluorobenzyl)-8,8-dimethyl-2-(trifluoromethyl)-7,8-dihydro-6H-pyrazolo[1,5-a]pyrrolo[2,3-e]pyridine-6-carboxylate

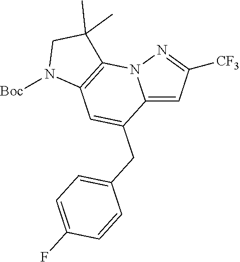 [SMILES: CC(C)(C)OC(=O)N1CC(C)(C)c2c1cc(Cc1ccc(F)cc1)c1cc(C(F)(F)F)nn21]

To a solution of above intermediate (43 mg, crude) in DMSO (1 mL) was added Ag₂CO₃ (6 mg, 27.6 mol %) and acidic acid (1 μL, 16.0 mol %) at 120° C. After stirring for 6 h, water added to the residue, and the mixture was extracted with DCM. The solvent was removed in vacuum to give a crude product, which is directly used in the next step. LCMS: [M+H]⁺=464.2.

Following Steps: following the synthesis of compound 14, compound 150 was obtained as a white solid (3.2 mg). MS (ESI) m/z 617.5[M+H]⁺.

Example 100: 1-((8,8-dimethyl-6-(2-((2R,5R)-5-methyl-2-(((R)-3-methyl morpholino) methyl)piperazin-1-yl)acetyl)-7,8-dihydro-6H-pyrrolo[2,3-e][1,2,4]triazolo[1,5-a]pyridin-4-yl)methyl)-4-fluoropyridin-2(1H)-one (compound 151)

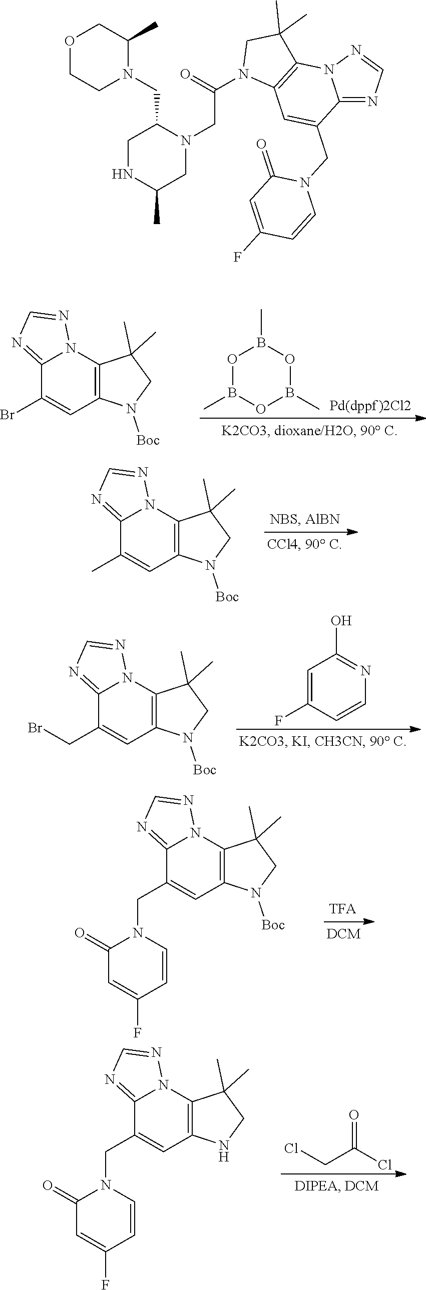

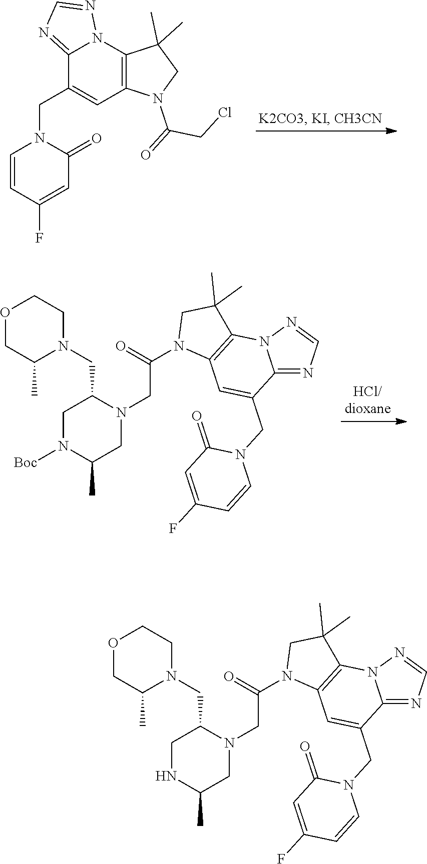 [SMILES: CC1(C)CN(C(=O)CCl)c2cc(Cn3ccc(F)cc3=O)c3ncnn3c21 ; C[C@@H]1COCCN1C[C@H]1CN(C(=O)OC(C)(C)C)[C@H](C)CN1CC(=O)N1CC(C)(C)c2c1cc(Cn1ccc(F)cc1=O)c1ncnn21 ; C[C@@H]1CN(CC(=O)N2CC(C)(C)c3c2cc(Cn2ccc(F)cc2=O)c2ncnn32)[C@@H](CN2CCOC[C@H]2C)CN1]

Step 1: tert-butyl 4,8,8-trimethyl-7,8-dihydro-6H-pyrrolo[2,3-e][1,2,4]triazolo[1,5-a]pyridine-6-carboxylate

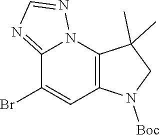 [SMILES: CC(C)(C)OC(=O)N1CC(C)(C)c2c1cc(Br)c1ncnn21]

To a solution of tert-butyl 4-bromo-8,8-dimethyl-7,8-dihydro-6H-pyrrolo[2,3-e][1,2,4]triazolo[1,5-a]pyridine-6-carboxylate (904 mg, 2.462 mmol, 1.0 eq) in dioxane (15 mL)/Water (1.5 mL) were added 2,4,6-trimethyl-1,3,5,2,4,6-trioxatriborinane (464 mg, 3.69 mmol, 1.5 eq), Pd(pdd⁶)₂Cl₂ (180 mg, 0.246 mmol, 0.1 eq), and K₂CO₃ (1021 mg, 7.38 mmol, 3.0 eq) at 25° C. The mixture was stirred at 100° C. for 16 h under N₂. To the reaction mixture were added water and ethyl acetate. The combined organic layer was washed with brine (100 mL), dried over Na₂SO₄, filtered and concentrated. The residue was purified by silica gel flash column chromatography eluting with PE/EA=3/1 to give the title compound (675 mg, 91%) as a yellow solid. MS (ESI) m/z 303.8[M+H]⁺.

Step 2: tert-butyl 4-(bromomethyl)-8,8-dimethyl-7,8-dihydro-6H-pyrrolo[2,3-e][1,2,4]triazol[1,5-a]pyridine-6-carboxylate

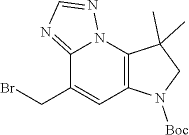 [SMILES: CC(C)(C)OC(=O)N1CC(C)(C)c2c1cc(CBr)c1ncnn21]

To a solution of tert-butyl 4,8,8-trimethyl-7,8-dihydro-6H-pyrrolo[2,3-e][1,2,4]triazolo[1,5-a]pyridine-6-carboxylate (675 mg, 2.232 mmol, 1.0 eq) in CCl₄ (25 mL) were added 1-bromopyrrolidine-2,5-dione (397 mg, 2.232 mmol, 1.0 eq), and (E)-2,2′-(diazene-1,2-diyl) bis (2-methylpropanenitrile) (92 mg, 0.558 mmol, 0.25 eq) at 25° C. The mixture was stirred at 90° C. for 2 h under N₂. The reaction mixture was concentrated. The residue was purified by silica gel flash column chromatography eluting with PE/EA=3/1 to give the title compound (570 mg, 67.0%) as a yellow solid. MS (ESI) m/z 382.9[M+H]+.

Step 3: tert-butyl 4-((4-fluoro-2-oxopyridin-1(2H)-yl)methyl)-8,8-dimethyl-7,8-dihydro-6H-pyrrolo[2,3-e][1,2,4]triazolo[1,5-a]pyridine-6-carboxylate

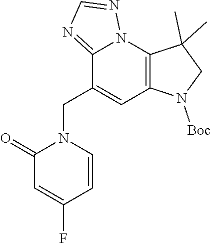 [SMILES: CC(C)(C)OC(=O)N1CC(C)(C)c2c1cc(Cn1ccc(F)cc1=O)c1ncnn21]

To a solution of tert-butyl 4-(bromomethyl)-8,8-dimethyl-7,8-dihydro-6H-pyrrolo[2,3-e][1,2,4]triazolo[1,5-a]pyridine-6-carboxylate (110 mg, 0.289 mmol, 1.0 eq) in acetonitrile (10 mL) were added 4-fluoropyridin-2-ol (48.9 mg, 0.433 mmol, 1.5 eq), K₂CO₃ (120 mg, 0.866 mmol, 3.0 eq) and potassium iodide (71.8 mg, 0.433 mmol, 1.5 eq) at 25° C. and stirred at 90° C. for 2 h under N₂. To the reaction mixture were added water and ethyl acetate. The combined organic layer was washed with brine (100 mL), dried over Na₂SO₄, filtered and concentrated. The residue was purified by silica gel flash column chromatography eluting with PE/EA=3/1 to give the title compound (70 mg, 58.7%) as a yellow solid. MS (ESI) m/z 414.8[M+H]⁺.

Steps 4-7: Following the synthesis of compound 14, compound 151 was obtained as a white solid (57.8 mg). MS (ESI) m/z 567.5[M+H]+.

Example 101: 4-(4-fluorobenzyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 152)

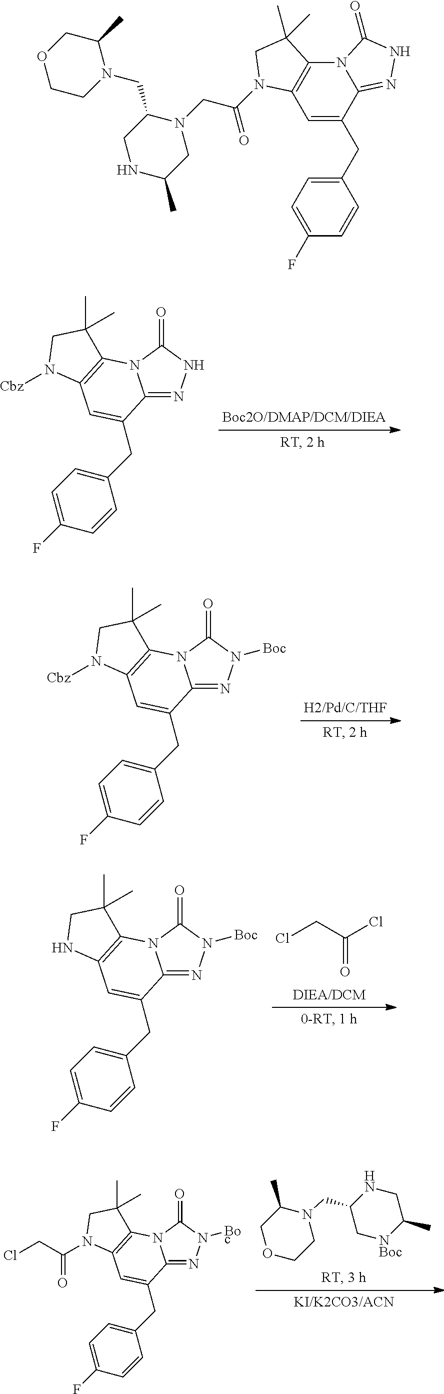

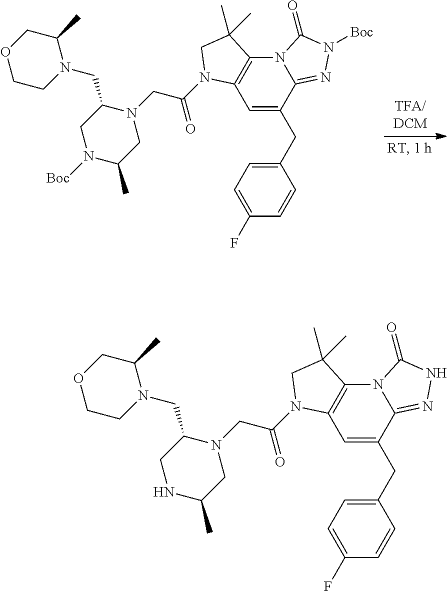 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nn(C(=O)OC(C)(C)C)c(=O)n21 ; C[C@@H]1CN(CC(=O)N2CC(C)(C)c3c2cc(Cc2ccc(F)cc2)c2n[nH]c(=O)n32)[C@@H](CN2CCOC[C@H]2C)CN1]

Step 1: 6-benzyl 2-(tert-butyl) 4-(4-fluorobenzyl)-8,8-dimethyl-1-oxo-7,8-dihydro-1H-pyrrolo[2,3-e][1,2,4]triazolo[4,3-a]pyridine-2,6-dicarboxylate

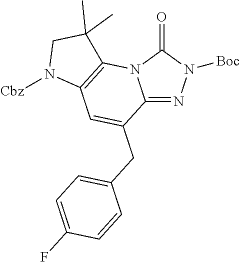 [SMILES: CC(C)(C)OC(=O)n1nc2c(Cc3ccc(F)cc3)cc3c(n2c1=O)C(C)(C)CN3C(=O)OCc1ccccc1]

Benzyl 4-(4-fluorobenzyl)-8,8-dimethyl-1-oxo-1,2,7,8-tetrahydro-6H-pyrrolo[2,3-e][1,2,4]triazolo[4,3-a]pyridine-6-carboxylate (0.08 g, 0.179 mmol, 1.0 eq), Boc₂O (0.059 g, 0.269 mmol, 1.5 eq), DMAP (0.022 g, 0.179 mmol, 1.0 eq), and DIEA (0.046 g, 0.358 mmol, 2.0 eq) were dissolved in DCM (10 mL) under argon to give a yellow solution. The reaction mixture was stirred at rt for 3 h. Saturated NaCl (20 mL) was added to the reaction mixture followed by extraction with dichloromethane (30 mL×2). The combined organic layers were dried over Na₂SO₄, filtered and concentrated. The crude product was added to a silica gel column and was eluted with ethyl acetate/hexane from 0% to 30% to give the product (0.078 g, 80%) as a yellow oil. MS (ESI) m/z 547.5 [M+H]⁺.

Following Steps: following the synthesis of compound 135, compound 152 was obtained as a white solid (31 mg). MS (ESI) m/z 566.4 [M+H]⁺.

Example 102: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[4,3-a]pyridine-1-carbonitrile (compound 153)

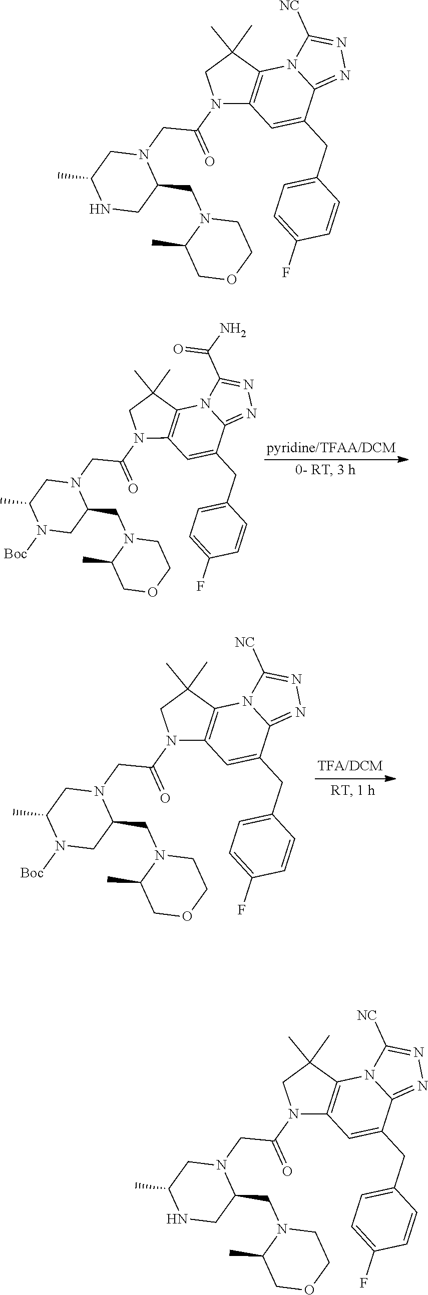 [SMILES: C[C@@H]1CN(CC(=O)N2CC(C)(C)c3c2cc(Cc2ccc(F)cc2)c2nnc(C#N)n32)[C@@H](CN2CCOC[C@H]2C)CN1 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nnc(C(N)=O)n21 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nnc(C#N)n21 ; C[C@@H]1CN(CC(=O)N2CC(C)(C)c3c2cc(Cc2ccc(F)cc2)c2nnc(C#N)n32)[C@@H](CN2CCOC[C@H]2C)CN1]

Step 1: tert-butyl (2R,5S)-4-(2-(1-cyano-4-(4-fluorobenzyl)-8,8-dimethyl-7,8-dihydro-6H-pyrrolo[2,3-e][1,2,4]triazolo[4,3-a]pyridin-6-yl)-2-oxoethyl)-2-methyl-5-(((R)-3-methylmorpholino)methyl)piperazine-1-carboxylate

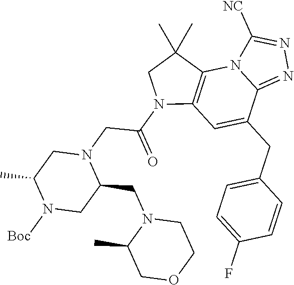 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nnc(C#N)n21]

Tert-butyl (2R,5S)-4-(2-(1-carbamoyl-4-(4-fluorobenzyl)-8,8-dimethyl-7,8-dihydro-6H-pyrrolo[2,3-e][1,2,4]triazolo[4,3-a]pyridin-6-yl)-2-oxoethyl)-2-methyl-5-(((R)-3-methylmorpholino)methyl)piperazine-1-carboxylate (0.024 g, 0.035 mmol, 1.0 eq), TFAA (0.018 g, 0.087 mmol, 2.5 eq), and pyridine (6.85 mg, 0.087 mmol, 2.5 eq) were dissolved in DCM (5 mL) under argon to give a yellow solution. Saturated NaHCO₃ (20 mL) was added to the reaction mixture followed by extraction with dichloromethane (30 mL×2). The combined organic layers were dried over Na₂SO₄, filtered and concentrated. The crude product was added to a silica gel column and was eluted with methanol/dichloromethane from 0% to 5% to give the product (0.012 g, 51.3%) as a yellow oil. MS (ESI) m/z 675.4 [M+H]⁺.

Step 2: compound 153 was obtained as a white solid (5.4 mg) after acidic deprotection and pre-HPLC purification. MS (ESI) m/z 575.6 [M+H]⁺.

Example 103: 1-(4-(4-fluorobenzyl)-2-(1-hydroxyethyl)-8,8-dimethyl-7,8-dihydro-6H-pyrrolo[2,3-e][1,2,4]triazolo[1,5-a]pyridin-6-yl)-2-((2R,5R)-5-methy 1-2-(((R)-3-methylmorpholino)methyl)piperazin-1-yl)ethan-1-one (compound 154)

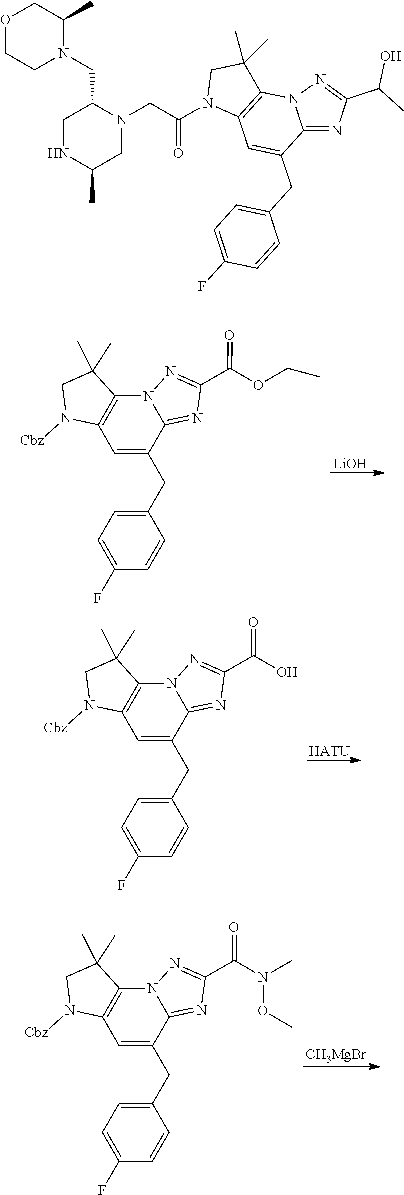

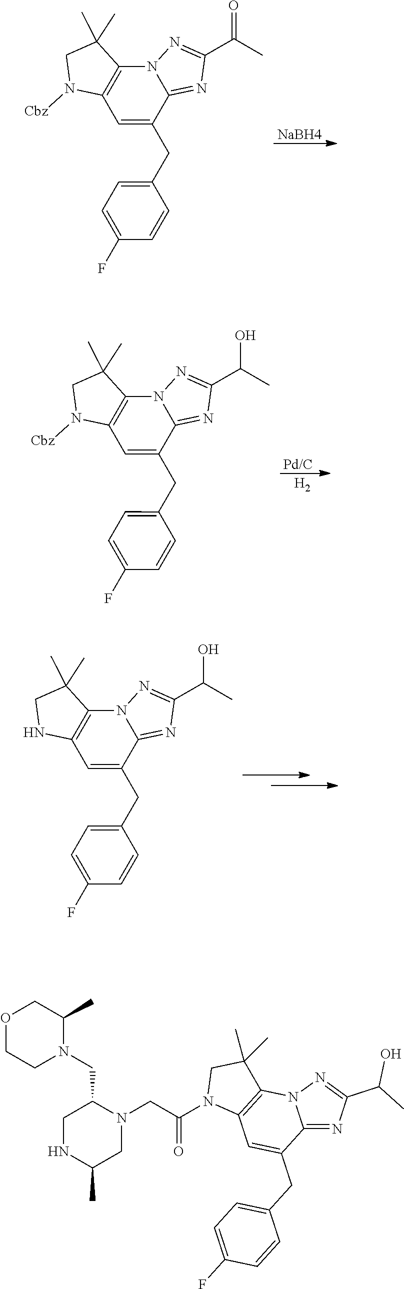

Step 1: 6-((benzyloxy)carbonyl)-4-(4-fluorobenzyl)-8,8-dimethyl-7,8-dihydro-6H-pyrrolo[2,3-e][1,2,4]triazolo[1,5-a]pyridine-2-carboxylic acid

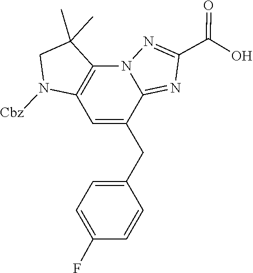 [SMILES: CC1(C)CN(C(=O)OCc2ccccc2)c2cc(Cc3ccc(F)cc3)c3nc(C(=O)O)nn3c21]

In a flame-dried 50 mL round-bottomed flask, 6-benzyl 2-ethyl 4-(4-fluorobenzyl)-8,8-dimethyl-7,8-dihydro-6H-pyrrolo[2,3-e][1,2,4]triazolo[1,5-a]pyridine-2,6-dicarboxylate (400 mg, 0.796 mmol, 1.0 eq) and LiOH (76 mg, 3.18 mmol, 4.0 eq) were dissolved in THF (4 mL) and H₂O (4 mL). The result mixture was stirred for 4 h and adjusted to pH 7 with 1N HCL. H₂O (5 mL) was added to the reaction mixture followed by extraction with ethyl acetate (5 ml×3). The combined organic layers were dried over Na₂SO₄, filtered and concentrated to give the title compound (360 mg, 95%) as a colorless oil. MS (ESI) m/z 475.4 [M+H]⁺.

Step 2: benzyl 4-(4-fluorobenzyl)-2-(methoxy(methyl)carbamoyl)-8,8-dimethyl-7,8-dihydro-6H-pyrrolo[2,3-e][1,2,4]triazolo[1,5-a]pyridine-6-carboxylate

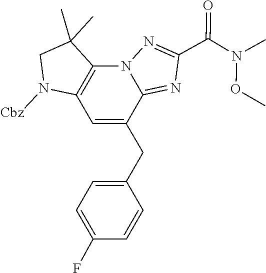 [SMILES: CON(C)C(=O)c1nc2c(Cc3ccc(F)cc3)cc3c(n2n1)C(C)(C)CN3C(=O)OCc1ccccc1]

In a flame-dried 50 mL round-bottomed flask, 6-((benzyloxy)carbonyl)-4-(4-fluorobenzyl)-8,8-dimethyl-7,8-dihydro-6H-pyrrolo[2,3-e][1,2,4]triazolo[1,5-a]pyridine-2-carboxylic acid (360 mg, 0.759 mmol, 1.0 eq), DIPEA (294 mg, 2.276 mmol, 3.0 eq), N,O-dimethylhydroxylamine hydrochloride (89 mg, 0.910 mmol, 1.2 eq) and HATU (456 mg, 0.910 mmol, 1.2 eq) were dissolved in DCM (5 mL). The reaction was stirred for 14 h. H2O (5 ml) was added to the reaction mixture followed by extraction with dichloromethane (5 mL×2). The combined organic layers were dried over Na₂SO₄, filtered and concentrated. The crude product was added to a silica gel column and was eluted with EA/PE from 0˜40% to give the title compound (320 mg, 81.4%) as an off-white solid.

MS (ESI) m/z 518.9 [M+H]⁺.

Step 3: benzyl 2-acetyl-4-(4-fluorobenzyl)-8,8-dimethyl-7,8-dihydro-6H-pyrrolo[2,3-e][1,2,4]triazolo[1,5-a]pyridine-6-carboxylate

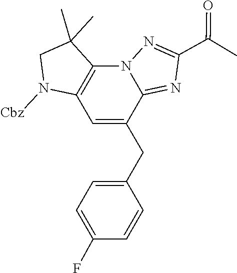 [SMILES: CC(=O)c1nc2c(Cc3ccc(F)cc3)cc3c(n2n1)C(C)(C)CN3C(=O)OCc1ccccc1]

In a flame-dried 50 mL round-bottomed flask, benzyl 4-(4-fluorobenzyl)-2-(methoxy(methyl)carbamoyl)-8,8-dimethyl-7,8-dihydro-6H-pyrrolo[2,3-e][1,2,4]triazolo[1,5-a]pyridine-6-carboxylate (440 mg, 0.850 mmol, 1.0 eq) was dissolved in THE (5 mL). methylmagnesium bromide (405 mg, 3.40 mmol, 4.0 eq) in THF (1.7 mL) was added to the reaction mixture dropwise over 10 min. The reaction was stirred for 2 h. Saturated NH₄Cl (5 ml) was added to the reaction mixture followed by extraction with ethyl acetate (10 ml×3). The combined organic layers were dried over Na₂SO₄, filtered and concentrated. The crude product was added to a silica gel column and was eluted with EA/PE from 0˜40% to give the title compound (280 mg, 69.7%) as a colorless oil. MS (ESI) m/z 473.9 [M+H]⁺.

Step 4: benzyl 4-(4-fluorobenzyl)-2-(1-hydroxyethyl)-8,8-dimethyl-7,8-dihydro-6H-pyrrolo[2,3-e][1,2,4]triazolo[1,5-a]pyridine-6-carboxylate

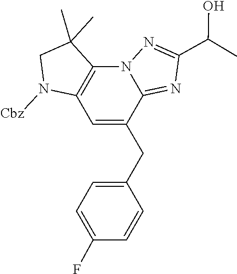 [SMILES: CC(O)c1nc2c(Cc3ccc(F)cc3)cc3c(n2n1)C(C)(C)CN3C(=O)OCc1ccccc1]

In a flame-dried 25 mL round-bottomed flask, benzyl 2-acetyl-4-(4-fluorobenzyl)-8,8-dimethyl-7,8-dihydro-6H-pyrrolo[2,3-e][1,2,4]triazolo[1,5-a]pyridine-6-carboxylate (80 mg, 0.169 mmol, 1.0 eq) was dissolved in MeOH (5 mL). NaBH₄ (25.6 mg, 0.677 mmol, 4.0 eq) was added to the reaction mixture. The reaction was stirred for 2 h. Saturated NH₄Cl (5 ml) was added to the reaction mixture followed by extraction with ethyl acetate (10 ml×3). The combined organic layers were dried over Na₂SO₄, filtered and concentrated. The crude product was added to a silica gel column and was eluted with EA/PE from 0˜40% to give the title compound (60 mg, 74.7%) as a colorless oil. MS (ESI) m/z 473.9 [M+H]⁺.

Step 5: benzyl 4-(4-fluorobenzyl)-2-(1-hydroxyethyl)-8,8-dimethyl-7,8-dihydro-6H-pyrrolo[2,3-e][1,2,4]triazolo[1,5-a]pyridine-6-carboxylate

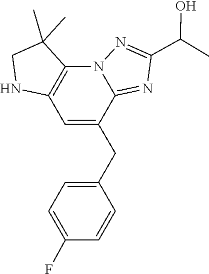 [SMILES: CC(O)c1nc2c(Cc3ccc(F)cc3)cc3c(n2n1)C(C)(C)CN3]

In a flame-dried 25 mL round-bottomed flask, benzyl 2-acetyl-4-(4-fluorobenzyl)-8,8-dimethyl-7,8-dihydro-6H-pyrrolo[2,3-e][1,2,4]triazolo[1,5-a]pyridine-6-carboxylate (80 mg, 0.169 mmol, 1.0 eq) was dissolved in MeOH (5 mL). NaBH₄ (25.6 mg, 0.677 mmol, 4.0 eq) was added to the reaction mixture. The reaction was stirred for 2 h. Saturated NH₄Cl (5 ml) was added to the reaction mixture followed by extraction with ethyl acetate (10 ml×3). The combined organic layers were dried over Na₂SO₄, filtered and concentrated. The crude product was added to a silica gel column and was eluted with PE/EA from 0˜40% to give the title compound (60 mg, 74.7%) as a colorless oil. MS (ESI) m/z 341.7 [M+H]⁺.

Following Steps: compound 154 was obtained (22 mg, 42.84%) as a white solid in a manner similar to that described in compound 14. MS (ESI) m/z 594.6 [M+H]⁺.

Example 104: 1-(4-(2,4-difluorobenzyl)-8,8-dimethyl-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (compound 155)

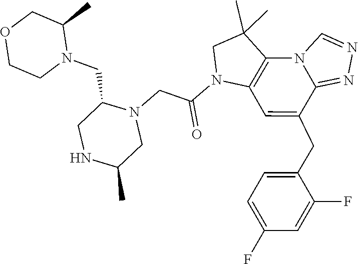 [SMILES: C[C@@H]1CN(CC(=O)N2CC(C)(C)c3c2cc(Cc2ccc(F)cc2F)c2nncn32)[C@@H](CN2CCOC[C@H]2C)CN1]

Compound 155 was obtained as a white solid (24 mg) in a manner similar to that described in compound 9. ¹H NMR (400 MHz, DMSO-d⁶) δ (ppm) 9.64 (s, 1H), 8.08 (s, 1H), 7.55 (dd, J=15.5, 8.5 Hz, 1H), 7.29 (td, J=10.1, 2.5 Hz, 1H), 7.10 (t, J=8.3 Hz, 1H), 4.46-4.26 (m, 2H), 4.20-4.03 (m, 1H), 3.95-3.87 (m, 1H), 3.85-3.61 (m, 2H), 3.55-3.45 (m, 2H), 3.30-2.55 (m, 10H), 2.39-2.28 (m, 1H), 2.24-2.13 (m, 1H), 2.02-1.89 (m, 1H), 1.87-1.74 (m, 1H), 1.57 (d, J=17.4 Hz, 6H), 1.11-0.84 (m, 6H). MS (ESI) m/z 568.53 [M+H]⁺

Example 105: 1-(4-(2,4-difluorobenzyl)-1,8,8-trimethyl-7,8-dihydro-6H-pyrrolo[2,3-e][1,2,4]triazolo[4,3-a]pyridin-6-yl)-2-((2R,5R)-5-methyl-2-(((R)-3-m ethylmorpholino)methyl)piperazin-1-yl)ethan-1-one (compound 156)

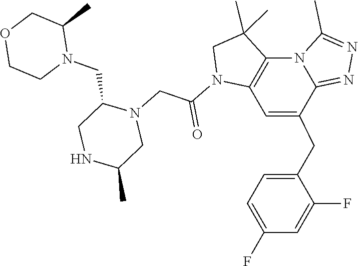 [SMILES: Cc1nnc2c(Cc3ccc(F)cc3F)cc3c(n12)C(C)(C)CN3C(=O)CN1C[C@@H](C)NC[C@@H]1CN1CCOC[C@H]1C]

Compound 156 was obtained as a white solid (2.5 mg) in a manner similar to that described in compound 10. MS (ESI) m/z 582.54 [M+H]⁺

Example 106: 1-(1-(difluoromethyl)-4-(4-fluorobenzyl)-8,8-dimethyl-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (compound 157)

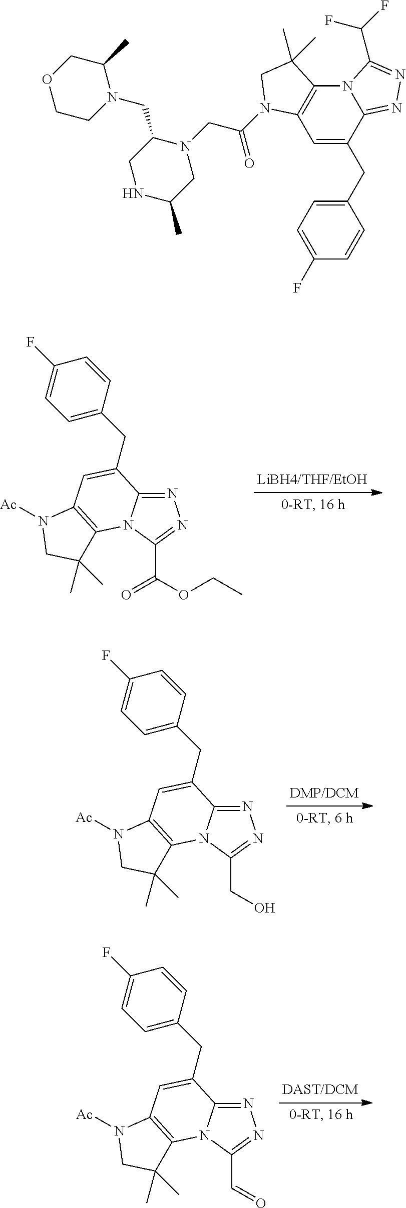 [SMILES: C[C@@H]1CN(CC(=O)N2CC(C)(C)c3c2cc(Cc2ccc(F)cc2)c2nnc(C(F)F)n32)[C@@H](CN2CCOC[C@H]2C)CN1 ; CCOC(=O)c1nnc2c(Cc3ccc(F)cc3)cc3c(n12)C(C)(C)CN3C(C)=O ; CC(=O)N1CC(C)(C)c2c1cc(Cc1ccc(F)cc1)c1nnc(CO)n21 ; CC(=O)N1CC(C)(C)c2c1cc(Cc1ccc(F)cc1)c1nnc(C=O)n21]

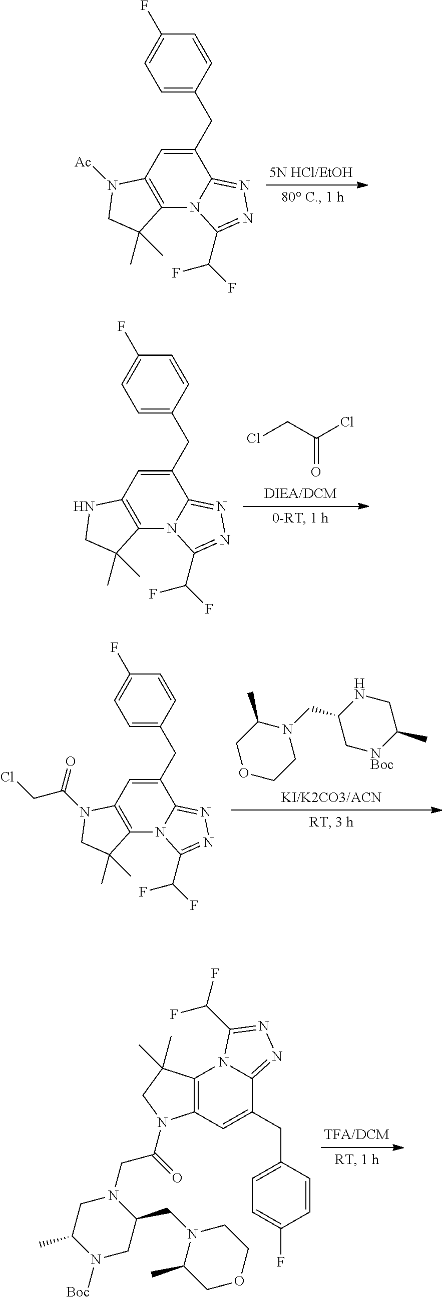 [SMILES: CC(=O)N1CC(C)(C)c2c1cc(Cc1ccc(F)cc1)c1nnc(C(F)F)n21 ; CC1(C)CNc2cc(Cc3ccc(F)cc3)c3nnc(C(F)F)n3c21 ; O=C(Cl)CCl ; CC1(C)CN(C(=O)CCl)c2cc(Cc3ccc(F)cc3)c3nnc(C(F)F)n3c21 ; C[C@@H]1COCCN1C[C@H]1CN(C(=O)OC(C)(C)C)[C@H](C)CN1 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nnc(C(F)F)n21]

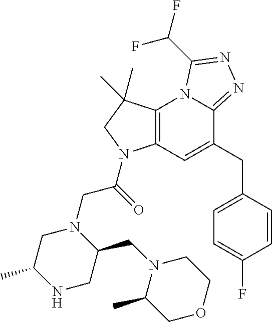 [SMILES: C[C@@H]1CN(CC(=O)N2CC(C)(C)c3c2cc(Cc2ccc(F)cc2)c2nnc(C(F)F)n32)[C@@H](CN2CCOC[C@H]2C)CN1]

Step 1: 1-(4-(4-fluorobenzyl)-1-(hydroxymethyl)-8,8-dimethyl-7,8-dihydro-6H-pyrrolo[2,3-e][1,2,4]triazolo[4,3-a]pyridin-6-yl)ethan-1-one

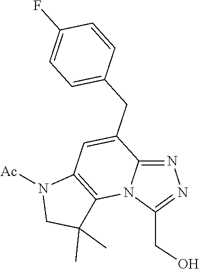 [SMILES: CC(=O)N1CC(C)(C)c2c1cc(Cc1ccc(F)cc1)c1nnc(CO)n21]

Ethyl 6-acetyl-4-(4-fluorobenzyl)-8,8-dimethyl-7,8-dihydro-6H-pyrrolo[2,3-e][1,2,4]triazolo[4,3-a]pyridine-1-carboxylate (0.378 g, 0.921 mmol, 1.0 eq) was dissolved in EtOH (2 mL) and THF (10.00 mL) under argon to give a yellow solution. The reaction mixture was stirred at rt for 16 h. Saturated NH₄Cl (20 mL) was added to the reaction mixture followed by extraction with ethyl acetate (30 mL×2). The combined organic layers were dried over Na₂SO₄, filtered and concentrated. The crude product was added to a silica gel column and was eluted with methanol/dichloromethane from 0% to 6% to give the product (0.1 g, 29.5%) as a yellow solid. MS (ESI) m/z 369.4 [M+H]⁺.

Step 2: 6-acetyl-4-(4-fluorobenzyl)-8,8-dimethyl-7,8-dihydro-6H-pyrrolo[2,3-e][1,2,4]triazolo[4,3-a]pyridine-1-carbaldehyde

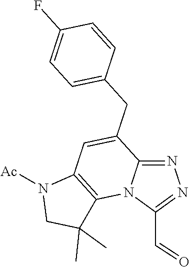 [SMILES: CC(=O)N1CC(C)(C)c2c1cc(Cc1ccc(F)cc1)c1nnc(C=O)n21]

1-(4-(4-fluorobenzyl)-1-(hydroxymethyl)-8,8-dimethyl-7,8-dihydro-6H-pyrrolo[2,3-e][1,2,4]triazolo[4,3-a]pyridin-6-yl)ethan-1-one (0.1 g, 0.271 mmol, 1.0 eq) and DMP (0.230 g, 0.543 mmol, 2.0 eq) were dissolved in DCM (15 mL) under argon to give a yellow solution. The reaction mixture was stirred at rt for 5 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 80% to give the product (0.076 g, 76%) as a yellow solid. MS (ESI) m/z 367.6 [M+H]⁺.

Step 3: 1-(1-(difluoromethyl)-4-(4-fluorobenzyl)-8,8-dimethyl-7,8-dihydro-6H-pyrrolo[2,3-e][1,2,4]triazolo[4,3-a]pyridin-6-yl)ethan-1-one

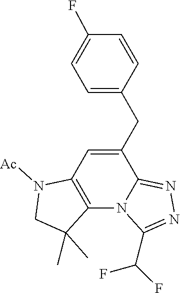 [SMILES: CC(=O)N1CC(C)(C)c2c1cc(Cc1ccc(F)cc1)c1nnc(C(F)F)n21]

6-acetyl-4-(4-fluorobenzyl)-8,8-dimethyl-7,8-dihydro-6H-pyrrolo[2,3-e][1,2,4]triazolo[4,3-a]pyridine-1-carbaldehyde (0.076 g, 0.207 mmol, 1.0 eq) and DAST (0.100 g, 0.622 mmol, 3.0 eq) were dissolved in DCM (10 mL) under argon to give a yellow solution. The reaction mixture was stirred at rt for 16 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 30% to give the product (0.02 g, 24.83%) as a yellow oil. MS (ESI) m/z 389.4 [M+H]⁺.

Following Steps: following the synthesis of compound 14, compound 157 was obtained as a white solid (3.4 mg). ¹H NMR (400 MHz, DMSO-d⁶) δ (ppm) 8.33 (s, 1H), 7.47-7.34 (m, 3H), 7.16-7.09 (m, 2H), 4.43-4.35 (m, 2H), 4.06 (d, J=10.4 Hz, 1H), 3.86 (d, J=10.3 Hz, 1H), 3.79 (s, 2H), 3.47-3.44 (m, 2H), 3.17-3.11 (m, 1H), 2.97-2.83 (m, 4H), 2.77-2.72 (m, 2H), 2.69-2.64 (m, 1H), 2.35-2.31 (m, 1H), 2.21-2.12 (m, 1H), 1.98-1.95 (m, 2H), 1.83 (d, J=11.3 Hz, 1H), 1.54-1.48 (m, 6H), 1.01-0.89 (m, 6H). MS (ESI) m/z 600.3 [M+H]⁺.

Example 107: 1-(4-((2,4-dimethylthiazol-5-yl)methyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methyl morpholino)methyl)piperazin-1-yl)ethan-1-one (compound 158)

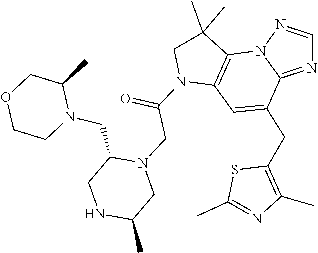 [SMILES: Cc1nc(C)c(Cc2cc3c(n4ncnc24)C(C)(C)CN3C(=O)CN2C[C@@H](C)NC[C@@H]2CN2CCOC[C@H]2C)s1]

Compound 158 was obtained as a white solid (44.9 mg) in a manner similar to that described in compound 132. MS (ESI) m/z 567.5[M+H]⁺. ¹H NMR (400 MHz, DMSO-d⁶) δ (ppm) 8.51 (s, 1H), 8.38 (s, 1H), 4.38 (s, 2H), 4.14 (d, J=10.6 Hz, 1H), 3.95 (d, J=10.6 Hz, 1H), 3.80 (d, J=16.7 Hz, 1H), 3.66 (d, J=16.6 Hz, 1H), 3.50-3.43 (m, 2H), 3.18-3.08 (m, 1H), 3.05-2.95 (m, 1H), 2.94-2.77 (m, 3H), 2.76-2.69 (m, 1H), 2.68-2.57 (m, 2H), 2.50 (s, 3H), 2.37-2.24 (m, 4H), 2.22-2.09 (m, 1H), 1.99-1.86 (m, 1H), 1.76 (d, J=11.6 Hz, 1H), 1.58 (d, J=16.8 Hz, 6H), 0.96-0.83 (m, 6H).

Example 108: 1-(4-(4-fluorobenzyl)-2-(3-hydroxyazetidin-1-yl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 159)

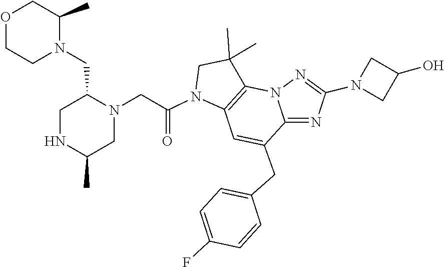 [SMILES: C[C@@H]1CN(CC(=O)N2CC(C)(C)c3c2cc(Cc2ccc(F)cc2)c2nc(N4CC(O)C4)nn32)[C@@H](CN2CCOC[C@H]2C)CN1]

Following the synthesis of compound 195, compound 159 was obtained as a white solid. MS (ESI) m/z 621.6 [M+H]⁺.

Example 109: 1-(4-(4-fluorobenzyl)-8,8-dimethyl-2-(trifluoromethyl)-7,8-dihydro-6H-imidazo[1,2-a]pyrrolo[2,3-e]pyridin-6-yl)-2-((2R,5R)-2-((3-(hydroxy methyl)morpholino)methyl)-5-methylpiperazin-1-yl)ethan-1-one (compound 160)

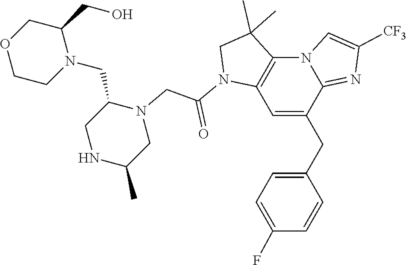 [SMILES: C[C@@H]1CN(CC(=O)N2CC(C)(C)c3c2cc(Cc2ccc(F)cc2)c2nc(C(F)(F)F)cn32)[C@@H](CN2CCOC[C@H]2CO)CN1]

Following the synthesis of compound 146, compound 160 was obtained as a white solid (39 mg). ¹H NMR (400 MHz, DMSO-d⁶) δ (ppm) 8.68 (s, 1H), 8.20 (s, 1H), 7.38 (dd, J=8.0, 5.8 Hz, 2H), 7.12 (t, J=8.7 Hz, 2H), 4.65 (s, 1H), 4.35-4.19 (m, 2H), 4.08-3.97 (m, 2H), 3.84 (d, J=16.8 Hz, 1H), 3.71 (d, J=16.9 Hz, 1H), 3.54 (d, J=9.0 Hz, 1H), 3.48 (s, 2H), 3.41 (d, J=11.2 Hz, 1H), 3.23-3.16 (m, 1H), 3.15-3.04 (m, 2H), 2.94 (dd, J=12.7, 8.5 Hz, 1H), 2.79 (dd, J=27.0, 11.2 Hz, 2H), 2.73-2.59 (m, 2H), 2.56 (d, J=11.1 Hz, 1H), 2.31 (t, J=11.1 Hz, 1H), 2.15-2.04 (m, 1H), 1.98-1.82 (m, 2H), 1.54 (d, J=13.2 Hz, 6H), 0.91 (d, J=6.0 Hz, 3H). MS (ESI) m/z 633.5 [M+H]⁺.

Example 110: 2-((2R,5R)-2-(((3R,5R)-3,5-dimethylmorpholino)methyl)-5-methylpiperazin-1-yl)-1-(4-(4-fluorobenzyl)-8,8-dimethyl-2-(trifluoromethyl)-7,8-dihydro-6H-imidazo[1,2-a]pyrrolo[2,3-e]pyridin-6-yl)ethan-1-one

2-((2R,5R)-2-(((3S,5S)-3,5-dimethylmorpholino)methyl)-5-methylpiperazin-1-yl)-1-(4-(4-fluorobenzyl)-8,8-dimethyl-2-(trifluoromethyl)-7,8-dihydro-6H-imidazo[1,2-a]pyrrolo[2,3-e]pyridin-6-yl)ethan-1-one (Compound 161/162)

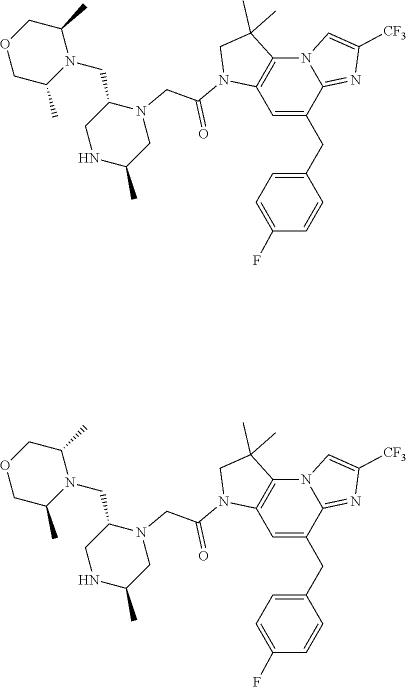 [SMILES: C[C@@H]1CN(CC(=O)N2CC(C)(C)c3c2cc(Cc2ccc(F)cc2)c2nc(C(F)(F)F)cn32)[C@@H](CN2[C@H](C)COC[C@H]2C)CN1 ; C[C@@H]1CN(CC(=O)N2CC(C)(C)c3c2cc(Cc2ccc(F)cc2)c2nc(C(F)(F)F)cn32)[C@@H](CN2[C@@H](C)COC[C@@H]2C)CN1]

Following the synthesis of compound 146, compounds 161 and 162 were obtained as diastereoisomers after pre-HPLC purification.

Compound 161: white solid (18.8 mg). ¹H NMR (400 MHz, DMSO-d⁶) δ (ppm) 8.69 (s, 1H), 8.12 (s, 1H), 7.37 (dd, J=8.2, 5.8 Hz, 2H), 7.13 (t, J=8.8 Hz, 2H), 4.35-4.22 (m, 2H), 4.09 (d, J=10.6 Hz, 1H), 3.97 (d, J=10.6 Hz, 1H), 3.67 (d, J=16.4 Hz, 1H), 3.43 (d, J=8.4 Hz, 2H), 3.37-3.35 (m, 1H), 3.18-3.09 (m, 2H), 3.03 (d, J=10.4 Hz, 1H), 2.84-2.76 (m, 1H), 2.75-2.64 (m, 4H), 2.48-2.42 (m, 1H), 2.38-2.16 (m, 3H), 1.54 (dd, J=13.5, 5.4 Hz, 6H), 0.91 (d, J=6.1 Hz, 3H), 0.85 (t, J=8.4 Hz, 6H). MS (ESI) m/z 631.6 [M+H]⁺.

Compound 162: white solid (9.1 mg). ¹H NMR (400 MHz, DMSO-d⁶) δ (ppm) 8.69 (s, 1H), 8.16 (s, 1H), 7.38 (dd, J=8.4, 5.7 Hz, 2H), 7.12 (t, J=8.9 Hz, 2H), 4.38-4.22 (m, 2H), 4.13 (d, J=10.5 Hz, 1H), 3.93 (d, J=10.6 Hz, 1H), 3.82 (d, J=16.4 Hz, 1H), 3.56 (d, J=16.3 Hz, 1H), 3.38 (d, J=8.8 Hz, 2H), 3.14-3.00 (m, 2H), 2.95-2.79 (m, 3H), 2.79-2.70 (m, 2H), 2.67-2.59 (m, 2H), 2.40 (t, J=11.0 Hz, 1H), 2.36-2.26 (m, 1H), 2.07-1.95 (m, 1H), 1.55 (d, 6H), 0.94-0.81 (m, 9H). MS (ESI) m/z 631.6 [M+H]⁺.

Example 111: 1-(4-(4-fluorobenzyl)-8,8-dimethyl-2-(tetrahydrofuran-3-yl)-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 163)

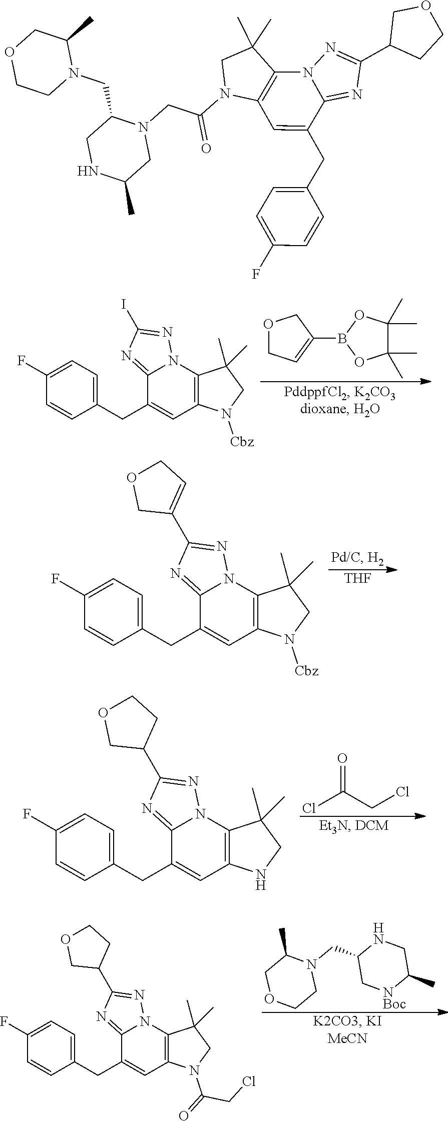

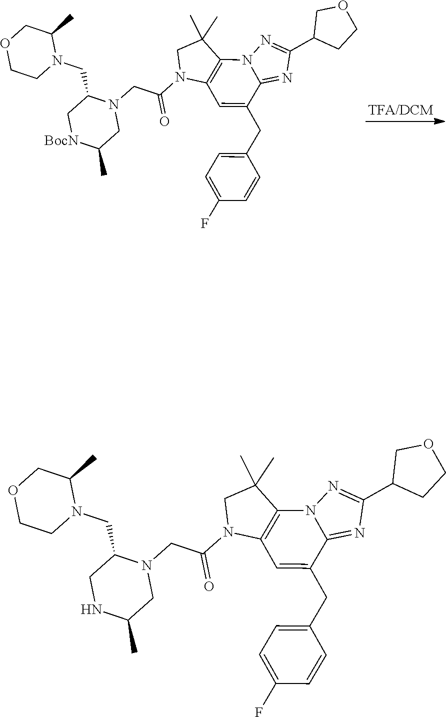 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C3CCOC3)nn21 ; C[C@@H]1CN(CC(=O)N2CC(C)(C)c3c2cc(Cc2ccc(F)cc2)c2nc(C4CCOC4)nn32)[C@@H](CN2CCOC[C@H]2C)CN1]

Step 1: benzyl 2-(2,5-dihydrofuran-3-yl)-4-(4-fluorobenzyl)-8,8-dimethyl-7,8-dihydro-6H-pyrrolo[2,3-e][1,2,4]triazolo[1,5-a]pyridine-6-carboxylate

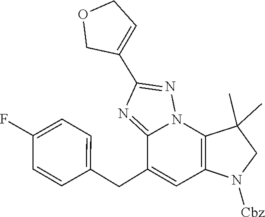 [SMILES: CC1(C)CN(C(=O)OCc2ccccc2)c2cc(Cc3ccc(F)cc3)c3nc(C4=CCOC4)nn3c21]

A mixture of benzyl 4-(4-fluorobenzyl)-2-iodo-8,8-dimethyl-7,8-dihydro-6H-pyrrolo[2,3-e][1,2,4]triazolo[1,5-a]pyridine-6-carboxylate (300 mg, 0.539 mmol, 1.0 eq), 2-(2,5-dihydrofuran-3-yl)-4,4,5,5-tetramethyl-1,3,2-dioxaborolane (211 mg, 1.078 mmol, 2.0 eq), PdCl₂(dppf) (39.5 mg, 0.054 mmol, 0.1 eq) and K₂CO₃ (224 mg, 1.618 mmol, 3.0 eq) in dioxane (20 mL)/water (4 mL) was stirred at 100° C. for 6 h. The mixture was diluted with water, extracted with ethyl acetate (twice). the organic layer was washed with brine, dried over Na₂SO₄ and concentrated to give a yellow oil, which was purified by column eluted with 30% EA/PE to give the title compound (230 mg, 86%) as a white solid. MS (ESI) m/z 499.46 [M+H]⁺

Step 2: 4-(4-fluorobenzyl)-8,8-dimethyl-2-(tetrahydrofuran-3-yl)-7,8-dihydro-6H-pyrrolo[2,3-e][1,2,4]triazolo[1,5-a]pyridine

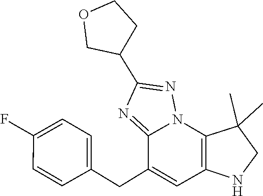 [SMILES: CC1(C)CNc2cc(Cc3ccc(F)cc3)c3nc(C4CCOC4)nn3c21]

A mixture of benzyl 2-(2,5-dihydrofuran-3-yl)-4-(4-fluorobenzyl)-8,8-dimethyl-7,8-dihydro-6H-pyrrolo[2,3-e][1,2,4]triazolo[1,5-a]pyridine-6-carboxylate (230 mg, 0.461 mmol, 1.0 eq) and Pd/C (45 mg, 20%) in THF (20 mL) was degassed and purged with H₂ for 3 times. The reaction mixture was stirred at 20-25° C. for 16 h. the mixture was filtered and the filtrated was concentrated to give the title compound as a yellow oil, which was used in next step directly. MS (ESI) m/z 367.38 [M+H]⁺

Step 3: 2-chloro-1-(4-(4-fluorobenzyl)-8,8-dimethyl-2-(tetrahydrofuran-3-yl)-7,8-dihydro-6H-pyrrolo[2,3-e][1,2,4]triazolo[1,5-a]pyridin-6-yl)ethan-1-one

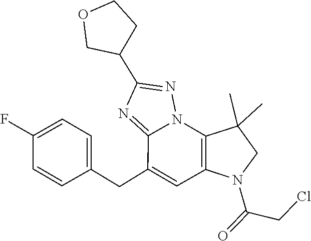 [SMILES: CC1(C)CN(C(=O)CCl)c2cc(Cc3ccc(F)cc3)c3nc(C4CCOC4)nn3c21]

To a mixture of 4-(4-fluorobenzyl)-8,8-dimethyl-2-(tetrahydrofuran-3-yl)-7,8-dihydro-6H-pyrrolo[2,3-e][1,2,4]triazolo[1,5-a]pyridine (160 mg, 0.437 mmol, 1.0 eq) and Et₃N (88 mg, 0.873 mmol, 2.0 eq) in DCM (10 mL) was added 2-chloroacetyl chloride (99 mg, 0.873 mmol, 2.0 eq) at 0-10° C. The reaction mixture was stirred for 1 h. The mixture was quenched with H₂O, extracted with DCM. The organic layer was washed with brine, dried over Na₂SO₄ and concentrated to give the title compound (180 mg, 93%) as a yellow oil. MS (ESI) m/z 443.36 [M+H]⁺

Step 4: tert-butyl (2R,5S)-4-(2-(4-(4-fluorobenzyl)-8,8-dimethyl-2-(tetrahydrofuran-3-yl)-7,8-dihydro-6H-pyrrolo[2,3-e][1,2,4]triazolo[1,5-a]pyridin-6-yl)-2-oxoethyl)-2-methyl-5-(((R)-3-m ethylmorpholino)methyl)piperazine-1-carboxylate

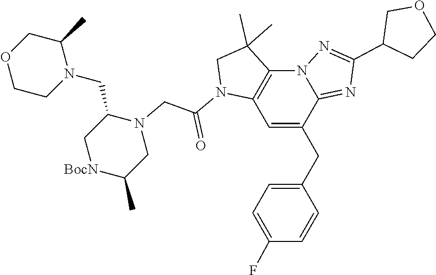 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(C3CCOC3)nn21]

A mixture of 2-chloro-1-(4-(4-fluorobenzyl)-8,8-dimethyl-2-(tetrahydrofuran-3-yl)-7,8-dihydro-6H-pyrrolo[2,3-e][1,2,4]triazolo[1,5-a]pyridin-6-yl)ethan-1-one (180 mg, 0.406 mmol, 1.0 eq), tert-butyl (2R,5S)-2-methyl-5-(((R)-3-methylmorpholino)methyl)piperazine-1-carboxylate (127 mg, 0.406 mmol, 1.0 eq), K₂CO₃ (112 mg, 0.813 mmol, 2.0 eq) and KI (101 mg, 0.610 mmol, 1.5 eq) in acetonitrile (20 mL) was stirred at 20-25° C. for 16 h. The mixture was diluted with water, extracted with ethyl acetate (twice). The organic layer was washed with brine, dried over Na₂SO₄ and concentrated to give a yellow oil, which was purified by column eluted with 3% MeOH/DCM to give the title compound (220 mg, 75%) as a yellow oil. MS (ESI) m/z 720.66 [M+H]⁺

Step 5: 1-(4-(4-fluorobenzyl)-8,8-dimethyl-2-(tetrahydrofuran-3-yl)-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 163)

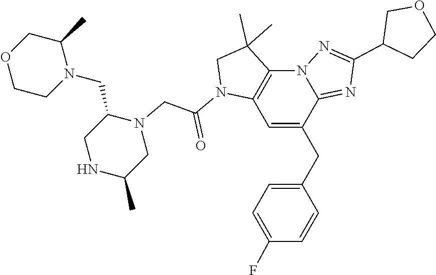 [SMILES: C[C@@H]1CN(CC(=O)N2CC(C)(C)c3c2cc(Cc2ccc(F)cc2)c2nc(C4CCOC4)nn32)[C@@H](CN2CCOC[C@H]2C)CN1]

A mixture of tert-butyl (2R,5S)-4-(2-(4-(4-fluorobenzyl)-8,8-dimethyl-2-(tetrahydrofuran-3-yl)-7,8-dihydro-6H-pyrrolo[2,3-e][1,2,4]triazolo[1,5-a]pyridin-6-yl)-2-oxoethyl)-2-methyl-5-(((R)-3-m ethylmorpholino)methyl)piperazine-1-carboxylate (220 mg, 0.306 mmol, 1.0 eq) in DCM (5 mL)/TFA (5.00 mL) was stirred at 20-25° C. for 2 h. The mixture was concentrated. the residue was dissolved with DCM, washed with NaHCO₃(aq), brine, dried over Na₂SO₄ and concentrated to give a yellow oil, which was purified by prep-HPLC to give the title compound (98 mg, 51.7%) as a white solid. ¹H NMR (400 MHz, DMSO-d⁶) δ (ppm) 8.31 (s, 1H), 7.42 (dd, J=7.8, 5.8 Hz, 2H), 7.15 (t, J=8.6 Hz, 2H), 4.34-4.23 (m, 2H), 4.19-4.10 (m, 2H), 4.01-3.67 (m, 7H), 3.60-3.43 (m, 3H), 3.21-3.04 (m, 2H), 2.97-2.56 (m, 7H), 2.43-2.31 (m, 3H), 2.24-2.12 (m, 1H), 2.02-1.91 (m, 1H), 1.88-1.76 (m, 1H), 1.61 (d, J=17.9 Hz, 6H), 1.03-0.84 (m, 6H). MS (ESI) m/z 620.57 [M+H]⁺

Example 112: 1-(8,8-dimethyl-4-(2,4,6-trifluorobenzyl)-7,8-dihydro-6H-pyrrolo[2,3-e][1,2,4]triazolo[4,3-a]pyridin-6-yl)-2-((2R,5R)-5-methyl-2-(((R)-3-methylmorpholino)methyl)piperazin-1-yl)ethan-1-one (compound 164)

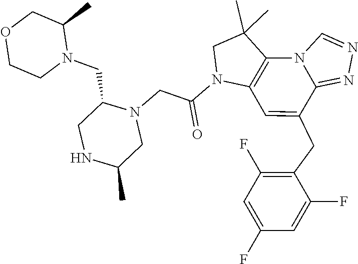 [SMILES: C[C@@H]1CN(CC(=O)N2CC(C)(C)c3c2cc(Cc2c(F)cc(F)cc2F)c2nncn32)[C@@H](CN2CCOC[C@H]2C)CN1]

Compound 164 was obtained as a white solid (18 mg) in a manner similar to that described in compound 9. ¹H NMR (400 MHz, DMSO-d⁶) δ (ppm) 9.64 (s, 1H), 8.02 (s, 1H), 7.30 (t, J=8.5 Hz, 2H), 4.43-4.30 (m, 2H), 4.13 (d, J=10.6 Hz, 1H), 3.91 (d, J=10.6 Hz, 1H), 3.82-3.59 (m, 2H), 3.54-3.45 (m, 2H), 3.22-3.13 (m, 1H), 3.07-2.97 (m, 1H), 2.95-2.59 (m, 7H), 2.51-2.43 (m, 1H), 2.37-2.28 (m, 1H), 2.22-2.12 (m, 1H), 2.00-1.90 (m, 1H), 1.82-1.72 (m, 1H), 1.58 (d, J=18.4 Hz, 6H), 0.97-0.87 (m, 6H). MS (ESI) m/z 568.53 [M+H]⁺

Example 113: 1-(2-cyclopropyl-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 165)

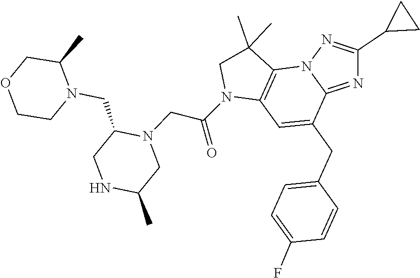 [SMILES: C[C@@H]1CN(CC(=O)N2CC(C)(C)c3c2cc(Cc2ccc(F)cc2)c2nc(C4CC4)nn32)[C@@H](CN2CCOC[C@H]2C)CN1]

Compound 165 was obtained as a white solid in a manner similar to that described in compound 163. MS (ESI) m/z 590.6 [M+H]⁺. ¹H NMR (400 MHz, DMSO-d⁶) δ (ppm) 8.28 (s, 1H), 7.43-7.34 (m, 2H), 7.19-7.08 (m, 2H), 4.23 (d, J=3.8 Hz, 2H), 4.13 (d, J=10.6 Hz, 1H), 3.93 (d, J=10.6 Hz, 1H), 3.81 (d, J=16.7 Hz, 1H), 3.67 (d, J=16.6 Hz, 1H), 3.53-3.45 (m, 2H), 3.15 (t, J=10.3 Hz, 1H), 3.03 (s, 1H), 2.95-2.72 (m, 3H), 2.68-2.61 (m, 2H), 2.52-2.47 (m, 3H), 2.32 (t, J=10.7 Hz, 1H), 2.26-2.12 (m, 2H), 1.94 (t, J=9.4 Hz, 1H), 1.78 (d, J=12.3 Hz, 1H), 1.62 (s, 3H), 1.57 (s, 3H), 1.13-0.99 (m, 4H), 0.92 (dd, J=6.2, 1.4 Hz, 6H).

Example 114: 1-(4-(difluoro(3-(trifluoromethyl)bicyclo[1.1.1]pentan-1-yl) methyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 166)

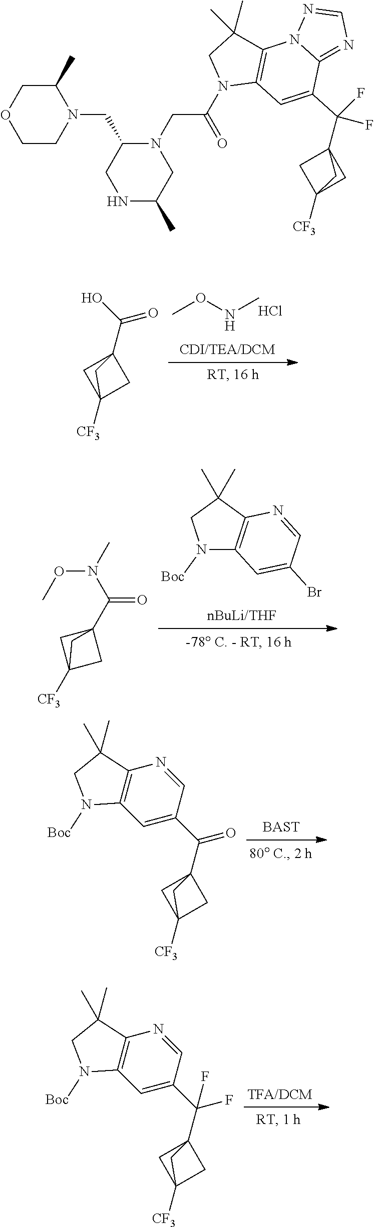

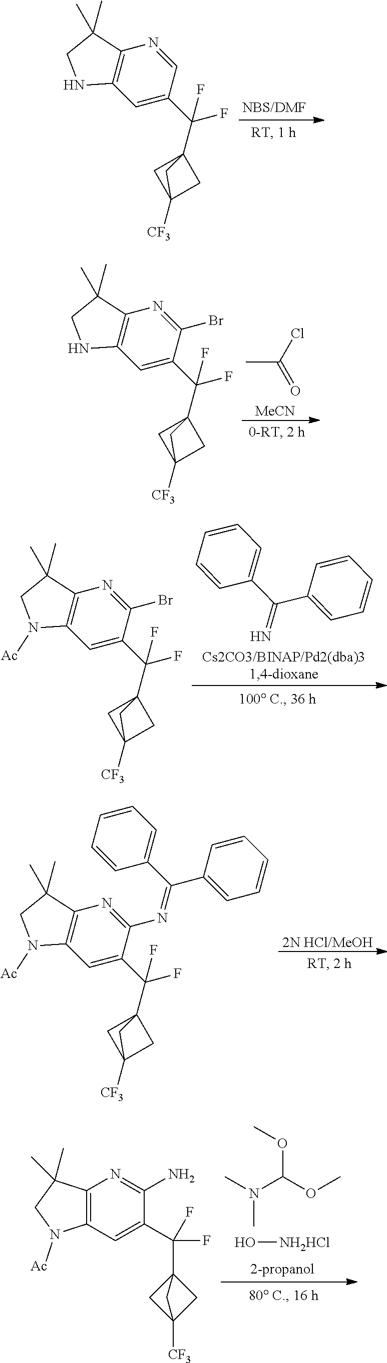

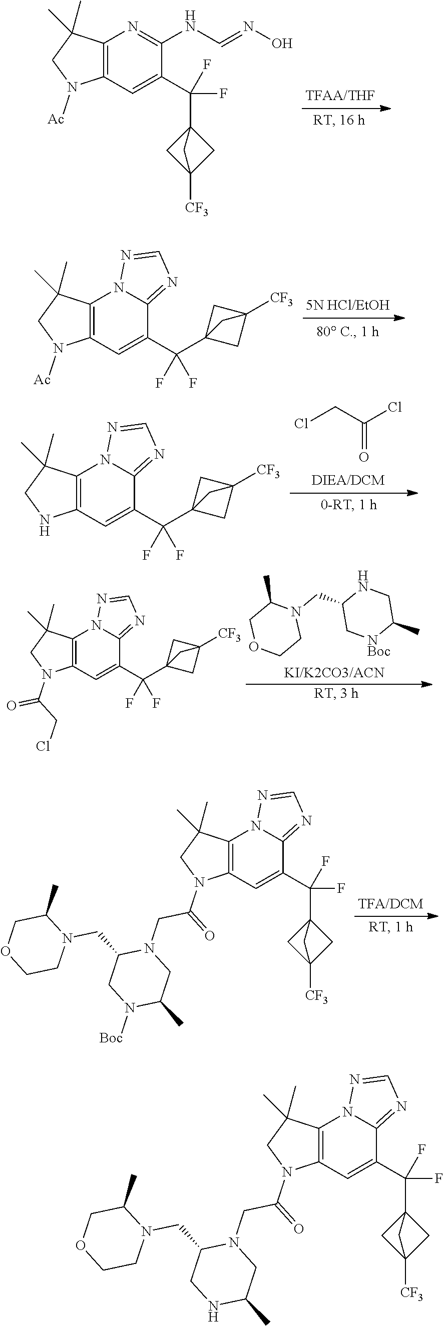

Step 1: N-methoxy-N-methyl-3-(trifluoromethyl)bicyclo[1.1.1]pentane-1-carboxamide

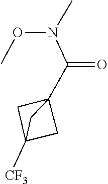

3-(Trifluoromethyl)bicyclo[1.1.1]pentane-1-carboxylic acid (0.6 g, 3.33 mmol, 1.0 eq), N,O-dimethylhydroxylamine (0.244 g, 4.00 mmol, 1.2 eq), TEA (0.674 g, 6.66 mmol, 2.0 eq), and CDI (0.594 g, 3.66 mmol, 1.1 eq) were dissolved in DCM (20 mL) under argon to give a colorless solution. The reaction mixture was stirred at rt for 16 h. Saturated NaCl (20 mL) was added to the reaction mixture followed by extraction with dichloromethane (30 mL×2). The combined organic layers were dried over Na₂SO₄, filtered and concentrated. The crude product was added to a silica gel column and was eluted with ethyl acetate/hexane from 0% to 30% to give the product (0.41 g, 55.1%) as a colorless liquid. MS (ESI) m/z 224.3 [M+H]⁺.

Step 2: tert-butyl 3,3-dimethyl-6-(3-(trifluoromethyl)bicyclo[1.1.1]pentane-1-carbonyl)-2,3-dihydro-1H-pyrrolo[3,2-b]pyridine-1-carboxylate

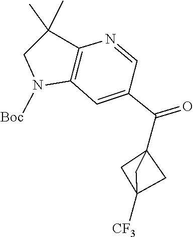 [SMILES: CC(C)(C)OC(=O)N1CC(C)(C)c2ncc(C(=O)C34CC(C(F)(F)F)(C3)C4)cc21]

Tert-butyl 6-bromo-3,3-dimethyl-2,3-dihydro-1H-pyrrolo[3,2-b]pyridine-1-carboxylate (0.6 g, 1.834 mmol, 1.0 eq) was dissolved in THF (15 mL) under argon to give a colorless solution. The reaction mixture was cooled to −78° C. with a dry ice/acetone bath. n-BuLi (0.235 g, 3.67 mmol, 2.0 eq) was added to the reaction mixture dropwise. The reaction mixture was stirred at −78° C. for 30 mins. N-methoxy-N-methyl-3-(trifluoromethyl)bicyclo[1.1.1]pentane-1-carboxamide (0.409 g, 1.834 mmol, 1.0 eq) was added to the reaction mixture dropwise. The reaction mixture was stirred at −78° C. for 1 h. Saturated NH₄Cl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 20% to give the product (0.41 g, 54.5%) as a yellow oil. MS (ESI) m/z 411.2 [M+H]⁺.

Step 3: tert-butyl 6-(difluoro(3-(trifluoromethyl)bicyclo[1.1.1]pentan-1-yl)methyl)-3,3-dimethyl-2,3-di hydro-1H-pyrrolo[3,2-b]pyridine-1-carboxylate

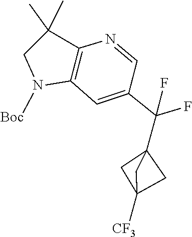 [SMILES: CC(C)(C)OC(=O)N1CC(C)(C)c2ncc(C(F)(F)C34CC(C(F)(F)F)(C3)C4)cc21]

Tert-butyl 3,3-dimethyl-6-(3-(trifluoromethyl)bicyclo[1.1.1]pentane-1-carbonyl)-2,3-dihydro-1H-pyrrolo[3,2-b]pyridine-1-carboxylate (0.41 g, 0.999 mmol, 1.0 eq) was dissolved in BAST (3 mL) under argon to give a yellow solution. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 15% to give the product (0.23 g, 53.2%) as a colorless oil. MS (ESI) m/z 433.4 [M+H]⁺.

Step 4: 6-(difluoro(3-(trifluoromethyl)bicyclo[1.1.1]pentan-1-yl)methyl)-3,3-dimethyl-2,3-di hydro-1H-pyrrolo[3,2-b]pyridine

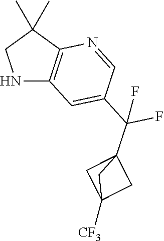 [SMILES: CC1(C)CNc2cc(C(F)(F)C34CC(C(F)(F)F)(C3)C4)cnc21]

Tert-butyl 6-(difluoro(3-(trifluoromethyl)bicyclo[1.1.1]pentan-1-yl)methyl)-3,3-dimethyl-2,3-di hydro-1H-pyrrolo[3,2-b]pyridine-1-carboxylate (0.27 g, 0.624 mmol, 1.0 eq) was dissolved in DCM (6 mL) and TFA (3.00 mL) under argon to give a yellow solution. The reaction mixture was stirred at rt for 2 h. Saturated NaHCO₃ (20 mL) was added to the reaction mixture followed by extraction with ethyl acetate (30 mL×2). The combined organic layers were dried over Na₂SO₄, filtered and concentrated to give the product (0.207 g, 100%) as a yellow oil. MS (ESI) m/z 333.2 [M+H]⁺.

Step 5: 5-bromo-6-(difluoro(3-(trifluoromethyl)bicyclo[1.1.1]pentan-1-yl)methyl)-3,3-dimeth yl-2,3-dihydro-1H-pyrrolo[3,2-b]pyridine

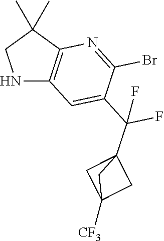 [SMILES: CC1(C)CNc2cc(C(F)(F)C34CC(C(F)(F)F)(C3)C4)c(Br)nc21]

6-(Difluoro(3-(trifluoromethyl)bicyclo[1.1.1]pentan-1-yl)methyl)-3,3-dimethyl-2,3-dihydro-1H-pyrrolo[3,2-b]pyridine (0.207 g, 0.623 mmol, 1.0 eq) and NBS (0.111 g, 0.623 mmol, 1.0 eq) were dissolved in DMF (5 mL) under argon to give a yellow solution. The reaction mixture was stirred at 0° C. for 2 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 30% to give the product (0.23 g, 90%) as a white solid. MS (ESI) m/z 411.2 [M+H]⁺.

Step 6: 1-(5-bromo-6-(difluoro(3-(trifluoromethyl)bicyclo[1.1.1]pentan-1-yl)methyl)-3,3-dim ethyl-2,3-dihydro-1H-pyrrolo[3,2-b]pyridin-1-yl)ethan-1-one

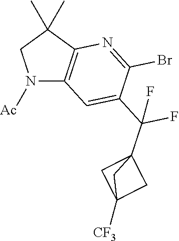 [SMILES: CC(=O)N1CC(C)(C)c2nc(Br)c(C(F)(F)C34CC(C(F)(F)F)(C3)C4)cc21]

5-Bromo-6-(difluoro(3-(trifluoromethyl)bicyclo[1.1.1]pentan-1-yl)methyl)-3,3-dim ethyl-2,3-dihydro-1H-pyrrolo[3,2-b]pyridine (0.23 g, 0.559 mmol, 1.0 eq) and AcCl (0.066 g, 0.839 mmol, 1.5 eq) were dissolved in Acetonitrile (10 mL) under argon to give a yellow solution. The reaction mixture was stirred at rt for 2 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 50% to give the product (0.23 g, 91%) as a white solid. MS (ESI) m/z 453.2 [M+H]⁺.

Step 7: 1-(6-(difluoro(3-(trifluoromethyl)bicyclo[1.1.1]pentan-1-yl)methyl)-5-((diphenylmethylene)amino)-3,3-dimethyl-2,3-dihydro-1H-pyrrolo[3,2-b]pyridin-1-yl)ethan-1-one

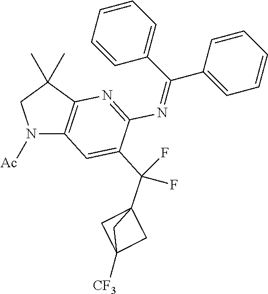 [SMILES: CC(=O)N1CC(C)(C)c2nc(N=C(c3ccccc3)c3ccccc3)c(C(F)(F)C34CC(C(F)(F)F)(C3)C4)cc21]

1-(5-bromo-6-(difluoro(3-(trifluoromethyl)bicyclo[1.1.1]pentan-1-yl)methyl)-3,3-dimethyl-2,3-dihydro-1H-pyrrolo[3,2-b]pyridin-1-yl)ethan-1-one (0.23 g, 0.507 mmol, 1.0 eq), diphenylmethanimine (0.184 g, 1.015 mmol, 2 eq), Cs₂CO₃ (0.331 g, 1.015 mmol, 2.0 eq), BINAP (0.063 g, 0.101 mmol, 0.2 eq), and Pd₂(dba)₃ (0.046 g, 0.051 mmol, 0.1 eq) were dissolved in 1,4-dioxane (15 mL) under argon to give a yellow suspension. The reaction mixture was stirred at 100° C. for 36 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 40% to give the product (0.28 g, 100%) as a yellow oil. MS (ESI) m/z 554.5 [M+H]⁺.

Step 8: 1-(5-amino-6-(difluoro(3-(trifluoromethyl)bicyclo[1.1.1]pentan-1-yl)methyl)-3,3-dim ethyl-2,3-dihydro-1H-pyrrolo[3,2-b]pyridin-1-yl)ethan-1-one

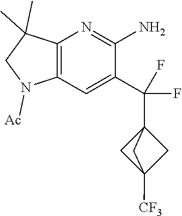 [SMILES: CC(=O)N1CC(C)(C)c2nc(N)c(C(F)(F)C34CC(C(F)(F)F)(C3)C4)cc21]

1-(6-(difluoro(3-(trifluoromethyl)bicyclo[1.1.1]pentan-1-yl)methyl)-5-((diphenylmethylene)amino)-3,3-dimethyl-2,3-dihydro-1H-pyrrolo[3,2-b]pyridin-1-yl)ethan-1-one (0.28 g, 0.506 mmol, 1.0 eq) and HCl (2 N, 3 mL) were dissolved in MeOH (6 mL) under argon to give a yellow solution. The reaction mixture was stirred at rt for 2 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 50% to give the product (0.114 g, 57.9%) as a yellow solid. MS (ESI) m/z 390.3 [M+H]⁺.

Step 9: (E)-N-(1-acetyl-6-(difluoro(3-(trifluoromethyl)bicyclo[1.1.1]pentan-1-yl)methyl)-3,3-dimethyl-2,3-dihydro-1H-pyrrolo[3,2-b]pyridin-5-yl)-N′-hydroxyformimidamide

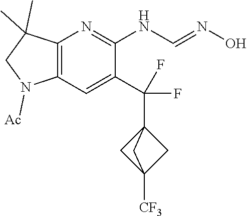 [SMILES: CC(=O)N1CC(C)(C)c2nc(N/C=N/O)c(C(F)(F)C34CC(C(F)(F)F)(C3)C4)cc21]

1-(5-amino-6-(difluoro(3-(trifluoromethyl)bicyclo[1.1.1]pentan-1-yl)methyl)-3,3-di methyl-2,3-dihydro-1H-pyrrolo[3,2-b]pyridin-1-yl)ethan-1-one (0.114 g, 0.293 mmol, 1.0 eq) and N,N-dimethylformamide dimethyl acetal (0.105 g, 0.878 mmol, 3.0 eq) were dissolved in 2-propanol (10 mL) under argon to give a yellow solution. The reaction mixture was stirred at 80° C. for 16 h. Hydroxylamine hydrochloride (0.061 g, 0.878 mmol, 3.0 eq) was added to the reaction mixture in one portion. The reaction mixture was stirred at 50° C. for 2 h. The reaction mixture was concentrated to give the product (0.127 g, 100%). MS (ESI) m/z 433.6 [M+H]⁺.

Step 10: 1-(4-(difluoro(3-(trifluoromethyl)bicyclo[1.1.1]pentan-1-yl)methyl)-8,8-dimethyl-7,8-dihydro-6H-pyrrolo[2,3-e][1,2,4]triazolo[1,5-a]pyridin-6-yl)ethan-1-one

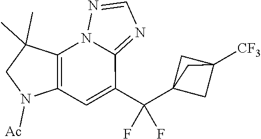 [SMILES: CC(=O)N1CC(C)(C)c2c1cc(C(F)(F)C13CC(C(F)(F)F)(C1)C3)c1ncnn21]

(E)-N-(1-acetyl-6-(difluoro(3-(trifluoromethyl)bicyclo[1.1.1]pentan-1-yl)methyl)-3,3-dimethyl-2,3-dihydro-1H-pyrrolo[3,2-b]pyridin-5-yl)-N′-hydroxyformimidamide (0.127 g, 0.294 mmol, 1.0 eq) and TFAA (0.185 g, 0.881 mmol, 3.0 eq) were dissolved in THF (10 mL) under argon to give a yellow suspension. The reaction mixture was stirred at rt for 2 h. Saturated NaHCO₃ (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 50% to give the product (0.097 g, 80%) as a white solid. MS (ESI) m/z 415.3 [M+H]⁺.

Following Steps: following the synthesis of compound 14, compound 166 was obtained as a white solid (49.4 mg). MS (ESI) m/z 626.7 [M+H]⁺.

Example 115: 1-(4-(4-fluorobenzyl)-8,8-dimethyl-2-(trifluoromethyl)-7,8-dihydro-6H-imidazo[1,2-a]pyrrolo[2,3-e]pyridin-6-yl)-2-((2R,5R)-2-((3-(fluoromethyl)morpholino)methyl)-5-methylpiperazin-1-yl)ethan-1-one (compound 167)

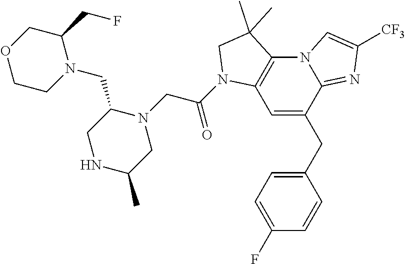 [SMILES: C[C@@H]1CN(CC(=O)N2CC(C)(C)c3c2cc(Cc2ccc(F)cc2)c2nc(C(F)(F)F)cn32)[C@@H](CN2CCOC[C@H]2CF)CN1]

Following the synthesis of compound 146, compound 167 was obtained as a white solid (23 mg). ¹H NMR (400 MHz, DMSO-d⁶) δ (ppm) 8.68 (s, 1H), 8.20 (s, 1H), 7.43-7.33 (m, 2H), 7.18-7.02 (m, 2H), 4.76-4.53 (m, 1H), 4.36-4.22 (m, 2H), 4.14-3.99 (m, 1H), 3.91 (t, J=10.0 Hz, 1H), 3.79-3.68 (m, 1H), 3.68-3.47 (m, 3H), 3.24-3.14 (m, 1H), 3.07-2.95 (m, 1H), 2.96-2.81 (m, 2H), 2.79-2.68 (m, 1H), 2.68-2.54 (m, 4H), 2.47-2.31 (m, 2H), 2.31-2.16 (m, 2H), 2.10-1.92 (m, 1H), 1.54 (dd, J=6.2, 2.9 Hz, 6H), 0.88 (d, J=6.0 Hz, 3H). MS (ESI) m/z 635.4 [M+H]⁺.

Example 116: 1-(4-(6-fluoro-3,4-dihydroquinolin-1(2H)-yl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 168)

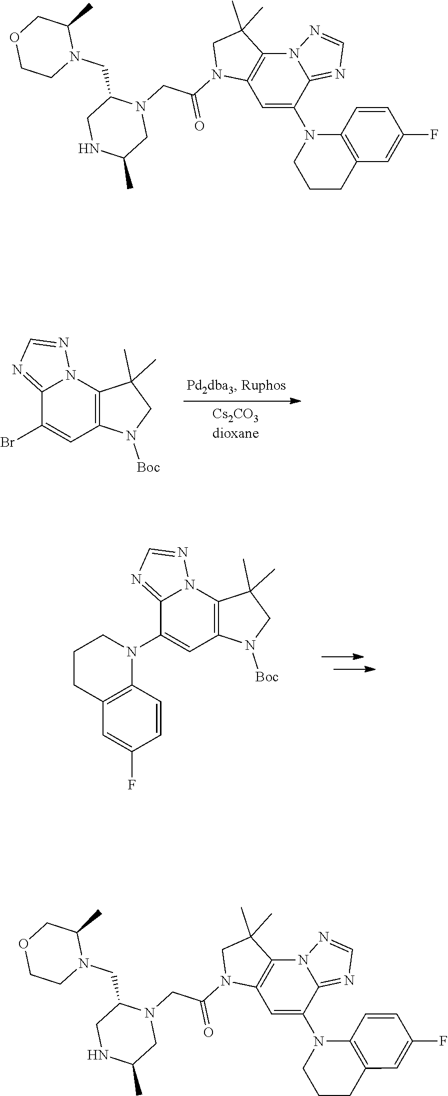 [SMILES: C[C@@H]1CN(CC(=O)N2CC(C)(C)c3c2cc(N2CCCc4cc(F)ccc42)c2ncnn32)[C@@H](CN2CCOC[C@H]2C)CN1 ; CC(C)(C)OC(=O)N1CC(C)(C)c2c1cc(Br)c1ncnn21 ; CC(C)(C)OC(=O)N1CC(C)(C)c2c1cc(N1CCCc3cc(F)ccc31)c1ncnn21 ; C[C@@H]1CN(CC(=O)N2CC(C)(C)c3c2cc(N2CCCc4cc(F)ccc42)c2ncnn32)[C@@H](CN2CCOC[C@H]2C)CN1]

Step 1: tert-butyl 4-(6-fluoro-3,4-dihydroquinolin-1(2H)-yl)-8,8-dimethyl-7,8-dihydro-6H-pyrrolo[2,3-e][1,2,4]triazolo[1,5-a]pyridine-6-carboxylate

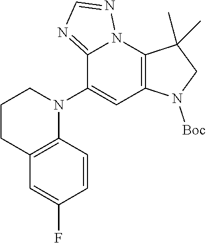 [SMILES: CC(C)(C)OC(=O)N1CC(C)(C)c2c1cc(N1CCCc3cc(F)ccc31)c1ncnn21]

A mixture of tert-butyl 4-bromo-8,8-dimethyl-7,8-dihydro-6H-pyrrolo[2,3-e][1,2,4]triazolo[1,5-a]pyridine-6-carboxylate (300 mg, 0.817 mmol, 1.0 eq), 6-fluoro-1,2,3,4-tetrahydroquinoline (148 mg, 0.980 mmol, 1.2 eq), Cs₂CO₃ (532 mg, 1.634 mmol, 2.0 eq), Pd₂(dba)₃ (74.8 mg, 0.082 mmol, 0.1 eq) and Ruphos (50 mg, 0.107 mmol, 0.13 eq) in dioxane (20 mL) was stirred at 100° C. for 16 h. The mixture was filtered and the filtrate was concentrated to give a yellow oil, which was purified by column eluted with 20% EA/PE to give the title compound (250 mg, 69.9%) as a yellow oil. MS (ESI) m/z 438.84 [M+H]⁺

Following steps: compound 168 was obtained as a yellow solid (40 mg) in a manner similar to that described in compound 14. ¹H NMR (400 MHz, DMSO-d⁶) δ (ppm) 8.49 (s, 1H), 8.30 (s, 1H), 7.01 (dd, J=9.4, 2.7 Hz, 1H), 6.80 (td, J=8.8, 2.9 Hz, 1H), 6.62 (dd, J=8.9, 5.0 Hz, 1H), 4.18 (d, J=10.4 Hz, 1H), 4.00 (d, J=10.6 Hz, 1H), 3.89-3.66 (m, 4H), 3.63-3.50 (m, 2H), 3.32-3.20 (m, 2H), 3.07-2.58 (m, 10H), 2.38-2.28 (m, 1H), 2.25-2.15 (m, 1H), 2.02-1.88 (m, 3H), 1.85-1.74 (m, 1H), 1.65 (d, J=17.1 Hz, 6H), 0.99-0.87 (m, 6H). MS (ESI) m/z 591.59 [M+H]⁺

Example 117: 1-(4-(7-fluoro-2,3-dihydro-4H-benzo[b][1,4]oxazin-4-yl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 169)

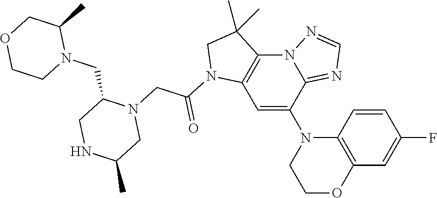 [SMILES: C[C@@H]1CN(CC(=O)N2CC(C)(C)c3c2cc(N2CCOc4cc(F)ccc42)c2ncnn32)[C@@H](CN2CCOC[C@H]2C)CN1]

Compound 169 was obtained as a yellow solid (54 mg) in a manner similar to that described in compound 168. ¹H NMR (400 MHz, DMSO-d⁶) δ (ppm) 8.54 (s, 1H), 8.27 (s, 1H), 6.94-6.83 (m, 2H), 6.68 (td, J=8.6, 2.8 Hz, 1H), 4.30-3.94 (m, 6H), 3.86-3.67 (m, 2H), 3.63-3.50 (m, 2H), 3.33-3.19 (m, 4H), 3.06-2.61 (m, 6H), 2.37-2.15 (m, 2H), 2.01-1.91 (m, 1H), 1.85-1.74 (m, 1H), 1.64 (d, J=17.2 Hz, 6H), 0.99-0.86 (m, 6H). MS (ESI) m/z 593.54 [M+H]⁺

Example 118: 1-(4-(4-fluorobenzyl)-2-(3-hydroxy-3-methylazetidin-1-yl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 170)

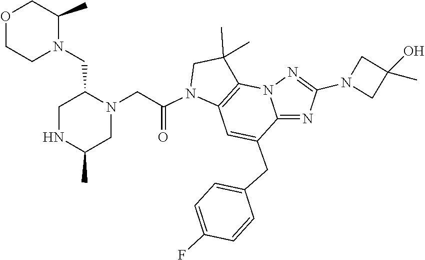 [SMILES: C[C@@H]1CN(CC(=O)N2CC(C)(C)c3c2cc(Cc2ccc(F)cc2)c2nc(N4CC(C)(O)C4)nn32)[C@@H](CN2CCOC[C@H]2C)CN1]

Following the synthesis of compound 195, compound 170 was obtained as a white solid. MS (ESI) m/z 635.6 [M+H]⁺. ¹H NMR (400 MHz, DMSO-d⁶) δ (ppm) 8.18 (s, 1H), 7.38 (dd, J=8.4, 5.6 Hz, 2H), 7.14 (t, J=8.8 Hz, 2H), 5.60 (s, 1H), 4.17 (d, J=3.3 Hz, 2H), 4.11 (d, J=10.8 Hz, 1H), 3.97-3.87 (m, 4H), 3.82 (d, J=16.6 Hz, 1H), 3.63 (d, J=16.6 Hz, 1H), 3.56-3.45 (m, 3H), 3.41-3.20 (m, 3H), 3.17 (t, J=10.5 Hz, 1H), 3.05-2.83 (m, 3H), 2.83-2.72 (m, 1H), 2.64 (d, J=9.8 Hz, 2H), 2.32 (t, J=11.3 Hz, 1H), 2.17 (s, 1H), 1.95 (t, J=10.4 Hz, 1H), 1.78 (d, J=12.8 Hz, 1H), 1.60 (s, 3H), 1.55 (s, 3H), 1.48 (s, 3H), 0.92 (dd, J=6.3, 2.9 Hz, 6H).

Example 119: 1-(4-(4-fluorobenzyl)-2-isopropyl-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 171)

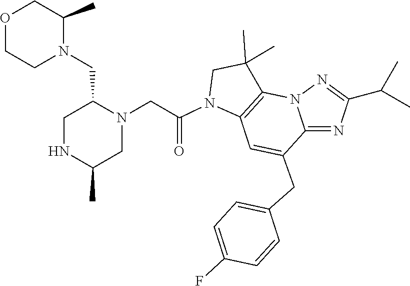 [SMILES: CC(C)c1nc2c(Cc3ccc(F)cc3)cc3c(n2n1)C(C)(C)CN3C(=O)CN1C[C@@H](C)NC[C@@H]1CN1CCOC[C@H]1C]

Compound 171 was obtained as a white solid after lyophilization in a manner similar to that described in compound 163. MS (ESI) m/z 592.6 [M+H]⁺. ¹H NMR (400 MHz, DMSO-d⁶) δ (ppm) 8.28 (s, 1H), 7.47-7.39 (m, 2H), 7.19-7.10 (m, 2H), 4.35-4.22 (m, 2H), 4.14 (d, J=10.6 Hz, 1H), 3.94 (d, J=10.6 Hz, 1H), 3.81 (d, J=16.7 Hz, 1H), 3.69 (d, J=16.7 Hz, 1H), 3.48 (t, J=8.8 Hz, 2H), 3.22 (p, J=6.9 Hz, 1H), 3.15 (t, J=9.9 Hz, 1H), 3.04 (d, J=9.5 Hz, 1H), 2.96-2.73 (m, 4H), 2.66 (d, J=9.9 Hz, 2H), 2.54 (s, 2H), 2.33 (t, J=11.1 Hz, 1H), 2.18 (s, 1H), 1.95 (t, J=10.2 Hz, 1H), 1.83-1.75 (m, 1H), 1.64 (s, 3H), 1.59 (s, 3H), 1.41 (s, 3H), 1.39 (s, 3H), 0.93 (d, J=6.1 Hz, 6H).

Example 120: 1-(1-ethyl-4-(4-fluorobenzyl)-8,8-dimethyl-7,8-dihydro-6H-pyrrolo[2,3-e][1,2,4]triazolo[4,3-a]pyridin-6-yl)-2-((2R,5R)-5-methyl-2-(((R)-3-m ethylmorpholino)methyl)piperazin-1-yl)ethan-1-one (compound 172)

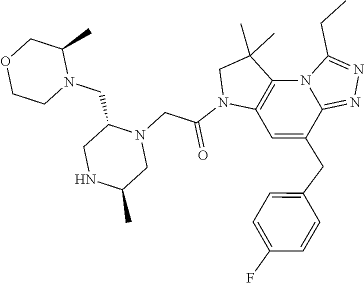 [SMILES: CCc1nnc2c(Cc3ccc(F)cc3)cc3c(n12)C(C)(C)CN3C(=O)CN1C[C@@H](C)NC[C@@H]1CN1CCOC[C@H]1C]

Compound 172 was obtained as a white solid (10.7 mg) in a manner similar to that described in compound 10. ¹H NMR (400 MHz, DMSO-d⁶) δ 8.18 (s, 1H), 7.41 (dd, J=8.5, 5.7 Hz, 2H), 7.11 (t, J=8.9 Hz, 2H), 4.27 (q, J=14.7 Hz, 2H), 4.08 (d, J=10.5 Hz, 1H), 3.90-3.77 (m, 2H), 3.61 (d, J=16.2 Hz, 1H), 3.48 (d, J=11.0 Hz, 2H), 3.24-3.12 (m, 4H), 2.94-2.73 (m, 4H), 2.64-2.58 (m, 2H), 2.48-2.39 (m, 1H), 2.34-2.25 (m, 1H), 2.22-2.12 (m, 1H), 1.93 (t, J=9.2 Hz, 1H), 1.76 (d, J=12.6 Hz, 1H), 1.57-1.47 (m, 9H), 0.93-0.87 (m, 6H). MS (ESI) m/z 578.6 [M+H]⁺.

Example 121: 2-((2R,5R)-2-(((3S,5R)-3,5-dimethylmorpholino)methyl)-5-methylpiperazin-1-yl)-1-(4-(4-fluorobenzyl)-8,8-dimethyl-2-(trifluoromethyl)-7,8-dihydro-6H-imidazo[1,2-a]pyrrolo[2,3-e]pyridin-6-yl)ethan-1-one (compound 173)

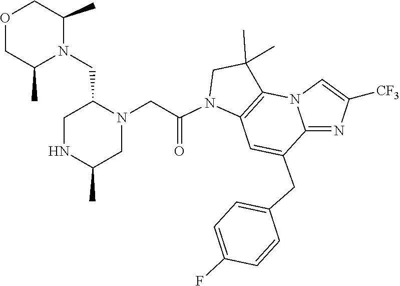 [SMILES: C[C@@H]1CN(CC(=O)N2CC(C)(C)c3c2cc(Cc2ccc(F)cc2)c2nc(C(F)(F)F)cn32)[C@@H](CN2[C@H](C)COC[C@@H]2C)CN1]

Following the synthesis of compound 146, compound 173 was obtained as a white solid (34 mg). ¹H NMR (400 MHz, DMSO-d⁶) δ (ppm) 8.68 (s, 1H), 8.15 (s, 1H), 7.38 (dd, J=8.4, 5.7 Hz, 2H), 7.12 (t, J=8.9 Hz, 2H), 4.28 (s, 2H), 4.15 (d, J=10.6 Hz, 1H), 4.03 (d, J=10.5 Hz, 1H), 3.78 (d, J=16.0 Hz, 1H), 3.54 (ddd, J=22.8, 11.0, 2.4 Hz, 2H), 3.23 (d, J=16.0 Hz, 1H), 3.15 (d, J=10.2 Hz, 1H), 3.06-2.96 (m, 2H), 2.72-2.56 (m, 4H), 2.39-2.12 (m, 5H), 1.54 (d, J=11.5 Hz, 6H), 0.87 (t, J=6.9 Hz, 9H). MS (ESI) m/z 631.6 [M+H]⁺.

Example 122: 2-((2R,5R)-2-(((R)-4-acetyl-2-methylpiperazin-1-yl)methy 1)-5-methylpiperazin-1-yl)-1-(4-(4-fluorobenzyl)-8,8-dimethyl-2-(trifluoromethyl)-7,8-dihydro-6H-imidazo[1,2-a]pyrrolo[2,3-e]pyridin-6-yl)ethan-1-one (compound 174)

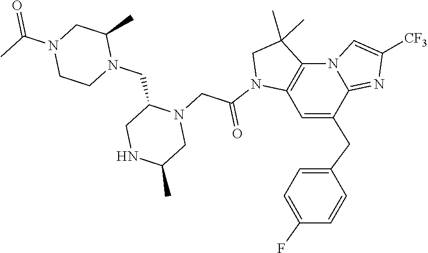 [SMILES: CC(=O)N1CCN(C[C@H]2CN[C@H](C)CN2CC(=O)N2CC(C)(C)c3c2cc(Cc2ccc(F)cc2)c2nc(C(F)(F)F)cn32)[C@H](C)C1]

Following the synthesis of compound 146, compound 174 was obtained as a white solid (36 mg). ¹H NMR (400 MHz, DMSO-d⁶) δ (ppm) 8.68 (s, 1H), 8.21 (d, J=5.4 Hz, 11H), 7.38 (dd, J=13.7, 7.7 Hz, 2H), 7.13 (t, J=8.8 Hz, 2H), 4.36-4.22 (m, 2H), 4.11 (d, J=10.6 Hz, 1H), 3.90 (d, J=10.6 Hz, 1H), 3.80 (dd, J=16.6, 4.7 Hz, 1H), 3.71-3.51 (m, 2H), 3.44 (d, J=12.1 Hz, 11H), 3.06-2.90 (m, 2H), 2.87-2.74 (m, 4H), 2.64 (d, J=8.9 Hz, 2H), 2.47-2.37 (m, 1H), 2.36-2.15 (m, 2H), 2.02-1.91 (m, 1H), 1.91-1.76 (m, 4H), 1.63-1.48 (m, 6H), 1.02-0.88 (m, 6H). MS (ESI) m/z 658.6 [M+H]⁺.

Example 123: 2-((R)-4-(((2R,5R)-1-(2-(4-(4-fluorobenzyl)-8,8-dimethyl-2-(trifluoromethyl)-7,8-dihydro-6H-imidazo[1,2-a]pyrrolo[2,3-e]pyridin-6-yl)-2-oxoethyl)-5-methylpiperazin-2-yl)methyl)-3-methylpiperazin-1-yl)acetonitrile (compound 175)

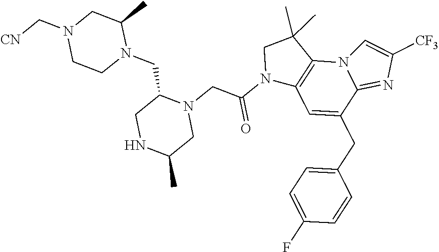

Following the synthesis of compound 146, compound 175 was obtained as a white solid (24 mg). ¹H NMR (400 MHz, DMSO-d⁶) δ (ppm) 8.59 (s, 1H), 8.16 (s, 1H), 7.36 (dd, J=8.4, 5.7 Hz, 2H), 7.14 (t, J=8.9 Hz, 2H), 4.28 (dd, J=40.3, 15.1 Hz, 2H), 4.06 (d, J=10.5 Hz, 1H), 3.86 (d, J=10.5 Hz, 1H), 3.74 (t, J=20.9 Hz, 2H), 3.49 (dd, J=38.6, 17.2 Hz, 2H), 3.09-3.00 (m, 1H), 2.97-2.82 (m, 2H), 2.67 (dd, J=30.9, 9.2 Hz, 3H), 2.58-2.52 (m, 1H), 2.49-2.44 (m, 1H), 2.36-2.24 (m, 2H), 2.24-2.11 (m, 1H), 2.09-1.96 (m, 1H), 1.96-1.78 (m, 2H), 1.74 (d, J=13.9 Hz, 1H), 1.56 (d, J=34.0 Hz, 6H), 0.98 (d, J=6.1 Hz, 3H), 0.89 (d, J=6.0 Hz, 3H). MS (ESI) m/z 655.6 [M+H]⁺.

Example 124: 1-(4-((4-fluorophenyl)sulfonyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 176)

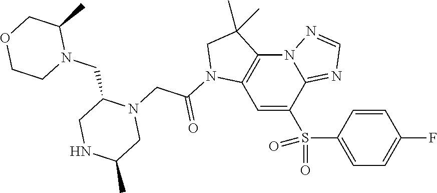 [SMILES: C[C@@H]1CN(CC(=O)N2CC(C)(C)c3c2cc(S(=O)(=O)c2ccc(F)cc2)c2ncnn32)[C@@H](CN2CCOC[C@H]2C)CN1]

Following the synthesis of compound 49, compound 176 was obtained as a white solid (50 mg).

NMR (400 MHz, DMSO-d⁶) δ (ppm) 9.29 (s, 1H), 8.72 (s, 1H), 8.28 (dd, J=8.9, 5.1 Hz, 2H), 7.51 (t, J=8.8 Hz, 2H), 4.27 (d, J=10.5 Hz, 1H), 4.05 (d, J=10.6 Hz, 1H), 3.96 (d, J=16.7 Hz, 1H), 3.71 (d, J=16.6 Hz, 1H), 3.64-3.49 (m, 2H), 3.30-3.20 (m, 2H), 3.10-2.66 (m, 7H), 2.51-2.46 (m, 1H), 2.41-2.33 (m, 1H), 2.26-2.16 (m, 1H), 2.05-1.96 (m, 1H), 1.87-1.80 (m, 1H), 1.64 (d, J=13.7 Hz, 6H), 0.99-0.91 (m, 6H). MS (ESI) m/z 600.56 [M+H]⁺

Example 125: 1-(4-(4-fluorobenzyl)-8,8-dimethyl-2-(trifluoromethyl)-7,8-dihydro-6H-imidazo[1,2-a]pyrrolo[2,3-e]pyridin-6-yl)-2-((2S,5R)-5-methyl-2-((2-(trifluoromethyl)-1H-imidazol-1-yl)methyl)piperazin-1-yl)ethan-1-one (compound 177)

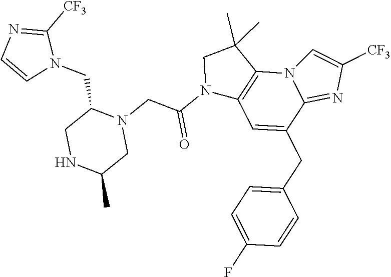 [SMILES: C[C@@H]1CN(CC(=O)N2CC(C)(C)c3c2cc(Cc2ccc(F)cc2)c2nc(C(F)(F)F)cn32)[C@@H](Cn2ccnc2C(F)(F)F)CN1]

Following the synthesis of compound 146, compound 177 was obtained as a white solid (13 mg). ¹H NMR (400 MHz, DMSO-d⁶) δ (ppm) 8.70 (s, 1H), 8.22 (s, 1H), 7.62 (s, 1H), 7.39 (dd, J=8.4, 5.7 Hz, 2H), 7.18-7.05 (m, 3H), 4.45 (d, J=10.7 Hz, 1H), 4.30 (s, 2H), 4.07 (q, J=10.6 Hz, 3H), 3.80 (d, J=16.5 Hz, 1H), 3.41-3.34 (m, 1H), 3.09-2.91 (m, 1H), 2.80 (d, J=10.6 Hz, 1H), 2.67-2.59 (m, 1H), 2.48-2.42 (m, 1H), 2.37-2.27 (m, 1H), 2.26-2.13 (m, 1H), 1.55 (d, J=4.5 Hz, 6H), 0.88 (d, J=5.8 Hz, 3H). MS (ESI) m/z 652.4 [M+H]⁺.

Example 126: 1-(4-(4-fluorobenzyl)-8,8-dimethyl-2-(trifluoromethyl)-7,8-dihydro-6H-imidazo[1,2-a]pyrrolo[2,3-e]pyridin-6-yl)-2-((2R,5R)-5-methyl-2-(((R)-2-methyl-4-(methylsulfonyl)piperazin-1-yl)methyl)piperazin-1-yl)ethan-1-one (compound 178)

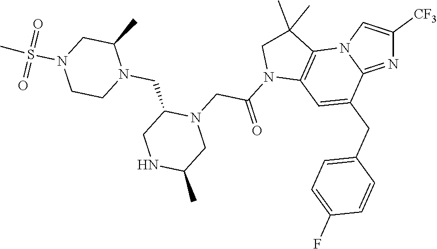 [SMILES: C[C@@H]1CN(CC(=O)N2CC(C)(C)c3c2cc(Cc2ccc(F)cc2)c2nc(C(F)(F)F)cn32)[C@@H](CN2CCN(S(C)(=O)=O)C[C@H]2C)CN1]

Following the synthesis of compound 146, compound 178 was obtained as a white solid (22 mg). ¹H NMR (400 MHz, DMSO-d⁶) δ (ppm) 8.68 (s, 1H), 8.18 (s, 1H), 7.38 (t, J=6.8 Hz, 2H), 7.12 (t, J=8.7 Hz, 2H), 4.35-4.21 (m, 2H), 4.16 (d, J=10.6 Hz, 1H), 3.96-3.84 (m, 2H), 3.46-3.33 (m, 3H), 3.11 (t, J=11.0 Hz, 2H), 2.91-2.70 (m, 4H), 2.69-2.59 (m, 2H), 2.56 (s, 3H), 2.43-2.36 (m, 1H), 2.36-2.20 (m, 2H), 2.13 (t, J=9.1 Hz, 1H), 1.89 (d, J=11.9 Hz, 1H), 1.54 (d, J=15.0 Hz, 6H), 1.00 (d, J=5.9 Hz, 3H), 0.88 (d, J=4.5 Hz, 3H). MS (ESI) m/z 694.6 [M+H]⁺.

Example 127: 1-(4-(4-fluorobenzyl)-8,8-dimethyl-2-(trifluoromethyl)-7,8-dihydro-6H-imidazo[1,2-a]pyrrolo[2,3-e]pyridin-6-yl)-2-((2R,5R)-5-methyl-2-(((R)-2-methyl-4-(2,2,2-trifluoroethyl)piperazin-1-yl)methyl)piperazin-1-yl)ethan-1-one (compound 179)

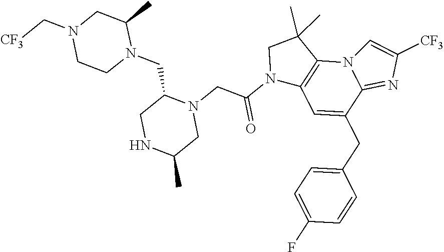 [SMILES: C[C@@H]1CN(CC(=O)N2CC(C)(C)c3c2cc(Cc2ccc(F)cc2)c2nc(C(F)(F)F)cn32)[C@@H](CN2CCN(CC(F)(F)F)C[C@H]2C)CN1]

Following the synthesis of compound 146, compound 179 was obtained as a white solid (36 mg). MS (ESI) m/z 698.7 [M+H]⁺.

Example 128: 1-(4′-(4-fluorobenzyl)-2,3,5,6-tetrahydrospiro[pyran-4,8′-pyrrolo[2,3-e][1,2,4]triazolo[1,5-a]pyridin]-6′(7′H)-yl)-2-((2R,5R)-5-methyl-2-(((R)-3-methylmorpholino)methyl)piperazin-1-yl)ethan-1-one (compound 180)

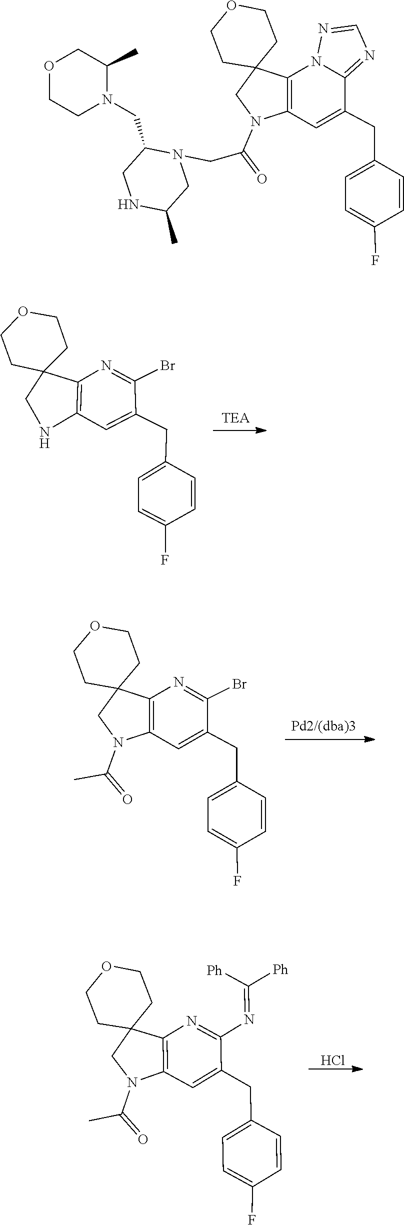 [SMILES: C[C@@H]1CN(CC(=O)N2CC3(CCOCC3)c3c2cc(Cc2ccc(F)cc2)c2ncnn32)[C@@H](CN2CCOC[C@H]2C)CN1 ; Fc1ccc(Cc2cc3c(nc2Br)C2(CCOCC2)CN3)cc1 ; CC(=O)N1CC2(CCOCC2)c2nc(Br)c(Cc3ccc(F)cc3)cc21 ; CC(=O)N1CC2(CCOCC2)c2nc(N=C(c3ccccc3)c3ccccc3)c(Cc3ccc(F)cc3)cc21 ; Cl]

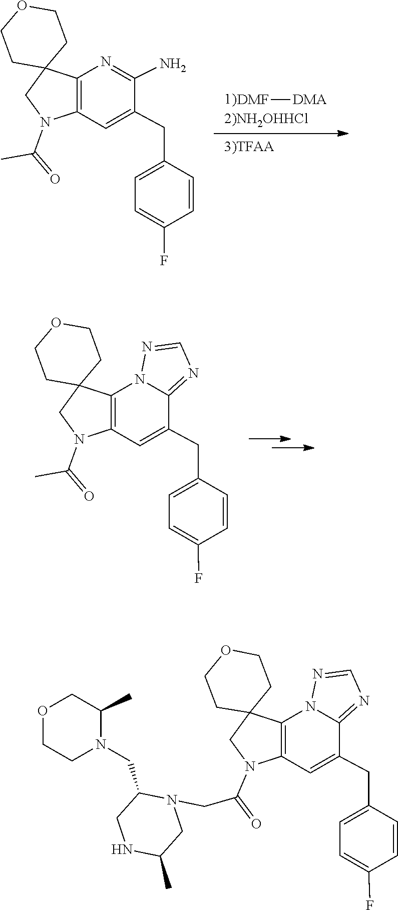

Step 1: 1-(5′-bromo-6′-(4-fluorobenzyl)-2,3,5,6-tetrahydrospiro[pyran-4,3′-pyrrolo[3,2-b]pyridin]-1′(2′H)-yl)ethan-1-one

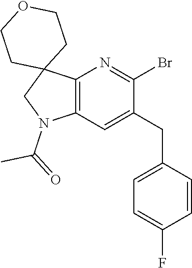 [SMILES: CC(=O)N1CC2(CCOCC2)c2nc(Br)c(Cc3ccc(F)cc3)cc21]

In a flame-dried 100 mL round-bottomed flask, 5′-bromo-6′-(4-fluorobenzyl)-1′,2,2′,3,5,6-hexahydrospiro[pyran-4,3′-pyrrolo[3,2-b]pyridine](1 g, 2.65 mmol, 1.0 eq) and N-ethyl-N-isopropylpropan-2-amine (0.514 g, 3.98 mmol, 1.5 eq) and acetyl chloride (0.208 g, 2.65 mmol, 1.0 eq) were dissolved in DCM (20 mL). The mixture was stirred for 1 h. The mixture was quenched by sat·NaHCO₃·aq. and diluted with dichloromethane (10 mL) and then washed with H₂O, and brine. The organic layer was dried over Na₂SO₄, filtered and concentrated. The crude product was added to a silica gel column and was eluted with EA/PE from 0˜30% to give the title compound (1.1 g, 99%) as a white solid. MS (ESI) m/z 420.9[M+H]⁺.

Step 2: 1-(5′-((diphenylmethylene)amino)-6′-(4-fluorobenzyl)-2,3,5,6-tetrahydrospiro[pyran-4,3′-pyrrolo[3,2-b]pyridin]-1′(2′H)-yl)ethan-1-one

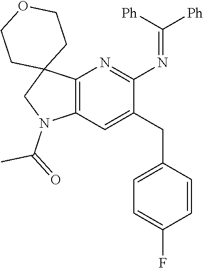 [SMILES: CC(=O)N1CC2(CCOCC2)c2nc(N=C(c3ccccc3)c3ccccc3)c(Cc3ccc(F)cc3)cc21]

In a flame-dried 50 mL round-bottomed flask, 1-(5′-bromo-6′-(4-fluorobenzyl)-2,3,5,6-tetrahydrospiro[pyran-4,3′-pyrrolo[3,2-b]pyri din]-1′(2′H)-yl)ethan-1-one (500 mg, 1.192 mmol, 1.0 eq), diphenylmethanimine (259 mg, 1.431 mmol, 1.2 eq), Pd2(dba)3 (109 mg, 0.119 mmol, 0.1 eq), BINAP (74.3 mg, 0.119 mmol, 0.1 eq) and Cs₂CO₃ (777 mg, 2.385 mmol, 2.0 eq) were dissolved in dioxane (15 mL) under N₂. The result mixture was stirred at 100° C. for 16 h. H₂O (15 ml) was added to the reaction mixture followed by extraction with ethyl acetate (8 ml×3). The combined organic layers were washed with brine, dried over Na₂SO₄, filtered and concentrated. The crude product was added to a silica gel column and was eluted with EA/PE from 0˜33% to give the title compound (440 mg, 80.0%) as a yellow solid. MS (ESI) m/z 520.3 [M+H]⁺.

Step 3: 1-(5′-amino-6′-(4-fluorobenzyl)-2,3,5,6-tetrahydrospiro[pyran-4,3′-pyrrolo[3,2-b]pyri din]-1′(2′H)-yl)ethan-1-one

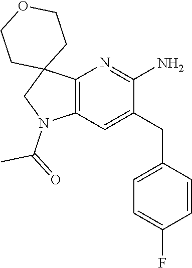 [SMILES: CC(=O)N1CC2(CCOCC2)c2nc(N)c(Cc3ccc(F)cc3)cc21]

In a flame-dried 50 mL round-bottomed flask, 1-(5′-((diphenylmethylene)amino)-6′-(4-fluorobenzyl)-2,3,5,6-tetrahydrospiro[pyran-4,3′-pyrrolo[3,2-b]pyridin]-1′(2′H)-yl)ethan-1-one (400 mg, 0.770 mmol, 1.0 eq) in MeOH (10 mL) was added 2N HCl (2 mL). The result mixture was stirred for 2 h. The mixture was concentrated and then extracted with ethyl acetate (8 ml×3). The organic layer was washed with saturated NaHCO₃, dried over Na₂SO₄, filtered and concentrated. The crude product was added to a silica gel column and was eluted with EA/PE from 0˜50% to give the title compound as an off-white solid. MS (ESI) m/z 356.8 [M+H]⁺.

Step 4: 1-(4′-(4-fluorobenzyl)-2,3,5,6-tetrahydrospiro[pyran-4,8′-pyrrolo[2,3-e][1,2,4]triazolo [1,5-a]pyridin]-6′(7′H)-yl)ethan-1-one

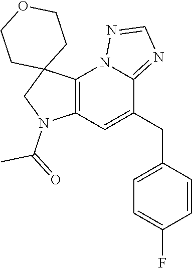 [SMILES: CC(=O)N1CC2(CCOCC2)c2c1cc(Cc1ccc(F)cc1)c1ncnn21]

In a flame-dried 50 mL round-bottomed flask, 1-(5′-amino-6′-(4-fluorobenzyl)-2,3,5,6-tetrahydrospiro[pyran-4,3′-pyrrolo[3,2-b]pyri din]-1′(2′H)-yl)ethan-1-one (120 mg, 0.338 mmol, 1.0 eq) and DMF-DMA (402 mg, 3.38 mmol, 10.0 eq) were dissolved in 2-propanol (5 mL). The reaction was stirred for 2 h at 100° C. and then cooled to 50° C. Hydroxylamine hydrochloride (117 mg, 1.688 mmol, 5.0 eq) was added to the reaction mixture. The result mixture was stirred for 1 h and concentrated. The reaction mixture was diluted with THF (10 ml). TFAA (284 mg, 1.351 mmol, 4.0 eq) was added to the reaction mixture. The reaction was stirred for 14 h. The reaction mixture was diluted with ethyl acetate (20 mL) and then washed with saturated NaHCO₃, brine. The organic layer was dried over Na₂SO₄, filtered and concentrated. The crude product was added to a silica gel column and was eluted with PE/EA from 0˜50% to give the title compound (100 mg, 78%) as a colorless oil. MS (ESI) m/z 381.8 [M+H]⁺.

Following Steps: following the synthesis of compound 14, compound 180 was obtained as a white solid after lyophilization. MS (ESI) m/z 592.6 [M+H]⁺. ¹H NMR (400 MHz, DMSO-d6) δ (ppm) 8.56 (d, J=1.1 Hz, 1H), 8.44 (s, 1H), 7.43 (dd, J=8.3, 5.6 Hz, 2H), 7.14 (t, J=8.7 Hz, 2H), 4.52 (d, J=10.8 Hz, 1H), 4.33 (d, J=2.9 Hz, 2H), 4.16 (d, J=10.8 Hz, 1H), 3.97 (d, J=15.6 Hz, 2H), 3.67 (d, J=16.6 Hz, 1H), 3.63-3.47 (m, 4H), 3.23 (t, J=10.3 Hz, 1H), 3.00-2.88 (m, 3H), 2.87-2.58 (m, 5H), 2.52-2.50 (m, 3H), 2.42 (t, J=10.4 Hz, 1H), 2.32 (t, J=10.7 Hz, 1H), 2.22 (s, 1H), 2.01 (t, J=10.3 Hz, 1H), 1.83 (d, J=11.1 Hz, 1H), 1.54 (t, J=12.2 Hz, 2H), 0.94 (d, J=6.2 Hz, 3H), 0.91 (d, J=6.0 Hz, 3H).

Example 129: 1-(4-(4-fluorobenzyl)-8,8-dimethyl-2-(trifluoromethyl)-7,8-dihydro-6H-imidazo[1,2-a]pyrrolo[2,3-e]pyridin-6-yl)-2-((2R,5R)-5-methyl-2-(((R)-2-methyl-4-(oxetan-3-yl)piperazin-1-yl)methyl)piperazin-1-yl)ethan-1-one (compound 181)

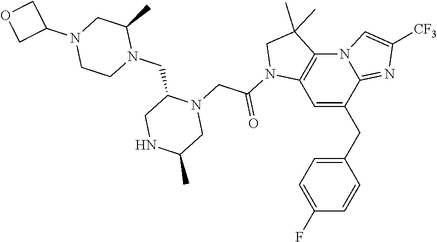 [SMILES: C[C@@H]1CN(CC(=O)N2CC(C)(C)c3c2cc(Cc2ccc(F)cc2)c2nc(C(F)(F)F)cn32)[C@@H](CN2CCN(C3COC3)C[C@H]2C)CN1]

Following the synthesis of compound 116, compound 181 was obtained as a white solid (32 mg). ¹H NMR (400 MHz, DMSO-d6) δ (ppm) 8.69 (s, 1H), 8.20 (s, 1H), 7.38 (dd, J=8.3, 5.7 Hz, 2H), 7.13 (t, J=8.8 Hz, 2H), 4.38-4.17 (m, 6H), 4.10 (d, J=10.5 Hz, 1H), 3.94-3.77 (m, 2H), 3.54 (d, J=16.4 Hz, 1H), 3.08-2.98 (m, 1H), 2.93-2.77 (m, 3H), 2.71 (d, J=10.7 Hz, 1H), 2.60 (d, J=8.6 Hz, 2H), 2.46-2.32 (m, 2H), 2.31-2.23 (m, 2H), 2.25-2.10 (m, 1H), 2.04-1.83 (m, 2H), 1.75 (d, J=11.0 Hz, 2H), 1.55 (d, J=13.4 Hz, 6H), 0.97 (d, J=6.1 Hz, 3H), 0.87 (d, J=6.0 Hz, 3H). MS (ESI) m/z 671.6 [M+H]⁺.

Example 130: 2-((R)-4-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)-3-methylpiperazin-1-yl)acetonitrile (compound 182)

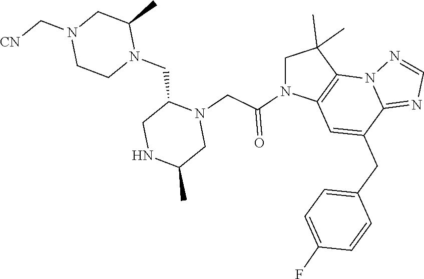

Following the synthesis of compound 116, compound 182 was obtained as a white solid (20 mg). ¹H NMR (400 MHz, DMSO-d⁶) δ (ppm) 8.43 (s, 1H), 8.35 (s, 1H), 7.38 (dd, J=8.5, 5.7 Hz, 2H), 7.12 (t, J=8.9 Hz, 2H), 4.29 (q, J=14.8 Hz, 2H), 4.11 (d, J=10.5 Hz, 1H), 3.91 (d, J=10.5 Hz, 1H), 3.80 (d, J=16.8 Hz, 1H), 3.67 (d, J=16.6 Hz, 1H), 3.48 (dd, J=37.7, 17.2 Hz, 2H), 3.01 (d, J=8.8 Hz, 1H), 2.98-2.78 (m, 2H), 2.71-2.57 (m, 3H), 2.48-2.44 (m, 1H), 2.37-2.16 (m, 3H), 2.09-1.81 (m, 4H), 1.73 (d, J=11.8 Hz, 1H), 1.64 (s, 3H), 1.55 (s, 3H), 0.99 (d, J=6.1 Hz, 3H), 0.87 (d, J=5.9 Hz, 3H). MS (ESI) m/z 588.6 [M+H]⁺.

Example 131: 1-(4-(4-fluorobenzyl)-2-(trifluoromethyl)-2′,3′,5′,6′-tetrahydrospiro[imidazo[1,2-a]pyrrolo[2,3-e]pyridine-8,4′-pyran]-6(7H)-yl)-2-((2R,5R)-5-methyl-2-(((R)-3-methylmorpholino)methyl)piperazin-1-yl)ethan-1-one (compound 183)

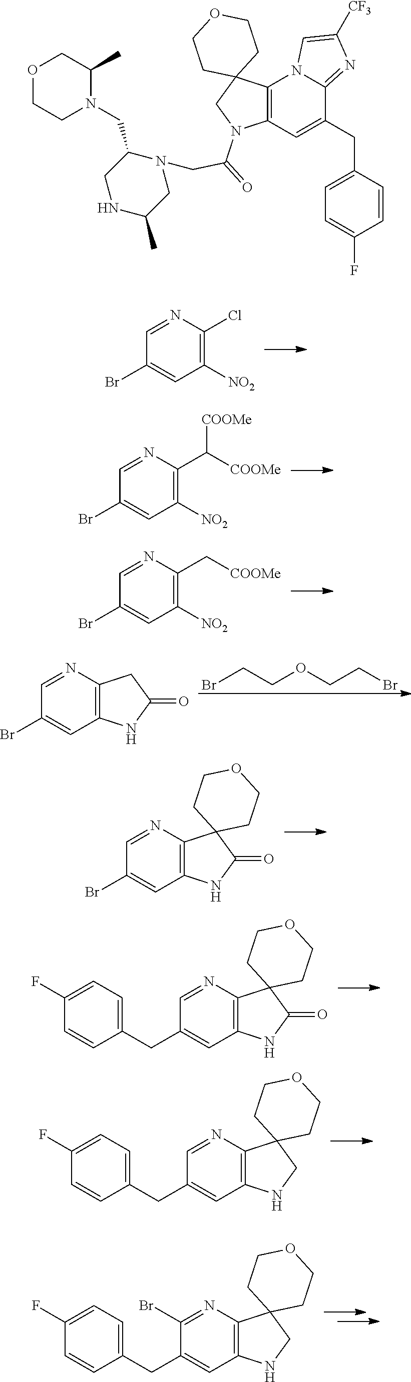

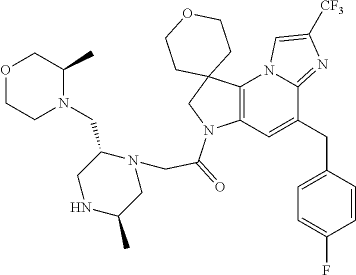 [SMILES: C[C@@H]1CN(CC(=O)N2CC3(CCOCC3)c3c2cc(Cc2ccc(F)cc2)c2nc(C(F)(F)F)cn32)[C@@H](CN2CCOC[C@H]2C)CN1]

Step 1: dimethyl 2-(5-bromo-3-nitropyridin-2-yl)malonate

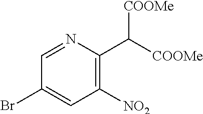 [SMILES: COC(=O)C(C(=O)OC)c1ncc(Br)cc1[N+](=O)[O-]]

To a solution of K₂CO₃ (175 g, 3.0 eq.) in 700 mL of K₂CO₃ was added 5-bromo-2-chloro-3-nitropyridine (175 g, 3.0 eq.) at 0° C. slowly. The reaction mixture was stirred at rt for 18 hours. LC-MS showed the reaction was completed. The residue was diluted with water and extracted with ethyl acetate. The combined organic layers were washed with brine, dried with anhydrous sodium sulfate, filtered and concentrated in vacuum. The residue was purified by silica gel chromatography to give the title compound (126 g, 90%).

Step 2: methyl 2-(5-bromo-3-nitropyridin-2-yl)acetate

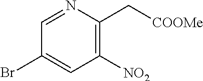 [SMILES: COC(=O)Cc1ncc(Br)cc1[N+](=O)[O-]]

To a solution of above intermediate (162 g, 1.0 eq.) in DMSO (500 mL) and H₂O (25 mL) was added NaCl (80 g, 2.0 eq.). The reaction mixture was stirred at 120° C. for 2 hours. LC-MS showed the reaction was completed. The residue was diluted with water and extracted with ethyl acetate. The combined organic layers were washed with brine, dried with anhydrous sodium sulfate, filtered and concentrated in vacuum. The residue was purified by silica gel chromatography to give the title compound (110 g, 60%).

Step 3: 6-bromo-1,3-dihydro-2H-pyrrolo[3,2-b]pyridin-2-one

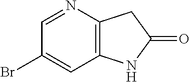 [SMILES: O=C1Cc2ncc(Br)cc2N1]

To a solution of above intermediate (30 g, 1.0 eq.) in EtOH (300 mL) and conc. HCl (65 mL) was added iron powder (18.5 g, 3 eq.). The reaction mixture was stirred at 85° C. for 1.5 hours. LC-MS showed the reaction was completed. The residue was filtered, diluted with water and extracted with ethyl acetate. The combined organic layers were dried over with anhydrous sodium sulfate, filtered and concentrated in vacuum. The residue was purified by silica gel chromatography to give the title compound (18 g, 69% yield). HPLC-MS: [M+H]⁺=213.20/215.20 found.

Step 4: 6′-bromo-2,3,5,6-tetrahydrospiro[pyran-4,3′-pyrrolo[3,2-b]pyridin]-2′(1′H)-one

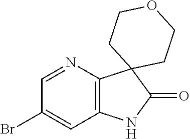 [SMILES: O=C1Nc2cc(Br)cnc2C12CCOCC2]

To a solution of above intermediate (14.6 g, 6.86 mmol, 1.0 eq) in dry THF (40 mL) was added LiHMDS (205 mL, 20.6 mmol, 3.0 eq) slowly at −78° C. Then mixture was stirred 30 min. 1-bromo-2-(2-bromoethoxy)ethane (31.8 g, 13.7 mmol, 2.0 eq) was added. The mixture was warmed to rt and stirred for 2 h, then the mixture was heated to 50° C. for 4 h. The mixture was quenched by NH₄Cl solution, diluted by ethyl acetate (200 mL). The organic phase was collected, dried by anhydrous Na₂SO₄. After concentration, the residue was purified by silica-gel column chromatography to give the title compound (3.0 g, 15.5%). UPLC-MS: [M+H]⁺=283.20/285.20 found.

Step 5: 6′-(4-fluorobenzyl)-2,3,5,6-tetrahydrospiro[pyran-4,3′-pyrrolo[3,2-b]pyridin]-2′(1′H)-one

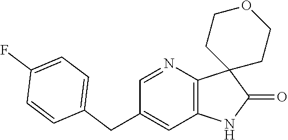 [SMILES: O=C1Nc2cc(Cc3ccc(F)cc3)cnc2C12CCOCC2]

A vial was charged with above intermediate (2.5 g, 1.0 eq), 2-(4-fluorophenyl)-4,4,5,5-tetramethyl-1,3,2-dioxaborolane (10.5 g, 5.0 eq), Pd(dppf)Cl₂ (1.29 g, 0.2 eq), Cs₂CO₃ (17.2 g, 6.0 eq) and dioxane/H₂O (5/1, 60 mL). The mixture was purged with nitrogen and heated to 100′C for 2 h. TLC showed the reaction was complete. The mixture was diluted with 150 mL H₂O and extracted with ethyl acetate and dried over sodium sulfate. The residue was filtered, concentrated in vacuum and further purified by silica gel chromatography to give the title compound (1.8 g, 65%). HPLC-MS: [M+H]⁺=313.20.

Step 6: 6′-(4-fluorobenzyl)-1′,2,2′,3,5,6-hexahydrospiro[pyran-4,3′-pyrrolo[3,2-b]pyridine]

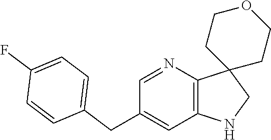 [SMILES: Fc1ccc(Cc2cnc3c(c2)NCC32CCOCC2)cc1]

To a solution of above intermediate (2.3 g, 1.0 eq.) in dry THF (30 mL) was added LiAlH₄ (0.98 g, 3.5 eq.) in three portions at −40° C. under nitrogen. The reaction mixture was stirred at −40° C. for 10 min, then was heated to 60° C. for 1 hours. LC-MS showed the reaction was completed. The reaction mixture was added slowly the water at −40° C. and 10% NaOH in water, dried with anhydrous sodium sulfate, filtered and concentrated in vacuum. The residue was purified by silica gel chromatography to give the title compound (0.9 g, 41%). HPLC-MS: [M+H]⁺=299.20.

Step 7: 5′-bromo-6′-(4-fluorobenzyl)-1′,2,2′,3,5,6-hexahydrospiro[pyran-4,3′-pyrrolo[3,2-b]pyridine]

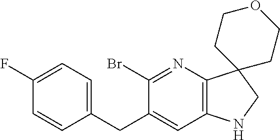 [SMILES: Fc1ccc(Cc2cc3c(nc2Br)C2(CCOCC2)CN3)cc1]

To a solution of above intermediate (1.6 g, 1.0 eq.) in DMF (15 mL) was slowly added the mixture of NBS (0.86 g, 0.9 eq.) and DMF (10 mL) at −30° C. under nitrogen. The reaction mixture was stirred at −30° C. for 1 hours. LC-MS showed the reaction was completed. The residue was diluted with water and extracted with ethyl acetate. The combined organic layers were washed with brine, dried with anhydrous sodium sulfate, filtered and concentrated in vacuum. The residue was purified by silica gel chromatography to give the title compound (1.6 g, 80%). ¹H NMR (400 MHz, chloroform-d) δ (ppm) 7.21-7.14 (m, 2H), 7.04-6.98 (m, 2H), 6.56 (s, 1H), 4.07 (dt, J=11.8, 4.1 Hz, 2H), 3.96 (s, 2H), 2.21-2.11 (m, 2H), 1.61 (dq, J=13.6, 2.1 Hz, 2H). HPLC-MS: [M+H]⁺=377.22/379.22 found.

Following steps: following the synthesis of compound 14 and compound 116, compound 183 was obtained as a white solid after lyophilization. MS (ESI) m/z 659.6 [M+H]⁺. ¹H NMR (400 MHz, DMSO-d⁶) δ (ppm) 8.75 (s, 1H), 8.26 (s, 1H), 7.45-7.36 (m, 2H), 7.21-7.10 (m, 2H), 4.49 (d, J=10.8 Hz, 1H), 4.32 (d, J=7.4 Hz, 2H), 4.11 (d, J=10.8 Hz, 1H), 4.07-3.84 (m, 3H), 3.71-3.50 (m, 3H), 3.49 (dd, J=11.1, 3.2 Hz, 2H), 3.37 (s, 2H), 3.22 (t, J=10.1 Hz, 1H), 3.02-2.59 (m, 6H), 2.54-2.56 (m, 2H), 2.42 (t, J=10.8 Hz, 1H), 2.31 (t, J=10.8 Hz, 1H), 2.21 (s, 1H), 2.04-1.94 (m, 1H), 1.82 (d, J=12.1 Hz, 1H), 1.53 (t, J=11.7 Hz, 2H), 0.93 (d, J=6.2 Hz, 3H), 0.90 (d, J=6.0 Hz, 3H).

Example 132: 1-(4-(4-fluorobenzyl)-8,8-dimethyl-2-(pyrimidin-5-yl)-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 184)

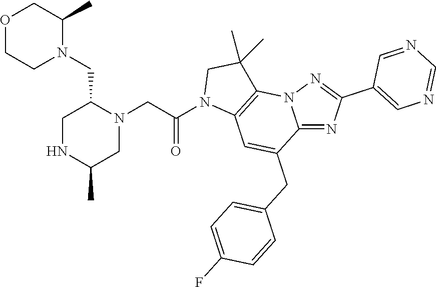 [SMILES: C[C@@H]1CN(CC(=O)N2CC(C)(C)c3c2cc(Cc2ccc(F)cc2)c2nc(-c4cncnc4)nn32)[C@@H](CN2CCOC[C@H]2C)CN1]

MS (ESI) m/z 628.6 [M+H]⁺. ¹H NMR (400 MHz, DMSO-d6) δ (ppm) 9.55 (s, 2H), 9.37 (s, 1H), 8.46 (s, 1H), 7.54-7.46 (m, 2H), 7.22-7.11 (m, 2H), 4.46-4.33 (m, 2H), 4.22 (d, J=10.5 Hz, 1H), 4.01 (d, J=10.6 Hz, 1H), 3.89 (d, J=16.6 Hz, 1H), 3.67 (d, J=16.5 Hz, 1H), 3.51 (d, J=11.2 Hz, 2H), 3.19 (t, J=10.3 Hz, 1H), 3.01-2.81 (m, 3H), 2.74 (dd, J=11.6, 2.7 Hz, 1H), 2.69-2.61 (m, 2H), 2.56-2.53 (m, 2H), 2.50-2.42 (m, 1H), 2.36-2.27 (m, 1H), 2.18 (s, 1H), 1.96 (t, J=10.5 Hz, 1H), 1.83-1.75 (m, 1H), 1.72 (s, 3H), 1.67 (s, 3H), 0.94 (d, J=6.2 Hz, 3H), 0.91 (d, J=6.0 Hz, 3H).

Example 133: 2-(4-(4-fluorobenzyl)-8,8-dimethyl-6-(2-((2R,5R)-5-methyl-2-(((R)-3-methylmorpholino)methyl)piperazin-1-yl)acetyl)-1-oxo-7,8-dihydro-1H-pyrrolo[2,3-e][1,2,4]triazolo[4,3-a]pyridin-2(6H)-yl)acetonitrile (compound 185)

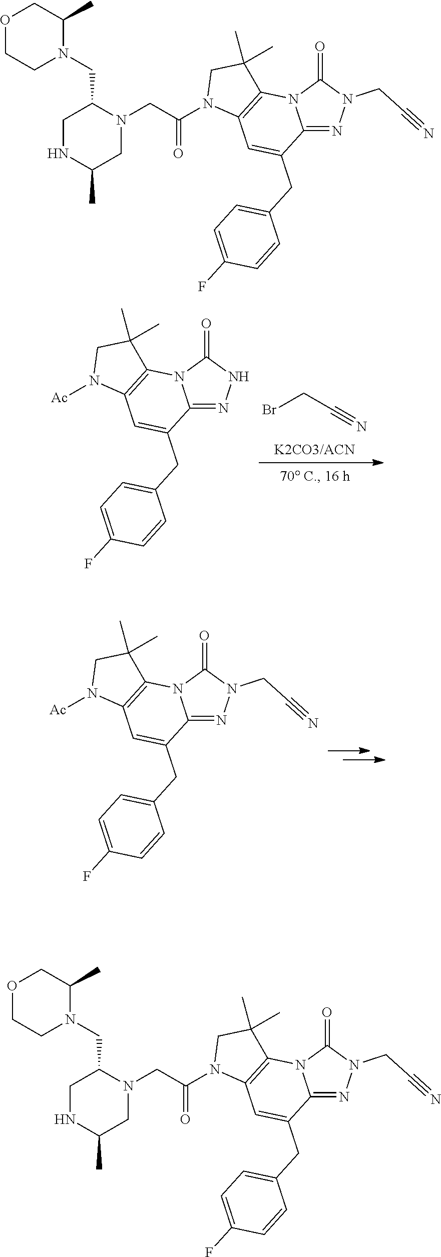

Step 1: 2-(6-acetyl-4-(4-fluorobenzyl)-8,8-dimethyl-1-oxo-7,8-dihydro-1H-pyrrolo[2,3-e][1,2,4]triazolo[4,3-a]pyridin-2(6H)-yl)acetonitrile

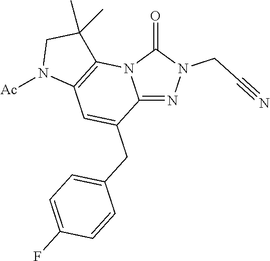 [SMILES: CC(=O)N1CC(C)(C)c2c1cc(Cc1ccc(F)cc1)c1nn(CC#N)c(=O)n21]

6-Acetyl-4-(4-fluorobenzyl)-8,8-dimethyl-2,6,7,8-tetrahydro-1H-pyrrolo[2,3-e][1,2,4]triazolo[4,3-a]pyridin-1-one (0.06 g, 0.169 mmol, 1.0 eq), 2-bromoacetonitrile (0.041 g, 0.339 mmol, 2.0 eq), and K₂CO₃ (0.070 g, 0.508 mmol, 3.0 eq) were dissolved in Acetonitrile (10 mL) under argon to give a yellow suspension. The reaction mixture was stirred at 70° C. for 16 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 80% to give the product (0.054 g, 81%) as a yellow oil. MS (ESI) m/z 393.4 [M+H]⁺.

Following steps: following the synthesis of compound 14, compound 185 was obtained as a white solid (11.1 mg). ¹H NMR (400 MHz, DMSO-d⁶) δ (ppm) 8.03 (s, 1H), 7.35 (dd, J=8.5, 5.6 Hz, 2H), 7.14 (t, J=8.9 Hz, 2H), 5.24 (s, 2H), 4.01-3.96 (m, 3H), 3.84-3.77 (m, 1H), 3.73 (d, J=16.7 Hz, 1H), 3.60 (s, 1H), 3.50 (d, J=10.9 Hz, 2H), 3.23-3.14 (m, 2H), 3.01-2.89 (m, 2H), 2.87-2.69 (m, 2H), 2.68-2.65 (m, 1H), 2.64-2.56 (m, 2H), 2.35-2.24 (m, 2H), 2.18-2.08 (m, 1H), 1.74 (d, J=11.4 Hz, 1H), 1.57 (d, J=11.8 Hz, 6H), 0.90-0.87 (m, 6H). MS (ESI) m/z 605.7 [M+H]⁺.

Example 134: 1-(4-(4-fluorobenzyl)-8,8-dimethyl-2-(trifluoromethyl)-7,8-dihydro-6H-imidazo[1,2-a]pyrrolo[2,3-e]pyridin-6-yl)-2-((2R,5R)-5-methyl-2-((2-methyl-1H-imidazol-1-yl)methyl)piperazin-1-yl)ethan-1-one (compound 186)

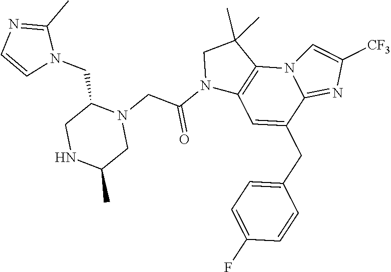 [SMILES: Cc1nccn1C[C@H]1CN[C@H](C)CN1CC(=O)N1CC(C)(C)c2c1cc(Cc1ccc(F)cc1)c1nc(C(F)(F)F)cn21]

Following the synthesis of compound 146, compound 186 was obtained as a white solid (8 mg). MS (ESI) m/z 598.6 [M+H]⁺.

Example 135: 1-(4-(4-fluorobenzyl)-8,8-dimethyl-2-(oxetan-3-ylamino)-7,8-dihydro-6H-pyrrolo[2,3-e][1,2,4]triazolo[1,5-a]pyridin-6-yl)-2-((2R,5R)-5-meth yl-2-(((R)-3-methylmorpholino)methyl)piperazin-1-yl)ethan-1-one (compound 187)

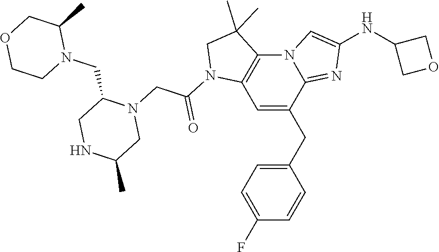 [SMILES: C[C@@H]1CN(CC(=O)N2CC(C)(C)c3c2cc(Cc2ccc(F)cc2)c2nc(NC4COC4)cn32)[C@@H](CN2CCOC[C@H]2C)CN1]

Following the synthesis of compound 195, compound 187 was obtained as a white solid. MS (ESI) m/z 621.6 [M+H]⁺.

Example 136: 1-(2-((3R,4R)-3,4-dihydroxypyrrolidin-1-yl)-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 188)

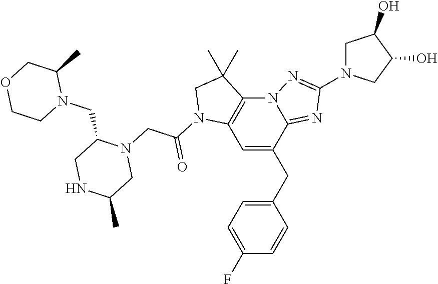 [SMILES: C[C@@H]1CN(CC(=O)N2CC(C)(C)c3c2cc(Cc2ccc(F)cc2)c2nc(N4C[C@@H](O)[C@H](O)C4)nn32)[C@@H](CN2CCOC[C@H]2C)CN1]

Following the synthesis of compound 195, compound 188 was obtained as a white solid. MS (ESI) m/z 651.6 [M+H]⁺. ¹H NMR (400 MHz, DMSO-d⁶) δ (ppm) 8.15 (s, 1H), 7.45-7.36 (m, 2H), 7.13 (t, J=8.9 Hz, 2H), 5.13 (d, J=3.2 Hz, 2H), 4.17 (d, J=3.9 Hz, 2H), 4.11 (d, J=10.9 Hz, 1H), 4.06 (d, J=3.8 Hz, 2H), 3.91 (d, J=10.6 Hz, 1H), 3.82 (d, J=16.5 Hz, 1H), 3.66 (d, J=3.9 Hz, 1H), 3.64 (d, J=3.5 Hz, 2H), 3.54-3.46 (m, 2H), 3.43 (d, J=10.8 Hz, 2H), 3.19 (t, J=10.3 Hz, 1H), 2.99 (s, 1H), 2.97-2.73 (m, 3H), 2.65 (d, J=9.3 Hz, 2H), 2.51-2.43 (m, 3H), 2.31 (d, J=11.0 Hz, 1H), 2.18 (s, 1H), 1.95 (t, J=10.4 Hz, 1H), 1.79 (d, J=12.9 Hz, 1H), 1.61 (s, 3H), 1.58 (s, 3H), 0.92 (dd, J=6.2, 3.7 Hz, 6H).

Example 137: 1-(2-(6-oxa-3-azabicyclo[3.1.1]heptan-3-yl)-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 189)

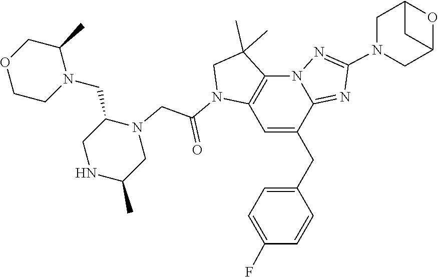 [SMILES: C[C@@H]1CN(CC(=O)N2CC(C)(C)c3c2cc(Cc2ccc(F)cc2)c2nc(N4CC5CC(C4)O5)nn32)[C@@H](CN2CCOC[C@H]2C)CN1]

Following the synthesis of compound 195, compound 189 was obtained as a white solid. MS (ESI) m/z 647.0 [M+H]⁺. ¹H NMR (400 MHz, DMSO-d6) δ (ppm) 8.20 (s, 1H), 7.47-7.39 (m, 2H), 7.19-7.09 (m, 2H), 4.73 (d, J=6.4 Hz, 2H), 4.20 (d, J=3.4 Hz, 2H), 4.13 (d, J=10.6 Hz, 1H), 3.93 (d, J=10.5 Hz, 1H), 3.84 (d, J=9.6 Hz, 1H), 3.80 (d, J=5.2 Hz, 2H), 3.73-3.60 (m, 3H), 3.55-3.45 (m, 2H), 3.18 (q, J=8.3, 7.5 Hz, 2H), 3.01 (s, 1H), 2.97-2.72 (m, 4H), 2.70-2.62 (m, 2H), 2.52-2.45 (m, 2H), 2.38-2.29 (m, 1H), 2.18 (s, 1H), 1.97 (dd, J=19.6, 9.4 Hz, 2H), 1.83-1.75 (m, 1H), 1.64 (s, 3H), 1.59 (s, 3H), 0.93 (dd, J=6.2, 3.5 Hz, 6H).

Example 138: 4-(4-fluorobenzyl)-8,8-dimethyl-6-(2-((2R,5R)-5-methyl-2-(((R)-3-methylmorpholino)methyl)piperazin-1-yl)acetyl)-2-(tetrahydro-2H-pyran-4-yl)-2,6,7,8-tetrahydro-1H-pyrrolo[2,3-e][1,2,4]triazolo[4,3-a]pyridin-1-one (compound 190)

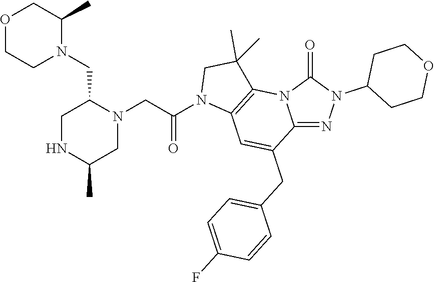 [SMILES: C[C@@H]1CN(CC(=O)N2CC(C)(C)c3c2cc(Cc2ccc(F)cc2)c2nn(C4CCOCC4)c(=O)n32)[C@@H](CN2CCOC[C@H]2C)CN1]

Following the synthesis of compound 135, compound 190 was obtained as a white solid (11.1 mg). ¹H NMR (400 MHz, DMSO-d6) δ (ppm) 7.96 (s, 1H), 7.37 (dd, J=8.3, 5.7 Hz, 2H), 7.14 (t, J=8.9 Hz, 2H), 4.49-4.38 (m, 1H), 4.05-3.90 (m, 6H), 3.76 (d, J=10.7 Hz, 2H), 3.73-3.60 (m, 3H), 3.49-3.48 (m, 2H), 3.17-3.12 (m, 1H), 2.94-2.73 (m, 4H), 2.67 (d, J=1.6 Hz, 2H), 2.41-2.30 (m, 2H), 2.21-2.12 (m, 1H), 2.08-1.89 (m, 3H), 1.84-1.74 (m, 3H), 1.58 (d, J=12.7 Hz, 6H), 0.96 (d, J=6.1 Hz, 3H), 0.89 (d, J=6.2 Hz, 3H). MS (ESI) m/z 650.7 [M+H]⁺.

Example 139: (R)-4-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methyl piperazin-2-yl)methyl)-3-methylpiperazin-2-one (compound 191)

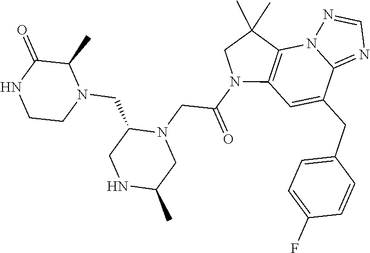 [SMILES: C[C@@H]1CN(CC(=O)N2CC(C)(C)c3c2cc(Cc2ccc(F)cc2)c2ncnn32)[C@@H](CN2CCNC(=O)[C@H]2C)CN1]

Following the synthesis of compound 146, compound 191 was obtained as a white solid (17 mg). ¹H NMR (400 MHz, DMSO-d6) δ 8.50 (s, 1H), 8.39 (s, 1H), 7.50 (s, 11H), 7.38 (dd, J=8.3, 5.7 Hz, 2H), 7.11 (t, J=8.7 Hz, 2H), 4.29 (s, 2H), 4.09 (d, J=10.5 Hz, 1H), 3.92 (d, J=10.5 Hz, 1H), 3.80-3.64 (m, 2H), 3.09 (s, 1H), 2.93 (d, J=8.2 Hz, 2H), 2.80 (dd, J=15.4, 8.9 Hz, 4H), 2.67 (s, 2H), 2.35 (d, J=15.9 Hz, 2H), 2.19 (s, 1H), 2.01 (d, J=10.8 Hz, 11H), 1.56 (d, J=14.0 Hz, 6H), 1.24 (d, J=6.7 Hz, 3H), 0.93 (s, 3H). MS (ESI) m/z 563.5 [M+H]⁺.

Example 140: 4-(4-fluorobenzyl)-8,8-dimethyl-6-(2-((2R,5R)-5-methyl-2-(((R)-3-methylmorpholino)methyl)piperazin-1-yl)acetyl)-2-(pyridin-2-ylmethyl)-2,6,7,8-tetrahydro-1H-pyrrolo[2,3-e][1,2,4]triazolo[4,3-a]pyridin-1-one (compound 192)

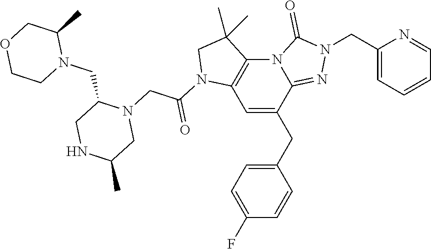 [SMILES: C[C@@H]1CN(CC(=O)N2CC(C)(C)c3c2cc(Cc2ccc(F)cc2)c2nn(Cc4ccccn4)c(=O)n32)[C@@H](CN2CCOC[C@H]2C)CN1]

Following the synthesis of compound 135, compound 192 was obtained as a white solid (13.3 mg). ¹H NMR (400 MHz, DMSO-d⁶) δ (ppm) 8.53 (d, J=4.7 Hz, 1H), 7.96 (s, 1H), 7.83-7.71 (m, 1H), 7.37-7.26 (m, 4H), 7.09 (t, J=8.8 Hz, 2H), 5.22 (s, 2H), 4.02-3.89 (m, 3H), 3.83-3.76 (m, 1H), 3.73-3.62 (m, 2H), 3.54-3.49 (m, 2H), 3.19-3.13 (m, 1H), 3.04 (s, 1H), 2.98-2.89 (m, 1H), 2.87-2.73 (m, 3H), 2.72-2.60 (m, 3H), 2.35-2.30 (m, 1H), 2.23-2.11 (m, 1H), 1.98-1.89 (m, 1H), 1.77 (d, J=11.6 Hz, 1H), 1.59 (d, J=12.0 Hz, 6H), 0.96-0.87 (m, 6H). MS (ESI) m/z 657.6 [M+H]⁺.

Example 141: 4-(4-fluorobenzyl)-8,8-dimethyl-6-(2-((2R,5R)-5-methyl-2-(((R)-3-methylmorpholino)methyl)piperazin-1-yl)acetyl)-2-(pyrazin-2-ylmethyl)-2,6,7,8-tetrahydro-1H-pyrrolo[2,3-e][1,2,4]triazolo[4,3-a]pyridin-1-one (compound 193)

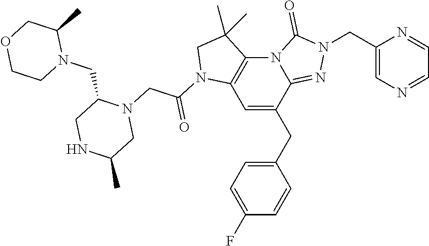 [SMILES: C[C@@H]1CN(CC(=O)N2CC(C)(C)c3c2cc(Cc2ccc(F)cc2)c2nn(Cc4cnccn4)c(=O)n32)[C@@H](CN2CCOC[C@H]2C)CN1]

Following the synthesis of compound 135, compound 193 was obtained as a white solid (27.5 mg). ¹H NMR (400 MHz, DMSO-d⁶) δ (ppm) 8.69 (s, 1H), 8.62 (s, 2H), 7.98 (s, 1H), 7.29 (dd, J=8.5, 5.7 Hz, 2H), 7.08 (t, J=8.8 Hz, 2H), 5.32 (s, 2H), 3.99 (d, J=10.6 Hz, 1H), 3.91 (s, 2H), 3.79 (d, J=10.7 Hz, 1H), 3.72 (d, J=16.7 Hz, 1H), 3.59 (d, J=16.7 Hz, 1H), 3.50 (d, J=10.7 Hz, 2H), 3.19 (t, J=9.9 Hz, 1H), 3.01-2.88 (m, 2H), 2.87-2.75 (m, 2H), 2.75-2.69 (m, 1H), 2.64-2.57 (m, 2H), 2.47-2.41 (m, 1H), 2.32-2.24 (m, 1H), 2.21-2.10 (m, 1H), 1.92 (t, J=9.4 Hz, 1H), 1.74 (d, J=11.8 Hz, 1H), 1.58 (d, J=12.2 Hz, 6H), 0.91-0.85 (m, 6H). MS (ESI) m/z 657.6 [M+H]⁺.

Example 142: 1-(4-(6-fluoro-4-methyl-3,4-dihydroquinoxalin-1(2H)-yl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 194)

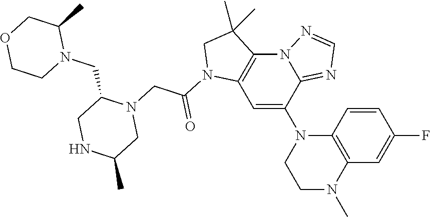 [SMILES: C[C@@H]1CN(CC(=O)N2CC(C)(C)c3c2cc(N2CCN(C)c4cc(F)ccc42)c2ncnn32)[C@@H](CN2CCOC[C@H]2C)CN1]

Following the synthesis of compound 168, compound 194 was obtained as a yellow solid (18 mg).

¹H NMR (400 MHz, DMSO-d⁶) δ (ppm) 8.51 (s, 1H), 8.04 (s, 1H), 6.70 (dd, J=8.7, 6.1 Hz, 1H), 6.63 (dd, J=12.0, 2.7 Hz, 1H), 6.32 (td, J=8.5, 2.8 Hz, 1H), 4.18-4.10 (m, 2H), 4.01-3.91 (m, 2H), 3.80-3.69 (m, 2H), 3.61-3.49 (m, 2H), 3.29-3.17 (m, 4H), 3.13-3.02 (m, 1H), 2.99-2.91 (m, 4H), 2.90-2.54 (m, 6H), 2.38-2.29 (m, 1H), 2.23-2.13 (m, 1H), 2.00-1.90 (m, 1H), 1.84-1.74 (m, 1H), 1.63 (d, J=18.5 Hz, 6H), 1.00-0.88 (m, 6H). MS (ESI) m/z 606.57 [M+H]⁺

Example 143: 4-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)piperazin-2-one (compound 195)

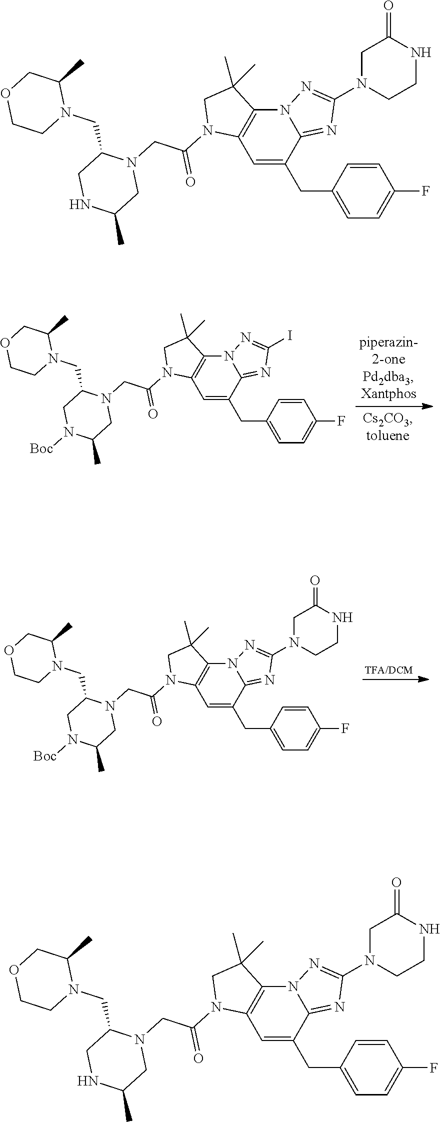 [SMILES: C[C@@H]1CN(CC(=O)N2CC(C)(C)c3c2cc(Cc2ccc(F)cc2)c2nc(N4CCNC(=O)C4)nn32)[C@@H](CN2CCOC[C@H]2C)CN1 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(I)nn21 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(N3CCNC(=O)C3)nn21 ; C[C@@H]1CN(CC(=O)N2CC(C)(C)c3c2cc(Cc2ccc(F)cc2)c2nc(N4CCNC(=O)C4)nn32)[C@@H](CN2CCOC[C@H]2C)CN1]

Step 1: tert-butyl (2R,5S)-4-(2-(4-(4-fluorobenzyl)-8,8-dimethyl-2-(3-oxopiperazin-1-yl)-7,8-dihydro-6H-pyrrolo[2,3-e][1,2,4]triazolo[1,5-a]pyridin-6-yl)-2-oxoethyl)-2-methyl-5-(((R)-3-m ethylmorpholino)methyl)piperazine-1-carboxylate

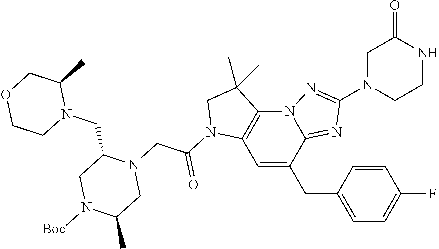 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(N3CCNC(=O)C3)nn21]

A mixture of tert-butyl (2R,5S)-4-(2-(4-(4-fluorobenzyl)-2-iodo-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino) methyl)piperazine-1-carboxylate (100 mg, 0.129 mmol, 1.0 eq), piperazin-2-one (19.36 mg, 0.193 mmol, 1.5 eq), Cs₂CO₃ (84 mg, 0.258 mmol, 2.0 eq), Pd₂dba₃ (11.81 mg, 0.013 mmol, 0.1 eq) and xantphos (7.46 mg, 0.013 mmol, 0.1 eq) in Toluene (10 mL) was stirred at 100° C. for 16 h. the mixture was filtered, the filtrate was concentrated to give a yellow oil, which was purified by column eluted with 70% EA/PE to give the title compound (90 mg, 93%) as a yellow oil. MS (ESI) m/z 749.71 [M+H]⁺

Step 2: 4-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)piperazin-2-one (compound 195)

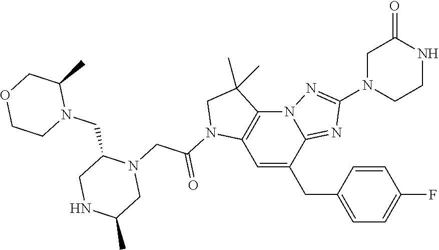 [SMILES: C[C@@H]1CN(CC(=O)N2CC(C)(C)c3c2cc(Cc2ccc(F)cc2)c2nc(N4CCNC(=O)C4)nn32)[C@@H](CN2CCOC[C@H]2C)CN1]

A mixture of tert-butyl (2R,5S)-4-(2-(4-(4-fluorobenzyl)-8,8-dimethyl-2-(3-oxopiperazin-1-yl)-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (90 mg, 0.120 mmol, 1.0 eq) in TFA (3 mL)/DCM (3.00 mL) was stirred at 20-25° C. for 1 h. the mixture was concentrated. the residue was dissolved in DCM, washed with NaHCO₃(aq), dried over Na₂SO₄ and concentrated to give a yellow oil, which was purified by prep-HPLC to give the title compound (30 mg, 38.5%) as a white solid. ¹H NMR (400 MHz, DMSO-d⁶) δ (ppm) 8.23 (s, 1H), 8.08 (s, 1H), 7.41 (dd, J=8.4, 5.7 Hz, 2H), 7.14 (t, J=8.7 Hz, 2H), 4.23-4.02 (m, 5H), 3.97-3.72 (m, 4H), 3.69-3.45 (m, 3H), 3.32-2.58 (m, 11H), 2.51-2.44 (m, 1H), 2.39-2.27 (m, 1H), 2.23-2.12 (m, 1H), 2.01-1.90 (m, 1H), 1.85-1.73 (m, 1H), 1.59 (d, J=18.2 Hz, 6H), 0.98-0.87 (m, 6H). MS (ESI) m/z 648.61 [M+H]⁺

Example 144: 2-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)acetonitrile (compound 196)

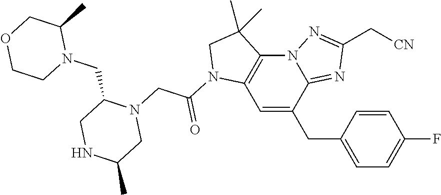 [SMILES: C[C@@H]1CN(CC(=O)N2CC(C)(C)c3c2cc(Cc2ccc(F)cc2)c2nc(CC#N)nn32)[C@@H](CN2CCOC[C@H]2C)CN1]

Following the synthesis of compound 141, compound 196 was obtained as a white solid (23 mg).

¹H NMR (400 MHz, DMSO-d⁶) δ (ppm) 8.38 (s, 1H), 7.40 (dd, J=8.3, 5.7 Hz, 2H), 7.16 (t, J=8.1 Hz, 2H), 4.47 (s, 2H), 4.37-4.26 (m, 2H), 4.17 (d, J=10.5 Hz, 1H), 3.97 (d, J=10.6 Hz, 1H), 3.88-3.80 (m, 1H), 3.74-3.63 (m, 1H), 3.54-3.45 (m, 2H), 3.29-2.56 (m, 10H), 2.41-2.29 (m, 1H), 2.24-2.13 (m, 1H), 2.01-1.91 (m, 1H), 1.85-1.75 (m, 1H), 1.62 (d, J=16.4 Hz, 6H), 0.99-0.87 (m, 6H).

Example 145: 4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxylic acid (compound 197)

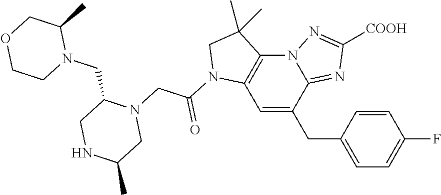 [SMILES: C[C@@H]1CN(CC(=O)N2CC(C)(C)c3c2cc(Cc2ccc(F)cc2)c2nc(C(=O)O)nn32)[C@@H](CN2CCOC[C@H]2C)CN1]

Following the synthesis of compound 31, compound 197 was obtained as a white solid. (30 mg, 36.5%). ¹H NMR (400 MHz, D₂O) δ (ppm) 8.13 (s, 1H), 7.28 (dd, J=8.3, 5.7 Hz, 2H), 6.99 (t, J=8.9 Hz, 2H), 4.35-4.16 (m, 2H), 4.02-3.73 (m, 4H), 3.53-3.40 (m, 3H), 3.34-3.12 (m, 3H), 3.05-2.88 (m, 4H), 2.81-2.69 (m, 2H), 2.33-2.21 (m, 1H), 2.10-1.95 (m, 2H), 1.58 (d, J=19.5 Hz, 6H), 1.21 (d, J=6.5 Hz, 3H), 0.84 (d, J=6.3 Hz, 3H). MS (ESI) m/z 594.51 [M+H]⁺

Example 146: 1-(4-(6-fluoro-3,4-dihydroquinoxalin-1(2H)-yl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 198)

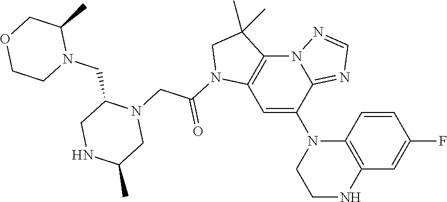 [SMILES: C[C@@H]1CN(CC(=O)N2CC(C)(C)c3c2cc(N2CCNc4cc(F)ccc42)c2ncnn32)[C@@H](CN2CCOC[C@H]2C)CN1]

Following the synthesis of compound 168, compound 198 was obtained as a white solid (30.2 mg). ¹H NMR (400 MHz, DMSO-d⁶) δ (ppm) 8.50 (s, 1H), 8.04 (s, 1H), 6.71 (dd, J=8.7, 5.9 Hz, 1H), 6.47 (dd, J=10.9, 2.8 Hz, 1H), 6.37 (s, 1H), 6.24 (td, J=8.6, 2.8 Hz, 1H), 4.18-4.04 (m, 2H), 3.98-3.86 (m, 2H), 3.82-3.65 (m, 2H), 3.60-3.48 (m, 2H), 3.29-3.15 (m, 4H), 3.07-2.59 (m, 7H), 2.51-2.44 (m, 1H), 2.39-2.25 (m, 1H), 2.23-2.12 (m, 1H), 1.99-1.89 (m, 1H), 1.81-1.73 (m, 1H), 1.62 (d, J=17.9 Hz, 6H), 0.98-0.87 (m, 6H). MS (ESI) m/z 592.52 [M+H]⁺

Example 147: 1-(2-(1,1-dioxidothiomorpholino)-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 199)

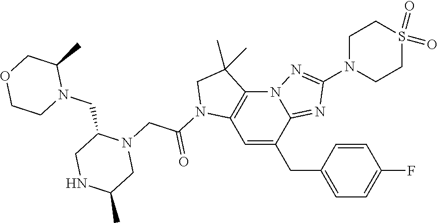 [SMILES: C[C@@H]1CN(CC(=O)N2CC(C)(C)c3c2cc(Cc2ccc(F)cc2)c2nc(N4CCS(=O)(=O)CC4)nn32)[C@@H](CN2CCOC[C@H]2C)CN1]

Following the synthesis of compound 195, compound 199 was obtained as a white solid (26.8 mg). ¹H NMR (400 MHz, DMSO-d⁶) δ (ppm) 8.23 (s, 1H), 7.41 (dd, J=8.5, 5.7 Hz, 2H), 7.14 (t, J=8.9 Hz, 2H), 4.21 (2, J=15.0 Hz, 2H), 4.14-4.03 (m, 5H), 3.96-3.87 (m, 1H), 3.84-3.76 (m, 1H), 3.74-3.63 (m, 1H), 3.54-3.45 (m, 2H), 3.28-3.23 (m, 4H), 3.20-3.13 (m, 1H), 3.10-2.99 (m, 1H), 2.96-2.54 (m, 8H), 2.39-2.28 (m, 1H), 2.24-2.13 (m, 1H), 2.01-1.89 (m, 1H), 1.84-1.75 (m, 1H), 1.59 (d, J=18.3 Hz, 6H), 0.93 (d, J=6.1 Hz, 6H). MS (ESI) m/z 683.56 [M+H]⁺

Example 148: 1-(4-(4-fluorobenzyl)-8,8-dimethyl-2-(methylsulfonyl)-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 200)

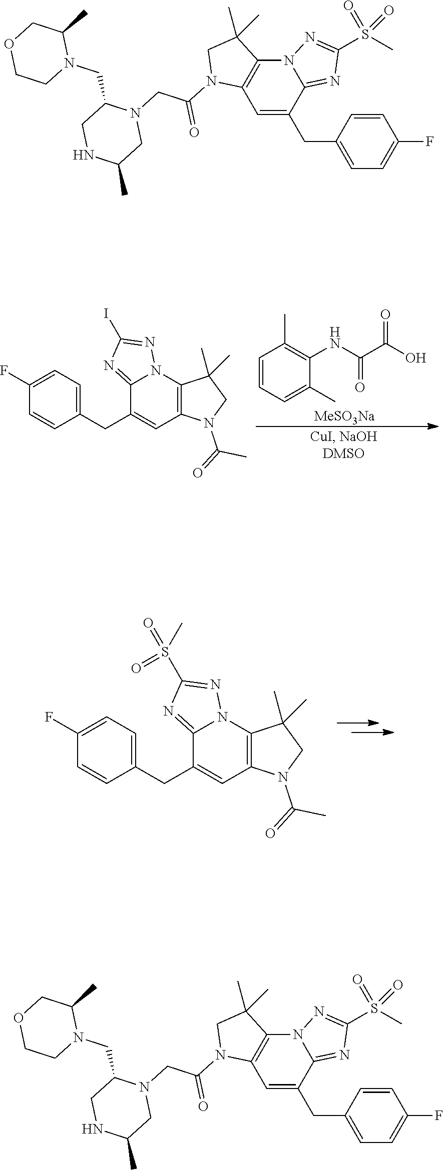

Step 1: 1-(4-(4-fluorobenzyl)-8,8-dimethyl-2-(methylsulfonyl)-7,8-dihydro-6H-pyrrolo[2,3-e][1,2,4]triazolo[1,5-a]pyridin-6-yl)ethan-1-one

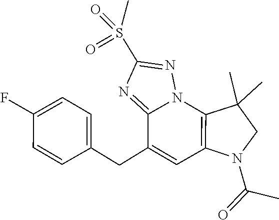 [SMILES: CC(=O)N1CC(C)(C)c2c1cc(Cc1ccc(F)cc1)c1nc(S(C)(=O)=O)nn21]

A mixture of 1-(4-(4-fluorobenzyl)-2-iodo-8,8-dimethyl-7,8-dihydro-6H-pyrrolo[2,3-e][1,2,4]triazolo[1,5-a]pyridin-6-yl)ethan-1-one (130 mg, 0.280 mmol, 1.0 eq), sodium methanesulfinate (286 mg, 2.80 mmol, 10 eq), copper(I) iodide (32.0 mg, 0.168 mmol, 0.6 eq), 2-((2,6-dimethylphenyl)amino)-2-oxoacetic acid (27.0 mg, 0.140 mmol, 0.5 eq) and NaOH (22.40 mg, 0.560 mmol, 2.0 eq) in DMSO (3 mL) was stirred at 120° C. for 2 h in microwave reactor. the mixture was diluted with water, extracted with ethyl acetate (twice). The Organic layer was washed with brine, dried over Na₂SO₄ and concentrated to give the title compound (100 mg, 86%) as a yellow oil. MS (ESI) m/z 417.35 [M+H]⁺

Following steps: compound 200 was obtained (36 mg, 37.9%) as a white solid in a manner similar to that described in compound 14. ¹H NMR (400 MHz, DMSO-d⁶) δ (ppm) 8.53 (s, 1H), 7.48-7.37 (m, 2H), 7.21-7.12 (m, 2H), 4.44-4.31 (m, 2H), 4.26-4.14 (m, 1H), 4.05-3.95 (m, 1H), 3.89-3.62 (m, 2H), 3.54 (s, 3H), 3.52-3.43 (m, 2H), 3.24-2.58 (m, 9H), 2.50-2.44 (m, 1H), 2.37-2.27 (m, 1H), 2.24-2.12 (m, 1H), 1.99-1.90 (m, 1H), 1.86-1.75 (m, 1H), 1.69-1.59 (m, 6H), 0.98-0.87 (m, 6H). MS (ESI) m/z 628.56 [M+H]⁺

Example 149: 4-(4-fluorobenzyl)-2-(2-hydroxyethyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 201)

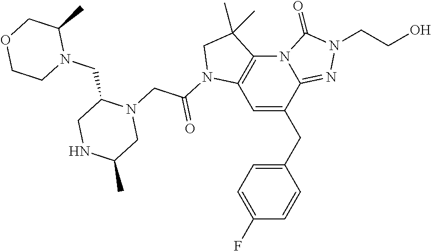 [SMILES: C[C@@H]1CN(CC(=O)N2CC(C)(C)c3c2cc(Cc2ccc(F)cc2)c2nn(CCO)c(=O)n32)[C@@H](CN2CCOC[C@H]2C)CN1]

Following the synthesis of compound 135, compound 201 was obtained as a white solid (13.1 mg). ¹H NMR (400 MHz, DMSO-d6) δ (ppm) 7.96 (s, 1H), 7.35 (dd, J=8.5, 5.7 Hz, 2H), 7.13 (t, J=8.9 Hz, 2H), 4.84 (t, J=5.7 Hz, 1H), 4.00-3.91 (m, 5H), 3.81-3.73 (m, 3H), 3.70-3.59 (m, 2H), 3.51-3.47 (m, 2H), 3.21-3.14 (m, 1H), 3.05-2.98 (m, 1H), 2.96-2.87 (m, 1H), 2.86-2.72 (m, 3H), 2.68-2.57 (m, 3H), 2.35-2.28 (m, 1H), 2.21-2.08 (m, 1H), 1.98-1.85 (m, 1H), 1.75 (d, J=11.9 Hz, 1H), 1.58 (d, J=13.1 Hz, 6H), 0.93-0.87 (m, 6H). MS (ESI) m/z 610.8 [M+H]⁺.

Example 150: 2-((2S,5R)-2-((2-oxa-5-azabicyclo[4.1.0]heptan-5-yl)methy 1)-5-methylpiperazin-1-yl)-1-(4-(4-fluorobenzyl)-8,8-dimethyl-7,8-dihydro-6H-pyrrolo[2,3-e][1,2,4]triazolo[1,5-a]pyridin-6-yl)ethan-1-one (compound 202)

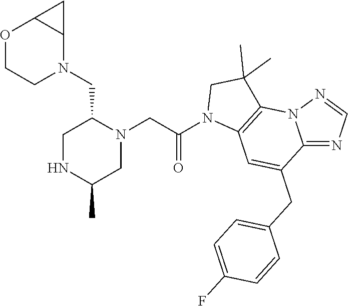 [SMILES: C[C@@H]1CN(CC(=O)N2CC(C)(C)c3c2cc(Cc2ccc(F)cc2)c2ncnn32)[C@@H](CN2CCOC3CC32)CN1]

Following the synthesis of compound 14, compound 202 was obtained as a white solid (20 mg). ¹H NMR (400 MHz, DMSO-d⁶) δ (ppm) 8.52 (s, 1H), 8.37 (s, 1H), 7.43-7.34 (m, 2H), 7.11 (t, J=8.7 Hz, 2H), 4.34-4.21 (m, 2H), 4.18-4.09 (m, 1H), 4.05 (d, J=10.6 Hz, 1H), 3.87 (dd, J=54.0, 13.4 Hz, 1H), 3.66 (s, 1H), 3.49 (d, J=16.3 Hz, 1H), 3.21-3.13 (m, 1H), 3.10 (t, J=10.6 Hz, 1H), 3.05-2.91 (m, 2H), 2.79 (d, J=12.1 Hz, 1H), 2.72-2.58 (m, 4H), 2.41-2.31 (m, 2H), 2.29-2.14 (m, 1H), 2.12-1.92 (m, 1H), 1.62-1.55 (m, 6H), 0.91 (d, J=5.6 Hz, 3H), 0.58-0.42 (m, 1H), 0.27-0.14 (m, 1H). MS (ESI) m/z 548.6 [M+H]⁺.

Example 151: 1-(4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-2-methyl-4-(oxetan-3-yl)piperazin-1-yl)methyl)piperazin-1-yl)ethan-1-one (compound 203)

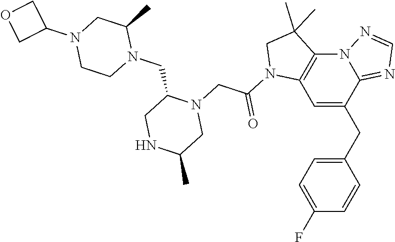 [SMILES: C[C@@H]1CN(CC(=O)N2CC(C)(C)c3c2cc(Cc2ccc(F)cc2)c2ncnn32)[C@@H](CN2CCN(C3COC3)C[C@H]2C)CN1]

Following the synthesis of compound 174, compound 203 was obtained as a white solid (48 mg). ¹H NMR (400 MHz, DMSO-d⁶) δ (ppm) 8.52 (s, 1H), 8.36 (s, 1H), 7.39 (dd, J=8.2, 5.8 Hz, 2H), 7.12 (t, J=8.8 Hz, 2H), 4.36-4.06 (m, 8H), 3.93 (d, J=10.6 Hz, 1H), 3.80 (d, J=16.7 Hz, 1H), 3.68 (d, J=16.7 Hz, 1H), 3.12-3.04 (m, 1H), 3.00-2.93 (m, 1H), 2.92-2.81 (m, 3H), 2.76-2.66 (m, 2H), 2.65-2.54 (m, 1H), 2.43-2.32 (m, 2H), 2.31-2.13 (m, 2H), 1.96 (t, J=9.8 Hz, 1H), 1.80 (d, J=12.9 Hz, 1H), 1.76-1.66 (m, 1H), 1.59 (d, J=16.8 Hz, 6H), 0.96 (dd, J=14.7, 6.1 Hz, 6H). MS (ESI) m/z 605.6 [M+H]⁺.

Example 152: 3-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)oxazolidin-2-one (compound 204)

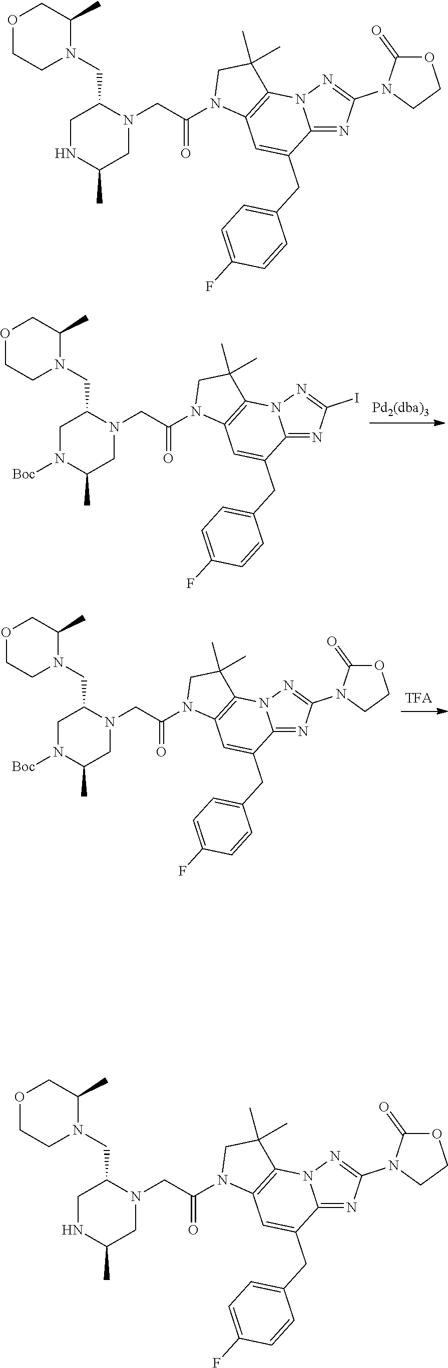 [SMILES: C[C@@H]1CN(CC(=O)N2CC(C)(C)c3c2cc(Cc2ccc(F)cc2)c2nc(N4CCOC4=O)nn32)[C@@H](CN2CCOC[C@H]2C)CN1 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(I)nn21 ; C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(N3CCOC3=O)nn21 ; C[C@@H]1CN(CC(=O)N2CC(C)(C)c3c2cc(Cc2ccc(F)cc2)c2nc(N4CCOC4=O)nn32)[C@@H](CN2CCOC[C@H]2C)CN1]

Step 1: tert-butyl (2R,5S)-4-(2-(4-(4-fluorobenzyl)-8,8-dimethyl-2-(2-oxooxazolidin-3-yl)-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

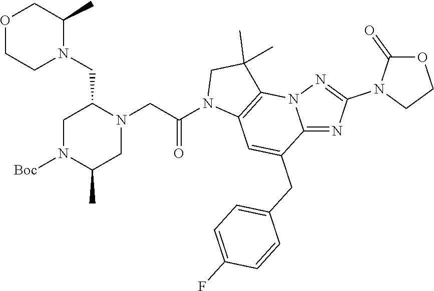 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nc(N3CCOC3=O)nn21]

In a flame-dried 25 mL round-bottomed flask, tert-butyl (2R,5S)-4-(2-(4-(4-fluorobenzyl)-2-iodo-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (80 mg, 0.103 mmol, 1.0 eq), oxazolidin-2-one (17.96 mg, 0.206 mmol, 2.0 eq), Pd₂(dba)₃ (14.17 mg, 0.015 mmol, 0.15 eq), xantphos (17.90 mg, 0.031 mmol, 0.3 eq) and Cs₂CO₃ (67.2 mg, 0.206 mmol, 2.0 eq) were dissolved in dioxane (10 mL) under nitrogen. The reaction was stirred for 14 h at 100 CC. H₂O (20 ml) was added to the reaction mixture followed by extraction with ethyl acetate (10 ml×3). The combined organic layers were dried over Na₂SO₄, filtered and concentrated. The crude product was added to a silica gel column and was eluted with MeOH/DCM from 0-10% to give the title compound (70 mg, 92%) as an off-white solid. MS (ESI) m/z 736.3 [M+H]⁺.

Step 2: compound 204 was obtained as a white solid after lyophilization after acidic deprotection and pre-HPLC purification. MS (ESI) m/z 635.5 [M+H]⁺. ¹H NMR (400 MHz, DMSO-d⁶) δ (ppm) 8.34 (d, J=3.2 Hz, 1H), 7.43 (dd, J=8.4, 5.6 Hz, 2H), 7.15 (t, J=8.8 Hz, 2H), 4.54 (t, J=7.9 Hz, 2H), 4.34-4.20 (m, 4H), 4.17 (d, J=11.0 Hz, 1H), 3.97 (d, J=10.5 Hz, 1H), 3.85 (d, J=16.9 Hz, 1H), 3.67 (d, J=16.2 Hz, 1H), 3.55-3.45 (m, 2H), 3.31-3.13 (m, 2H), 3.03 (s, 2H), 2.96-2.75 (m, 4H), 2.72-2.62 (m, 2H), 2.42-2.27 (m, 1H), 2.19 (s, 1H), 1.96 (t, J=11.2 Hz, 1H), 1.84-1.76 (m, 1H), 1.64 (s, 3H), 1.60 (s, 3H), 0.94 (dd, J=6.6, 3.5 Hz, 6H).

Example 153: (R)-4-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methyl piperazin-2-yl)methyl)-1,3-dimethylpiperazin-2-one (compound 205)

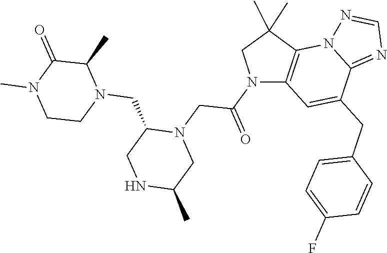 [SMILES: C[C@@H]1CN(CC(=O)N2CC(C)(C)c3c2cc(Cc2ccc(F)cc2)c2ncnn32)[C@@H](CN2CCN(C)C(=O)[C@H]2C)CN1]

Following the synthesis of compound 174, compound 205 was obtained as a white solid (3.1 mg). MS (ESI) m/z 577.6 [M+H]⁺.

Example 154: 1-(2-(1,4-dioxan-2-yl)-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 206)

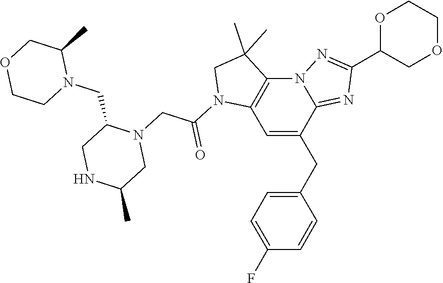 [SMILES: C[C@@H]1CN(CC(=O)N2CC(C)(C)c3c2cc(Cc2ccc(F)cc2)c2nc(C4COCCO4)nn32)[C@@H](CN2CCOC[C@H]2C)CN1]

Following the synthesis of compound 163, compound 206 was obtained as a white solid (15 mg).

NMR (400 MHz, DMSO-d⁶) δ (ppm) 8.36 (s, 1H), 7.41 (dd, J=8.2, 5.8 Hz, 2H), 7.15 (t, J=8.8 Hz, 2H), 4.89 (dd, J=9.5, 2.8 Hz, 1H), 4.37-4.24 (m, 2H), 4.17 (d, J=10.5 Hz, 1H), 4.06-3.64 (m, 9H), 3.56-3.44 (m, 2H), 3.22-3.12 (m, 1H), 3.07-2.61 (m, 8H), 2.47 (d, J=11.0 Hz, 1H), 2.39-2.28 (m, 1H), 2.22-2.11 (m, 1H), 2.02-1.89 (m, 1H), 1.85-1.73 (m, 1H), 1.61 (d, J=17.1 Hz, 6H), 0.99-0.83 (m, 6H). MS (ESI) m/z 636.64 [M+H]⁺

Example 155: 7-fluoro-2-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)isoindolin-1-one (compound 207)

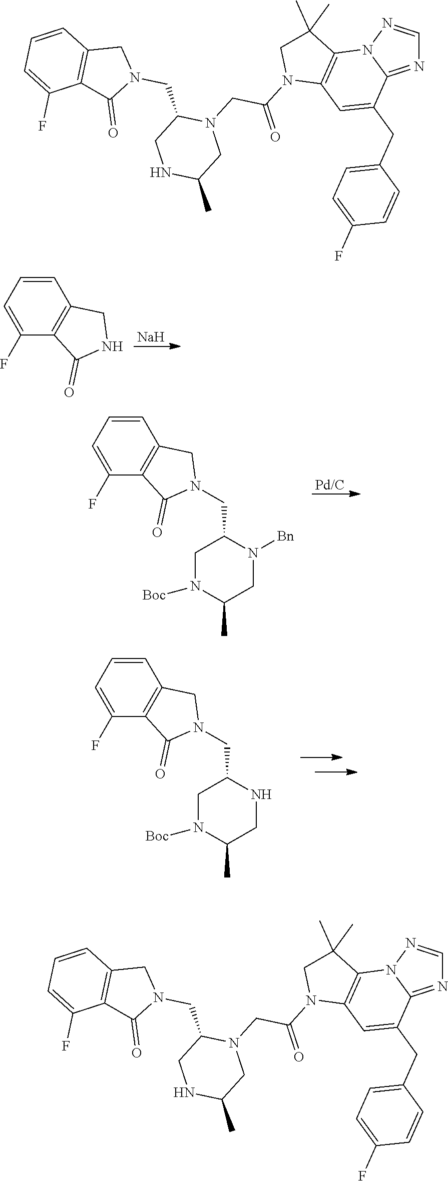

Step 1: tert-butyl (2R,5S)-4-benzyl-5-((7-fluoro-1-oxoisoindolin-2-yl)methyl)-2-methylpiperazine-1-carboxylate

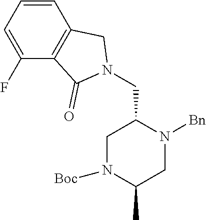 [SMILES: C[C@@H]1CN(Cc2ccccc2)[C@@H](CN2Cc3cccc(F)c3C2=O)CN1C(=O)OC(C)(C)C]

In a flame-dried 100 mL round-bottomed flask, 7-fluoroisoindolin-1-one (147 mg, 0.974 mmol, 1.1) and NaH (31.9 mg, 1.328 mmol, 1.5) were dissolved in DMF (5 mL) under nitrogen. The result mixture was stirred for 0.5 h. KI (162 mg, 0.974 mmol, 1.1 eq) and tert-butyl (2R,5R)-4-benzyl-5-(chloromethyl)-2-methylpiperazine-1-carboxylate (300 mg, 0.885 mmol, 1.0 eq) were added to the reaction mixture in one portion. The reaction mixture was stirred for 3 h at 80° C. The reaction mixture was cooled to 0° C. H₂O (10 mL) was added to the reaction mixture followed by extraction with ethyl acetate (10 mL×3) and washed with brine (10 mL×3). The combined organic layers were dried over Na₂SO₄, filtered and concentrated. The crude product was added to a silica gel column and was eluted with EA/PE from 0-40% to give the title compound (200 mg, 49.8%) as a colorless oil. MS (ESI) m/z 454.8 [M+H]⁺.

Step 2: tert-butyl (2R,5S)-5-((7-fluoro-1-oxoisoindolin-2-yl)methyl)-2-methylpiperazine-1-carboxylate

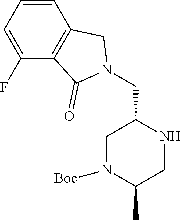 [SMILES: C[C@@H]1CN[C@@H](CN2Cc3cccc(F)c3C2=O)CN1C(=O)OC(C)(C)C]

In a flame-dried 50 mL round-bottomed flask, tert-butyl (2R,5S)-4-benzyl-5-((7-fluoro-1-oxoisoindolin-2-yl)methyl)-2-methylpiperazine-1-carboxylate (200 mg, 0.441 mmol, 1.0 eq) and Pd(OH)2 (61.9 mg, 0.441 mmol, 1.0 eq) were dissolved in IPA (10 mL) under H₂. The reaction mixture was stirred for 14 h and filtered through a celite. The filter layer was concentrated to give the title compound (160 mg, 100%) as a light brown oil. MS (ESI) m/z 364.8 [M+H]⁺.

Following Steps: following the synthesis of compound 14, the desired compound 207 was obtained as a white solid after lyophilization. MS (ESI) m/z 601.3 [M+H]⁺.

Example 156: 4-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)morpholin-3-one (compound 208)

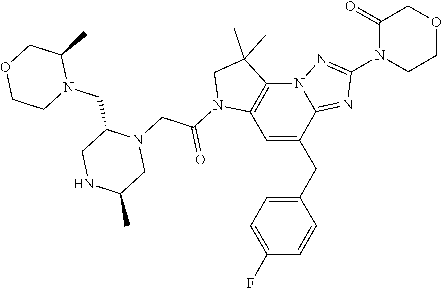 [SMILES: C[C@@H]1CN(CC(=O)N2CC(C)(C)c3c2cc(Cc2ccc(F)cc2)c2nc(N4CCOCC4=O)nn32)[C@@H](CN2CCOC[C@H]2C)CN1]

Following the synthesis of compound 195, the desired compound 208 was obtained as a white solid. MS (ESI) m/z 649.7[M+H]⁺.

Example 157: 1-(2-(dimethylphosphoryl)-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 209)

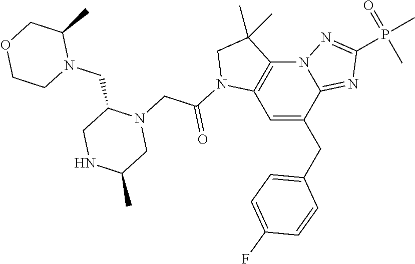 [SMILES: C[C@@H]1CN(CC(=O)N2CC(C)(C)c3c2cc(Cc2ccc(F)cc2)c2nc(P(C)(C)=O)nn32)[C@@H](CN2CCOC[C@H]2C)CN1]

MS (ESI) m/z 626.5[M+H]⁺. ¹H NMR (400 MHz, DMSO-d⁶) δ (ppm) 8.44 (s, 1H), 7.48-7.40 (m, 2H), 7.21-7.11 (m, 2H), 4.43-4.30 (m, 2H), 4.19 (d, J=10.5 Hz, 1H), 3.99 (d, J=10.6 Hz, 1H), 3.84 (d, J=16.8 Hz, 1H), 3.72 (d, J=16.8 Hz, 1H), 3.60-3.45 (m, 2H), 3.15 (t, J=10.3 Hz, 1H), 3.07 (s, 1H), 2.96-2.75 (m, 3H), 2.68 (d, J=10.3 Hz, 2H), 2.58-2.54 (m, 3H), 2.39-2.30 (m, 1H), 2.18 (s, 1H), 1.96 (t, J=9.7 Hz, 1H), 1.92 (s, 3H), 1.88 (s, 3H), 1.80 (d, J=13.3 Hz, 1H), 1.66 (s, 3H), 1.62 (s, 3H), 0.94 (d, J=6.1 Hz, 6H).

Example 158: 4-(4-fluorobenzyl)-8,8-dimethyl-6-(2-((2R,5R)-5-methyl-2-(((R)-3-methylmorpholino)methyl)piperazin-1-yl)acetyl)-2-(oxetan-3-yl)-2,6,7,8-tetrahydro-1H-pyrrolo[2,3-e][1,2,4]triazolo[4,3-a]pyridin-1-one (compound 210)

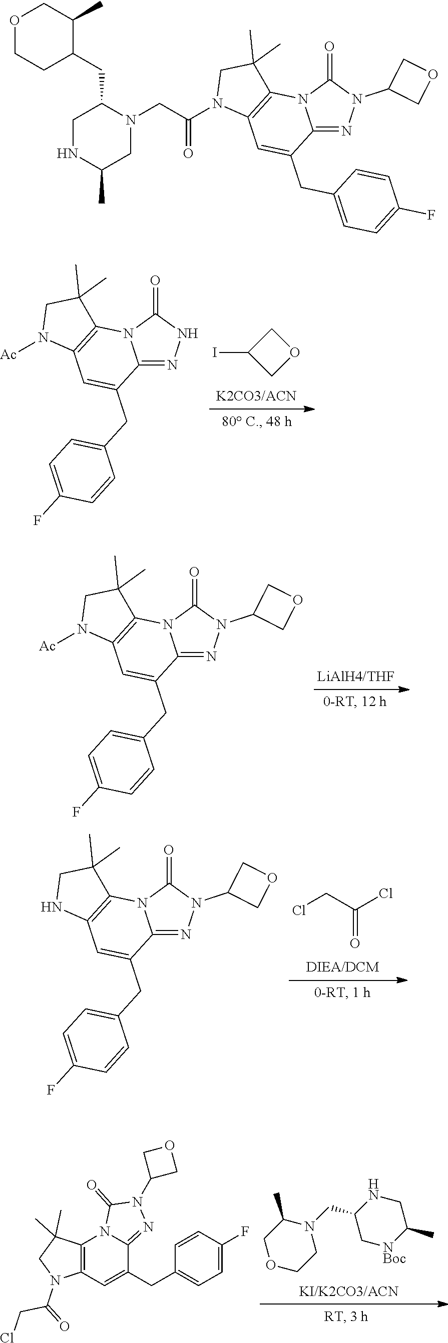

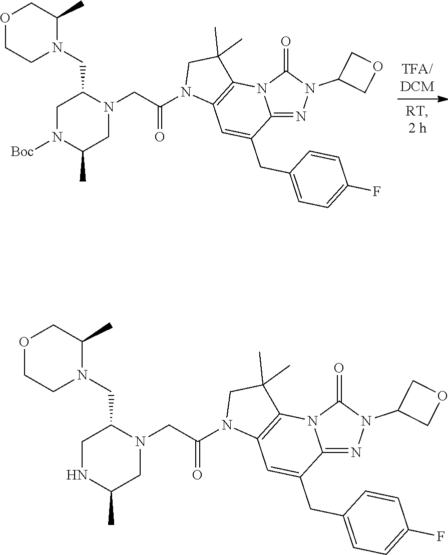 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1)c1nn(C3COC3)c(=O)n21 ; C[C@@H]1CN(CC(=O)N2CC(C)(C)c3c2cc(Cc2ccc(F)cc2)c2nn(C4COC4)c(=O)n32)[C@@H](CN2CCOC[C@H]2C)CN1]

Step 1: 6-acetyl-4-(4-fluorobenzyl)-8,8-dimethyl-2-(oxetan-3-yl)-2,6,7,8-tetrahydro-1H-pyrrolo[2,3-e][1,2,4]triazolo[4,3-a]pyridin-1-one

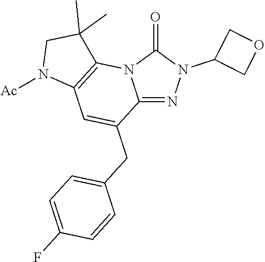 [SMILES: CC(=O)N1CC(C)(C)c2c1cc(Cc1ccc(F)cc1)c1nn(C3COC3)c(=O)n21]

6-Acetyl-4-(4-fluorobenzyl)-8,8-dimethyl-2,6,7,8-tetrahydro-1H-pyrrolo[2,3-e][1,2,4]triazolo[4,3-a]pyridin-1-one (0.2 g, 0.564 mmol, 1.0 eq), 3-iodooxetane (0.519 g, 2.82 mmol, 5.0 eq), and K₂CO₃ (0.234 g, 1.693 mmol, 3.0 eq) were dissolved in acetonitrile (15 mL) under nitrogen to give a yellow suspension. The reaction mixture was stirred at 80° C. for 48 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 100% to give the product (0.138 g, 59.6%) as a yellow oil. MS (ESI) m/z 411.5 [M+H]⁺.

Step 2: 4-(4-fluorobenzyl)-8,8-dimethyl-2-(oxetan-3-yl)-2,6,7,8-tetrahydro-1H-pyrrolo[2,3-e][1,2,4]triazolo[4,3-a]pyridin-1-one

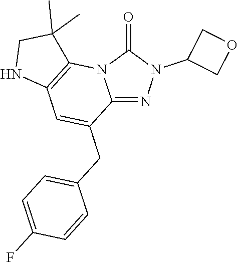 [SMILES: CC1(C)CNc2cc(Cc3ccc(F)cc3)c3nn(C4COC4)c(=O)n3c21]

6-Acetyl-4-(4-fluorobenzyl)-8,8-dimethyl-2-(oxetan-3-yl)-2,6,7,8-tetrahydro-1H-pyrrolo[2,3-e][1,2,4]triazolo[4,3-a]pyridin-1-one (0.138 g, 0.336 mmol, 1.0 eq) and LiAlH₄ (0.026 g, 0.672 mmol, 2.0 eq) were dissolved in THF (15 mL) under argon to give a yellow solution. The reaction mixture was stirred at rt for 2 h. H₂O (1 mL) was added to the reaction mixture followed by extraction with ethyl acetate (30 mL×2). The combined organic layers were dried over Na₂SO₄, filtered and concentrated to give the product (0.124 g, 100%). MS (ESI) m/z 369.4 [M+H]⁺.

Step 3-5: Following the synthesis of compound 14, steps 3-5 were performed to give the title compound as a white solid (10.4 mg). ¹H NMR (400 MHz, DMSO-d⁶) δ (ppm) 8.00 (s, 1H), 7.40 (dd, J=8.4, 5.7 Hz, 2H), 7.15 (t, J=8.9 Hz, 2H), 5.55 (t, J=7.1 Hz, 1H), 4.95-4.86 (m, 4H), 4.03 (d, J=3.1 Hz, 2H), 3.95 (d, J=10.6 Hz, 1H), 3.77 (d, J=10.6 Hz, 1H), 3.67 (s, 2H), 3.50-3.46 (m, 2H), 3.15-3.10 (m, 1H), 2.88-2.73 (m, 4H), 2.67-2.62 (m, 2H), 2.41-2.30 (m, 2H), 2.22-2.10 (m, 1H), 1.99-1.87 (m, 2H), 1.78 (d, J=12.2 Hz, 1H), 1.55 (d, J=12.7 Hz, 6H), 0.92 (dd, J=23.3, 6.2 Hz, 6H). MS (ESI) m/z 622.5 [M+H]⁺.

Example 159: 1-(4-((5-fluoropyridin-2-yl)thio)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 211)

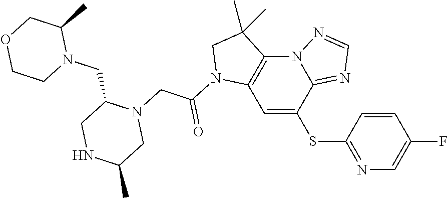 [SMILES: C[C@@H]1CN(CC(=O)N2CC(C)(C)c3c2cc(Sc2ccc(F)cn2)c2ncnn32)[C@@H](CN2CCOC[C@H]2C)CN1]

Following the synthesis of compound 168, compound 211 was obtained as a white solid (52 mg).

NMR (400 MHz, DMSO-d⁶) δ (ppm) 8.73 (s, 1H), 8.54 (s, 1H), 8.47 (d, J=2.9 Hz, 1H), 7.71 (td, J=8.7, 3.0 Hz, 1H), 7.37 (dd, J=8.8, 4.1 Hz, 1H), 4.31-4.22 (m, 1H), 4.11-4.01 (m, 1H), 3.98-3.87 (m, 1H), 3.72-3.51 (m, 3H), 3.32-3.21 (m, 2H), 3.05-2.68 (m, 8H), 2.44-2.31 (m, 1H), 2.27-2.16 (m, 1H), 2.05-1.95 (m, 1H), 1.88-1.78 (m, 1H), 1.68 (d, J=13.0 Hz, 6H), 1.00-0.91 (m, 6H). MS (ESI) m/z 569.51 [M+H]⁺

Example 160: 1-(2-((dimethylamino)methyl)-4-(4-fluorobenzyl)-8,8-dim ethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 212)

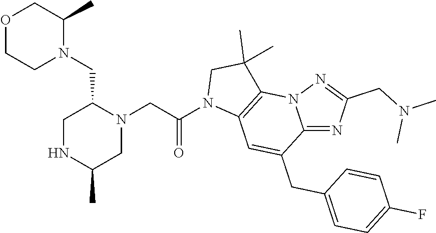 [SMILES: C[C@@H]1CN(CC(=O)N2CC(C)(C)c3c2cc(Cc2ccc(F)cc2)c2nc(CN(C)C)nn32)[C@@H](CN2CCOC[C@H]2C)CN1]

Following the synthesis of compound 141, compound 212 was obtained as a white solid (15 mg).

¹H NMR (400 MHz, DMSO-d⁶) δ (ppm) 8.33 (s, 1H), 7.41 (dd, J=8.3, 5.7 Hz, 2H), 7.15 (t, J=8.9 Hz, 2H), 4.35-4.24 (m, 2H), 4.17 (d, J=10.6 Hz, 1H), 3.96 (d, J=10.6 Hz, 1H), 3.84 (d, J=16.6 Hz, 1H), 3.74-3.64 (m, 3H), 3.53-3.46 (m, 2H), 3.21-3.13 (m, 1H), 3.06-2.97 (m, 1H), 2.95-2.55 (m, 7H), 2.51-2.45 (m, 1H), 2.38-2.27 (m, 7H), 2.23-2.11 (m, 1H), 2.00-1.91 (m, 1H), 1.83-1.74 (m, 1H), 1.61 (d, J=16.9 Hz, 6H), 0.99-0.86 (m, 6H). MS (ESI) m/z 607.62 [M+H]⁺

Example 161: 2-(5-(((2S,5R)-1-(2-(4-(4-fluorobenzyl)-8,8-dimethyl-7,8-di hydro-6H-pyrrolo[2,3-e][1,2,4]triazolo[1,5-a]pyridin-6-yl)-2-oxoethyl)-5-methylpiperazin-2-yl)methyl)-2,5-diazabicyclo[4.1.0]heptan-2-yl)acetonitrile (compound 213)

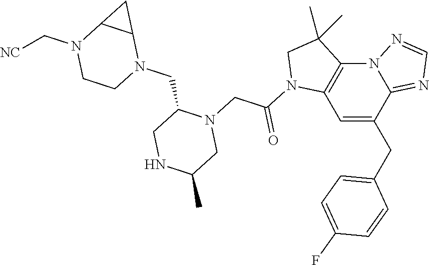 [SMILES: C[C@@H]1CN(CC(=O)N2CC(C)(C)c3c2cc(Cc2ccc(F)cc2)c2ncnn32)[C@@H](CN2CCN(CC#N)C3CC32)CN1]

Following the synthesis of compound 174, compound 213 was obtained as a white solid (5 mg). MS (ESI) m/z 586.4 [M+H]⁺.

Example 162: 2-((2R,5R)-2-((2-oxa-5-azabicyclo[2.2.2]octan-5-yl)methyl)-5-methylpiperazin-1-yl)-1-(4-(4-fluorobenzyl)-8,8-dimethyl-7,8-dihydro-6H-pyrrolo[2,3-e][1,2,4]triazolo[1,5-a]pyridin-6-yl)ethan-1-one (compound 214)

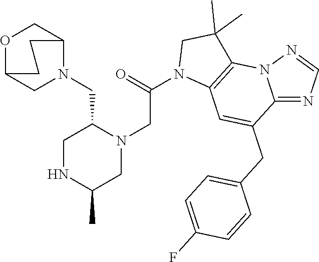 [SMILES: C[C@@H]1CN(CC(=O)N2CC(C)(C)c3c2cc(Cc2ccc(F)cc2)c2ncnn32)[C@@H](CN2CC3CCC2CO3)CN1]

Following the synthesis of compound 174, compound 214 was obtained as a white solid (58 mg). ¹H NMR (400 MHz, DMSO-d⁶) δ (ppm) 8.51 (s, 1H), 8.42 (s, 1H), 7.39 (dd, J=8.3, 5.7 Hz, 2H), 7.10 (t, J=8.8 Hz, 2H), 4.29 (s, 2H), 4.17 (d, J=10.6 Hz, 1H), 4.01 (d, J=10.6 Hz, 1H), 3.80 (d, J=16.5 Hz, 1H), 3.62 (d, J=16.5 Hz, 1H), 3.30-3.28 (m, 1H), 3.27-3.21 (m, 2H), 2.96 (dd, J=15.9, 4.5 Hz, 2H), 2.86-2.71 (m, 2H), 2.65-2.55 (m, 2H), 2.43-2.20 (m, 3H), 2.02 (dd, J=13.0, 3.0 Hz, 1H), 1.88-1.71 (m, 2H), 1.59 (t, J=8.2 Hz, 9H), 0.88 (d, J=6.0 Hz, 3H). MS (ESI) m/z 562.5 [M+H]⁺.

Example 163: (R)-4-(((2R,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methyl piperazin-2-yl)methyl)-3-methylpiperazine-1-carboxamide (compound 215)

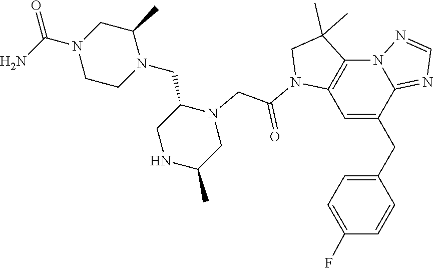 [SMILES: C[C@@H]1CN(CC(=O)N2CC(C)(C)c3c2cc(Cc2ccc(F)cc2)c2ncnn32)[C@@H](CN2CCN(C(N)=O)C[C@H]2C)CN1]

Following the synthesis of compound 174, compound 215 was obtained as a white solid (55 mg). ¹H NMR (400 MHz, DMSO-d⁶) δ (ppm) 8.49 (s, 1H), 8.41 (s, 1H), 7.38 (dd, J=8.4, 5.7 Hz, 2H), 7.12 (t, J=8.8 Hz, 2H), 5.85 (s, 2H), 4.29 (s, 2H), 4.16 (d, J=10.6 Hz, 1H), 3.97 (d, J=10.6 Hz, 1H), 3.85 (d, J=16.5 Hz, 1H), 3.59 (d, J=16.4 Hz, 1H), 3.47 (d, J=12.4 Hz, 2H), 2.95-2.80 (m, 3H), 2.74 (d, J=9.3 Hz, 2H), 2.66-2.53 (m, 3H), 2.43-2.34 (m, 1H), 2.28 (t, J=10.8 Hz, 1H), 2.17-2.08 (m, 1H), 1.90-1.82 (m, 1H), 1.78 (d, J=11.6 Hz, 1H), 1.58 (d, J=7.6 Hz, 6H), 0.96 (d, J=6.1 Hz, 3H), 0.87 (d, J=6.0 Hz, 3H). MS (ESI) m/z 592.6 [M+H]⁺.

Example 164: 1-(4-((2-amino-4-fluorophenyl)thio)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 216)

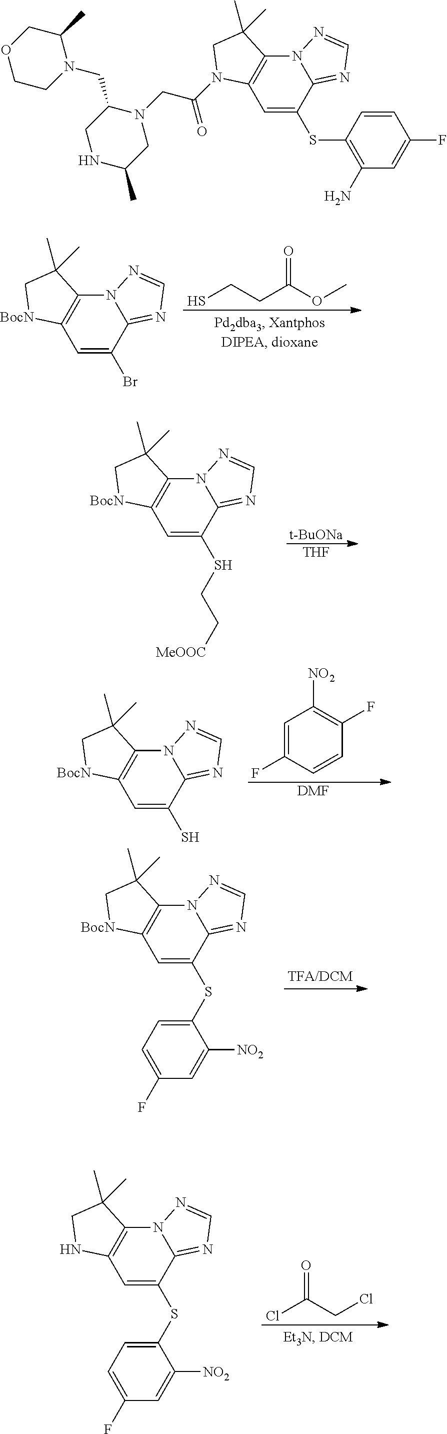

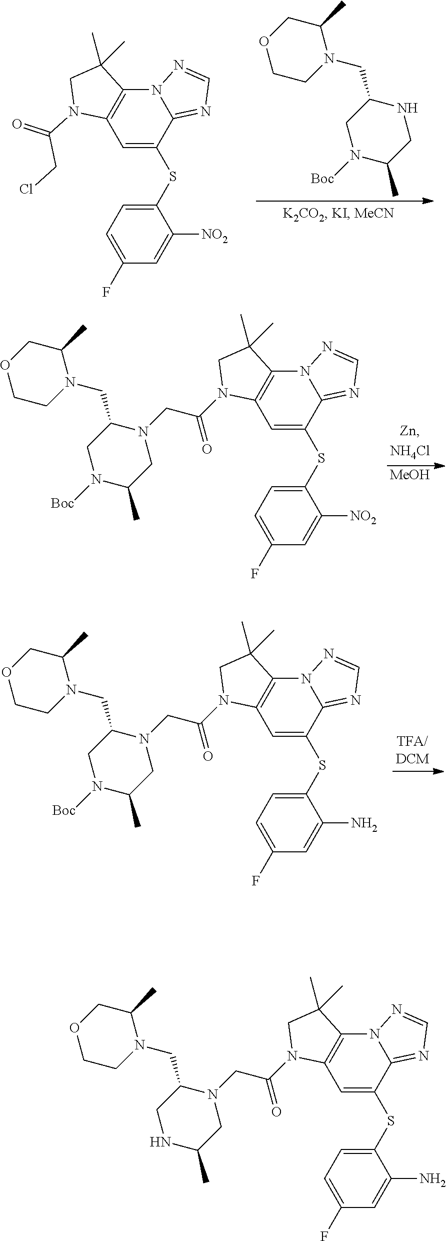

Step 1: tert-butyl 4-((3-methoxy-3-oxopropyl)-l3-sulfanyl)-8,8-dimethyl-7,8-dihydro-6H-pyrrolo[2,3-e][1,2,4]triazolo[1,5-a]pyridine-6-carboxylate

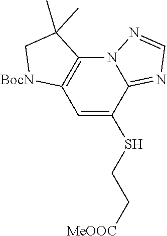 [SMILES: COC(=O)CCSc1cc2c(n3ncnc13)C(C)(C)CN2C(=O)OC(C)(C)C]

A mixture of tert-butyl 4-bromo-8,8-dimethyl-7,8-dihydro-6H-pyrrolo[2,3-e][1,2,4]triazolo[1,5-a]pyridine-6-carboxylate (300 mg, 0.817 mmol, 1.0 eq), methyl 3-mercaptopropanoate (118 mg, 0.980 mmol, 1.2 eq), DIPEA (317 mg, 2.451 mmol, 3.0 eq), Pd₂dba₃ (74.8 mg, 0.082 mmol, 0.1 eq) and xantphos (47.3 mg, 0.082 mmol, 0.1 eq) in Dioxane (10 mL) was stirred at 100° C. for 6 h. The mixture was concentrated to give a yellow oil, which was purified by column eluted with 3% MeOH/DCM to give the title compound (300 mg, 90%) as a yellow oil. MS (ESI) m/z 407.75 [M+H]⁺

Step 2: tert-butyl 4-mercapto-8,8-dimethyl-7,8-dihydro-6H-pyrrolo[2,3-e][1,2,4]triazolo[1,5-a]pyridine-6-carboxylate

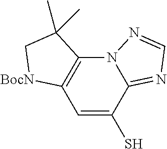 [SMILES: CC(C)(C)OC(=O)N1CC(C)(C)c2c1cc(S)c1ncnn21]

To a solution of tert-butyl 4-((3-methoxy-3-oxopropyl)thio)-8,8-dimethyl-7,8-dihydro-6H-pyrrolo[2,3-e][1,2,4]triazolo[1,5-a]pyridine-6-carboxylate (300 mg, 0.738 mmol, 1.0 eq) in THE (15 mL) was added sodium tert-butoxide (213 mg, 2.214 mmol, 3.0 eq) at 20-25° C. The mixture was stirred for 2 h, then concentrated to give the title compound as a yellow oil, which was used directly in next step. MS (ESI) m/z 321.35 [M+H]⁺

Step 3: tert-butyl 4-((4-fluoro-2-nitrophenyl)thio)-8,8-dimethyl-7,8-dihydro-6H-pyrrolo[2,3-e][1,2,4]triazolo[1,5-a]pyridine-6-carboxylate

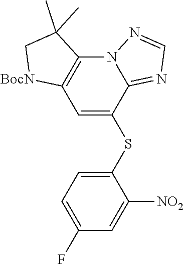 [SMILES: CC(C)(C)OC(=O)N1CC(C)(C)c2c1cc(Sc1ccc(F)cc1[N+](=O)[O-])c1ncnn21]

A mixture of tert-butyl 4-mercapto-8,8-dimethyl-7,8-dihydro-6H-pyrrolo[2,3-e][1,2,4]triazolo[1,5-a]pyridine-6-carboxylate (200 mg, 0.624 mmol, 1.0 eq, theoretical) and 1,4-difluoro-2-nitrobenzene (199 mg, 1.248 mmol, 2.0 eq) in DMF (10 mL) was stirred at rt for 1 h. the mixture was diluted with water, extracted with EA (3 times). The organic layer was washed with brine, dried over Na₂SO₄ and concentrated to give a yellow oil, which was purified by column eluted with 30% EA/PE to give the title compound (220 mg, 77% for 2 steps) as a yellow solid. MS (ESI) m/z 460.76 [M+H]⁺

Step 4: 4-((4-fluoro-2-nitrophenyl)thio)-8,8-dimethyl-7,8-dihydro-6H-pyrrolo[2,3-e][1,2,4]triazolo[1,5-a]pyridine

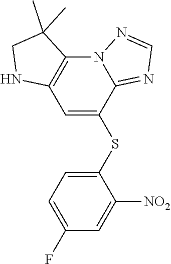 [SMILES: CC1(C)CNc2cc(Sc3ccc(F)cc3[N+](=O)[O-])c3ncnn3c21]

A mixture of tert-butyl 4-((4-fluoro-2-nitrophenyl)thio)-8,8-dimethyl-7,8-dihydro-6H-pyrrolo[2,3-e][1,2,4]triazolo[1,5-a]pyridine-6-carboxylate (220 mg, 0.479 mmol, 1.0 eq) in DCM (5 mL)/TFA (5.00 mL) was stirred at 20-25° C. for 1 h. The mixture was concentrated. The residue was dissolved with DCM, washed with NaHCO₃, brine, dried over Na₂SO₄ and concentrated to give the title compound (170 mg, 99%) as a yellow solid. MS (ESI) m/z 360.67 [M+H]⁺

Step 5: 2-chloro-1-(4-((4-fluoro-2-nitrophenyl)thio)-8,8-dimethyl-7,8-dihydro-6H-pyrrolo[2,3-e][1,2,4]triazolo[1,5-a]pyridin-6-yl)ethan-1-one

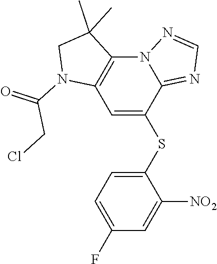 [SMILES: CC1(C)CN(C(=O)CCl)c2cc(Sc3ccc(F)cc3[N+](=O)[O-])c3ncnn3c21]

To a mixture of 4-((4-fluoro-2-nitrophenyl)thio)-8,8-dimethyl-7,8-dihydro-6H-pyrrolo[2,3-e][1,2,4]triazolo[1,5-a]pyridine (170 mg, 0.473 mmol, 1.0 eq) and Et₃N (96 mg, 0.946 mmol, 2.0 eq) in DCM (10 mL) was added 2-chloroacetyl chloride (107 mg, 0.946 mmol, 2.0 eq) at 0-10° C. The reaction mixture was stirred for 1 h. The mixture was quenched with H₂O, extracted with DCM. the organic layer was washed with brine, dried over Na₂SO₄ and concentrated to give the title compound (200 mg, 97%) as a yellow solid. MS (ESI) m/z 436.27 [M+H]⁺

Step 6: tert-butyl (2R,5S)-4-(2-(4-((4-fluoro-2-nitrophenyl)-l3-sulfaneyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

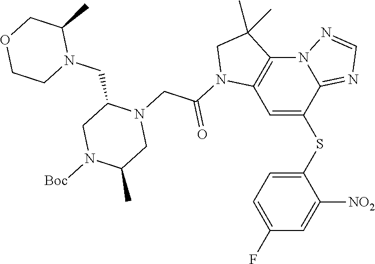 [SMILES: C[C@@H]1COCCN1C[C@H]1CN(C(=O)OC(C)(C)C)[C@H](C)CN1CC(=O)N1CC(C)(C)c2c1cc(Sc1ccc(F)cc1[N+](=O)[O-])c1ncnn21]

A mixture of 2-chloro-1-(4-((4-fluoro-2-nitrophenyl)thio)-8,8-dimethyl-7,8-dihydro-6H-pyrrolo[2,3-e][1,2,4]triazolo[1,5-a]pyridin-6-yl)ethan-1-one (200 mg, 0.459 mmol, 1.0 eq), tert-butyl (2R,5S)-2-methyl-5-(((R)-3-methylmorpholino)methyl)piperazine-1-carboxylate (144 mg, 0.459 mmol, 1.0 eq), K₂CO₃ (127 mg, 0.918 mmol, 2.0 eq) and KI (114 mg, 0.688 mmol, 1.5 eq) in Acetonitrile (20 mL) was stirred at 20-25° C. for 16 h. the mixture was diluted with water, extracted with EA (twice). the organic layer was washed with brine, dried over Na₂SO₄ and concentrated to give a yellow oil, which was purified by column eluted with 3% MeOH/DCM to give the title compound (300 mg, 92%) as a yellow oil. MS (ESI) m/z 713.60 [M+H]

Step 7: tert-butyl (2R,5S)-4-(2-(4-((2-amino-4-fluorophenyl)-l3-sulfaneyl)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate

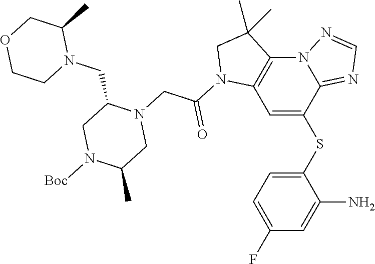 [SMILES: C[C@@H]1COCCN1C[C@H]1CN(C(=O)OC(C)(C)C)[C@H](C)CN1CC(=O)N1CC(C)(C)c2c1cc(Sc1ccc(F)cc1N)c1ncnn21]

A mixture of tert-butyl (2R,5S)-4-(2-(4-((4-fluoro-2-nitrophenyl)thio)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (300 mg, 0.421 mmol, 1.0 eq), ammonium chloride (113 mg, 2.104 mmol, 5.0 eq) and zinc (138 mg, 2.104 mmol, 5.0 eq) in MeOH (20 mL) was stirred at 60° C. for 8 h. the mixture was filtered and the filtrate was concentrated. the residue was dissolved with DCM, washed with water, brine, dried over Na₂SO₄ and concentrated to give a yellow oil, which was purified by column eluted with 5% MeOH/DCM to give the title compound (150 mg, 52.2%) as a yellow oil. MS (ESI) m/z 683.52 [M+H]⁺

Step 8: 1-(4-((2-amino-4-fluorophenyl)-l3-sulfaneyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 216)

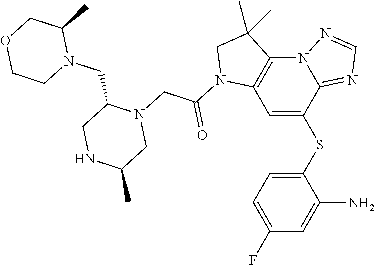 [SMILES: C[C@@H]1CN(CC(=O)N2CC(C)(C)c3c2cc(Sc2ccc(F)cc2N)c2ncnn32)[C@@H](CN2CCOC[C@H]2C)CN1]

A mixture of tert-butyl (2R,5S)-4-(2-(4-((2-amino-4-fluorophenyl)thio)-8,8-dimethyl-7,8-dihydro-6H-pyrrolo[2,3-e][1,2,4]triazolo[1,5-a]pyridin-6-yl)-2-oxoethyl)-2-methyl-5-(((R)-3-methylmorpholino)methyl)piperazine-1-carboxylate (150 mg, 0.220 mmol, 1.0 eq) in TFA (3 mL)/DCM (3.00 mL) was stirred at 20-25° C. for 1 h. the mixture was concentrated. the residue was dissolved in DCM, washed with NaHCO₃(aq), dried over Na₂SO₄ and concentrated to give a yellow oil, which was purified by prep-HPLC to give the title (17 mg, 13.28%) as a white solid.

¹H NMR (400 MHz, DMSO-d⁶) δ (ppm) 8.57 (s, 1H), 7.88 (s, 1H), 7.44 (t, J=7.4 Hz, 1H), 6.65 (d, J=10.0 Hz, 1H), 6.48 (t, J=7.3 Hz, 1H), 5.86 (s, 2H), 4.19 (d, J=10.3 Hz, 1H), 3.94 (d, J=10.5 Hz, 1H), 3.82 (d, J=16.4 Hz, 1H), 3.56-3.48 (m, 2H), 3.24-3.13 (m, 2H), 3.00-2.56 (m, 8H), 2.41-2.25 (m, 2H), 2.17 (s, 1H), 1.99-1.87 (m, 1H), 1.82-1.72 (m, 1H), 1.60 (d, J=20.2 Hz, 6H), 0.99-0.81 (m, 6H). MS (ESI) m/z 583.47 [M+H]⁺

Example 165: 2-(4-(4-fluorobenzyl)-8,8-dimethyl-6-(2-((2R,5R)-5-methyl-2-(((R)-3-methylmorpholino)methyl)piperazin-1-yl)acetyl)-1-oxo-7,8-dihydro-1H-pyrrolo[2,3-e][1,2,4]triazolo[4,3-a]pyridin-2(6H)-yl)acetamide (compound 217)

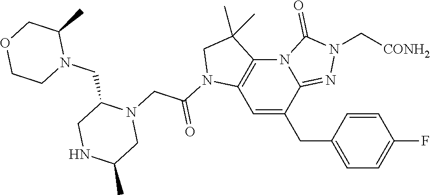 [SMILES: C[C@@H]1CN(CC(=O)N2CC(C)(C)c3c2cc(Cc2ccc(F)cc2)c2nn(CC(N)=O)c(=O)n32)[C@@H](CN2CCOC[C@H]2C)CN1]

Following the synthesis of compound 185, compound 217 was obtained (11 mg, 12.77%) as a white solid. ¹H NMR (400 MHz, DMSO-d⁶) δ (ppm) 7.99 (s, 1H), 7.60 (s, 1H), 7.41-7.32 (m, 3H), 7.16 (t, J=8.8 Hz, 2H), 4.52 (s, 2H), 4.07-3.93 (m, 3H), 3.85-3.48 (m, 5H), 3.26-3.17 (m, 1H), 3.02-2.55 (m, 7H), 2.52-2.44 (m, 2H), 2.36-2.25 (m, 1H), 2.23-2.12 (m, 1H), 1.99-1.87 (m, 1H), 1.82-1.72 (m, 1H), 1.60 (d, J=12.9 Hz, 6H), 0.95-0.86 (m, 6H).

MS (ESI) m/z 623.53 [M+H]⁺

Example 166: 4-(4-fluorobenzyl)-8,8-dimethyl-6-(2-((2R,5R)-5-methyl-2-(((R)-3-methyl morpholino)methyl)piperazin-1-yl)acetyl)-2-((S)-tetrahydrofuran-3-yl)-2,6,7,8-tetrahydro-1H-pyrrolo[2,3-e][1,2,4]triazolo[4,3-a]pyridin-1-one (compound 218)

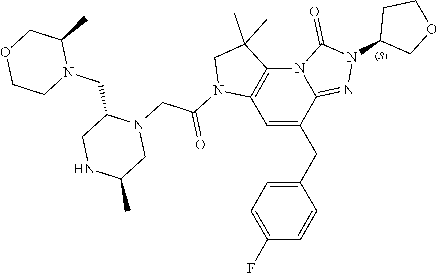

Following the synthesis of compound 228, compound 218 was obtained as a white solid (71.6 mg). MS (ESI) m/z 636.6[M+H]⁺. ¹H NMR (400 MHz, DMSO-d6) δ (ppm) 8.00 (s, 1H), 7.41-7.30 (m, 2H), 7.18-7.07 (m, 2H), 5.06-4.94 (m, 1H), 4.06-3.93 (m, 5H), 3.90-3.81 (m, 2H), 3.77 (d, J=10.7 Hz, 1H), 3.67 (s, 2H), 3.48 (d, J=10.5 Hz, 2H), 3.21-3.03 (m, 2H), 2.93-2.74 (m, 4H), 2.73-2.55 (m, 3H), 2.39-2.20 (m, 3H), 2.20-2.09 (m, 1H), 1.99-1.87 (m, 1H), 1.76 (d, J=12.2 Hz, 1H), 1.57 (d, J=14.3 Hz, 6H), 0.94-0.86 (m, 6H).

Example 167: 4-(4-fluorobenzyl)-2-((6-fluoropyridin-2-yl)methyl)-8,8-di methyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 219)

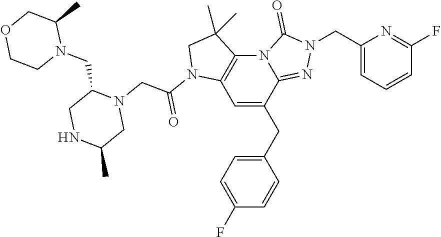 [SMILES: C[C@@H]1CN(CC(=O)N2CC(C)(C)c3c2cc(Cc2ccc(F)cc2)c2nn(Cc4cccc(F)n4)c(=O)n32)[C@@H](CN2CCOC[C@H]2C)CN1]

Following the synthesis of compound 185, compound 219 was obtained as a white solid (31.5 mg). H NMR (400 MHz, DMSO-d⁶) δ (ppm) 8.04-7.95 (m, 2H), 7.37-7.24 (m, 3H), 7.16-7.03 (m, 3H), 5.19 (s, 2H), 4.03-3.88 (m, 3H), 3.79 (d, J=10.6 Hz, 1H), 3.69 (t, J=17.4 Hz, 2H), 3.50 (d, J=10.2 Hz, 2H), 3.21-3.17 (m, 1H), 3.04 (s, 1H), 2.94-2.76 (m, 4H), 2.69-2.60 (m, 2H), 2.37-2.26 (m, 2H), 2.20-2.11 (m, 1H), 1.97-1.89 (m, 1H), 1.77 (d, J=11.6 Hz, 1H), 1.58 (d, J=12.1 Hz, 6H), 0.90 (dt, J=15.0, 7.5 Hz, 6H). MS (ESI) m/z 675.5 [M+H]⁺.

Example 168: 4-(4-fluorobenzyl)-2-((3-fluoropyridin-2-yl)methyl)-8,8-di methyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 220)

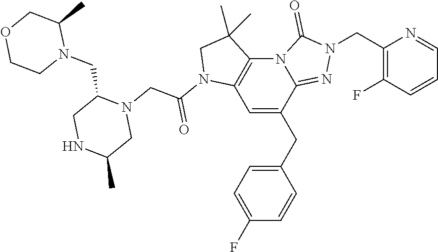 [SMILES: C[C@@H]1CN(CC(=O)N2CC(C)(C)c3c2cc(Cc2ccc(F)cc2)c2nn(Cc4ncccc4F)c(=O)n32)[C@@H](CN2CCOC[C@H]2C)CN1]

Following the synthesis of compound 185, compound 220 was obtained as a white solid (44.3 mg). ¹H NMR (400 MHz, DMSO-d⁶) δ (ppm) 8.38 (d, J=4.3 Hz, 1H), 7.97 (s, 1H), 7.78 (t, J=9.3 Hz, 1H), 7.48 (dt, J=8.4, 4.3 Hz, 1H), 7.33-7.22 (m, 2H), 7.06 (t, J=8.7 Hz, 2H), 5.31 (s, 2H), 3.98 (d, J=10.6 Hz, 1H), 3.88 (s, 2H), 3.78 (d, J=10.6 Hz, 1H), 3.71 (d, J=16.7 Hz, 1H), 3.59 (d, J=16.5 Hz, 1H), 3.51-3.46 (m, 2H), 3.18 (t, J=10.2 Hz, 1H), 3.02-2.87 (m, 2H), 2.86-2.75 (m, 2H), 2.74-2.65 (m, 1H), 2.59 (d, J=10.1 Hz, 2H), 2.47-2.41 (m, 1H), 2.27 (t, J=11.0 Hz, 1H), 2.19-2.08 (m, 1H), 1.96-1.86 (m, 1H), 1.73 (d, J=12.8 Hz, 1H), 1.57 (d, J=11.7 Hz, 6H), 0.95-0.85 (m, 6H). MS (ESI) m/z 675.5 [M+H]⁺.

Example 169: 4-(4-fluorobenzyl)-8,8-dimethyl-6-(2-((2R,5R)-5-methyl-2-(((R)-3-methylmorpholino)methyl)piperazin-1-yl)acetyl)-2-(pyrimidin-2-ylmethyl)-2,6,7,8-tetrahydro-1H-pyrrolo[2,3-e][1,2,4]triazolo[4,3-a]pyridin-1-one (compound 221)

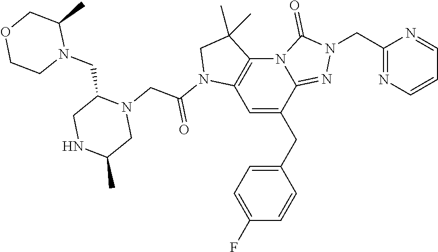 [SMILES: C[C@@H]1CN(CC(=O)N2CC(C)(C)c3c2cc(Cc2ccc(F)cc2)c2nn(Cc4ncccn4)c(=O)n32)[C@@H](CN2CCOC[C@H]2C)CN1]

Following the synthesis of compound 185, compound 221 was obtained as a white solid (37.5 mg). ¹H NMR (400 MHz, DMSO-d⁶) δ (ppm) 8.80 (d, J=4.9 Hz, 2H), 7.98 (s, 1H), 7.47 (t, J=4.9 Hz, 1H), 7.30 (dd, J=8.3, 5.7 Hz, 2H), 7.09 (t, J=8.8 Hz, 2H), 5.34 (s, 2H), 4.00 (d, J=10.6 Hz, 1H), 3.93-3.88 (m, 2H), 3.79 (d, J=10.7 Hz, 1H), 3.71 (d, J=16.7 Hz, 1H), 3.59 (d, J=16.7 Hz, 1H), 3.51-3.48 (m, 2H), 3.18 (t, J=9.9 Hz, 1H), 2.98-2.89 (m, 2H), 2.84-2.75 (m, 2H), 2.69 (d, J=9.7 Hz, 1H), 2.63-2.57 (m, 2H), 2.44 (t, J=10.9 Hz, 1H), 2.29-2.22 (m, 1H), 2.18-2.10 (m, 1H), 1.91 (t, J=9.6 Hz, 1H), 1.73 (d, J=12.6 Hz, 1H), 1.58 (d, J=11.9 Hz, 6H), 0.90-0.86 (m, 6H). MS (ESI) m/z 657.6 [M+H]⁺.

Example 170: 2-((2S,5R)-2-((3-oxa-8-azabicyclo[3.2.1]octan-8-yl)methyl)-5-methylpiperazin-1-yl)-1-(4-(4-fluorobenzyl)-8,8-dimethyl-7,8-dihydro-6H-pyrrolo[2,3-e][1,2,4]triazolo[1,5-a]pyridin-6-yl)ethan-1-one (compound 222)

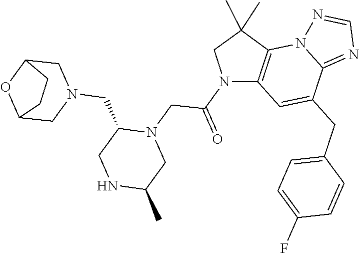 [SMILES: C[C@@H]1CN(CC(=O)N2CC(C)(C)c3c2cc(Cc2ccc(F)cc2)c2ncnn32)[C@@H](CN2CC3CCC(C2)O3)CN1]

Following the synthesis of compound 174, compound 222 was obtained as a white solid (55 mg). ¹H NMR (400 MHz, DMSO-d⁶) δ (ppm) 8.51 (s, 1H), 8.42 (s, 1H), 7.39 (dd, J=8.4, 5.7 Hz, 2H), 7.11 (t, J=8.9 Hz, 2H), 4.29 (s, 2H), 4.17 (d, J=10.6 Hz, 1H), 4.01 (d, J=10.6 Hz, 1H), 3.80 (d, J=16.5 Hz, 1H), 3.62 (d, J=16.5 Hz, 1H), 3.32-3.27 (m, 2H), 3.28-3.19 (m, 2H), 2.96 (dd, J=15.8, 4.5 Hz, 2H), 2.83-2.71 (m, 2H), 2.67-2.55 (m, 2H), 2.43-2.21 (m, 3H), 2.02 (dd, J=13.0, 3.0 Hz, 1H), 1.84-1.67 (m, 2H), 1.63-1.53 (m, 8H), 0.88 (d, J=6.1 Hz, 3H). MS (ESI) m/z 562.4 [M+H]⁺.

Example 171: methyl (R)-4-(((2S,5R)-1-(2-(4-(4-fluorobenzyl)-8,8-dimethyl-7,8-dihydro-6H-pyrrolo[2,3-e][1,2,4]triazolo[1,5-a]pyridin-6-yl)-2-oxoethyl)-5-methylpiperazin-2-yl)methyl)-3-methylpiperazine-1-carboxylate (compound 223)

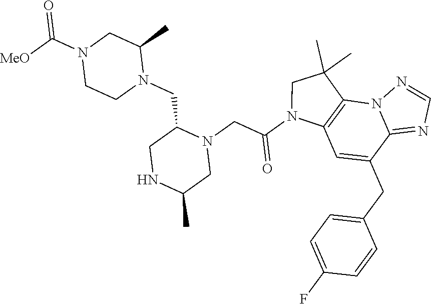 [SMILES: COC(=O)N1CCN(C[C@H]2CN[C@H](C)CN2CC(=O)N2CC(C)(C)c3c2cc(Cc2ccc(F)cc2)c2ncnn32)[C@H](C)C1]

Following the synthesis of compound 174, compound 223 was obtained as a white solid (29 mg). MS (ESI) m/z 606.7 [M+H]⁺.

Example 172: 1-(2-acetyl-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 224)

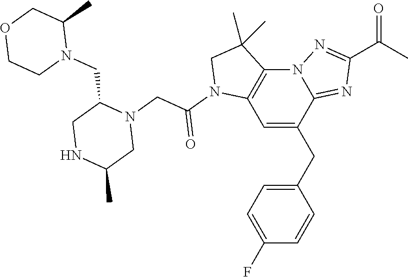 [SMILES: CC(=O)c1nc2c(Cc3ccc(F)cc3)cc3c(n2n1)C(C)(C)CN3C(=O)CN1C[C@@H](C)NC[C@@H]1CN1CCOC[C@H]1C]

MS (ESI) m/z 592.6 [M+H]⁺. ¹H NMR (400 MHz, DMSO-d⁶) δ (ppm) 8.46 (s, 1H), 7.47-7.39 (m, 2H), 7.17 (t, J=8.8 Hz, 2H), 4.38 (d, J=3.4 Hz, 2H), 4.20 (d, J=10.5 Hz, 1H), 4.00 (d, J=10.6 Hz, 1H), 3.87 (d, J=16.7 Hz, 1H), 3.69 (d, J=16.8 Hz, 1H), 3.53-3.38 (m, 2H), 3.17 (t, J=10.2 Hz, 1H), 3.06-2.98 (m, 1H), 2.97-2.77 (m, 3H), 2.76 (s, 3H), 2.67 (d, J=10.3 Hz, 2H), 2.56-2.54 (m, 3H), 2.32 (d, J=11.2 Hz, 1H), 2.18 (s, 1H), 1.96 (t, J=10.5 Hz, 1H), 1.80 (d, J=12.9 Hz, 1H), 1.66 (s, 3H), 1.62 (s, 3H), 0.96-0.90 (m, 6H).

Example 173: methyl (4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)(methyl)carbamate (compound 225)

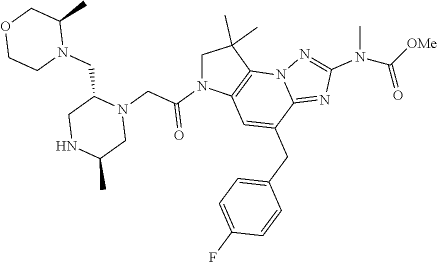 [SMILES: COC(=O)N(C)c1nc2c(Cc3ccc(F)cc3)cc3c(n2n1)C(C)(C)CN3C(=O)CN1C[C@@H](C)NC[C@@H]1CN1CCOC[C@H]1C]

Following the synthesis of compound 195, compound 225 was obtained as a white solid. MS (ESI) m/z 637.6 [M+H]⁺. ¹H NMR (400 MHz, DMSO-d⁶) δ (ppm) 8.35 (s, 1H), 7.43 (dd, J=8.5, 5.7 Hz, 2H), 7.16 (t, J=8.5 Hz, 2H), 4.26 (d, J=3.6 Hz, 2H), 4.14 (d, J=10.6 Hz, 1H), 3.94 (d, J=10.5 Hz, 1H), 3.80 (d, J=17.0 Hz, 1H), 3.75 (s, 3H), 3.55-3.44 (m, 3H), 3.41 (s, 3H), 3.14 (t, J=10.4 Hz, 2H), 2.95-2.79 (m, 4H), 2.76-2.61 (m, 2H), 2.57-2.53 (m, 2H), 2.36 (s, 1H), 2.18 (s, 1H), 1.94 (d, J=8.9 Hz, 1H), 1.81 (d, J=13.9 Hz, 1H), 1.62 (s, 3H), 1.58 (s, 3H), 1.02-0.88 (m, 6H).

Example 174: 1-(2-((3R,4R)-3,4-dihydroxypyrrolidin-1-yl)-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 226)

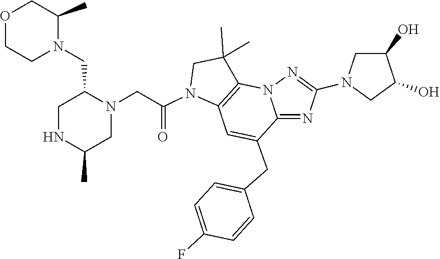 [SMILES: C[C@@H]1CN(CC(=O)N2CC(C)(C)c3c2cc(Cc2ccc(F)cc2)c2nc(N4C[C@@H](O)[C@H](O)C4)nn32)[C@@H](CN2CCOC[C@H]2C)CN1]

Following the synthesis of compound 195, compound 226 was obtained as a white solid. MS (ESI) m/z 651.6 [M+H]⁺. ¹H NMR (400 MHz, DMSO-d⁶) δ (ppm) 8.15 (s, 1H), 7.40 (dd, J=8.5, 5.7 Hz, 2H), 7.17-7.08 (m, 2H), 5.13 (d, J=3.2 Hz, 2H), 4.16 (s, 2H), 4.11 (d, J=10.6 Hz, 1H), 4.06 (s, 2H), 3.90 (d, J=10.6 Hz, 1H), 3.82 (d, J=16.5 Hz, 1H), 3.66 (d, J=3.9 Hz, 1H), 3.65-3.59 (m, 2H), 3.49 (s, 1H), 3.43 (d, J=10.8 Hz, 2H), 3.18 (t, J=10.4 Hz, 1H), 3.02-2.80 (m, 3H), 2.76 (s, 1H), 2.65 (d, J=10.7 Hz, 2H), 2.56-2.53 (m, 2H), 2.52-2.43 (m, 2H), 2.38-2.26 (m, 1H), 2.17 (s, 1H), 1.95 (t, J=9.6 Hz, 1H), 1.78 (d, J=12.9 Hz, 1H), 1.62 (s, 3H), 1.56 (s, 3H), 0.92 (d, J=6.2 Hz, 6H).

Example 175: 4-(4-fluorobenzyl)-2-(2-methoxyethyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 227)

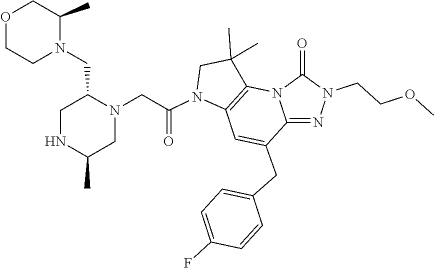 [SMILES: COCCn1nc2c(Cc3ccc(F)cc3)cc3c(n2c1=O)C(C)(C)CN3C(=O)CN1C[C@@H](C)NC[C@@H]1CN1CCOC[C@H]1C]

Following the synthesis of compound 185, compound 227 was obtained as a white solid (78.1 mg). MS (ESI) m/z 624.5[M+H]⁺. ¹H NMR (400 MHz, DMSO-d⁶) δ (ppm) 7.99 (s, 1H), 7.43-7.31 (m, 2H), 7.14 (t, J=8.8 Hz, 2H), 4.10-4.02 (m, 2H), 4.01-3.91 (m, 3H), 3.79 (d, J=10.4 Hz, 1H), 3.74-3.59 (m, 4H), 3.50 (d, J=10.2 Hz, 2H), 3.26-3.13 (m, 5H), 3.11-2.97 (m, 1H), 2.97-2.74 (m, 4H), 2.71-2.59 (m, 2H), 2.38-2.25 (m, 2H), 2.21-2.08 (m, 1H), 2.03-1.89 (m, 2H), 1.77 (d, J=12.8 Hz, 1H), 1.58 (d, J=12.3 Hz, 6H), 0.94-0.86 (m, 6H).

Example 176: 4-(4-fluorobenzyl)-8,8-dimethyl-6-(2-((2R,5R)-5-methyl-2-(((R)-3-methyl morpholino)methyl)piperazin-1-yl)acetyl)-2-((R)-tetrahydrofuran-3-yl)-2,6,7,8-tetrahydro-1H-pyrrolo[2,3-e][1,2,4]triazolo[4,3-a]pyridin-1-one (compound 228)

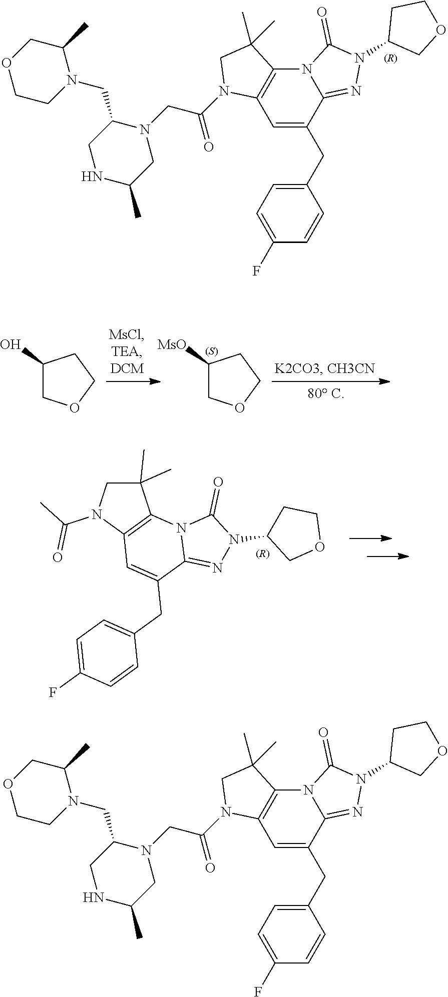

Step 1: (S)-tetrahydrofuran-3-yl methanesulfonate

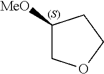

To a solution of (S)-tetrahydrofuran-3-ol (200 mg, 2.270 mmol, 1.0 eq) in CH₂Cl₂ (15 mL) were added triethylamine (689 mg, 6.81 mmol, 3.0 eq) and methanesulfonyl chloride (390 mg, 3.40 mmol, 1.5 eq) added at 0° C. Then the mixture was stirred at 25° C. for 5 h under N₂. To the reaction mixture were added water and DCM. The combined organic layer was washed with brine (100 mL), dried over Na₂SO₄, filtered and concentrated to give the title compound (377 mg, 100%) as a light oil.

Step 2: (R)-6-acetyl-4-(4-fluorobenzyl)-8,8-dimethyl-2-(tetrahydrofuran-3-yl)-2,6,7,8-tetrahydro-1H-pyrrolo[2,3-e][1,2,4]triazolo[4,3-a]pyridin-1-one

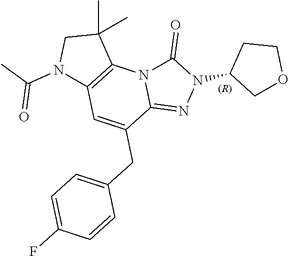

To a solution of 6-acetyl-4-(4-fluorobenzyl)-8,8-dimethyl-2,6,7,8-tetrahydro-1H-pyrrolo[2,3-e][1,2,4]triazolo[4,3-a]pyridin-1-one (200 mg, 0.564 mmol, 1.0 eq) in acetonitrile (15 mL) were added (S)-tetrahydrofuran-3-yl methanesulfonate (141 mg, 0.847 mmol, 1.5 eq) and K₂CO₃ (234 mg, 1.693 mmol, 3.0 eq) at 25° C. The mixture was stirred at 80° C. for 16 h under N₂. To the reaction mixture were added water and ethyl acetate. The combined organic layer was washed with brine (100 mL), dried over Na₂SO₄, filtered and concentrated. The residue was purified by silica gel flash column chromatography eluting with DCM/CH3OH=50/1 to give the title compound (200 mg, 83%) as a yellow solid. MS (ESI) m/z 425.4[M+H]+.

Following Steps: following the synthesis of compound 14, compound 228 was obtained as a white solid (43.6 mg). MS (ESI) m/z 636.6[M+H]⁺. ¹H NMR (400 MHz, DMSO-d⁶) δ (ppm) 8.01 (s, 1H), 7.43-7.32 (m, 2H), 7.21-7.04 (m, 2H), 5.08-4.94 (m, 1H), 4.06-3.93 (m, 5H), 3.91-3.82 (m, 2H), 3.78 (d, J=10.8 Hz, 1H), 3.74-3.58 (m, 2H), 3.50 (d, J=10.4 Hz, 2H), 3.21-3.11 (m, 1H), 3.09-2.97 (m, 1H), 2.95-2.71 (m, 4H), 2.69-2.58 (m, 2H), 2.56-2.53 (m, 1H), 2.37-2.21 (m, 3H), 2.21-2.09 (m, 1H), 1.99-1.87 (m, 1H), 1.75 (d, J=11.8 Hz, 1H), 1.57 (d, J=11.9 Hz, 6H), 0.94-0.87 (m, 6H).

Example 177: 1-(2-((3R,4S)-3,4-dihydroxypyrrolidin-1-yl)-4-(4-fluorobenzyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (compound 229)

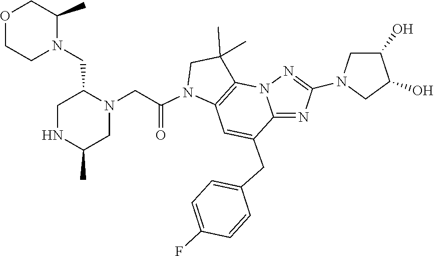 [SMILES: C[C@@H]1CN(CC(=O)N2CC(C)(C)c3c2cc(Cc2ccc(F)cc2)c2nc(N4C[C@@H](O)[C@@H](O)C4)nn32)[C@@H](CN2CCOC[C@H]2C)CN1]

Following the synthesis of compound 195, compound 229 was obtained as a white solid. MS (ESI) m/z 651.6 [M+H]⁺. ¹H NMR (400 MHz, DMSO-d⁶) δ (ppm) 8.15 (s, 1H), 7.44-7.36 (m, 2H), 7.18-7.09 (m, 2H), 4.94 (d, J=4.3 Hz, 2H), 4.17 (d, J=3.1 Hz, 4H), 4.10 (d, J=10.6 Hz, 1H), 3.91 (d, J=10.6 Hz, 1H), 3.81 (d, J=16.6 Hz, 1H), 3.69-3.59 (m, 3H), 3.49 (t, J=9.5 Hz, 2H), 3.39 (s, 1H), 3.17 (t, J=10.4 Hz, 1H), 3.05 (s, 1H), 2.97-2.78 (m, 4H), 2.74-2.64 (m, 2H), 2.60-2.54 (m, 3H), 2.45-2.28 (m, 1H), 2.19 (d, J=8.9 Hz, 1H), 2.01-1.91 (m, 1H), 1.80 (d, J=13.1 Hz, 1H), 1.61 (s, 3H), 1.57 (s, 3H), 0.94 (t, J=6.6 Hz, 6H).

Example 178: 4-(4-fluorobenzyl)-8,8-dimethyl-6-(2-((2R,5R)-5-methyl-2-(((R)-3-methylmorpholino)methyl)piperazin-1-yl)acetyl)-2-(pyrimidin-2-yl)-2,6,7,8-tetrahydro-1H-pyrrolo[2,3-e][1,2,4]triazolo[4,3-a]pyridin-1-one (compound 230)

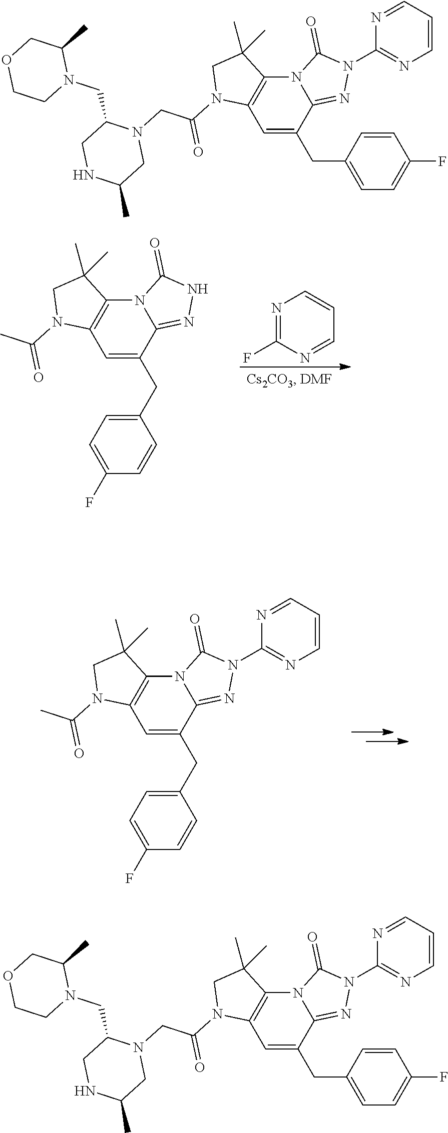

Step 1: 6-acetyl-4-(4-fluorobenzyl)-8,8-dimethyl-2-(pyrimidin-2-yl)-2,6,7,8-tetrahydro-1H-pyrrolo[2,3-e][1,2,4]triazolo[4,3-a]pyridin-1-one

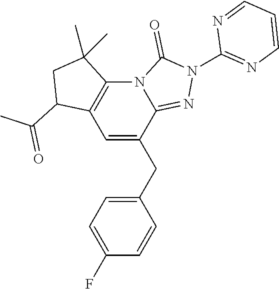 [SMILES: CC(=O)C1CC(C)(C)c2c1cc(Cc1ccc(F)cc1)c1nn(-c3ncccn3)c(=O)n21]

A mixture of 6-acetyl-4-(4-fluorobenzyl)-8,8-dimethyl-2,6,7,8-tetrahydro-1H-pyrrolo[2,3-e][1,2,4]triazolo[4,3-a]pyridin-1-one (200 mg, 0.564 mmol, 1.0 eq), 2-fluoropyrimidine (277 mg, 2.82 mmol, 5.0 eq) and Cs₂CO₃ (276 mg, 0.847 mmol, 1.5 eq) in DMF (5 mL) was stirred at 90° C. for 16 h. The mixture was diluted with water, extracted with ethyl acetate (twice). The organic layer was washed with brine, dried over Na₂SO₄ and concentrated to give the title compound (200 mg, 82%) as a yellow oil. MS (ESI) m/z 433.42 [M+H]⁺

Following steps: following the synthesis of compound 274, compound 230 was obtained as a white solid (4.5 mg). MS (ESI) m/z 644.63 [M+H]⁺

Example 179: 4-(4-fluorobenzyl)-2-(2-hydroxy-2-methylpropyl)-8,8-dim ethyl-6-(2-((2R,5R)-5-methyl-2-(((R)-3-methylmorpholino)methyl)piperazin-1-yl) acetyl)-2,6,7,8-tetrahydro-1H-pyrrolo[2,3-e][1,2,4]triazolo[4,3-a]pyridin-1-one (compound 231)

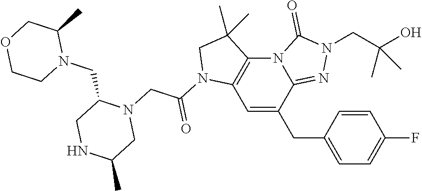 [SMILES: C[C@@H]1CN(CC(=O)N2CC(C)(C)c3c2cc(Cc2ccc(F)cc2)c2nn(CC(C)(C)O)c(=O)n32)[C@@H](CN2CCOC[C@H]2C)CN1]

Following the synthesis of compound 274, compound 231 was obtained as a white solid (32 mg). ¹H NMR (400 MHz, DMSO-d⁶) δ (ppm) 8.04 (s, 1H), 7.39 (dd, J=8.3, 5.8 Hz, 2H), 7.14 (t, J=8.8 Hz, 2H), 4.68 (s, 1H), 4.05-3.94 (m, 3H), 3.88-3.78 (m, 3H), 3.78-3.58 (m, 2H), 3.57-3.49 (m, 2H), 3.32-3.28 (m, 1H), 3.28-3.18 (m, 1H), 3.03-2.58 (m, 7H), 2.51-2.44 (m, 1H), 2.35-2.26 (m, 1H), 2.23-2.13 (m, 1H), 2.00-1.90 (m, 1H), 1.81-1.72 (m, 1H), 1.60 (d, J=12.7 Hz, 6H), 1.18 (s, 6H), 0.96-0.88 (m, 6H). MS (ESI) m/z 638.63 [M+H]⁺

Example 180: 4-(4-fluorobenzyl)-8,8-dimethyl-2-((1-methyl-1H-1,2,4-triazol-3-yl)methyl)-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 232)

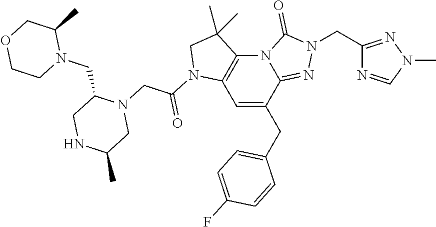 [SMILES: C[C@@H]1CN(CC(=O)N2CC(C)(C)c3c2cc(Cc2ccc(F)cc2)c2nn(Cc4ncn(C)n4)c(=O)n32)[C@@H](CN2CCOC[C@H]2C)CN1]

Following the synthesis of compound 185, compound 232 was obtained as a white solid (51.2 mg). ¹H NMR (400 MHz, DMSO-d⁶) δ (ppm) 8.42 (s, 1H), 7.96 (s, 1H), 7.32 (dd, J=8.5, 5.7 Hz, 2H), 7.10 (t, J=8.9 Hz, 2H), 5.12 (s, 2H), 3.99 (d, J=10.6 Hz, 1H), 3.96-3.86 (m, 2H), 3.84 (s, 3H), 3.79 (d, J=10.7 Hz, 1H), 3.72 (d, J=16.6 Hz, 1H), 3.57 (d, J=16.6 Hz, 1H), 3.49 (d, J=11.0 Hz, 2H), 3.18 (t, J=9.9 Hz, 1H), 2.92 (t, J=9.7 Hz, 2H), 2.87-2.76 (m, 2H), 2.68 (d, J=9.3 Hz, 1H), 2.57 (d, J=8.4 Hz, 2H), 2.41 (t, J=10.9 Hz, 1H), 2.25 (t, J=11.0 Hz, 1H), 2.13 (s, 1H), 1.90 (t, J=9.4 Hz, 1H), 1.72 (d, J=12.1 Hz, 1H), 1.58 (d, J=12.0 Hz, 6H), 0.87 (dd, J=10.9, 6.2 Hz, 6H). MS (ESI) m/z 661.4 [M+H]⁺.

Example 181: 2-(((R)-1,4-dioxan-2-yl)methyl)-4-(4-fluorobenzyl)-8,8-di methyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 233)

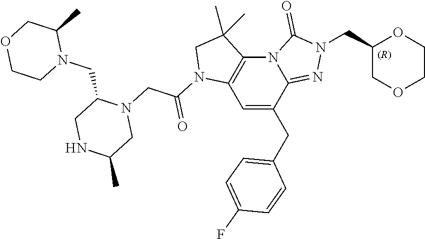

Following the synthesis of compound 185, compound 233 was obtained as a white solid (47.7 mg). MS (ESI) m/z 666.6[M+H]⁺. H NMR (400 MHz, DMSO-d⁶) δ (ppm) 8.00 (s, 1H), 7.43-7.31 (m, 2H), 7.19-7.06 (m, 2H), 4.05-3.90 (m, 5H), 3.88-3.59 (m, 7H), 3.55-3.45 (m, 4H), 3.42-3.36 (m, 1H), 3.22-3.11 (m, 1H), 3.06-2.96 (m, 1H), 2.96-2.88 (m, 1H), 2.88-2.70 (m, 3H), 2.70-2.55 (m, 2H), 2.49-2.45 (m, 1H), 2.37-2.22 (m, 1H), 2.18-2.07 (m, 1H), 1.99-1.86 (m, 1H), 1.75 (d, J=12.8 Hz, 1H), 1.57 (d, J=12.2 Hz, 6H), 0.93-0.86 (m, 6H).

Example 182: methyl (4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)carbamate (compound 234)

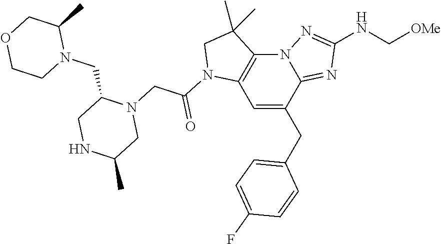 [SMILES: COCNc1nc2c(Cc3ccc(F)cc3)cc3c(n2n1)C(C)(C)CN3C(=O)CN1C[C@@H](C)NC[C@@H]1CN1CCOC[C@H]1C]

Following the synthesis of compound 195, compound 234 was obtained as a white solid. MS (ESI) m/z 623.6 [M+H]. ¹H NMR (400 MHz, DMSO-d⁶) δ (ppm) 10.57 (s, 1H), 8.31 (s, 1H), 7.40 (dd, J=8.4, 5.5 Hz, 2H), 7.15 (t, J=8.8 Hz, 2H), 4.24 (d, J=2.6 Hz, 2H), 4.15 (d, J=10.6 Hz, 1H), 3.95 (d, J=10.5 Hz, 1H), 3.84 (d, J=16.6 Hz, 1H), 3.71 (s, 3H), 3.66 (d, J=16.6 Hz, 1H), 2.56-2.45 (m, 3H), 3.18 (t, J=10.5 Hz, 1H), 3.01 (s, 1H), 2.97-2.72 (m, 4H), 2.70-2.61 (m, 2H), 2.47 (d, J=10.8 Hz, 1H), 2.33 (t, J=11.3 Hz, 1H), 2.18 (s, 1H), 2.06-1.90 (m, 1H), 1.79 (d, J=12.8 Hz, 1H), 1.63 (s, 3H), 1.58 (s, 3H), 0.92 (t, J=5.4 Hz, 6H).

Example 183: 1-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)-3-methylimidazolidin-2-one (compound 235)

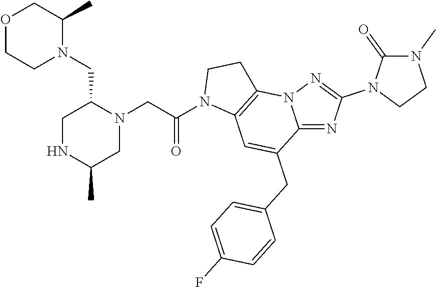 [SMILES: C[C@@H]1CN(CC(=O)N2CCc3c2cc(Cc2ccc(F)cc2)c2nc(N4CCN(C)C4=O)nn32)[C@@H](CN2CCOC[C@H]2C)CN1]

Following the synthesis of compound 195, compound 235 was obtained as a white solid. MS (ESI) m/z 648.6 [M+H]⁺. ¹H NMR (400 MHz, DMSO-d⁶) δ (ppm) 8.30 (s, 1H), 7.47-7.38 (m, 2H), 7.19-7.09 (m, 2H), 4.24 (d, J=2.8 Hz, 2H), 4.16 (d, J=10.6 Hz, 1H), 4.00 (dd, J=9.1, 6.8 Hz, 2H), 3.95 (d, J=10.6 Hz, 1H), 3.85 (d, J=16.6 Hz, 1H), 3.64 (d, J=16.5 Hz, 1H), 3.58-3.46 (m, 5H), 3.18 (t, J=10.1 Hz, 1H), 3.00-2.84 (m, 2H), 2.83 (s, 3H), 2.75 (dd, J=11.8, 2.7 Hz, 1H), 2.68-2.60 (m, 2H), 2.58-2.53 (m, 2H), 2.46 (t, J=11.0 Hz, 1H), 2.32 (t, J=11.0 Hz, 1H), 2.18 (s, 1H), 2.00-1.90 (m, 1H), 1.78 (d, J=12.7 Hz, 1H), 1.63 (s, 3H), 1.58 (s, 3H), 0.92 (t, J=6.7 Hz, 6H).

Example 184: 4-(4-fluorobenzyl)-8,8-dimethyl-6-(2-((2R,5R)-5-methyl-2-(((R)-3-methylmorpholino)methyl)piperazin-1-yl)acetyl)-2-(pyrimidin-5-ylmethyl)-2,6,7,8-tetrahydro-1H-pyrrolo[2,3-e][1,2,4]triazolo[4,3-a]pyridin-1-one (compo und 236)

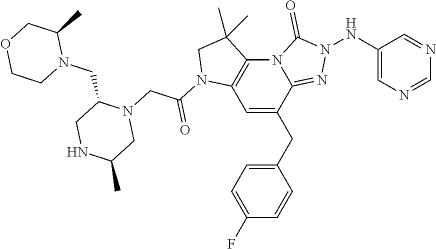 [SMILES: C[C@@H]1CN(CC(=O)N2CC(C)(C)c3c2cc(Cc2ccc(F)cc2)c2nn(Nc4cncnc4)c(=O)n32)[C@@H](CN2CCOC[C@H]2C)CN1]

Following the synthesis of compound 185, compound 236 was obtained as a white solid (52.8 mg). ¹H NMR (400 MHz, DMSO-d⁶) δ (ppm) 9.16 (s, 1H), 8.83 (s, 2H), 8.00 (s, 1H), 7.31 (dd, J=8.4, 5.7 Hz, 2H), 7.09 (t, J=8.8 Hz, 2H), 5.22 (s, 2H), 3.98 (d, J=10.5 Hz, 1H), 3.96-3.89 (m, 2H), 3.78 (d, J=10.6 Hz, 1H), 3.72 (d, J=16.5 Hz, 1H), 3.59 (d, J=16.7 Hz, 1H), 3.49 (d, J=11.1 Hz, 2H), 3.18 (t, J=10.1 Hz, 1H), 3.01-2.87 (m, 2H), 2.87-2.75 (m, 2H), 2.70 (t, J=10.3 Hz, 1H), 2.59 (d, J=10.8 Hz, 2H), 2.48-2.42 (m, 1H), 2.37-2.23 (m, 1H), 2.18-2.08 (m, 1H), 1.91 (t, J=9.5 Hz, 1H), 1.73 (d, J=12.3 Hz, 1H), 1.57 (d, J=12.9 Hz, 6H), 0.90-0.85 (m, 6H). MS (ESI) m/z 657.5 [M+H]⁺.

Example 185: 2-(((S)-1,4-dioxan-2-yl)methyl)-4-(4-fluorobenzyl)-8,8-dim ethyl-6-(2-((2R,5R)-5-methyl-2-(((R)-3-methylmorpholino)methyl)piperazin-1-yl) acetyl)-2,6,7,8-tetrahydro-1H-pyrrolo[2,3-e][1,2,4]triazolo[4,3-a]pyridin-1-one (compound 237)

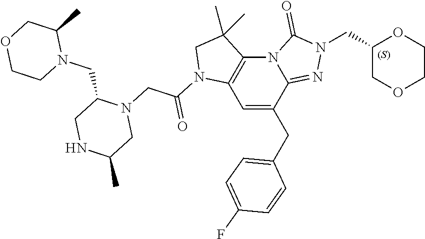

Following the synthesis of compound 185, compound 237 was obtained as a white solid (55.2 mg). MS (ESI) m/z 666.6[M+H]⁺. ¹H NMR (400 MHz, DMSO-d⁶) δ (ppm) 7.99 (s, 1H), 7.41-7.28 (m, 2H), 7.16-7.04 (m, 2H), 4.06-3.89 (m, 5H), 3.85-3.79 (m, 1H), 3.78-3.70 (m, 3H), 3.68-3.56 (m, 3H), 3.52-3.43 (m, 4H), 3.40-3.37 (m, 1H), 3.20-3.09 (m, 1H), 3.02-2.94 (m, 1H), 2.92-2.86 (m, 1H), 2.84-2.74 (m, 2H), 2.73-2.67 (m, 1H), 2.63-2.53 (m, 2H), 2.47-2.42 (m, 1H), 2.34-2.20 (m, 1H), 2.18-2.07 (m, 1H), 1.97-1.83 (m, 1H), 1.72 (d, J=12.7 Hz, 1H), 1.55 (d, J=13.1 Hz, 6H), 0.87 (d, J=5.7 Hz, 6H).

Example 186: N-(4-(4-fluorobenzyl)-8,8-dimethyl-6-(2-((2R,5R)-5-methy 1-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)-N-methylacetamide (compound 238)

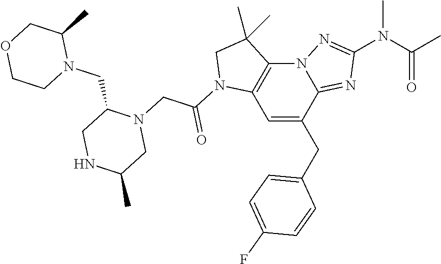 [SMILES: CC(=O)N(C)c1nc2c(Cc3ccc(F)cc3)cc3c(n2n1)C(C)(C)CN3C(=O)CN1C[C@@H](C)NC[C@@H]1CN1CCOC[C@H]1C]

Following the synthesis of compound 195, compound 238 was obtained as a white solid. MS (ESI) m/z 621.7 [M+H]⁺.

Example 187: N-(4-(4-fluorobenzyl)-8,8-dimethyl-6-(2-((2R,5R)-5-meth yl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)formamide (compound 239)

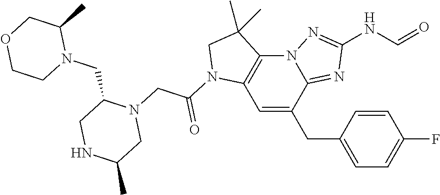 [SMILES: C[C@@H]1CN(CC(=O)N2CC(C)(C)c3c2cc(Cc2ccc(F)cc2)c2nc(NC=O)nn32)[C@@H](CN2CCOC[C@H]2C)CN1]

Following the synthesis of compound 195, compound 239 was obtained as a white solid (20.5 mg). ¹H NMR (400 MHz, DMSO-d⁶) δ (ppm) 9.21 (s, 1H), 8.33 (s, 1H), 7.40 (dd, J=8.3, 5.7 Hz, 2H), 7.15 (t, J=8.8 Hz, 2H), 4.30-4.19 (m, 2H), 4.15 (d, J=10.6 Hz, 1H), 3.95 (d, J=10.6 Hz, 1H), 3.83 (d, J=16.7 Hz, 1H), 3.70 (d, J=16.7 Hz, 1H), 3.54-3.43 (m, 3H), 3.22-3.02 (m, 2H), 2.95-2.77 (m, 4H), 2.74-2.64 (m, 2H), 2.60-2.54 (m, 2H), 2.36 (t, J=11.2 Hz, 1H), 2.25-2.12 (m, 1H), 2.01-1.89 (m, 1H), 1.86-1.74 (m, 1H), 1.60 (d, J=16.7 Hz, 6H), 1.01-0.82 (m, 6H). MS (ESI) m/z 607.62 [M+H]⁺

Example 188: 4-(4-fluorobenzyl)-2-((1-hydroxycyclopropyl)methyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 240)

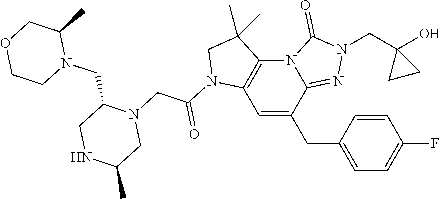 [SMILES: C[C@@H]1CN(CC(=O)N2CC(C)(C)c3c2cc(Cc2ccc(F)cc2)c2nn(CC4(O)CC4)c(=O)n32)[C@@H](CN2CCOC[C@H]2C)CN1]

Following the synthesis of compound 262, compound 240 was obtained as a yellow solid (17.5 mg). ¹H NMR (400 MHz, DMSO-d⁶) δ (ppm) 8.07 (s, 1H), 7.49-7.37 (m, 2H), 7.20-7.11 (m, 2H), 5.55 (s, 1H), 4.09-3.93 (m, 5H), 3.86-3.58 (m, 3H), 3.57-3.48 (m, 2H), 3.26-3.15 (m, 1H), 3.05-2.79 (m, 4H), 2.75-2.57 (m, 3H), 2.52-2.42 (m, 2H), 2.33-2.23 (m, 1H), 2.22-2.12 (m, 1H), 1.99-1.89 (m, 1H), 1.80-1.72 (m, 1H), 1.61 (d, J=13.5 Hz, 6H), 0.95-0.86 (m, 6H), 0.81-0.72 (m, 2H), 0.68-0.60 (m, 2H). MS (ESI) m/z 636.64 [M+H]⁺

Example 189: N-(4-(4-fluorobenzyl)-8,8-dimethyl-6-(2-((2R,5R)-5-methy 1-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)-N-methylformamide (compound 241)

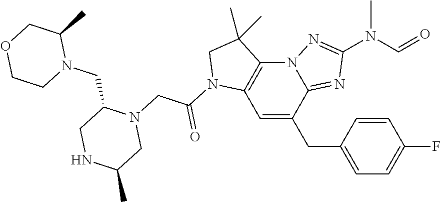 [SMILES: C[C@@H]1CN(CC(=O)N2CC(C)(C)c3c2cc(Cc2ccc(F)cc2)c2nc(N(C)C=O)nn32)[C@@H](CN2CCOC[C@H]2C)CN1]

Following the synthesis of compound 204, compound 241 was obtained as a yellow solid (55 mg).

¹H NMR (400 MHz, DMSO-d⁶) δ (ppm) 9.45 (s, 1H), 8.39 (s, 1H), 7.48-7.38 (m, 2H), 7.21-7.09 (m, 2H), 4.34-4.09 (m, 3H), 4.03-3.78 (m, 2H), 3.73-3.62 (m, 1H), 3.57-3.42 (m, 4H), 3.22-3.12 (m, 1H), 3.08-2.56 (m, 10H), 2.39-2.29 (m, 1H), 2.24-2.11 (m, 1H), 2.04-1.88 (m, 1H), 1.85-1.73 (m, 1H), 1.62 (d, J=17.0 Hz, 6H), 1.03-0.83 (m, 6H). MS (ESI) m/z 593.58 [M+H]⁺

Example 190: 2-(2,2-difluoro-3-hydroxypropyl)-4-(4-fluorobenzyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 242)

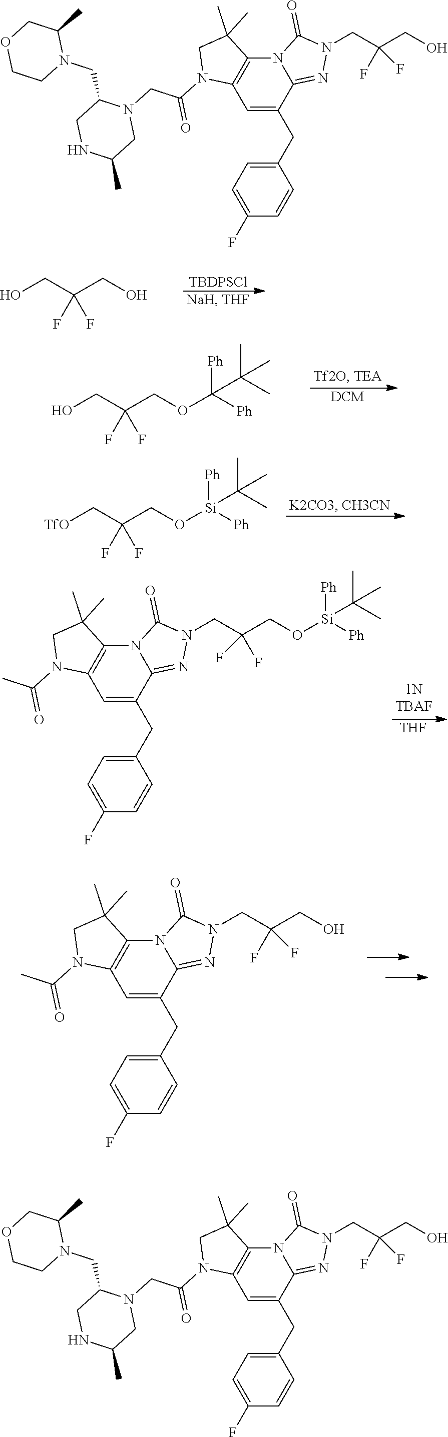

Step 1: 3-((tert-butyldiphenylsilyl)oxy)-2,2-difluoropropan-1-ol

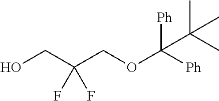 [SMILES: CC(C)(C)C(OCC(F)(F)CO)(c1ccccc1)c1ccccc1]

To a solution of 2,2-difluoropropane-1,3-diol (500 mg, 4.46 mmol, 1.0 eq) in THF (15 mL) was added sodium hydride (178 mg, 4.46 mmol, 1.0 eq) at 0° C. Then the mixture was stirred at 0° C. for 1 h. Tert-butylchlorodiphenylsilane (1226 mg, 4.46 mmol, 1.0 eq) was added at 0° C. and stirred at 25° C. for 3 h under N₂. Water and ethyl acetate were added. The combined organic layer was washed with brine (100 mL), dried over Na₂SO₄, filtered and concentrated. The residue was purified by silica gel flash column chromatography eluting with PE/EA=5/1 to give the title compound (887 mg, 56.7%) as a light oil.

Step 2: 3-((tert-butyldiphenylsilyl)oxy)-2,2-difluoropropyl trifluoromethanesulfonate

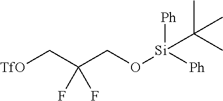 [SMILES: CC(C)(C)[Si](OCC(F)(F)COS(=O)(=O)C(F)(F)F)(c1ccccc1)c1ccccc1]

To a solution of 3-((tert-butyldiphenylsilyl)oxy)-2,2-difluoropropan-1-ol (350 mg, 0.999 mmol, 1.0 eq) in CH₂Cl₂ (15 mL) were added 2,6-dimethylpyridine (321 mg, 3.00 mmol, 3.0 eq) and trifluoromethanesulfonic anhydride (563 mg, 1.997 mmol, 2.0 eq) at 0° C. The mixture was stirred at 25° C. for 3 h under N₂. To the reaction mixture were added water and DCM. The combined organic layer was washed with brine (100 mL), dried over Na₂SO₄, filtered and concentrated to give the title compound (620 mg, 129%) as a light oil.

Step 3: 6-acetyl-2-(3-((tert-butyldiphenylsilyl)oxy)-2,2-difluoropropyl)-4-(4-fluorobenzyl)-8,8-dimethyl-2,6,7,8-tetrahydro-1H-pyrrolo[2,3-e][1,2,4]triazolo[4,3-a]pyridin-1-one

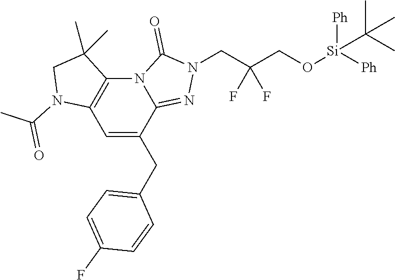 [SMILES: CC(=O)N1CC(C)(C)c2c1cc(Cc1ccc(F)cc1)c1nn(CC(F)(F)CO[Si](c3ccccc3)(c3ccccc3)C(C)(C)C)c(=O)n21]

To a solution of 6-acetyl-4-(4-fluorobenzyl)-8,8-dimethyl-2,6,7,8-tetrahydro-1H-pyrrolo[2,3-e][1,2,4]triazolo[4,3-a]pyridin-1-one (300 mg, 0.847 mmol, 1.0 eq) in acetonitrile (15 mL) were added 3-((tert-butyldiphenylsilyl)oxy)-2,2-difluoropropyl trifluoromethanesulfonate (613 mg, 1.270 mmol, 1.5 eq), and K₂CO₃ (351 mg, 2.54 mmol, 3.0 eq) at 25° C. The mixture was stirred at 80° C. for 16 h under N₂. Water and ethyl acetate were added. The combined organic layer was washed with brine (100 mL), dried over Na₂SO₄, filtered and concentrated. The residue was purified by silica gel flash column chromatography eluting with DCM/CH₃OH=50/1 to give the title compound (510 mg, 88%) as a yellow solid. MS (ESI) m/z 687.5[M+H]⁺.

Step 4: 6-acetyl-2-(2,2-difluoro-3-hydroxypropyl)-4-(4-fluorobenzyl)-8,8-dimethyl-2,6,7,8-tetrahydro-1H-pyrrolo[2,3-e][1,2,4]triazolo[4,3-a]pyridin-1-one

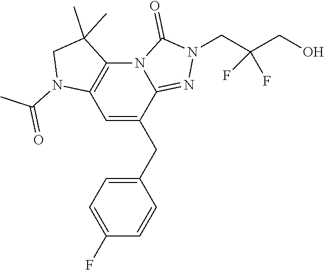 [SMILES: CC(=O)N1CC(C)(C)c2c1cc(Cc1ccc(F)cc1)c1nn(CC(F)(F)CO)c(=O)n21]

To a solution of 6-acetyl-2-(3-((tert-butyldiphenylsilyl)oxy)-2,2-difluoropropyl)-4-(4-fluoro benzyl)-8,8-dimethyl-2,6,7,8-tetrahydro-1H-pyrrolo[2,3-e][1,2,4]triazolo[4,3-a]pyridin-1-one (310 mg, 0.451 mmol, 1.0 eq) in THF (15 mL) was added 1N TBAF (0.68 mL, 0.677 mmol, 1.5 eq) at 25° C. The mixture was stirred at 25° C. for 16 h under N₂. To the reaction mixture were added water and ethyl acetate. The combined organic layer was washed with brine (100 mL), dried over Na₂SO₄, filtered and concentrated. The residue was purified by silica gel flash column chromatography eluting with DCM/CH3OH=50/1 to give the title compound (160 mg, 79%) as a yellow solid.

Following steps: following the synthesis of compound 14, compound 242 was obtained as a white solid (102 mg). MS (ESI) m/z 660.7[M+H]⁺. ¹H NMR (400 MHz, DMSO-d⁶) δ (ppm) 8.03 (s, 1H), 7.43-7.30 (m, 2H), 7.19-7.06 (m, 2H), 5.72-5.60 (m, 1H), 4.48-4.36 (m, 2H), 4.02-3.91 (m, 3H), 3.83-3.66 (m, 4H), 3.58 (d, J=16.7 Hz, 1H), 3.49 (d, J=11.0 Hz, 2H), 3.22-3.09 (m, 1H), 2.99-2.88 (m, 2H), 2.87-2.74 (m, 2H), 2.68 (d, J=11.6 Hz, 1H), 2.57 (d, J=8.7 Hz, 2H), 2.46-2.37 (m, 1H), 2.35-2.20 (m, 1H), 2.18-2.07 (m, 1H), 2.01-1.86 (m, 1H), 1.73 (d, J=12.9 Hz, 1H), 1.56 (d, J=12.2 Hz, 6H), 0.92-0.84 (m, 6H).

Example 191: 2-(2-(dimethylamino)ethyl)-4-(4-fluorobenzyl)-8,8-dimeth yl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 243)

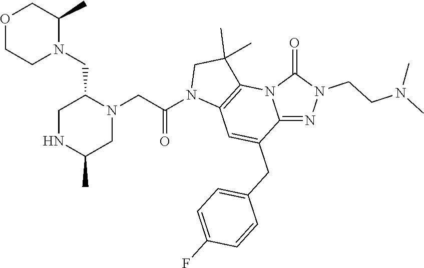 [SMILES: C[C@@H]1CN(CC(=O)N2CC(C)(C)c3c2cc(Cc2ccc(F)cc2)c2nn(CCN(C)C)c(=O)n32)[C@@H](CN2CCOC[C@H]2C)CN1]

Following the synthesis of compound 185, compound 243 was obtained as a white solid (6.5 mg). MS (ESI) m/z 637.6 [M+H]⁺.

Example 192: 1-(4-(4-fluorobenzyl)-2-(1-hydroxycyclopropyl)-8,8-dimethyl-7,8-dihydro-6H-pyrrolo[2,3-e][1,2,4]triazolo[1,5-a]pyridin-6-yl)-2-((2R,5R)-5-methyl-2-(((R)-3-methylmorpholino)methyl)piperazin-1-yl)ethan-1-one (Compound 244)

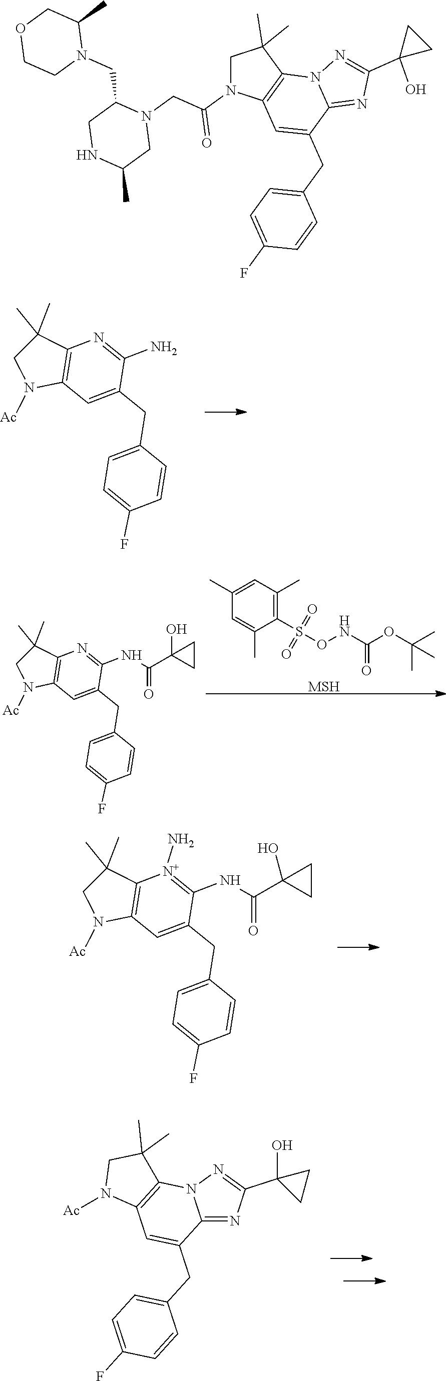

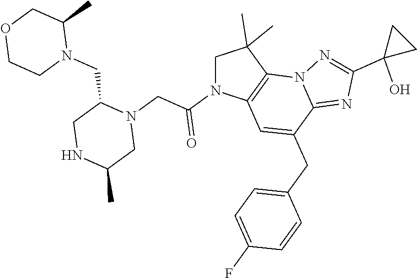 [SMILES: C[C@@H]1CN(CC(=O)N2CC(C)(C)c3c2cc(Cc2ccc(F)cc2)c2nc(C4(O)CC4)nn32)[C@@H](CN2CCOC[C@H]2C)CN1]

Step 1: N-(1-acetyl-6-(4-fluorobenzyl)-3,3-dimethyl-2,3-dihydro-1H-pyrrolo[3,2-b]pyridin-5-yl)-1-hydroxycyclopropane-1-carboxamide

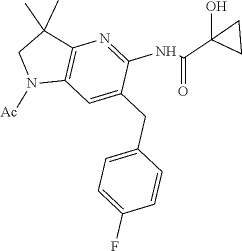 [SMILES: CC(=O)N1CC(C)(C)c2nc(NC(=O)C3(O)CC3)c(Cc3ccc(F)cc3)cc21]

To solution of 1-hydroxycyclopropane-1-carboxylic acid (204.2 mg, 2.0 mmol, 2.0 eq.) in 10.0 mL of THF was added DMF (0.2 mL) and SOCl₂ (416.5 mg, 3.5 mmol, 3.5 eq.) dropwise. The mixture was stirred at room temperature for 3.0 h. 1-(5-amino-6-(4-fluorobenzyl)-3,3-dimethyl-2,3-dihydro-1H-pyrrolo[3,2-b]pyridin-1-yl)ethan-1-one (313.38 mg, 1.0 eq., 1.0 mmol) was added and stirred for 16.0 h at room temperature. The solution was concentrated and the residue was used directly in next step.

Steps 2-3: 1-(4-(4-fluorobenzyl)-2-(1-hydroxycyclopropyl)-8,8-dimethyl-7,8-dihydro-6H-pyrrolo[2,3-e][1,2,4]triazolo[1,5-a]pyridin-6-yl)ethan-1-one

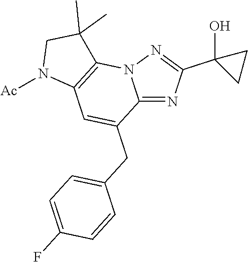 [SMILES: CC(=O)N1CC(C)(C)c2c1cc(Cc1ccc(F)cc1)c1nc(C3(O)CC3)nn21]

A solution of the tert-butyl N-[(2,4,6-trimethylbenzenesulfonyl)oxy]carbamate (6.30 g, 20.0 mmol, 20.0 eq.) in 15.0 mL of DCM was cooled drown to −30° C. TFA (10.0 mL) was added by dropwise stirred at −30° C.˜−15° C. for 2.0 h. The solution was then diluted in ice-water (300.0 mL) the white solid was separated by filtration and the cake was washed by ice-water twice. The cake was dissolved in 15.0 mL of DCM, dried by MgSO₄. After filtration, tert-butyl ((mesitylsulfonyl)oxy)carbamate (400 mg, 1.0 mmol, 1.0 eq.) was added and the solution was stirred at 0° C. for 3.5 hr. After concentration, DMAP (134.39 mg, 1.1 eq., 1.1 mmol), DIPEA (64.62 mg, 0.5 eq., 0.5 mmol), benzenesulfonamide (BSA, 628.76 mg, 4.0 eq., 4.0 mmol), and 30.0 mL MeCN were added into the mixture and the solution was further heated to reflux and stirred for 16.0 hr. The reaction was detected by LC-MS and purified by pre-HPLC to offer target compound. LC-MS: 395.27 [M+H]⁺. ¹H NMR (400 MHz, Chloroform-d) δ (ppm) 8.72 (s, 1H), 7.32-7.23 (m, 2H), 7.00 (t, J=8.5 Hz, 2H), 4.39 (s, 2H), 4.06 (s, 2H), 2.28 (s, 3H), 1.65 (s, 6H), 1.64-1.59 (m, 2H), 1.49 (d, J=3.0 Hz, 2H).

Following steps: following the synthesis of compound 14, compound 244 was synthesized as a white solid (5.5 mg). MS (ESI) m/z 606.5 [M+H]⁺.

Example 193: 4-(4-fluorobenzyl)-8,8-dimethyl-6-(2-((2R,5R)-5-methyl-2-(((R)-3-methylmorpholino)methyl)piperazin-1-yl)acetyl)-2-(pyridin-3-ylmethyl)-2,6,7,8-tetrahydro-1H-pyrrolo[2,3-e][1,2,4]triazolo[4,3-a]pyridin-1-one (compound 245)

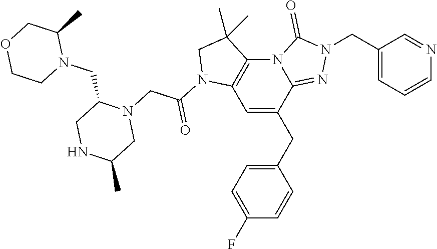 [SMILES: C[C@@H]1CN(CC(=O)N2CC(C)(C)c3c2cc(Cc2ccc(F)cc2)c2nn(Cc4cccnc4)c(=O)n32)[C@@H](CN2CCOC[C@H]2C)CN1]

Following the synthesis of compound 185, compound 245 was obtained as a white solid (12 mg). MS (ESI) m/z 657.5 [M+H]⁺.

Example 194: 4-(4-fluorobenzyl)-8,8-dimethyl-6-(2-((2R,5R)-5-methyl-2-(((R)-3-methylmorpholino)methyl)piperazin-1-yl)acetyl)-2-(pyrimidin-4-ylmethyl)-2,6,7,8-tetrahydro-1H-pyrrolo[2,3-e][1,2,4]triazolo[4,3-a]pyridin-1-one (compound 246)

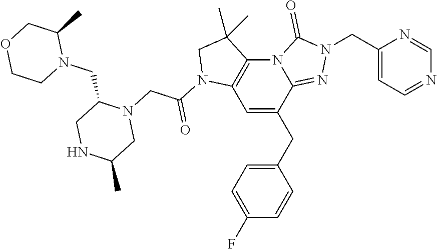 [SMILES: C[C@@H]1CN(CC(=O)N2CC(C)(C)c3c2cc(Cc2ccc(F)cc2)c2nn(Cc4ccncn4)c(=O)n32)[C@@H](CN2CCOC[C@H]2C)CN1]

Following the synthesis of compound 185, compound 246 was obtained as a white solid (56 mg). ¹H NMR (400 MHz, DMSO-d⁶) δ (ppm) 9.15 (d, J=1.1 Hz, 1H), 8.79 (d, J=5.2 Hz, 1H), 8.01 (s, 1H), 7.44 (d, J=5.1 Hz, 1H), 7.33-7.28 (m, 2H), 7.10 (t, J=8.8 Hz, 2H), 5.27 (s, 2H), 4.01 (d, J=10.6 Hz, 1H), 3.93 (s, 2H), 3.80 (d, J=10.7 Hz, 1H), 3.73 (d, J=16.6 Hz, 1H), 3.59 (d, J=16.6 Hz, 1H), 3.50 (d, J=10.6 Hz, 2H), 3.19 (t, J=9.9 Hz, 1H), 2.98-2.89 (m, 2H), 2.86-2.78 (m, 2H), 2.70 (d, J=9.6 Hz, 1H), 2.59 (t, J=8.4 Hz, 2H), 2.47-2.40 (m, 1H), 2.26 (t, J=11.0 Hz, 1H), 2.20-2.10 (m, 1H), 1.97-1.86 (m, 1H), 1.74 (d, J=12.8 Hz, 1H), 1.58 (d, J=12.4 Hz, 6H), 0.91-0.86 (m, 6H). MS (ESI) m/z 657.5 [M+H]⁺.

Example 195: 1-(4-(4-fluorobenzyl)-8,8-dimethyl-2-(1H-pyrazol-5-yl)-7,8-dihydro-6H-pyrrolo[2,3-e][1,2,4]triazolo[1,5-a]pyridin-6-yl)-2-((2R,5R)-5-methy 1-2-(((R)-3-methylmorpholino)methyl)piperazin-1-yl)ethan-1-one (compound 247)

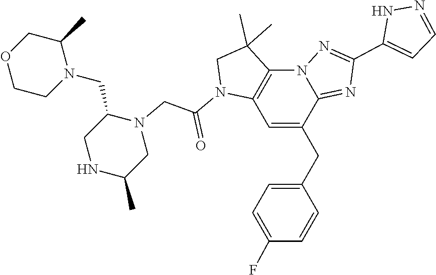 [SMILES: C[C@@H]1CN(CC(=O)N2CC(C)(C)c3c2cc(Cc2ccc(F)cc2)c2nc(-c4ccn[nH]4)nn32)[C@@H](CN2CCOC[C@H]2C)CN1]

Following the synthesis of compound 184, compound 247 was obtained as a white solid MS (ESI) m/z 616.6 [M+H]⁺. ¹H NMR (400 MHz, DMSO-d⁶) δ (ppm) 13.25 (s, 1H), 8.36 (s, 1H), 7.86 (s, 1H), 7.46 (dd, J=8.4, 5.6 Hz, 2H), 7.20-7.11 (m, 2H), 6.90 (d, J=2.2 Hz, 1H), 4.42-4.29 (m, 2H), 4.20 (d, J=10.6 Hz, 1H), 3.98 (d, J=10.5 Hz, 1H), 3.87 (d, J=16.6 Hz, 1H), 3.65 (d, J=16.5 Hz, 1H), 3.50 (d, J=11.2 Hz, 2H), 3.19 (t, J=10.3 Hz, 1H), 3.00-2.81 (m, 3H), 2.74 (d, J=11.3 Hz, 1H), 2.64 (d, J=9.3 Hz, 2H), 2.45 (t, J=11.0 Hz, 1H), 2.31 (t, J=11.0 Hz, 1H), 2.18 (s, 1H), 1.95 (t, J=10.6 Hz, 1H), 1.78 (d, J=13.0 Hz, 1H), 1.69 (s, 2H), 1.64 (s, 3H), 0.92 (dd, J=12.5, 6.0 Hz, 6H).

Example 196: 1-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)imidazolidin-2-one (compound 248)

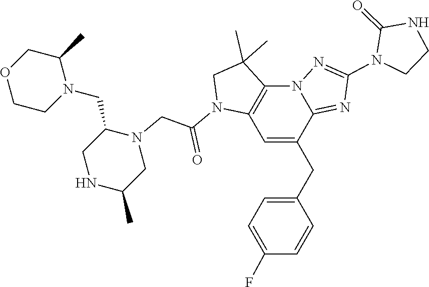 [SMILES: C[C@@H]1CN(CC(=O)N2CC(C)(C)c3c2cc(Cc2ccc(F)cc2)c2nc(N4CCNC4=O)nn32)[C@@H](CN2CCOC[C@H]2C)CN1]

Following the synthesis of compound 195, compound 248 was obtained as a white solid. MS (ESI) m/z 634.6 [M+H]⁺. ¹H NMR (400 MHz, DMSO-d⁶) δ (ppm) 8.29 (s, 1H), 7.43 (dd, J=8.4, 5.6 Hz, 2H), 7.20 (s, 1H), 7.14 (t, J=8.8 Hz, 2H), 4.25 (d, J=3.0 Hz, 2H), 4.23-4.12 (m, 1H), 4.06 (dd, J=9.0, 6.8 Hz, 2H), 3.95 (d, J=10.5 Hz, 1H), 3.85 (d, J=16.6 Hz, 1H), 3.64 (d, J=16.5 Hz, 1H), 3.49 (t, J=8.1 Hz, 4H), 3.17 (t, J=10.3 Hz, 1H), 3.02-2.79 (m, 4H), 2.74 (dd, J=11.9, 2.8 Hz, 1H), 2.68-2.58 (m, 2H), 2.55-2.53 (m, 2H), 2.46 (t, J=11.0 Hz, 1H), 2.31 (t, J=11.0 Hz, 1H), 2.17 (s, 1H), 2.00-1.90 (m, 1H), 1.78 (d, J=12.7 Hz, 1H), 1.63 (s, 3H), 1.59 (s, 3H), 0.92 (dd, J=8.2, 6.1 Hz, 6H).

Example 197: 2-(azetidin-3-yl)-4-(4-fluorobenzyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 249)

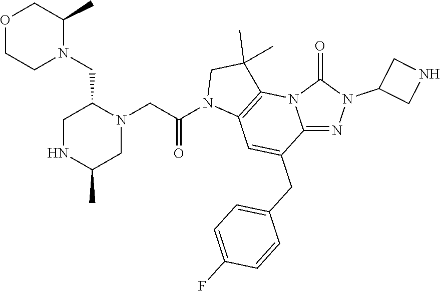 [SMILES: C[C@@H]1CN(CC(=O)N2CC(C)(C)c3c2cc(Cc2ccc(F)cc2)c2nn(C4CNC4)c(=O)n32)[C@@H](CN2CCOC[C@H]2C)CN1]

Following the synthesis of compound 135, compound 249 was obtained as a white solid (25.2 mg). MS (ESI) m/z 621.4 [M+H]⁺.

Example 198: 1-(4-(4-fluorobenzyl)-2-(methoxymethyl)-8,8-dimethyl-7,8-dihydro-6H-pyrrolo[2,3-e][1,2,4]triazolo[1,5-a]pyridin-6-yl)-2-((2R,5R)-5-methy 1-2-(((R)-3-methylmorpholino)methyl)piperazin-1-yl)ethan-1-one (compound 250)

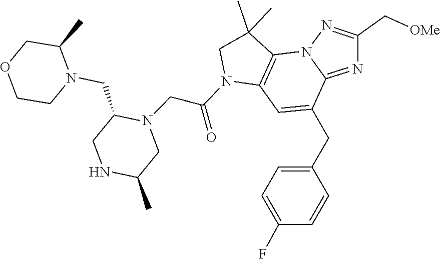 [SMILES: COCc1nc2c(Cc3ccc(F)cc3)cc3c(n2n1)C(C)(C)CN3C(=O)CN1C[C@@H](C)NC[C@@H]1CN1CCOC[C@H]1C]

Following the synthesis of compound 141, compound 250 was obtained as a white solid after lyophilization. MS (ESI) m/z 594.6 [M+H]⁺. ¹H NMR (400 MHz, DMSO-d⁶) δ (ppm) 8.37 (s, 1H), 7.41 (dd, J=8.4, 5.6 Hz, 2H), 7.15 (dd, J=9.9, 7.7 Hz, 2H), 4.66 (s, 2H), 4.31 (d, J=3.7 Hz, 2H), 4.17 (d, J=10.6 Hz, 1H), 3.96 (d, J=10.5 Hz, 1H), 3.84 (d, J=16.6 Hz, 1H), 3.67 (d, J=16.6 Hz, 1H), 3.49 (d, J=12.2 Hz, 2H), 3.43 (s, 3H), 3.16 (t, J=10.4 Hz, 1H), 3.01-2.80 (m, 3H), 2.73 (dd, J=11.9, 2.9 Hz, 1H), 2.68-2.58 (m, 2H), 2.57-2.53 (m, 2H), 2.47 (t, J=11.0 Hz, 1H), 2.30 (t, J=11.0 Hz, 1H), 2.17 (s, 1H), 1.94 (t, J=10.4 Hz, 1H), 1.77 (d, J=12.8 Hz, 1H), 1.63 (s, 3H), 1.59 (s, 3H), 0.93 (d, J=6.3 Hz, 3H), 0.90 (d, J=5.8 Hz, 3H).

Example 199: 4-(4-fluorobenzyl)-N,N,8,8-tetramethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxamide (compound 251)

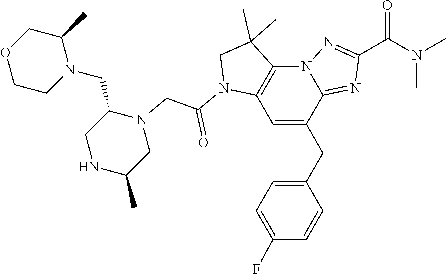 [SMILES: C[C@@H]1CN(CC(=O)N2CC(C)(C)c3c2cc(Cc2ccc(F)cc2)c2nc(C(=O)N(C)C)nn32)[C@@H](CN2CCOC[C@H]2C)CN1]

Following the synthesis of compound 31, compound 251 was obtained as a white solid. MS (ESI) m/z 621.6 [M+H]⁺. ¹H NMR (400 MHz, DMSO-d⁶) δ (ppm) 8.46 (s, 1H), 7.42 (dd, J=8.4, 5.6 Hz, 2H), 7.15 (t, J=8.7 Hz, 2H), 4.33 (d, J=2.9 Hz, 2H), 4.19 (d, J=10.6 Hz, 1H), 3.98 (d, J=10.6 Hz, 1H), 3.85 (d, J=16.7 Hz, 1H), 3.69 (d, J=16.7 Hz, 1H), 3.53-3.46 (m, 2H), 3.17 (t, J=10.3 Hz, 1H), 3.10 (s, 3H), 3.09 (s, 3H), 3.05-2.81 (m, 4H), 2.73 (dd, J=11.8, 2.8 Hz, 1H), 2.76-2.68 (m, 2H), 2.55-2.53 (m, 1H), 2.48 (t, J=10.9 Hz, 1H), 2.31 (t, J=11.0 Hz, 1H), 2.17 (s, 1H), 2.00-1.90 (m, 1H), 1.78 (d, J=12.7 Hz, 1H), 1.63 (s, 3H), 1.59 (s, 3H), 0.93 (d, J=6.2 Hz, 3H), 0.90 (d, J=5.9 Hz, 3H).

Example 200: tert-butyl (2R,5S)-4-(2-(4-(4-fluorobenzyl)-8,8-dimethyl-2-(methyl-d3)-1-oxo-1,2,7,8-tetrahydro-6H-pyrrolo[2,3-e][1,2,4]triazolo[4,3-a]pyridin-6-yl)-2-oxoethyl)-2-methyl-5-(((R)-3-methylmorpholino)methyl)piperazine-1-carboxylate (compound 252)

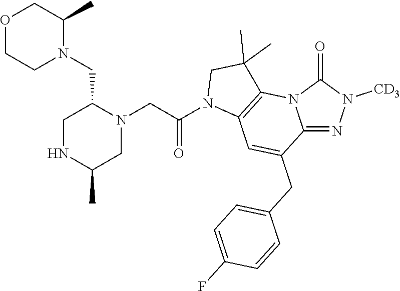 [SMILES: [2H]C([2H])([2H])n1nc2c(Cc3ccc(F)cc3)cc3c(n2c1=O)C(C)(C)CN3C(=O)CN1C[C@@H](C)NC[C@@H]1CN1CCOC[C@H]1C]

Following the synthesis of compound 135, compound 252 was obtained as a white solid (18.2 mg). ¹H NMR (400 MHz, DMSO-d⁶) δ (ppm) 7.98 (s, 1H), 7.34 (dd, J=8.5, 5.7 Hz, 2H), 7.13 (t, J=8.9 Hz, 2H), 4.03-3.90 (m, 3H), 3.78 (d, J=10.7 Hz, 1H), 3.72 (d, J=16.6 Hz, 1H), 3.56 (d, J=16.7 Hz, 1H), 3.51-3.48 (m, 2H), 3.18 (t, J=10.3 Hz, 1H), 2.99-2.86 (m, 2H), 2.86-2.74 (m, 2H), 2.69 (d, J=9.7 Hz, 1H), 2.58 (d, J=8.9 Hz, 2H), 2.42 (t, J=11.0 Hz, 1H), 2.26 (t, J=11.1 Hz, 1H), 2.20-2.09 (m, 1H), 1.90 (t, J=9.6 Hz, 1H), 1.73 (d, J=12.2 Hz, 1H), 1.57 (d, J=13.0 Hz, 6H), 0.91-0.85 (m, 6H). MS (ESI) m/z 583.6 [M+H]⁺.

Example 201: 4-(4-fluorobenzyl)-8,8-dimethyl-6-(2-((2R,5R)-5-methyl-2-(((R)-3-methylmorpholino)methyl)piperazin-1-yl)acetyl)-2-(pyridazin-3-ylmethyl)-2,6,7,8-tetrahydro-1H-pyrrolo[2,3-e][1,2,4]triazolo[4,3-a]pyridin-1-one (compound 253)

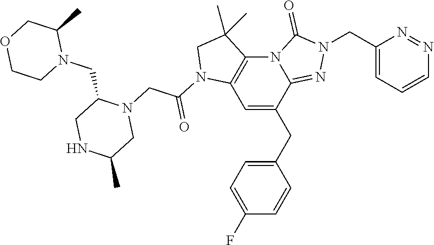 [SMILES: C[C@@H]1CN(CC(=O)N2CC(C)(C)c3c2cc(Cc2ccc(F)cc2)c2nn(Cc4cccnn4)c(=O)n32)[C@@H](CN2CCOC[C@H]2C)CN1]

Following the synthesis of compound 185, compound 253 was obtained as a white solid (11 mg). MS (ESI) m/z 657.5 [M+H]⁺.

Example 202: 4-(4-fluorobenzyl)-8,8-dimethyl-6-(2-((2R,5R)-5-methyl-2-(((R)-3-methylmorpholino)methyl)piperazin-1-yl)acetyl)-2-(pyridin-4-ylmethyl)-2,6,7,8-tetrahydro-1H-pyrrolo[2,3-e][1,2,4]triazolo[4,3-a]pyridin-1-one (compound 254)

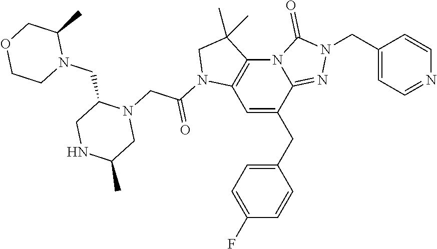 [SMILES: C[C@@H]1CN(CC(=O)N2CC(C)(C)c3c2cc(Cc2ccc(F)cc2)c2nn(Cc4ccncc4)c(=O)n32)[C@@H](CN2CCOC[C@H]2C)CN1]

Following the synthesis of compound 185, compound 254 was obtained as a white solid (35 mg). ¹H NMR (400 MHz, DMSO-d⁶) δ (ppm) 8.55 (d, J=5.8 Hz, 2H), 8.01 (s, 1H), 7.32 (dd, J=8.4, 5.7 Hz, 2H), 7.28 (d, J=5.6 Hz, 2H), 7.11 (t, J=8.8 Hz, 2H), 5.18 (s, 2H), 4.02-3.93 (m, 3H), 3.79 (d, J=10.6 Hz, 1H), 3.73 (d, J=16.6 Hz, 1H), 3.59 (d, J=16.6 Hz, 1H), 3.50 (d, J=11.1 Hz, 2H), 3.23-3.16 (m, 1H), 2.99-2.88 (m, 2H), 2.84-2.76 (m, 2H), 2.71 (d, J=10.1 Hz, 1H), 2.59 (d, J=10.7 Hz, 2H), 2.47-2.40 (m, 1H), 2.31-2.24 (m, 1H), 2.21-2.08 (m, 1H), 1.97-1.87 (m, 1H), 1.74 (d, J=12.2 Hz, 1H), 1.58 (d, J=12.4 Hz, 6H), 0.89 (t, J=6.0 Hz, 6H). MS (ESI) m/z 657.5 [M+H]⁺.

Example 203: 4-(4-fluorobenzyl)-N,8,8-trimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxamide (compound 255)

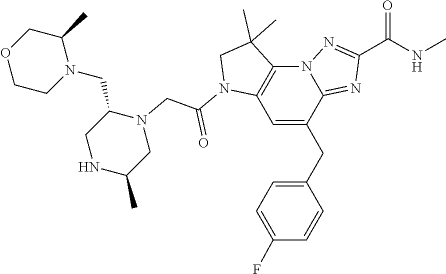 [SMILES: CNC(=O)c1nc2c(Cc3ccc(F)cc3)cc3c(n2n1)C(C)(C)CN3C(=O)CN1C[C@@H](C)NC[C@@H]1CN1CCOC[C@H]1C]

Following the synthesis of compound 31, compound 255 was obtained as a white solid. MS (ESI) m/z 607.5 [M+H]⁺. ¹H NMR (400 MHz, DMSO-d⁶) δ (ppm) 8.70 (q, J=4.7 Hz, 1H), 8.44 (s, 1H), 7.45 (dd, J=8.3, 5.5 Hz, 2H), 7.15 (t, J=8.7 Hz, 2H), 4.42-4.29 (m, 2H), 4.19 (d, J=10.5 Hz, 1H), 3.98 (d, J=10.5 Hz, 1H), 3.87 (d, J=16.6 Hz, 1H), 3.73-3.62 (m, 1H), 3.54-3.44 (m, 2H), 3.17 (t, J=10.3 Hz, 1H), 3.03-2.90 (m, 2H), 2.88 (d, J=4.8 Hz, 3H), 2.83 (d, J=11.9 Hz, 1H), 2.78-2.70 (m, 1H), 2.64 (d, J=10.0 Hz, 2H), 2.57-2.53 (m, 2H), 2.46 (t, J=11.0 Hz, 1H), 2.31 (t, J=11.0 Hz, 1H), 2.17 (s, 1H), 1.94 (t, J=10.0 Hz, 1H), 1.78 (d, J=12.8 Hz, 1H), 1.66 (s, 3H), 1.62 (s, 3H), 0.92 (dd, J=8.9, 6.0 Hz, 6H).

Example 204: 4-(4-fluorobenzyl)-N-isopropyl-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxamide (compound 256)

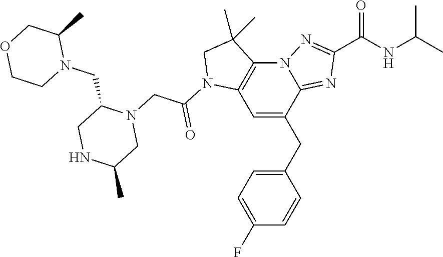 [SMILES: CC(C)NC(=O)c1nc2c(Cc3ccc(F)cc3)cc3c(n2n1)C(C)(C)CN3C(=O)CN1C[C@@H](C)NC[C@@H]1CN1CCOC[C@H]1C]

Following the synthesis of compound 31, compound 256 was obtained as a white solid. MS (ESI) m/z 635.6 [M+H]⁺. ¹H NMR (400 MHz, DMSO-d⁶) δ (ppm) 8.44-8.37 (m, 2H), 7.46-7.38 (m, 2H), 7.21-7.11 (m, 2H), 4.45-4.31 (m, 2H), 4.21-4.17 (m, 1H), 3.99 (d, J=10.5 Hz, 1H), 3.88 (d, J=16.6 Hz, 1H), 3.64 (d, J=16.6 Hz, 1H), 3.49 (t, J=8.5 Hz, 2H), 3.17 (t, J=10.4 Hz, 1H), 2.97-2.79 (m, 4H), 2.73 (dd, J=11.7, 2.7 Hz, 1H), 2.63 (d, J=9.1 Hz, 2H), 2.57-2.53 (m, 1H), 2.43 (t, J=11.0 Hz, 1H), 2.31 (t, J=11.0 Hz, 1H), 2.17 (s, 1H), 2.03 (s, 1H), 1.95 (t, J=10.6 Hz, 1H), 1.78 (d, J=12.6 Hz, 1H), 1.66 (s, 3H), 1.62 (s, 3H), 1.26 (s, 3H), 1.25 (s, 3H), 0.93 (d, J=6.2 Hz, 3H), 0.90 (d, J=6.0 Hz, 3H).

Example 205: N-cyclopropyl-4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxamide (compound 257)

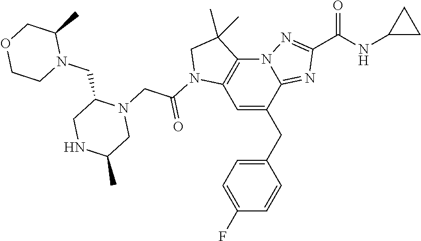 [SMILES: C[C@@H]1CN(CC(=O)N2CC(C)(C)c3c2cc(Cc2ccc(F)cc2)c2nc(C(=O)NC4CC4)nn32)[C@@H](CN2CCOC[C@H]2C)CN1]

Following the synthesis of compound 31, compound 257 was obtained as a white solid. MS (ESI) m/z 633.5 [M+H]⁺. ¹H NMR (400 MHz, DMSO-d⁶) δ (ppm) 8.77 (dd, J=4.6, 2.3 Hz, 1H), 8.46 (d, J=2.5 Hz, 1H), 7.46-7.37 (m, 2H), 7.20-7.14 (m, 2H), 4.47-4.34 (m, 2H), 4.24 (dd, J=10.5, 2.5 Hz, 1H), 4.03 (dd, J=10.5, 2.5 Hz, 1H), 3.92 (dd, J=16.7, 2.6 Hz, 1H), 3.69 (d, J=16.6 Hz, 1H), 3.52 (d, J=8.4 Hz, 2H), 3.21 (t, J=10.5 Hz, 1H), 3.05-2.85 (m, 4H), 2.78 (d, J=11.7 Hz, 1H), 2.68 (d, J=11.2 Hz, 2H), 2.61-2.58 (m, 2H), 2.48 (t, J=10.4 Hz, 1H), 2.35 (t, J=11.0 Hz, 1H), 2.21 (s, 1H), 1.99 (t, J=10.7 Hz, 1H), 1.82 (d, J=12.8 Hz, 1H), 1.70 (d, J=2.5 Hz, 3H), 1.66 (d, J=2.4 Hz, 3H), 1.00-0.91 (m, 6H), 0.87-0.74 (m, 4H).

Example 206: 4-(4-fluorobenzyl)-8,8-dimethyl-N-(methyl-d3)-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxamide (compound 258)

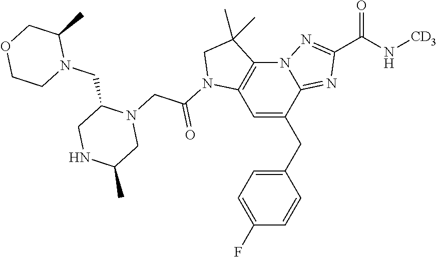 [SMILES: [2H]C([2H])([2H])NC(=O)c1nc2c(Cc3ccc(F)cc3)cc3c(n2n1)C(C)(C)CN3C(=O)CN1C[C@@H](C)NC[C@@H]1CN1CCOC[C@H]1C]

Following the synthesis of compound 31, compound 258 was obtained as a white solid. MS (ESI) m/z 610.6 [M+H]⁺. ¹H NMR (400 MHz, DMSO-d⁶) δ (ppm) 8.68 (s, 1H), 8.44 (s, 1H), 7.51-7.40 (m, 2H), 7.16 (t, J=8.7 Hz, 2H), 4.42-4.30 (m, 2H), 4.20 (d, J=10.6 Hz, 1H), 3.99 (d, J=10.5 Hz, 1H), 3.88 (d, J=16.6 Hz, 1H), 3.66 (d, J=16.6 Hz, 1H), 3.55-3.45 (m, 2H), 3.17 (t, J=10.3 Hz, 1H), 3.03-2.80 (m, 4H), 2.74 (dd, J=11.9, 2.8 Hz, 1H), 2.64 (d, J=9.3 Hz, 2H), 2.58-2.55 (m, 1H), 2.45 (t, J=11.0 Hz, 1H), 2.31 (t, J=11.0 Hz, 1H), 2.17 (s, 1H), 1.95 (t, J=10.3 Hz, 1H), 1.78 (d, J=12.8 Hz, 1H), 1.66 (s, 3H), 1.62 (s, 3H), 0.93 (d, J=6.2 Hz, 3H), 0.90 (d, J=6.0 Hz, 3H).

Example 207: N-ethyl-4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridine-2-carboxamide (compound 259)

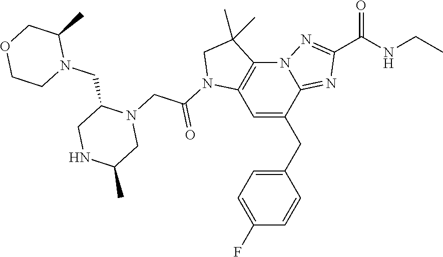 [SMILES: CCNC(=O)c1nc2c(Cc3ccc(F)cc3)cc3c(n2n1)C(C)(C)CN3C(=O)CN1C[C@@H](C)NC[C@@H]1CN1CCOC[C@H]1C]

Following the synthesis of compound 31, compound 259 was obtained as a white solid. MS (ESI) m/z 621.5 [M+H]⁺. ¹H NMR (400 MHz, DMSO-d⁶) δ (ppm) 8.77 (t, J=6.0 Hz, 1H), 8.42 (s, 1H), 7.49-7.41 (m, 2H), 7.21-7.11 (m, 2H), 4.43-4.30 (m, 2H), 4.20 (d, J=10.6 Hz, 1H), 3.99 (d, J=10.6 Hz, 1H), 3.88 (d, J=16.6 Hz, 1H), 3.66 (d, J=16.6 Hz, 1H), 3.54-3.45 (m, 2H), 3.43-3.49 (m, 2H), 3.17 (t, J=10.2 Hz, 1H), 3.03-2.80 (m, 4H), 2.75 (dd, J=11.7, 2.6 Hz, 1H), 2.68-2.58 (m, 2H), 2.56-2.54 (m, 1H), 2.46 (t, J=11.0 Hz, 1H), 2.32 (t, J=11.0 Hz, 1H), 2.18 (s, 1H), 1.95 (t, J=10.4 Hz, 1H), 1.79 (d, J=12.6 Hz, 1H), 1.67 (s, 3H), 1.62 (s, 3H), 1.19 (t, J=7.1 Hz, 3H), 0.93 (d, J=6.3 Hz, 3H), 0.91 (d, J=6.0 Hz, 3H).

Example 208: 4-(4-fluorobenzyl)-2-(1-hydroxy-2-methylpropan-2-yl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 260)

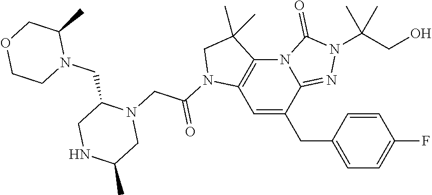 [SMILES: C[C@@H]1CN(CC(=O)N2CC(C)(C)c3c2cc(Cc2ccc(F)cc2)c2nn(C(C)(C)CO)c(=O)n32)[C@@H](CN2CCOC[C@H]2C)CN1]

Following the synthesis of compound 275, compound 260 was obtained as a yellow solid (21.7 mg)

¹H NMR (400 MHz, DMSO-d⁶) δ (ppm) 8.00 (s, 1H), 7.40 (dd, J=8.1, 5.8 Hz, 2H), 7.14 (t, J=8.8 Hz, 2H), 4.82 (t, J=6.1 Hz, 1H), 4.04-3.89 (m, 3H), 3.83-3.71 (m, 3H), 3.70-3.59 (m, 2H), 3.58-3.46 (m, 2H), 3.23-3.14 (m, 1H), 3.05-2.56 (m, 8H), 2.50-2.45 (m, 1H), 2.32-2.24 (m, 1H), 2.20-2.11 (m, 1H), 1.96-1.87 (m, 1H), 1.80-1.71 (m, 1H), 1.57 (d, J=13.1 Hz, 6H), 1.54 (s, 6H), 0.94-0.87 (m, 6H). MS (ESI) m/z 638.55 [M+H]⁺

Example 209: 2-(1,3-dimethoxypropan-2-yl)-4-(4-fluorobenzyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 261)

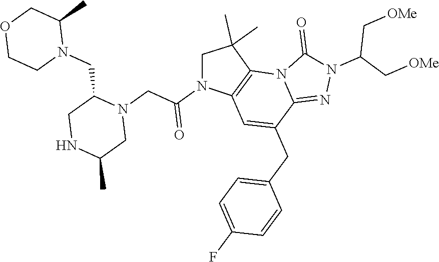 [SMILES: COCC(COC)n1nc2c(Cc3ccc(F)cc3)cc3c(n2c1=O)C(C)(C)CN3C(=O)CN1C[C@@H](C)NC[C@@H]1CN1CCOC[C@H]1C]

Following the synthesis of compound 228, compound 261 was obtained as a white solid (31.9 mg). MS (ESI) m/z 668.9[M+H]⁺. ¹H NMR (400 MHz, DMSO-d⁶) δ (ppm) 8.05 (s, 1H), 7.46-7.33 (m, 2H), 7.19-7.08 (m, 2H), 4.75-4.61 (m, 1H), 4.07-3.92 (m, 3H), 3.79 (d, J=10.6 Hz, 1H), 3.77-3.68 (m, 3H), 3.64-3.56 (m, 3H), 3.56-3.45 (m, 2H), 3.26-3.17 (m, 7H), 3.01-2.89 (m, 2H), 2.88-2.78 (m, 2H), 2.74-2.65 (m, 1H), 2.65-2.55 (m, 2H), 2.49-2.40 (m, 1H), 2.32-2.22 (m, 1H), 2.21-2.10 (m, 1H), 2.03-1.89 (m, 1H), 1.74 (d, J=12.3 Hz, 1H), 1.58 (d, J=11.4 Hz, 6H), 0.93-0.86 (m, 6H).

Example 210: 4-(4-fluorobenzyl)-2-((1-hydroxycyclobutyl)methyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 262)

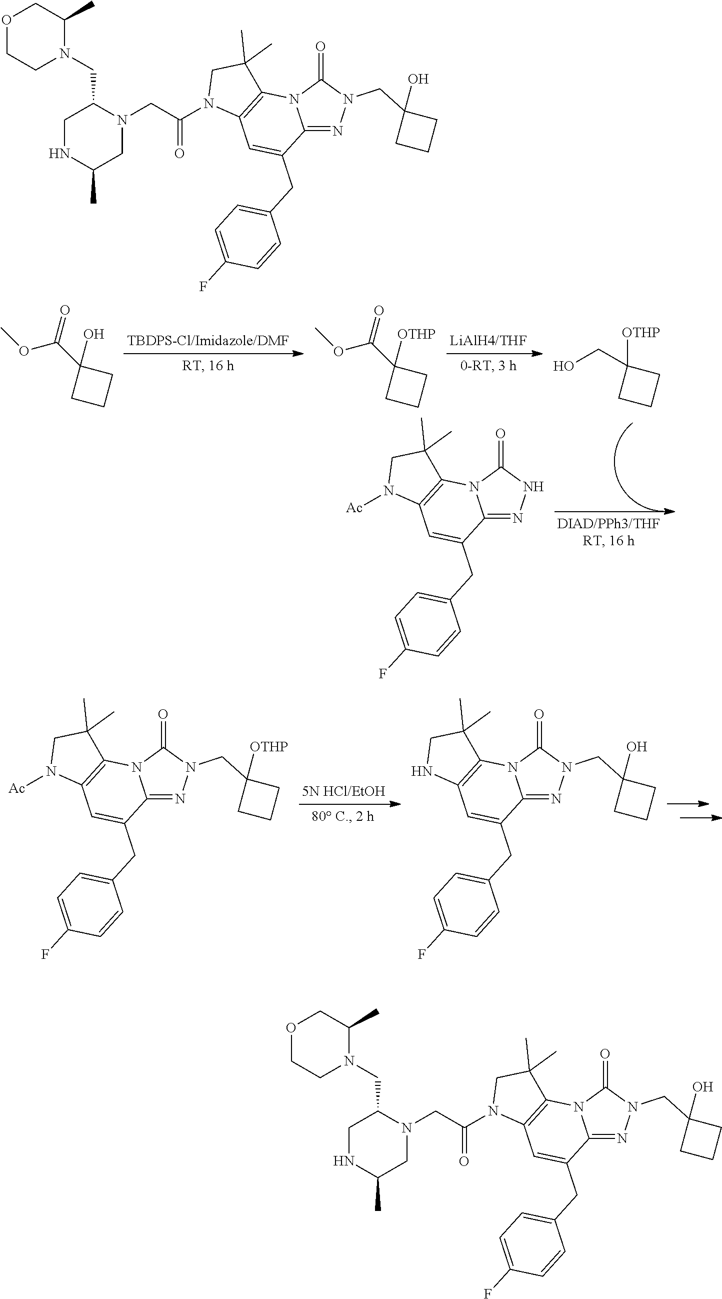

Step 1: methyl 1-((tetrahydro-2H-pyran-2-yl)oxy)cyclobutane-1-carboxylate

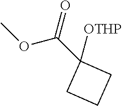

Methyl 1-hydroxycyclobutane-1-carboxylate (1 g, 7.68 mmol, 1.0 eq), PPTS (0.193 g, 0.768 mmol, 0.1 eq), and DHP (0.776 g, 9.22 mmol, 1.2 eq) were dissolved in DCM (20 mL) under argon to give a colorless solution. The reaction mixture was stirred at rt for 16 h. H₂O (20 mL) was added to the reaction mixture followed by extraction with dichloromethane (30 mL×2). The combined organic layers were dried over Na₂SO₄, filtered and concentrated. The crude product was added to a silica gel column and was eluted with ethyl acetate/hexane from 0% to 20% to give the product (1.6 g, 97%) as a colorless liquid. MS (ESI) m/z 215.4 [M+H⁺.

Step 2: (1-((tetrahydro-2H-pyran-2-yl)oxy)cyclobutyl)methanol

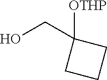

Methyl 1-((tetrahydro-2H-pyran-2-yl)oxy)cyclobutane-1-carboxylate (1.6 g, 7.47 mmol, 1.0 eq) was dissolved in THF (20 mL) under argon to give a solution. The reaction mixture was cooled to 0° C. with an ice/water bath. LiAlH₄ (0.425 g, 11.20 mmol, 1.5 eq) was added to the reaction mixture in one portion. The reaction mixture was stirred at rt for 3 h. H₂O (1 mL) was added to the reaction mixture followed by extraction with ethyl acetate (50 mL×2). The combined organic layers were dried over Na₂SO₄, filtered and concentrated. The crude product was added to a silica gel column and was eluted with ethyl acetate/hexane from 0% to 30% to give the product (1.35 g, 97%) as a colorless liquid. MS (ESI) m/z 187.3 [M+H⁺.

Step 3: 6-acetyl-4-(4-fluorobenzyl)-8,8-dimethyl-2-((1-((tetrahydro-2H-pyran-2-yl)oxy)cyclo butyl)methyl)-2,6,7,8-tetrahydro-1H-pyrrolo[2,3-e][1,2,4]triazolo[4,3-a]pyridin-1-one

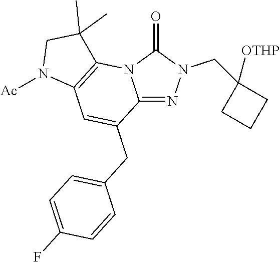

6-Acetyl-4-(4-fluorobenzyl)-8,8-dimethyl-2,6,7,8-tetrahydro-1H-pyrrolo[2,3-e][1,2,4]triazolo[4,3-a]pyridin-1-one (0.7 g, 1.975 mmol, 1.0 eq), (1-((tetrahydro-2H-pyran-2-yl)oxy)cyclobutyl)methanol (0.736 g, 3.95 mmol, 2.0 eq), triphenylphosphine (0.777 g, 2.96 mmol, 1.5 eq), and DIAD (0.799 g, 3.95 mmol, 2.0 eq) were dissolved in THF (20 mL) under argon to give a yellow solution. The reaction mixture was stirred at rt for 16 h. Saturated NaCl (20 mL) was added to the reaction mixture followed by extraction with ethyl acetate (30 mL×2). The combined organic layers were dried over Na₂SO₄, filtered and concentrated. The crude product was added to a silica gel column and was eluted with ethyl acetate/hexane from 0% to 80% to give the product (1.19 g, 100%) as a yellow oil. MS (ESI) m/z 523.4 [M+H]⁺.

Following Steps: following the synthesis of compound 14, compound 262 was synthesized as a white solid (0.7 g). MS (ESI) m/z 650.6 [M+H]⁺.

Example 211: 4-(4-fluorobenzyl)-8,8-dimethyl-6-(2-((2R,5R)-5-methyl-2-(((R)-3-methylmorpholino)methyl)piperazin-1-yl)acetyl)-2-(2,2,2-trifluoroethyl)-2,6,7,8-tetrahydro-1H-pyrrolo[2,3-e][1,2,4]triazolo[4,3-a]pyridin-1-one (compound 263)

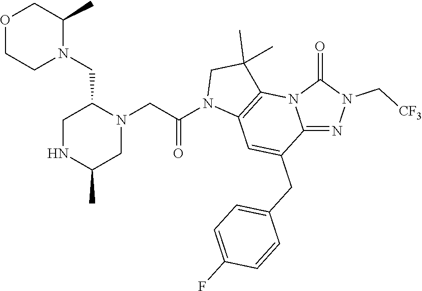 [SMILES: C[C@@H]1CN(CC(=O)N2CC(C)(C)c3c2cc(Cc2ccc(F)cc2)c2nn(CC(F)(F)F)c(=O)n32)[C@@H](CN2CCOC[C@H]2C)CN1]

Following the synthesis of compound 185, compound 263 was obtained as a white solid (19.5 mg). MS (ESI) m/z 648.4 [M+H]⁺.

Example 212: 2-(2,2-difluoroethyl)-4-(4-fluorobenzyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 264)

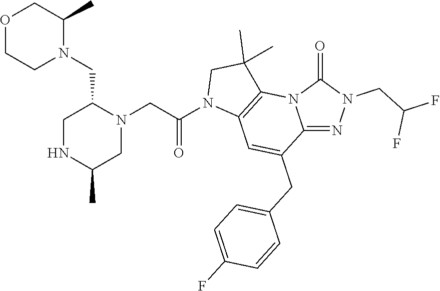 [SMILES: C[C@@H]1CN(CC(=O)N2CC(C)(C)c3c2cc(Cc2ccc(F)cc2)c2nn(CC(F)F)c(=O)n32)[C@@H](CN2CCOC[C@H]2C)CN1]

Following the synthesis of compound 185, compound 264 was obtained as a white solid (19.5 mg). MS (ESI) m/z 629.5 [M+H]⁺.

Example 213: 6-(2-((2R,5R)-2-((8-oxa-3-azabicyclo[3.2.1]octan-3-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one (compound 265)

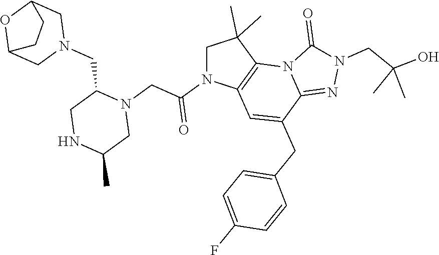 [SMILES: C[C@@H]1CN(CC(=O)N2CC(C)(C)c3c2cc(Cc2ccc(F)cc2)c2nn(CC(C)(C)O)c(=O)n32)[C@@H](CN2CC3CCC(C2)O3)CN1]

Following the synthesis of compound 274, compound 265 was obtained as a white solid (74.1 mg). MS (ESI) m/z 650.6 [M+H]⁺.

Example 214: 4-(4-fluorobenzyl)-2-(2-hydroxy-2-methylpropyl)-8,8-dim ethyl-6-(2-((2R,5R)-5-methyl-2-(((R)-2-methyl-4-(2,2,2-trifluoroethyl)piperazin-1-yl)methyl)piperazin-1-yl)acetyl)-2,6,7,8-tetrahydro-1H-pyrrolo[2,3-e][1,2,4]triazolo[4,3-a]pyridin-1-one (compound 266)

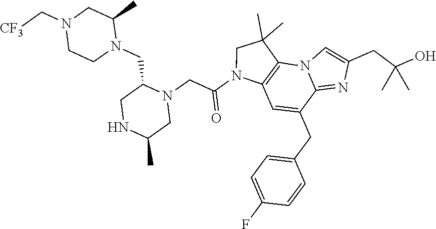 [SMILES: C[C@@H]1CN(CC(=O)N2CC(C)(C)c3c2cc(Cc2ccc(F)cc2)c2nc(CC(C)(C)O)cn32)[C@@H](CN2CCN(CC(F)(F)F)C[C@H]2C)CN1]

Following the synthesis of compound 274, compound 266 was obtained as a white solid (144.2 mg). MS (ESI) m/z 719.4 [M+H]⁺.

Example 215: 4-(4-fluorobenzyl)-2-(2-hydroxy-2-methylpropyl)-8,8-dimethyl-6-(2-((2R,5R)-5-methyl-2-(((R)-2-methyl-4-(oxetan-3-yl)piperazin-1-yl)methyl)piperazin-1-yl)acetyl)-2,6,7,8-tetrahydro-1H-pyrrolo[2,3-e][1,2,4]triazolo[4,3-a]pyridin-1-one (compound 267)

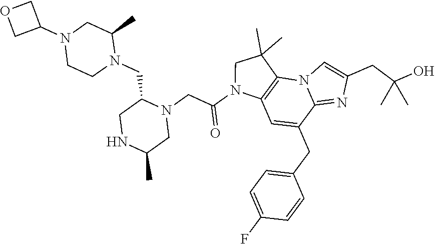 [SMILES: C[C@@H]1CN(CC(=O)N2CC(C)(C)c3c2cc(Cc2ccc(F)cc2)c2nc(CC(C)(C)O)cn32)[C@@H](CN2CCN(C3COC3)C[C@H]2C)CN1]

Following the synthesis of compound 274, compound 267 was obtained as a white solid (110.5 mg). MS (ESI) m/z 693.6 [M+H]⁺.

Example 216: 2-(1,3-dihydroxypropan-2-yl)-4-(4-fluorobenzyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 268)

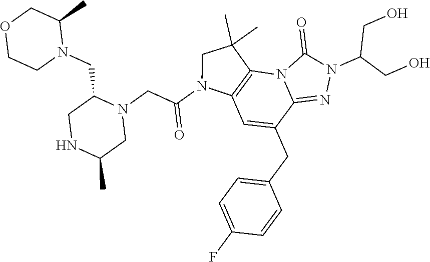 [SMILES: C[C@@H]1CN(CC(=O)N2CC(C)(C)c3c2cc(Cc2ccc(F)cc2)c2nn(C(CO)CO)c(=O)n32)[C@@H](CN2CCOC[C@H]2C)CN1]

Following the synthesis of compound 274, compound 268 was obtained as a white solid (107 mg).

MS (ESI) m/z 640.4[M+H]+. ¹H NMR (400 MHz, DMSO-d⁶) δ (ppm) 7.95 (s, 1H), 7.49-7.33 (m, 2H), 7.20-7.08 (m, 2H), 4.90-4.78 (m, 2H), 4.48-4.29 (m, 1H), 4.03-3.91 (m, 3H), 3.78 (d, J=10.6 Hz, 1H), 3.74-3.65 (m, 5H), 3.57-3.46 (m, 2H), 3.22-3.05 (m, 1H), 2.96-2.76 (m, 3H), 2.75-2.56 (m, 2H), 2.56-2.52 (m, 1H), 2.41-2.27 (m, 1H), 2.23-2.11 (m, 1H), 2.09-1.87 (m, 2H), 1.78 (d, 1H), 1.60 (d, J=12.7 Hz, 6H), 0.98-0.86 (m, 6H).

Example 217: 4-(4-fluorobenzyl)-2-(2-hydroxypropyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 269)

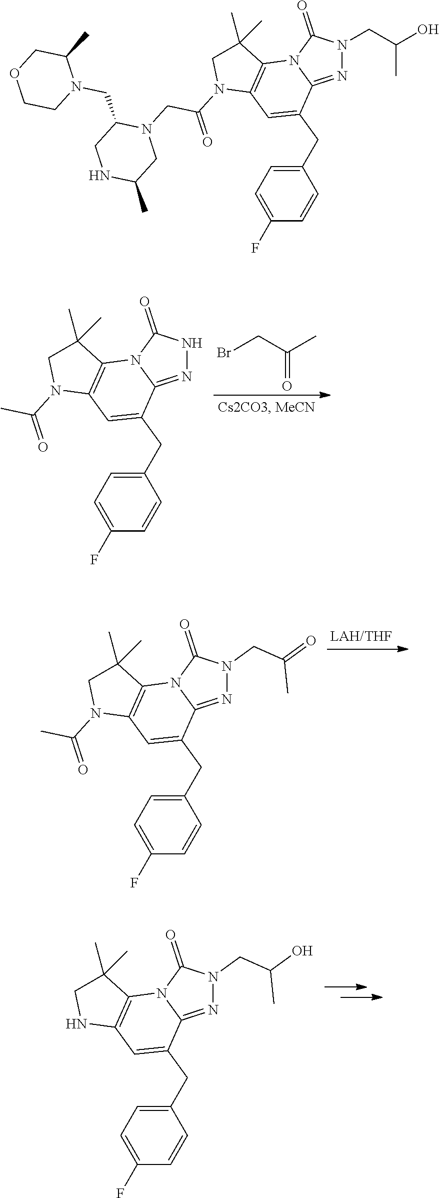

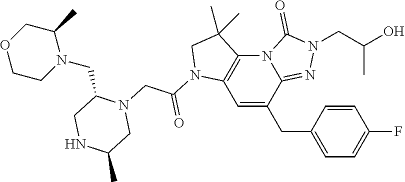 [SMILES: CC(O)Cn1nc2c(Cc3ccc(F)cc3)cc3c(n2c1=O)C(C)(C)CN3C(=O)CN1C[C@@H](C)NC[C@@H]1CN1CCOC[C@H]1C]

Step 1: 6-acetyl-4-(4-fluorobenzyl)-8,8-dimethyl-2-(2-oxopropyl)-2,6,7,8-tetrahydro-1H-pyrrolo[2,3-e][1,2,4]triazolo[4,3-a]pyridin-1-one

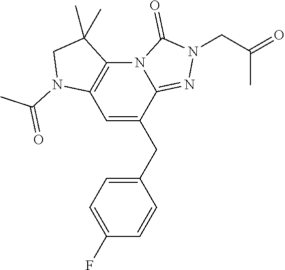 [SMILES: CC(=O)Cn1nc2c(Cc3ccc(F)cc3)cc3c(n2c1=O)C(C)(C)CN3C(C)=O]

A mixture of 6-acetyl-4-(4-fluorobenzyl)-8,8-dimethyl-2,6,7,8-tetrahydro-1H-pyrrolo[2,3-e][1,2,4]triazolo[4,3-a]pyridin-1-one (360 mg, 1.016 mmol, 1.0 eq), 1-bromopropan-2-one (209 mg, 1.524 mmol, 1.5 eq) and cesium carbonate (662 mg, 2.032 mmol, 2.0 eq) in DMF (10 mL) was stirred at 50-60° C. for 2 h. The mixture was diluted with water, extracted with ethyl acetate (twice). The organic layer was washed with brine, dried over Na₂SO₄ and concentrated to give the title compound (380 mg, 91%) as a yellow oil. MS (ESI) m/z 411.77 [M+H]⁺

Step 2: 4-(4-fluorobenzyl)-2-(2-hydroxypropyl)-8,8-dimethyl-2,6,7,8-tetrahydro-1H-pyrrolo[2,3-e][1,2,4]triazolo[4,3-a]pyridin-1-one

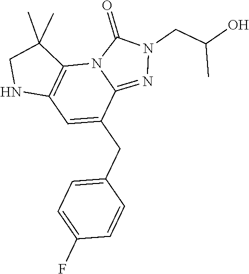 [SMILES: CC(O)Cn1nc2c(Cc3ccc(F)cc3)cc3c(n2c1=O)C(C)(C)CN3]

To a solution of 6-acetyl-4-(4-fluorobenzyl)-8,8-dimethyl-2-(2-oxopropyl)-2,6,7,8-tetrahydro-1H-pyrrolo[2,3-e][1,2,4]triazolo[4,3-a]pyridin-1-one (380 mg, 0.926 mmol, 1.0 eq) in THF (6 mL) was added LAH (70.3 mg, 1.852 mmol, 2.0 eq) at 0-10° C. The reaction mixture was stirred at 0-10° C. for 1 h. The mixture was quenched with Na₂SO₄·10H₂O. The mixture was stirred at 0-10° C. for 0.5 h. The resulting suspension was filtered, the filtrate was concentrated to give the title compound (300 mg, 87%) as a yellow oil. MS (ESI) m/z 371.71 [M+H]⁺

Following steps: following the synthesis of compound 14, compound 269 was synthesized as a white solid (92 mg, 26.7%). ¹H NMR (400 MHz, DMSO-d⁶) δ (ppm) 8.01 (s, 1H), 7.42-7.35 (m, 2H), 7.20-7.12 (m, 2H), 4.93-4.84 (m, 1H), 4.14-3.69 (m, 8H), 3.66-3.48 (m, 3H), 3.27-3.16 (m, 2H), 3.01-2.78 (m, 4H), 2.65-2.57 (m, 3H), 2.50-2.41 (m, 1H), 2.34-2.24 (m, 1H), 2.22-2.12 (m, 11H), 1.99-1.88 (m, 1H), 1.81-1.71 (m, 11H), 1.60 (d, J=13.1 Hz, 6H), 1.15 (d, J=6.3 Hz, 3H), 0.98-0.85 (m, 6H). MS (ESI) m/z 624.59 [M+H]⁺.

Example 218: 2-(2-cyclopropyl-2-hydroxyethyl)-4-(4-fluorobenzyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one TFA salt (Compound 270)

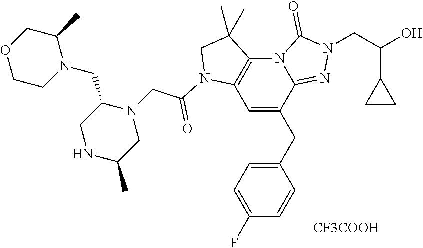

Following the synthesis of compound 269, compound 270 was obtained as a yellow solid (60 mg) after pre-HPLC purification. ¹H NMR (400 MHz, DMSO-d⁶) δ (ppm) 9.29 (s, 1H), 8.89 (s, 1H), 7.78 (s, 1H), 7.45-7.33 (m, 2H), 7.23-7.13 (m, 2H), 4.21-2.57 (m, 24H), 1.64 (d, J=3.5 Hz, 6H), 1.31-1.02 (m, 6H), 0.96-0.82 (m, 11H), 0.48-0.25 (m, 3H), 0.12-−0.02 (m, 1H). MS (ESI) m/z 650.52 [M+H]⁺

Example 219: 4-(4-fluorobenzyl)-2-(3-hydroxycyclobutyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one TFA salt (compound 271)

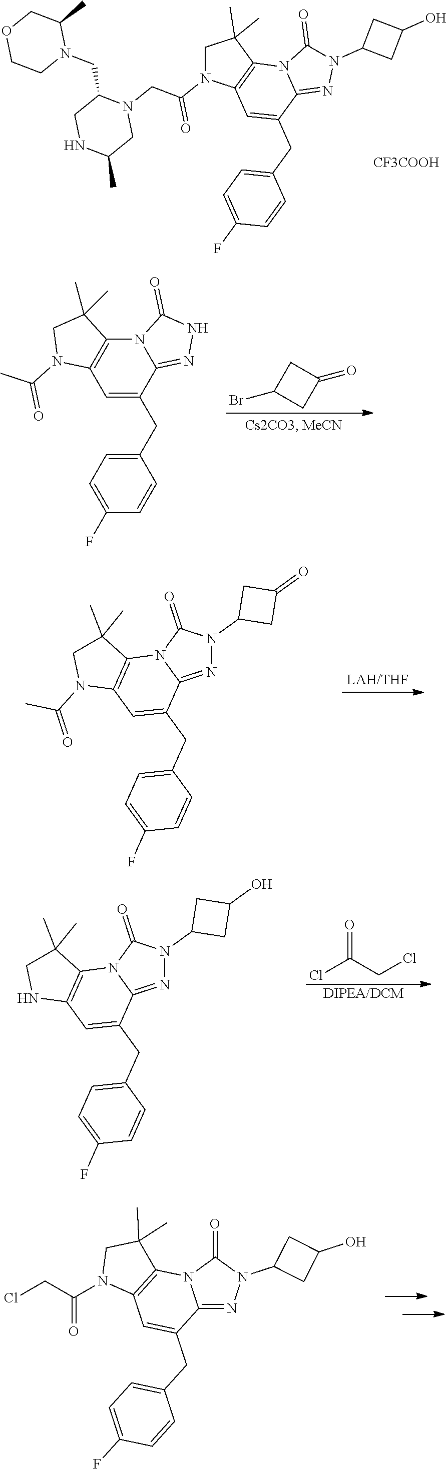

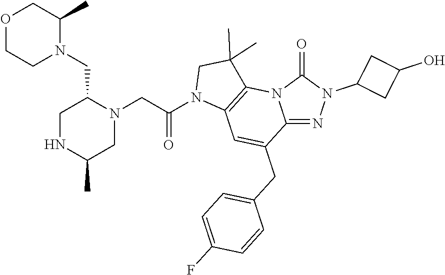 [SMILES: C[C@@H]1CN(CC(=O)N2CC(C)(C)c3c2cc(Cc2ccc(F)cc2)c2nn(C4CC(O)C4)c(=O)n32)[C@@H](CN2CCOC[C@H]2C)CN1]

Step 1: 6-acetyl-4-(4-fluorobenzyl)-8,8-dimethyl-2-(3-oxocyclobutyl)-2,6,7,8-tetrahydro-1H-pyrrolo[2,3-e][1,2,4]triazolo[4,3-a]pyridin-1-one

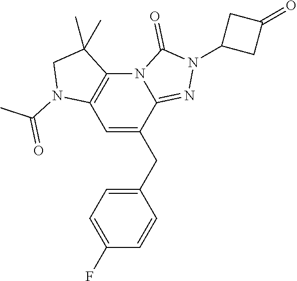 [SMILES: CC(=O)N1CC(C)(C)c2c1cc(Cc1ccc(F)cc1)c1nn(C3CC(=O)C3)c(=O)n21]

A mixture of 6-acetyl-4-(4-fluorobenzyl)-8,8-dimethyl-2,6,7,8-tetrahydro-1H-pyrrolo[2,3-e][1,2,4]triazolo[4,3-a]pyridin-1-one (600 mg, 1.693 mmol, 1.0 eq), 3-bromocyclobutan-1-one (378 mg, 2.54 mmol, 1.5 eq), Cs₂CO₃ (607 mg, 1.862 mmol, 1.1 eq) in DMF (10 mL) was stirred at 60° C. for 2 h. The mixture was diluted with water, extracted with ethyl acetate (twice). The organic layer was washed with brine, dried over Na₂SO₄ and concentrated to give a yellow oil, which was purified by column eluted with 80% EA/PE to give the title compound (520 mg, 72.7% yield) as a yellow oil. MS (ESI) m/z 423.28 [M+H]⁺

Step 2: 4-(4-fluorobenzyl)-2-(3-hydroxycyclobutyl)-8,8-dimethyl-2,6,7,8-tetrahydro-1H-pyrrolo[2,3-e]1,2,4]triazolo[4,3-a]pyridin-1-one

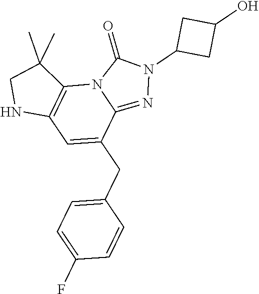 [SMILES: CC1(C)CNc2cc(Cc3ccc(F)cc3)c3nn(C4CC(O)C4)c(=O)n3c21]

To a solution of 6-acetyl-4-(4-fluorobenzyl)-8,8-dimethyl-2-(3-oxocyclobutyl)-2,6,7,8-tetrahydro-1H-pyrrolo[2,3-e][1,2,4]triazolo[4,3-a]pyridin-1-one (250 mg, 0.592 mmol, 1.0 eq) in THF (6 mL) was added LAH (44.9 mg, 1.184 mmol, 2.0 eq) at 0-10° C. The reaction mixture was stirred at 0-10° C. for 1 h. The mixture was quenched with Na₂SO₄·10H₂O, stirred at 0-10° C. for 0.5 h. The resulting suspension was filtered and the filtrate was concentrated to give the title compound (200 mg, 88%) as a yellow oil. MS (ESI) m/z 383.65 [M+H]⁺

Step 3: 6-(2-chloroacetyl)-4-(4-fluorobenzyl)-2-(3-hydroxycyclobutyl)-8,8-dimethyl-2,6,7,8-tetrahydro-1H-pyrrolo[2,3-e][1,2,4]triazolo[4,3-a]pyridin-1-one

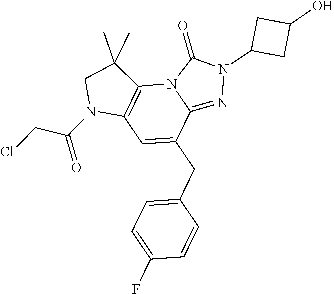 [SMILES: CC1(C)CN(C(=O)CCl)c2cc(Cc3ccc(F)cc3)c3nn(C4CC(O)C4)c(=O)n3c21]

To a mixture of 4-(4-fluorobenzyl)-2-(3-hydroxycyclobutyl)-8,8-dimethyl-2,6,7,8-tetrahydro-1H-pyrrolo[2,3-e][1,2,4]triazolo[4,3-a]pyridin-1-one (200 mg, 0.523 mmol, 1.0 eq) and N-ethyl-N-isopropylpropan-2-amine (135 mg, 1.046 mmol, 2.0 eq) in DCM (10 mL) was added 2-chloroacetyl chloride (118 mg, 1.046 mmol, 2.0 eq) at 0-10° C. The reaction mixture was stirred for 1 h. The mixture was quenched with H₂O, extracted with DCM. The organic layer was washed with brine, dried over Na₂SO₄ and concentrated to give the title compound (200 mg, 83%) as a yellow oil. MS (ESI) m/z 459.24 [M+H]⁺

Following steps: following the synthesis of compound 14, compound 271 was obtained as a yellow solid (33 mg, 21.22%). ¹H NMR (400 MHz, DMSO-d⁶) δ (ppm) 7.79 (s, 1H), 7.48-7.36 (m, 2H), 7.25-7.16 (m, 2H), 4.47-4.39 (m, 1H), 4.08-3.01 (m, 24H), 2.70-2.62 (m, 2H), 2.49-2.38 (m, 2H), 1.62 (s, 6H), 1.30-1.01 (m, 6H). MS (ESI) m/z 636.52 [M+H]⁺

Example 220: 4-(4-fluorobenzyl)-2-(3-hydroxy-3-methylcyclobutyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one TFA salt (compound 272)

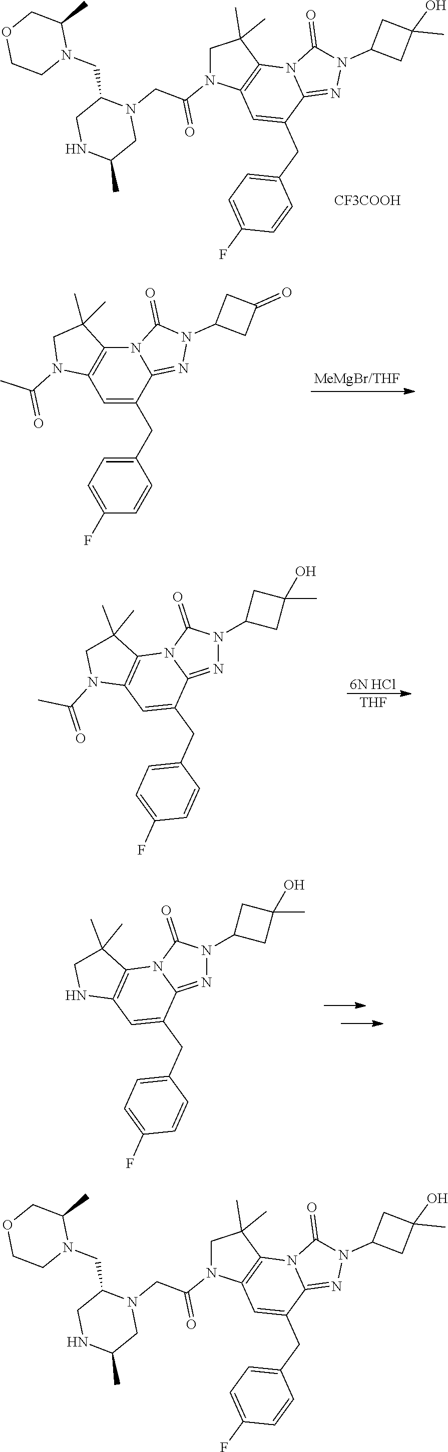

Step 1: 6-acetyl-4-(4-fluorobenzyl)-2-(3-hydroxy-3-methylcyclobutyl)-8,8-dimethyl-2,6,7,8-tetrahydro-1H-pyrrolo[2,3-e][1,2,4]triazolo[4,3-a]pyridin-1-one

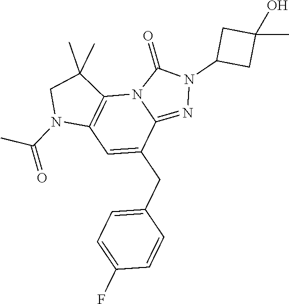 [SMILES: CC(=O)N1CC(C)(C)c2c1cc(Cc1ccc(F)cc1)c1nn(C3CC(C)(O)C3)c(=O)n21]

To a solution of 6-acetyl-4-(4-fluorobenzyl)-8,8-dimethyl-2-(3-oxocyclobutyl)-2,6,7,8-tetrahydro-1H-pyrrolo[2,3-e][1,2,4]triazolo[4,3-a]pyridin-1-one (250 mg, 0.592 mmol, 1.0 eq) in THF (10 mL) was added methylmagnesium bromide (106 mg, 0.888 mmol, 1.5 eq) at −60° C. The mixture was stirred at −60° C. for 1 h. The mixture was quenched with NH₄Cl(aq), extracted with ethyl acetate (twice). The organic layer was washed with brine, dried over Na₂SO₄ and concentrated to give the title compound (250 mg, 96%) as a yellow oil. MS (ESI) m/z 439.35 [M+H]⁺

Step 2: 4-(4-fluorobenzyl)-2-(3-hydroxy-3-methylcyclobutyl)-8,8-dimethyl-2,6,7,8-tetrahydro-1H-pyrrolo[2,3-e][1,2,4]triazolo[4,3-a]pyridin-1-one

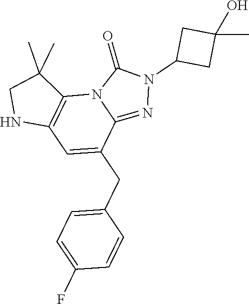 [SMILES: CC1(O)CC(n2nc3c(Cc4ccc(F)cc4)cc4c(n3c2=O)C(C)(C)CN4)C1]

A mixture of 6-acetyl-4-(4-fluorobenzyl)-2-(3-hydroxy-3-methylcyclobutyl)-8,8-dimethyl-2,6,7,8-tetrahydro-1H-pyrrolo[2,3-e][1,2,4]triazolo[4,3-a]pyridin-1-one (250 mg, 0.570 mmol, 1.0 eq) in THF (5 mL)/6N HCl (5.00 mL) was stirred at 80° C. for 2 h. The mixture was concentrated to give the title compound (crude, HCl salt) as a yellow oil, which was used into next step directly. MS (ESI) m/z 397.34 [M+H]⁺

Following steps: following the synthesis of compound 14, compound 272 was obtained as a yellow solid (32 mg, 23.08%). ¹H NMR (400 MHz, DMSO-d⁶) δ (ppm) 9.30 (s, 1H), 8.92 (s, 1H), 7.78 (s, 1H), 7.46-7.38 (m, 2H), 7.24-7.15 (m, 2H), 4.59-4.46 (m, 1H), 4.12-3.97 (m, 3H), 3.96-3.78 (m, 4H), 3.76-2.75 (m, 13H), 2.73-2.54 (m, 3H), 2.47-2.36 (m, 2H), 1.62 (s, 6H), 1.38 (s, 3H), 1.26-1.04 (m, 6H). MS (ESI) m/z 650.56 [M+H]⁺

Example 221: 4-(4-fluorobenzyl)-2-(2-hydroxycyclobutyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 273A/273B)

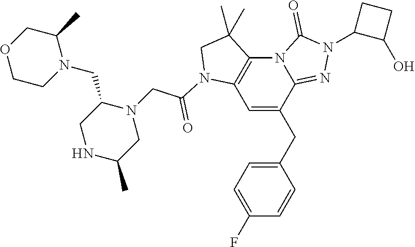 [SMILES: C[C@@H]1CN(CC(=O)N2CC(C)(C)c3c2cc(Cc2ccc(F)cc2)c2nn(C4CCC4O)c(=O)n32)[C@@H](CN2CCOC[C@H]2C)CN1]

Following the synthesis of compound 272, diastereoisomers of compound 273A and 273B was obtained separately as a white solid.
273A: 2 mg, MS (ESI) m/z 636.3 [M+H]⁺
273B: 6 mg, MS (ESI) m/z 636.3 [M+H]⁺¹H NMR (400 MHz, DMSO-d⁶) δ (ppm) 8.02 (d, J=4.4 Hz, 1H), 7.50-7.39 (m, 2H), 7.19-7.10 (m, 2H), 5.04 (dd, J=12.2, 5.9 Hz, 1H), 4.89-4.77 (m, 1H), 4.52-4.40 (m, 1H), 4.09-3.96 (m, 3H), 3.84-3.59 (m, 3H), 3.58-3.47 (m, 2H), 3.27-3.16 (m, 1H), 3.08-2.55 (m, 9H), 2.38-1.90 (m, 7H), 1.82-1.73 (m, 1H), 1.64-1.57 (m, 6H), 0.94-0.90 (m, 6H).

Example 222: 1-(4-(4-fluorobenzyl)-8,8-dimethyl-7,8-dihydro-6H-imidazo[1,5-a]pyrrolo[2,3-e]pyridin-6-yl)-2-((2R,5R)-5-methyl-2-(((R)-3-methylmorpholino)methyl)piperazin-1-yl)ethan-1-one (compound 274)

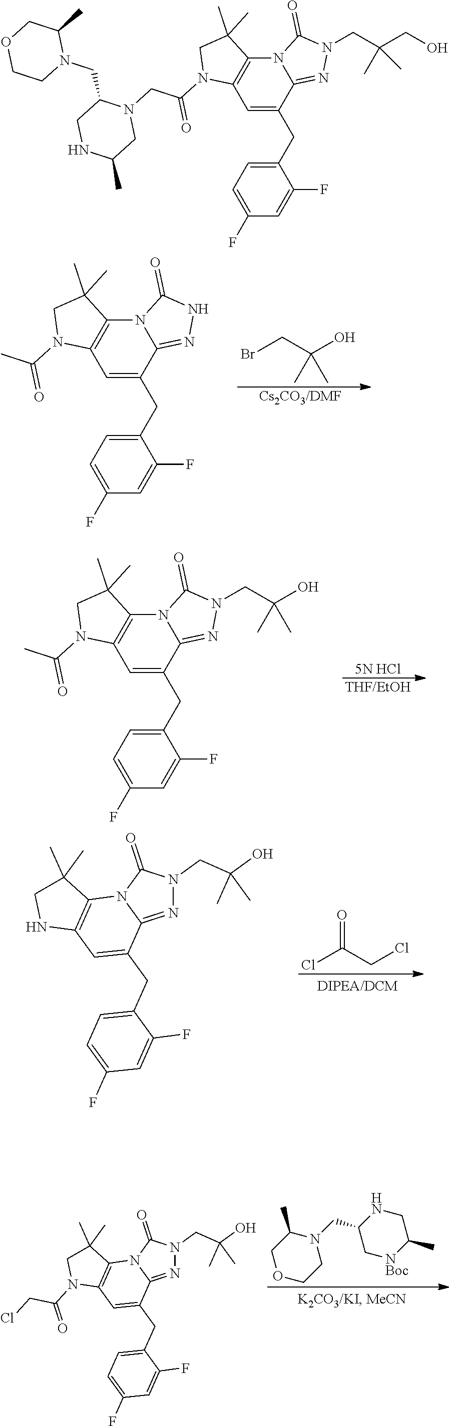

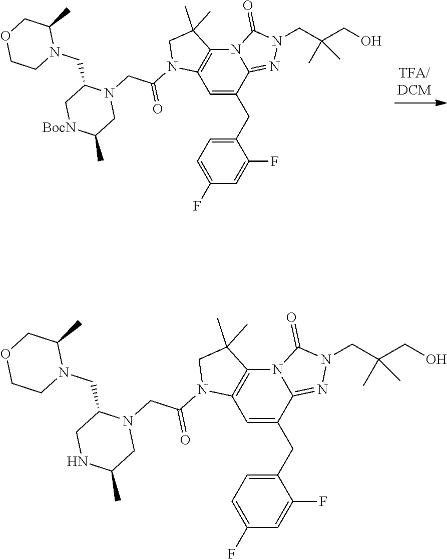 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1F)c1nn(CC(C)(C)CO)c(=O)n21 ; C[C@@H]1CN(CC(=O)N2CC(C)(C)c3c2cc(Cc2ccc(F)cc2F)c2nn(CC(C)(C)CO)c(=O)n32)[C@@H](CN2CCOC[C@H]2C)CN1]

Step 1: 6-acetyl-4-(2,4-difluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one

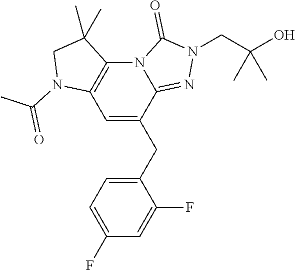 [SMILES: CC(=O)N1CC(C)(C)c2c1cc(Cc1ccc(F)cc1F)c1nn(CC(C)(C)O)c(=O)n21]

To a stirring mixture of 6-acetyl-4-(2,4-difluorobenzyl)-8,8-dimethyl-2,6,7,8-tetrahydro-1H-pyrrolo[2,3-e][1,2,4]triazolo[4,3-a]pyridin-1-one (8 g, 21.48 mmol, 1.0 eq), TBAI (0.794 g, 2.148 mmol, 0.1 eq) and Cs₂CO₃ (21.00 g, 64.5 mmol, 3.0 eq) in DMF (60 mL) was added 1-bromo-2-methylpropan-2-ol (16.44 g, 107 mmol, 5.0 eq) at room temperature. The resulting reaction mixture was stirred at 85-90° C. for 48 h. The mixture was filtered and washed with ethyl acetate. The filtrate was concentrated and the residue was diluted with water, extracted with ethyl acetate (twice). The organic layer was washed with brine, dried over Na₂SO₄ and concentrated to give a yellow oil, which was purified by silica gel flash column chromatography eluted with 60% EA/PE to give the title compound (6 g, 62.8%) as a yellow solid. MS (ESI) m/z 445.63 [M+H]⁺

Step 2: 4-(2,4-difluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one

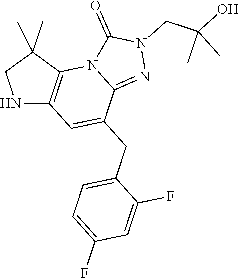 [SMILES: CC(C)(O)Cn1nc2c(Cc3ccc(F)cc3F)cc3c(n2c1=O)C(C)(C)CN3]

A mixture of above intermediate (6 g, 13.50 mmol, 1.0 eq) in 5N HCl (20 mL)/THF (20.00 mL)/ethanol (5 mL) was stirred at 80° C. for 3 h. The mixture was concentrated to give the title compound (crude, HCl salt) as a yellow solid, which was used into next step directly. MS (ESI) m/z 403.77 [M+H]⁺

Step 3: 6-(2-chloroacetyl)-4-(2,4-difluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one

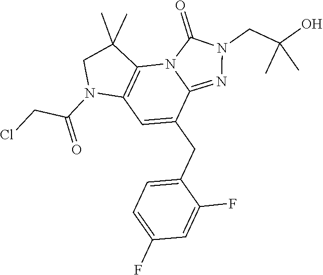 [SMILES: CC(C)(O)Cn1nc2c(Cc3ccc(F)cc3F)cc3c(n2c1=O)C(C)(C)CN3C(=O)CCl]

To a mixture of above intermediate (5.4 g, 13.42 mmol, 1.0 eq, theoretical) and N-ethyl-N-isopropylpropan-2-amine (8.67 g, 67.1 mmol, 5.0 eq) in DCM (50 mL) was added 2-chloroacetyl chloride (2.273 g, 20.13 mmol, 1.5 eq) at 0-10° C. The reaction mixture was stirred at 0-10° C. for 1 h. The mixture was quenched with water, extracted with DCM. The organic layer was washed with saturated brine, dried over Na₂SO₄ and concentrated to give a yellow oil, which was purified by silica gel flash column chromatography eluted with 60% EA/PE to give the title compound (5 g, 78% for 2 steps) as a yellow solid. MS (ESI) m/z 479.56 [M+H]⁺

Step 4: tert-butyl (2R,5S)-4-(2-(4-(2,4-difluorobenzyl)-2-(2-hydroxy-2-methylpropyl)-8,8-dimethyl-1-oxo-1,2,7,8-tetrahydro-6H-pyrrolo[2,3-e][1,2,4]triazolo[4,3-a]pyridin-6-yl)-2-oxoethyl)-2-methyl-5-(((R)-3-methylmorpholino)methyl)piperazine-1-carboxylate

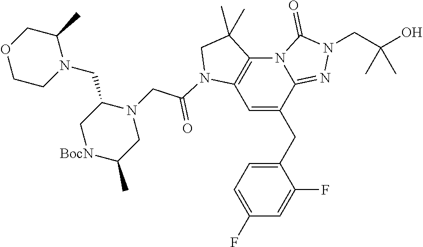 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1F)c1nn(CC(C)(C)O)c(=O)n21]

A mixture of above intermediate (5 g, 10.44 mmol, 1.0 eq), tert-butyl (2R,5S)-2-methyl-5-(((R)-3-methylmorpholino)methyl)piperazine-1-carboxylate II (4.25 g, 13.57 mmol, 1.3 eq), potassium carbonate (2.89 g, 20.88 mmol, 2.0 eq) and potassium iodide (2.60 g, 15.66 mmol, 1.5 eq) in acetonitrile (30 mL) was stirred at 25-30′C for 16 h. The mixture was diluted with water, extracted with ethyl acetate (twice). The organic layer was washed with saturated brine, dried over Na₂SO₄ and concentrated to give a yellow oil, which was purified by silica gel flash column chromatography eluted with 80% EA/PE to give the title compound (7 g, 89%) as a yellow solid. MS (ESI) m/z 756.89 [M+H]⁺

Step 5: 4-(2,4-difluorobenzyl)-2-(2-hydroxy-2-methylpropyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one

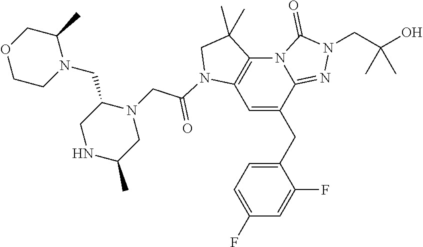 [SMILES: C[C@@H]1CN(CC(=O)N2CC(C)(C)c3c2cc(Cc2ccc(F)cc2F)c2nn(CC(C)(C)O)c(=O)n32)[C@@H](CN2CCOC[C@H]2C)CN1]

A mixture of above intermediate (7 g, 9.26 mmol, 1.0 eq) in TFA (10 mL)/DCM (15 mL) was stirred at 15-20° C. for 1 h. The mixture was concentrated. The residue was dissolved in DCM, washed with NaHCO₃(aq), dried over Na₂SO₄ and concentrated to give a yellow oil, which was purified by silica gel flash column chromatography eluted with 5% MeOH/DCM to give the target molecule (5.2 g, 86%) as a yellow solid. ¹H NMR (400 MHz, DMSO-d⁶) δ (ppm) 7.96 (s, 1H), 7.48 (dd, J=15.4, 8.6 Hz, 1H), 7.28 (td, J=10.0, 2.5 Hz, 1H), 7.08 (td, J=8.5, 2.4 Hz, 1H), 4.69 (s, 1H), 4.05-3.94 (m, 3H), 3.89-3.77 (m, 3H), 3.75-3.63 (m, 2H), 3.57-3.48 (m, 2H), 3.25-3.16 (m, 1H), 3.14-3.05 (m, 1H), 2.98-2.75 (m, 4H), 2.75-2.54 (m, 4H), 2.41-2.29 (m, 1H), 2.25-2.13 (m, 1H), 2.01-1.89 (m, 1H), 1.85-1.73 (m, 1H), 1.60 (d, J=13.4 Hz, 6H), 1.18 (s, 6H), 0.99-0.87 (m, 6H). MS (ESI) m/z 656.61 [M+H]⁺

Example 223: 4-(2,4-difluorobenzyl)-2-(1-hydroxy-2-methylpropan-2-yl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 275)

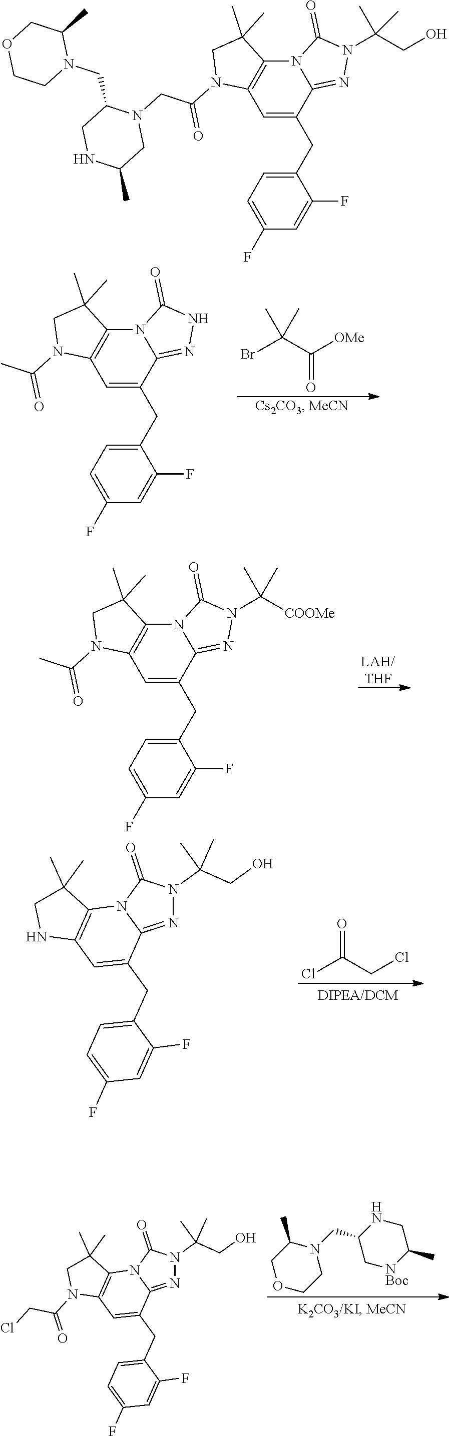

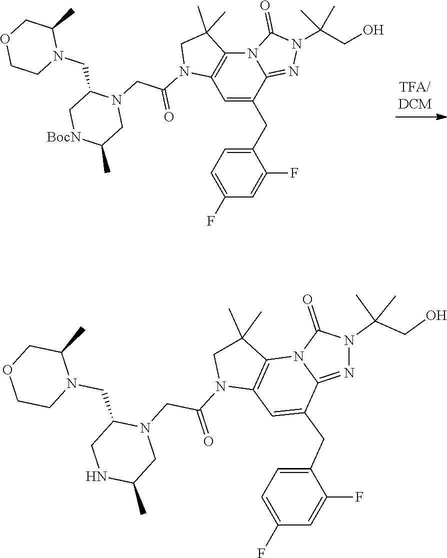 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1F)c1nn(C(C)(C)CO)c(=O)n21 ; C[C@@H]1CN(CC(=O)N2CC(C)(C)c3c2cc(Cc2ccc(F)cc2F)c2nn(C(C)(C)CO)c(=O)n32)[C@@H](CN2CCOC[C@H]2C)CN1]

Step 1: methyl 2-(6-acetyl-4-(2,4-difluorobenzyl)-8,8-dimethyl-1-oxo-7,8-dihydro-1H-pyrrolo[2,3-e][1,2,4]triazolo[4,3-a]pyridin-2(6H)-yl)-2-methylpropanoate

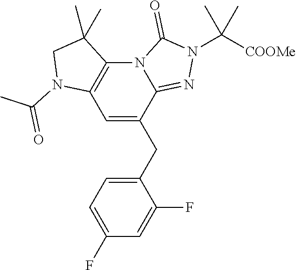 [SMILES: COC(=O)C(C)(C)n1nc2c(Cc3ccc(F)cc3F)cc3c(n2c1=O)C(C)(C)CN3C(C)=O]

A mixture of 6-acetyl-4-(2,4-difluorobenzyl)-8,8-dimethyl-2,6,7,8-tetrahydro-1H-pyrrolo[2,3-e][1,2,4]triazolo[4,3-a]pyridin-1-one (2.5 g, 6.71 mmol, 1.0 eq), methyl 2-bromo-2-methylpropanoate (3.65 g, 20.14 mmol, 3.0 eq) and cesium carbonate (5.47 g, 16.78 mmol, 2.5 eq) in acetonitrile (50 mL) was stirred at 60° C. for 6 h. The mixture was diluted with water, extracted with ethyl acetate (twice). The organic layer was washed with saturated brine, dried over Na₂SO₄ and concentrated to give a yellow oil, which was purified by silica gel flash column chromatography eluted with 60% EA/PE to give the title compound (2 g, 63.0% yield) as a yellow solid. MS (ESI) m/z 473.83 [M+H]⁺

Step 2: 4-(2,4-difluorobenzyl)-2-(1-hydroxy-2-methylpropan-2-yl)-8,8-dimethyl-2,6,7,8-tetra hydro-1H-pyrrolo[2,3-e][1,2,4]triazolo[4,3-a]pyridin-1-one

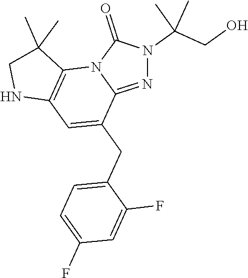 [SMILES: CC1(C)CNc2cc(Cc3ccc(F)cc3F)c3nn(C(C)(C)CO)c(=O)n3c21]

To a solution of above intermediate (2 g, 4.23 mmol, 1.0 eq) in THF (40 mL) was added LAH (0.321 g, 8.47 mmol, 2.0 eq) at 0-10° C. The reaction mixture was stirred at 0-10° C. for 1 h. The mixture was quenched with Na₂SO₄·10H₂O, stirred for 0.5 h. the mixture was filtered. The filtrate was concentrated to give the title compound (1.7 g, 100%) as a yellow oil.

MS (ESI) m/z 403.66 [M+H]⁺

Step 3: 6-(2-chloroacetyl)-4-(2,4-difluorobenzyl)-2-(1-hydroxy-2-methylpropan-2-yl)-8,8-di methyl-2,6,7,8-tetrahydro-1H-pyrrolo[2,3-e][1,2,4]triazolo[4,3-a]pyridin-1-one

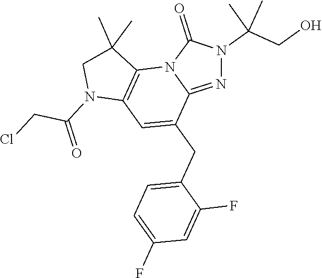 [SMILES: CC1(C)CN(C(=O)CCl)c2cc(Cc3ccc(F)cc3F)c3nn(C(C)(C)CO)c(=O)n3c21]

To a mixture of above intermediate (1.7 g, 4.22 mmol, 1.0 eq) and N-ethyl-N-isopropylpropan-2-amine (1.092 g, 8.45 mmol, 2.0 eq) in DCM (50 mL) was added 2-chloroacetyl chloride (0.716 g, 6.34 mmol, 1.5 eq) at 0-10 T. The reaction mixture was stirred at 0-10° C. for 1 h. The mixture was quenched with H₂O, extracted with DCM. The organic layer was washed with saturated brine, dried over Na₂SO₄ and concentrated to give a yellow oil, which was purified by silica gel flash column chromatography to give the title compound (1.5 g, 74.1%) as a yellow solid. MS (ESI) m/z 479.33 [M+H]⁺

Step 4: tert-butyl (2R,5S)-4-(2-(4-(2,4-difluorobenzyl)-2-(1-hydroxy-2-methylpropan-2-yl)-8,8-dimeth yl-1-oxo-1,2,7,8-tetrahydro-6H-pyrrolo[2,3-e][1,2,4]triazolo[4,3-a]pyridin-6-yl)-2-oxoethyl)-2-methyl-5-(((R)-3-methylmorpholino)methyl)piperazine-1-carboxylate

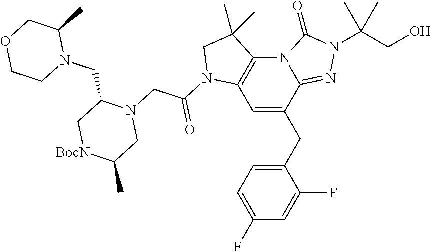 [SMILES: C[C@@H]1COCCN1C[C@H]1CN(C(=O)OC(C)(C)C)[C@H](C)CN1CC(=O)N1CC(C)(C)c2c1cc(Cc1ccc(F)cc1F)c1nn(C(C)(C)CO)c(=O)n21]

A mixture of above intermediate (1.5 g, 3.13 mmol, 1.0 eq), tert-butyl (2R,5S)-2-methyl-5-(((R)-3-methylmorpholino)methyl)piperazine-1-carboxylate II (1.276 g, 4.07 mmol, 1.3 eq), potassium carbonate (0.866 g, 6.26 mmol, 2.0 eq) and potassium iodide (0.780 g, 4.70 mmol, 1.5 eq) in acetonitrile (30 mL) was stirred at 15-20′C for 16 h. The mixture was diluted with water, extracted with ethyl acetate (twice). The organic layer was washed with saturated brine, dried over Na₂SO₄ and concentrated to give a yellow oil, which was purified by silica gel flash column chromatography eluted with 3% MeOH/DCM to give the title compound (1.4 g, 59.1%) as a yellow solid. MS (ESI) m/z 756.58 [M+H]⁺

Step 5: 4-(2,4-difluorobenzyl)-2-(1-hydroxy-2-methylpropan-2-yl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one

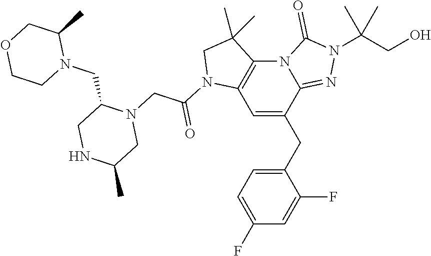 [SMILES: C[C@@H]1CN(CC(=O)N2CC(C)(C)c3c2cc(Cc2ccc(F)cc2F)c2nn(C(C)(C)CO)c(=O)n32)[C@@H](CN2CCOC[C@H]2C)CN1]

A mixture of above intermediate (1.4 g, 1.852 mmol, 1.0 eq) in TFA (10 mL)/DCM (15 mL) was stirred at 15-20° C. for 2 h. After concentration, the residue was dissolved in DCM, washed with NaHCO₃(aq), dried over Na₂SO₄ and concentrated to give a yellow oil, which was purified by silica gel flash column chromatography eluted with 5% MeOH/DCM to give the title compound (860 mg, 70.8%) as a yellow solid. ¹H NMR (400 MHz, DMSO-d⁶) δ (ppm) 7.95 (s, 1H), 7.51 (dd, J=15.4, 8.6 Hz, 1H), 7.28 (td, J=10.1, 2.5 Hz, 1H), 7.10 (td, J=8.6, 2.3 Hz, 1H), 4.83 (t, J=6.2 Hz, 1H), 4.04-3.93 (m, 3H), 3.82-3.72 (m, 3H), 3.71-3.64 (m, 2H), 3.56-3.47 (m, 2H), 3.24-3.13 (m, 1H), 3.09-2.99 (m, 1H), 2.98-2.54 (m, 8H), 2.33-2.25 (m, 1H), 2.22-2.11 (m, 1H), 1.98-1.88 (m, 1H), 1.80-1.72 (m, 1H), 1.59 (d, J=13.5 Hz, 6H), 1.54 (s, 6H), 0.95-0.88 (m, 6H). MS (ESI) m/z 656.53 [M+H]⁺

Example 224: 6-(2-((2R,5R)-2-((3-oxa-8-azabicyclo[3.2.1]octan-8-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one (compound 276)

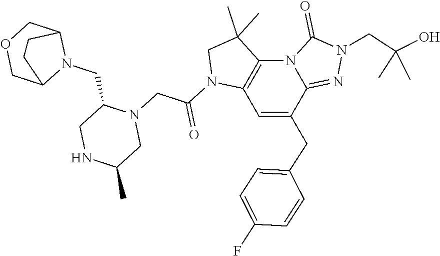 [SMILES: C[C@@H]1CN(CC(=O)N2CC(C)(C)c3c2cc(Cc2ccc(F)cc2)c2nn(CC(C)(C)O)c(=O)n32)[C@@H](CN2C3CCC2COC3)CN1]

Following the synthesis of compound 274, compound 276 was obtained as white solid (65 mg). MS (ESI) m/z 650.7 [M+H]⁺.

Example 225: 6-(2-((2R,5R)-2-((3,3-dimethylmorpholino)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one (compound 277)

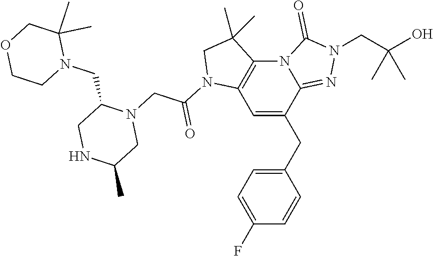 [SMILES: C[C@@H]1CN(CC(=O)N2CC(C)(C)c3c2cc(Cc2ccc(F)cc2)c2nn(CC(C)(C)O)c(=O)n32)[C@@H](CN2CCOCC2(C)C)CN1]

Following the synthesis of compound 274, compound 277 was obtained as white solid MS (ESI) m/z 652.8 [M+H]⁺.

Example 226: 6-(2-((2R,5R)-2-((7-oxa-4-azaspiro[2.5]octan-4-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one (compound 278)

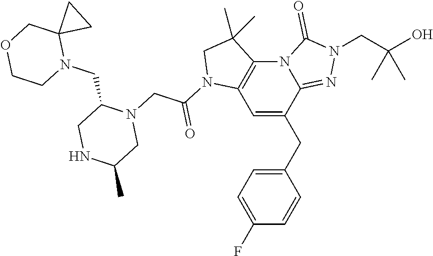 [SMILES: C[C@@H]1CN(CC(=O)N2CC(C)(C)c3c2cc(Cc2ccc(F)cc2)c2nn(CC(C)(C)O)c(=O)n32)[C@@H](CN2CCOCC23CC3)CN1]

Following the synthesis of compound 274, compound 278 was obtained as white solid. MS (ESI) m/z 650.8 [M+H]⁺.

Example 227: 3-(4-(4-fluorobenzyl)-8,8-dimethyl-6-(2-((2R,5R)-5-methyl-2-(((R)-3-methylmorpholino)methyl)piperazin-1-yl)acetyl)-7,8-dihydro-6H-pyrrolo[2,3-e][1,2,4]triazolo[1,5-a]pyridin-2-yl)oxazolidin-2-one (compound 279)

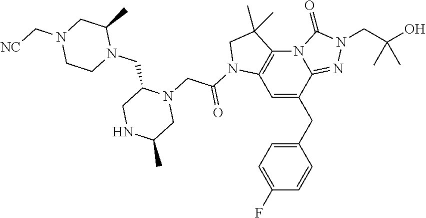 [SMILES: C[C@@H]1CN(CC(=O)N2CC(C)(C)c3c2cc(Cc2ccc(F)cc2)c2nn(CC(C)(C)O)c(=O)n32)[C@@H](CN2CCN(CC#N)C[C@H]2C)CN1]

Following the synthesis of compound 274, compound 279 was obtained as a white solid after lyophilization. MS (ESI) m/z 677.0 [M+H]⁺. ¹H NMR (400 MHz, DMSO-d⁶) δ (ppm) 8.05 (s, 1H), 7.43-7.35 (m, 2H), 7.21-7.12 (m, 2H), 4.67 (s, 1H), 4.03 (d, J=15.1 Hz, 1H), 3.95 (d, J=10.5 Hz, 2H), 3.86 (d, J=13.7 Hz, 1H), 3.82-3.73 (m, 2H), 3.70 (s, 1H), 3.68-3.55 (m, 2H), 3.48 (d, J=17.2 Hz, 1H), 3.16-3.06 (m, 1H), 3.00-2.75 (m, 2H), 2.66 (d, J=11.4 Hz, 1H), 2.61 (s, 3H), 2.56-2.54 (m, 2H), 2.49 (s, 1H), 2.36-2.22 (m, 2H), 2.20 (s, 1H), 2.04 (s, 1H), 1.89 (s, 2H), 1.72 (s, 1H), 1.66 (s, 3H), 1.57 (s, 3H), 1.15 (s, 3H), 1.13 (s, 3H), 1.01 (d, J=6.1 Hz, 3H), 0.93-0.88 (m, 3H).

Example 228: methyl (R)-4-(((2R,5R)-1-(2-(4-(4-fluorobenzyl)-2-(2-hydroxy-2-methylpropyl)-8,8-dimethyl-1-oxo-1,2,7,8-tetrahydro-6H-pyrrolo[2,3-e][1,2,4]triazolo[4,3-a]pyridin-6-yl)-2-oxoethyl)-5-methylpiperazin-2-yl)methyl)-3-methylpiperazine-1-carboxylate (compound 280)

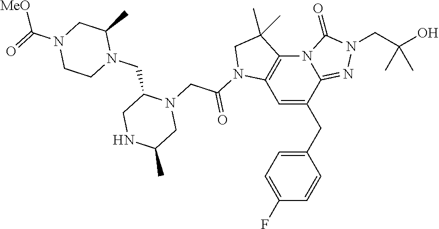 [SMILES: COC(=O)N1CCN(C[C@H]2CN[C@H](C)CN2CC(=O)N2CC(C)(C)c3c2cc(Cc2ccc(F)cc2)c2nn(CC(C)(C)O)c(=O)n32)[C@H](C)C1]

Following the synthesis of compound 274, compound 280 was obtained as a white solid (120 mg). MS (ESI) m/z 695.6 [M+H]⁺.

Example 229: 6-(2-((2R,5R)-2-(((R)-4,4-difluoro-2-methylpiperidin-1-yl) methyl)-5-methylpiperazin-1-yl)acetyl)-4-(4-fluorobenzyl)-2-(2-hydroxy-2-methyl propyl)-8,8-dimethyl-2,6,7,8-tetrahydro-1H-pyrrolo[2,3-e][1,2,4]triazolo[4,3-a]pyridin-1-one (compound 281)

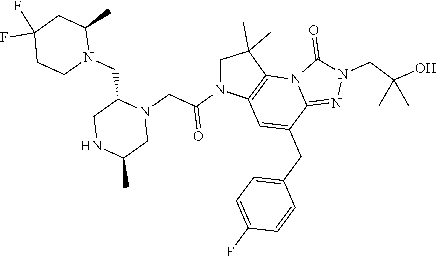 [SMILES: C[C@@H]1CN(CC(=O)N2CC(C)(C)c3c2cc(Cc2ccc(F)cc2)c2nn(CC(C)(C)O)c(=O)n32)[C@@H](CN2CCC(F)(F)C[C@H]2C)CN1]

Following the synthesis of compound 274, compound 281 was obtained as a white solid (81.8 mg). ¹H NMR (400 MHz, DMSO-d⁶) δ (ppm) 8.02 (s, 1H), 7.35 (dd, J=8.5, 5.6 Hz, 2H), 7.10 (t, J=8.9 Hz, 2H), 4.67 (s, 1H), 4.06-3.99 (m, 1H), 3.96 (s, 2H), 3.84-3.72 (m, 4H), 3.46 (d, J=16.3 Hz, 1H), 2.98 (d, J=11.3 Hz, 1H), 2.90-2.73 (m, 3H), 2.60 (d, J=8.8 Hz, 2H), 2.38-2.22 (m, 3H), 2.09-1.90 (m, 3H), 1.82 (d, J=10.1 Hz, 2H), 1.73-1.62 (m, 1H), 1.55 (s, 6H), 1.14 (s, 6H), 1.05 (d, J=6.2 Hz, 3H), 0.87 (d, J=6.1 Hz, 3H). MS (ESI) m/z 672.6 [M+H]⁺.

Example 230: 6-(2-((2R,5R)-2-(((R)-4-acetyl-2-methylpiperazin-1-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one (compound 282)

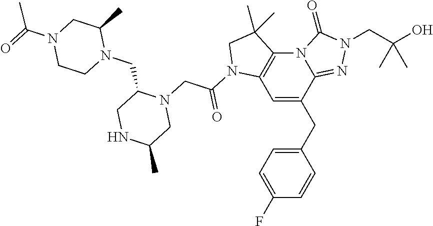 [SMILES: CC(=O)N1CCN(C[C@H]2CN[C@H](C)CN2CC(=O)N2CC(C)(C)c3c2cc(Cc2ccc(F)cc2)c2nn(CC(C)(C)O)c(=O)n32)[C@H](C)C1]

Following the synthesis of compound 174, compound 282 was obtained as a white solid after lyophilization. MS (ESI) m/z 680.6 [M+H]⁺.

Example 231: 6-(2-((2R,5R)-2-(((1S,4S)-2-oxa-5-azabicyclo[2.2.1]heptan-5-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one (compound 283)

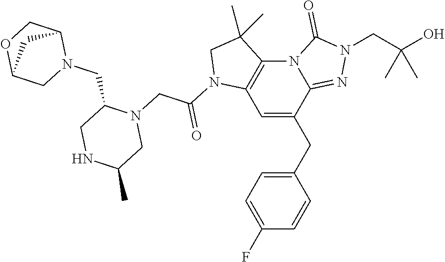 [SMILES: C[C@@H]1CN(CC(=O)N2CC(C)(C)c3c2cc(Cc2ccc(F)cc2)c2nn(CC(C)(C)O)c(=O)n32)[C@@H](CN2C[C@@H]3C[C@H]2CO3)CN1]

Following the synthesis of compound 174, compound 283 was obtained as a white solid (89 mg). ¹H NMR (400 MHz, DMSO-d⁶) δ (ppm) 8.01 (s, 1H), 7.36 (dd, J=8.6, 5.6 Hz, 2H), 7.17-7.02 (m, 2H), 4.67 (s, 1H), 4.24 (s, 1H), 4.03 (d, J=10.7 Hz, 1H), 3.93 (s, 2H), 3.86-3.78 (m, 3H), 3.78-3.67 (m, 2H), 3.41-3.39 (m, 2H), 3.31-3.30 (m, 1H), 2.84 (dd, J=11.7, 2.4 Hz, 1H), 2.71 (dd, J=13.6, 7.5 Hz, 2H), 2.61 (t, J=9.3 Hz, 2H), 2.57-2.52 (m, 1H), 2.34-2.22 (m, 4H), 1.54 (d, J=7.0 Hz, 7H), 1.43 (d, J=8.8 Hz, 1H), 1.14 (s, 6H), 0.87 (d, J=6.1 Hz, 3H). MS (ESI) m/z 636.5 [M+H]⁺.

Example 232: 6-(2-((2R,5R)-2-(((1R,4R)-2-oxa-5-azabicyclo[2.2.1]heptan-5-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one (compound 284)

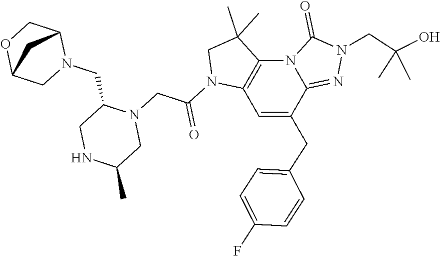 [SMILES: C[C@@H]1CN(CC(=O)N2CC(C)(C)c3c2cc(Cc2ccc(F)cc2)c2nn(CC(C)(C)O)c(=O)n32)[C@@H](CN2C[C@H]3C[C@@H]2CO3)CN1]

Following the synthesis of compound 274, compound 284 was obtained as a white solid (86.5 mg). ¹H NMR (400 MHz, DMSO-d⁶) δ (ppm) 8.01 (s, 1H), 7.35 (dd, J=8.4, 5.7 Hz, 2H), 7.11 (t, J=8.8 Hz, 2H), 4.67 (s, 1H), 4.16 (s, 1H), 4.00-3.89 (m, 3H), 3.83-3.77 (m, 3H), 3.73 (d, J=7.3 Hz, 1H), 3.62 (d, J=16.5 Hz, 1H), 3.50 (d, J=16.5 Hz, 1H), 3.41-3.38 (m, 2H), 2.79-2.69 (m, 2H), 2.69-2.54 (m, 4H), 2.44 (t, J=11.0 Hz, 1H), 2.36-2.16 (m, 3H), 1.55 (d, J=8.7 Hz, 6H), 1.41 (q, J=9.1 Hz, 2H), 1.15 (s, 6H), 0.87 (d, J=6.0 Hz, 3H). MS (ESI) m/z 636.5 [M+H]⁺.

Example 233: 2-(((S)-1,4-dioxan-2-yl)methyl)-4-(2,4-difluorobenzyl)-8,8-dimethyl-6-(2-((2R,5R)-5-methyl-2-(((R)-3-methylmorpholino)methyl)piperazin-1-yl)acetyl)-2,6,7,8-tetrahydro-1H-pyrrolo[2,3-e][1,2,4]triazolo[4,3-a]pyridin-1-one (compound 285)

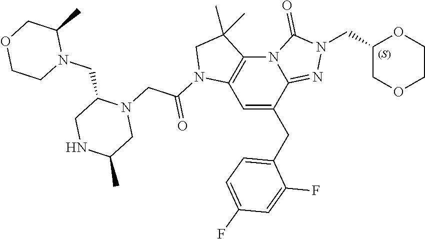

Following the synthesis of compound 274, compound 285 was obtained as a white solid (329 mg).

MS (ESI) m/z 684.5[M+H]⁺. ¹H NMR (400 MHz, DMSO-d⁶) δ (ppm) 7.95 (s, 1H), 7.52-7.41 (m, 1H), 7.32-7.22 (m, 1H), 7.14-7.01 (m, 1H), 4.06-3.90 (m, 5H), 3.87-3.81 (m, 1H), 3.80-3.72 (m, 3H), 3.69-3.56 (m, 3H), 3.54-3.45 (m, 4H), 3.41-3.36 (m, 1H), 3.23-3.14 (m, 1H), 2.98-2.88 (m, 2H), 2.87-2.74 (m, 2H), 2.69 (d, J=9.0 Hz, 1H), 2.62-2.55 (m, 2H), 2.47-2.39 (m, 1H), 2.32-2.21 (m, 1H), 2.18-2.10 (m, 1H), 1.97-1.87 (m, 1H), 1.73 (d, J=11.9 Hz, 1H), 1.57 (d, J=13.8 Hz, 6H), 0.92-0.84 (m, 6H).

Example 234: 6-(2-((2R,5R)-2-((6-oxa-3-azabicyclo[3.1.1]heptan-3-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyri din-1-one (Compound 286)

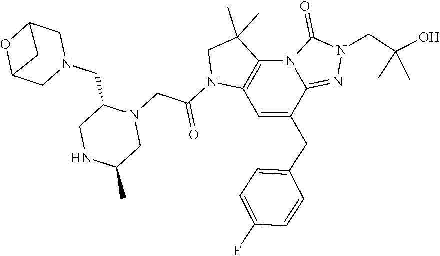 [SMILES: C[C@@H]1CN(CC(=O)N2CC(C)(C)c3c2cc(Cc2ccc(F)cc2)c2nn(CC(C)(C)O)c(=O)n32)[C@@H](CN2CC3CC(C2)O3)CN1]

Following the synthesis of compound 274, compound 286 was obtained as a white solid (91 mg) MS (ESI) m/z 636.4 [M+H]¹H NMR (400 MHz, DMSO-d⁶) δ (ppm) 7.98 (s, 1H), 7.41-7.31 (m, 2H), 7.19-7.05 (m, 2H), 4.66 (s, 1H), 4.38-4.26 (m, 2H), 4.08-3.90 (m, 3H), 3.85-3.72 (m, 4H), 3.40-3.35 (m, 1H), 3.13 (d, J=10.2 Hz, 1H), 3.01 (d, J=10.7 Hz, 1H), 2.90-2.77 (m, 2H), 2.74-2.59 (m, 4H), 2.55-2.52 (m, 1H), 2.45 (d, J=11.0 Hz, 1H), 2.36-2.19 (m, 3H), 1.93 (d, J=7.6 Hz, 1H), 1.54 (d, J=5.6 Hz, 6H), 1.15 (s, 6H), 0.88 (d, J=6.0 Hz, 3H).

Example 235: 6-(2-((2R,5R)-2-((2-oxa-5-azabicyclo[2.2.2]octan-5-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyridin-1-one (compound 287)

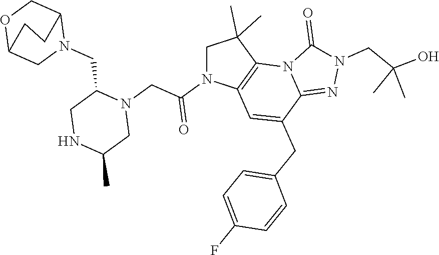 [SMILES: C[C@@H]1CN(CC(=O)N2CC(C)(C)c3c2cc(Cc2ccc(F)cc2)c2nn(CC(C)(C)O)c(=O)n32)[C@@H](CN2CC3CCC2CO3)CN1]

Following the synthesis of compound 274, compound 287 was obtained as a white solid. MS (ESI) m/z 650.6 [M+H]⁺.

Example 236: 6-(2-((2R,5R)-2-(((R)-4,4-difluoro-2-methylpyrrolidin-1-yl) methyl)-5-methylpiperazin-1-yl)acetyl)-4-(4-fluorobenzyl)-2-(2-hydroxy-2-methyl propyl)-8,8-dimethyl-2,6,7,8-tetrahydro-1H-pyrrolo[2,3-e][1,2,4]triazolo[4,3-a]pyridin-1-one (compound 288)

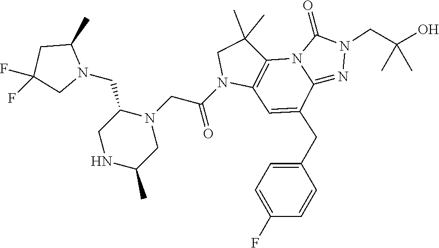 [SMILES: C[C@@H]1CN(CC(=O)N2CC(C)(C)c3c2cc(Cc2ccc(F)cc2)c2nn(CC(C)(C)O)c(=O)n32)[C@@H](CN2CC(F)(F)C[C@H]2C)CN1]

Following the synthesis of compound 274, compound 288 was obtained as a white solid. MS (ESI) m/z 659.2 [M+H]⁺. ¹H NMR (400 MHz, DMSO) δ (ppm) 8.05 (d, J=17.8 Hz, 1H), 7.42-7.33 (m, 2H), 7.14 (t, J=8.8 Hz, 2H), 4.70 (d, J=2.8 Hz, 1H), 4.06-3.94 (m, 4H), 3.89-3.75 (m, 4H), 3.66-3.48 (m, 3H), 2.90 (dd, J=10.7, 2.1 Hz, 1H), 2.87-2.69 (m, 2H), 2.68-2.54 (m, 4H), 2.46-2.30 (m, 2H), 2.28 (s, 1H), 1.93-1.66 (m, 2H), 1.60 (d, J=3.1 Hz, 3H), 1.58 (s, 3H), 1.56-1.48 (m, 1H), 1.32 (dd, J=16.9, 12.1 Hz, 1H), 1.19 (s, 3H), 1.18 (s, 3H), 0.90 (d, J=6.0 Hz, 3H).

Example 237: 6-(2-((2R,5R)-2-((3-oxa-6-azabicyclo[3.1.1]heptan-6-yl)methyl)-5-methylpiperazin-1-yl)acetyl)-4-(4-fluorobenzyl)-2-(2-hydroxy-2-methylpropyl)-8,8-dimethyl-2,6,7,8-tetrahydro-1H-pyrrolo[2,3-e][1,2,4]triazolo[4,3-a]pyri din-1-one (compound 289)

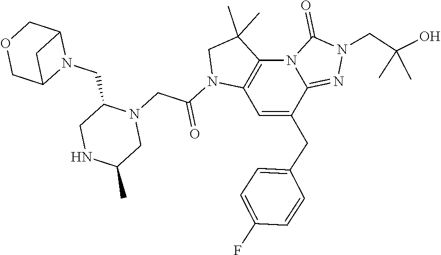 [SMILES: C[C@@H]1CN(CC(=O)N2CC(C)(C)c3c2cc(Cc2ccc(F)cc2)c2nn(CC(C)(C)O)c(=O)n32)[C@@H](CN2C3COCC2C3)CN1]

Following the synthesis of compound 274, compound 289 was obtained as a white solid (4.7 mg). MS (ESI) m/z 636.6 [M+H]⁺

BIOLOGICAL EXAMPLES

Example 238: Cell Proliferation Assay

MDA-MB-231 and EVSA-T cells (10000 cells/well) were plated onto 96-well plates (Corning, Cat #3917) in 95 μL of medium (RPMI-1640 containing 10% FBS and MEM containing 10% FBS, respectively). Compounds with various concentrations (0.0005, 0.001, 0.004, 0.01, 0.04, 0.1, 0.3, 1.1, 3.3, 10 M) were added to the wells. After treatment for 72 h, 30 μL of Celltiter-Glo reagent (Promega, Cat #G7573) was added and incubated at room temperature for 15 min, then the luminescent signal was determined using a microplate reader (Biotek, SynergyH1). The inhibitor dose-response curves were analyzed using normalized IC₅₀ regression curve fitting with control based normalization.

The data for some exemplary compounds are shown in Table 1 below.

TABLE 1

| | MDA-MB-231 | EVSA-T |
|---|---|---|
| Compound No. | IC₅₀ (μM) | IC₅₀ (μM) |
| 1 | 0.077 | 0.007 |
| 2 | 0.11 | 0.013 |
| 3 | 0.059 | — |
| 4 | 0.097 | — |
| 5 | 0.16 | — |
| 6 | 0.05 | — |
| 7 | 0.14 | — |
| 8 | 0.036 | — |
| 9 | 0.53 | 0.018 |
| 10 | 0.35 | 0.027 |
| 11 | 0.17 | 0.01 |
| 12 | 0.19 | 0.012 |
| 13 | 0.17 | 0.009 |
| 14 | 0.042 | 0.006 |
| 15 | >10 | — |
| 16 | 6.26 | — |
| 17 | 0.12 | 0.004 |
| 18 | 0.074 | 0.014 |
| 19 | 0.18 | 0.021 |
| 20 | 0.11 | 0.013 |
| 21 | 0.023 | 0.005 |
| 22 | 3.71 | — |
| 23 | 0.32 | — |
| 24 | 0.24 | — |
| 25 | 0.084 | 0.011 |
| 26 | 0.18 | — |
| 27 | 0.26 | — |
| 28 | 0.33 | — |
| 29 | 0.10 | 0.01 |
| 30 | 0.016 | 0.003 |
| 31 | 0.37 | 0.003 |
| 32 | 0.06 | 0.02 |
| 36 | 0.036 | 0.002 |
| 37 | 0.071 | 0.004 |
| 38 | 0.15 | 0.006 |
| 39 | 0.15 | 0.034 |
| 40 | 0.059 | 0.003 |
| 41 | 0.078 | 0.014 |
| 42 | 0.10 | 0.013 |
| 44 | 0.075 | 0.009 |
| 45 | 0.18 | 0.04 |
| 49 | 0.03 | 0.005 |
| 50 | >10 | — |
| 53 | 0.13 | 0.06 |
| 56 | 0.035 | 0.009 |
| 63 | 0.028 | 0.003 |
| 75 | 0.087 | — |
| 77 | 0.070 | 0.005 |
| 78 | 0.12 | — |
| 80 | 0.17 | — |
| 91 | 0.08 | — |
| 94 | 0.026 | 0.004 |
| 95 | 0.012 | 0.0004 |
| 96 | 0.052 | 0.005 |
| 97 | 0.047 | 0.003 |
| 98 | 0.072 | 0.008 |
| 99 | 0.012 | 0.005 |
| 100 | 0.021 | 0.004 |
| 101 | 0.023 | 0.008 |
| 105 | 0.10 | 0.009 |
| 106 | 0.39 | — |
| 109 | 0.061 | 0.009 |
| 111 | 0.042 | 0.002 |
| 113 | 0.074 | 0.006 |
| 115 | 0.16 | 0.03 |
| 116 | 0.093 | 0.019 |
| 117 | >10 | — |
| 118 | 0.12 | 0.01 |
| 119 | 0.10 | 0.006 |
| 120 | 0.40 | — |
| 121 | 0.065 | 0.005 |
| 122 | 0.065 | 0.006 |
| 123 | 0.12 | 0.043 |
| 124 | 0.29 | — |
| 125 | 0.20 | — |
| 126 | 0.02 | 0.003 |
| 127 | 3.08 | — |
| 128 | 0.012 | 0.002 |
| 129 | 0.046 | 0.011 |
| 130 | 0.23 | 0.037 |
| 131 | 2.74 | — |
| 132 | 0.22 | 0.056 |
| 133 | 0.21 | 0.056 |
| 134 | 0.19 | 0.083 |
| 135 | 0.040 | 0.004 |
| 136 | 1.39 | — |
| 137 | 0.19 | 0.012 |
| 138 | 0.16 | 0.033 |
| 139 | 0.71 | — |
| 140 | 0.045 | 0.003 |
| 141 | 0.31 | — |
| 142 | 0.05 | 0.002 |
| 143 | 0.043 | 0.003 |
| 144 | 0.051 | 0.003 |
| 145 | 0.45 | — |
| 146 | 0.27 | — |
| 147 | 0.12 | 0.01 |
| 148 | 0.1 | 0.006 |
| 149 | 0.1 | 0.007 |
| 150 | 0.49 | — |
| 151 | 3.8 | — |
| 152 | 0.17 | 0.03 |
| 153 | 0.038 | 0.005 |
| 154 | 0.1 | 0.005 |
| 155 | 0.72 | — |
| 156 | 1.33 | — |
| 157 | 0.2 | — |
| 158 | 0.72 | — |
| 159 | 0.02 | 0.004 |
| 160 | 0.33 | — |
| 161 | 0.12 | 0.03 |
| 162 | 0.046 | 0.01 |
| 163 | 0.028 | 0.002 |
| 164 | 0.43 | — |
| 165 | 0.021 | 0.004 |
| 166 | 0.15 | — |
| 167 | 0.12 | 0.016 |
| 168 | 0.19 | — |
| 169 | 0.98 | — |
| 170 | 0.099 | — |
| 171 | 0.095 | — |
| 172 | 0.61 | — |
| 173 | 0.53 | — |
| 174 | 0.45 | — |
| 175 | 0.12 | — |
| 176 | 0.19 | — |
| 177 | 0.32 | — |
| 178 | 0.49 | — |
| 179 | 0.18 | 0.015 |
| 180 | 0.615 | 0.056 |
| 181 | 0.097 | 0.015 |
| 182 | 0.017 | 0.002 |
| 183 | 0.81 | — |
| 184 | 0.017 | 0.006 |
| 185 | 0.023 | 0.007 |
| 186 | 0.23 | — |
| 187 | 0.008 | 0.006 |
| 188 | 0.08 | — |
| 189 | 0.01 | 0.002 |
| 190 | 0.014 | 0.003 |
| 191 | 0.08 | — |
| 192 | 0.038 | 0.007 |
| 193 | 0.05 | 0.006 |
| 194 | 0.68 | — |
| 195 | 0.18 | — |
| 196 | 0.043 | 0.004 |
| 197 | >10 | — |
| 198 | 1.0 | — |
| 199 | 0.02 | 0.006 |
| 200 | 0.07 | — |
| 201 | 0.026 | 0.011 |
| 202 | 0.034 | 0.017 |
| 203 | 0.01 | 0.003 |
| 204 | 0.044 | 0.014 |
| 205 | 7.08 | — |
| 206 | 0.018 | 0.002 |
| 207 | 0.02 | 0.002 |
| 208 | 0.21 | — |
| 209 | 2.16 | — |
| 210 | 0.033 | 0.003 |
| 211 | 0.15 | — |
| 212 | 0.041 | 0.007 |
| 213 | 0.56 | — |
| 214 | 0.049 | 0.008 |
| 215 | 0.43 | — |
| 216 | 0.28 | — |
| 217 | 0.35 | — |
| 218 | 0.047 | 0.002 |
| 219 | 0.047 | 0.002 |
| 220 | 0.068 | 0.002 |
| 221 | 0.19 | — |
| 222 | 0.11 | 0.004 |
| 223 | 0.077 | 0.003 |
| 224 | 0.12 | 0.002 |
| 225 | 0.05 | 0.002 |
| 226 | 0.53 | — |
| 227 | 0.082 | 0.002 |
| 228 | 0.009 | 0.002 |
| 229 | 0.076 | 0.007 |
| 230 | 0.038 | 0.008 |
| 231 | 0.028 | 0.004 |
| 232 | 0.33 | — |
| 233 | 0.26 | — |
| 234 | 0.010 | 0.0007 |
| 235 | 0.038 | 0.0013 |
| 236 | 0.087 | 0.0007 |
| 237 | 0.046 | 0.0015 |
| 238 | 0.031 | 0.0013 |
| 239 | 0.014 | 0.0005 |
| 240 | 0.025 | 0.0059 |
| 241 | 0.015 | 0.0063 |
| 242 | 0.018 | 0.0027 |
| 243 | 0.095 | 0.0145 |
| 244 | 0.003 | 0.0046 |
| 245 | 0.005 | 0.0062 |
| 246 | 0.004 | 0.0035 |
| 247 | 0.004 | 0.0062 |
| 248 | 0.047 | — |
| 249 | 0.041 | — |
| 250 | 0.018 | 0.0061 |
| 251 | 0.13 | 0.0296 |
| 252 | 0.032 | 0.0053 |
| 253 | 0.23 | — |
| 254 | 0.069 | 0.0038 |
| 255 | 0.58 | — |
| 256 | 0.07 | 0.0089 |
| 257 | 0.14 | 0.0116 |
| 258 | 0.195 | — |
| 259 | 0.087 | 0.0158 |
| 260 | 0.029 | 0.0027 |
| 261 | 0.017 | 0.0032 |
| 262 | 0.0125 | 0.0013 |
| 263 | 0.027 | 0.0082 |
| 264 | 0.015 | 0.0047 |
| 265 | 0.041 | 0.0268 |
| 266 | 0.016 | 0.0049 |
| 267 | 0.034 | 0.0196 |
| 268 | 0.35 | — |
| 269 | 0.010 | 0.0051 |
| 270 | 0.051 | 0.0115 |
| 271 | 0.022 | 0.0022 |
| 272 | 0.021 | 0.002 |
| 273A/B | 0.023/0.014 | 0.00124/0.0017 |
| 274 | 0.009 | — |
| 275 | 0.009 | 0.0031 |
| 276 | 0.39 | — |
| 277 | 0.067 | — |
| 278 | 0.039 | 0.0097 |
| 279 | 0.055 | — |
| 280 | 0.068 | — |
| 281 | 0.026 | — |
| 282 | 0.66 | — |
| 283 | 0.36 | — |
| 284 | 0.25 | — |
| 285 | 0.026 | 0.0046 |
| 286 | 0.27 | — |
| 287 | 0.48 | — |
| 288 | 0.029 | — |
| 289 | 1.7 | — |

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof.

All patents, patent applications, and publications cited herein are fully incorporated by reference herein in their entirety.

1. A compound of Formula I: 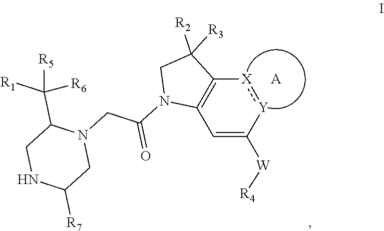
wherein:
W is O, S, S(O)₂, NR^{a}, —C(═O) or CR^{a}R^{b}, wherein R^{a} and R^{b} are each independently selected from H, halo, C₁-C₆ alkyl, OH or C₃-C₆ cycloalkyl;
X and Y are each independently C or N;
------ represents a single bond or a double bond;
R₁ is selected from heterocyclyl or heteroaryl optionally substituted with one or more groups selected from R_{1A}; and R_{1A} is selected from CN, —C₁-C₆ alkyl-CN, —C(═O)—NH₂, halo, OH, COOH, NH₂, oxo, C₁-C₆ alkyl, C₁-C₆ haloalkyl, C₁-C₆ alkoxyl, —C(═O)—C₁-C₆ alkyl, —C(═O)—O—C₁-C₆ alkyl, —C₁-C₆ alkyl-C₁-C₆ alkoxy, —C₁-C₆ alkyl-OH, —C₁-C₆ alkyl-NH₂, —S(O)₂—C₁-C₆ alkyl, or 4-6-membered heterocyclyl; or two R_{1A} together with the carbon atom to which they are attached form a C₃-C₆ cycloalkyl or —C(═O);
R₂ and R₃ are each independently selected from H, or C₁-C₆ alkyl; or R₂ and R₃ together with the carbon atom to which they are attached form a C₃-C₆ cycloalkyl or C₃-C₆ heterocyclyl;
R₄ is selected from phenyl, C₂-C₆ alkyl, 3-6-membered cycloalkyl, 3-6-membered heterocycloalkyl or 5-6-membered heteroaryl optionally substituted with one or more R₄ₐ, or C₁-C₆ alkyl or —C₁-C₆ alkyl-C₃-C₆ cycloalkyl; and R₄ₐ is selected from CN, halo, OH, NH₂, oxo, COOH, C₁-C₆ alkyl, C₁-C₆ haloalkyl, C₁-C₆ alkoxyl; optionally, R^{a} and R₄ₐ together with the atoms to which they are attached may form a 5-6-membered carbocyclyl, 5-6-membered heterocyclyl or 5-6-membered heteroaryl optionally substituted with one or more of halo, C₁-C₆ alkyl, or OH;
R₅ and R₆ are each independently selected from H, or C₁-C₆ alkyl;
R₇ is selected from C₁-C₆ alkyl, preferably C₁-C₃ alkyl;
A ring is selected from 5-8-membered aryl, 5-8-membered heteroaryl or 5-8-membered heterocyclyl optionally substituted with one or more groups selected from R₈; and R₈ is selected from C₁-C₆ alkyl, deuterated C₁-C₆ alkyl, C₁-C₆ haloalkyl, —C₁-C₆ haloalkyl-OH, C₁-C₆ alkoxy, C₁-C₆ alkyl(C₁-C₆ alkoxy)-C₁-C₆ alkoxy, C₃-C₆ cycloalkyl, —C₁-C₆ alkyl-C₃-C₆cycloalkyl, oxo, 4-7-membered heterocyclyl, —NH-4-7-membered heterocyclyl, —C₁-C₆ alkyl-4-7-membered heterocyclyl, —C₁-C₆ alkyl(OH)-4-7-membered heterocyclyl, CN, —C₁-C₆ alkyl-CN, —COOH, —OH, amino, halo, —NH—S(O)₂—C₁-C₆ alkyl, —N(S(O)₂—C₁-C₆ alkyl)₂, —C₁-C₆ alkyl-OH, —C₁-C₆ haloalkyl-OH, —C₁-C₆ alkyl (OH)—OH, —S(O)₂—C₁-C₆ alkyl, —C₁-C₆ alkyl-S(O)₂—C₁-C₆ alkyl, —P(O)—(C₁-C₆ alkyl)₂, —C₁-C₆ alkyl-N(C₁-C₆ alkyl)₂, —C(═O)—NH₂, —C₁-C₆ alkyl-C(═O)—NH₂, —C(═O)—NH—C₁-C₆ alkyl, —C(═O)—NH—C₃-C₆ cycloalkyl, —C(═O)—NH-(deuterated C₁-C₆ alkyl), —C(═O)—N(C₁-C₆ alkyl)₂, —C(═O)—O—C₁-C₆ alkyl, —C(═O)—C₁-C₆ alkyl, —NH—C(═O)—C₁-C₆ alkyl, —NH—C(═O)—H, —N(C₁-C₆ alkyl)-C(═O)—H, —NH—C(═O)—O—C₁-C₆ alkyl, —N(C₁-C₆ alkyl)-C(═O)—C₁-C₆ alkyl, 5-6-membered heteroaryl, —C₁-C₆ alkyl-(5-6-membered heteroaryl), or phenyl, wherein the C₃-C₆ cycloalkyl, 4-7-membered heterocyclyl, 5-6-membered heteroaryl or phenyl is optionally substituted with one or more R₈ₐ, and R₈ₐ is selected from halo, —OH, C₁-C₆ alkyl, or oxo;
or A ring is absent;
or a pharmaceutically acceptable salt or solvate thereof.

2. The compound of claim 1, wherein:
W is O, S, NR^{a}, or CR^{a}R^{b}, wherein R^{a} and R^{b} are each independently selected from H, halo, C₁-C₆ alkyl, OH or C₃-C₆ cycloalkyl;
X and Y are each independently C or N;
------ represents a single bond or a double bond;
R₁ is selected from heterocyclyl or heteroaryl optionally substituted with one or more groups selected from R_{1A}; and R_{1A} is selected from CN, halo, OH, COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH, —C₁-C₆ alkyl-NH₂, or two R_{1A} together with the carbon atom to which they are attached form a
C₃-C₆ cycloalkyl or —C(═O);
R₂ and R₃ are each independently selected from H, or C₁-C₆ alkyl; or R₂ and R₃ together with the carbon atom to which they are attached form a C₃-C₆ cycloalkyl;
R₄ is selected from phenyl or pyridyl optionally substituted with one or more R₄ₐ, or C₁-C₆ alkyl or —C₁-C₆ alkyl-C₃-C₆ cycloalkyl; and R₄ₐ is selected from CN, halo, OH, NH₂, COOH, C₁-C₆ alkyl, C₁-C₆ haloalkyl, C₁-C₆ alkoxyl;
R₅ and R₆ are each independently selected from H, or C₁-C₆ alkyl;
R₇ is selected from C₁-C₆ alkyl, preferably C₁-C₃ alkyl;
A ring is selected from 5-8-membered aryl or 5-8-membered heteroaryl optionally substituted with one or more groups selected from R₈; and R₈ is selected from C₁-C₆ alkyl, C₁-C₆ haloalkyl, C₁-C₆ alkoxy, C₃-C₆ cycloalkyl, 5- or 6-membered heterocyclyl containing one or more heteroatoms selected from N, O and S, CN, —COOH, —OH, amino, halo, —NH—S(O)₂—C₁-C₆ alkyl, —N(S(O)₂—C₁-C₆ alkyl)₂, —C₁-C₆ alkyl-OH, —C(═O)—NH₂, —C(═O)—NH—C₁-C₆ alkyl, —C(═O)—O—C₁-C₆ alkyl, —NH—C(═O)—C₁-C₆ alkyl, phenyl optionally substituted with one or more halogens;
or a pharmaceutically acceptable salt or solvate thereof.

3. The compound of claim 2, wherein the compound is of Formula II: 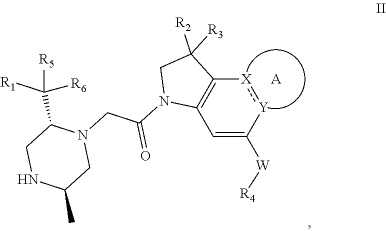
or a pharmaceutically acceptable salt or solvate thereof.

4. The compound of claim 2, wherein the compound is of Formula II-A, II-B or II-C; 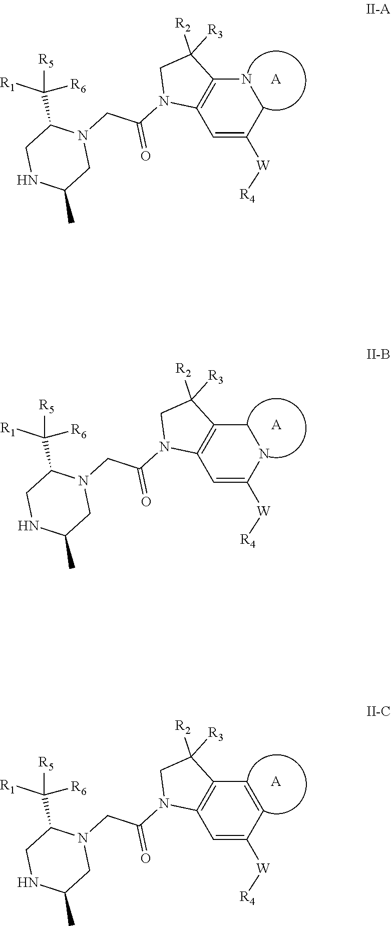
or a pharmaceutically acceptable salt or solvate thereof.

5. The compound of claim 4, wherein the A ring contains 1, 2 or 3 nitrogen atoms.

6. The compound of claim 1, wherein
(i) X is N, Y is C, and the 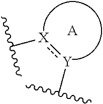
is selected from: 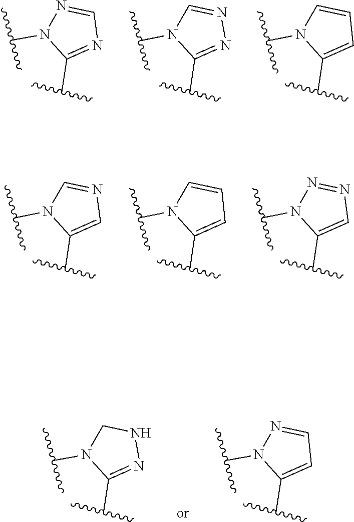
optionally substituted with one or more groups selected from R₈; or
(ii) X is C, Y is N, and the 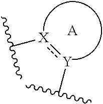
is selected from: 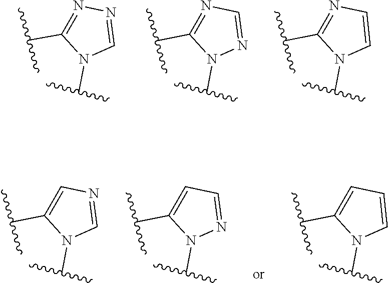
optionally substituted with one or more groups selected from R₈; or
(iii) X is C, Y is C, and the 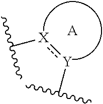
is selected from: 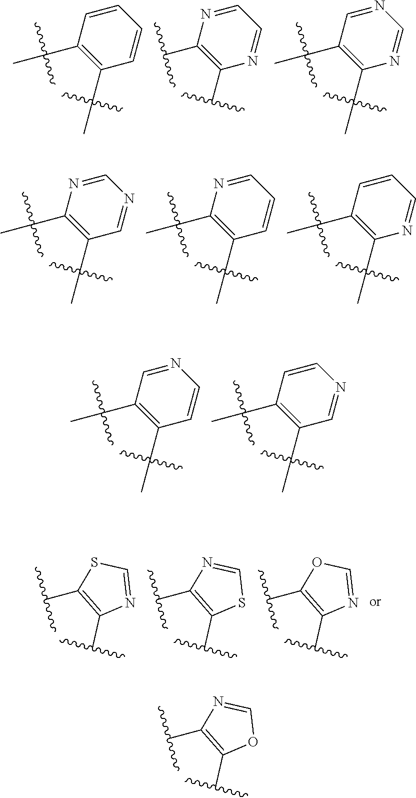
optionally substituted with one or more groups selected from R₈.

7-9. (canceled)

10. The compound of claim 4, wherein 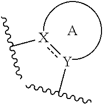
is selected from the group consisting of:

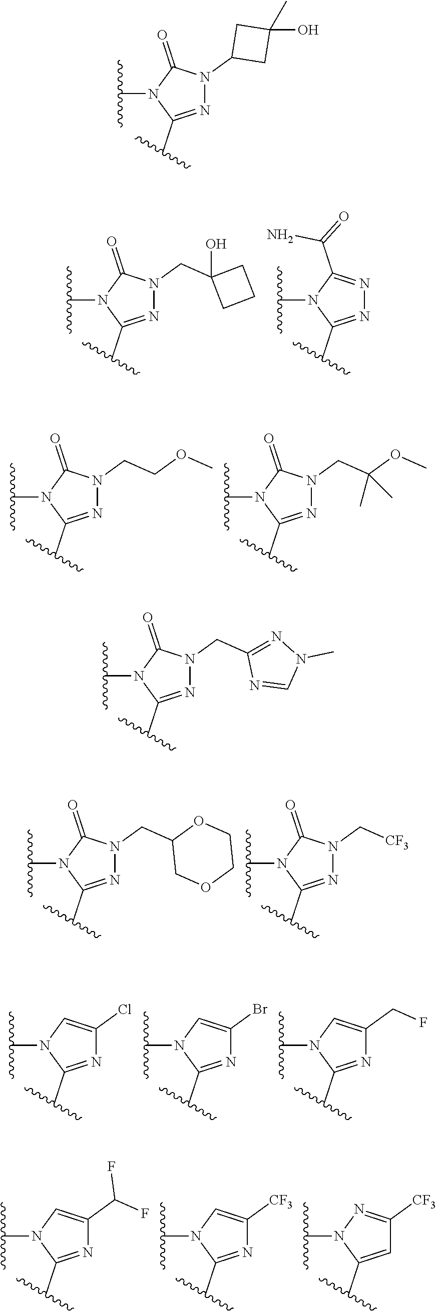

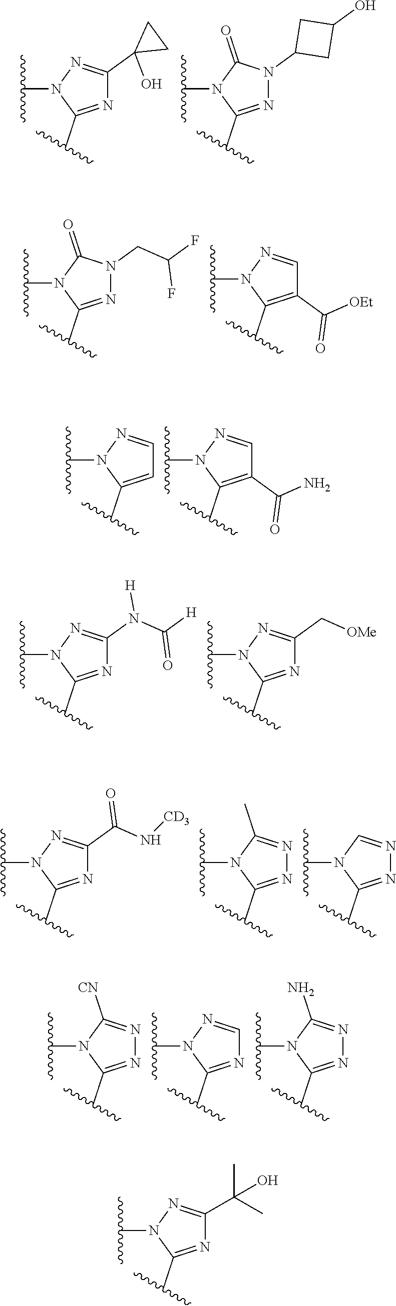

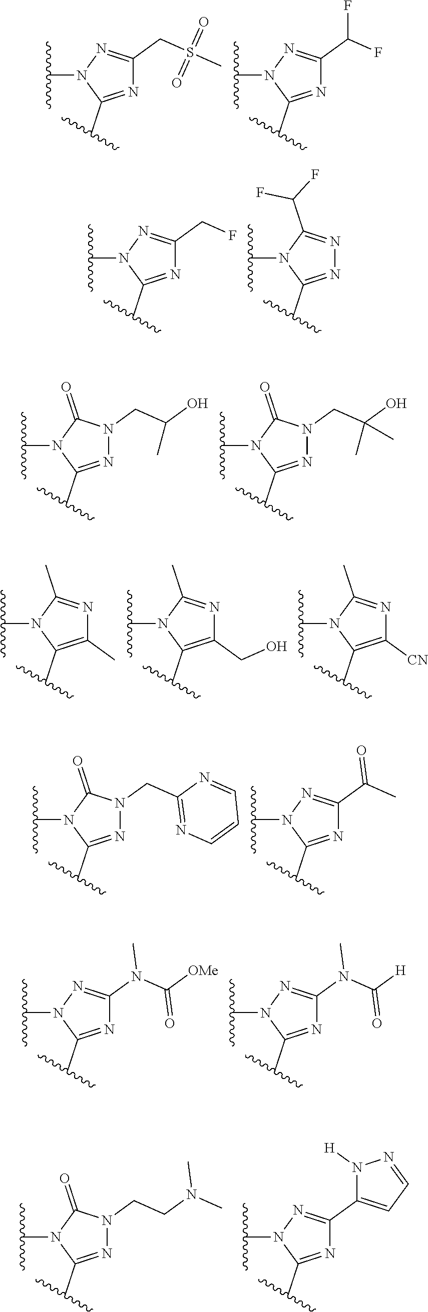

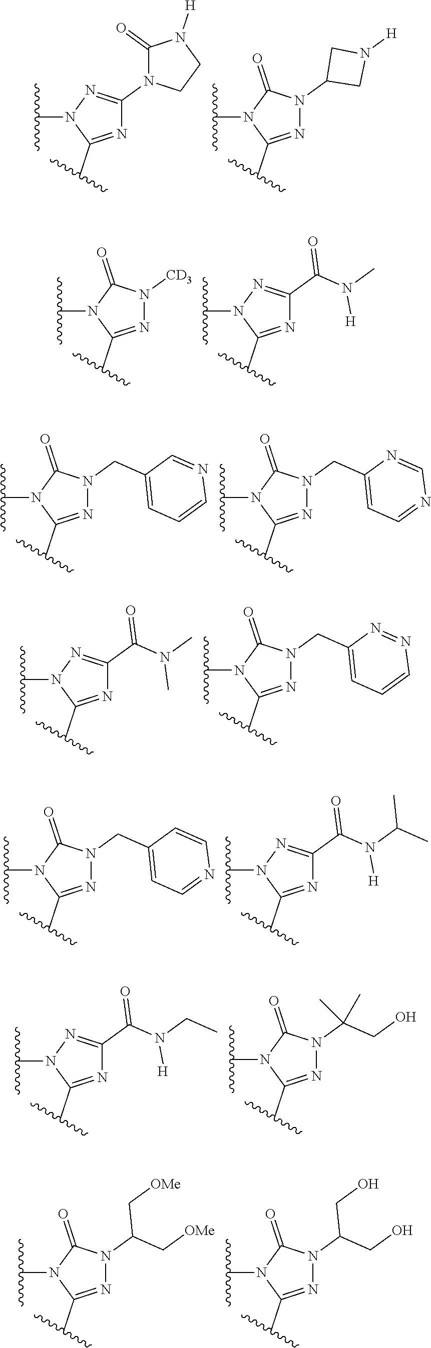

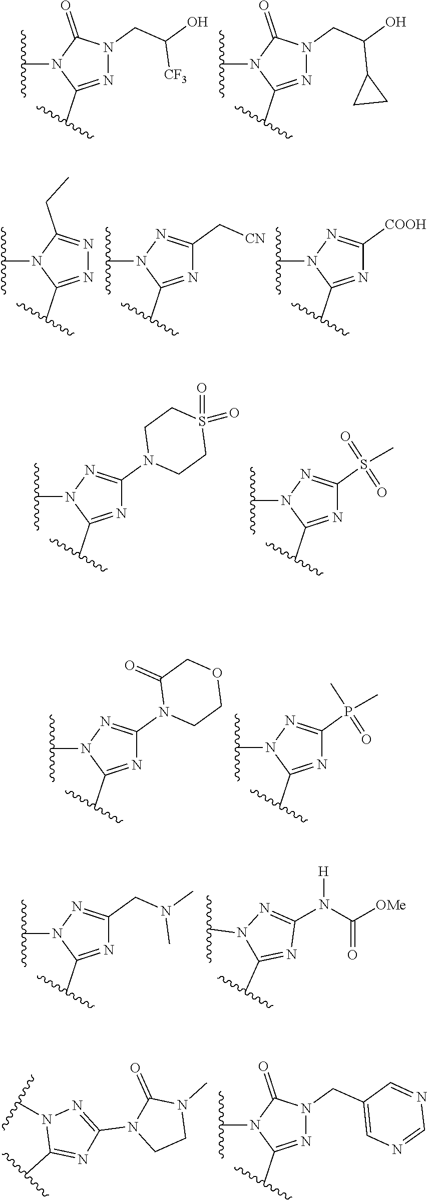

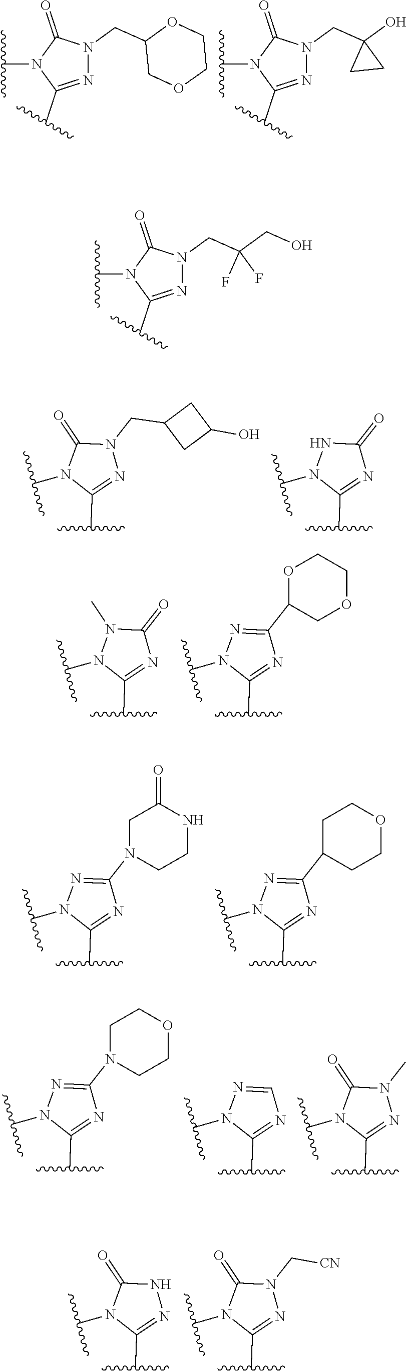

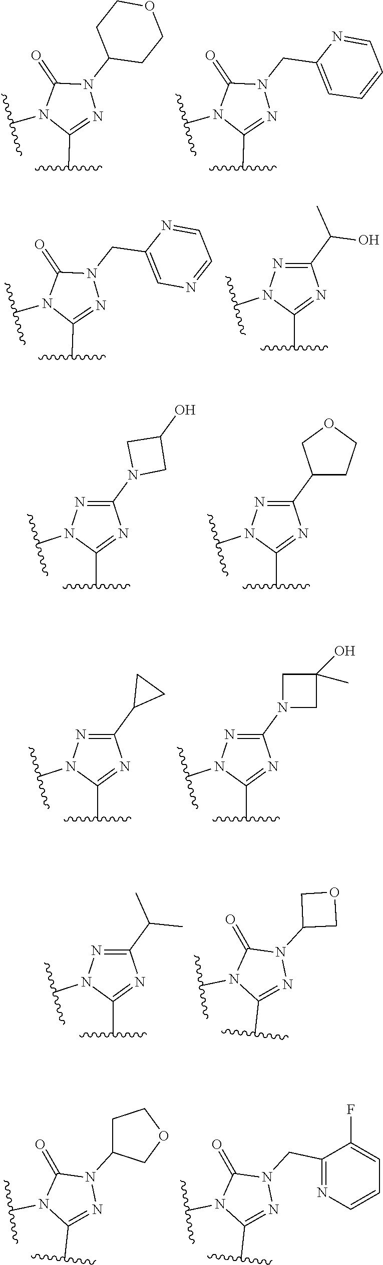

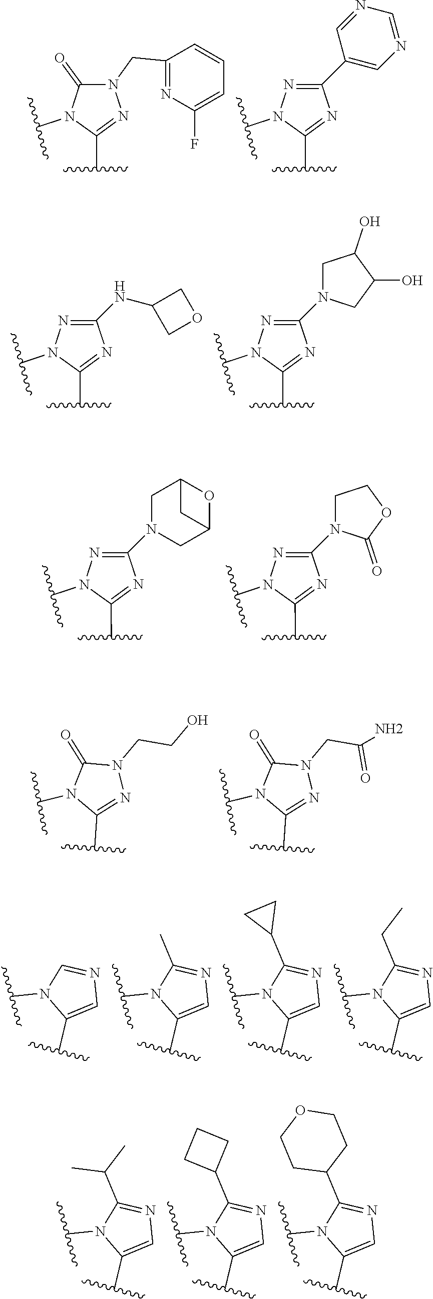

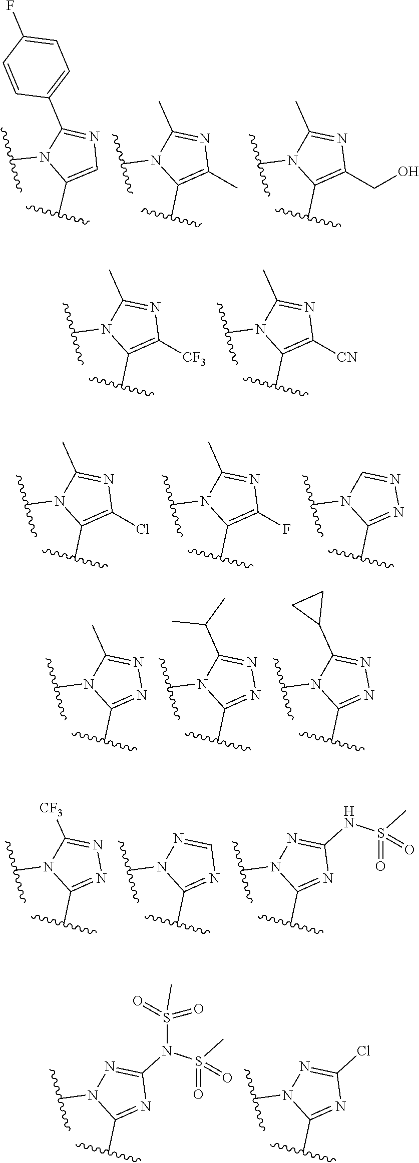

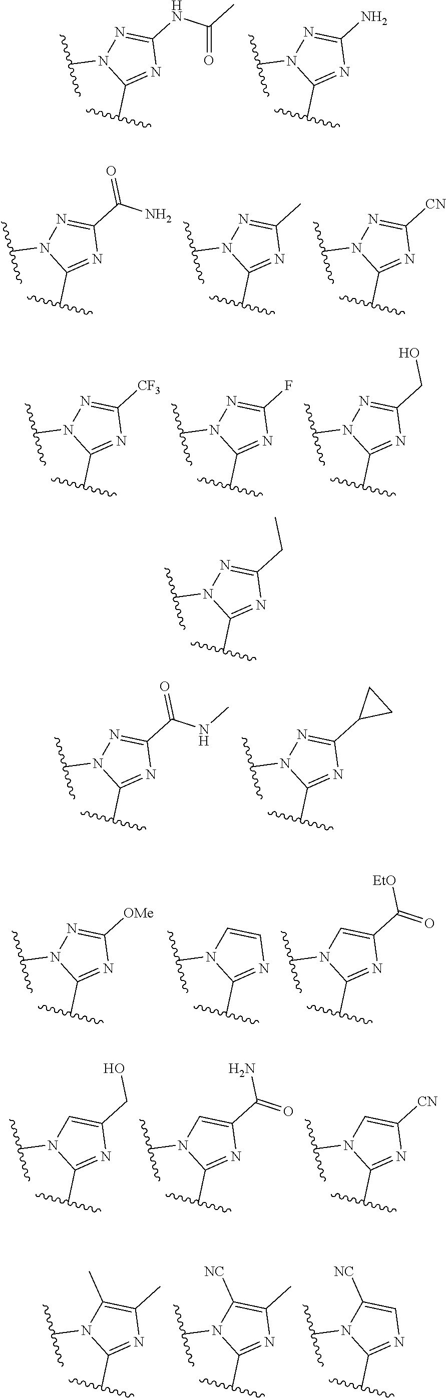

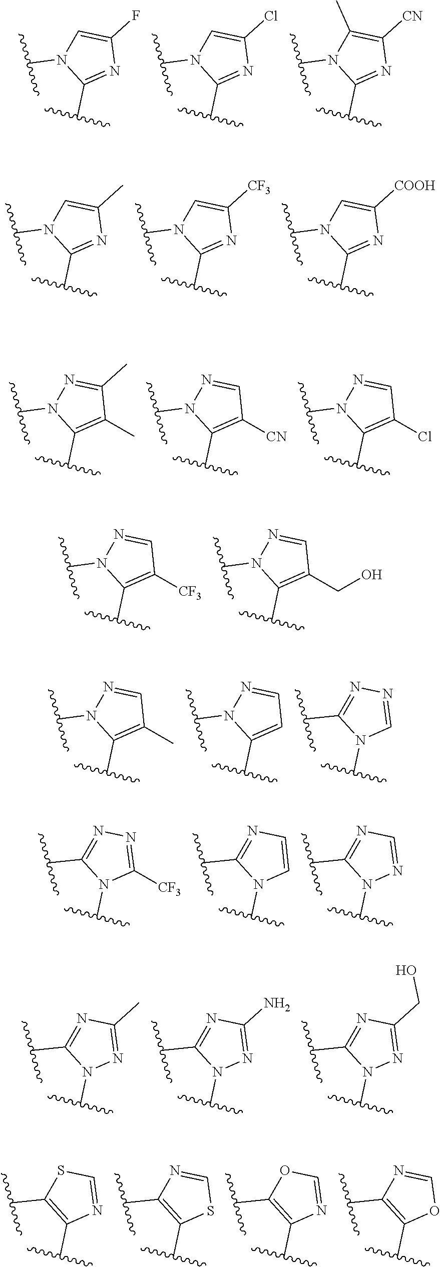

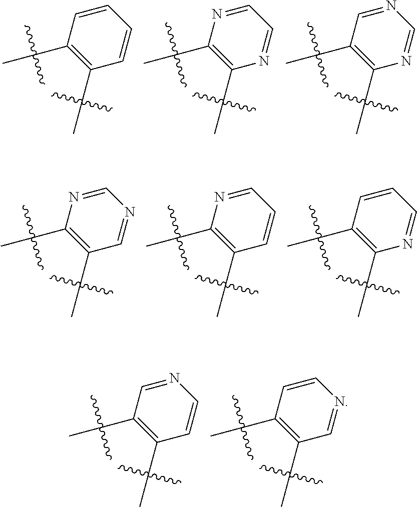

11. (canceled)

12. The compound of claim 1, wherein R₁ is of Formula III: 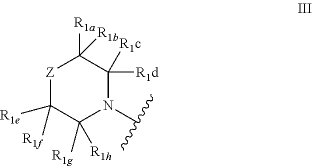
wherein:
Z is selected from O, S, NR^{a}, or CR^{a}R^{b}, wherein R^{a} and R^{b} are each independently selected from CN, halo, OH, COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH or —C₁-C₆ alkyl-NH₂;
R₁ₐ, R_{1b}, R_{1c}, R_{1d}, R₁ₑ, R_{1f}, R_{1g}, and R₁ₕ are independently selected from CN, halo, OH, COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH, —C₁-C₆ alkyl-CN, —S(O)₂—C₁-C₆ alkyl, 4-7-membered heterocyclyl, oxo, —C(═O)—NH₂, —C(═O)—O—C₁-C₆ alkyl, or —C₁-C₆ alkyl-NH₂; or
R₁ₐ and R_{1b}, R_{1c} and R_{1d}, R₁ₑ and R_{1f} and/or R_{1g} and R₁ₕ, together with the carbon atom to which they are attached form a C₃-C₆ cycloalkyl; or
any two of R₁ₐ, R_{1b}, R_{1c}, R_{1d}, R₁ₑ, R_{1f}, R_{1g}, and R₁ₕ on different carbon atoms are linked together through a —(CH₂)ₙ— linker to form a bridged ring, wherein n is 1, 2 or 3.

13. (canceled)

14. The compound of claim 6, wherein R₁ is 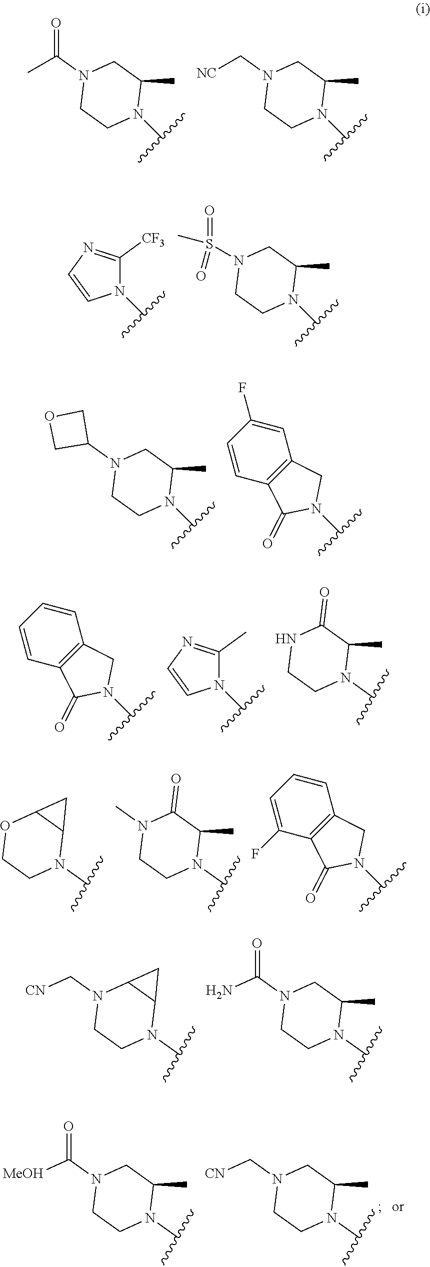 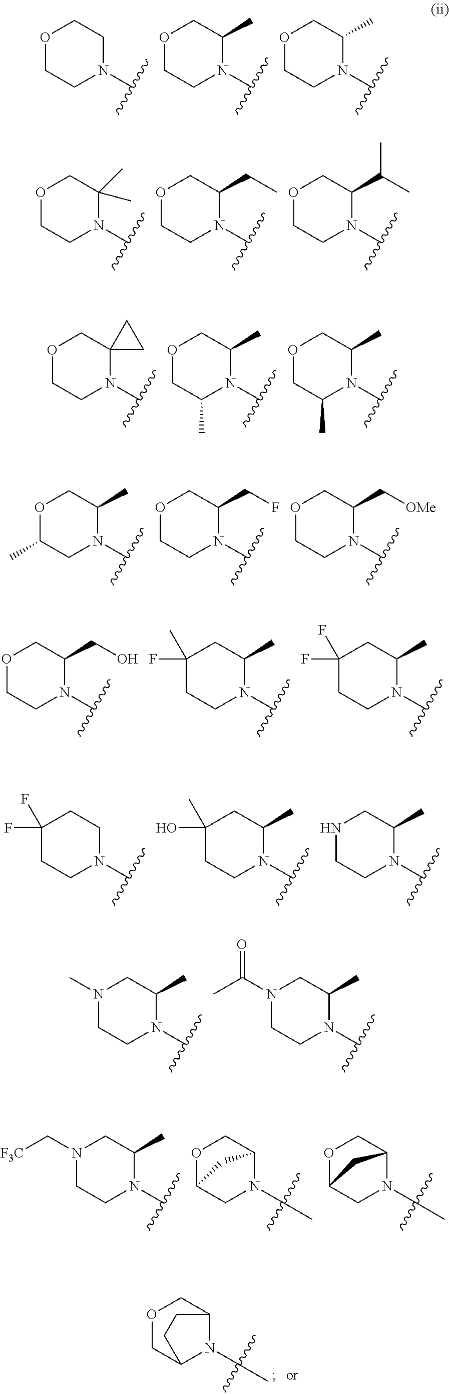 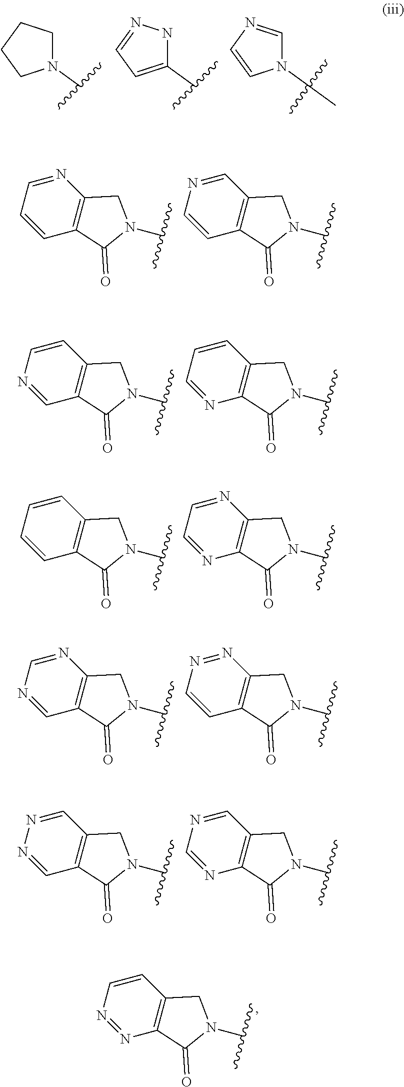
optionally substituted with one or more groups selected from CN, halo, OH, —COOH, NH₂, C₁-C₆ alkyl, C₁-C₆ haloalkyl, C₁-C₆ alkoxyl, —C(═O)—C₁-C₆ alkyl, —C₁-C₆ alkyl-OC₁-C₆ alkyl, —C₁-C₆ alkyl-OH, and —C₁-C₆ alkyl-NH₂; or

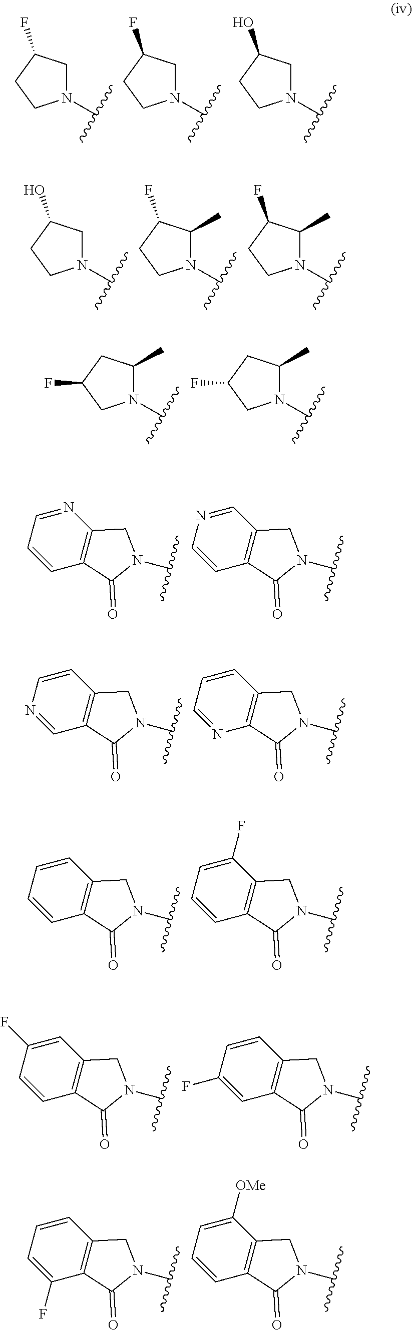

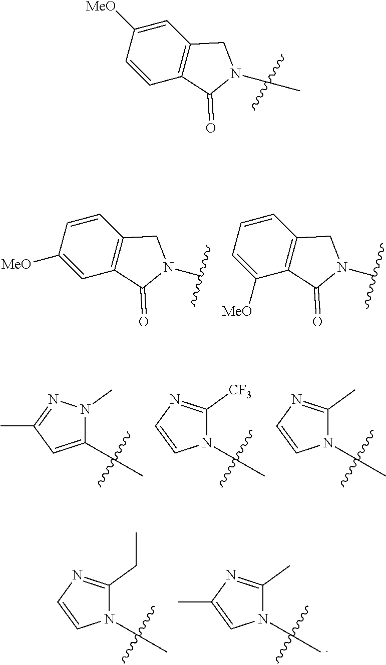

15-17. (canceled)

18. The compound of claim 1, wherein R₂ and R₃ are both methyl.

19. The compound of claim 1, wherein W is CR^{a}R^{b}, and R^{a} and R^{b} are each independently selected from H, halo, C₁-C₆ alkyl, OH or C₃-C₆ cycloalkyl.

20. The compound of claim 1, wherein R₄ is 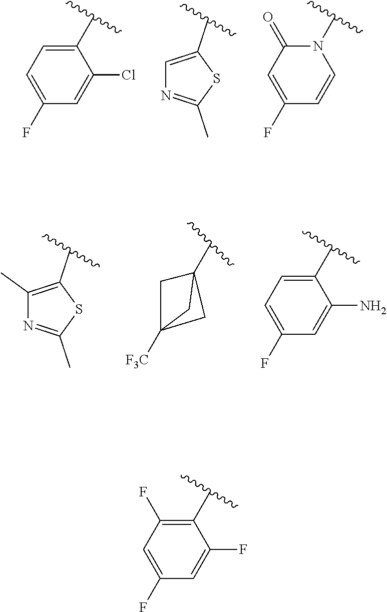
(i) R₄ is 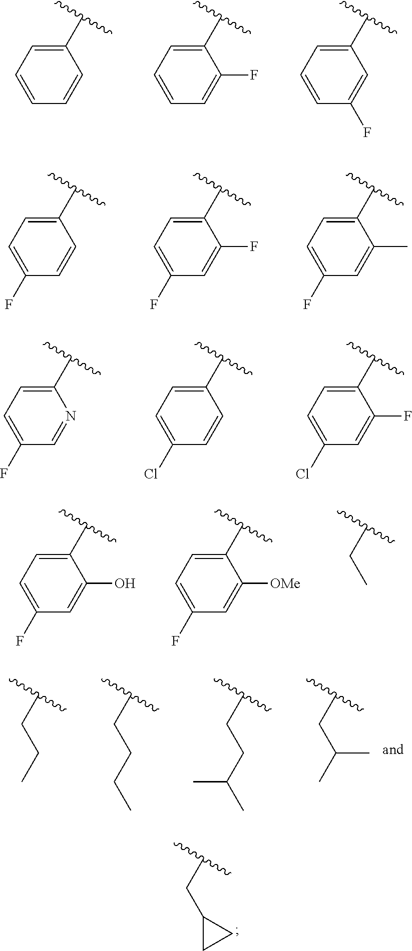
or
(ii) R₄ is 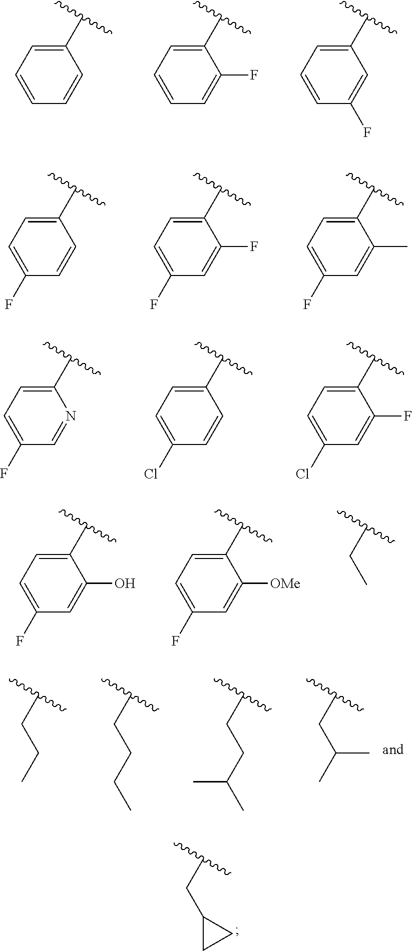
or
(iii) —W—R₄ is a moiety selected from:

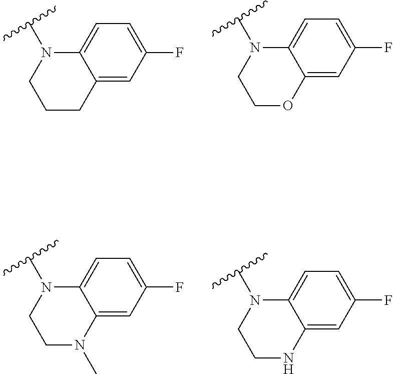 [SMILES: *N1CCCc2cc(F)ccc21 ; *N1CCOc2cc(F)ccc21 ; *N1CCN(C)c2cc(F)ccc21 ; *N1CCNc2cc(F)ccc21]

21-22. (canceled)

23. The compound of claim 1, wherein R₅ and R₆ are both hydrogen.

24. (canceled)

25. A compound selected from: 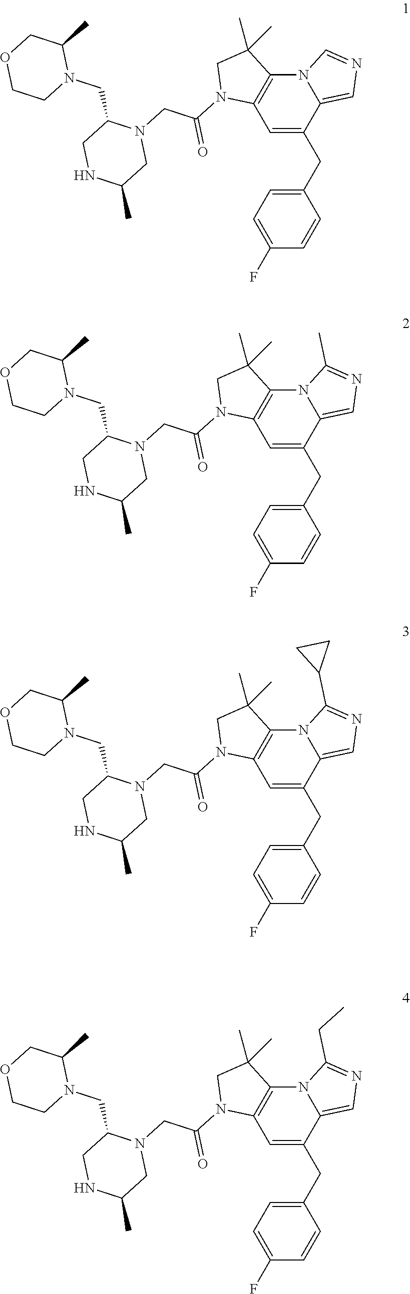 [SMILES: C[C@@H]1CN(CC(=O)N2CC(C)(C)c3c2cc(Cc2ccc(F)cc2)c2cncn32)[C@@H](CN2CCOC[C@H]2C)CN1 ; Cc1ncc2c(Cc3ccc(F)cc3)cc3c(n12)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2cnc(C4CC4)n32)[C@@H](CN2CCOC[C@H]2C)CN1 ; CCc1ncc2c(Cc3ccc(F)cc3)cc3c(n12)C(C)(C)CN3C(=O)CN1C[C@@H](C)NC[C@@H]1CN1CCOC[C@H]1C] 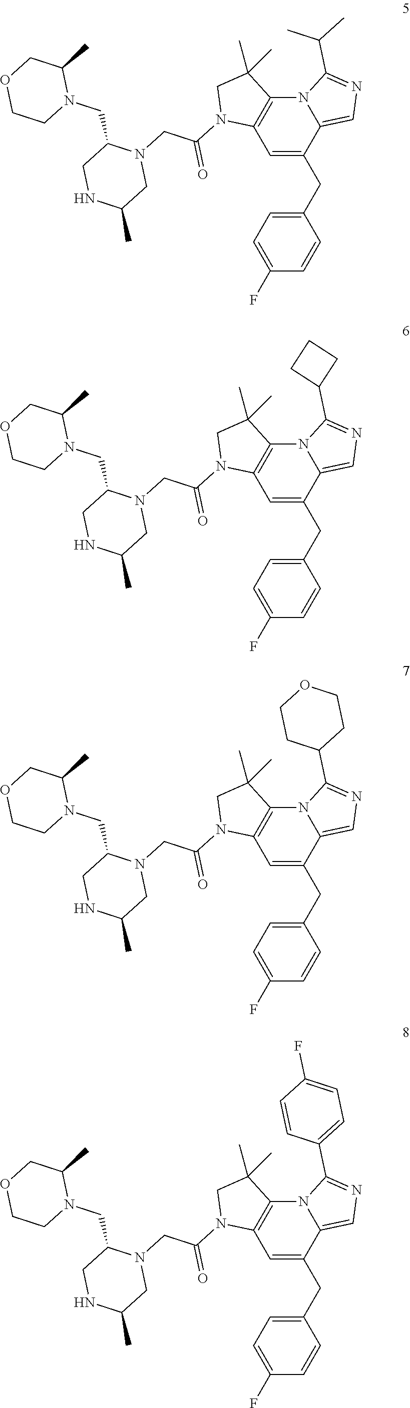 [SMILES: CC(C)c1ncc2c(Cc3ccc(F)cc3)cc3c(n12)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2cnc(C4CCC4)n32)[C@@H](CN2CCOC[C@H]2C)CN1 ; C[C@@H]1CN(CC(=O)N2CC(C)(C)c3c2cc(Cc2ccc(F)cc2)c2cnc(C4CCOCC4)n32)[C@@H](CN2CCOC[C@H]2C)CN1 ; C[C@@H]1CN(CC(=O)N2CC(C)(C)c3c2cc(Cc2ccc(F)cc2)c2cnc(-c4ccc(F)cc4)n32)[C@@H](CN2CCOC[C@H]2C)CN1] 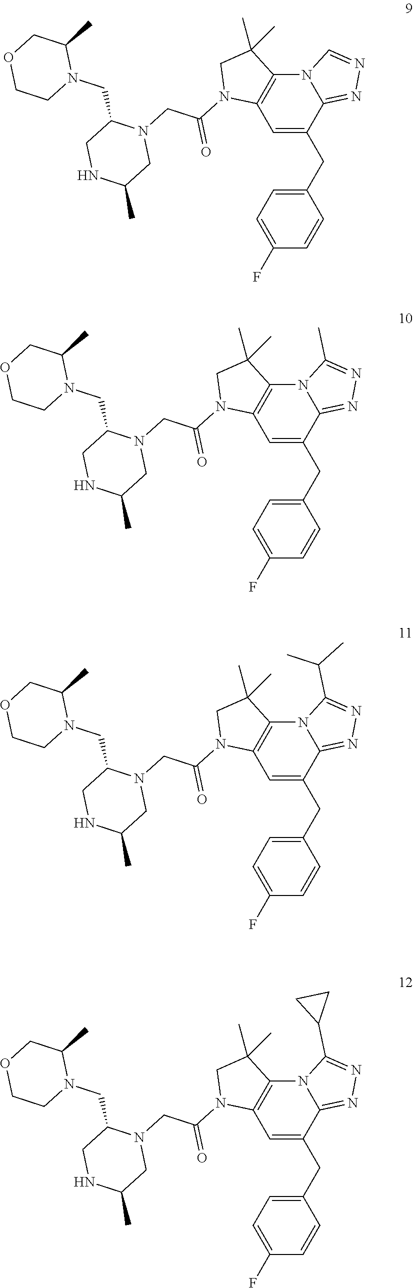 [SMILES: C[C@@H]1CN(CC(=O)N2CC(C)(C)c3c2cc(Cc2ccc(F)cc2)c2nncn32)[C@@H](CN2CCOC[C@H]2C)CN1 ; Cc1nnc2c(Cc3ccc(F)cc3)cc3c(n12)C(C)(C)CN3C(=O)CN1C[C@@H](C)NC[C@@H]1CN1CCOC[C@H]1C ; CC(C)c1nnc2c(Cc3ccc(F)cc3)cc3c(n12)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2nnc(C4CC4)n32)[C@@H](CN2CCOC[C@H]2C)CN1] 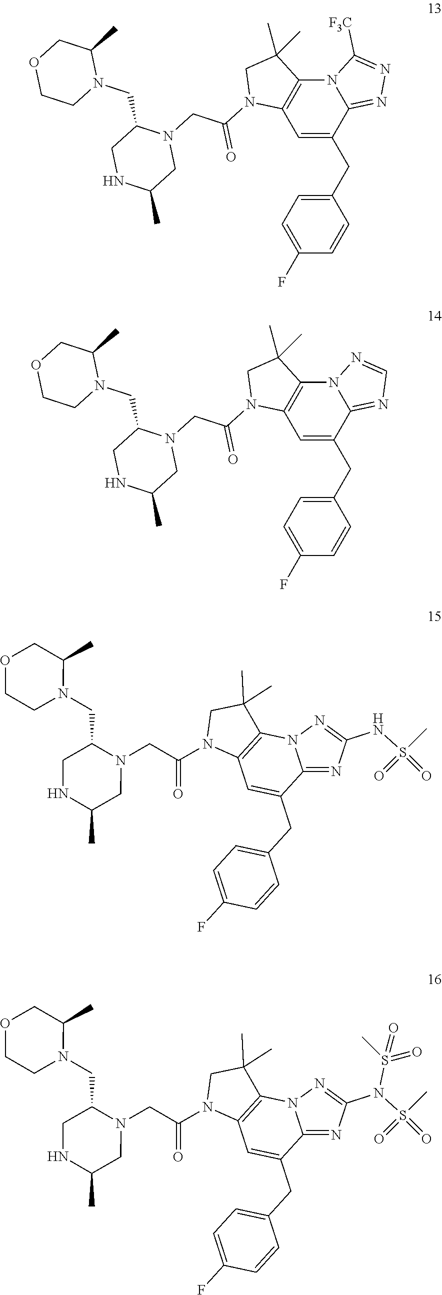 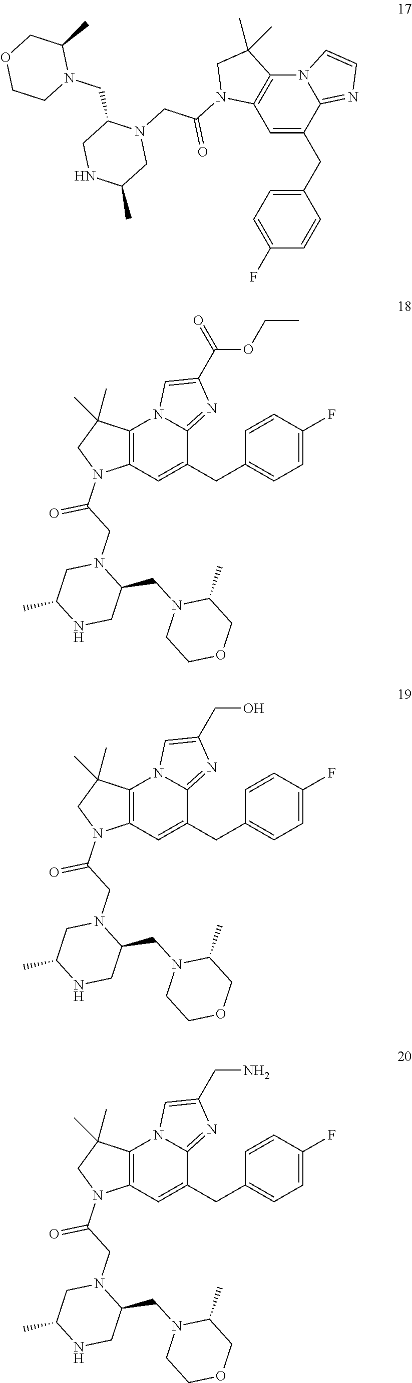 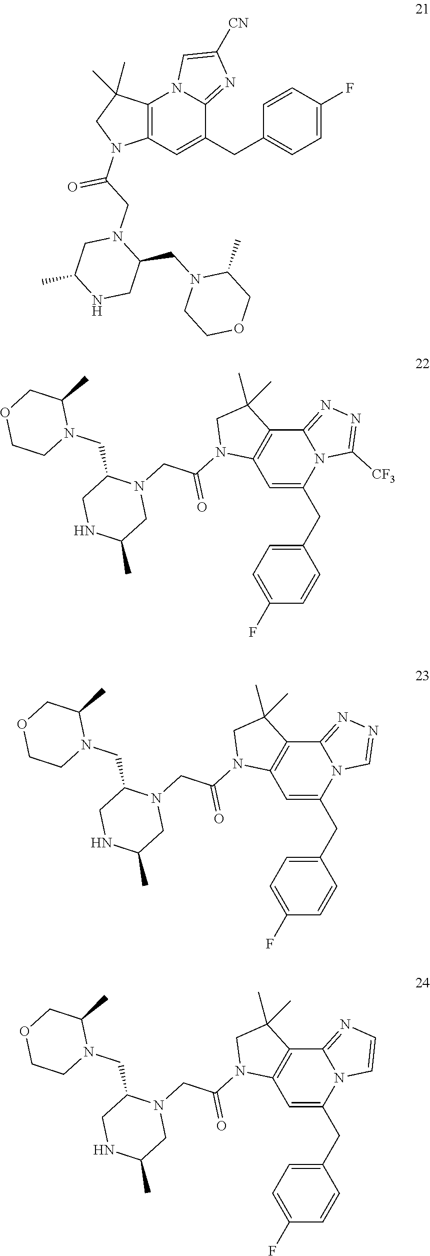 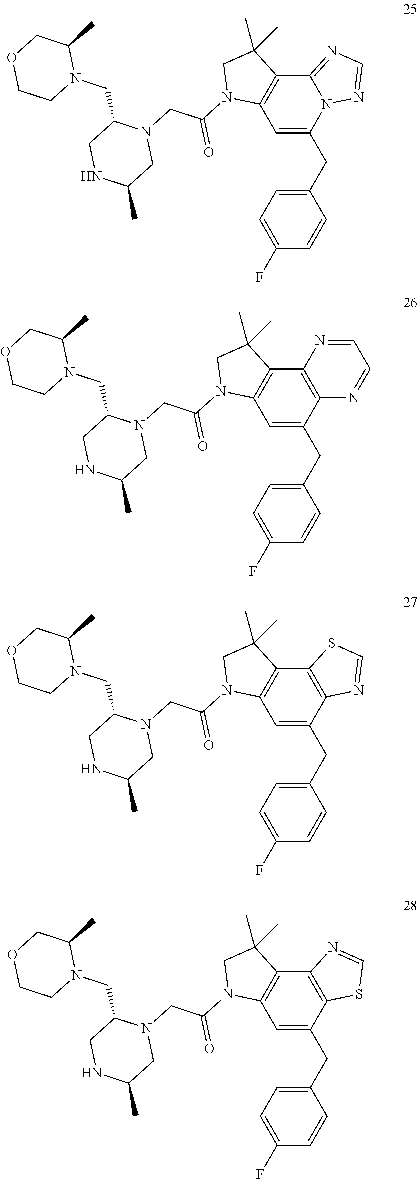 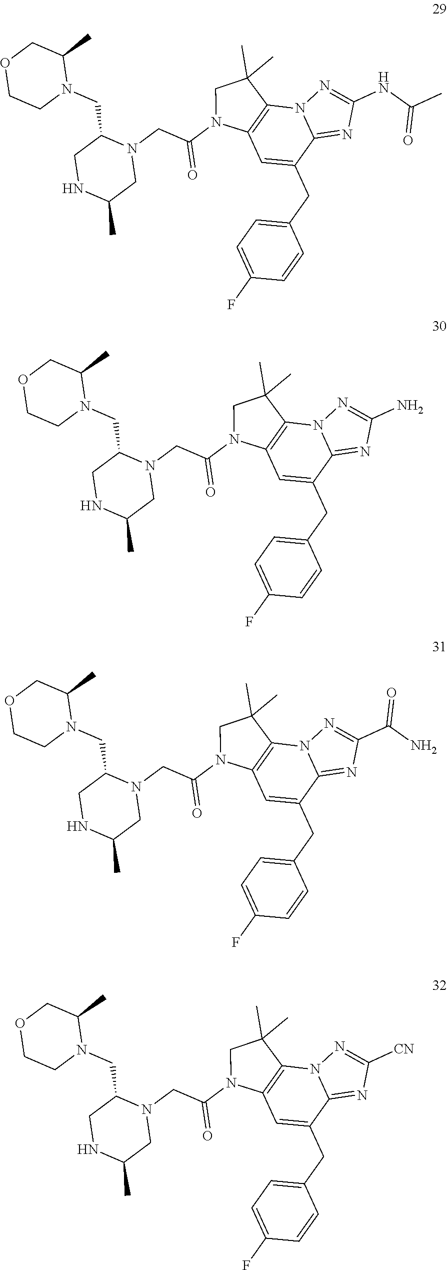 [SMILES: CC(=O)Nc1nc2c(Cc3ccc(F)cc3)cc3c(n2n1)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2nc(N)nn32)[C@@H](CN2CCOC[C@H]2C)CN1 ; C[C@@H]1CN(CC(=O)N2CC(C)(C)c3c2cc(Cc2ccc(F)cc2)c2nc(C(N)=O)nn32)[C@@H](CN2CCOC[C@H]2C)CN1 ; C[C@@H]1CN(CC(=O)N2CC(C)(C)c3c2cc(Cc2ccc(F)cc2)c2nc(C#N)nn32)[C@@H](CN2CCOC[C@H]2C)CN1] 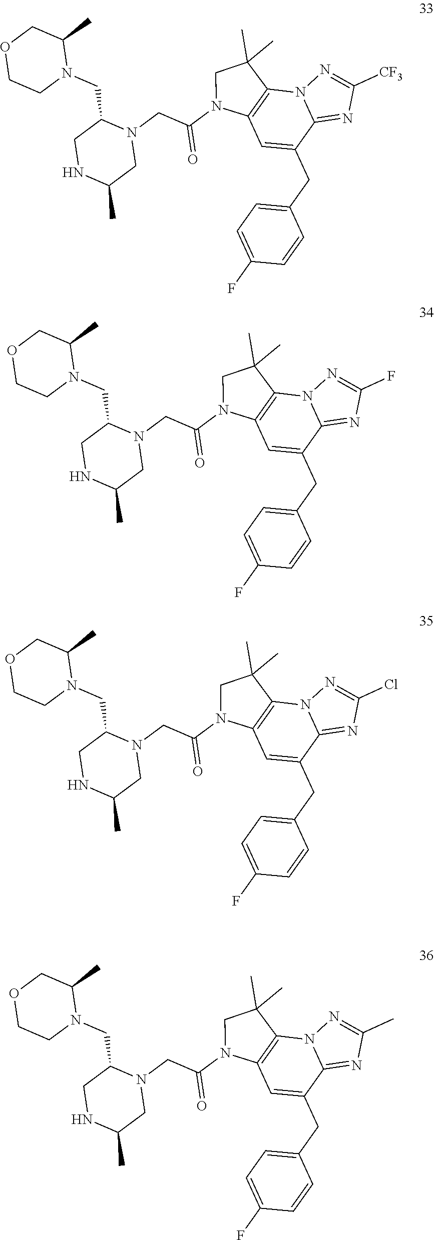 [SMILES: C[C@@H]1CN(CC(=O)N2CC(C)(C)c3c2cc(Cc2ccc(F)cc2)c2nc(C(F)(F)F)nn32)[C@@H](CN2CCOC[C@H]2C)CN1 ; C[C@@H]1CN(CC(=O)N2CC(C)(C)c3c2cc(Cc2ccc(F)cc2)c2nc(F)nn32)[C@@H](CN2CCOC[C@H]2C)CN1 ; C[C@@H]1CN(CC(=O)N2CC(C)(C)c3c2cc(Cc2ccc(F)cc2)c2nc(Cl)nn32)[C@@H](CN2CCOC[C@H]2C)CN1 ; Cc1nc2c(Cc3ccc(F)cc3)cc3c(n2n1)C(C)(C)CN3C(=O)CN1C[C@@H](C)NC[C@@H]1CN1CCOC[C@H]1C] 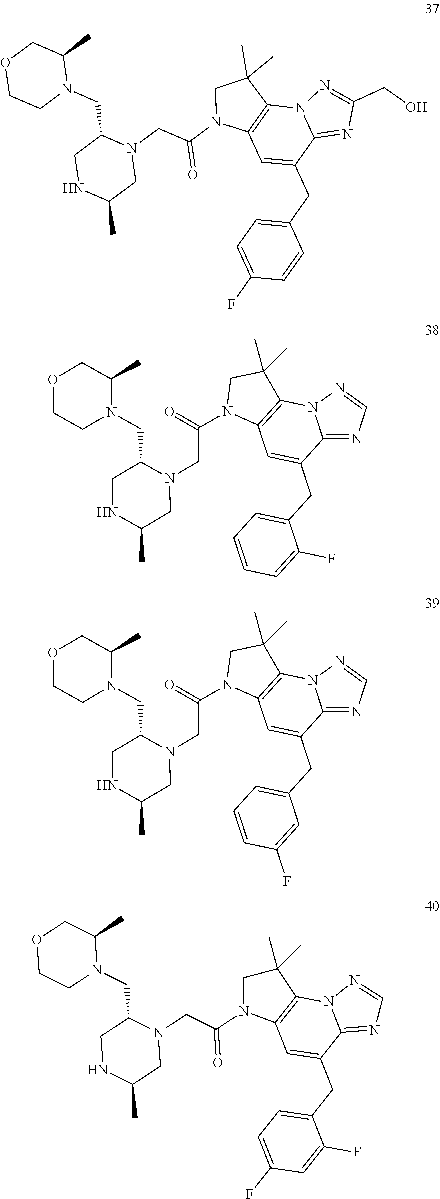 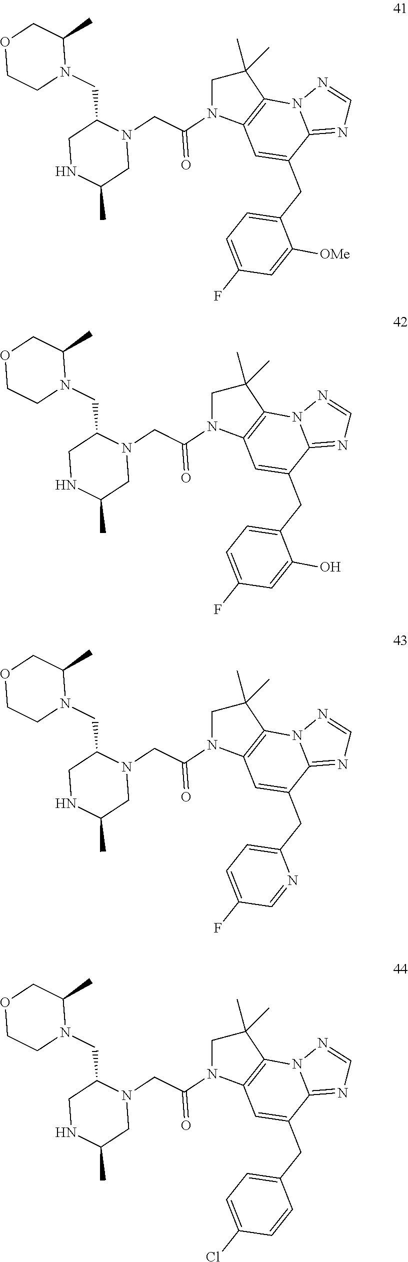 [SMILES: COc1cc(F)ccc1Cc1cc2c(n3ncnc13)C(C)(C)CN2C(=O)CN1C[C@@H](C)NC[C@@H]1CN1CCOC[C@H]1C ; C[C@@H]1CN(CC(=O)N2CC(C)(C)c3c2cc(Cc2ccc(F)cc2O)c2ncnn32)[C@@H](CN2CCOC[C@H]2C)CN1 ; C[C@@H]1CN(CC(=O)N2CC(C)(C)c3c2cc(Cc2ccc(F)cn2)c2ncnn32)[C@@H](CN2CCOC[C@H]2C)CN1 ; C[C@@H]1CN(CC(=O)N2CC(C)(C)c3c2cc(Cc2ccc(Cl)cc2)c2ncnn32)[C@@H](CN2CCOC[C@H]2C)CN1] 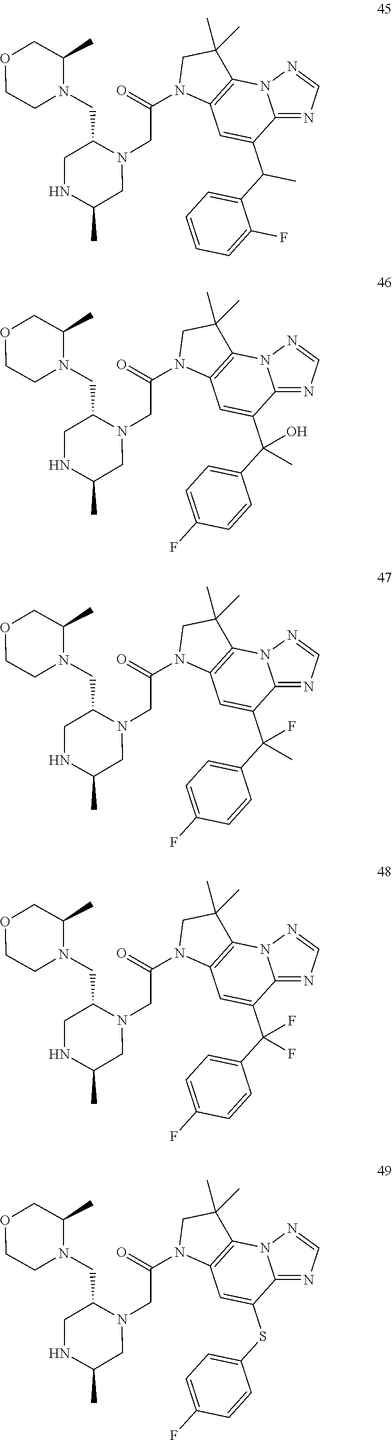 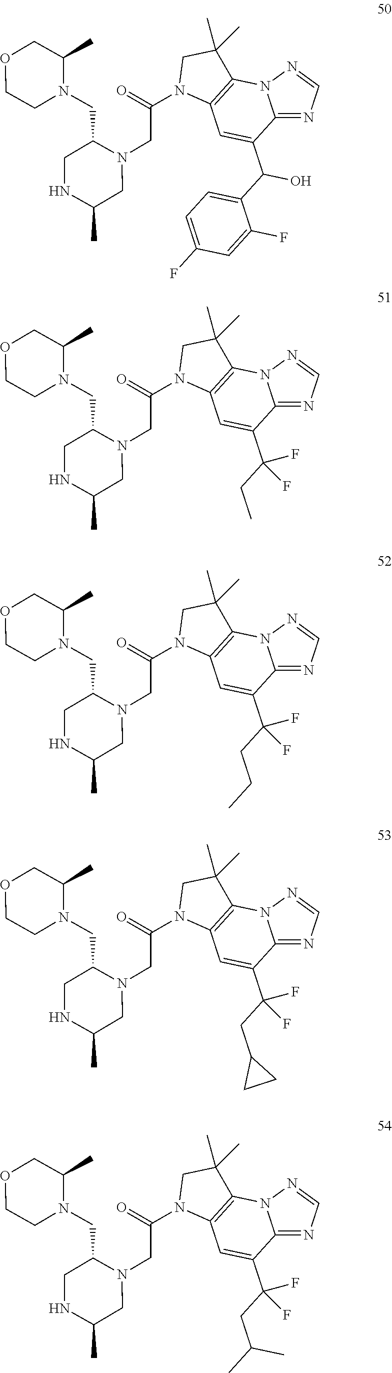 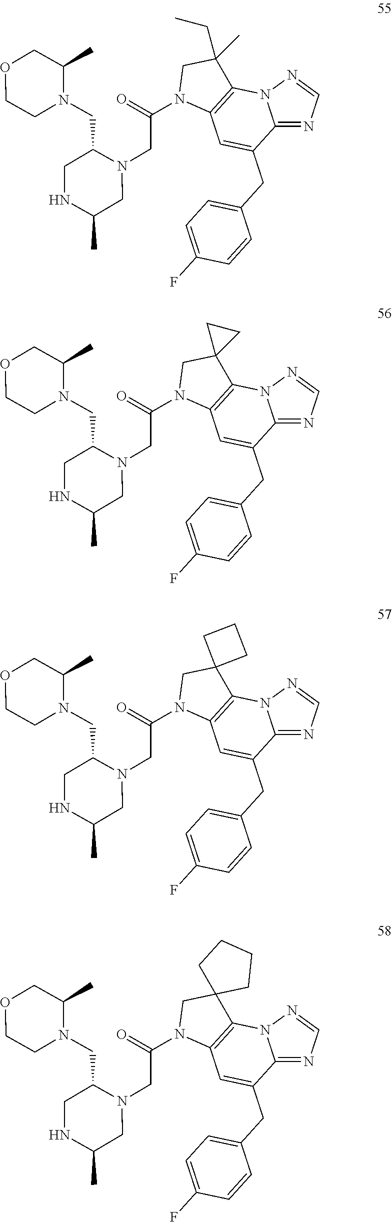 [SMILES: CCC1(C)CN(C(=O)CN2C[C@@H](C)NC[C@@H]2CN2CCOC[C@H]2C)c2cc(Cc3ccc(F)cc3)c3ncnn3c21 ; C[C@@H]1CN(CC(=O)N2CC3(CC3)c3c2cc(Cc2ccc(F)cc2)c2ncnn32)[C@@H](CN2CCOC[C@H]2C)CN1 ; C[C@@H]1CN(CC(=O)N2CC3(CCC3)c3c2cc(Cc2ccc(F)cc2)c2ncnn32)[C@@H](CN2CCOC[C@H]2C)CN1 ; C[C@@H]1CN(CC(=O)N2CC3(CCCC3)c3c2cc(Cc2ccc(F)cc2)c2ncnn32)[C@@H](CN2CCOC[C@H]2C)CN1] 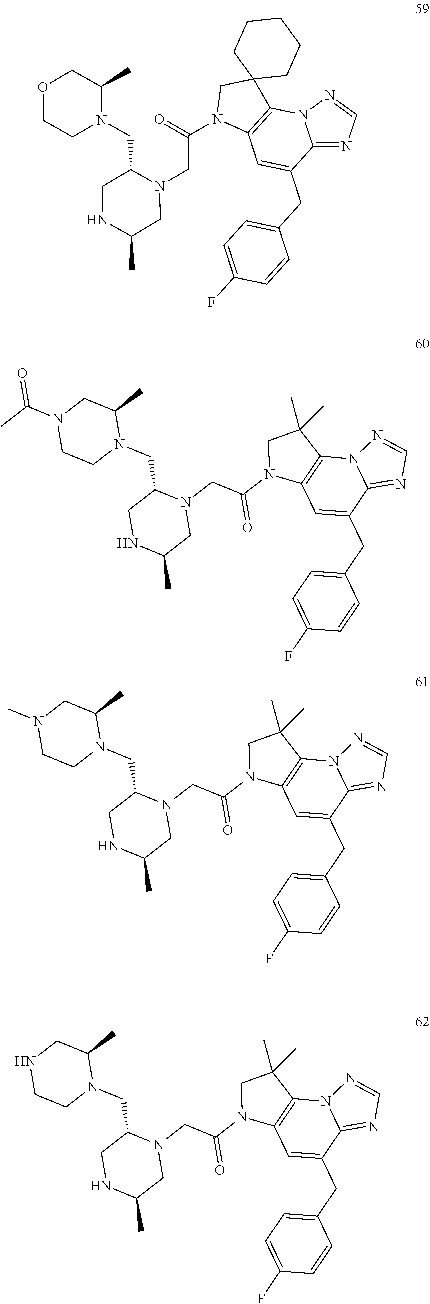 [SMILES: C[C@@H]1CN(CC(=O)N2CC3(CCCCC3)c3c2cc(Cc2ccc(F)cc2)c2ncnn32)[C@@H](CN2CCOC[C@H]2C)CN1 ; CC(=O)N1CCN(C[C@H]2CN[C@H](C)CN2CC(=O)N2CC(C)(C)c3c2cc(Cc2ccc(F)cc2)c2ncnn32)[C@H](C)C1 ; C[C@@H]1CN(CC(=O)N2CC(C)(C)c3c2cc(Cc2ccc(F)cc2)c2ncnn32)[C@@H](CN2CCN(C)C[C@H]2C)CN1 ; C[C@@H]1CN(CC(=O)N2CC(C)(C)c3c2cc(Cc2ccc(F)cc2)c2ncnn32)[C@@H](CN2CCNC[C@H]2C)CN1] 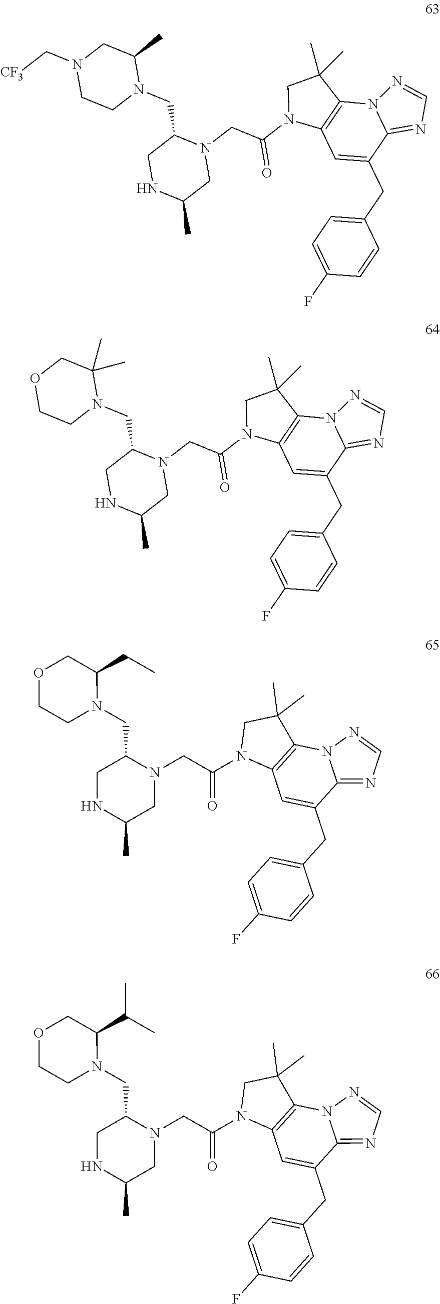 [SMILES: C[C@@H]1CN(CC(=O)N2CC(C)(C)c3c2cc(Cc2ccc(F)cc2)c2ncnn32)[C@@H](CN2CCN(CC(F)(F)F)C[C@H]2C)CN1 ; C[C@@H]1CN(CC(=O)N2CC(C)(C)c3c2cc(Cc2ccc(F)cc2)c2ncnn32)[C@@H](CN2CCOCC2(C)C)CN1 ; CC[C@@H]1COCCN1C[C@H]1CN[C@H](C)CN1CC(=O)N1CC(C)(C)c2c1cc(Cc1ccc(F)cc1)c1ncnn21 ; CC(C)[C@@H]1COCCN1C[C@H]1CN[C@H](C)CN1CC(=O)N1CC(C)(C)c2c1cc(Cc1ccc(F)cc1)c1ncnn21] 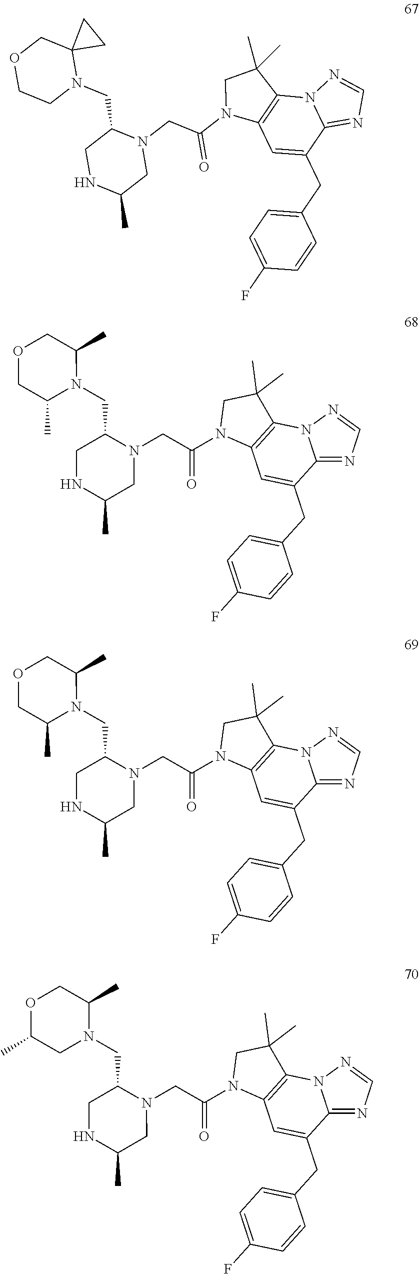 [SMILES: C[C@@H]1CN(CC(=O)N2CC(C)(C)c3c2cc(Cc2ccc(F)cc2)c2ncnn32)[C@@H](CN2CCOCC23CC3)CN1 ; C[C@@H]1CN(CC(=O)N2CC(C)(C)c3c2cc(Cc2ccc(F)cc2)c2ncnn32)[C@@H](CN2[C@H](C)COC[C@H]2C)CN1 ; C[C@@H]1CN(CC(=O)N2CC(C)(C)c3c2cc(Cc2ccc(F)cc2)c2ncnn32)[C@@H](CN2[C@H](C)COC[C@@H]2C)CN1 ; C[C@@H]1CN(CC(=O)N2CC(C)(C)c3c2cc(Cc2ccc(F)cc2)c2ncnn32)[C@@H](CN2C[C@H](C)OC[C@H]2C)CN1] 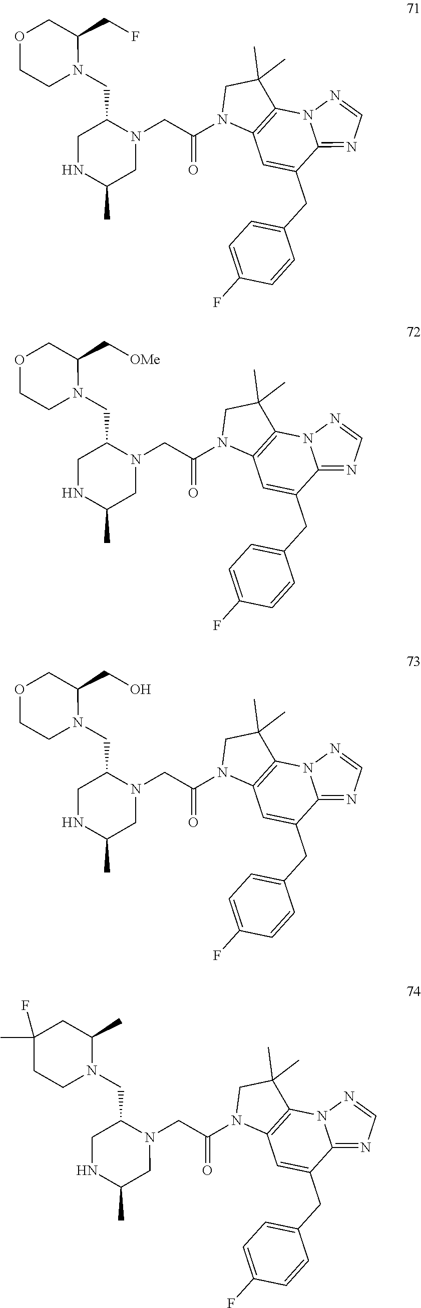 [SMILES: C[C@@H]1CN(CC(=O)N2CC(C)(C)c3c2cc(Cc2ccc(F)cc2)c2ncnn32)[C@@H](CN2CCOC[C@H]2CF)CN1 ; COC[C@@H]1COCCN1C[C@H]1CN[C@H](C)CN1CC(=O)N1CC(C)(C)c2c1cc(Cc1ccc(F)cc1)c1ncnn21 ; C[C@@H]1CN(CC(=O)N2CC(C)(C)c3c2cc(Cc2ccc(F)cc2)c2ncnn32)[C@@H](CN2CCOC[C@H]2CO)CN1 ; C[C@@H]1CN(CC(=O)N2CC(C)(C)c3c2cc(Cc2ccc(F)cc2)c2ncnn32)[C@@H](CN2CCC(C)(F)C[C@H]2C)CN1] 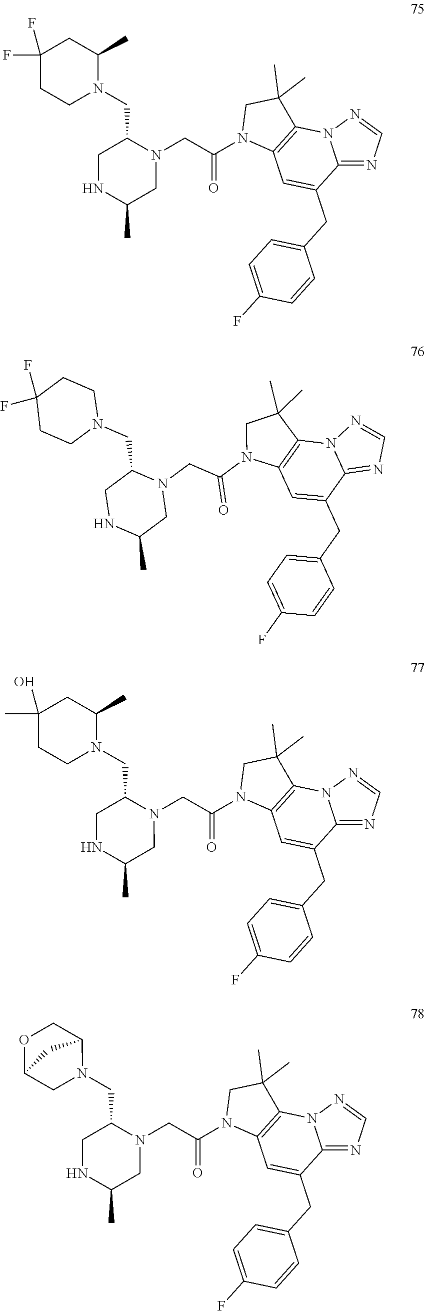 [SMILES: C[C@@H]1CN(CC(=O)N2CC(C)(C)c3c2cc(Cc2ccc(F)cc2)c2ncnn32)[C@@H](CN2CCC(F)(F)C[C@H]2C)CN1 ; C[C@@H]1CN(CC(=O)N2CC(C)(C)c3c2cc(Cc2ccc(F)cc2)c2ncnn32)[C@@H](CN2CCC(F)(F)CC2)CN1 ; C[C@@H]1CN(CC(=O)N2CC(C)(C)c3c2cc(Cc2ccc(F)cc2)c2ncnn32)[C@@H](CN2CCC(C)(O)C[C@H]2C)CN1 ; C[C@@H]1CN(CC(=O)N2CC(C)(C)c3c2cc(Cc2ccc(F)cc2)c2ncnn32)[C@@H](CN2C[C@@H]3C[C@H]2CO3)CN1] 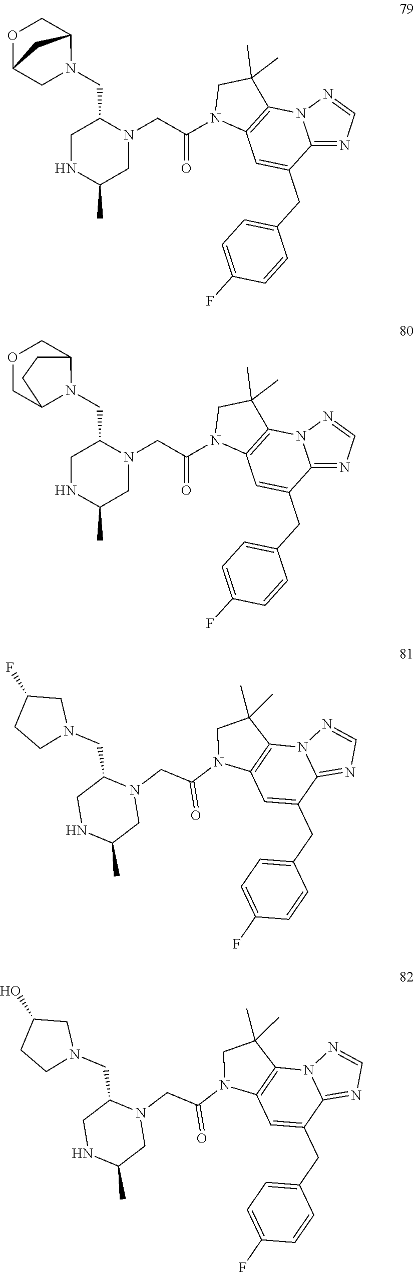 [SMILES: C[C@@H]1CN(CC(=O)N2CC(C)(C)c3c2cc(Cc2ccc(F)cc2)c2ncnn32)[C@@H](CN2C[C@H]3C[C@@H]2CO3)CN1 ; C[C@@H]1CN(CC(=O)N2CC(C)(C)c3c2cc(Cc2ccc(F)cc2)c2ncnn32)[C@@H](CN2C3CCC2COC3)CN1 ; C[C@@H]1CN(CC(=O)N2CC(C)(C)c3c2cc(Cc2ccc(F)cc2)c2ncnn32)[C@@H](CN2CC[C@H](F)C2)CN1 ; C[C@@H]1CN(CC(=O)N2CC(C)(C)c3c2cc(Cc2ccc(F)cc2)c2ncnn32)[C@@H](CN2CC[C@H](O)C2)CN1] 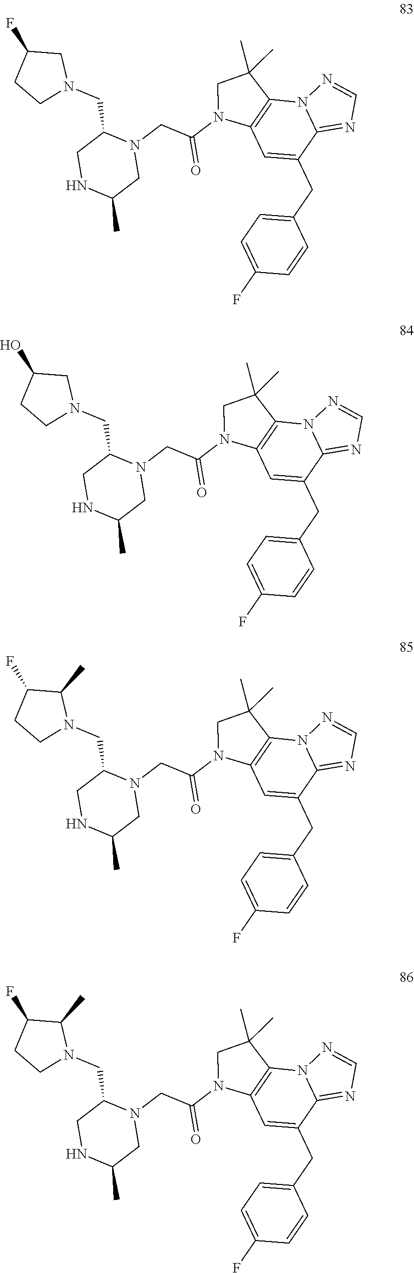 [SMILES: C[C@@H]1CN(CC(=O)N2CC(C)(C)c3c2cc(Cc2ccc(F)cc2)c2ncnn32)[C@@H](CN2CC[C@@H](F)C2)CN1 ; C[C@@H]1CN(CC(=O)N2CC(C)(C)c3c2cc(Cc2ccc(F)cc2)c2ncnn32)[C@@H](CN2CC[C@@H](O)C2)CN1 ; C[C@@H]1CN(CC(=O)N2CC(C)(C)c3c2cc(Cc2ccc(F)cc2)c2ncnn32)[C@@H](CN2CC[C@H](F)[C@H]2C)CN1 ; C[C@@H]1CN(CC(=O)N2CC(C)(C)c3c2cc(Cc2ccc(F)cc2)c2ncnn32)[C@@H](CN2CC[C@@H](F)[C@H]2C)CN1] 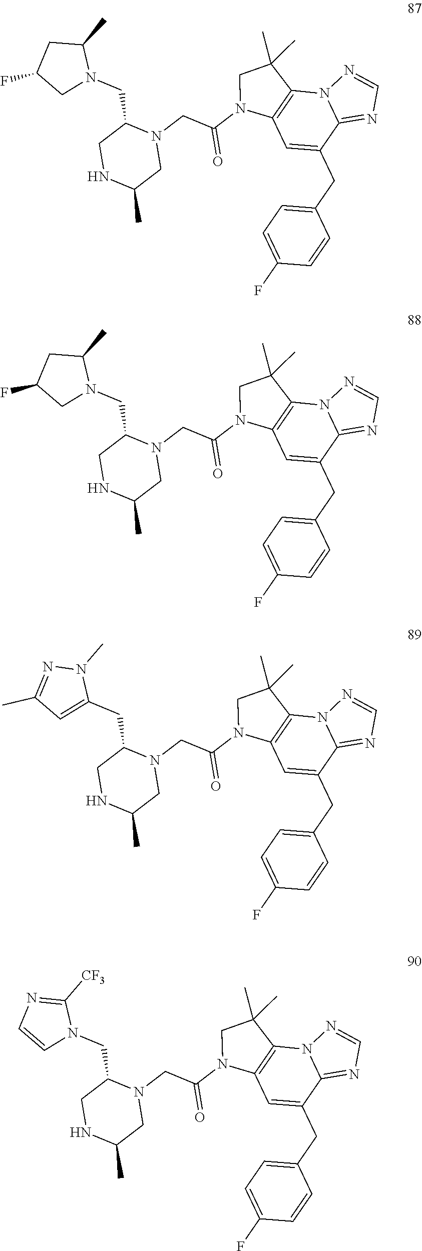 [SMILES: C[C@@H]1CN(CC(=O)N2CC(C)(C)c3c2cc(Cc2ccc(F)cc2)c2ncnn32)[C@@H](CN2C[C@H](F)C[C@H]2C)CN1 ; C[C@@H]1CN(CC(=O)N2CC(C)(C)c3c2cc(Cc2ccc(F)cc2)c2ncnn32)[C@@H](CN2C[C@@H](F)C[C@H]2C)CN1 ; Cc1cc(C[C@H]2CN[C@H](C)CN2CC(=O)N2CC(C)(C)c3c2cc(Cc2ccc(F)cc2)c2ncnn32)n(C)n1 ; C[C@@H]1CN(CC(=O)N2CC(C)(C)c3c2cc(Cc2ccc(F)cc2)c2ncnn32)[C@@H](Cn2ccnc2C(F)(F)F)CN1] 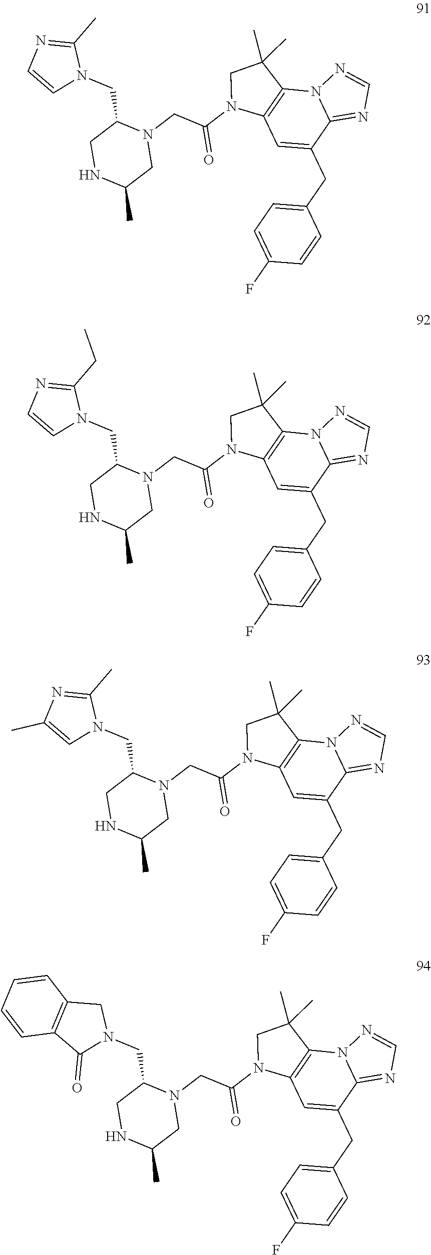 [SMILES: Cc1nccn1C[C@H]1CN[C@H](C)CN1CC(=O)N1CC(C)(C)c2c1cc(Cc1ccc(F)cc1)c1ncnn21 ; CCc1nccn1C[C@H]1CN[C@H](C)CN1CC(=O)N1CC(C)(C)c2c1cc(Cc1ccc(F)cc1)c1ncnn21 ; Cc1cn(C[C@H]2CN[C@H](C)CN2CC(=O)N2CC(C)(C)c3c2cc(Cc2ccc(F)cc2)c2ncnn32)c(C)n1 ; C[C@@H]1CN(CC(=O)N2CC(C)(C)c3c2cc(Cc2ccc(F)cc2)c2ncnn32)[C@@H](CN2Cc3ccccc3C2=O)CN1] 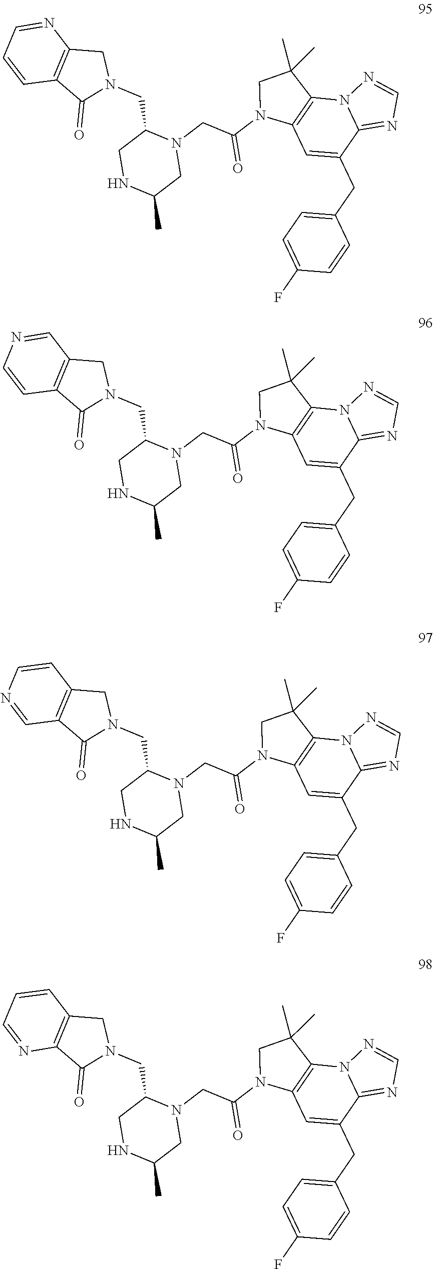 [SMILES: C[C@@H]1CN(CC(=O)N2CC(C)(C)c3c2cc(Cc2ccc(F)cc2)c2ncnn32)[C@@H](CN2Cc3ncccc3C2=O)CN1 ; C[C@@H]1CN(CC(=O)N2CC(C)(C)c3c2cc(Cc2ccc(F)cc2)c2ncnn32)[C@@H](CN2Cc3cnccc3C2=O)CN1 ; C[C@@H]1CN(CC(=O)N2CC(C)(C)c3c2cc(Cc2ccc(F)cc2)c2ncnn32)[C@@H](CN2Cc3ccncc3C2=O)CN1 ; C[C@@H]1CN(CC(=O)N2CC(C)(C)c3c2cc(Cc2ccc(F)cc2)c2ncnn32)[C@@H](CN2Cc3cccnc3C2=O)CN1] 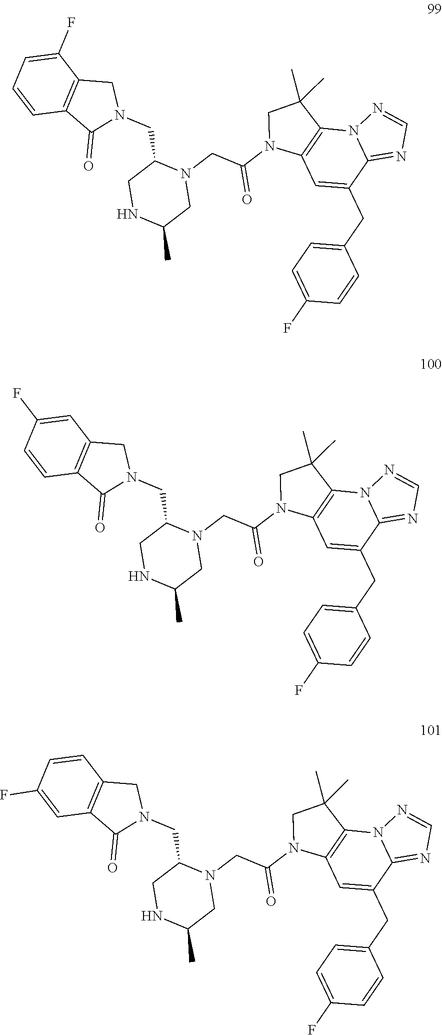 [SMILES: C[C@@H]1CN(CC(=O)N2CC(C)(C)c3c2cc(Cc2ccc(F)cc2)c2ncnn32)[C@@H](CN2Cc3c(F)cccc3C2=O)CN1 ; C[C@@H]1CN(CC(=O)N2CC(C)(C)c3c2cc(Cc2ccc(F)cc2)c2ncnn32)[C@@H](CN2Cc3cc(F)ccc3C2=O)CN1 ; C[C@@H]1CN(CC(=O)N2CC(C)(C)c3c2cc(Cc2ccc(F)cc2)c2ncnn32)[C@@H](CN2Cc3ccc(F)cc3C2=O)CN1] 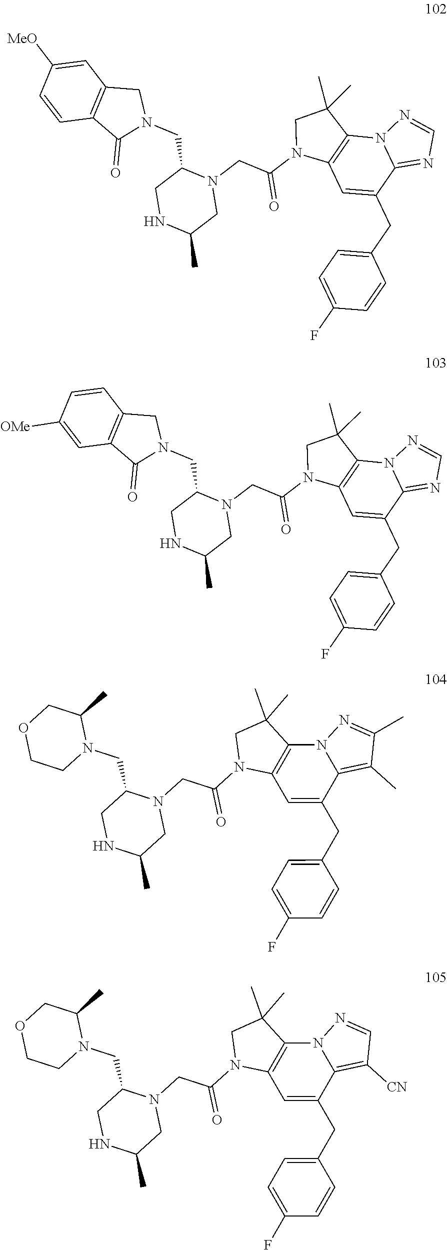 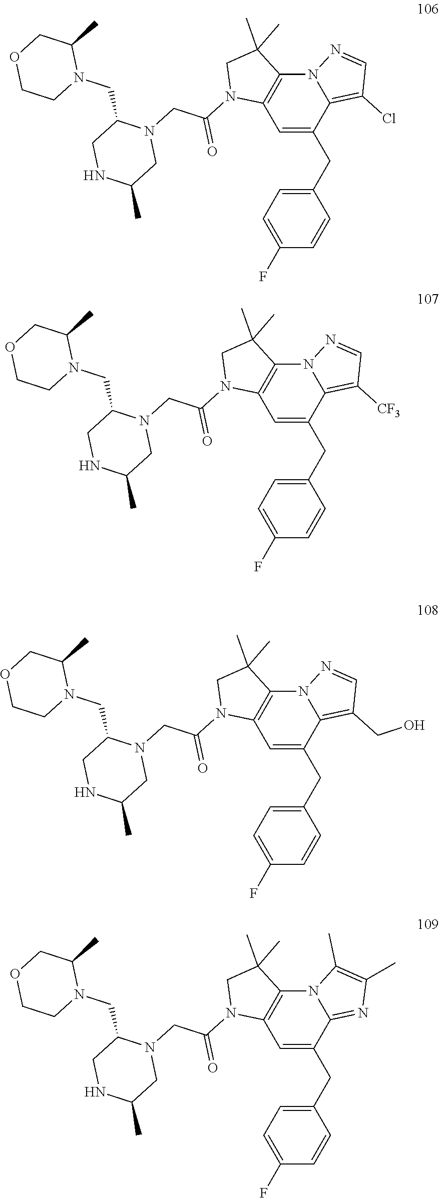 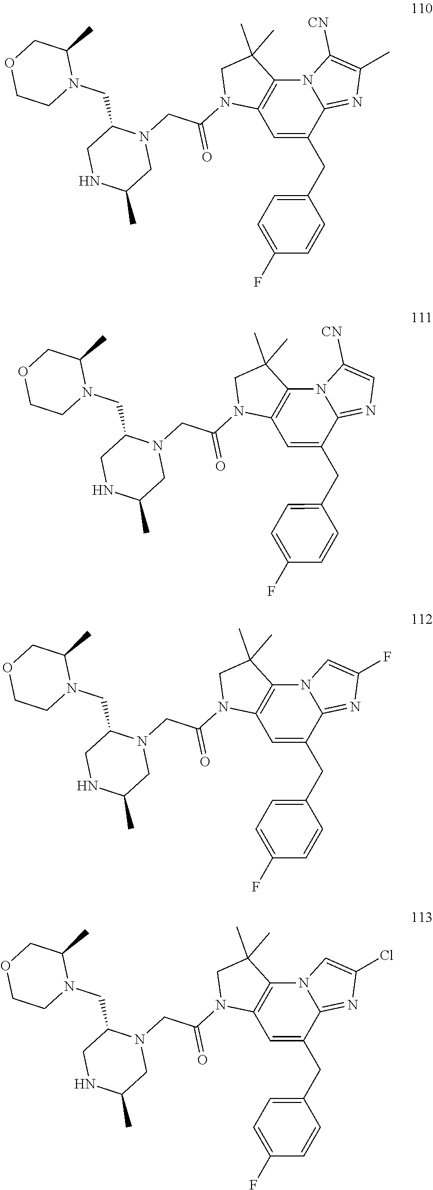 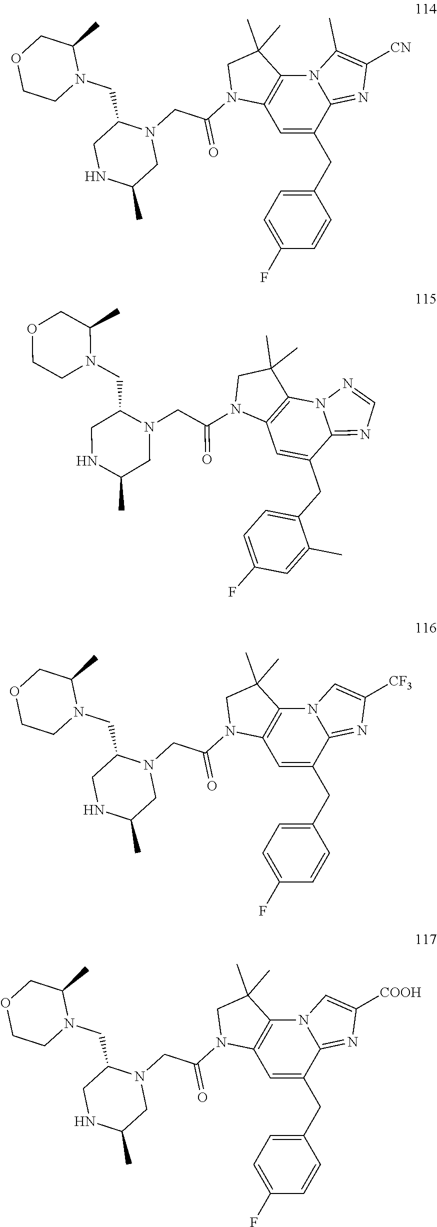 [SMILES: Cc1c(C#N)nc2c(Cc3ccc(F)cc3)cc3c(n12)C(C)(C)CN3C(=O)CN1C[C@@H](C)NC[C@@H]1CN1CCOC[C@H]1C ; Cc1cc(F)ccc1Cc1cc2c(n3ncnc13)C(C)(C)CN2C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2nc(C(F)(F)F)cn32)[C@@H](CN2CCOC[C@H]2C)CN1 ; C[C@@H]1CN(CC(=O)N2CC(C)(C)c3c2cc(Cc2ccc(F)cc2)c2nc(C(=O)O)cn32)[C@@H](CN2CCOC[C@H]2C)CN1] 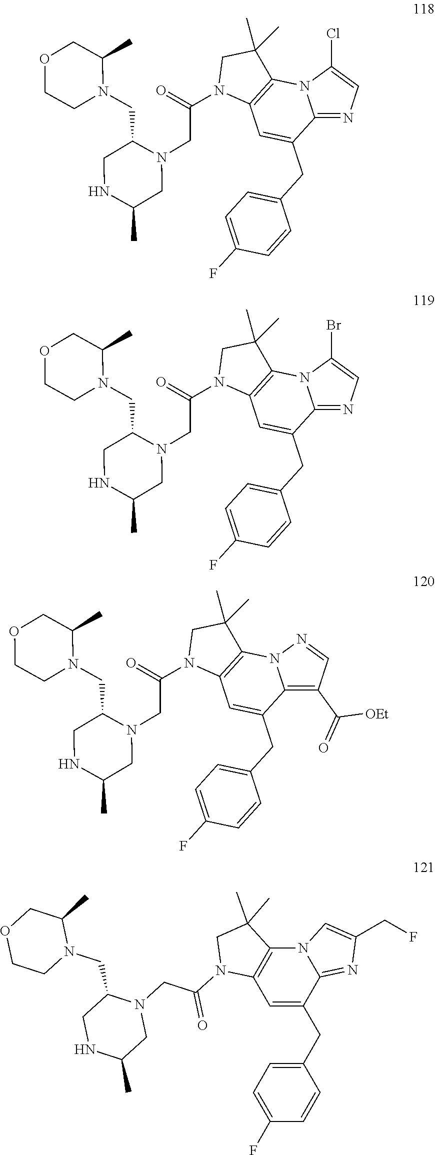 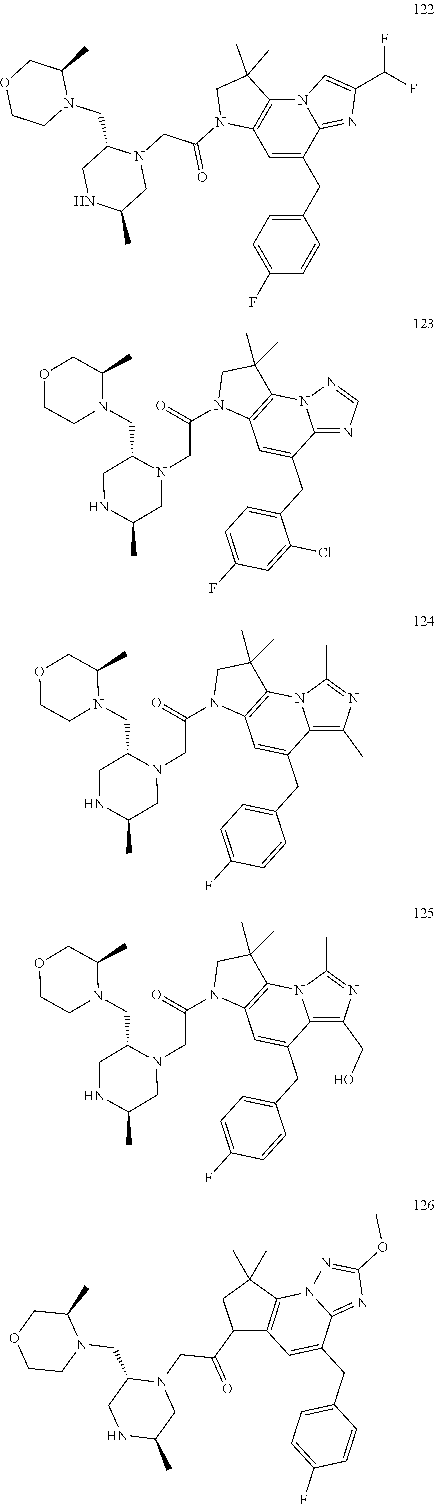 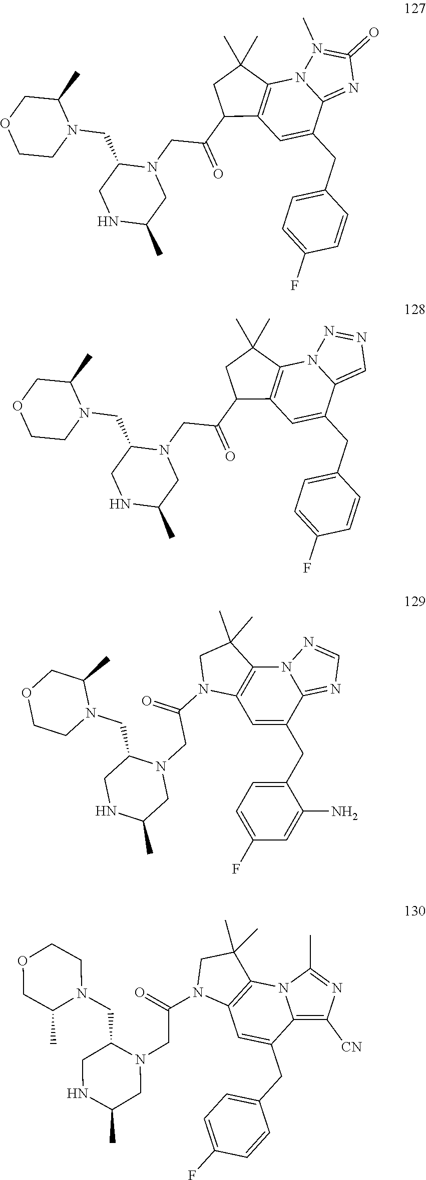 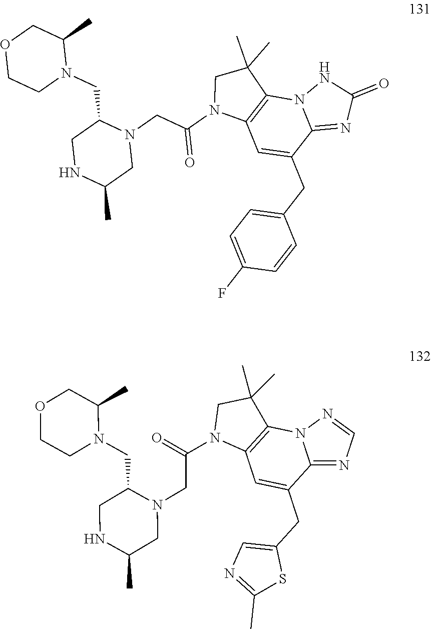 [SMILES: C[C@@H]1CN(CC(=O)N2CC(C)(C)c3c2cc(Cc2ccc(F)cc2)c2nc(=O)[nH]n32)[C@@H](CN2CCOC[C@H]2C)CN1 ; Cc1ncc(Cc2cc3c(n4ncnc24)C(C)(C)CN3C(=O)CN2C[C@@H](C)NC[C@@H]2CN2CCOC[C@H]2C)s1] 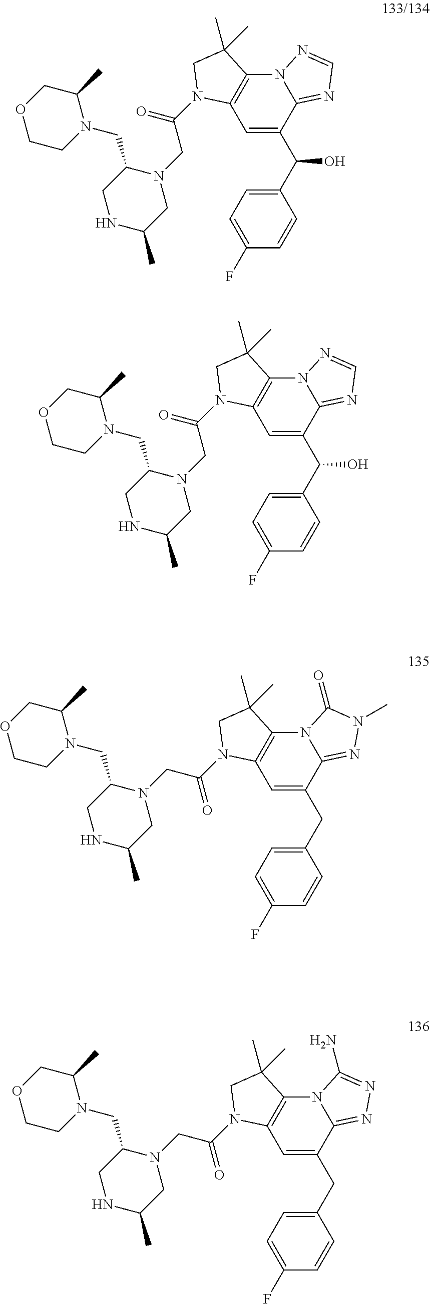 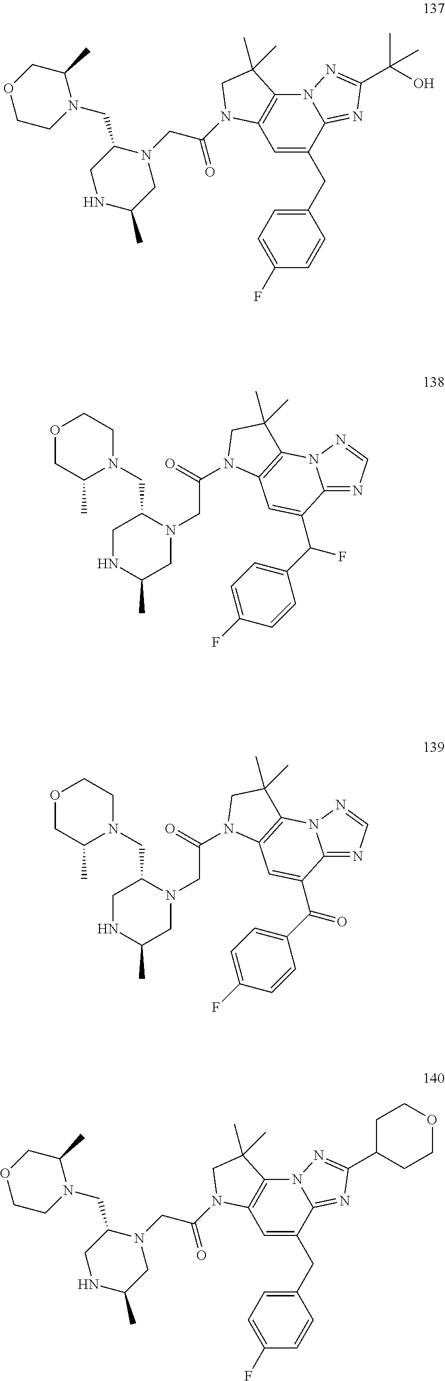 [SMILES: C[C@@H]1CN(CC(=O)N2CC(C)(C)c3c2cc(Cc2ccc(F)cc2)c2nc(C(C)(C)O)nn32)[C@@H](CN2CCOC[C@H]2C)CN1 ; C[C@@H]1CN(CC(=O)N2CC(C)(C)c3c2cc(C(F)c2ccc(F)cc2)c2ncnn32)[C@@H](CN2CCOC[C@H]2C)CN1 ; C[C@@H]1CN(CC(=O)N2CC(C)(C)c3c2cc(C(=O)c2ccc(F)cc2)c2ncnn32)[C@@H](CN2CCOC[C@H]2C)CN1 ; C[C@@H]1CN(CC(=O)N2CC(C)(C)c3c2cc(Cc2ccc(F)cc2)c2nc(C4CCOCC4)nn32)[C@@H](CN2CCOC[C@H]2C)CN1] 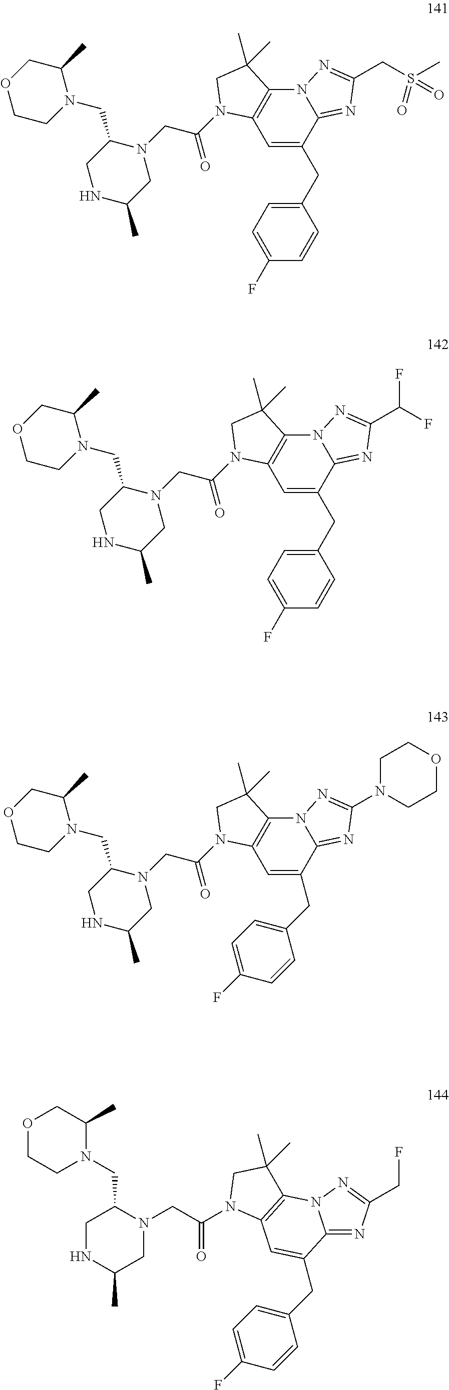 [SMILES: C[C@@H]1CN(CC(=O)N2CC(C)(C)c3c2cc(Cc2ccc(F)cc2)c2nc(CS(C)(=O)=O)nn32)[C@@H](CN2CCOC[C@H]2C)CN1 ; C[C@@H]1CN(CC(=O)N2CC(C)(C)c3c2cc(Cc2ccc(F)cc2)c2nc(C(F)F)nn32)[C@@H](CN2CCOC[C@H]2C)CN1 ; C[C@@H]1CN(CC(=O)N2CC(C)(C)c3c2cc(Cc2ccc(F)cc2)c2nc(N4CCOCC4)nn32)[C@@H](CN2CCOC[C@H]2C)CN1 ; C[C@@H]1CN(CC(=O)N2CC(C)(C)c3c2cc(Cc2ccc(F)cc2)c2nc(CF)nn32)[C@@H](CN2CCOC[C@H]2C)CN1] 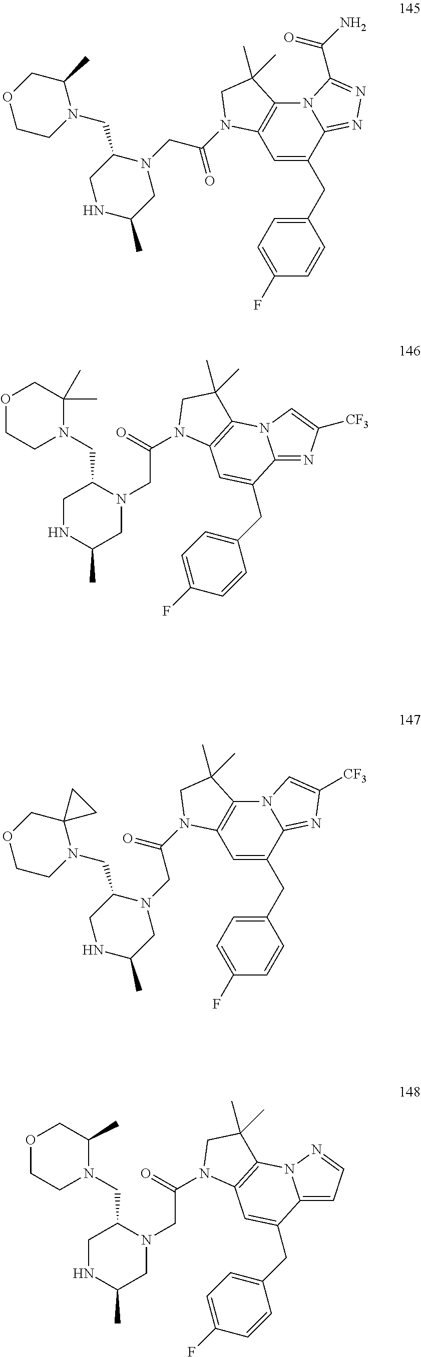 [SMILES: C[C@@H]1CN(CC(=O)N2CC(C)(C)c3c2cc(Cc2ccc(F)cc2)c2nnc(C(N)=O)n32)[C@@H](CN2CCOC[C@H]2C)CN1 ; C[C@@H]1CN(CC(=O)N2CC(C)(C)c3c2cc(Cc2ccc(F)cc2)c2nc(C(F)(F)F)cn32)[C@@H](CN2CCOCC2(C)C)CN1 ; C[C@@H]1CN(CC(=O)N2CC(C)(C)c3c2cc(Cc2ccc(F)cc2)c2nc(C(F)(F)F)cn32)[C@@H](CN2CCOCC23CC3)CN1 ; C[C@@H]1CN(CC(=O)N2CC(C)(C)c3c2cc(Cc2ccc(F)cc2)c2ccnn32)[C@@H](CN2CCOC[C@H]2C)CN1] 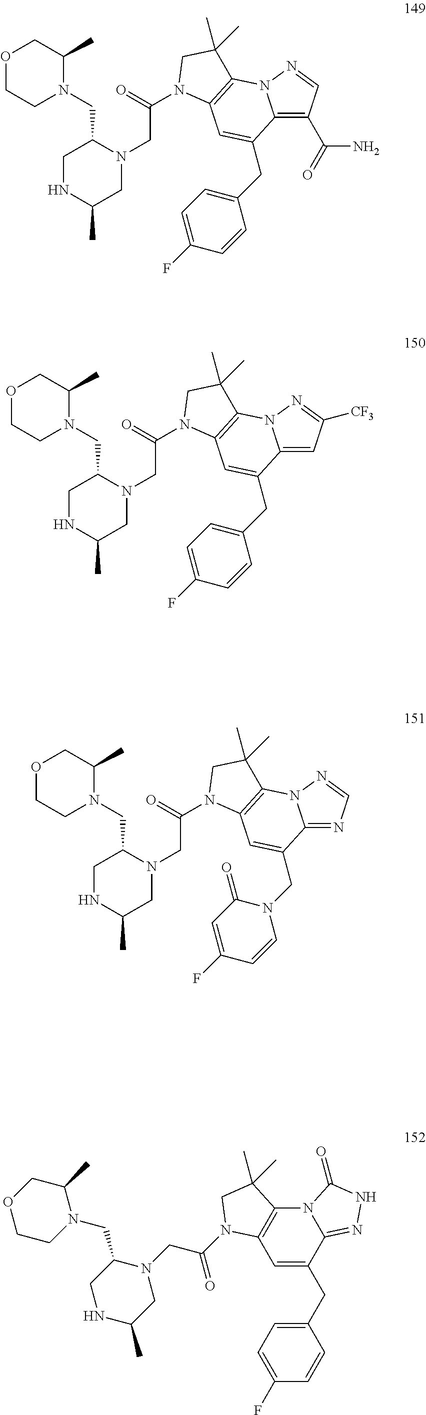 [SMILES: C[C@@H]1CN(CC(=O)N2CC(C)(C)c3c2cc(Cc2ccc(F)cc2)c2c(C(N)=O)cnn32)[C@@H](CN2CCOC[C@H]2C)CN1 ; C[C@@H]1CN(CC(=O)N2CC(C)(C)c3c2cc(Cc2ccc(F)cc2)c2cc(C(F)(F)F)nn32)[C@@H](CN2CCOC[C@H]2C)CN1 ; C[C@@H]1CN(CC(=O)N2CC(C)(C)c3c2cc(Cn2ccc(F)cc2=O)c2ncnn32)[C@@H](CN2CCOC[C@H]2C)CN1 ; C[C@@H]1CN(CC(=O)N2CC(C)(C)c3c2cc(Cc2ccc(F)cc2)c2n[nH]c(=O)n32)[C@@H](CN2CCOC[C@H]2C)CN1] 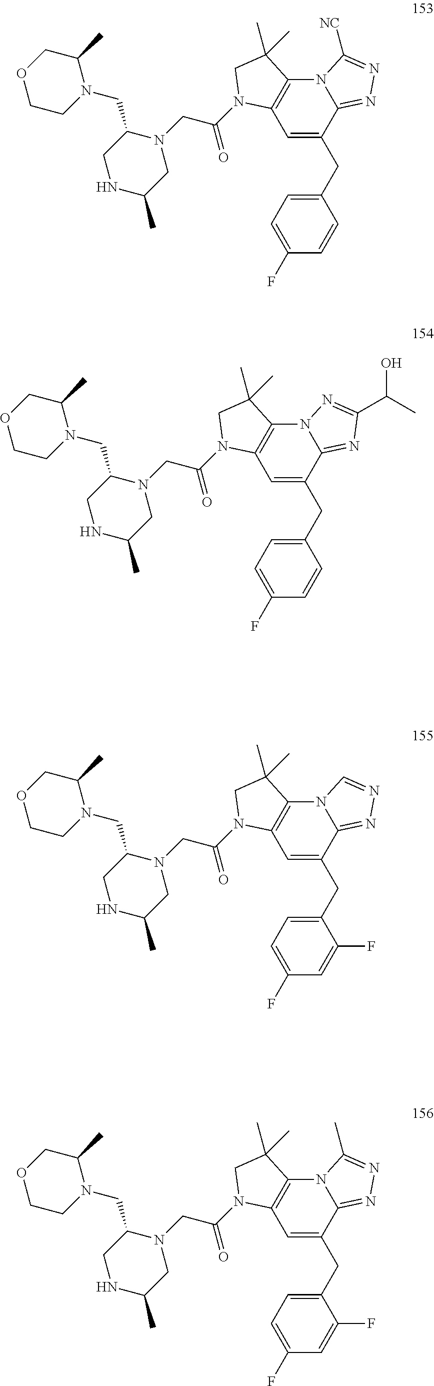 [SMILES: C[C@@H]1CN(CC(=O)N2CC(C)(C)c3c2cc(Cc2ccc(F)cc2)c2nnc(C#N)n32)[C@@H](CN2CCOC[C@H]2C)CN1 ; CC(O)c1nc2c(Cc3ccc(F)cc3)cc3c(n2n1)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2F)c2nncn32)[C@@H](CN2CCOC[C@H]2C)CN1 ; Cc1nnc2c(Cc3ccc(F)cc3F)cc3c(n12)C(C)(C)CN3C(=O)CN1C[C@@H](C)NC[C@@H]1CN1CCOC[C@H]1C] 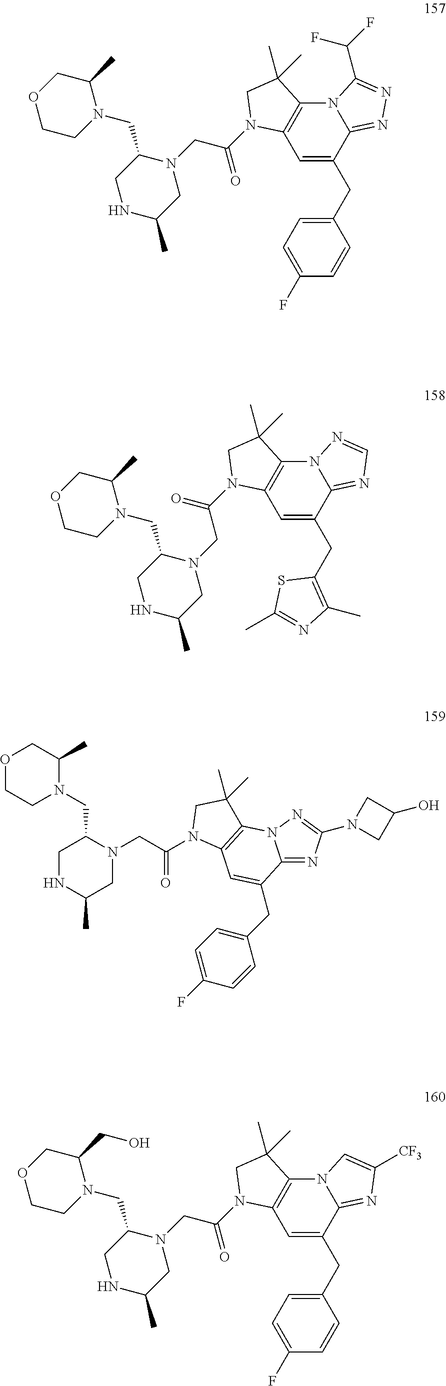 [SMILES: C[C@@H]1CN(CC(=O)N2CC(C)(C)c3c2cc(Cc2ccc(F)cc2)c2nnc(C(F)F)n32)[C@@H](CN2CCOC[C@H]2C)CN1 ; Cc1nc(C)c(Cc2cc3c(n4ncnc24)C(C)(C)CN3C(=O)CN2C[C@@H](C)NC[C@@H]2CN2CCOC[C@H]2C)s1 ; C[C@@H]1CN(CC(=O)N2CC(C)(C)c3c2cc(Cc2ccc(F)cc2)c2nc(N4CC(O)C4)nn32)[C@@H](CN2CCOC[C@H]2C)CN1 ; C[C@@H]1CN(CC(=O)N2CC(C)(C)c3c2cc(Cc2ccc(F)cc2)c2nc(C(F)(F)F)cn32)[C@@H](CN2CCOC[C@H]2CO)CN1] 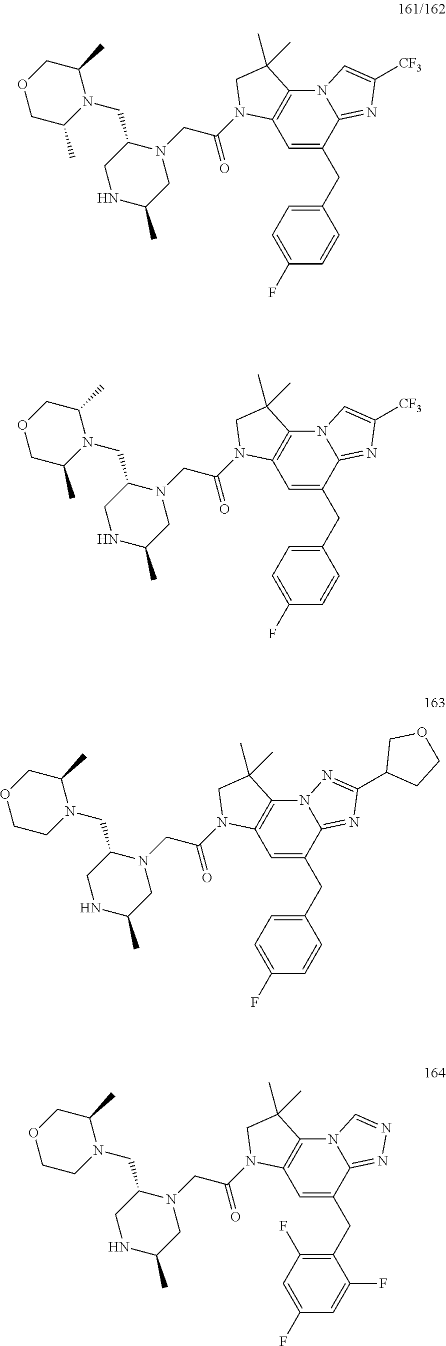 [SMILES: C[C@@H]1CN(CC(=O)N2CC(C)(C)c3c2cc(Cc2ccc(F)cc2)c2nc(C(F)(F)F)cn32)[C@@H](CN2[C@H](C)COC[C@H]2C)CN1 ; C[C@@H]1CN(CC(=O)N2CC(C)(C)c3c2cc(Cc2ccc(F)cc2)c2nc(C(F)(F)F)cn32)[C@@H](CN2[C@@H](C)COC[C@@H]2C)CN1 ; C[C@@H]1CN(CC(=O)N2CC(C)(C)c3c2cc(Cc2ccc(F)cc2)c2nc(C4CCOC4)nn32)[C@@H](CN2CCOC[C@H]2C)CN1 ; C[C@@H]1CN(CC(=O)N2CC(C)(C)c3c2cc(Cc2c(F)cc(F)cc2F)c2nncn32)[C@@H](CN2CCOC[C@H]2C)CN1] 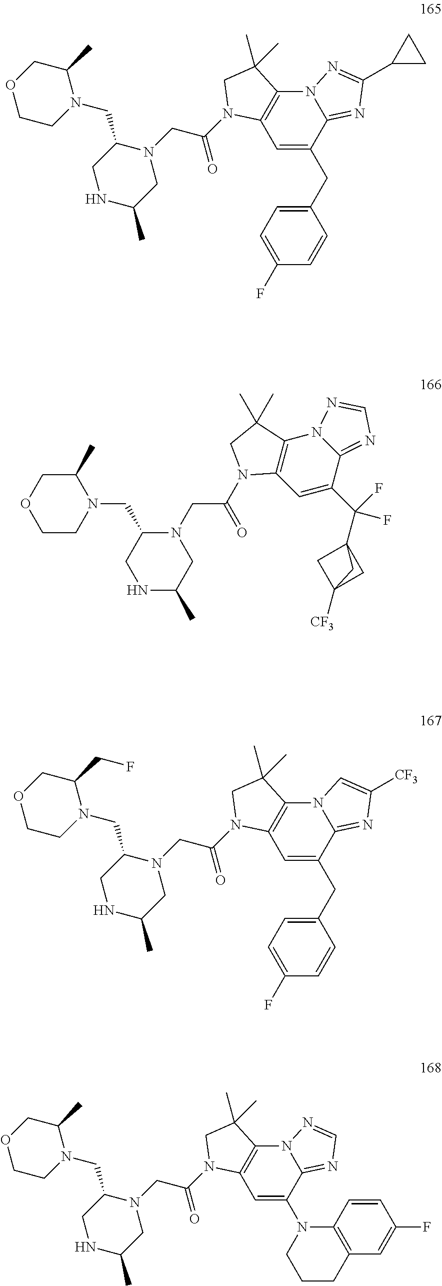 [SMILES: C[C@@H]1CN(CC(=O)N2CC(C)(C)c3c2cc(Cc2ccc(F)cc2)c2nc(C4CC4)nn32)[C@@H](CN2CCOC[C@H]2C)CN1 ; C[C@@H]1CN(CC(=O)N2CC(C)(C)c3c2cc(C(F)(F)C24CC(C(F)(F)F)(C2)C4)c2ncnn32)[C@@H](CN2CCOC[C@H]2C)CN1 ; C[C@@H]1CN(CC(=O)N2CC(C)(C)c3c2cc(Cc2ccc(F)cc2)c2nc(C(F)(F)F)cn32)[C@@H](CN2CCOC[C@H]2CF)CN1 ; C[C@@H]1CN(CC(=O)N2CC(C)(C)c3c2cc(N2CCCc4cc(F)ccc42)c2ncnn32)[C@@H](CN2CCOC[C@H]2C)CN1] 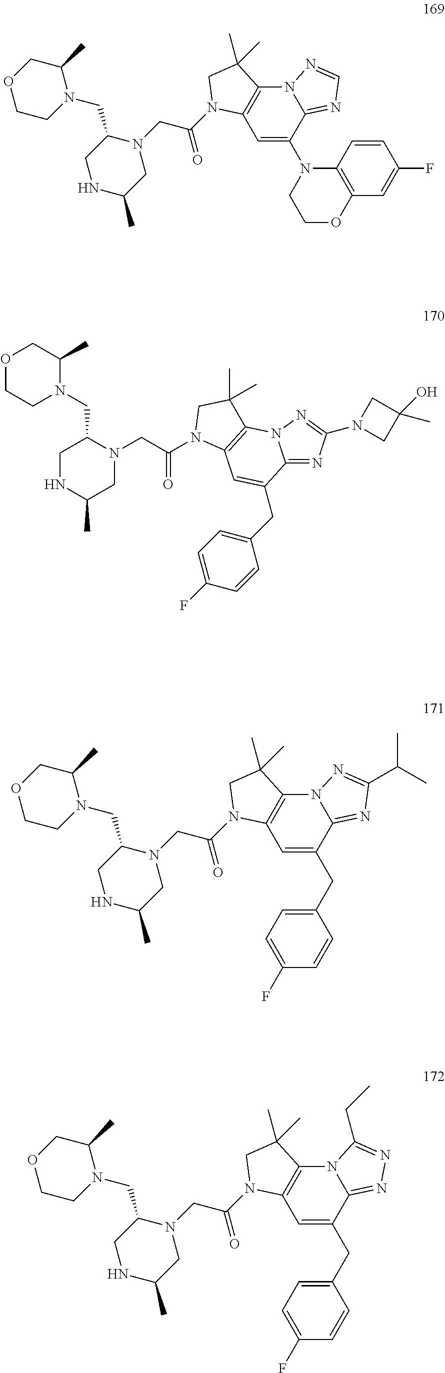 [SMILES: C[C@@H]1CN(CC(=O)N2CC(C)(C)c3c2cc(N2CCOc4cc(F)ccc42)c2ncnn32)[C@@H](CN2CCOC[C@H]2C)CN1 ; C[C@@H]1CN(CC(=O)N2CC(C)(C)c3c2cc(Cc2ccc(F)cc2)c2nc(N4CC(C)(O)C4)nn32)[C@@H](CN2CCOC[C@H]2C)CN1 ; CC(C)c1nc2c(Cc3ccc(F)cc3)cc3c(n2n1)C(C)(C)CN3C(=O)CN1C[C@@H](C)NC[C@@H]1CN1CCOC[C@H]1C ; CCc1nnc2c(Cc3ccc(F)cc3)cc3c(n12)C(C)(C)CN3C(=O)CN1C[C@@H](C)NC[C@@H]1CN1CCOC[C@H]1C] 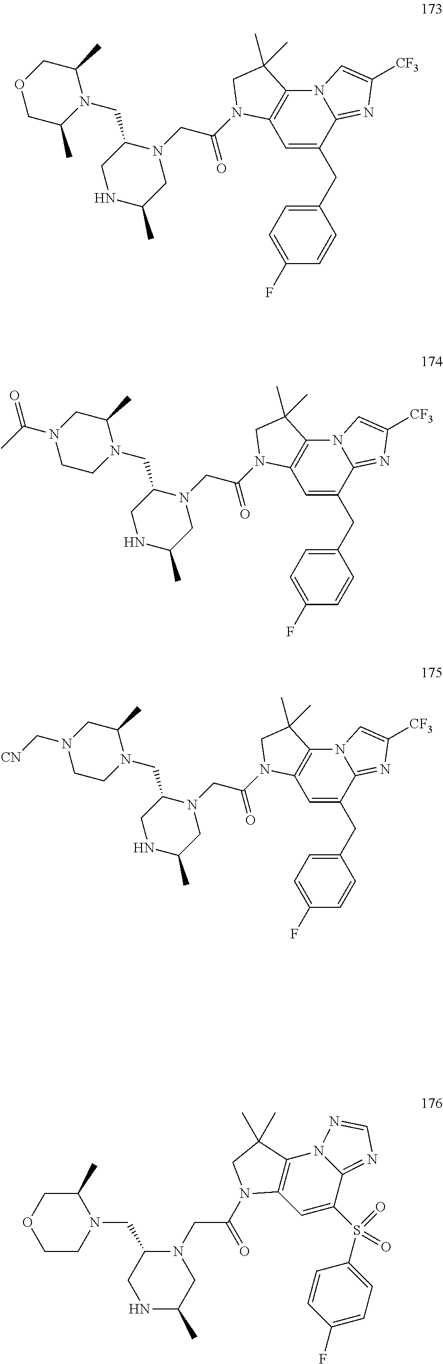 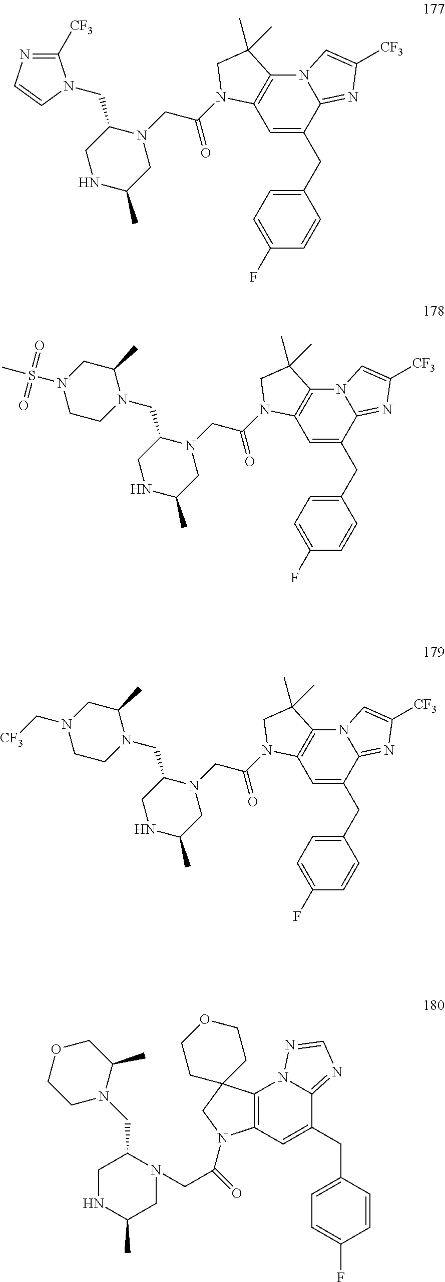 [SMILES: C[C@@H]1CN(CC(=O)N2CC(C)(C)c3c2cc(Cc2ccc(F)cc2)c2nc(C(F)(F)F)cn32)[C@@H](Cn2ccnc2C(F)(F)F)CN1 ; C[C@@H]1CN(CC(=O)N2CC(C)(C)c3c2cc(Cc2ccc(F)cc2)c2nc(C(F)(F)F)cn32)[C@@H](CN2CCN(S(C)(=O)=O)C[C@H]2C)CN1 ; C[C@@H]1CN(CC(=O)N2CC(C)(C)c3c2cc(Cc2ccc(F)cc2)c2nc(C(F)(F)F)cn32)[C@@H](CN2CCN(CC(F)(F)F)C[C@H]2C)CN1 ; C[C@@H]1CN(CC(=O)N2CC3(CCOCC3)c3c2cc(Cc2ccc(F)cc2)c2ncnn32)[C@@H](CN2CCOC[C@H]2C)CN1] 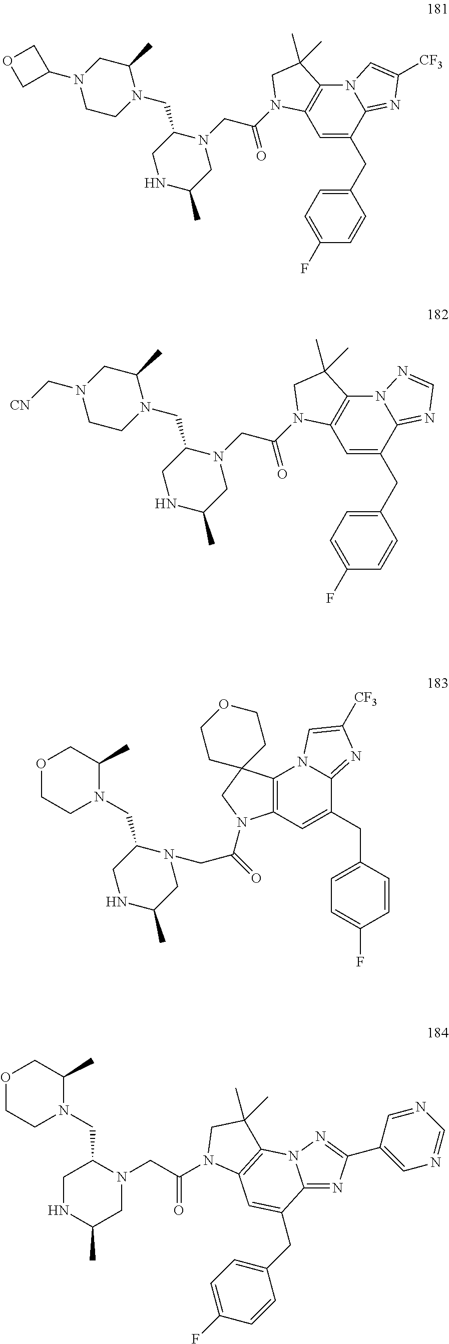 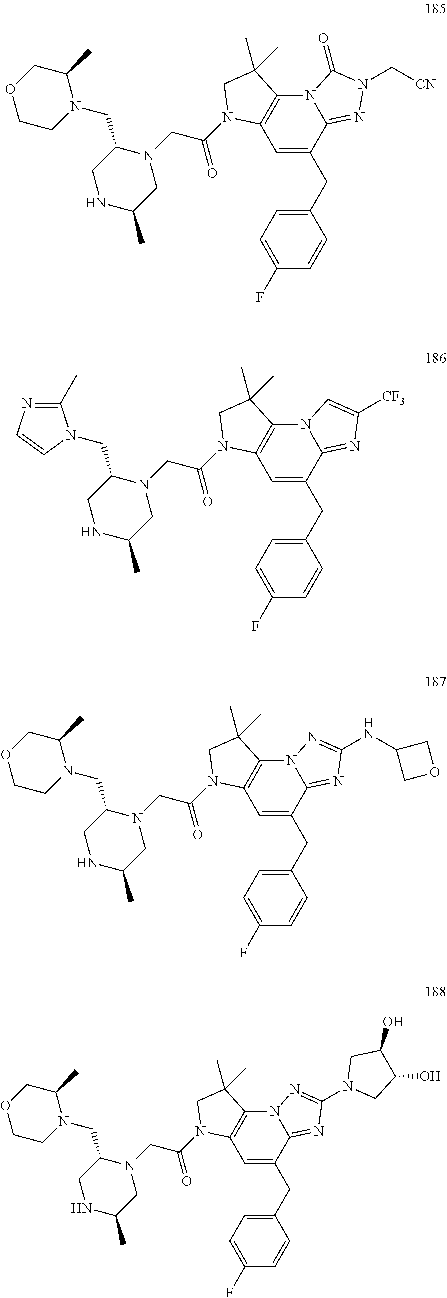 [SMILES: C[C@@H]1CN(CC(=O)N2CC(C)(C)c3c2cc(Cc2ccc(F)cc2)c2nn(CC#N)c(=O)n32)[C@@H](CN2CCOC[C@H]2C)CN1 ; Cc1nccn1C[C@H]1CN[C@H](C)CN1CC(=O)N1CC(C)(C)c2c1cc(Cc1ccc(F)cc1)c1nc(C(F)(F)F)cn21 ; C[C@@H]1CN(CC(=O)N2CC(C)(C)c3c2cc(Cc2ccc(F)cc2)c2nc(NC4COC4)nn32)[C@@H](CN2CCOC[C@H]2C)CN1 ; C[C@@H]1CN(CC(=O)N2CC(C)(C)c3c2cc(Cc2ccc(F)cc2)c2nc(N4C[C@@H](O)[C@H](O)C4)nn32)[C@@H](CN2CCOC[C@H]2C)CN1] 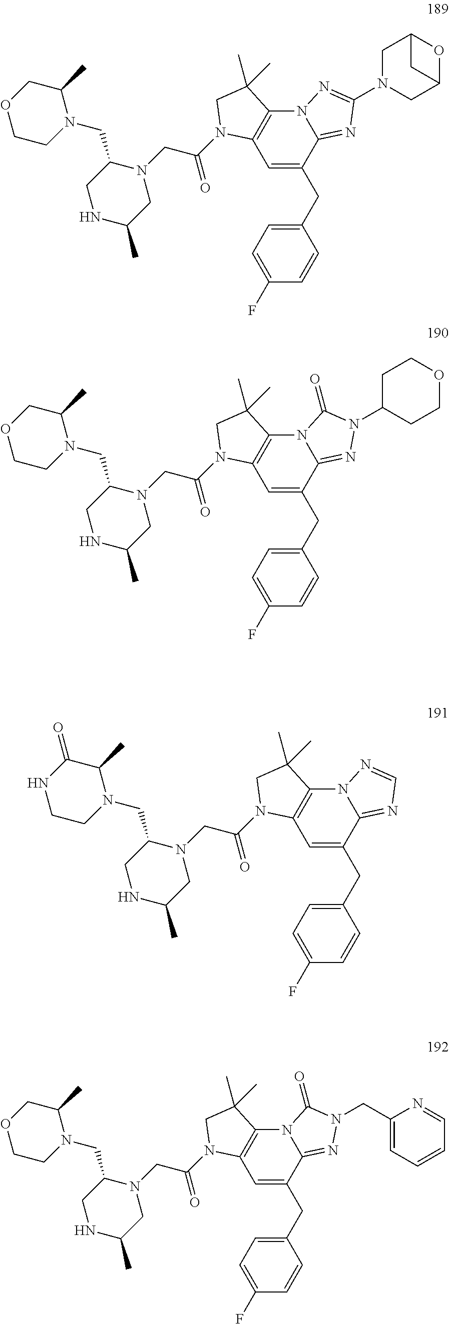 [SMILES: C[C@@H]1CN(CC(=O)N2CC(C)(C)c3c2cc(Cc2ccc(F)cc2)c2nc(N4CC5CC(C4)O5)nn32)[C@@H](CN2CCOC[C@H]2C)CN1 ; C[C@@H]1CN(CC(=O)N2CC(C)(C)c3c2cc(Cc2ccc(F)cc2)c2nn(C4CCOCC4)c(=O)n32)[C@@H](CN2CCOC[C@H]2C)CN1 ; C[C@@H]1CN(CC(=O)N2CC(C)(C)c3c2cc(Cc2ccc(F)cc2)c2ncnn32)[C@@H](CN2CCNC(=O)[C@H]2C)CN1 ; C[C@@H]1CN(CC(=O)N2CC(C)(C)c3c2cc(Cc2ccc(F)cc2)c2nn(Cc4ccccn4)c(=O)n32)[C@@H](CN2CCOC[C@H]2C)CN1] 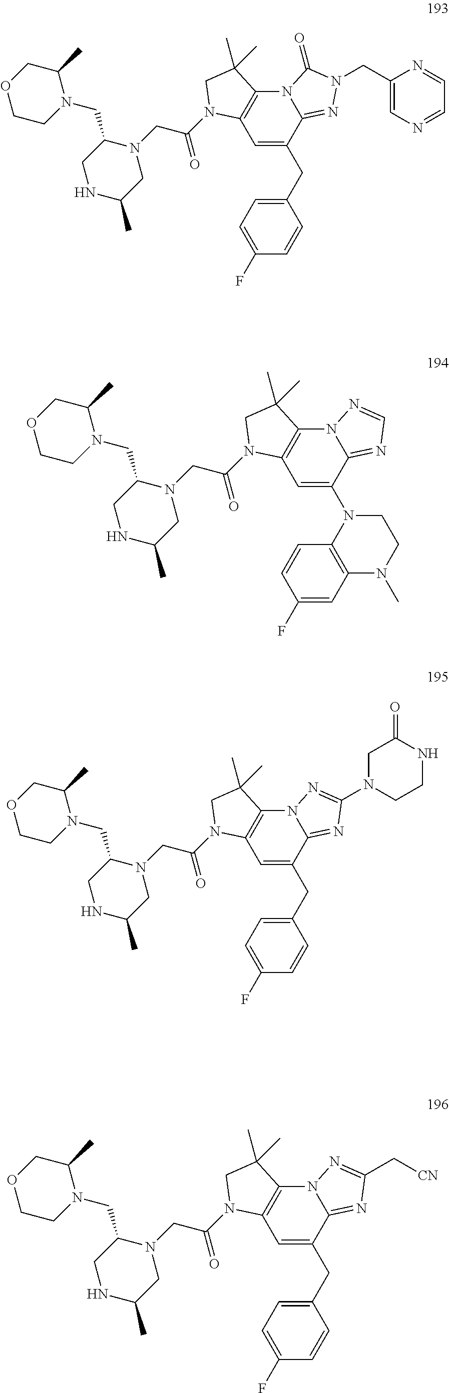 [SMILES: C[C@@H]1CN(CC(=O)N2CC(C)(C)c3c2cc(Cc2ccc(F)cc2)c2nn(Cc4cnccn4)c(=O)n32)[C@@H](CN2CCOC[C@H]2C)CN1 ; C[C@@H]1CN(CC(=O)N2CC(C)(C)c3c2cc(N2CCN(C)c4cc(F)ccc42)c2ncnn32)[C@@H](CN2CCOC[C@H]2C)CN1 ; C[C@@H]1CN(CC(=O)N2CC(C)(C)c3c2cc(Cc2ccc(F)cc2)c2nc(N4CCNC(=O)C4)nn32)[C@@H](CN2CCOC[C@H]2C)CN1 ; C[C@@H]1CN(CC(=O)N2CC(C)(C)c3c2cc(Cc2ccc(F)cc2)c2nc(CC#N)nn32)[C@@H](CN2CCOC[C@H]2C)CN1] 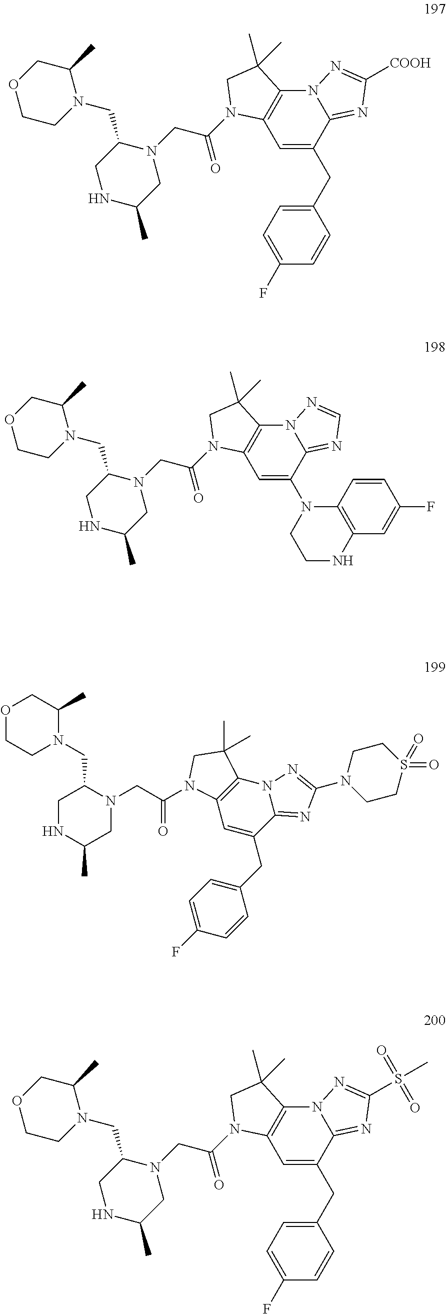 [SMILES: C[C@@H]1CN(CC(=O)N2CC(C)(C)c3c2cc(Cc2ccc(F)cc2)c2nc(C(=O)O)nn32)[C@@H](CN2CCOC[C@H]2C)CN1 ; C[C@@H]1CN(CC(=O)N2CC(C)(C)c3c2cc(N2CCNc4cc(F)ccc42)c2ncnn32)[C@@H](CN2CCOC[C@H]2C)CN1 ; C[C@@H]1CN(CC(=O)N2CC(C)(C)c3c2cc(Cc2ccc(F)cc2)c2nc(N4CCS(=O)(=O)CC4)nn32)[C@@H](CN2CCOC[C@H]2C)CN1 ; C[C@@H]1CN(CC(=O)N2CC(C)(C)c3c2cc(Cc2ccc(F)cc2)c2nc(S(C)(=O)=O)nn32)[C@@H](CN2CCOC[C@H]2C)CN1] 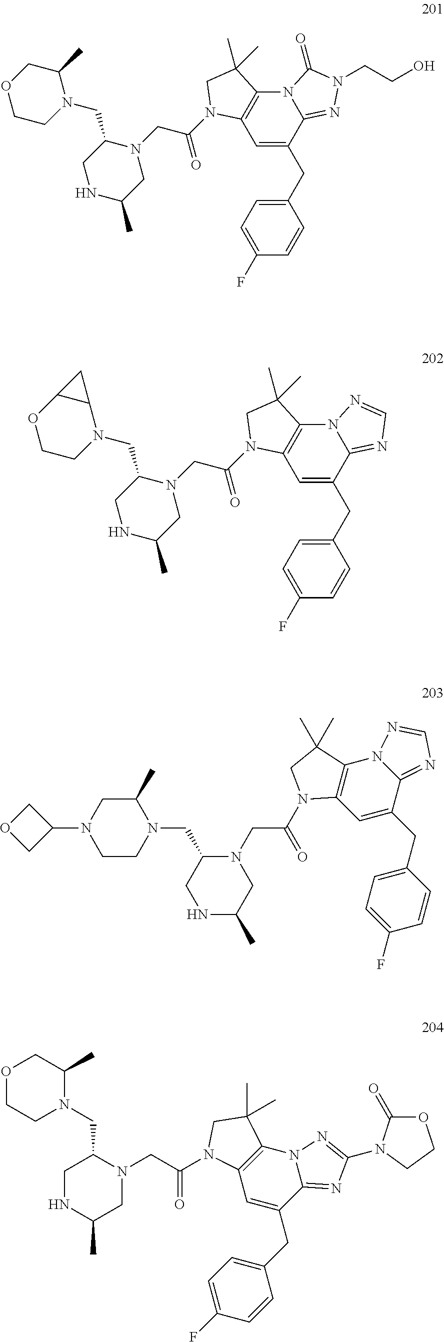 [SMILES: C[C@@H]1CN(CC(=O)N2CC(C)(C)c3c2cc(Cc2ccc(F)cc2)c2nn(CCO)c(=O)n32)[C@@H](CN2CCOC[C@H]2C)CN1 ; C[C@@H]1CN(CC(=O)N2CC(C)(C)c3c2cc(Cc2ccc(F)cc2)c2ncnn32)[C@@H](CN2CCOC3CC32)CN1 ; C[C@@H]1CN(CC(=O)N2CC(C)(C)c3c2cc(Cc2ccc(F)cc2)c2ncnn32)[C@@H](CN2CCN(C3COC3)C[C@H]2C)CN1 ; C[C@@H]1CN(CC(=O)N2CC(C)(C)c3c2cc(Cc2ccc(F)cc2)c2nc(N4CCOC4=O)nn32)[C@@H](CN2CCOC[C@H]2C)CN1] 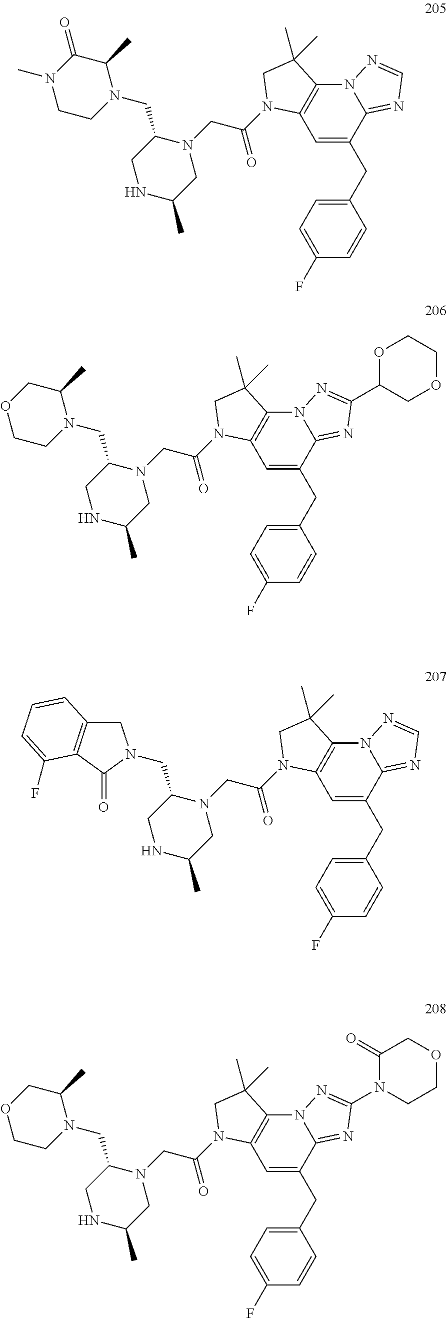 [SMILES: C[C@@H]1CN(CC(=O)N2CC(C)(C)c3c2cc(Cc2ccc(F)cc2)c2ncnn32)[C@@H](CN2CCN(C)C(=O)[C@H]2C)CN1 ; C[C@@H]1CN(CC(=O)N2CC(C)(C)c3c2cc(Cc2ccc(F)cc2)c2nc(C4COCCO4)nn32)[C@@H](CN2CCOC[C@H]2C)CN1 ; C[C@@H]1CN(CC(=O)N2CC(C)(C)c3c2cc(Cc2ccc(F)cc2)c2ncnn32)[C@@H](CN2Cc3cccc(F)c3C2=O)CN1 ; C[C@@H]1CN(CC(=O)N2CC(C)(C)c3c2cc(Cc2ccc(F)cc2)c2nc(N4CCOCC4=O)nn32)[C@@H](CN2CCOC[C@H]2C)CN1] 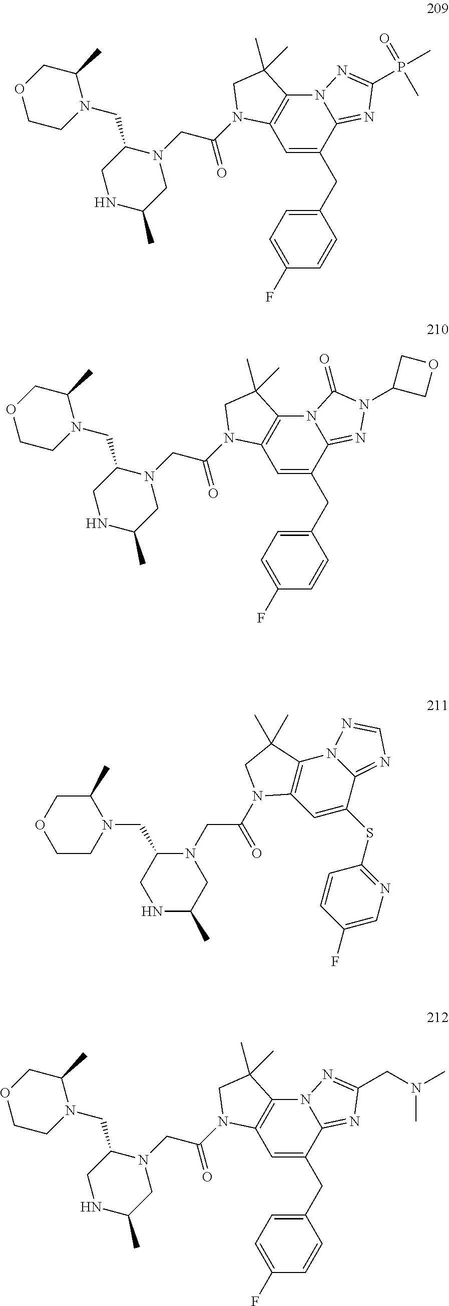 [SMILES: C[C@@H]1CN(CC(=O)N2CC(C)(C)c3c2cc(Cc2ccc(F)cc2)c2nc(P(C)(C)=O)nn32)[C@@H](CN2CCOC[C@H]2C)CN1 ; C[C@@H]1CN(CC(=O)N2CC(C)(C)c3c2cc(Cc2ccc(F)cc2)c2nn(C4COC4)c(=O)n32)[C@@H](CN2CCOC[C@H]2C)CN1 ; C[C@@H]1CN(CC(=O)N2CC(C)(C)c3c2cc(Sc2ccc(F)cn2)c2ncnn32)[C@@H](CN2CCOC[C@H]2C)CN1 ; C[C@@H]1CN(CC(=O)N2CC(C)(C)c3c2cc(Cc2ccc(F)cc2)c2nc(CN(C)C)nn32)[C@@H](CN2CCOC[C@H]2C)CN1] 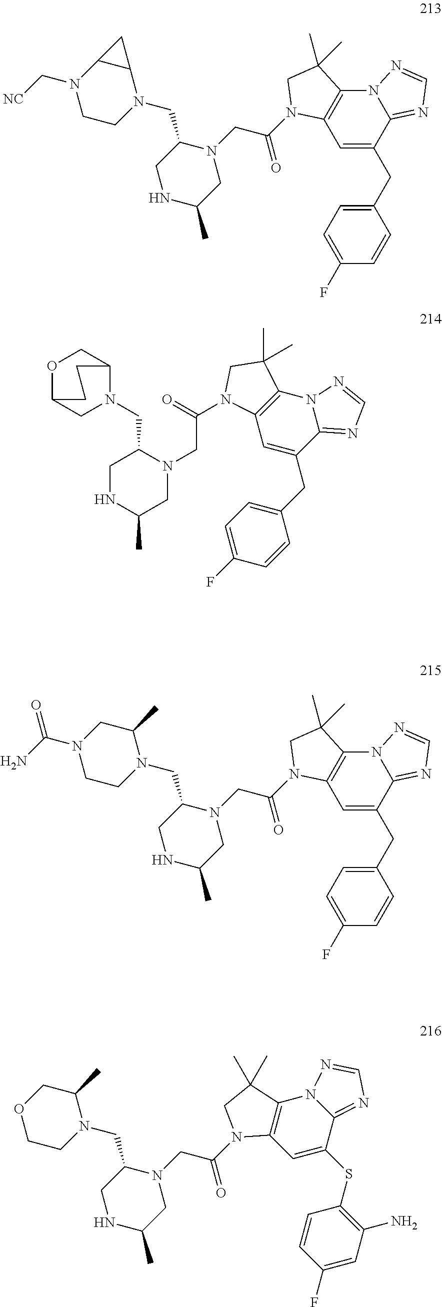 [SMILES: C[C@@H]1CN(CC(=O)N2CC(C)(C)c3c2cc(Cc2ccc(F)cc2)c2ncnn32)[C@@H](CN2CCN(CC#N)C3CC32)CN1 ; C[C@@H]1CN(CC(=O)N2CC(C)(C)c3c2cc(Cc2ccc(F)cc2)c2ncnn32)[C@@H](CN2CC3CCC2CO3)CN1 ; C[C@@H]1CN(CC(=O)N2CC(C)(C)c3c2cc(Cc2ccc(F)cc2)c2ncnn32)[C@@H](CN2CCN(C(N)=O)C[C@H]2C)CN1 ; C[C@@H]1CN(CC(=O)N2CC(C)(C)c3c2cc(Sc2ccc(F)cc2N)c2ncnn32)[C@@H](CN2CCOC[C@H]2C)CN1] 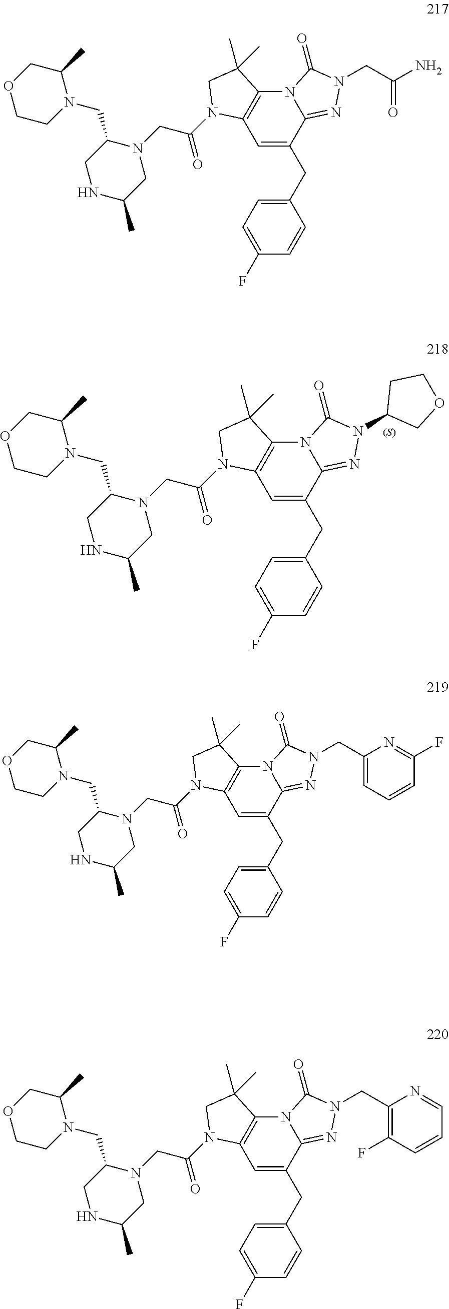 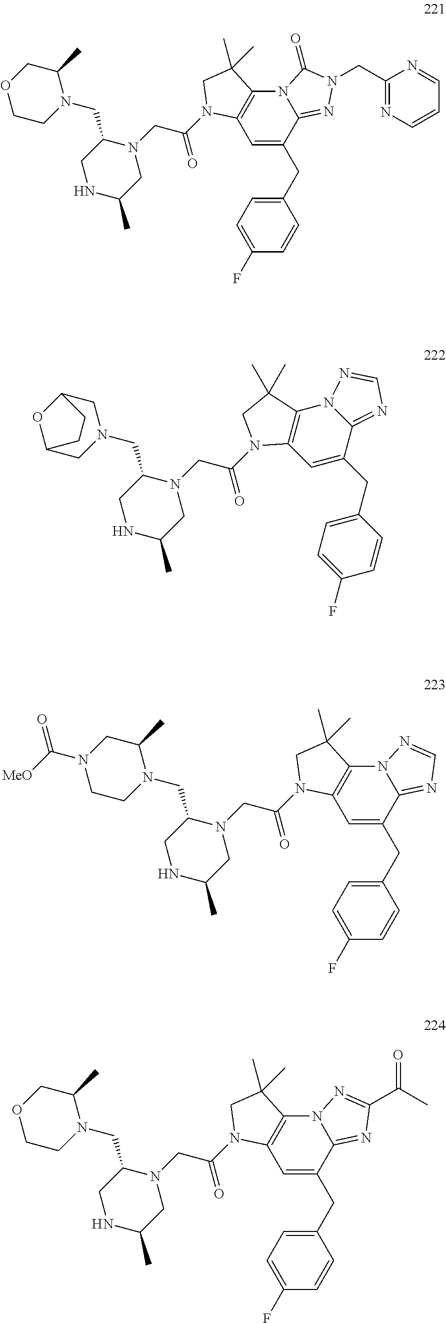 [SMILES: C[C@@H]1CN(CC(=O)N2CC(C)(C)c3c2cc(Cc2ccc(F)cc2)c2nn(Cc4ncccn4)c(=O)n32)[C@@H](CN2CCOC[C@H]2C)CN1 ; C[C@@H]1CN(CC(=O)N2CC(C)(C)c3c2cc(Cc2ccc(F)cc2)c2ncnn32)[C@@H](CN2CC3CCC(C2)O3)CN1 ; COC(=O)N1CCN(C[C@H]2CN[C@H](C)CN2CC(=O)N2CC(C)(C)c3c2cc(Cc2ccc(F)cc2)c2ncnn32)[C@H](C)C1 ; CC(=O)c1nc2c(Cc3ccc(F)cc3)cc3c(n2n1)C(C)(C)CN3C(=O)CN1C[C@@H](C)NC[C@@H]1CN1CCOC[C@H]1C] 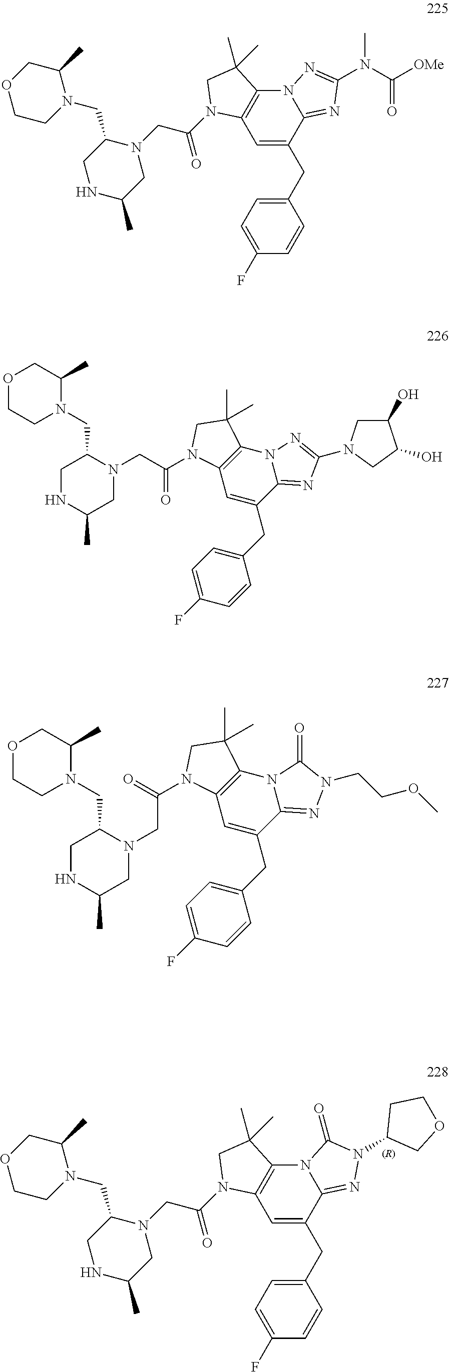 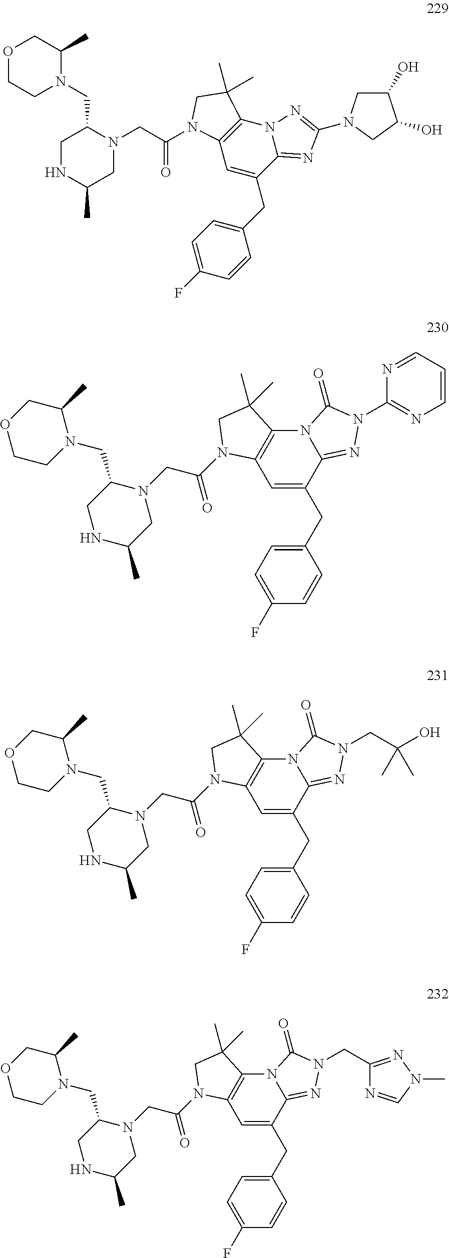 [SMILES: C[C@@H]1CN(CC(=O)N2CC(C)(C)c3c2cc(Cc2ccc(F)cc2)c2nc(N4C[C@@H](O)[C@@H](O)C4)nn32)[C@@H](CN2CCOC[C@H]2C)CN1 ; C[C@@H]1CN(CC(=O)N2CC(C)(C)c3c2cc(Cc2ccc(F)cc2)c2nn(-c4ncccn4)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CC(C)(C)O)c(=O)n32)[C@@H](CN2CCOC[C@H]2C)CN1 ; C[C@@H]1CN(CC(=O)N2CC(C)(C)c3c2cc(Cc2ccc(F)cc2)c2nn(Cc4ncn(C)n4)c(=O)n32)[C@@H](CN2CCOC[C@H]2C)CN1] 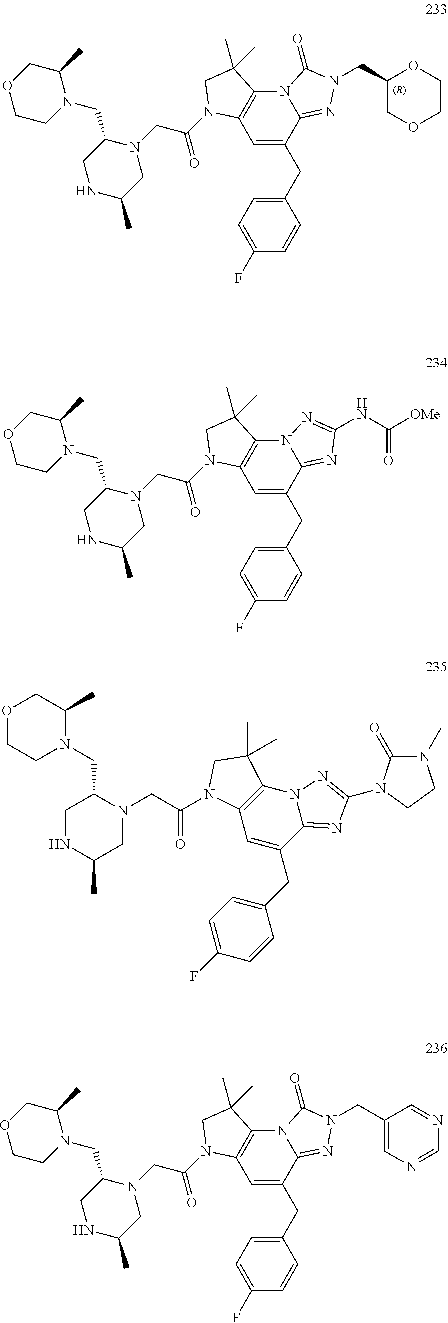 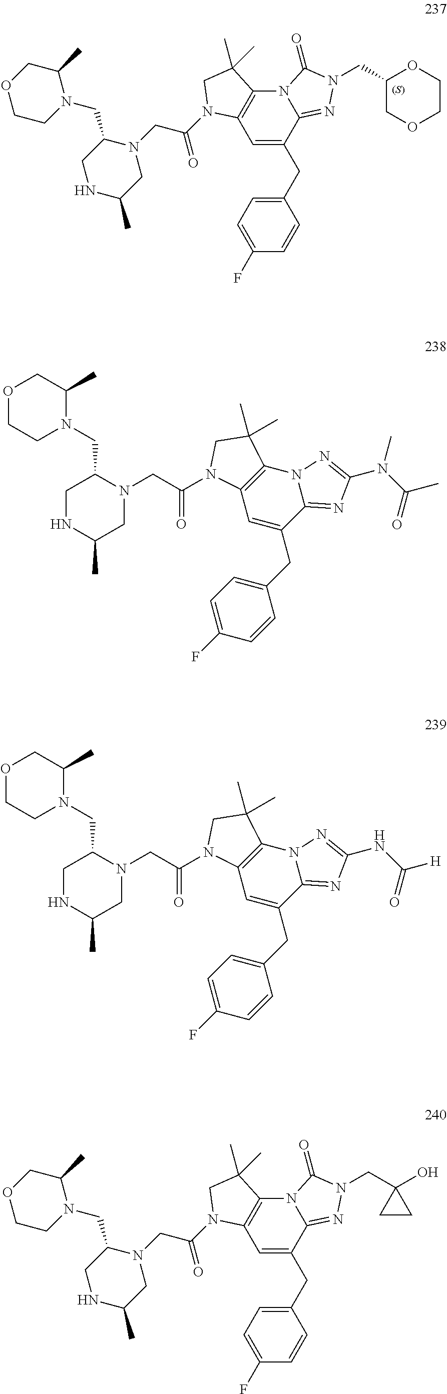 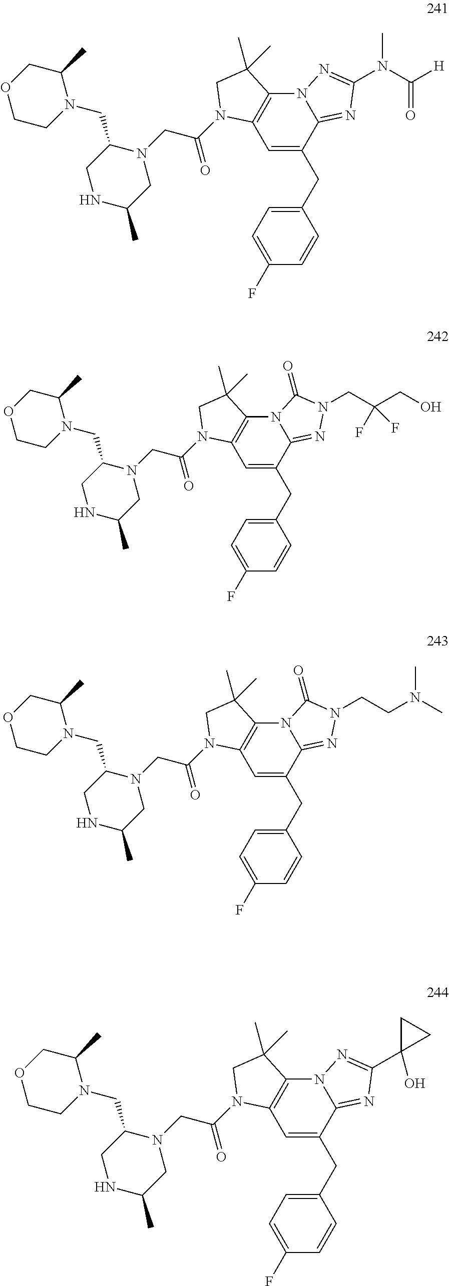 [SMILES: C[C@@H]1CN(CC(=O)N2CC(C)(C)c3c2cc(Cc2ccc(F)cc2)c2nc(N(C)C=O)nn32)[C@@H](CN2CCOC[C@H]2C)CN1 ; C[C@@H]1CN(CC(=O)N2CC(C)(C)c3c2cc(Cc2ccc(F)cc2)c2nn(CC(F)(F)CO)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CCN(C)C)c(=O)n32)[C@@H](CN2CCOC[C@H]2C)CN1 ; C[C@@H]1CN(CC(=O)N2CC(C)(C)c3c2cc(Cc2ccc(F)cc2)c2nc(C4(O)CC4)nn32)[C@@H](CN2CCOC[C@H]2C)CN1] 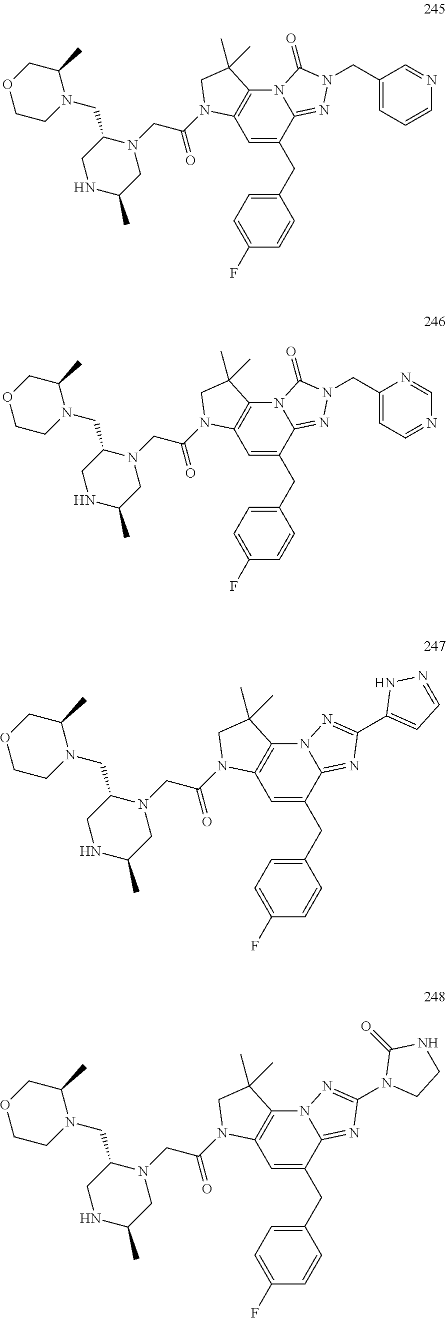 [SMILES: C[C@@H]1CN(CC(=O)N2CC(C)(C)c3c2cc(Cc2ccc(F)cc2)c2nn(Cc4cccnc4)c(=O)n32)[C@@H](CN2CCOC[C@H]2C)CN1 ; C[C@@H]1CN(CC(=O)N2CC(C)(C)c3c2cc(Cc2ccc(F)cc2)c2nn(Cc4ccncn4)c(=O)n32)[C@@H](CN2CCOC[C@H]2C)CN1 ; C[C@@H]1CN(CC(=O)N2CC(C)(C)c3c2cc(Cc2ccc(F)cc2)c2nc(-c4ccn[nH]4)nn32)[C@@H](CN2CCOC[C@H]2C)CN1 ; C[C@@H]1CN(CC(=O)N2CC(C)(C)c3c2cc(Cc2ccc(F)cc2)c2nc(N4CCNC4=O)nn32)[C@@H](CN2CCOC[C@H]2C)CN1] 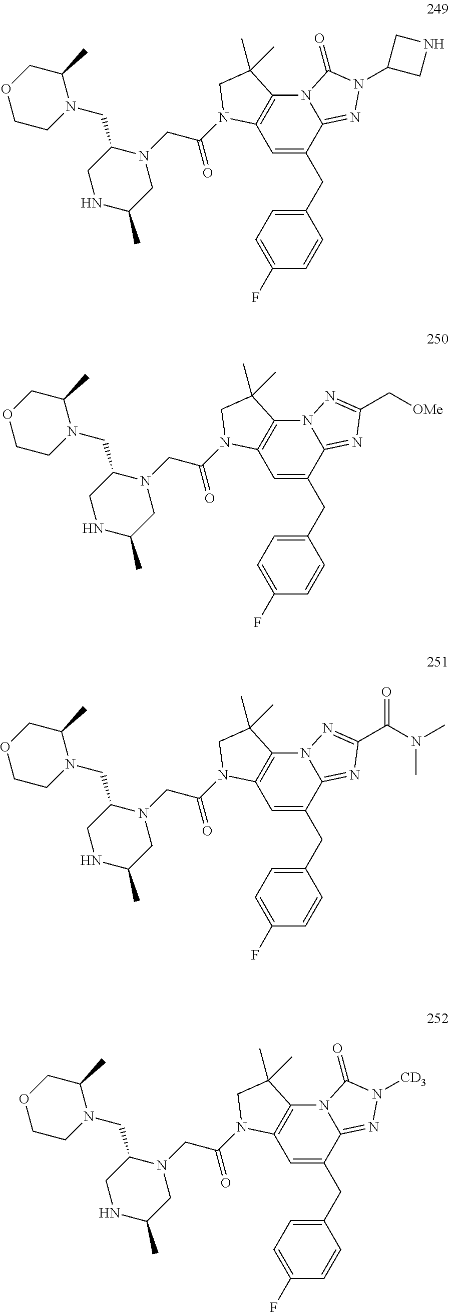 [SMILES: C[C@@H]1CN(CC(=O)N2CC(C)(C)c3c2cc(Cc2ccc(F)cc2)c2nn(C4CNC4)c(=O)n32)[C@@H](CN2CCOC[C@H]2C)CN1 ; COCc1nc2c(Cc3ccc(F)cc3)cc3c(n2n1)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2nc(C(=O)N(C)C)nn32)[C@@H](CN2CCOC[C@H]2C)CN1 ; [2H]C([2H])([2H])n1nc2c(Cc3ccc(F)cc3)cc3c(n2c1=O)C(C)(C)CN3C(=O)CN1C[C@@H](C)NC[C@@H]1CN1CCOC[C@H]1C] 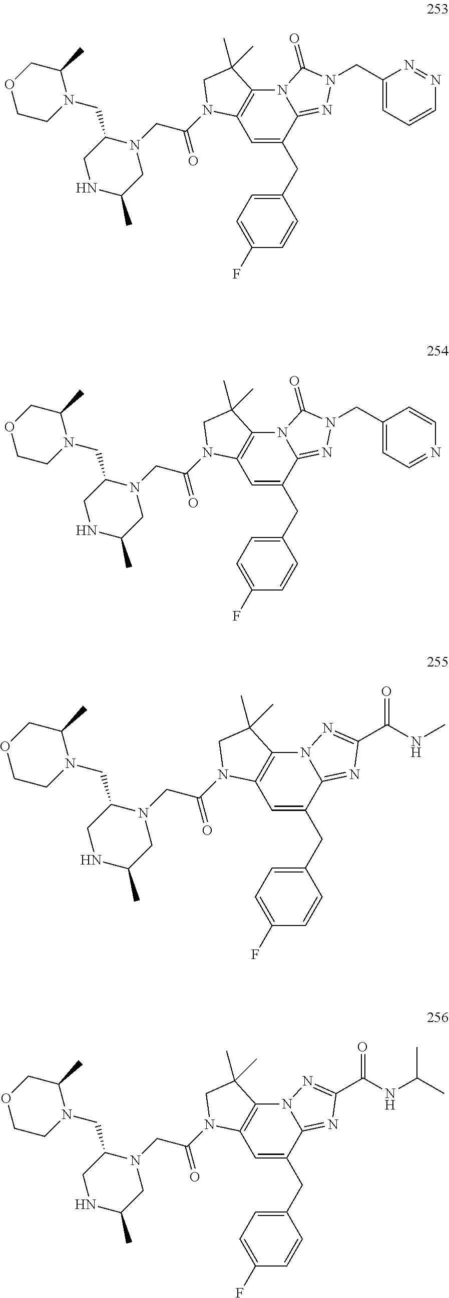 [SMILES: C[C@@H]1CN(CC(=O)N2CC(C)(C)c3c2cc(Cc2ccc(F)cc2)c2nn(Cc4cccnn4)c(=O)n32)[C@@H](CN2CCOC[C@H]2C)CN1 ; C[C@@H]1CN(CC(=O)N2CC(C)(C)c3c2cc(Cc2ccc(F)cc2)c2nn(Cc4ccncc4)c(=O)n32)[C@@H](CN2CCOC[C@H]2C)CN1 ; CNC(=O)c1nc2c(Cc3ccc(F)cc3)cc3c(n2n1)C(C)(C)CN3C(=O)CN1C[C@@H](C)NC[C@@H]1CN1CCOC[C@H]1C ; CC(C)NC(=O)c1nc2c(Cc3ccc(F)cc3)cc3c(n2n1)C(C)(C)CN3C(=O)CN1C[C@@H](C)NC[C@@H]1CN1CCOC[C@H]1C] 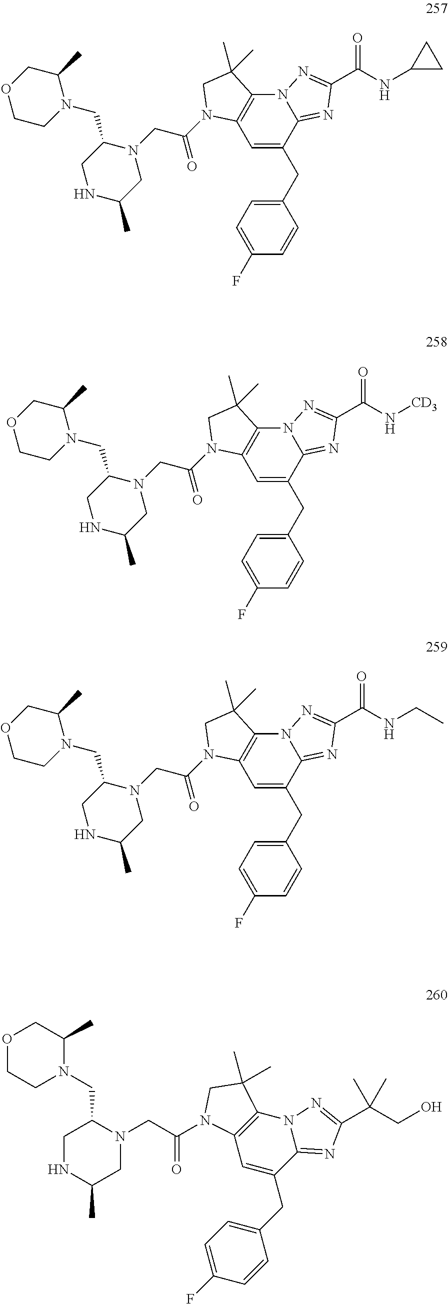 [SMILES: C[C@@H]1CN(CC(=O)N2CC(C)(C)c3c2cc(Cc2ccc(F)cc2)c2nc(C(=O)NC4CC4)nn32)[C@@H](CN2CCOC[C@H]2C)CN1 ; [2H]C([2H])([2H])NC(=O)c1nc2c(Cc3ccc(F)cc3)cc3c(n2n1)C(C)(C)CN3C(=O)CN1C[C@@H](C)NC[C@@H]1CN1CCOC[C@H]1C ; CCNC(=O)c1nc2c(Cc3ccc(F)cc3)cc3c(n2n1)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2nc(C(C)(C)CO)nn32)[C@@H](CN2CCOC[C@H]2C)CN1] 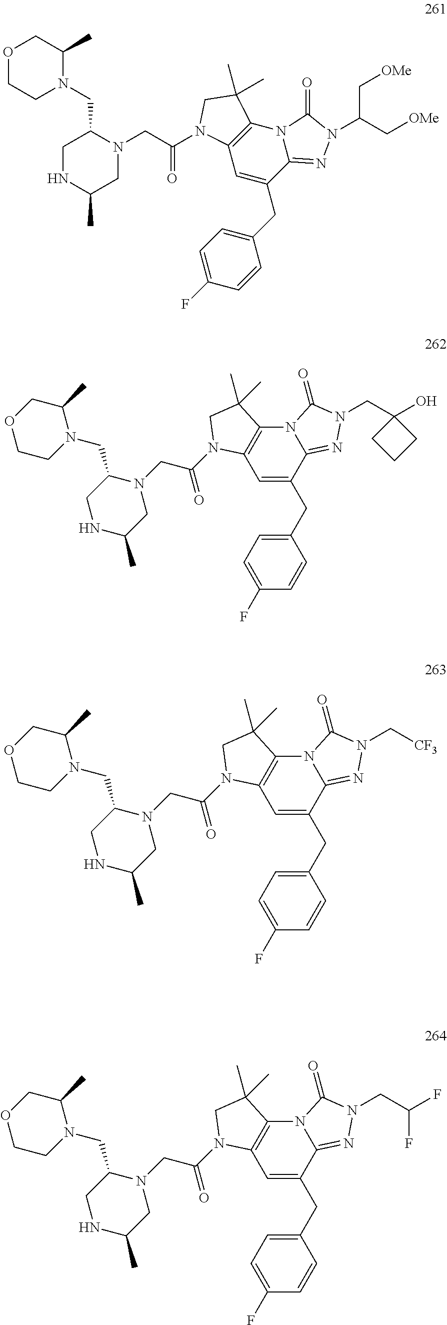 [SMILES: COCC(COC)n1nc2c(Cc3ccc(F)cc3)cc3c(n2c1=O)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2nn(CC4(O)CCC4)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CC(F)(F)F)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CC(F)F)c(=O)n32)[C@@H](CN2CCOC[C@H]2C)CN1] 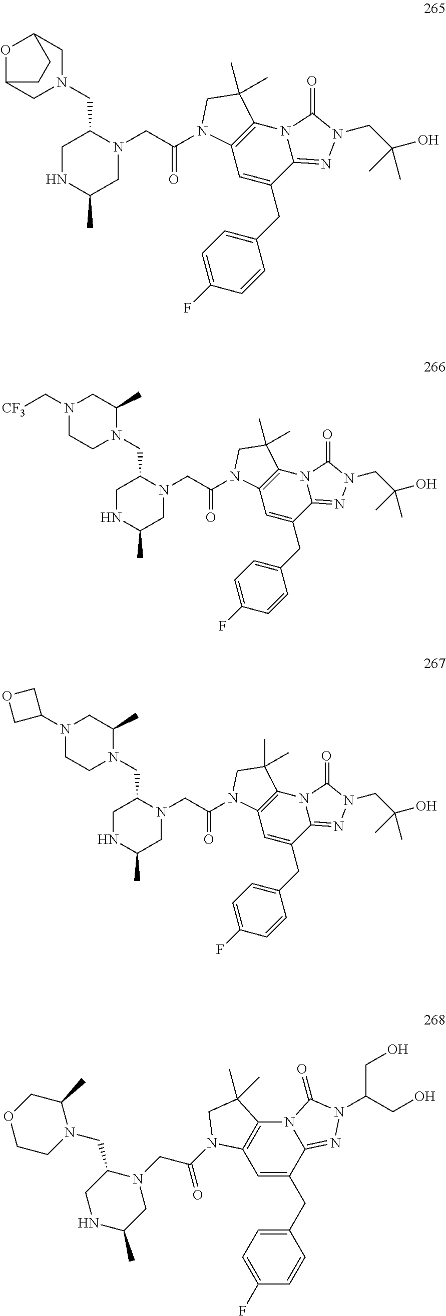 [SMILES: C[C@@H]1CN(CC(=O)N2CC(C)(C)c3c2cc(Cc2ccc(F)cc2)c2nn(CC(C)(C)O)c(=O)n32)[C@@H](CN2CC3CCC(C2)O3)CN1 ; C[C@@H]1CN(CC(=O)N2CC(C)(C)c3c2cc(Cc2ccc(F)cc2)c2nn(CC(C)(C)O)c(=O)n32)[C@@H](CN2CCN(CC(F)(F)F)C[C@H]2C)CN1 ; C[C@@H]1CN(CC(=O)N2CC(C)(C)c3c2cc(Cc2ccc(F)cc2)c2nn(CC(C)(C)O)c(=O)n32)[C@@H](CN2CCN(C3COC3)C[C@H]2C)CN1 ; C[C@@H]1CN(CC(=O)N2CC(C)(C)c3c2cc(Cc2ccc(F)cc2)c2nn(C(CO)CO)c(=O)n32)[C@@H](CN2CCOC[C@H]2C)CN1] 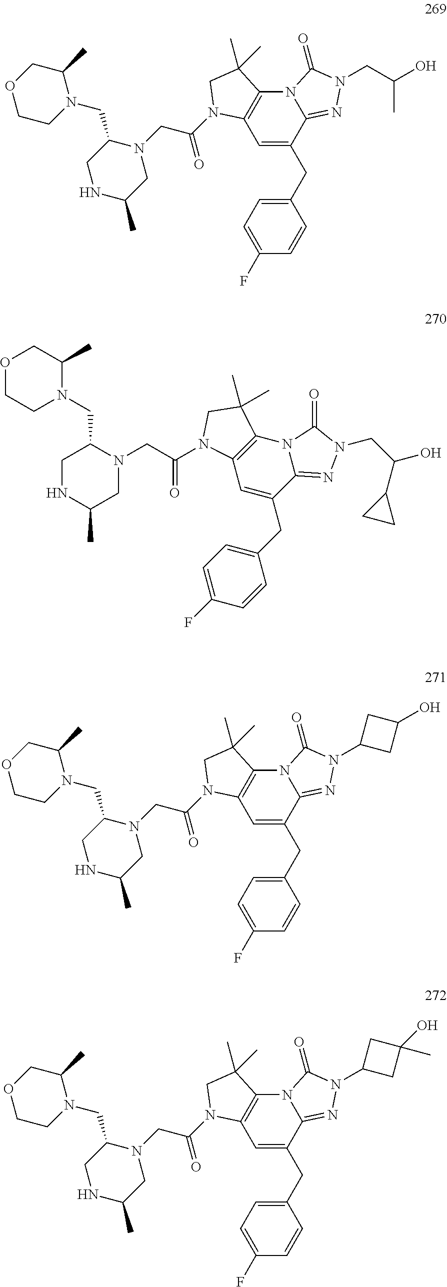 [SMILES: CC(O)Cn1nc2c(Cc3ccc(F)cc3)cc3c(n2c1=O)C(C)(C)CN3C(=O)CN1C[C@@H](C)NC[C@@H]1CN1CCOC[C@H]1C ; C[C@@H]1CN(CC(=O)N2CC(C)(C)c3c2cc(Cc2ccc(F)cc2)c2nn(CC(O)C4CC4)c(=O)n32)[C@@H](CN2CCOC[C@H]2C)CN1 ; C[C@@H]1CN(CC(=O)N2CC(C)(C)c3c2cc(Cc2ccc(F)cc2)c2nn(C4CC(O)C4)c(=O)n32)[C@@H](CN2CCOC[C@H]2C)CN1 ; C[C@@H]1CN(CC(=O)N2CC(C)(C)c3c2cc(Cc2ccc(F)cc2)c2nn(C4CC(C)(O)C4)c(=O)n32)[C@@H](CN2CCOC[C@H]2C)CN1] 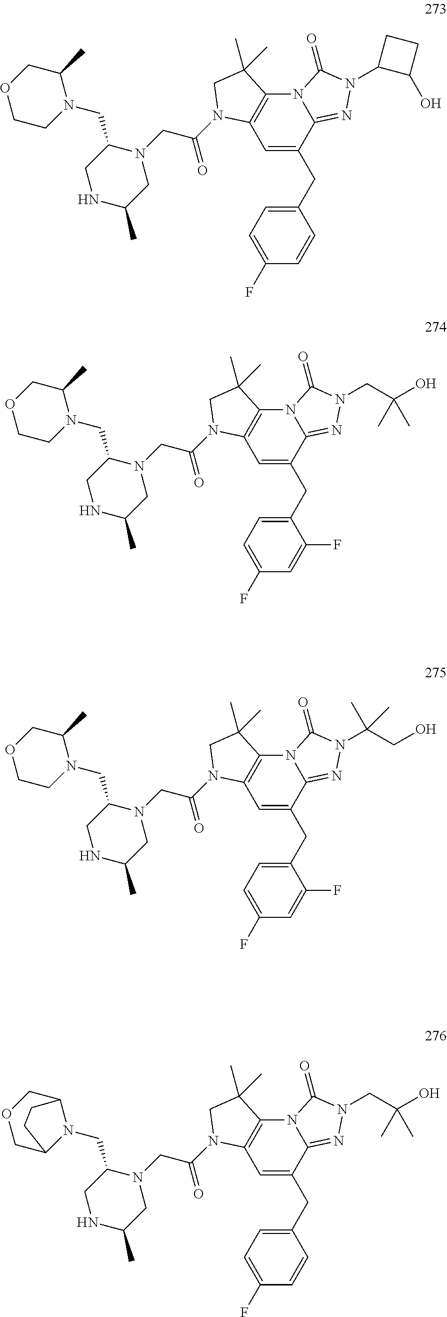 [SMILES: C[C@@H]1CN(CC(=O)N2CC(C)(C)c3c2cc(Cc2ccc(F)cc2)c2nn(C4CCC4O)c(=O)n32)[C@@H](CN2CCOC[C@H]2C)CN1 ; C[C@@H]1CN(CC(=O)N2CC(C)(C)c3c2cc(Cc2ccc(F)cc2F)c2nn(CC(C)(C)O)c(=O)n32)[C@@H](CN2CCOC[C@H]2C)CN1 ; C[C@@H]1CN(CC(=O)N2CC(C)(C)c3c2cc(Cc2ccc(F)cc2F)c2nn(C(C)(C)CO)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CC(C)(C)O)c(=O)n32)[C@@H](CN2C3CCC2COC3)CN1] 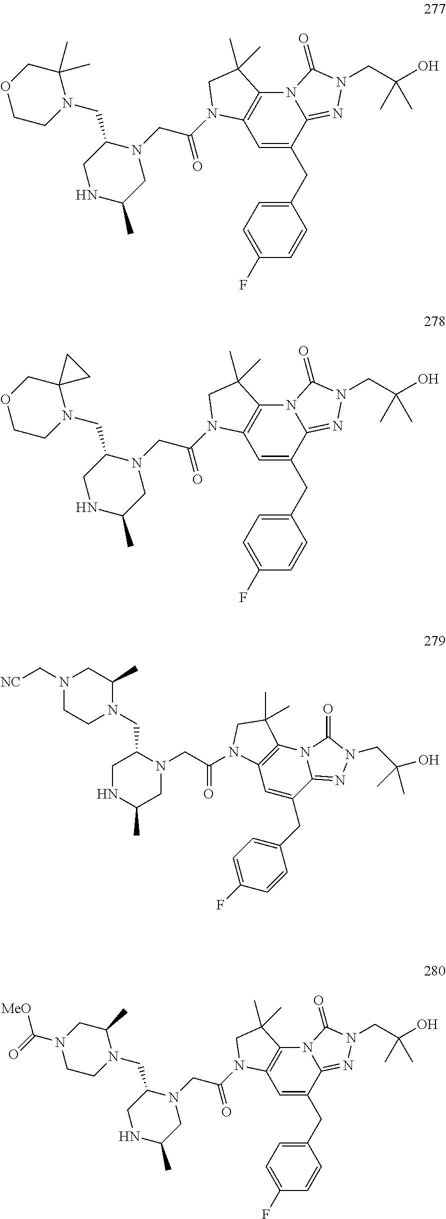 [SMILES: C[C@@H]1CN(CC(=O)N2CC(C)(C)c3c2cc(Cc2ccc(F)cc2)c2nn(CC(C)(C)O)c(=O)n32)[C@@H](CN2CCOCC2(C)C)CN1 ; C[C@@H]1CN(CC(=O)N2CC(C)(C)c3c2cc(Cc2ccc(F)cc2)c2nn(CC(C)(C)O)c(=O)n32)[C@@H](CN2CCOCC23CC3)CN1 ; C[C@@H]1CN(CC(=O)N2CC(C)(C)c3c2cc(Cc2ccc(F)cc2)c2nn(CC(C)(C)O)c(=O)n32)[C@@H](CN2CCN(CC#N)C[C@H]2C)CN1 ; COC(=O)N1CCN(C[C@H]2CN[C@H](C)CN2CC(=O)N2CC(C)(C)c3c2cc(Cc2ccc(F)cc2)c2nn(CC(C)(C)O)c(=O)n32)[C@H](C)C1] 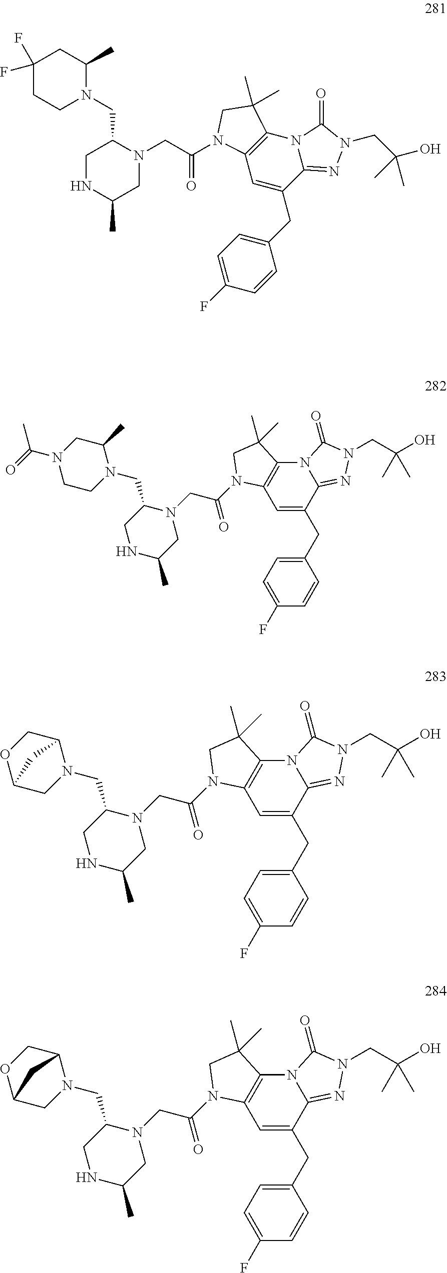 [SMILES: C[C@@H]1CN(CC(=O)N2CC(C)(C)c3c2cc(Cc2ccc(F)cc2)c2nn(CC(C)(C)O)c(=O)n32)[C@@H](CN2CCC(F)(F)C[C@H]2C)CN1 ; CC(=O)N1CCN(C[C@H]2CN[C@H](C)CN2CC(=O)N2CC(C)(C)c3c2cc(Cc2ccc(F)cc2)c2nn(CC(C)(C)O)c(=O)n32)[C@H](C)C1 ; C[C@@H]1CN(CC(=O)N2CC(C)(C)c3c2cc(Cc2ccc(F)cc2)c2nn(CC(C)(C)O)c(=O)n32)[C@@H](CN2C[C@@H]3C[C@H]2CO3)CN1 ; C[C@@H]1CN(CC(=O)N2CC(C)(C)c3c2cc(Cc2ccc(F)cc2)c2nn(CC(C)(C)O)c(=O)n32)[C@@H](CN2C[C@H]3C[C@@H]2CO3)CN1] 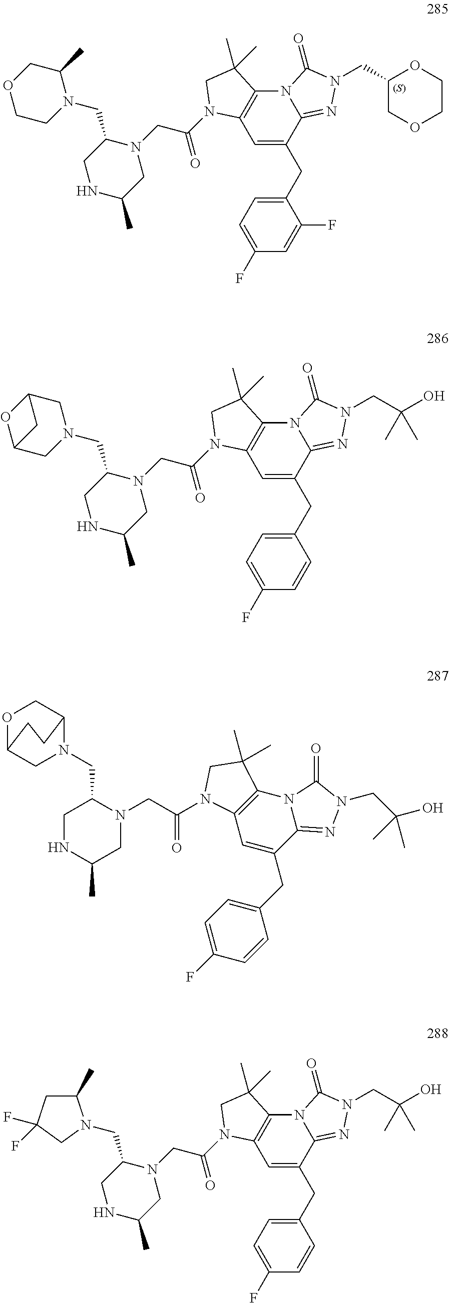 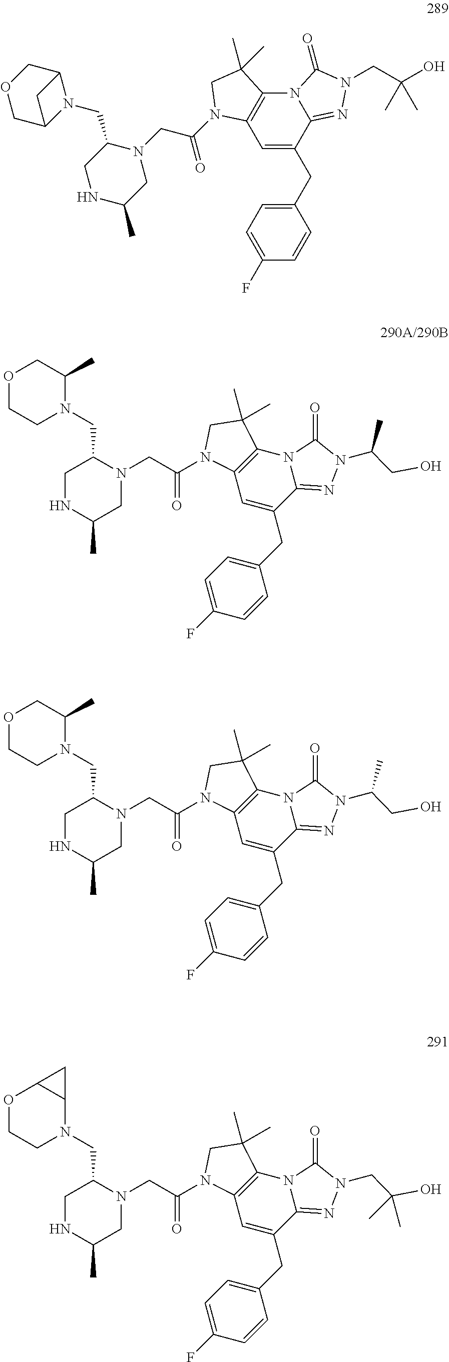 [SMILES: C[C@@H]1CN(CC(=O)N2CC(C)(C)c3c2cc(Cc2ccc(F)cc2)c2nn(CC(C)(C)O)c(=O)n32)[C@@H](CN2C3COCC2C3)CN1 ; C[C@@H]1CN(CC(=O)N2CC(C)(C)c3c2cc(Cc2ccc(F)cc2)c2nn([C@@H](C)CO)c(=O)n32)[C@@H](CN2CCOC[C@H]2C)CN1 ; C[C@@H]1CN(CC(=O)N2CC(C)(C)c3c2cc(Cc2ccc(F)cc2)c2nn([C@H](C)CO)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CC(C)(C)O)c(=O)n32)[C@@H](CN2CCOC3CC32)CN1] 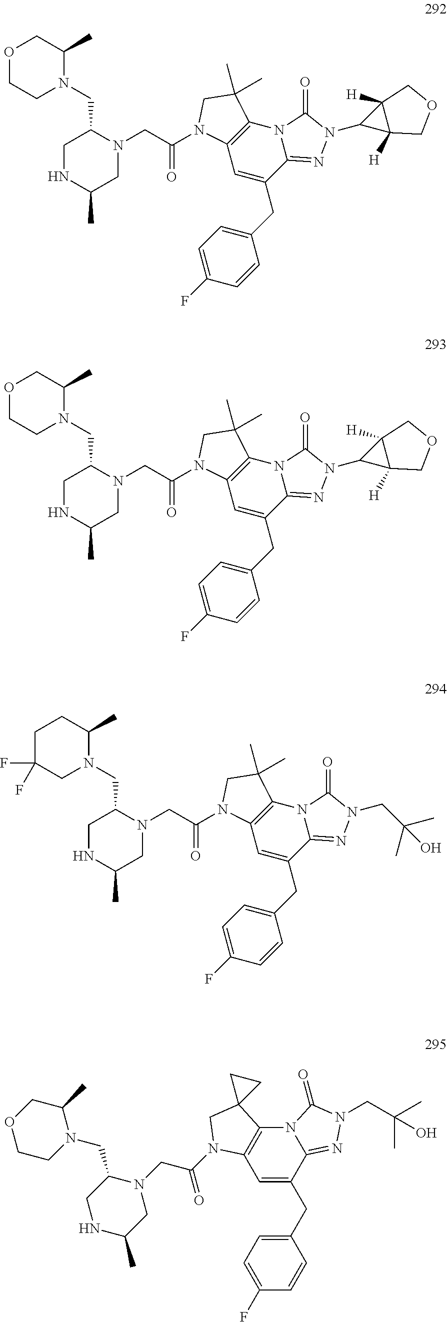 [SMILES: C[C@@H]1CN(CC(=O)N2CC(C)(C)c3c2cc(Cc2ccc(F)cc2)c2nn(C4[C@H]5COC[C@@H]45)c(=O)n32)[C@@H](CN2CCOC[C@H]2C)CN1 ; C[C@@H]1CN(CC(=O)N2CC(C)(C)c3c2cc(Cc2ccc(F)cc2)c2nn(C4[C@H]5COC[C@@H]45)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CC(C)(C)O)c(=O)n32)[C@@H](CN2CC(F)(F)CC[C@H]2C)CN1 ; C[C@@H]1CN(CC(=O)N2CC3(CC3)c3c2cc(Cc2ccc(F)cc2)c2nn(CC(C)(C)O)c(=O)n32)[C@@H](CN2CCOC[C@H]2C)CN1] 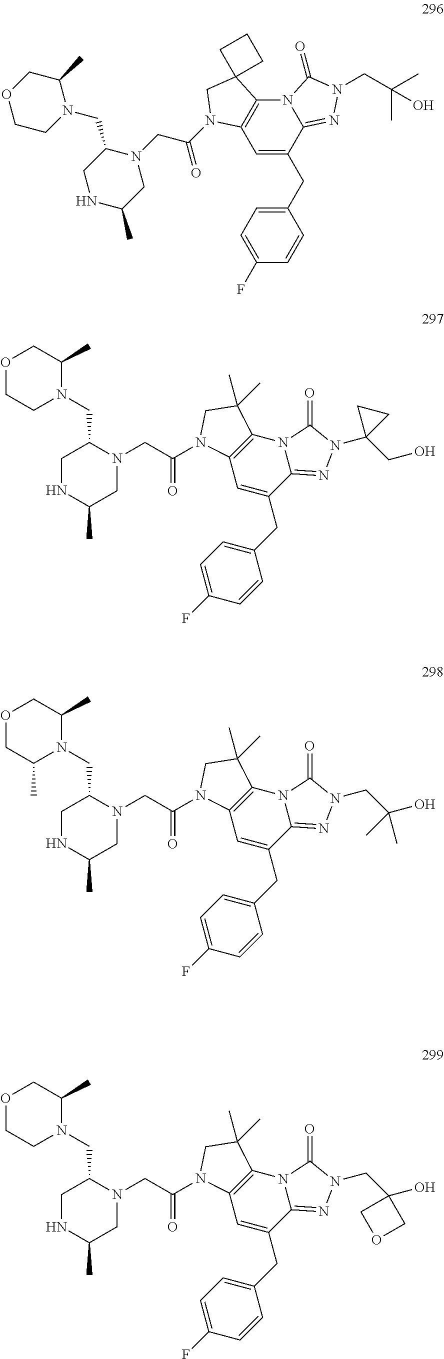 [SMILES: C[C@@H]1CN(CC(=O)N2CC3(CCC3)c3c2cc(Cc2ccc(F)cc2)c2nn(CC(C)(C)O)c(=O)n32)[C@@H](CN2CCOC[C@H]2C)CN1 ; C[C@@H]1CN(CC(=O)N2CC(C)(C)c3c2cc(Cc2ccc(F)cc2)c2nn(C4(CO)CC4)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CC(C)(C)O)c(=O)n32)[C@@H](CN2[C@H](C)COC[C@H]2C)CN1 ; C[C@@H]1CN(CC(=O)N2CC(C)(C)c3c2cc(Cc2ccc(F)cc2)c2nn(CC4(O)COC4)c(=O)n32)[C@@H](CN2CCOC[C@H]2C)CN1] 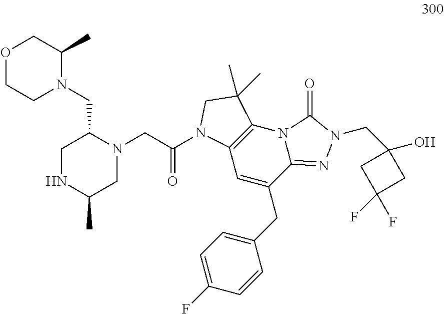 [SMILES: C[C@@H]1CN(CC(=O)N2CC(C)(C)c3c2cc(Cc2ccc(F)cc2)c2nn(CC4(O)CC(F)(F)C4)c(=O)n32)[C@@H](CN2CCOC[C@H]2C)CN1] 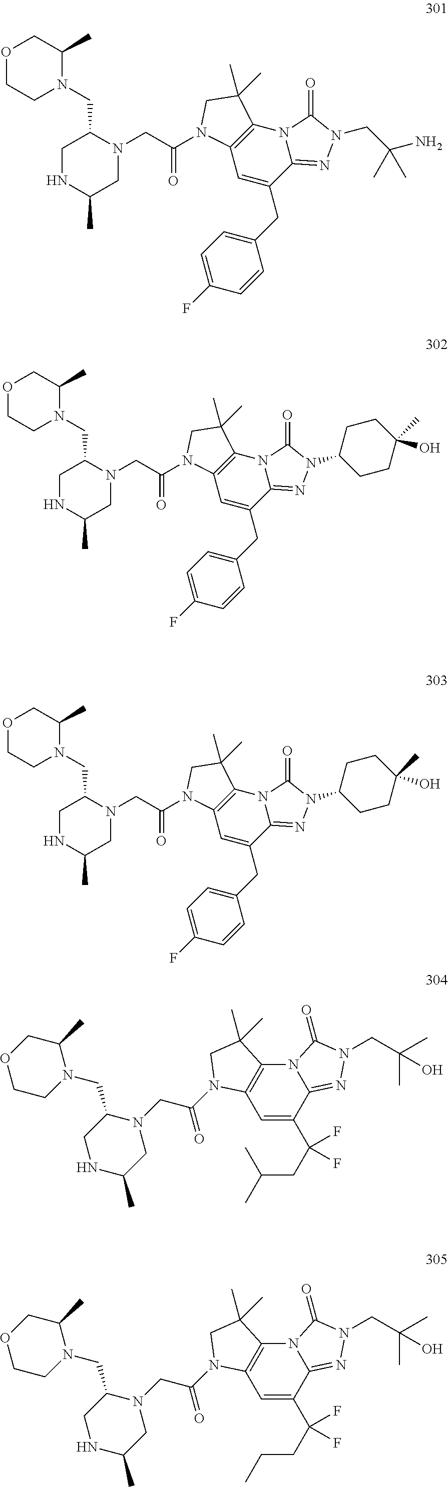 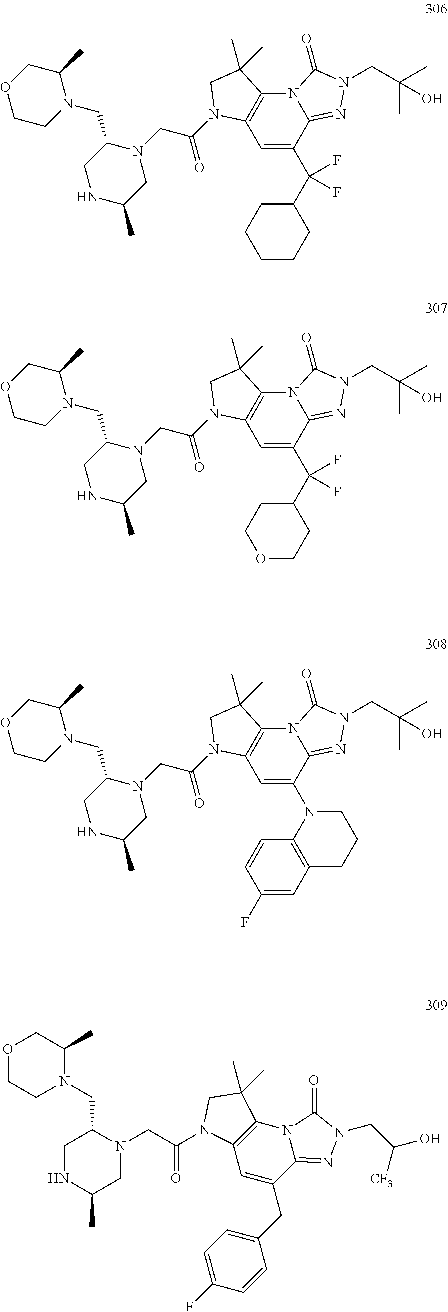 [SMILES: C[C@@H]1CN(CC(=O)N2CC(C)(C)c3c2cc(C(F)(F)C2CCCCC2)c2nn(CC(C)(C)O)c(=O)n32)[C@@H](CN2CCOC[C@H]2C)CN1 ; C[C@@H]1CN(CC(=O)N2CC(C)(C)c3c2cc(C(F)(F)C2CCOCC2)c2nn(CC(C)(C)O)c(=O)n32)[C@@H](CN2CCOC[C@H]2C)CN1 ; C[C@@H]1CN(CC(=O)N2CC(C)(C)c3c2cc(N2CCCc4cc(F)ccc42)c2nn(CC(C)(C)O)c(=O)n32)[C@@H](CN2CCOC[C@H]2C)CN1 ; C[C@@H]1CN(CC(=O)N2CC(C)(C)c3c2cc(Cc2ccc(F)cc2)c2nn(CC(O)C(F)(F)F)c(=O)n32)[C@@H](CN2CCOC[C@H]2C)CN1]
or a pharmaceutically acceptable salt or solvate thereof.

26. (canceled)

27. A pharmaceutical composition comprising the compound of claim 1, or a pharmaceutically acceptable salt or solvate thereof, and a pharmaceutically acceptable carrier.

28. A method for inhibiting IAP protein activity in a cell, comprising contacting the cell in which inhibition of IAP protein activity is desired with an effective amount of a compound of claim 1, or a pharmaceutically acceptable salt or solvate thereof.

29. A method of treating a disease or condition wherein inhibition of an IAP protein provides a benefit comprising administering a therapeutically effective amount of a compound of claim 1, or a pharmaceutically acceptable salt or solvate thereof; to an individual in need thereof.

30-37. (canceled)

38. A kit comprising a compound of claim 1, and instructions for administering the compound, or a pharmaceutically acceptable salt or solvate thereof, to a subject for which the inhibition of an IAP protein provides a benefit.

39. (canceled)
